(19) United States
(12) Patent Application Publication
(10) Pub. No.: US 2025/0353841 A1
(43) Pub. Date: Nov. 20, 2025

(54) HETEROCYCLIC COMPOUNDS FOR TREATING HUNTINGTON'S DISEASE

(71) Applicants: Sarah Elizabeth HUFF, Cambridge, MA (US); Darsheed Nasser MUSTAFA, Cambridge, MA (US); Adam TOSZEWSKI, Cambridge, MA (US); Dibyendu MONDAL, Cambridge, MA (US); BIOGEN MA INC., Cambridge, MA (US)

(72) Inventors: Magnus Pfaffenbach, Chelsea, MA (US); James Harvey Harvey, Arlington, MA (US); Daniel R. Smith, Dorchester, MA (US); Philippe Bolduc, Weymouth, MA (US); Nupur Bansal, Medfield, MA (US); Chaofan Xu, Newton, MA (US); Emily Anne Peterson, Belmont, MA (US); Sarah Elizabeth Huff, Cambridge, MA (US); Darsheed Nasser Mustafa, Watertown, MA (US); Adam Thomas Antoszewski, Cambridge, MA (US); Dibyendu Mondal, Somerville, MA (US)

(21) Appl. No.: 18/867,733

(22) PCT Filed: May 19, 2023
(86) PCT No.: PCT/US2023/022813

§ 371 Date: Nov. 20, 2024

Related U.S. Application Data
(60) Provisional application No. 63/344,494, filed on May 20, 2022.

(51) Int. Cl. C07D 471/04; A61K 31/437; A61K 31/439; A61K 31/454; A61K 31/4545; A61K 31/4725; A61K 31/4985; A61K 31/502; A61K 31/5025; A61K 31/506; A61K 31/519; A61K 31/53; A61K 31/5377; A61K 31/5383; A61K 31/55; C07B 59/00; C07D 401/14; C07D 413/14; C07D 487/04; C07D 519/00

(52) U.S. Cl. CPC C07D 471/04; A61K 31/437; A61K 31/439; A61K 31/454; A61K 31/4545; A61K 31/4725; A61K 31/4985; A61K 31/502; A61K 31/5025; A61K 31/506; A61K 31/519; A61K 31/53; A61K 31/5377; A61K 31/5383; A61K 31/55; C07B 59/002; C07D 401/14; C07D 413/14; C07D 487/04; C07D 519/00

(57) ABSTRACT

The present disclosure provides a compound of Formula (I) or a pharmaceutically acceptable salt thereof and its use in, e.g. treating a condition, disease, or disorder in which lowering mutant huntingtin protein (“mHTT”) in a subject is of therapeutic benefit, specifically in treating Huntington disease (“HD”). This disclosure also features a composition containing the same as well as methods of using and making the same.

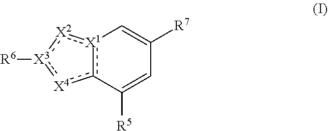

### RELATED APPLICATION

This application claims the benefit of the filing date, under 35 U.S.C. § 119(e), of U.S. Provisional Application No. 63/344,494, filed on May 20, 2022, the entire contents of which are incorporated here by reference.

### BACKGROUND

Huntington's disease (HD) is an autosomal dominant progressive neurodegenerative disorder, which has a prevalence of between three and seven individuals per 100,000 worldwide. HD is caused by cytosine-adenine-guanine (CAG) repeat expansions in the huntingtin (HTT) gene resulting in the production of a ubiquitously expressed pathogenic mutant HTT (mHTT) protein. Mutant huntingtin contains an abnormally long polyglutamine (polyQ) sequence that corresponds to the CAG genetic expansion; the protein exhibits toxic properties that cause dysfunction and death of neurons. The disease is characterized by motor, cognitive, psychiatric and functional capacity decline.

Some research progresses are being made in identifying HTT protein-lowering therapies using multiple tools, including ribonucleic acid (RNA) interference using short interfering RNAs, short-hairpin RNAs, or microRNAs and antisense oligonucleotides (“ASO”) causing translational repression or messenger RNA (mRNA) degradation. However, these therapies require either surgical delivery of a viral vector for chronic HTT transcript lowering by RNAi, or repeated infusions into the cerebral spinal fluid (“CSF”) by lumbar puncture for ASOs in the clinic.

More recently, a small molecule compound platform, which modulates RNA expression, i.e. splicing correction, is under development. NVS-SM1 (LMI070), now called branaplam, is a pyridazine derivative. It is reported that branaplam lowers mHTT protein levels in HD patient cells, in an HD mouse model and in blood samples from Spinal Muscular Atrophy (SMA) Type I patients dosed orally for SMA (NCT02268552). See Keller, C. etc., An Orally Available, Brain Penetrant, Small Molecule Lowers Huntingtin Levels by Enhancing Pseudoexon Inclusion, Nature Communications, (2022) 13:1150.

However, there are no approved disease-modifying treatments for HD till now, leaving a high unmet need for medications that can be used for treating or ameliorating HD. Accordingly, there is a need to find disease-modifying therapies for HD (i.e. therapeutic options that can slow disease progression).

### SUMMARY

Described herein are compounds or pharmaceutically acceptable salts thereof, which can be useful in treating HD in a subject.

In one aspect, the present disclosure provides a compound of Formula (I) or a pharmaceutically acceptable salt thereof:

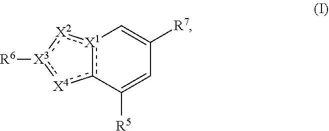

wherein X¹, X², X³, X⁴, R⁵, R⁶, and R⁷ are as defined herein.

Also provided are pharmaceutical compositions comprising a compound of Formula (I) or a pharmaceutically acceptable salt thereof and a pharmaceutically acceptable carrier or excipient.

The present disclosure further provides methods of lowering mHTT in a subject, comprising administering to the subject a compound of Formula (I) or a pharmaceutically acceptable salt thereof.

The present disclosure also provides methods of treating a disease or condition modulated at least in part by mHTT in a subject, comprising administering to the subject a therapeutically effective amount of a compound of Formula (I) or a pharmaceutically acceptable salt thereof.

The present disclosure further provides a method of treating Huntington disease (“HD”) in a subject in need thereof, comprising administering to the subject an effective amount of (1) a compound of Formula (I) or a pharmaceutically acceptable salt thereof, or (2) a pharmaceutically acceptable composition comprising a compound of Formula (I) or a pharmaceutically acceptable salt thereof, and a pharmaceutically acceptable carrier.

In certain embodiments of the methods of the present disclosure, HD can be treated by lowering mHTT level in a subject.

The present disclosure also provides a use of a compound of Formula (I), a pharmaceutically acceptable salt, or a pharmaceutical composition comprising the same in any of the methods described herein. In one embodiment, provided is a compound of Formula (I) or a pharmaceutically acceptable salt thereof or a pharmaceutical composition comprising the same for use in any of the methods described herein. In another embodiment, provided is use of a compound of Formula (I) or a pharmaceutically acceptable salt thereof or a pharmaceutical composition comprising the same for the manufacture of a medicament for any of the methods described herein.

### DETAILED DESCRIPTION

### 1. Compounds

In a first aspect, the present disclosure provides a compound of Formula (I):

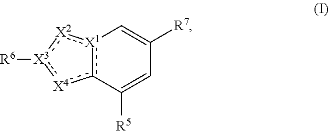

or a pharmaceutically acceptable salt thereof, wherein:
[symbol] is a single bond or double bond, provided the ring containing X₁, X₂, X₃, and X₄ is a bicyclic heteroaryl ring comprising at least one N atom;
X¹ is C or N;
X² is O, N or CR²;
X³ is N or C;
X⁴ is N, O, NR⁴ or CR⁴; provided when X¹ is C, at least two of X², X³ and X⁴ are O, N or NR⁴;
R² and R⁴, when present, are each independently selected from a group consisting of H, halo, and C₁₋₆alkyl;
R⁵ is H, halo, hydroxyl, C₁₋₆alkyl, C₁₋₆haloalkyl, C₁₋₆alkoxyl, or C₁₋₆haloalkoxyl;
R⁶ is A, —N(R^{6a})-A, —C(═O)A, —N(R^{6a})C(═O)-A, or —C(═O)N(R^{6a})-A and R⁷ is B; additionally R⁶ is B and R⁷ is A when X¹ is N; wherein
   R^{6a} is H or C₁₋₃alkyl;
   A is —C₁₋₆alkylene-NR⁹R¹⁰, 4 to 10 membered carbocyclyl, —C₁₋₆alkylene-(4 to 10 membered carbocyclyl), Het or —C₁₋₆alkylene-Het; wherein:
      R⁹ is H or C₁₋₆alkyl;
      R¹⁰ is H, C₁₋₆alkyl, C₃₋₆cycloalkyl, —C₁₋₆alkylene-C₃₋₆cycloalkyl, or —C₁₋₆alkylene-Het¹, wherein Het¹ is a 4-6 membered saturated heterocyclyl;
      Het is a 4 to 12 membered saturated heterocyclyl optionally substituted with —NR⁹R¹⁰ or —C₁₋₆alkylene-NR⁹R¹⁰ and optionally further substituted with 1 to 4 R¹¹;
      said 4 to 10 membered carbocyclyl represented by A is optionally substituted by —NR⁹R¹⁰ or —C₁₋₆alkylene-NR⁹R¹⁰ and is further optionally substituted with 1 to 2 R¹¹; wherein:
         R¹¹, for each occurrence, is independently selected from halo, —C(═O)R¹², C₁₋₆alkyl, C₁₋₆haloalkyl, C₁₋₆alkoxyC₁₋₆alkyl, C₃₋₆cycloalkyl, —C₁₋₆alkylene-C₃₋₆cycloalkyl, Het², and —C₁₋₆alkylene-Het², wherein Het² is a 4 to 6 membered saturated heterocyclyl or 5 to 10 member heteroaryl, wherein said Het² or C₃₋₆cycloalkyl is optionally substituted by one or more substituents independently selected from halo, C₁₋₆alkoxy and C₁₋₆alkyl; wherein R¹² is H, D, halo, C₁₋₃alkyl, C₁₋₆alkoxyl, or C₃₋₆cycloalkyl;
   B is 6 to 10 membered aryl, 4 to 10 membered heterocyclyl, or 5 to 10 member heteroaryl, wherein said 6 to 10 membered aryl, 4 to 10 membered heterocyclyl, and 5 to 10 member heteroaryl represented by B are optionally substituted by one or more R⁸; wherein
      R⁸ is halo, —CN, —OH, C₁₋₆alkyl, C₃₋₆cycloalkyl, 5 or 6 membered heteroaryl, C₁₋₆haloalkyl, or C₁₋₆alkoxy, or two R⁸ together with the intervening atoms together form a 4 to 7 membered heterocyclyl optionally substituted with one or more R^{8b}; wherein said 5 or 6 membered heteroaryl represented by R⁸ is optionally substituted by one or more R^{8a}; wherein R^{8a} is C₁₋₃alkyl; and R^{8b} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur.

In a first embodiment, the present disclosure provides a compound of Formula (I):

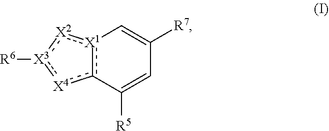

or a pharmaceutically acceptable salt thereof, wherein:
[symbol] is a single bond or double bond, provided the ring containing X₁, X₂, X₃, and X₄ is a bicyclic heteroaryl ring comprising at least one N atom;
X¹ is C or N;
X² is O, N or CR²;
X³ is N or C;
X⁴ is N, NR⁴ or CR⁴; provided when X¹ is C, at least two of X², X³ and X⁴ are O, N or NR⁴;
R² and R⁴, when present, are each independently selected from a group consisting of H, halo, and C₁₋₆alkyl;
R⁵ is halo;
R⁶ is A, —N(R^{6a})C(═O)-A, or —C(═O)N(R^{6a})-A and R⁷ is B; or
R⁶ is B and R⁷ is A when X¹ is N; wherein
   R^{6a} is H or C₁₋₃alkyl;
   A is —C₁₋₆alkylene-NR⁹R¹⁰, 4 to 10 membered saturated carbocyclyl, Het or —C₁₋₆alkylene-Het; wherein
      R⁹ is H or C₁₋₆alkyl;
      R¹⁰ is H, C₁₋₆alkyl or —C₁₋₆alkylene-Het¹, wherein Het¹ is a 4-6 membered saturated heterocyclyl;
      Het is a 4 to 10 membered saturated heterocyclyl, provided when said 4 to 10-membered saturated heterocyclyl represented by Het does not comprise a ring N atom, it is then substituted with —NR⁹R¹⁰ and optionally further substituted with 1 to 2 R¹¹, and when the 4 to 10-membered saturated heterocyclyl represented by Het comprises one or more ring N atoms, it is optionally substituted with 1 to 3 R¹¹;
      said 4 to 10 membered saturated carbocyclyl represented by A is substituted by —NR⁹R¹⁰ and is further optionally substituted with 1 to 2 R¹¹; wherein
         R¹¹, for each occurrence, is independently selected from halo, —C(═O)R¹², C₁₋₆alkyl, C₁₋₆haloalkyl, C₁₋₆alkoxyC₁₋₆alkyl, and C₃₋₆cycloalkyl; wherein said C₃₋₆cycloalkyl represented by R¹¹ is optionally substituted by one or more substituents independently selected from halo and C₁₋₆alkyl; wherein R¹² is H, C₁₋₃alkyl, or C₃₋₆cycloalkyl;
   B is 6 to 10 membered aryl, 4 to 10 membered heterocyclyl, or 5 to 10 membered heteroaryl, wherein said 6 to 10 membered aryl, 4 to 10 membered heterocyclyl, and 5 to 10 membered heteroaryl represented by B are optionally substituted by one or more R⁸; wherein
      R⁸ is halo, —CN, —OH, C₁₋₆alkyl, C₁₋₆haloalkyl, or C₁₋₆alkoxy, or two R⁸ together with the intervening atoms together form a 5 to 7 membered heterocyclyl optionally substituted with one or more R^{8b}; wherein said 5 or 6 membered heteroaryl represented by R⁸ is optionally substituted by one or more R^{8a}; wherein R^{8a} is C₁₋₃alkyl; and R^{8b} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur.

In a second embodiment, the present disclosure provides a compound according to the first aspect or the first embodiment or a pharmaceutically acceptable salt thereof, wherein the compound is represented by Formula (II), (III), (IV), (V), (VI), (VII), (VIII), or (IX):

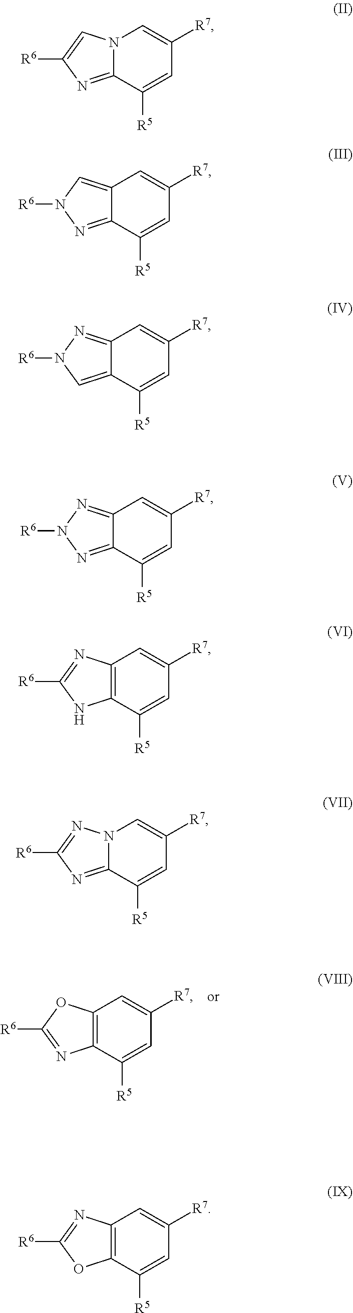

The definitions of the variables are provided in the first aspect or the first embodiment.

In an alternative second embodiment, the present disclosure provides a compound according to the first aspect or the first embodiment or a pharmaceutically acceptable salt thereof, wherein the compound is represented by Formula (II), (III), (IV), (V), (VI), (VII), or (VIII):

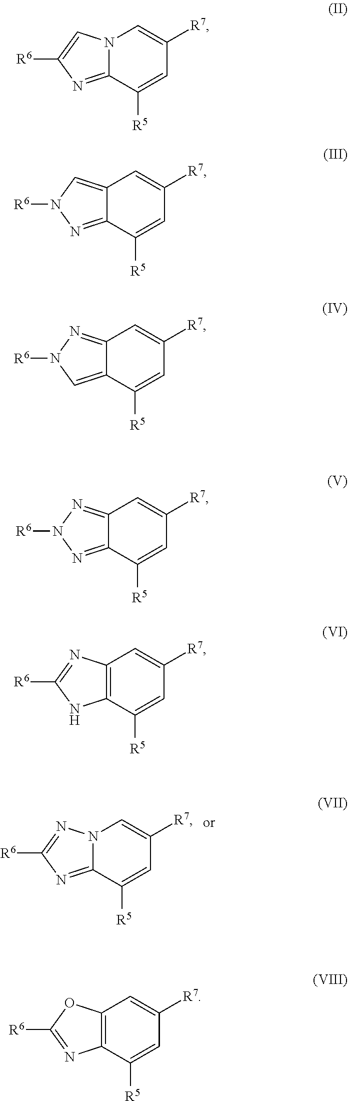

The definitions of the variables are provided in the first aspect or the first embodiment.

In a third embodiment, the present disclosure provides a compound according to the first aspect or the first or the second embodiment or a pharmaceutically acceptable salt thereof, wherein the compound is represented by Formula (II):

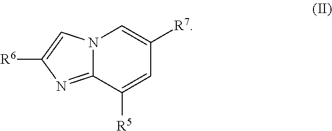

The definitions of the variables are provided in the first aspect, or in the first or second embodiment.

In a fourth embodiment, the present disclosure provides a compound according to the first aspect or any one of the first through third embodiments or a pharmaceutically acceptable salt thereof, wherein R⁵ is H, halo, C₁₋₃alkyl, C₁₋₃haloalkoxyl or C₁₋₃alkoxyl. The definitions of the remaining variables are provided in the first aspect or in any one of the first through third embodiments or any alternative embodiments described therein.

In an alternative fourth embodiment, the present disclosure provides a compound according to the first aspect or any one of the first through third embodiments or a pharmaceutically acceptable salt thereof, wherein R⁵ is F or Cl. The definitions of the remaining variables are provided in the first aspect or any one of the first through third embodiments or any alternative embodiments described therein.

In a fifth embodiment, the present disclosure provides a compound according to the first aspect or any one of the first through the third embodiments or a pharmaceutically acceptable salt thereof, wherein R⁵ is H, F, Cl, —CH₃, —OCHF₂, —OCH₃ or —OCF₃. The definitions of the remaining variables are provided in the first aspect or any one of the first through third embodiments or any alternative embodiments described therein.

In an alternative fifth embodiment, the present disclosure provides a compound according to the first aspect or any one of the first through the third embodiments or a pharmaceutically acceptable salt thereof, wherein R⁵ is F. The definitions of the remaining variables are provided in the first aspect or any one of the first through third embodiments or any alternative embodiments described therein.

In a sixth embodiment, the present disclosure provides a compound according to the first aspect or any one of the first through fifth embodiments or a pharmaceutically acceptable salt thereof, wherein R⁶ is A, —N(R^{6a})-A, —N(R^{6a})C(═O)-A, or —C(═O)N(R^{6a})-A; R⁷ is B and R^{6a} is H or —CH₃. The definitions of the remaining variables are provided in the first aspect or any one of the first through fifth embodiments or any alternative embodiments described therein.

In an alternative sixth embodiment, the present disclosure provides a compound according to the first aspect or any one of the first through fifth embodiments or a pharmaceutically acceptable salt thereof, wherein R⁶ is A, —NHC(═O)-A, or —C(═O)NH-A and R⁷ is B. The definitions of the remaining variables are provided in the first aspect or any one of the first through fifth embodiments or any alternative embodiments described therein.

In a seventh embodiment, the present disclosure provides a compound according to the first aspect or any one of the first through fifth embodiments or a pharmaceutically acceptable salt thereof, wherein R⁶ is B and R⁷ is A when X¹ is N. The definitions of the remaining variables are provided in the first aspect or any one of the first through fifth embodiments or any alternative embodiments described therein.

In an eighth embodiment, the present disclosure provides a compound according to the seventh embodiment or a pharmaceutically acceptable salt thereof, wherein
X¹ is N;
X² is CR²;
X³ is C;
X is N;
R⁶ is B; and
R⁷ is A.
   The definitions of the remaining variables are provided in the seventh embodiment.

In a ninth embodiment, the present disclosure provides a compound according to the first aspect or any one of the first through the sixth embodiments or a pharmaceutically acceptable salt thereof, wherein R⁶ is A when R⁷ is B. The definitions of the remaining variables are provided in the first aspect or any one of the first through the sixth embodiments or any alternative embodiments described therein.

In a tenth embodiment, the present disclosure provides a compound according to the first aspect or any one of the first through the sixth embodiments or a pharmaceutically acceptable salt thereof, wherein R⁶ is —N(R^{6a})-A, —N(R^{6a})C(═O)-A or —C(═O)N(R^{6a})-A and R⁷ is B when X¹ is N; and wherein R^{6a} is H or —CH₃. The definitions of the remaining variables are provided in the first aspect or any one of the first through the sixth embodiments or any alternative embodiments described therein.

In an alternative tenth embodiment, the present disclosure provides a compound according to the first aspect or any one of the first through the sixth embodiments or a pharmaceutically acceptable salt thereof, wherein R⁶ is —NHC(═O)-A or —C(═O)NH-A and R⁷ is B when X¹ is N. The definitions of the remaining variables are provided in the first aspect or any one of the first through the sixth embodiments or any alternative embodiments described therein.

In an eleventh embodiment, the present disclosure provides a compound according to the tenth embodiment or a pharmaceutically acceptable salt thereof, wherein
X¹ is N;
X² is CR²;
X³ is C;
X is N;
R⁶ is —N(R^{6a})-A, —N(R^{6a})C(═O)-A or —C(═O)N(R^{6a})-A;
R^{6a} is H or —CH₃; and
R⁷ is B.

The definitions of the remaining variables are provided in the tenth embodiment or any alternative embodiments described therein.

In an alternative eleventh embodiment, the present disclosure provides a compound according to the tenth embodiment or a pharmaceutically acceptable salt thereof, wherein
X¹ is N;
X² is CR²;
X³ is C;
X⁴ is N;
R⁶ is —NHC(═O)-A or —C(═O)NH-A; and
R⁷ is B.
   The definitions of the remaining variables are provided in the tenth embodiment or any alternative embodiments described therein.

In a twelfth embodiment, the present disclosure provides a compound according to the first aspect or any one of the first through the eleventh embodiments or a pharmaceutically acceptable salt thereof, wherein:
A is —C₁₋₆alkylene-C₃₋₆cycloalkyl, —C₁₋₆alkylene-NR⁹R¹⁰ or —C₁₋₆alkylene-Het; wherein
   said Het in —C₁₋₆alkylene-Het represented by A is a 4 to 6 membered monocyclic saturated heterocyclyl comprising a ring N atom; and
   R⁹ and R¹⁰ are each independently H or C₁₋₄alkyl.
      The definitions of the remaining variables are provided in the first aspect, or any one of the first through the eleventh embodiments or any alternative embodiments described therein.

In a thirteenth embodiment, the present disclosure provides a compound according to the twelfth embodiment or a pharmaceutically acceptable salt thereof, wherein Het in —C₁₋₆alkylene-Het represented by A is azetidinyl, pyrrolidinyl, piperidinyl, morpholinyl or piperazinyl. The definitions of the remaining variables are provided in the twelfth embodiment.

In a fourteenth embodiment, the present disclosure provides a compound according to the twelfth embodiment or a pharmaceutically acceptable salt thereof, wherein A is selected from a group consisting of

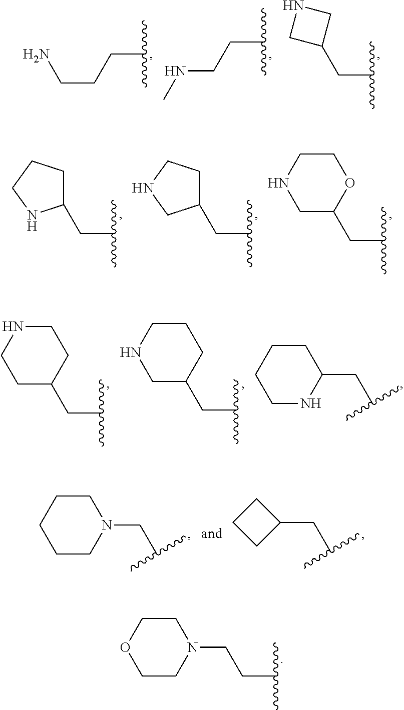

The definitions of the remaining variables are provided in the twelfth embodiment.

In an alternative fourteenth embodiment, the present disclosure provides a compound according to the twelfth embodiment or a pharmaceutically acceptable salt thereof, wherein A is selected from a group consisting of

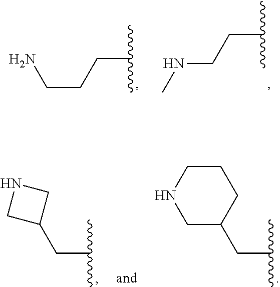

The definitions of the remaining variables are provided in the twelfth embodiment.

In a fifteenth embodiment, the present disclosure provides a compound according to the first aspect, or any one of the first through the eleventh embodiments or a pharmaceutically acceptable salt thereof, wherein:
A is 5 to 6 membered monocyclic carbocyclyl, 5 to 8 membered bicyclic saturated bridged carbocyclyl, or Het;
Het represented by A is a 4 to 7 membered monocyclic saturated heterocyclyl, 6 to 8 membered bicyclic saturated bridged heterocyclyl, or 7 to 12 membered bicyclic saturated spiral or fused heterocyclyl; provided when the Het represented by A does not comprise a ring N atom, it is then substituted with —NR⁹R¹⁰ or —C₁₋₆alkylene-NR⁹R¹⁰ and optionally further substituted with 1 to 2 R¹¹, and when the Het represented by A comprises one or more ring N atoms, it is optionally substituted with 1 to 2 R¹¹; and
said 5 to 8 membered bicyclic saturated bridged carbocyclyl represented by A is substituted by —NR⁹R¹⁰, 4 to 6 membered monocyclic saturated heterocyclyl, or —C₁₋₆alkylene-NR⁹R¹⁰ and is optionally further substituted with 1 to 2 R¹¹; and
R⁹ and R¹⁰ are each independently H or C₁₋₄alkyl.

The definitions of the remaining variables are provided in the first aspect or any one of the first through the eleventh embodiments or any alternative embodiments described therein.

In an alternative fifteenth embodiment, the present disclosure provides a compound according to the first aspect or any one of the first through the eleventh embodiments or a pharmaceutically acceptable salt thereof, wherein:
A is 5 to 6 membered monocyclic saturated carbocyclyl, 5 to 8 membered bicyclic saturated bridged carbocyclyl, or Het;
Het represented by A is a 4 to 6 membered monocyclic saturated heterocyclyl, 6 to 8 membered bicyclic saturated bridged heterocyclyl, or 7 to 10 membered bicyclic saturated spiral heterocyclyl; provided when the Het represented by A does not comprise a ring N atom, it is then substituted with —NR⁹R¹⁰ and optionally further substituted with 1 to 2 R¹¹, and when the Het represented by A comprises one or more ring N atoms, it is optionally substituted with 1 to 2 R¹¹; and
said 5 to 8 membered bicyclic saturated bridged carbocyclyl represented by A is substituted by —NR⁹R¹⁰ and is optionally further substituted with 1 to 2 R¹¹;
R⁹ and R¹⁰ are each independently H or C₁₋₄alkyl.
   The definitions of the remaining variables are provided in the first aspect or any one of the first through the eleventh embodiments or any alternative embodiments described therein.

In a sixteenth embodiment, the present disclosure provides a compound according to the fifteenth embodiment or a pharmaceutically acceptable salt thereof, wherein A is azetidinyl, pyrrolidinyl, piperidinyl, morpholinyl, piperazinyl, 2-azabicyclo[2.1.1]hexyl, 3-azabicyclo[3.1.1]heptanyl, 2-azabicyclo[3.1.1]heptanyl, 2-azabicyclo[2.2.1]heptanyl, 1-azaspiro[3.3]heptanyl, 2-azaspiro[4.5]decanyl, 4-azaspiro[2.5]octanyl, 8-azaspiro[4.5]decanyl, 8-azabicyclo[3.2.1]octanyl, 3-azabicyclo[3.2.1]octanyl, 9-diazaspiro[5.5]undecanyl, 2-azabicyclo[4.1.0]heptanyl, 3-azabicyclo[4.1.0]heptanyl, 5-azaspiro[2.4]heptanyl, 5-azaspiro[2.3]hexanyl, 4-azaspiro[2.4]heptanyl, 6-azaspiro[3.4]octanyl, 2-azaspiro[4.4]nonanyl, 2-azaspiro[3.5]nonanyl, 2-azaspiro[3.4]octanyl, 1-oxa-9-azaspiro[5.5]undecanyl, 3-azabicyclo[3.1.0]hexanyl, diazaspiro[4.5]decane, 7-diazaspiro[3.5]nonanyl, diazaspiro[4.5]decanyl, 7-diazaspiro[4.4]nonanyl, 1-azabicyclo[3.2.1]octanyl, diazaspiro[5.5]undecanyl, azepanyl, 7-azaspiro[3.5]nonanyl, 7-azaspiro[3.5]nonanyl, 5-oxa-2-azaspiro[3.4]octanyl, diazabicyclo[3.2.0]heptanyl, 3-azabicyclo[3.2.0]heptanyl, octahydro-cyclopenta[c]pyrrolyl, hexahydro-1H-pyrrolo[3,4-c]pyrrolyl, octahydro-indolizinyl, 8-diazabicyclo[4.2.0]octanyl, octahydro-isoindolyl, or 1,8-diazaspiro[4.5]decane, each of which is optionally substituted with one or two R¹¹. The definitions of the remaining variables are provided in the fifteenth embodiment or any alternative embodiments described therein.

In an alternative sixteenth embodiment, the present disclosure provides a compound according to the fifteenth embodiment or a pharmaceutically acceptable salt thereof, wherein A is azetidinyl, pyrrolidinyl, piperidinyl, morpholinyl, piperazinyl, 2-azabicyclo[2.1.1]hexyl, 3-azabicyclo[3.1.1]heptanyl, 2-azabicyclo[3.1.1]heptanyl, 2-azabicyclo[2.2.1]heptanyl, 1-azaspiro[3.3]heptanyl, 2-azaspiro[4.5]decanyl, 4-azaspiro[2.5]octanyl, 8-azaspiro[4.5]decanyl, 8-azabicyclo[3.2.1]octanyl, or 7-azaspiro[3.5]nonanyl, each of which is optionally substituted with one or two R¹¹. The definitions of the remaining variables are provided in the fifteenth embodiment or any alternative embodiments described therein.

In a seventeenth embodiment, the present disclosure provides a compound according to the fifteenth embodiment or the sixteenth embodiment or a pharmaceutically acceptable salt thereof, wherein A is selected from a group consisting of:

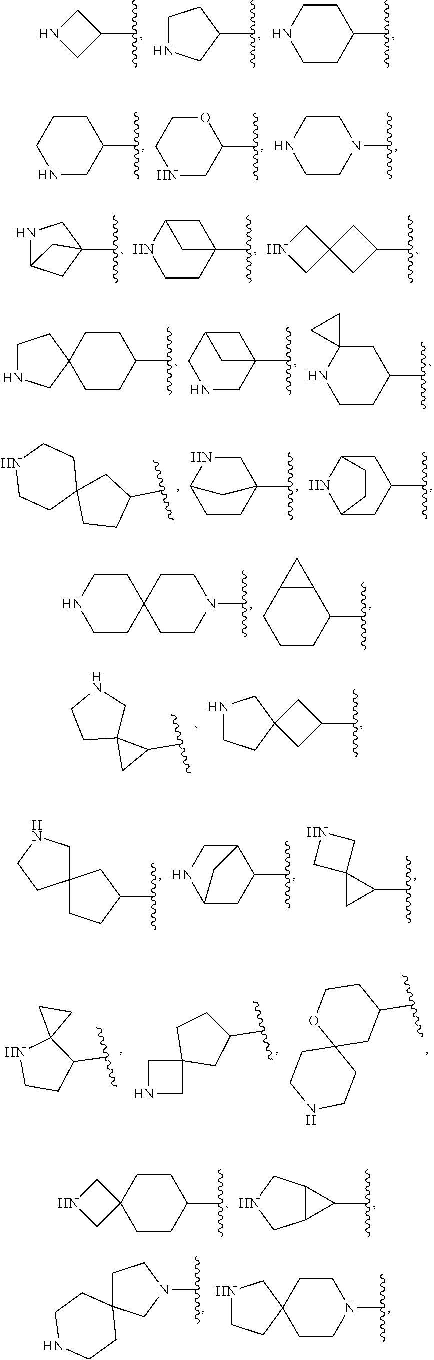

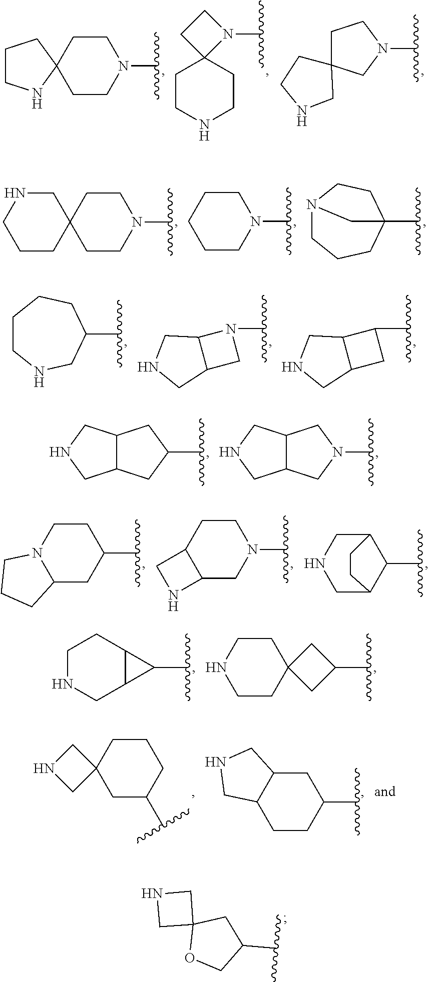

each of which is optionally substituted with one or two R¹¹. The definitions of the remaining variables are provided in the fifteenth or sixteenth embodiment or any alternative embodiments described therein.

In an alternative seventeenth embodiment, the present disclosure provides a compound according to the fifteenth embodiment or the sixteenth embodiment or a pharmaceutically acceptable salt thereof, wherein A is selected from a group consisting of:

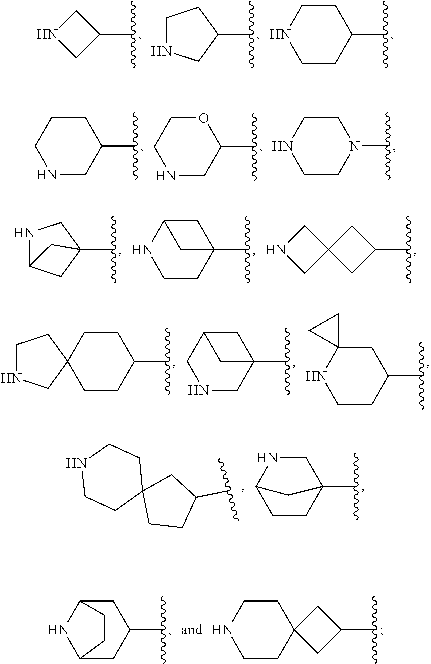

each of which is optionally substituted with one or two R¹¹. The definitions of the remaining variables are provided in the fifteenth or sixteenth embodiment or any alternative embodiments described therein.

In an eighteenth embodiment, the present disclosure provides a compound according to the fifteenth embodiment or a pharmaceutically acceptable salt thereof, wherein A is cyclobutyl, cyclopentyl, cyclopenetenyl, cyclohexyl, tetrahydro-2H-pyranyl, 3-oxetanyl, bicycle[1.1.1]pentyl, bicycle[2.1.1]hexyl, bicyclo[3.2.0]heptanyl, —CH₂-cyclobutyl, bicyclo[2.2.2]octanyl, spiro[5.3]nonanyl, or 2-oxobicyclo[2.1.1]hexyl, each of which is substituted with —NR⁹R¹⁰ and optionally further substituted with 1 to 2 R¹¹. The definitions of the remaining variables are provided in the fifteenth embodiment or any alternative embodiments described therein.

In an alternative eighteenth embodiment, the present disclosure provides a compound according to the fifteenth embodiment or a pharmaceutically acceptable salt thereof, wherein A is cyclopentyl, bicycle[1.1.1]pentyl, bicycle[2.1.1]hexyl, bicyclo[2.2.2]octanyl, or 2-oxobicyclo[2.1.1]hexyl, each of which is substituted with —NR⁹R¹⁰ and optionally further substituted with 1 to 2 R¹¹. The definitions of the remaining variables are provided in the fifteenth embodiment or any alternative embodiments described therein.

In a nineteenth embodiment, the present disclosure provides a compound according to the fifteenth embodiment or the eighteenth embodiment or a pharmaceutically acceptable salt thereof, wherein A is selected from a group consisting of

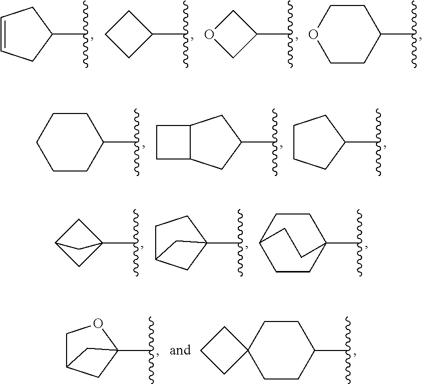

each of which is substituted with —NR⁹R¹⁰ and optionally further substituted with 1 to 2 R¹¹. The definitions of the remaining variables are provided in the fifteenth embodiment or any alternative embodiments described therein.

In an alternative nineteenth embodiment, the present disclosure provides a compound according to the fifteenth embodiment or the eighteenth embodiment or a pharmaceutically acceptable salt thereof, wherein A is selected from a group consisting of

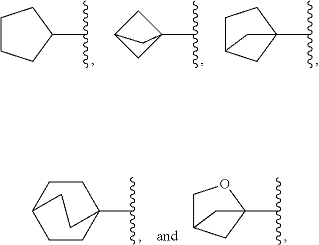

each of which is substituted with —NR⁹R¹⁰ and optionally further substituted with 1 to 2 R¹¹. The definitions of the remaining variables are provided in the fifteenth embodiment or any alternative embodiments described therein.

In a twentieth embodiment, the present disclosure provides a compound according to the first aspect, or any one of the first through the nineteenth embodiments or a pharmaceutically acceptable salt thereof, wherein R¹¹, for each occurrence, is independently selected from halo, —C(═O)R¹², C₁₋₆alkyl, C₁₋₄alkoxyC₁₋₄alkyl, C₃₋₆cycloalkyl, —C₁₋₆alkylene-C₃₋₆cycloalkyl, Het², and —C₁₋₆alkylene-Het², wherein Het² is a 4-6 membered saturated heterocyclyl or 5 to 6 membered heteroaryl; wherein said C₃₋₆cycloalkyl or Het² represented by R¹¹ is optionally substituted by one to four substituents independently selected from halo, C₁₋₄alkoxy and C₁₋₄alkyl; and R¹² is H, D, halo, C₁₋₄alkoxyl, C₁₋₂alkyl, C₃₋₄cycloalkyl. The definitions of the remaining variables are provided in the first aspect or any one of the first through the nineteenth embodiments or any alternative embodiments described therein.

In an alternative twentieth embodiment, the present disclosure provides a compound according to the first aspect or any one of the first through the nineteenth embodiments or a pharmaceutically acceptable salt thereof, wherein R¹¹, for each occurrence, is independently selected from halo, —C(═O)R¹², C₁₋₄alkyl, C₁₋₄alkoxyC₁₋₄alkyl, C₃₋₆cycloalkyl; wherein said C₃₋₆cycloalkyl represented by R¹¹ is optionally substituted by one to three substitutents independently selected from F, Cl, and C₁₋₄alkyl; and R¹² is H, C₁₋₂alkyl, C₃₋₄cycloalkyl. The definitions of the remaining variables are provided in the first aspect or any one of the first through the nineteenth embodiments or any alternative embodiments described therein.

In a twenty-first embodiment, the present disclosure provides a compound according to the first aspect or any one of the first through the twentieth embodiments or a pharmaceutically acceptable salt thereof, wherein R¹¹, for each occurrence, is independently selected from F, —C(═O)CH₃, —C(═O)CH₂CH₃, —C(═O)cyclopropyl, —CH₃, —CH₂CH₃, —CH(CH₃)₂, —C(CH₃)₃, —CH₂C(CH₃)₃, —CH₂CH₂OCH₃, —CH₂CH₂CH₂OCH₃, —CH₂CH₂CH₂OCH₃, —CH₂CHF₂, —CH₂CH₂F, —CH₂-cyclopropyl, —CH₂-cyclobutyl, cyclopropyl, cyclobutyl and cyclopentyl,

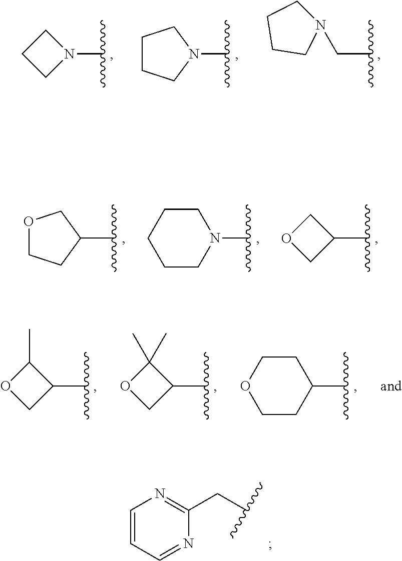

wherein said cyclopropyl, cyclobutyl, or cyclopentyl represented by R¹¹ is optionally substituted by one to two substituents independently selected from D, F, C₁₋₃alkoxy and C₁₋₃alkyl. The definitions of the remaining variables are provided in the first aspect or in any one of the first through the twentieth embodiments or any alternative embodiments described therein. In some embodiments, R¹¹ is cyclopropyl optionally substituted by one to two substituents independently selected from F and C₁₋₃alkyl.

In an alternative twenty-first embodiment, the present disclosure provides a compound according to the first aspect or any one of the first through the twentieth embodiments or a pharmaceutically acceptable salt thereof, wherein R¹¹, for each occurrence, is independently selected from F, —C(═O)CH₃, —C(═O)CH₂CH₃, —C(═O)cyclopropyl, —CH₃, —CH₂CH₃, —CH(CH₃)₂, —CH₂CH₂OCH₃, —CH₂CH₂CH₂OCH₃, cyclopropyl, and cyclobutyl; wherein said cyclopropyl represented by R¹¹ is optionally substituted by one to two substituents independently selected from F and C₁₋₃alkyl. The definitions of the remaining variables are provided in the first aspect or in any one of the first through the twentieth embodiments. In some embodiments, R¹¹ is cyclopropyl optionally substituted by one to two substituents independently selected from F and C₁₋₃alkyl or any alternative embodiments described therein.

In a twenty-second embodiment, the present disclosure provides a compound according to the first aspect or any one of the first through the twenty-first embodiments or a pharmaceutically acceptable salt thereof, wherein R⁹ is H or C₁₋₃alkyl and R¹⁰ is H, C₃₋₆cycloalkyl or C₁₋₃alkyl. The definitions of the remaining variables are provided in the first aspect or any one of the first through the twenty-first embodiments or any alternative embodiments described therein.

In an alternative twenty-second embodiment, the present disclosure provides a compound according to the first aspect or any one of the first through the twenty-first embodiments or a pharmaceutically acceptable salt thereof, wherein R⁹ and R¹⁰ are each independently H or C₁₋₃alkyl. The definitions of the remaining variables are provided in the first aspect or any one of the first through the twenty-first embodiments or any alternative embodiments described therein.

In a twenty-third embodiment, the present disclosure provides a compound according to the first aspect or any one of the first through the twenty-second embodiments or a pharmaceutically acceptable salt thereof, wherein R⁹ is H or —CH₃ and R¹⁰ is H, cyclopropyl or —CH₃. The definitions of the remaining variables are provided in the first aspect or any one of first through the twenty-second embodiments or any alternative embodiments described therein.

In an alternative twenty-third embodiment, the present disclosure provides a compound according to the first aspect or any one of the first through the twenty-second embodiments or a pharmaceutically acceptable salt thereof, wherein R⁹ and R¹⁰ are each independently H or —CH₃. The definitions of the remaining variables are provided in the first aspect or any one of the first through the twenty-second embodiments or any alternative embodiments described therein.

In a twenty-fourth embodiment, the present disclosure provides a compound according to the first aspect or any one of the first through the twenty-third embodiments or a pharmaceutically acceptable salt thereof, wherein B is phenyl, naphthalenyl, or 8 to 10 membered bicyclic heteroaryl; wherein said phenyl, naphthalenyl, and 8 to 10 membered bicyclic heteroaryl represented by B are optionally substituted by one to three R⁸. The definitions of the remaining variables are provided in the first aspect or any one of the first through the twenty-third embodiments or any alternative embodiments described therein.

In a twenty-fifth embodiment, the present disclosure provides a compound according to any one of the first aspect or any one of the first through the twenty-fourth embodiments or a pharmaceutically acceptable salt thereof, wherein B is a 9 or 10 membered bicyclic heteroaryl optionally substituted by one to three R⁸. The definitions of the remaining variables are provided in the first aspect or any one of the first through the twenty-fourth embodiments or any alternative embodiments described therein.

In an alternative twenty-fifth embodiment, the present disclosure provides a compound according to the first aspect or any one of the first through the twenty-fourth embodiments or a pharmaceutically acceptable salt thereof, wherein B is a 9 membered bicyclic heteroaryl optionally substituted by one to three R⁸. The definitions of the remaining variables are provided in the first aspect or any one of the first through the twenty-fourth embodiments or any alternative embodiments described therein.

In a twenty-sixth embodiment, the present disclosure provides a compound according to the first aspect or any one of the first through the twenty-third embodiments or a pharmaceutically acceptable salt thereof, wherein B is selected from a group consisting of phenyl, indazolyl, imidazopyridinyl, imidazopyridazinyl, benzotriazolyl, imidazopyrazinyl, benzooxazolyl, triazolopyridinyl, benzisothiazolyl, pyrazolopyridinyl, pyrazolopyrazinyl, pyrazolopyrimidinyl, thienopyridinyl, thienopyrimidinyl, benzothiazolyl, pyrrolopyridinyl, pyrrolopyrazinyl, benzofuranyl, benzothiophenyl, isoquinolinyl, pyrrolotriazinyl, thienopyridinyl, triazolopyridazinyl, benzooxadiazolyl, indolyl, indolin-2-onyl, furopyridine, benzoimidazolyl, benzothiadiazole, phthalazinyl and phthalazin-1-onyl, each of which is optionally substituted by one to three R⁸; or
B is 2H-pyrido[3,2-b][1,4]oxazin-3(4H)-onyl, each of which is optionally substituted with C₁₋₃alkyl. The definitions of the remaining variables are provided in the first aspect or any one of the first through the twenty-third embodiments or any alternative embodiments described therein.

In an alternative twenty-sixth embodiment, the present disclosure provides a compound according to the first aspect or any one of the first through the twenty-third embodiments or a pharmaceutically acceptable salt thereof, wherein B is selected from a group consisting of phenyl, indazolyl, imidazopyridinyl, imidazopyridazinyl, benzotriazolyl, imidazopyrazinyl, benzooxazolyl, triazolopyridinyl, benzisothiazolyl, pyrazolopyridinyl, thienopyridinyl, benzothiazolyl, pyrrolopyridinyl, pyrrolopyrazinyl, benzofuranyl, benzothiophenyl, thienopyridinyl, triazolopyridazinyl, benzooxadiazolyl, indolyl, indolin-2-onyl, furopyridine, benzoimidazolyl, benzothiadiazole, phthalazinyl and phthalazin-1-onyl, each of which is optionally substituted by one to three R⁸; or
B is 2H-pyrido[3,2-b][1,4]oxazin-3(4H)-onyl, each of which is optionally substituted with C₁₋₃alkyl. The definitions of the remaining variables are provided in the first aspect or any one of the first through the twenty-third embodiments or any alternative embodiments described therein.

In a twenty-seventh embodiment, the present disclosure provides a compound according to the twenty-sixth embodiment or a pharmaceutically acceptable salt thereof, wherein B is selected from:

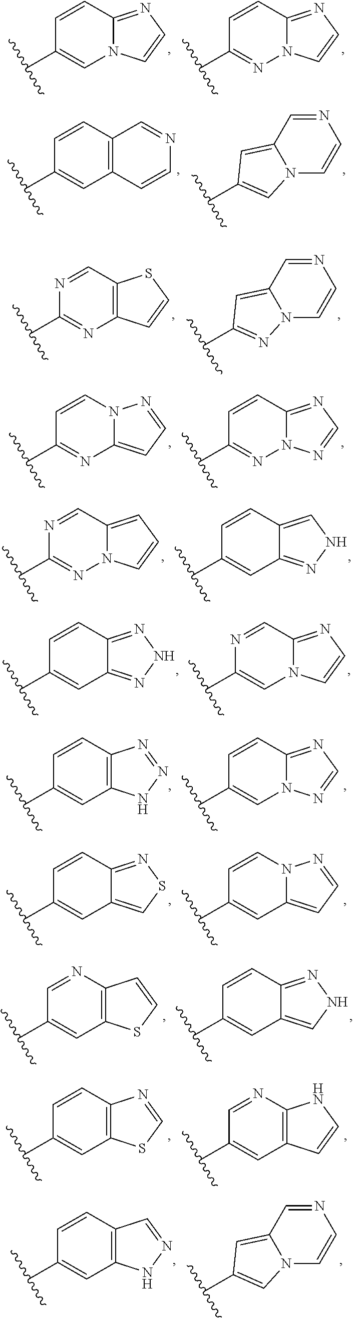

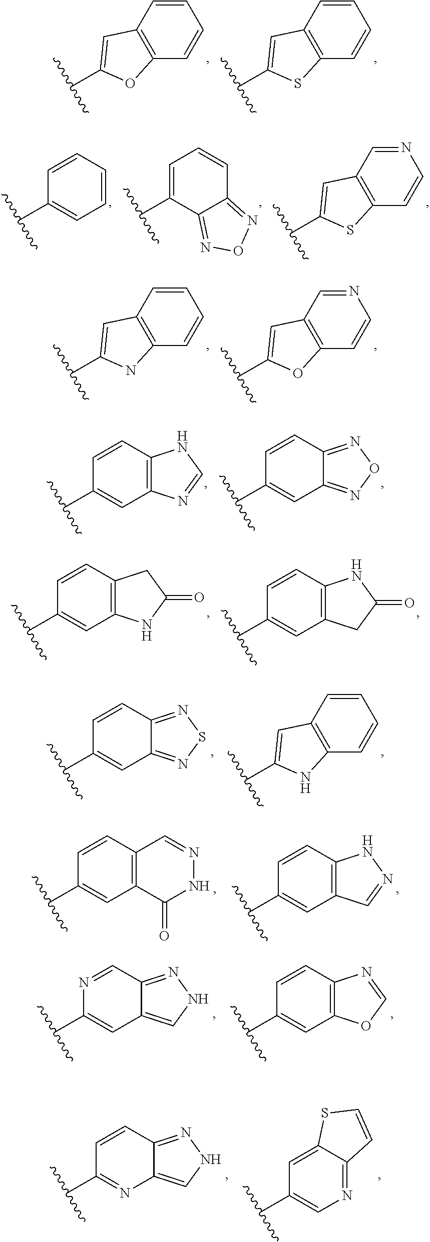

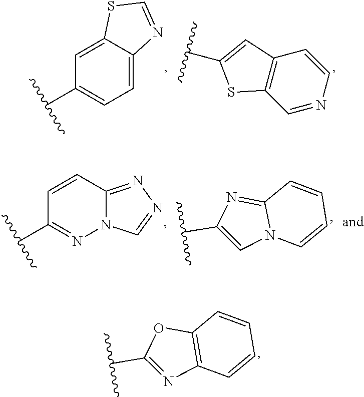

each of which is optionally substituted by one to three R⁸; or
B is

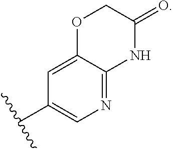 [SMILES: *c1cnc2c(c1)OCC(=O)N2]

The definitions of the remaining variables are provided in the twenty-sixth embodiments or any alternative embodiments described therein.

In an alternative twenty-seventh embodiment, the present disclosure provides a compound according to the twenty-sixth embodiment or a pharmaceutically acceptable salt thereof, wherein B is selected from:

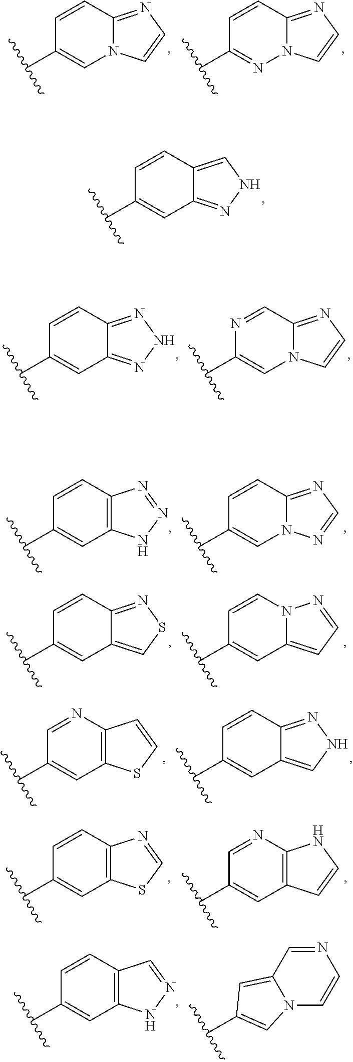

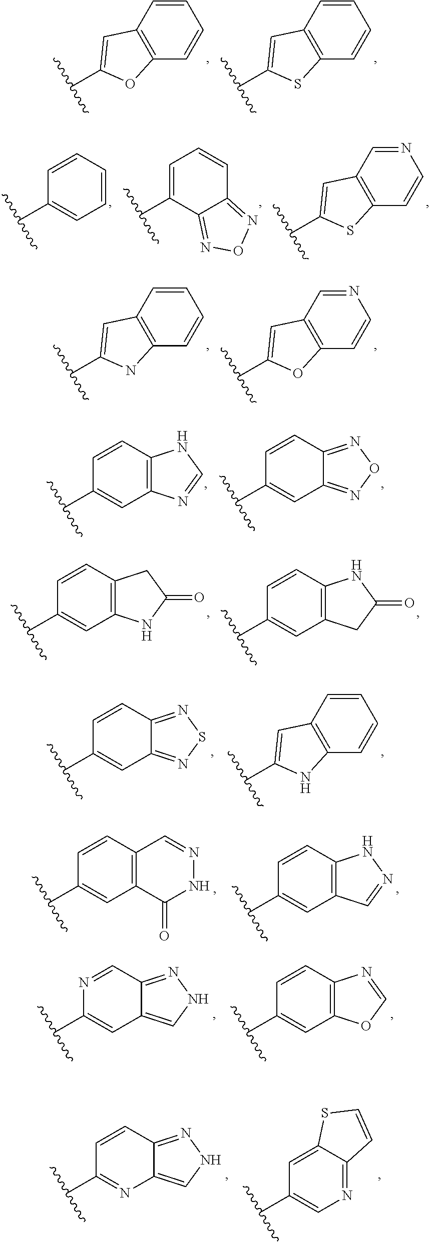

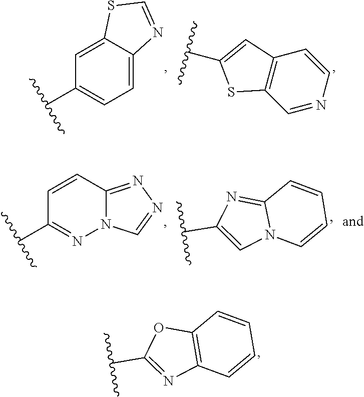

each of which is optionally substituted by one to three R⁸; or
B is

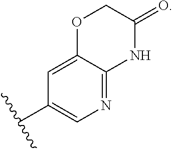 [SMILES: *c1cnc2c(c1)OCC(=O)N2]

The definitions of the remaining variables are provided in the twenty-sixth embodiments or any alternative embodiments described therein.

In a twenty-eighth embodiment, the present disclosure provides a compound according to the first aspect or any one of the first through the twenty-seventh embodiments or a pharmaceutically acceptable salt thereof, wherein R⁸ for each occurrence is halo, —CN, —OH, C₁₋₃alkyl, C₃₋₆cycloalkyl, C₁₋₂haloalkyl, or C₁₋₂alkoxy. The definitions of the remaining variables are provided in the first aspect or any one of the first through the twenty-seventh embodiments or any alternative embodiments described therein.

In an alternative twenty-eighth embodiment, the present disclosure provides a compound according to the first aspect or any one of the first through the twenty-seventh embodiments or a pharmaceutically acceptable salt thereof, wherein R⁸ for each occurrence is halo, —CN, —OH, C₁₋₃alkyl, C₁₋₂haloalkyl, or C₁₋₂alkoxy. The definitions of the remaining variables are provided in the first aspect or any one of the first through the twenty-seventh embodiments or any alternative embodiments described therein.

In a twenty-ninth embodiment, the present disclosure provides a compound according to the twenty-eighth embodiment or a pharmaceutically acceptable salt thereof, wherein R⁸ for each occurrence is independently selected from —F, —Cl, —Br, —CN, —CH₃, —CH₂CH₃, —CH(CH₃)₂, —CHF₂, —CF₃, —OH, —OCH₃, —OCH₂CH₃, and cyclopropyl. The definitions of the remaining variables are provided in the twenty-eighth embodiment or any alternative embodiments described therein.

In an alternative twenty-ninth embodiment, the present disclosure provides a compound according to the twenty-eighth embodiment or a pharmaceutically acceptable salt thereof, wherein R⁸ for each occurrence is independently selected from —F, —Cl, —CN, —CH₃, —CH₂CH₃, —CH(CH₃)₂, —CHF₂, —OH, —OCH₃, and —OCH₂CH₃. The definitions of the remaining variables are provided in the twenty-eighth embodiment or any alternative embodiments described therein.

In a thirtieth embodiment, the present disclosure provides a compound according to the first aspect or any one of the first through the twenty-ninth embodiments or a pharmaceutically acceptable salt thereof, wherein R^{8a} for each occurrence is —CH₃ or —CH₂CH₃; and Ra for each occurrence is —CH₃ or oxo. The definitions of the remaining variables are provided in the first aspect or any one of the first through the twenty-ninth embodiments or any alternative embodiments described therein.

In a thirty-first embodiment, the present disclosure provides a compound according to the first aspect or the first embodiment or a pharmaceutically acceptable salt thereof, wherein the compound is represented by the following formula:

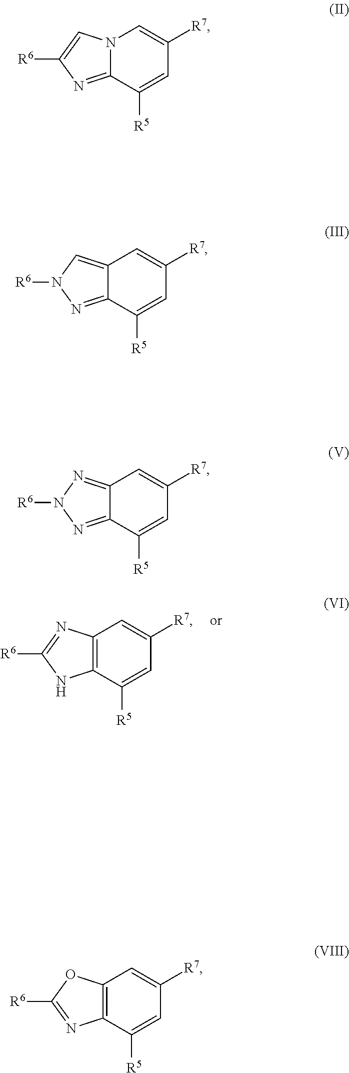

or a pharmaceutically acceptable salt thereof, wherein:
R⁵ is halo;
R⁶ is A, —NH—C(═O)-A, —C(═O)NH-A or —NH-A;
R⁷ is B;
A is 4 to 6 membered monocyclic saturated heterocyclyl, 6 to 10 membered bicyclic saturated fused or spiral heterocyclyl, or C₃₋₆cycloalkyl, wherein the 4 to 6 membered monocyclic saturated heterocyclyl and 6 to 10 membered bicyclic saturated fused or spiral heterocyclyl are each optionally substituted with 1 or 2 R¹¹, and the C₃₋₆cycloalkyl is substituted with —NR⁹R¹⁰ and is further optionally substituted with R¹¹;
R⁹ and R¹⁰ are each independently H or C₁₋₃alkyl;
Each R¹¹ is independently C₁₋₃alkyl or C₃₋₆cycloalkyl,
B is 9-membered bicyclic heteroaryl optionally substituted with 1 to 3 R⁸, wherein the 9-membered bicyclic heteroaryl has 2 to 4 N ring atoms; and
   Each R⁸ is independently C₁₋₃alkyl, C₁₋₃haloalkyl or C₁₋₃alkoxy.

In a thirty-second embodiment, the present disclosure provides a compound according to the thirty-first embodiment or a pharmaceutically acceptable salt thereof, wherein:
R⁵ is F;
A is cyclobutyl or Het, wherein Het is azetidinyl, piperidinyl, 3-azabicyclo[3.1.0]hexanyl, 2,8-diazaspiro[4.5]decanyl, or 2,7-azaspiro[3.5]nonanyl, each of which is optionally substituted with C₁₋₃alkyl or C₃₋₆cycloalkyl, and wherein cyclobutyl represented by A is optionally substituted with —NR⁹R¹⁰;
R⁹ and R¹⁰ are each H or —CH₃;
B is represented by the following formula:

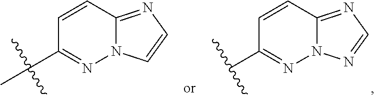 [SMILES: *c1ccc2nccn2n1 ; *c1ccc2ncnn2n1]

each of which is optionally substitute with 1 or 2 R⁸; and
each R⁸ is independently C₁₋₃alkyl, C₁₋₃haloalkyl or C₁₋₃alkoxy. The definitions of the remaining variables are provided in the thirty-first embodiment.

In a thirty-third embodiment, the present disclosure provides a compound according to the thirty-first or thirty-second embodiment or a pharmaceutically acceptable salt thereof, wherein A is represented by the following formula:

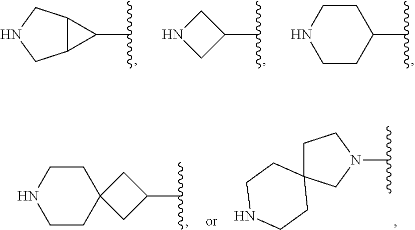 [SMILES: *C1C2CNCC12 ; *C1CNC1 ; *C1CCNCC1 ; *C1CC2(CCNCC2)C1 ; *N1CCC2(CCNCC2)C1]

each of which is optionally substituted with C₁₋₃alkyl. The definitions of the remaining variables are provided in the thirty-first or thirty-second embodiment.

In a thirty-fourth embodiment, the present disclosure provides a compound according to any one of the thirty-first, thirty-second and thirty-third embodiments or a pharmaceutically acceptable salt thereof, wherein A is represented by the following formula:

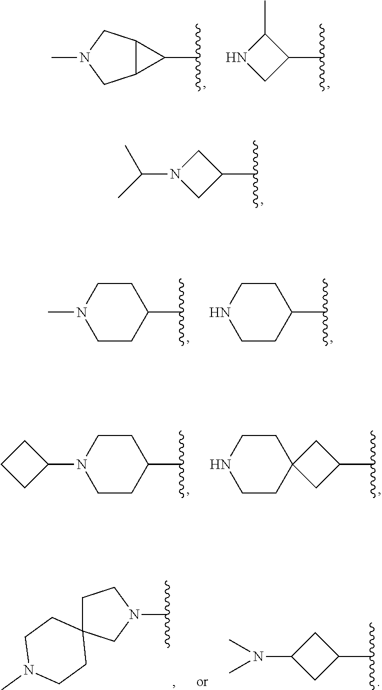

The definitions of the remaining variables are provided in the thirty-first, thirty-second or thirty-third embodiment.

In a thirty-fifth embodiment, the present disclosure provides a compound according to any one of the thirty-first through thirty-fourth embodiment or a pharmaceutically acceptable salt thereof, wherein B is represented by the following formula:

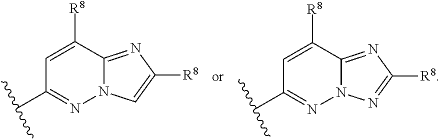

The definitions of the remaining variables are provided in the thirty-first, thirty-second, thirty-third or thirty-fourth embodiment.

In a thirty-sixth embodiment, the present disclosure provides a compound according to any one of the thirty-first through thirty-fifth embodiments or a pharmaceutically acceptable salt thereof, wherein each R⁸ is —CH₃, —CHF₂, CF₃, or —OCH₃. The definitions of the remaining variables are provided in the thirty-first, thirty-second, thirty-third, thirty-fourth or thirty-fifth embodiment.

In one embodiment, the present disclosure provides a compound selected from the compounds disclosed in examples and Table 1, a pharmaceutically acceptable salt or a stereoisomer thereof.

TABLE 1

| Cpd ID | Structure | Name |
|---|---|---|
| 1 | 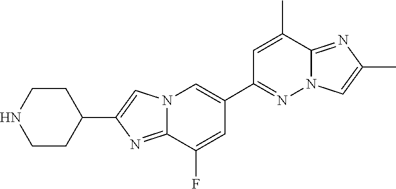 [SMILES: Cc1cn2nc(-c3cc(F)c4nc(C5CCNCC5)cn4c3)cc(C)c2n1] | 6-(8-fluoro-2-(piperidin- 4-yl)imidazo[1,2- a]pyridin-6-yl)-2,8- dimethylimidazo[1,2- b]pyridazine |
| 2 | 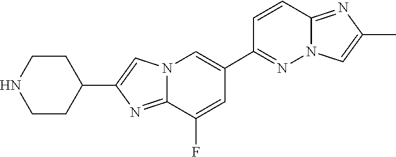 [SMILES: Cc1cn2nc(-c3cc(F)c4nc(C5CCNCC5)cn4c3)ccc2n1] | 6-(8-fluoro-2-(piperidin- 4-yl)imidazo[1,2- a]pyridin-6-yl)-2- methylimidazo[1,2- b]pyridazine |
| 3 | 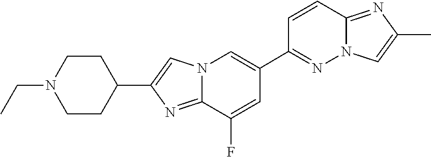 [SMILES: CCN1CCC(c2cn3cc(-c4ccc5nc(C)cn5n4)cc(F)c3n2)CC1] | 6-(2-(1-ethylpiperidin-4- yl)-8-fluoroimidazo[1,2- a]pyridin-6-yl)-2- methylimidazo[1,2- b]pyridazine |
| 4 | 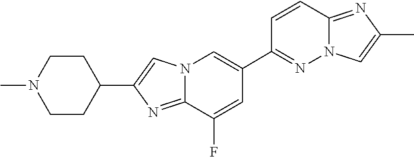 | 6-(8-fluoro-2-(1- methylpiperidin-4- yl)imidazo[1,2- a]pyridin-6-yl)-2- methylimidazo[1,2- b]pyridazine |
| 5 | 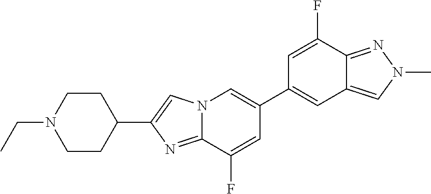 [SMILES: CCN1CCC(c2cn3cc(-c4cc(F)c5nn(C)cc5c4)cc(F)c3n2)CC1] | 5-(2-(1-ethylpiperidin-4- yl)-8-fluoroimidazo[1,2- a]pyridin-6-yl)-7-fluoro- 2-methyl-2H-indazole |
| 6 | 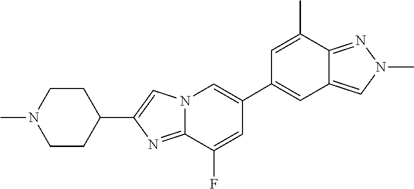 | 5-(8-fluoro-2-(1- methylpiperidin-4- yl)imidazo[1,2- a]pyridin-6-yl)-2,7- dimethyl-2H-indazole |
| 7 | 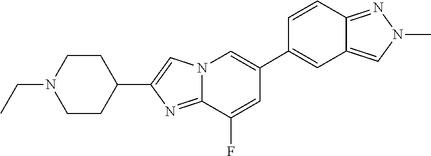 [SMILES: CCN1CCC(c2cn3cc(-c4ccc5nn(C)cc5c4)cc(F)c3n2)CC1] | 5-(2-(1-ethylpiperidin-4- yl)-8-fluoroimidazo[1,2- a]pyridin-6-yl)-2- methyl-2H-indazole |
| 8 | 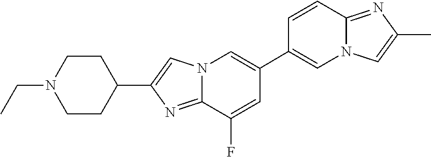 [SMILES: CCN1CCC(c2cn3cc(-c4ccc5nc(C)cn5c4)cc(F)c3n2)CC1] | 2-(1-ethylpiperidin-4- yl)-8-fluoro-2′-methyl- 6,6′-biimidazo[1,2- a]pyridine |
| 9 | 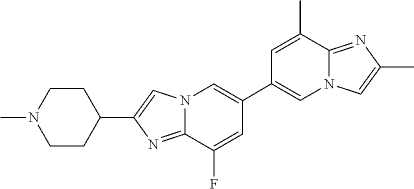 | 8-fluoro-2′,8′-dimethyl- 2-(1-methylpiperidin-4- yl)-6,6′-biimidazo[1,2- a]pyridine |
| 10 | 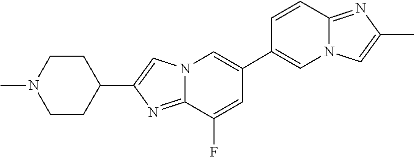 | 8-fluoro-2′-methyl-2-(1- methylpiperidin-4-yl)- 6,6′-biimidazo[1,2- a]pyridine |
| 11 | 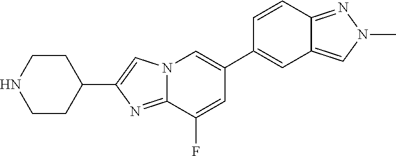 [SMILES: Cn1cc2cc(-c3cc(F)c4nc(C5CCNCC5)cn4c3)ccc2n1] | 5-(8-fluoro-2-(piperidin- 4-yl)imidazo[1,2- a]pyridin-6-yl)-2- methyl-2H-indazole |
| 12 | 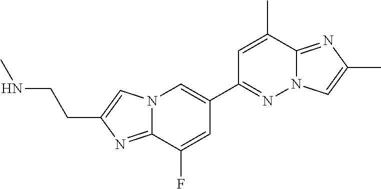 | 2-(6-(2,8- dimethylimidazo[1,2- b]pyridazin-6-yl)-8- fluoroimidazo[1,2- a]pyridin-2-yl)-N- methylethan-1-amine |
| 13 | 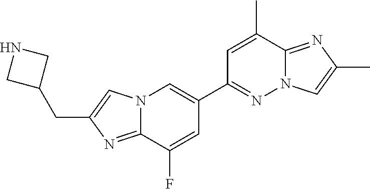 [SMILES: Cc1cn2nc(-c3cc(F)c4nc(CC5CNC5)cn4c3)cc(C)c2n1] | 6-(2-(azetidin-3- ylmethyl)-8- fluoroimidazo[1,2- a]pyridin-6-yl)-2,8- dimethylimidazo[1,2- b]pyridazine |
| 14 | 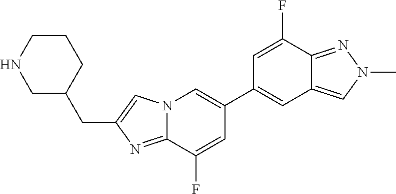 [SMILES: Cn1cc2cc(-c3cc(F)c4nc(CC5CCCNC5)cn4c3)cc(F)c2n1] | 7-fluoro-5-(8-fluoro-2- (piperidin-3- ylmethyl)imidazo[1,2- a]pyridin-6-yl)-2- methyl-2H-indazole |
| 15 | 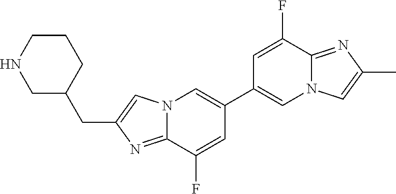 [SMILES: Cc1cn2cc(-c3cc(F)c4nc(CC5CCCNC5)cn4c3)cc(F)c2n1] | 8,8′-difluoro-2-methyl- 2′-(piperidin-3- ylmethyl)-6,6′- biimidazo[1,2- a]pyridine |
| 16 | 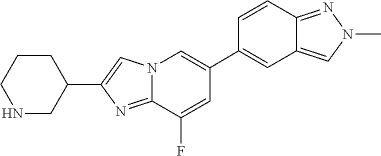 [SMILES: Cn1cc2cc(-c3cc(F)c4nc(C5CCCNC5)cn4c3)ccc2n1] | 5-(8-fluoro-2-(piperidin- 3-yl)imidazo[1,2- a]pyridin-6-yl)-2- methyl-2H-indazole |
| 17 | 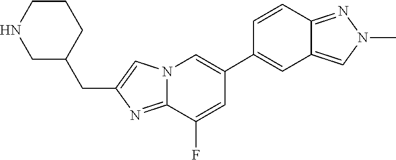 [SMILES: Cn1cc2cc(-c3cc(F)c4nc(CC5CCCNC5)cn4c3)ccc2n1] | 5-(8-fluoro-2-(piperidin- 3-ylmethyl)imidazo[1,2- a]pyridin-6-yl)-2- methyl-2H-indazole |
| 18 | 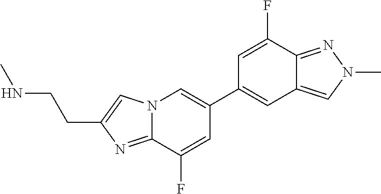 | 2-(8-fluoro-6-(7-fluoro- 2-methyl-2H-indazol-5- yl)imidazo[1,2- a]pyridin-2-yl)-N- methylethan-1-amine |
| 19 | 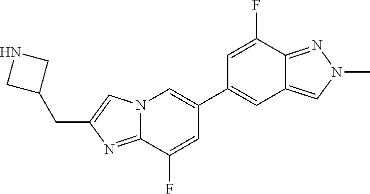 [SMILES: Cn1cc2cc(-c3cc(F)c4nc(CC5CNC5)cn4c3)cc(F)c2n1] | 5-(2-(azetidin-3- ylmethyl)-8- fluoroimidazo[1,2- a]pyridin-6-yl)-7-fluoro- 2-methyl-2H-indazole |
| 20 | 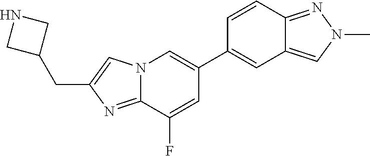 [SMILES: Cn1cc2cc(-c3cc(F)c4nc(CC5CNC5)cn4c3)ccc2n1] | 5-(2-(azetidin-3- ylmethyl)-8- fluoroimidazo[1,2- a]pyridin-6-yl)-2- methyl-2H-indazole |
| 21 | 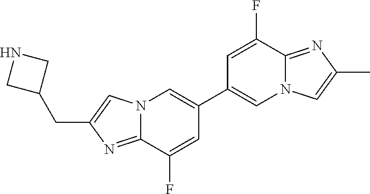 [SMILES: Cc1cn2cc(-c3cc(F)c4nc(CC5CNC5)cn4c3)cc(F)c2n1] | 2-(azetidin-3-ylmethyl)- 8,8′-difluoro-2′-methyl- 6,6′-biimidazo[1,2- a]pyridine |
| 22 | 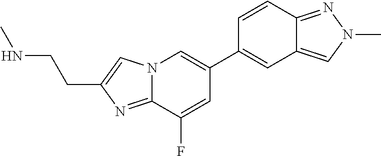 | 2-(8-fluoro-6-(2-methyl- 2H-indazol-5- yl)imidazo[1,2- a]pyridin-2-yl)-N- methylethan-1-amine |
| 23 | 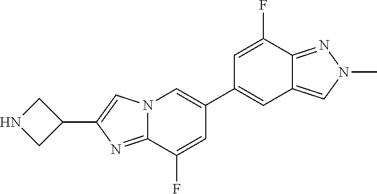 [SMILES: Cn1cc2cc(-c3cc(F)c4nc(C5CNC5)cn4c3)cc(F)c2n1] | 5-(2-(azetidin-3-yl)-8- fluoroimidazo[1,2- a]pyridin-6-yl)-7-fluoro- 2-methyl-2H-indazole |
| 24 | 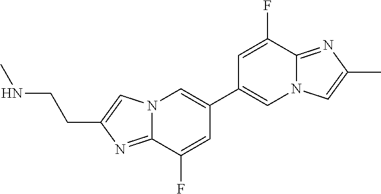 | 2-(8,8′-difluoro-2′- methyl-[6,6′- biimidazo[1,2- a]pyridin]-2-yl)-N- methylethan-1-amine |
| 25 | 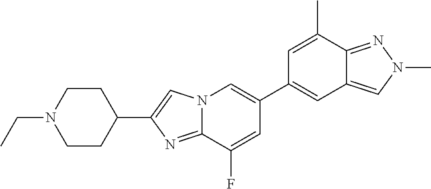 [SMILES: CCN1CCC(c2cn3cc(-c4cc(C)c5nn(C)cc5c4)cc(F)c3n2)CC1] | 5-(2-(1-ethylpiperidin-4- yl)-8-fluoroimidazo[1,2- a]pyridin-6-yl)-2,7- dimethyl-2H-indazole |
| 26 | 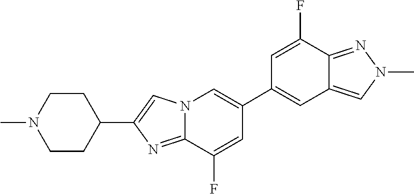 | 7-fluoro-5-(8-fluoro-2- (1-methylpiperidin-4- yl)imidazo[1,2- a]pyridin-6-yl)-2- methyl-2H-indazole |
| 27 | 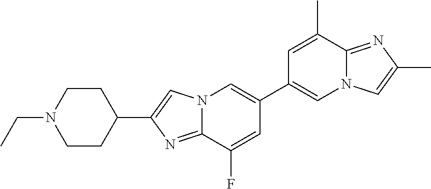 [SMILES: CCN1CCC(c2cn3cc(-c4cc(C)c5nc(C)cn5c4)cc(F)c3n2)CC1] | 2-(1-ethylpiperidin-4- yl)-8-fluoro-2′,8′- dimethyl-6,6′- biimidazo[1,2- a]pyridine |
| 28 | 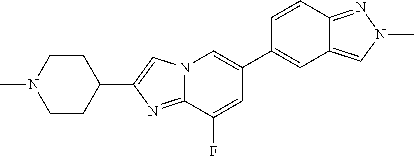 | 5-(8-fluoro-2-(1- methylpiperidin-4- yl)imidazo[1,2- a]pyridin-6-yl)-2- methyl-2H-indazole |
| 29 | 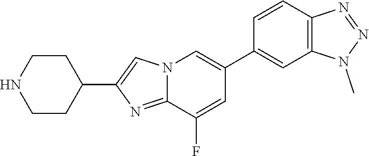 [SMILES: Cn1nnc2ccc(-c3cc(F)c4nc(C5CCNCC5)cn4c3)cc21] | 6-(8-fluoro-2-(piperidin- 4-yl)imidazo[1,2- a]pyridin-6-yl)-1- methyl-1H- benzo[d][1,2,3]triazole |
| 30 | 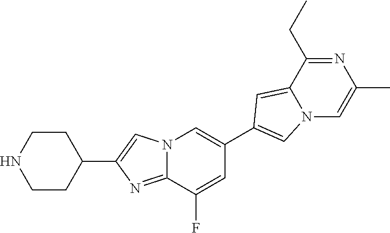 [SMILES: CCc1nc(C)cn2cc(-c3cc(F)c4nc(C5CCNCC5)cn4c3)cc12] | 6-(1-ethyl-3- methylpyrrolo[1,2- a]pyrazin-7-yl)-8- fluoro-2-(piperidin-4- yl)imidazo[1,2- a]pyridine |
| 31 | 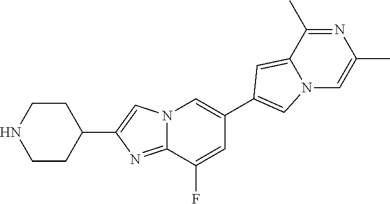 [SMILES: Cc1cn2cc(-c3cc(F)c4nc(C5CCNCC5)cn4c3)cc2c(C)n1] | 6-(1,3- dimethylpyrrolo[1,2- a]pyrazin-7-yl)-8- fluoro-2-(piperidin-4- yl)imidazo[1,2- a]pyridine |
| 32 | 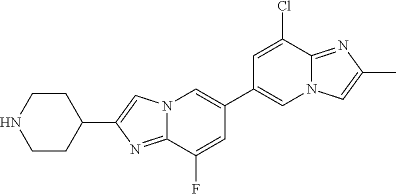 [SMILES: Cc1cn2cc(-c3cc(F)c4nc(C5CCNCC5)cn4c3)cc(Cl)c2n1] | 8-chloro-8′-fluoro-2- methyl-2′-(piperidin-4- yl)-6,6′-biimidazo[1,2- a]pyridine |
| 33 | 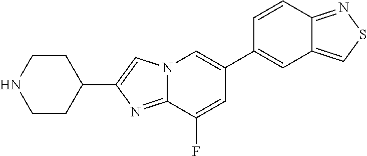 [SMILES: Fc1cc(-c2ccc3nscc3c2)cn2cc(C3CCNCC3)nc12] | 5-(8-fluoro-2-(piperidin- 4-yl)imidazo[1,2- a]pyridin-6- yl)benzo[c]isothiazole |
| 34 | 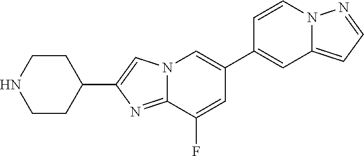 [SMILES: Fc1cc(-c2ccn3nccc3c2)cn2cc(C3CCNCC3)nc12] | 8-fluoro-2-(piperidin-4- yl)-6-(pyrazolo[1,5- a]pyridin-5- yl)imidazo[1,2- a]pyridine |
| 35 | 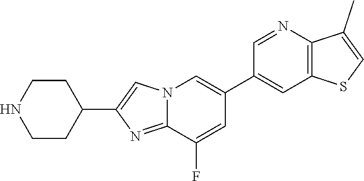 [SMILES: Cc1csc2cc(-c3cc(F)c4nc(C5CCNCC5)cn4c3)cnc12] | 6-(8-fluoro-2-(piperidin- 4-yl)imidazo[1,2- a]pyridin-6-yl)-3- methylthieno[3,2- b]pyridine |
| 36 | 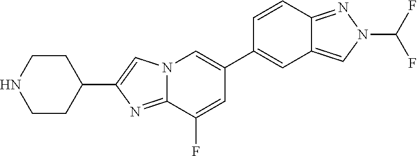 [SMILES: Fc1cc(-c2ccc3nn(C(F)F)cc3c2)cn2cc(C3CCNCC3)nc12] | 2-(difluoromethyl)-5-(8- fluoro-2-(piperidin-4- yl)imidazo[1,2- a]pyridin-6-yl)-2H- indazole |
| 37 | 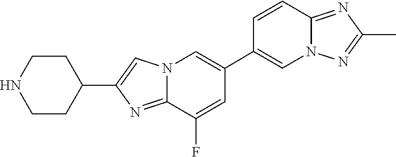 [SMILES: Cc1nc2ccc(-c3cc(F)c4nc(C5CCNCC5)cn4c3)cn2n1] | 6-(8-fluoro-2-(piperidin- 4-yl)imidazo[1,2- a]pyridin-6-yl)-2- methyl- [1,2,4]triazolo[1,5- a]pyridine |
| 38 | 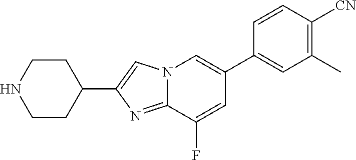 [SMILES: Cc1cc(-c2cc(F)c3nc(C4CCNCC4)cn3c2)ccc1C#N] | 4-(8-fluoro-2-(piperidin- 4-yl)imidazo[1,2- a]pyridin-6-yl)-2- methylbenzonitrile |
| 39 | 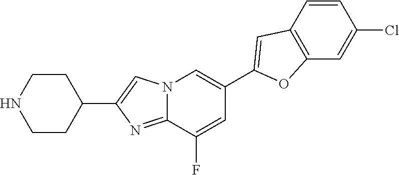 [SMILES: Fc1cc(-c2cc3ccc(Cl)cc3o2)cn2cc(C3CCNCC3)nc12] | 6-(6-chlorobenzofuran- 2-yl)-8-fluoro-2- (piperidin-4- yl)imidazo[1,2- a]pyridine |
| 40 | 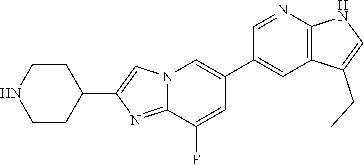 [SMILES: CCc1c[nH]c2ncc(-c3cc(F)c4nc(C5CCNCC5)cn4c3)cc12] | 6-(3-ethyl-1H- pyrrolo[2,3-b]pyridin-5- yl)-8-fluoro-2- (piperidin-4- yl)imidazo[1,2- a]pyridine |
| 41 | 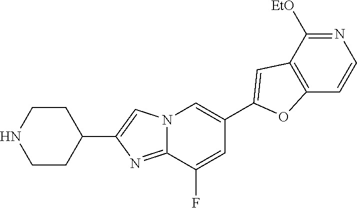 [SMILES: CCOc1nccc2oc(-c3cc(F)c4nc(C5CCNCC5)cn4c3)cc12] | 4-ethoxy-2-(8-fluoro-2- (piperidin-4- yl)imidazo[1,2- a]pyridin-6-yl)furo[3,2- c]pyridine |
| 42 | 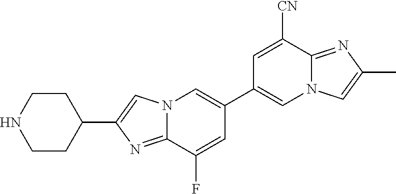 [SMILES: Cc1cn2cc(-c3cc(F)c4nc(C5CCNCC5)cn4c3)cc(C#N)c2n1] | 8′-fluoro-2-methyl-2′- (piperidin-4-yl)-[6,6′- biimidazo[1,2- a]pyridine]-8- carbonitrile |
| 43 | 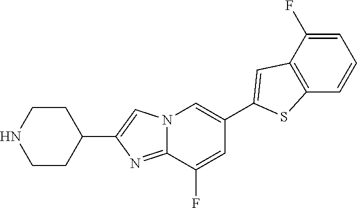 [SMILES: Fc1cccc2sc(-c3cc(F)c4nc(C5CCNCC5)cn4c3)cc12] | 8-fluoro-6-(4- fluorobenzo[b]thiophen- 2-yl)-2-(piperidin-4- yl)imidazo[1,2- a]pyridine |
| 44 | 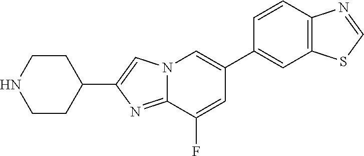 [SMILES: Fc1cc(-c2ccc3ncsc3c2)cn2cc(C3CCNCC3)nc12] | 6-(8-fluoro-2-(piperidin- 4-yl)imidazo[1,2- a]pyridin-6- yl)benzo[d]thiazole |
| 45 | 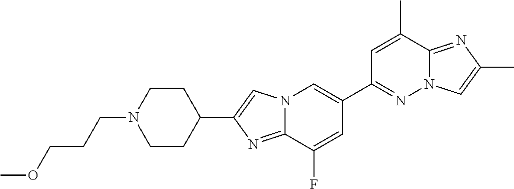 | 6-(8-fluoro-2-(1-(3- methoxypropyl)piperidin- 4-yl)imidazo[1,2- a]pyridin-6-yl)-2,8- dimethylimidazo[1,2- b]pyridazine |
| 46 | 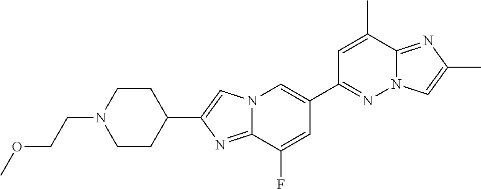 | 6-(8-fluoro-2-(1-(2- methoxyethyl)piperidin- 4-yl)imidazo[1,2- a]pyridin-6-yl)-2,8- dimethylimidazo[1,2- b]pyridazine |
| 47 | 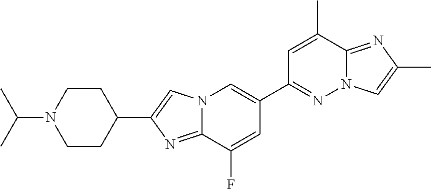 [SMILES: Cc1cn2nc(-c3cc(F)c4nc(C5CCN(C(C)C)CC5)cn4c3)cc(C)c2n1] | 6-(8-fluoro-2-(1- isopropylpiperidin-4- yl)imidazo[1,2- a]pyridin-6-yl)-2,8- dimethylimidazo[1,2- b]pyridazine |
| 48 | 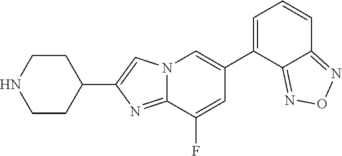 [SMILES: Fc1cc(-c2cccc3nonc23)cn2cc(C3CCNCC3)nc12] | 4-(8-fluoro-2-(piperidin- 4-yl)imidazo[1,2- a]pyridin-6- yl)benzo[c][1,2,5]oxadia zole |
| 49 | 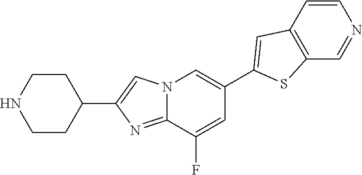 [SMILES: Fc1cc(-c2cc3ccncc3s2)cn2cc(C3CCNCC3)nc12] | 2-(8-fluoro-2-(piperidin- 4-yl)imidazo[1,2- a]pyridin-6- yl)thieno[3,2-c]pyridine |
| 50 | 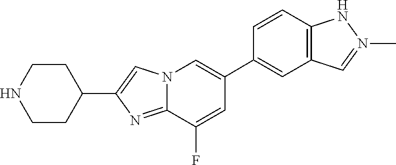 | 5-(8-fluoro-2-(piperidin- 4-yl)imidazo[1,2- a]pyridin-6-yl)-2- methyl-1H- benzo[d]imidazole |
| 51 | 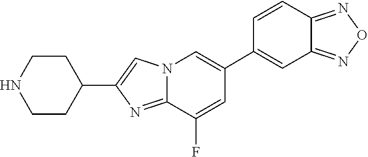 [SMILES: Fc1cc(-c2ccc3nonc3c2)cn2cc(C3CCNCC3)nc12] | 5-(8-fluoro-2-(piperidin- 4-yl)imidazo[1,2- a]pyridin-6- yl)benzo[c][1,2,5]oxadia zole |
| 52 | 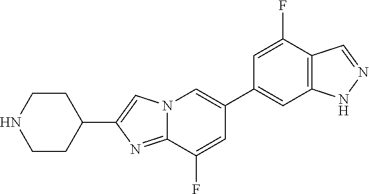 [SMILES: Fc1cc(-c2cc(F)c3nc(C4CCNCC4)cn3c2)cc2[nH]ncc12] | 4-fluoro-6-(8-fluoro-2- (piperidin-4- yl)imidazo[1,2- a]pyridin-6-yl)-1H- indazole |
| 53 | 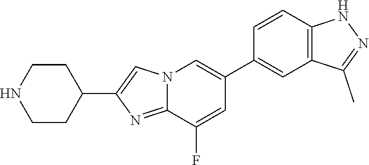 [SMILES: Cc1n[nH]c2ccc(-c3cc(F)c4nc(C5CCNCC5)cn4c3)cc12] | 5-(8-fluoro-2-(piperidin- 4-yl)imidazo[1,2- a]pyridin-6-yl)-3- methyl-1H-indazole |
| 54 | 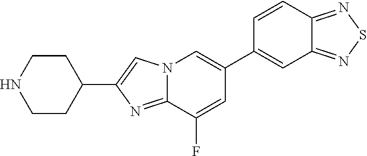 [SMILES: Fc1cc(-c2ccc3nsnc3c2)cn2cc(C3CCNCC3)nc12] | 5-(8-fluoro-2-(piperidin- 4-yl)imidazo[1,2- a]pyridin-6- yl)benzo[c][1,2,5]thiadi azole |
| 55 | 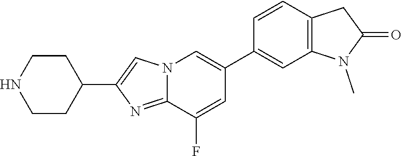 [SMILES: CN1C(=O)Cc2ccc(-c3cc(F)c4nc(C5CCNCC5)cn4c3)cc21] | 6-(8-fluoro-2-(piperidin- 4-yl)imidazo[1,2- a]pyridin-6-yl)-1- methylindolin-2-one |
| 56 | 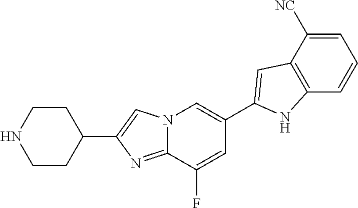 [SMILES: N#Cc1cccc2[nH]c(-c3cc(F)c4nc(C5CCNCC5)cn4c3)cc12] | 2-(8-fluoro-2-(piperidin- 4-yl)imidazo[1,2- a]pyridin-6-yl)-1H- indole-4-carbonitrile |
| 57 | 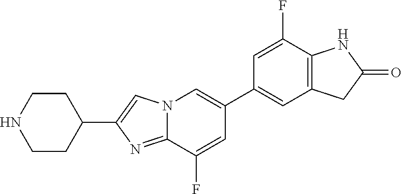 [SMILES: O=C1Cc2cc(-c3cc(F)c4nc(C5CCNCC5)cn4c3)cc(F)c2N1] | 7-fluoro-5-(8-fluoro-2- (piperidin-4- yl)imidazo[1,2- a]pyridin-6-yl)indolin-2- one |
| 58 | 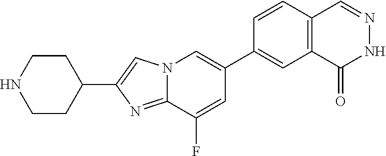 [SMILES: O=c1[nH]ncc2ccc(-c3cc(F)c4nc(C5CCNCC5)cn4c3)cc12] | 7-(8-fluoro-2-(piperidin- 4-yl)imidazo[1,2- a]pyridin-6- yl)phthalazin-1(2H)-one |
| 59 | 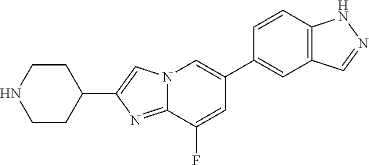 [SMILES: Fc1cc(-c2ccc3[nH]ncc3c2)cn2cc(C3CCNCC3)nc12] | 5-(8-fluoro-2-(piperidin- 4-yl)imidazo[1,2- a]pyridin-6-yl)-1H- indazole |
| 60 | 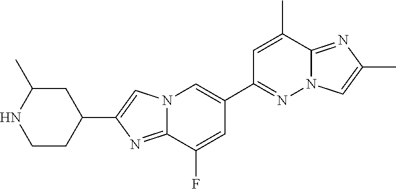 [SMILES: Cc1cn2nc(-c3cc(F)c4nc(C5CCNC(C)C5)cn4c3)cc(C)c2n1] | 6-(8-fluoro-2-(2- methylpiperidin-4- yl)imidazo[1,2- a]pyridin-6-yl)-2,8- dimethylimidazo[1,2- b]pyridazine |
| 61 | 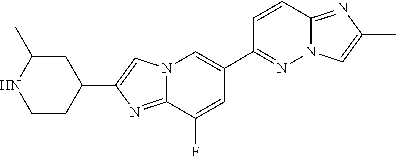 [SMILES: Cc1cn2nc(-c3cc(F)c4nc(C5CCNC(C)C5)cn4c3)ccc2n1] | 6-(8-fluoro-2-(2- methylpiperidin-4- yl)imidazo[1,2- a]pyridin-6-yl)-2- methylimidazo[1,2- b]pyridazine |
| 62 | 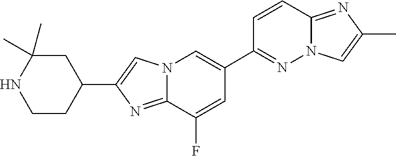 | 6-(2-(2,2- dimethylpiperidin-4-yl)- 8-fluoroimidazo[1,2- a]pyridin-6-yl)-2- methylimidazo[1,2- b]pyridazine |
| 63 | 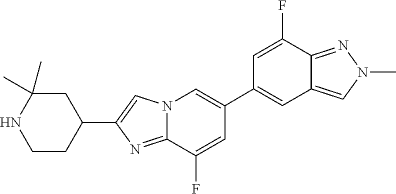 | 5-(2-(2,2- dimethylpiperidin-4-yl)- 8-fluoroimidazo[1,2- a]pyridin-6-yl)-7-fluoro- 2-methyl-2H-indazole |
| 64 | 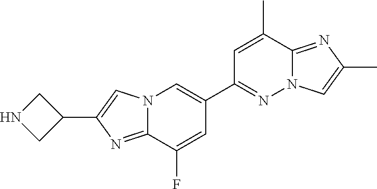 [SMILES: Cc1cn2nc(-c3cc(F)c4nc(C5CNC5)cn4c3)cc(C)c2n1] | 6-(2-(azetidin-3-yl)-8- fluoroimidazo[1,2- a]pyridin-6-yl)-2,8- dimethylimidazo[1,2- b]pyridazine |
| 65 | 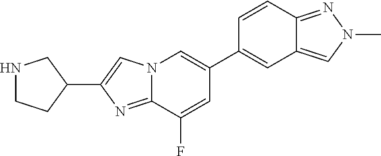 [SMILES: Cn1cc2cc(-c3cc(F)c4nc(C5CCNC5)cn4c3)ccc2n1] | 5-(8-fluoro-2- (pyrrolidin-3- yl)imidazo[1,2- a]pyridin-6-yl)-2- methyl-2H-indazole |
| 66 | 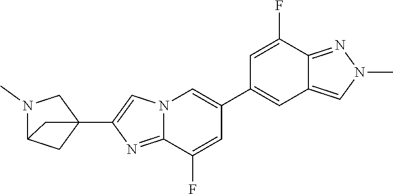 | 4-(8-fluoro-6-(7-fluoro- 2-methyl-2H-indazol-5- yl)imidazo[1,2- a]pyridin-2-yl)-2- methyl-2- azabicyclo[2.1.1]]hexane |
| 67 | 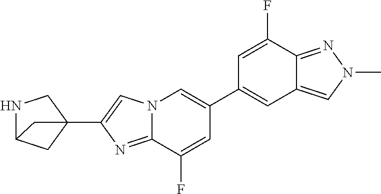 [SMILES: Cn1cc2cc(-c3cc(F)c4nc(C56CNC(C5)C6)cn4c3)cc(F)c2n1] | 4-(8-fluoro-6-(7-fluoro- 2-methyl-2H-indazol-5- yl)imidazo[1,2- a]pyridin-2-yl)-2- azabicyclo[2.1.1]]hexane |
| 68 | 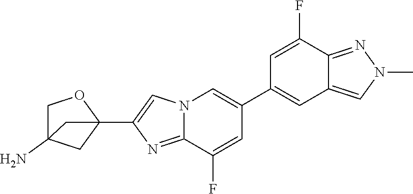 [SMILES: Cn1cc2cc(-c3cc(F)c4nc(C56CC(N)(CO5)C6)cn4c3)cc(F)c2n1] | 1-(8-fluoro-6-(7-fluoro- 2-methyl-2H-indazol-5- yl)imidazo[1,2- a]pyridin-2-yl)-2- oxabicyclo[2.1.1]]hexan- 4-amine |
| 69 | 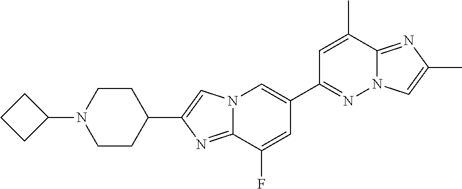 [SMILES: Cc1cn2nc(-c3cc(F)c4nc(C5CCN(C6CCC6)CC5)cn4c3)cc(C)c2n1] | 6-(2-(1- cyclobutylpiperidin-4- yl)-8-fluoroimidazo[1,2- a]pyridin-6-yl)-2,8- dimethylimidazo[1,2- b]pyridazine |
| 70* | 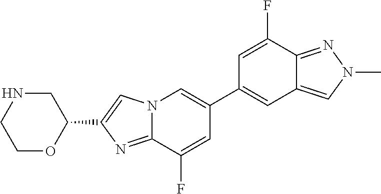 [SMILES: Cn1cc2cc(-c3cc(F)c4nc([C@H]5CNCCO5)cn4c3)cc(F)c2n1] | (R)-2-(8-fluoro-6-(7- fluoro-2-methyl-2H- indazol-5- yl)imidazo[1,2- a]pyridin-2- yl)morpholine |
| 71* | 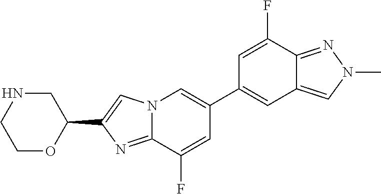 [SMILES: Cn1cc2cc(-c3cc(F)c4nc([C@@H]5CNCCO5)cn4c3)cc(F)c2n1] | (S)-2-(8-fluoro-6-(7- fluoro-2-methyl-2H- indazol-5- yl)imidazo[1,2- a]pyridin-2- yl)morpholine |
| 72 | 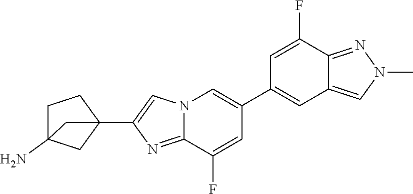 [SMILES: Cn1cc2cc(-c3cc(F)c4nc(C56CCC(N)(C5)C6)cn4c3)cc(F)c2n1] | 4-(8-fluoro-6-(7-fluoro- 2-methyl-2H-indazol-5- yl)imidazo[1,2- a]pyridin-2- yl)bicyclo[2.1.1]hexan- 1-amine |
| 73 | 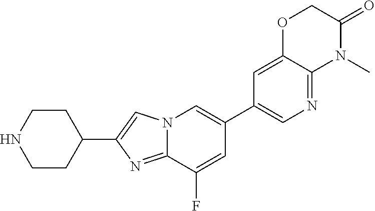 [SMILES: CN1C(=O)COc2cc(-c3cc(F)c4nc(C5CCNCC5)cn4c3)cnc21] | 7-(8-fluoro-2-(piperidin- 4-yl)imidazo[1,2- a]pyridin-6-yl)-4- methyl-2H-pyrido[3,2- b][1,4]oxazin-3(4H)-one |
| 74 | 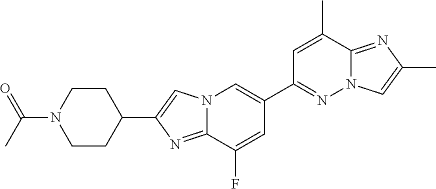 [SMILES: CC(=O)N1CCC(c2cn3cc(-c4cc(C)c5nc(C)cn5n4)cc(F)c3n2)CC1] | 1-(4-(6-(2,8- dimethylimidazo[1,2- b]pyridazin-6-yl)-8- fluoroimidazo[1,2- a]pyridin-2-yl)piperidin- 1-yl)ethan-1-one |
| 75 | 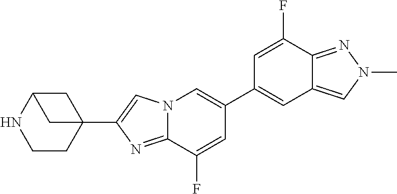 [SMILES: Cn1cc2cc(-c3cc(F)c4nc(C56CCNC(C5)C6)cn4c3)cc(F)c2n1] | 5-(8-fluoro-6-(7-fluoro- 2-methyl-2H-indazol-5- yl)imidazo[1,2- a]pyridin-2-yl)-2- azabicyclo[3.1.1]]heptane |
| 76 | 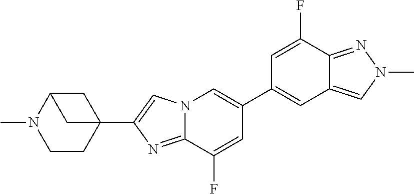 | 5-(8-fluoro-6-(7-fluoro- 2-methyl-2H-indazol-5- yl)imidazo[1,2- a]pyridin-2-yl)-2- methyl-2- azabicyclo[3.1.1]heptane |
| 77 | 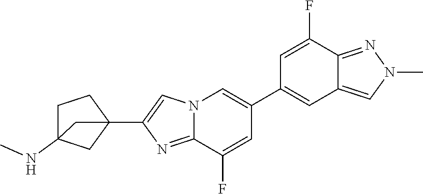 | 4-(8-fluoro-6-(7-fluoro- 2-methyl-2H-indazol-5- yl)imidazo[1,2- a]pyridin-2-yl)-N- methylbicyclo[2.1.1] hexan-1-amine |
| 78 | 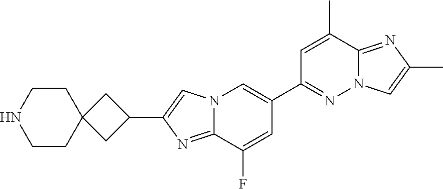 [SMILES: Cc1cn2nc(-c3cc(F)c4nc(C5CC6(CCNCC6)C5)cn4c3)cc(C)c2n1] | 6-(8-fluoro-2-(7- azaspiro[3.5]nonan-2- yl)imidazo[1,2- a]pyridin-6-yl)-2,8- dimethylimidazo[1,2- b]pyridazine |
| 79 | 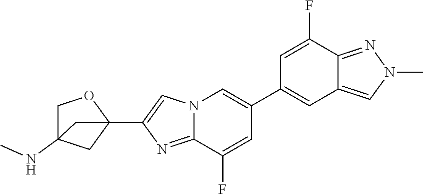 | 1-(8-fluoro-6-(7-fluoro- 2-methyl-2H-indazol-5- yl)imidazo[1,2- a]pyridin-2-yl)-N- methyl-2- oxabicyclo[2.1.1]]hexan- 4-amine |
| 80 | 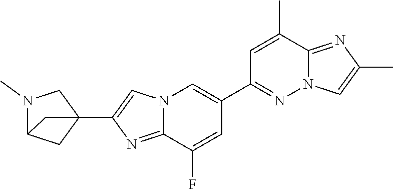 | 4-(6-(2,8- dimethylimidazo[1,2- b]pyridazin-6-yl)-8- fluoroimidazo[1,2- a]pyridin-2-yl)-2- methyl-2- azabicyclo[2.1.1]]hexane |
| 81 | 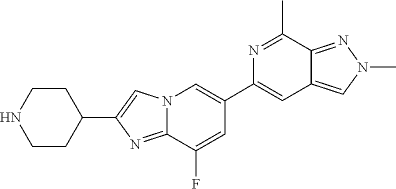 [SMILES: Cc1nc(-c2cc(F)c3nc(C4CCNCC4)cn3c2)cc2cn(C)nc12] | 5-(8-fluoro-2-(piperidin- 4-yl)imidazo[1,2- a]pyridin-6-yl)-2,7- dimethyl-2H- pyrazolo[3,4-c]pyridine |
| 82 | 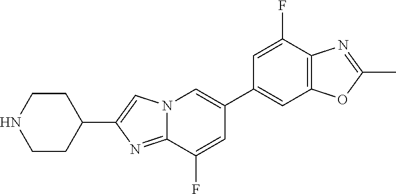 [SMILES: Cc1nc2c(F)cc(-c3cc(F)c4nc(C5CCNCC5)cn4c3)cc2o1] | 4-fluoro-6-(8-fluoro-2- (piperidin-4- yl)imidazo[1,2- a]pyridin-6-yl)-2- methylbenzo[d]oxazole |
| 83 | 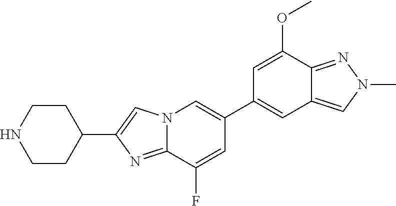 [SMILES: COc1cc(-c2cc(F)c3nc(C4CCNCC4)cn3c2)cc2cn(C)nc12] | 5-(8-fluoro-2-(piperidin- 4-yl)imidazo[1,2- a]pyridin-6-yl)-7- methoxy-2-methyl-2H- indazole |
| 84 | 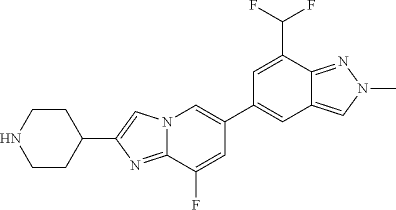 [SMILES: Cn1cc2cc(-c3cc(F)c4nc(C5CCNCC5)cn4c3)cc(C(F)F)c2n1] | 7-(difluoromethyl)-5-(8- fluoro-2-(piperidin-4- yl)imidazo[1,2- a]pyridin-6-yl)-2- methyl-2H-indazole |
| 85 | 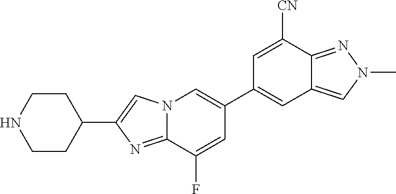 [SMILES: Cn1cc2cc(-c3cc(F)c4nc(C5CCNCC5)cn4c3)cc(C#N)c2n1] | 5-(8-fluoro-2-(piperidin- 4-yl)imidazo[1,2- a]pyridin-6-yl)-2- methyl-2H-indazole-7- carbonitrile |
| 86 | 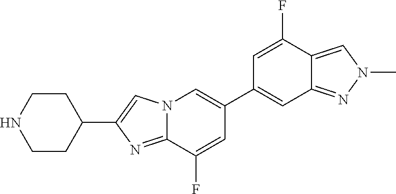 [SMILES: Cn1cc2c(F)cc(-c3cc(F)c4nc(C5CCNCC5)cn4c3)cc2n1] | 4-fluoro-6-(8-fluoro-2- (piperidin-4- yl)imidazo[1,2- a]pyridin-6-yl)-2- methyl-2H-indazole |
| 87 | 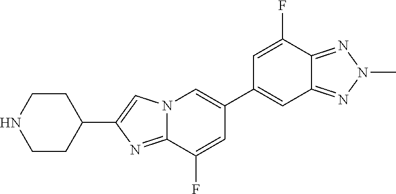 [SMILES: Cn1nc2cc(-c3cc(F)c4nc(C5CCNCC5)cn4c3)cc(F)c2n1] | 4-fluoro-6-(8-fluoro-2- (piperidin-4- yl)imidazo[1,2- a]pyridin-6-yl)-2- methyl-2H- benzo[d][1,2,3]triazole |
| 88 | 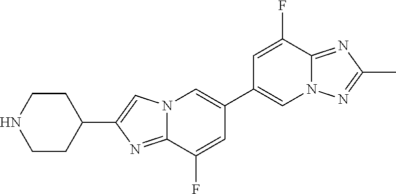 [SMILES: Cc1nc2c(F)cc(-c3cc(F)c4nc(C5CCNCC5)cn4c3)cn2n1] | 8-fluoro-6-(8-fluoro-2- (piperidin-4- yl)imidazo[1,2- a]pyridin-6-yl)-2- methyl- [1,2,4]triazolo[1,5- a]pyridine |
| 89 | 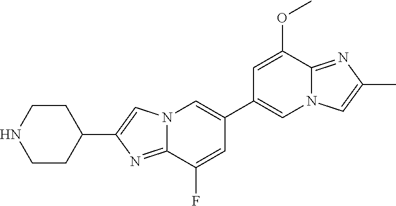 [SMILES: COc1cc(-c2cc(F)c3nc(C4CCNCC4)cn3c2)cn2cc(C)nc12] | 8-fluoro-8′-methoxy-2′- methyl-2-(piperidin-4- yl)-6,6′-biimidazo[1,2- a]pyridine |
| 90 | 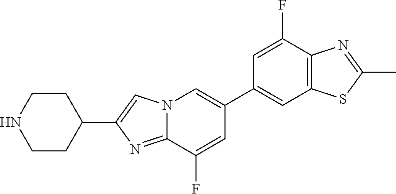 [SMILES: Cc1nc2c(F)cc(-c3cc(F)c4nc(C5CCNCC5)cn4c3)cc2s1] | 4-fluoro-6-(8-fluoro-2- (piperidin-4- yl)imidazo[1,2- a]pyridin-6-yl)-2- methylbenzo[d]thiazole |
| 91 | 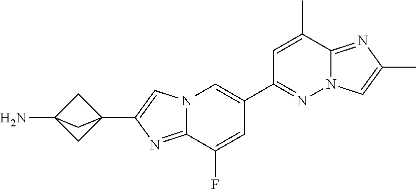 [SMILES: Cc1cn2nc(-c3cc(F)c4nc(C56CC(N)(C5)C6)cn4c3)cc(C)c2n1] | 3-(6-(2,8- dimethylimidazo[1,2- b]pyridazin-6-yl)-8- fluoroimidazo[1,2- a]pyridin-2- yl)bicyclo[1.1.1]]pentan- 1-amine |
| 92 | 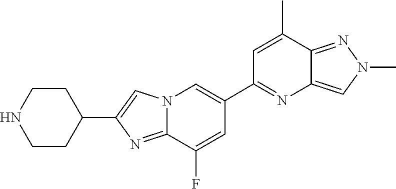 [SMILES: Cc1cc(-c2cc(F)c3nc(C4CCNCC4)cn3c2)nc2cn(C)nc12] | 5-(8-fluoro-2-(piperidin- 4-yl)imidazo[1,2- a]pyridin-6-yl)-2,7- dimethyl-2H- pyrazolo[4,3-b]pyridine |
| 93 | 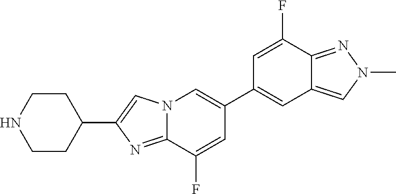 [SMILES: Cn1cc2cc(-c3cc(F)c4nc(C5CCNCC5)cn4c3)cc(F)c2n1] | 7-fluoro-5-(8-fluoro-2- (piperidin-4- yl)imidazo[1,2- a]pyridin-6-yl)-2- methyl-2H-indazole |
| 94 | 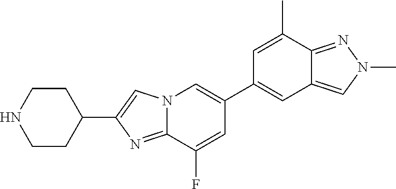 [SMILES: Cc1cc(-c2cc(F)c3nc(C4CCNCC4)cn3c2)cc2cn(C)nc12] | 5-(8-fluoro-2-(piperidin- 4-yl)imidazo[1,2- a]pyridin-6-yl)-2,7- dimethyl-2H-indazole |
| 95 | 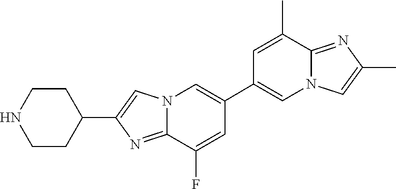 [SMILES: Cc1cn2cc(-c3cc(F)c4nc(C5CCNCC5)cn4c3)cc(C)c2n1] | 8-fluoro-2′,8′-dimethyl- 2-(piperidin-4-yl)-6,6′- biimidazo[1,2- a]pyridine |
| 96 | 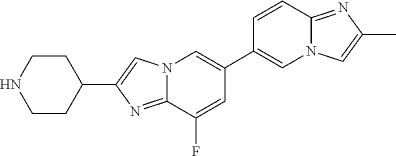 [SMILES: Cc1cn2cc(-c3cc(F)c4nc(C5CCNCC5)cn4c3)ccc2n1] | 8-fluoro-2′-methyl-2- (piperidin-4-yl)-6,6′- biimidazo[1,2- a]pyridine |
| 97 | 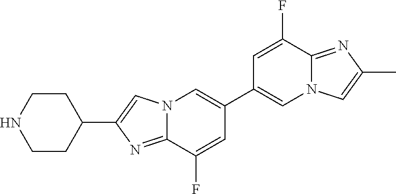 [SMILES: Cc1cn2cc(-c3cc(F)c4nc(C5CCNCC5)cn4c3)cc(F)c2n1] | 8,8′-difluoro-2-methyl- 2′-(piperidin-4-yl)-6,6′- biimidazo[1,2- a]pyridine |
| 98 | 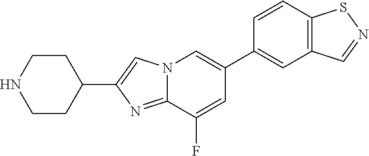 [SMILES: Fc1cc(-c2ccc3sncc3c2)cn2cc(C3CCNCC3)nc12] | 5-(8-fluoro-2-(piperidin- 4-yl)imidazo[1,2- a]pyridin-6- yl)benzo[d]isothiazole |
| 99 | 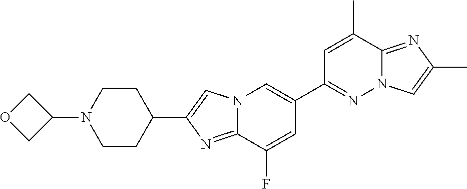 [SMILES: Cc1cn2nc(-c3cc(F)c4nc(C5CCN(C6COC6)CC5)cn4c3)cc(C)c2n1] | 6-(8-fluoro-2-(1- (oxetan-3-yl)piperidin- 4-yl)imidazo[1,2- a]pyridin-6-yl)-2,8- dimethylimidazo[1,2- b]pyridazine |
| 100 | 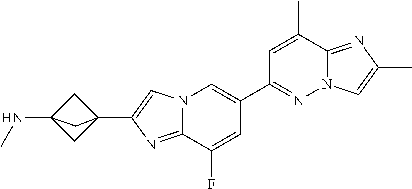 | 3-(6-(2,8- dimethylimidazo[1,2- b]pyridazin-6-yl)-8- fluoroimidazo[1,2- a]pyridin-2-yl)-N- methylbicyclo[1.1.1] pentan-1-amine |
| 101 | 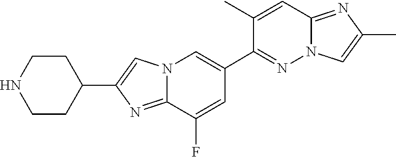 [SMILES: Cc1cn2nc(-c3cc(F)c4nc(C5CCNCC5)cn4c3)c(C)cc2n1] | 6-(8-fluoro-2-(piperidin- 4-yl)imidazo[1,2- a]pyridin-6-yl)-2,7- dimethylimidazo[1,2- b]pyridazine |
| 102 | 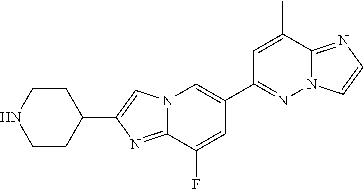 [SMILES: Cc1cc(-c2cc(F)c3nc(C4CCNCC4)cn3c2)nn2ccnc12] | 6-(8-fluoro-2-(piperidin- 4-yl)imidazo[1,2- a]pyridin-6-yl)-8- methylimidazo[1,2- b]pyridazine |
| 103 | 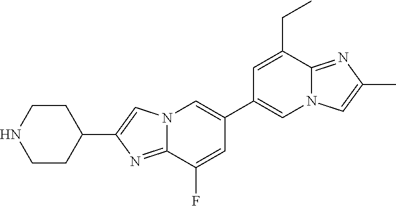 [SMILES: CCc1cc(-c2cc(F)c3nc(C4CCNCC4)cn3c2)cn2cc(C)nc12] | 8-ethyl-8′-fluoro-2- methyl-2′-(piperidin-4- yl)-6,6′-biimidazo[1,2- a]pyridine |
| 104 | 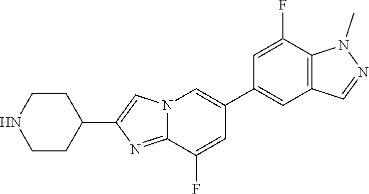 [SMILES: Cn1ncc2cc(-c3cc(F)c4nc(C5CCNCC5)cn4c3)cc(F)c21] | 7-fluoro-5-(8-fluoro-2- (piperidin-4- yl)imidazo[1,2- a]pyridin-6-yl)-1- methyl-1H-indazole |
| 105 | 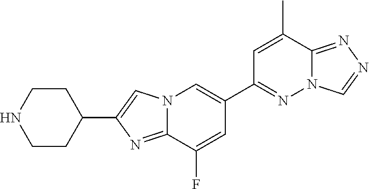 [SMILES: Cc1cc(-c2cc(F)c3nc(C4CCNCC4)cn3c2)nn2cnnc12] | 6-(8-fluoro-2-(piperidin- 4-yl)imidazo[1,2- a]pyridin-6-yl)-8- methyl- [1,2,4]triazolo[4,3- b]pyridazine |
| 106 | 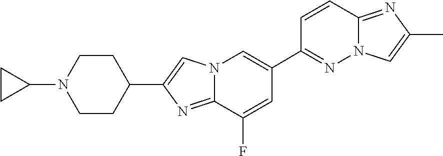 [SMILES: Cc1cn2nc(-c3cc(F)c4nc(C5CCN(C6CC6)CC5)cn4c3)ccc2n1] | 6-(2-(1- cyclopropylpiperidin-4- yl)-8-fluoroimidazo[1,2- a]pyridin-6-yl)-2- methylimidazo[1,2- b]pyridazine |
| 107 | 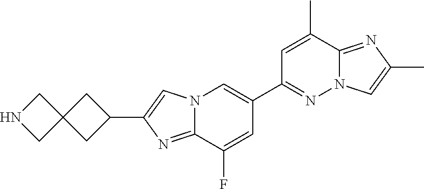 [SMILES: Cc1cn2nc(-c3cc(F)c4nc(C5CC6(CNC6)C5)cn4c3)cc(C)c2n1] | 6-(8-fluoro-2-(2- azaspiro[3.3]]heptan-6- yl)imidazo[1,2- a]pyridin-6-yl)-2,8- dimethylimidazo[1,2- b]pyridazine |
| 108 | 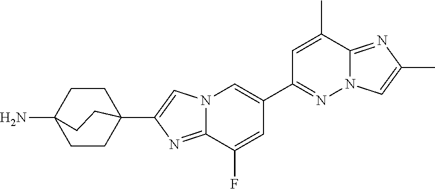 [SMILES: Cc1cn2nc(-c3cc(F)c4nc(C56CCC(N)(CC5)CC6)cn4c3)cc(C)c2n1] | 4-(6-(2,8- dimethylimidazo[1,2- b]pyridazin-6-yl)-8- fluoroimidazo[1,2- a]pyridin-2- yl)bicyclo[2.2.2]octan- 1-amine |
| 109 | 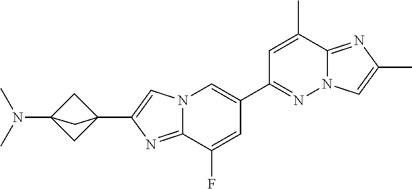 | 3-(6-(2,8- dimethylimidazo[1,2- b]pyridazin-6-yl)-8- fluoroimidazo[1,2- a]pyridin-2-yl)-N,N- dimethylbicyclo[1.1.1] pentan-1-amine |
| 110 | 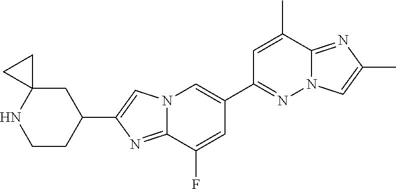 [SMILES: Cc1cn2nc(-c3cc(F)c4nc(C5CCNC6(CC6)C5)cn4c3)cc(C)c2n1] | 6-(8-fluoro-2-(4- azaspiro[2.5]octan-7- yl)imidazo[1,2- a]pyridin-6-yl)-2,8- dimethylimidazo[1,2- b]pyridazine |
| 111 | 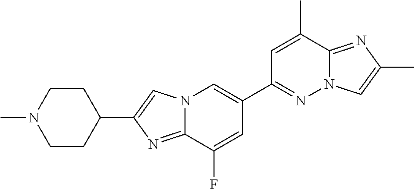 | 6-(8-fluoro-2-(1- methylpiperidin-4- yl)imidazo[1,2- a]pyridin-6-yl)-2,8- dimethylimidazo[1,2- b]pyridazine |
| 112 | 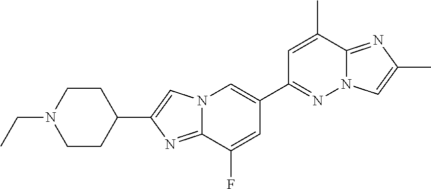 [SMILES: CCN1CCC(c2cn3cc(-c4cc(C)c5nc(C)cn5n4)cc(F)c3n2)CC1] | 6-(2-(1-ethylpiperidin-4- yl)-8-fluoroimidazo[1,2- a]pyridin-6-yl)-2,8- dimethylimidazo[1,2- b]pyridazine |
| 113 | 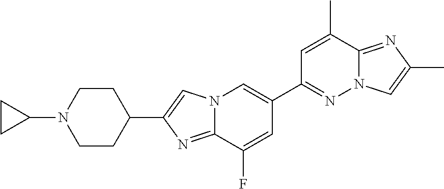 [SMILES: Cc1cn2nc(-c3cc(F)c4nc(C5CCN(C6CC6)CC5)cn4c3)cc(C)c2n1] | 6-(2-(1- cyclopropylpiperidin-4- yl)-8-fluoroimidazo[1,2- a]pyridin-6-yl)-2,8- dimethylimidazo[1,2- b]pyridazine |
| 114* | 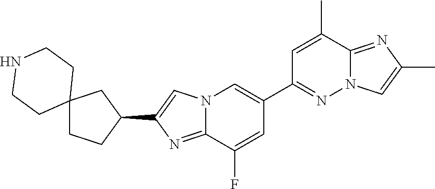 [SMILES: Cc1cn2nc(-c3cc(F)c4nc([C@H]5CCC6(CCNCC6)C5)cn4c3)cc(C)c2n1] | (S)-2-(6-(2,8- dimethylimidazo[1,2- b]pyridazin-6-yl)-8- fluoroimidazo[1,2- a]pyridin-2-yl)-8- azaspiro[4.5]decane |
| 115* | 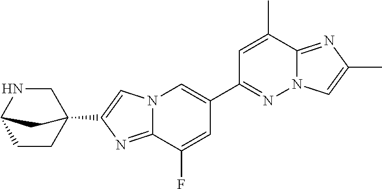 [SMILES: Cc1cn2nc(-c3cc(F)c4nc([C@]56CC[C@@H](C5)NC6)cn4c3)cc(C)c2n1] | 6-(2-((1S,4R)-2- azabicyclo[2.2.1]]heptan- 4-yl)-8- fluoroimidazo[1,2- a]pyridin-6-yl)-2,8- dimethylimidazo[1,2- b]pyridazine |
| 116* | 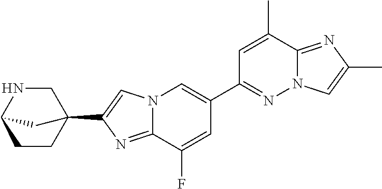 [SMILES: Cc1cn2nc(-c3cc(F)c4nc([C@@]56CC[C@H](C5)NC6)cn4c3)cc(C)c2n1] | 6-(2-((1R,4S)-2- azabicyclo[2.2.1]heptan- 4-yl)-8- fluoroimidazo[1,2- a]pyridin-6-yl)-2,8- dimethylimidazo[1,2- b]pyridazine |
| 117* | 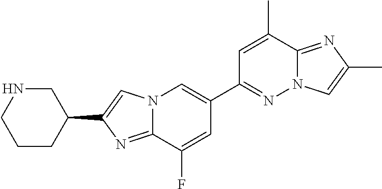 [SMILES: Cc1cn2nc(-c3cc(F)c4nc([C@H]5CCCNC5)cn4c3)cc(C)c2n1] | (S)-6-(8-fluoro-2- (piperidin-3- yl)imidazo[1,2- a]pyridin-6-yl)-2,8- dimethylimidazo[1,2- b]pyridazine |
| 118* | 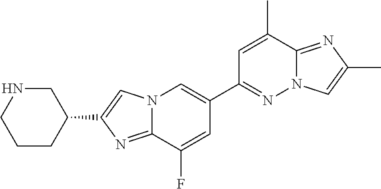 [SMILES: Cc1cn2nc(-c3cc(F)c4nc([C@@H]5CCCNC5)cn4c3)cc(C)c2n1] | (R)-6-(8-fluoro-2- (piperidin-3- yl)imidazo[1,2- a]pyridin-6-yl)-2,8- dimethylimidazo[1,2- b]pyridazine |
| 119* | 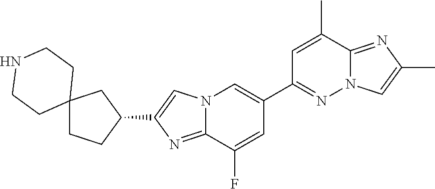 [SMILES: Cc1cn2nc(-c3cc(F)c4nc([C@@H]5CCC6(CCNCC6)C5)cn4c3)cc(C)c2n1] | (R)-2-(6-(2,8- dimethylimidazo[1,2- b]pyridazin-6-yl)-8- fluoroimidazo[1,2- a]pyridin-2-yl)-8- azaspiro[4.5]decane |
| 120 | 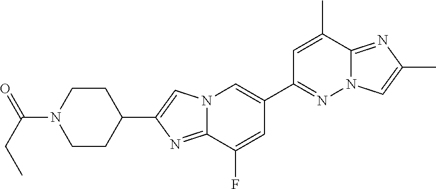 [SMILES: CCC(=O)N1CCC(c2cn3cc(-c4cc(C)c5nc(C)cn5n4)cc(F)c3n2)CC1] | 1-(4-(6-(2,8- dimethylimidazo[1,2- b]pyridazin-6-yl)-8- fluoroimidazo[1,2- a]pyridin-2-yl)piperidin- 1-yl)propan-1-one |
| 121 | 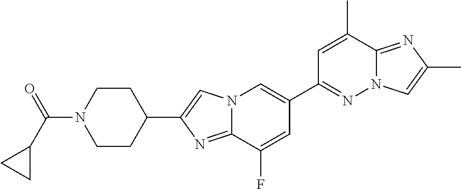 [SMILES: Cc1cn2nc(-c3cc(F)c4nc(C5CCN(C(=O)C6CC6)CC5)cn4c3)cc(C)c2n1] | cyclopropyl(4-(6-(2,8- dimethylimidazo[1,2- b]pyridazin-6-yl)-8- fluoroimidazo[1,2- a]pyridin-2-yl)piperidin- 1-yl)methanone |
| 122 | 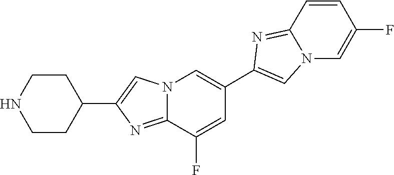 [SMILES: Fc1ccc2nc(-c3cc(F)c4nc(C5CCNCC5)cn4c3)cn2c1] | 6,8′-difluoro-2′- (piperidin-4-yl)-2,6′- biimidazo[1,2- a]pyridine |
| 123 | 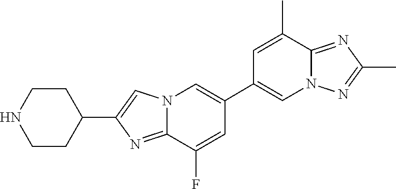 [SMILES: Cc1nc2c(C)cc(-c3cc(F)c4nc(C5CCNCC5)cn4c3)cn2n1] | 6-(8-fluoro-2-(piperidin- 4-yl)imidazo[1,2- a]pyridin-6-yl)-2,8- dimethyl- [1,2,4]triazolo[1,5- a]pyridine |
| 124 | 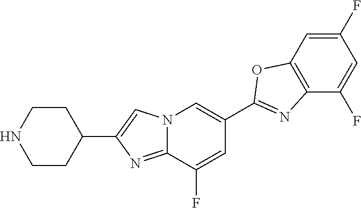 [SMILES: Fc1cc(F)c2nc(-c3cc(F)c4nc(C5CCNCC5)cn4c3)oc2c1] | 4,6-difluoro-2-(8-fluoro- 2-(piperidin-4- yl)imidazo[1,2- a]pyridin-6- yl)benzo[d]oxazole |
| 125 | 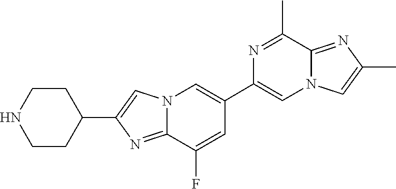 [SMILES: Cc1cn2cc(-c3cc(F)c4nc(C5CCNCC5)cn4c3)nc(C)c2n1] | 6-(8-fluoro-2-(piperidin- 4-yl)imidazo[1,2- a]pyridin-6-yl)-2,8- dimethylimidazo[1,2- a]pyrazine |
| 158 | 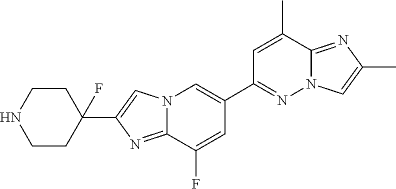 [SMILES: Cc1cn2nc(-c3cc(F)c4nc(C5(F)CCNCC5)cn4c3)cc(C)c2n1] | 6-(8-fluoro-2-(4- fluoropiperidin-4- yl)imidazo[1,2- a]pyridin-6-yl)-2,8- dimethylimidazo[1,2- b]pyridazine |
| 159 | 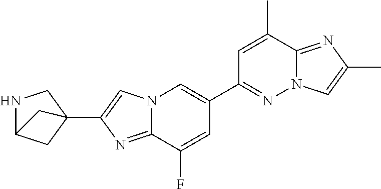 [SMILES: Cc1cn2nc(-c3cc(F)c4nc(C56CNC(C5)C6)cn4c3)cc(C)c2n1] | 4-(6-(2,8- dimethylimidazo[1,2- b]pyridazin-6-yl)-8- fluoroimidazo[1,2- a]pyridin-2-yl)-2- azabicyclo[2.1.1]]hexane |
| 160 | 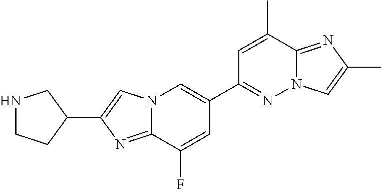 [SMILES: Cc1cn2nc(-c3cc(F)c4nc(C5CCNC5)cn4c3)cc(C)c2n1] | 6-(8-fluoro-2- (pyrrolidin-3- yl)imidazo[1,2- a]pyridin-6-yl)-2,8- dimethylimidazo[1,2- b]pyridazine |
| 161 | 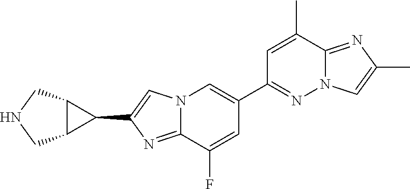 [SMILES: Cc1cn2nc(-c3cc(F)c4nc([C@H]5[C@@H]6CNC[C@@H]65)cn4c3)cc(C)c2n1] | 6-(2-((1R,5S,6r)-3- azabicyclo[3.1.0]hexan- 6-yl)-8- fluoroimidazo[1,2- a]pyridin-6-yl)-2,8- dimethylimidazo[1,2- b]pyridazine |
| 162 | 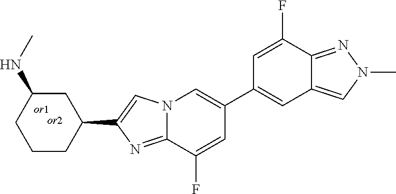 [SMILES: CN[C@@H]1CCC[C@H](c2cn3cc(-c4cc(F)c5nn(C)cc5c4)cc(F)c3n2)C1] | rel-(1R,3S)-3-(8-fluoro- 6-(7-fluoro-2-methyl- 2H-indazol-5- yl)imidazo[1,2- a]pyridin-2-yl)-N- methylcyclohexan-1- amine |
| 163 | 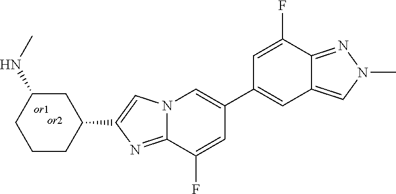 [SMILES: CN[C@H]1CCC[C@@H](c2cn3cc(-c4cc(F)c5nn(C)cc5c4)cc(F)c3n2)C1] | rel-(1R,3S)-3-(8-fluoro- 6-(7-fluoro-2-methyl- 2H-indazol-5- yl)imidazo[1,2- a]pyridin-2-yl)-N- methylcyclohexan-1- amine |
| 164 | 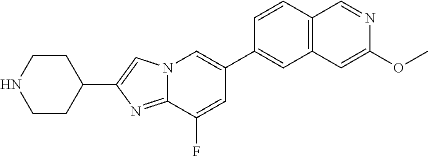 [SMILES: COc1cc2cc(-c3cc(F)c4nc(C5CCNCC5)cn4c3)ccc2cn1] | 6-(8-fluoro-2-(piperidin- 4-yl)imidazo[1,2- a]pyridin-6-yl)-3- methoxyisoquinoline |
| 165 | 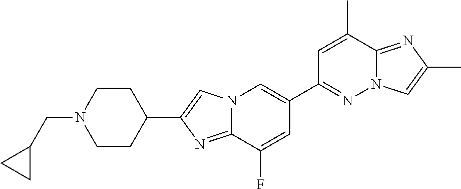 [SMILES: Cc1cn2nc(-c3cc(F)c4nc(C5CCN(CC6CC6)CC5)cn4c3)cc(C)c2n1] | 6-(2-(1- (cyclopropylmethyl) piperidin-4-yl)-8- fluoroimidazo[1,2- a]pyridin-6-yl)-2,8- dimethylimidazo[1,2- b]pyridazine |
| 166 | 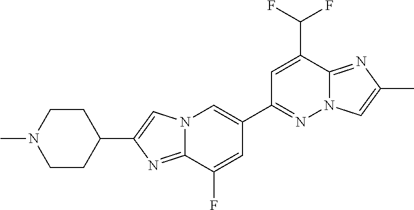 | 8-(difluoromethyl)-6-(8- fluoro-2-(1- methylpiperidin-4- yl)imidazo[1,2- a]pyridin-6-yl)-2- methylimidazo[1,2- b]pyridazine |
| 167 | 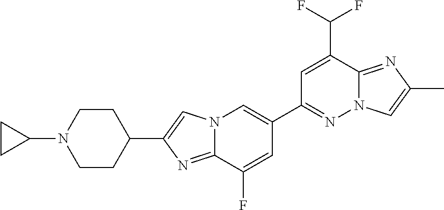 [SMILES: Cc1cn2nc(-c3cc(F)c4nc(C5CCN(C6CC6)CC5)cn4c3)cc(C(F)F)c2n1] | 6-(2-(1- cyclopropylpiperidin-4- yl)-8-fluoroimidazo[1,2- a]pyridin-6-yl)-8- (difluoromethyl)-2- methylimidazo[1,2- b]pyridazine |
| 168 | 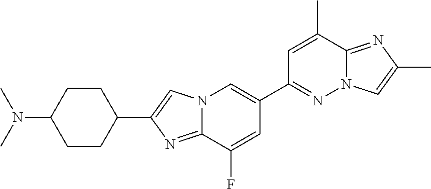 | 4-(6-(2,8- dimethylimidazo[1,2- b]pyridazin-6-yl)-8- fluoroimidazo[1,2- a]pyridin-2-yl)-N,N- dimethylcyclohexan-1- amine |
| 169 | 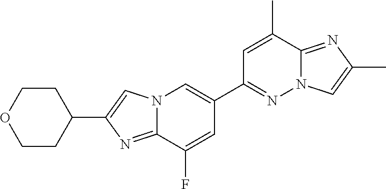 [SMILES: Cc1cn2nc(-c3cc(F)c4nc(C5CCOCC5)cn4c3)cc(C)c2n1] | 6-(8-fluoro-2- (tetrahydro-2H-pyran-4- yl)imidazo[1,2- a]pyridin-6-yl)-2,8- dimethylimidazo[1,2- b]pyridazine |
| 170 | 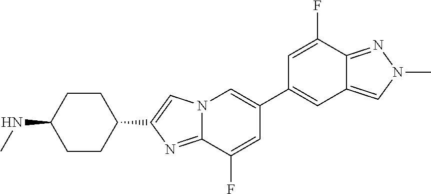 | (1r,4r)-4-(8-fluoro-6-(7- fluoro-2-methyl-2H- indazol-5- yl)imidazo[1,2- a]pyridin-2-yl)-N- methylcyclohexan-1- amine |
| 171 | 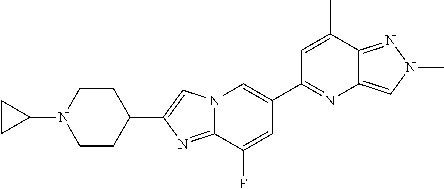 [SMILES: Cc1cc(-c2cc(F)c3nc(C4CCN(C5CC5)CC4)cn3c2)nc2cn(C)nc12] | 5-(2-(1- cyclopropylpiperidin-4- yl)-8-fluoroimidazo[1,2- a]pyridin-6-yl)-2,7- dimethyl-2H- pyrazolo[4,3-b]pyridine |
| 172 | 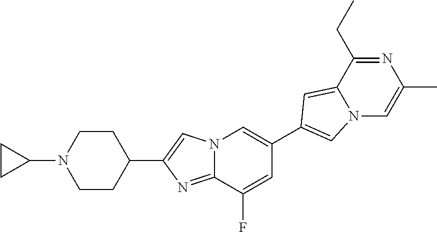 [SMILES: CCc1nc(C)cn2cc(-c3cc(F)c4nc(C5CCN(C6CC6)CC5)cn4c3)cc12] | 2-(1- cyclopropylpiperidin-4- yl)-6-(1-ethyl-3- methylpyrrolo[1,2- a]pyrazin-7-yl)-8- fluoroimidazo[1,2- a]pyridine |
| 173 | 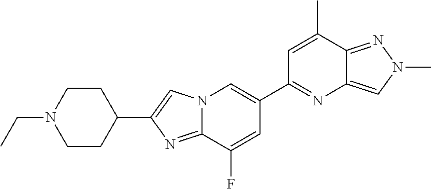 [SMILES: CCN1CCC(c2cn3cc(-c4cc(C)c5nn(C)cc5n4)cc(F)c3n2)CC1] | 5-(2-(1-ethylpiperidin-4- yl)-8-fluoroimidazo[1,2- a]pyridin-6-yl)-2,7- dimethyl-2H- pyrazolo[4,3-b]pyridine |
| 174 | 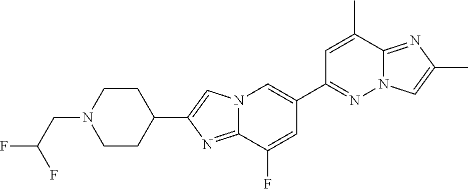 [SMILES: Cc1cn2nc(-c3cc(F)c4nc(C5CCN(CC(F)F)CC5)cn4c3)cc(C)c2n1] | 6-(2-(1-(2,2- difluoroethyl)piperidin- 4-yl)-8- fluoroimidazo[1,2- a]pyridin-6-yl)-2,8- dimethylimidazo[1,2- b]pyridazine |
| 175 | 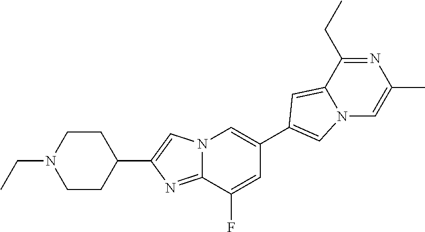 [SMILES: CCc1nc(C)cn2cc(-c3cc(F)c4nc(C5CCN(CC)CC5)cn4c3)cc12] | 6-(1-ethyl-3- methylpyrrolo[1,2- a]pyrazin-7-yl)-2-(1- ethylpiperidin-4-yl)-8- fluoroimidazo[1,2- a]pyridine |
| 176 | 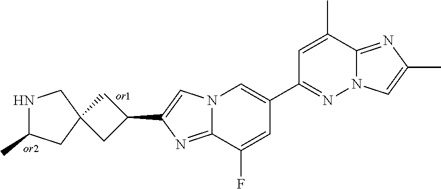 | 6-(8-fluoro-2- ((2S*,4r,7S*)-7-methyl- 6-azaspiro[3.4]octan-2- yl)imidazo[1,2- a]pyridin-6-yl)-2,8- dimethylimidazo[1,2- b]pyridazine |
| 177 | 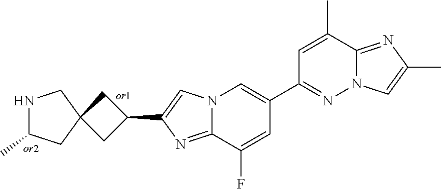 | 6-(8-fluoro-2- ((2S*,4r,7S*)-7-methyl- 6-azaspiro[3.4]octan-2- yl)imidazo[1,2- a]pyridin-6-yl)-2,8- dimethylimidazo[1,2- b]pyridazine |
| 178 | 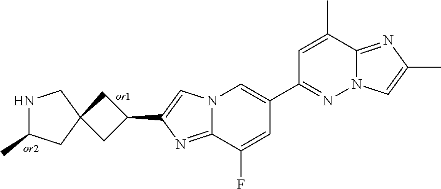 | 6-(8-fluoro-2- ((2R*,4r,7R*)-7-methyl- 6-azaspiro[3.4]octan-2- yl)imidazo[1,2- a]pyridin-6-yl)-2,8- dimethylimidazo[1,2- b]pyridazine |
| 179 | 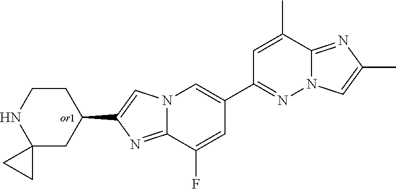 [SMILES: Cc1cn2nc(-c3cc(F)c4nc([C@@H]5CCNC6(CC6)C5)cn4c3)cc(C)c2n1] | rel-(R)-6-(8-fluoro-2-(4- azaspiro[2.5]octan-7- yl)imidazo[1,2- a]pyridin-6-yl)-2,8- dimethylimidazo[1,2- b]pyridazine |
| 180 | 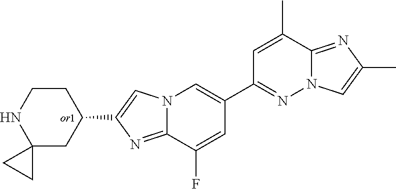 [SMILES: Cc1cn2nc(-c3cc(F)c4nc([C@H]5CCNC6(CC6)C5)cn4c3)cc(C)c2n1] | rel-(R)-6-(8-fluoro-2-(4- azaspiro[2.5]octan-7- yl)imidazo[1,2- a]pyridin-6-yl)-2,8- dimethylimidazo[1,2- b]pyridazine |
| 181 | 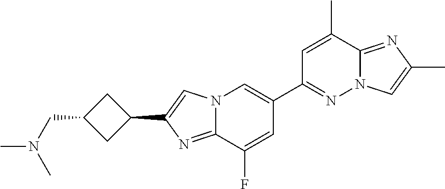 | 1-((1r,3r)-3-(6-(2,8- dimethylimidazo[1,2- b]pyridazin-6-yl)-8- fluoroimidazo[1,2- a]pyridin-2- yl)cyclobutyl)-N,N- dimethylmethanamine |
| 182 | 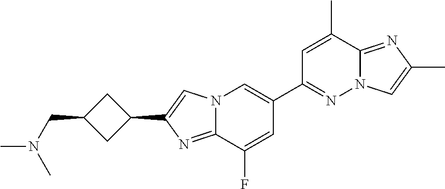 | 1-((1s,3s)-3-(6-(2,8- dimethylimidazo[1,2- b]pyridazin-6-yl)-8- fluoroimidazo[1,2- a]pyridin-2- yl)cyclobutyl)-N,N- dimethylmethanamine |
| 183 | 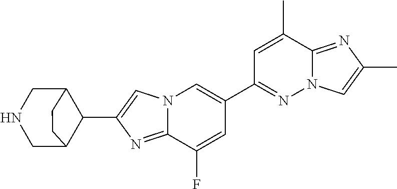 [SMILES: Cc1cn2nc(-c3cc(F)c4nc(C5C6CCC5CNC6)cn4c3)cc(C)c2n1] | 6-(2-(3- azabicyclo[3.2.1]]octan- 8-yl)-8- fluoroimidazo[1,2- a]pyridin-6-yl)-2,8- dimethylimidazo[1,2- blpyridazine |
| 184 | 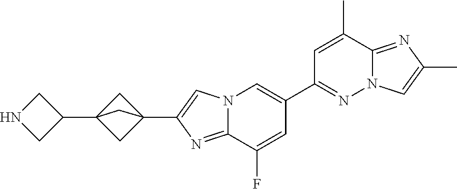 [SMILES: Cc1cn2nc(-c3cc(F)c4nc(C56CC(C7CNC7)(C5)C6)cn4c3)cc(C)c2n1] | 6-(2-(3-(azetidin-3- yl)bicyclo[1.1.1]]pentan- 1-yl)-8- fluoroimidazo[1,2- a]pyridin-6-yl)-2,8- dimethylimidazo[1,2- b]pyridazine |
| 185 | 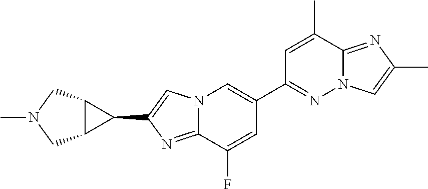 | 6-(8-fluoro-2- ((1R,5S,6r)-3-methyl-3- azabicyclo[3.1.0]hexan- 6-yl)imidazo[1,2- a]pyridin-6-yl)-2,8- dimethylimidazo[1,2- b]pyridazine |
| 186 | 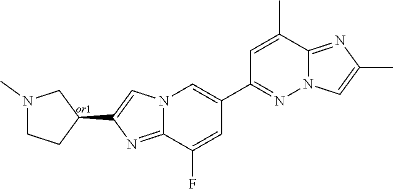 | rel-(R)-6-(8-fluoro-2-(1- methylpyrrolidin-3- yl)imidazo[1,2- a]pyridin-6-yl)-2,8- dimethylimidazo[1,2- b]pyridazine |
| 187 | 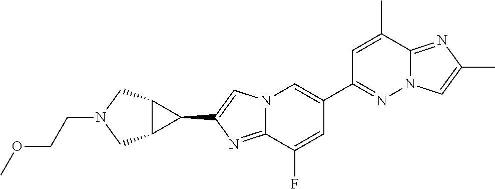 | 6-(8-fluoro-2- ((1R,5S,6r)-3-(2- methoxyethyl)-3- azabicyclo[3.1.0]hexan- 6-yl)imidazo[1,2- a]pyridin-6-yl)-2,8- dimethylimidazo[1,2- b]pyridazine |
| 188 | 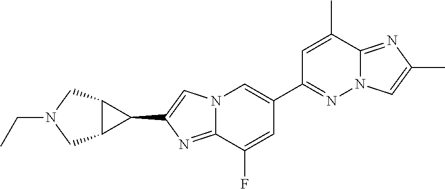 [SMILES: CCN1C[C@@H]2[C@H](C1)[C@H]2c1cn2cc(-c3cc(C)c4nc(C)cn4n3)cc(F)c2n1] | 6-(2-((1R,5S,6r)-3- ethyl-3- azabicyclo[3.1.0]hexan- 6-yl)-8- fluoroimidazo[1,2- a]pyridin-6-yl)-2,8- dimethylimidazo[1,2- b]pyridazine |
| 189 | 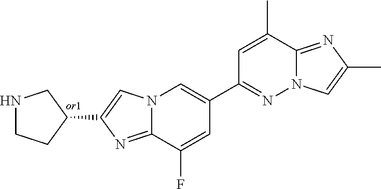 [SMILES: Cc1cn2nc(-c3cc(F)c4nc([C@@H]5CCNC5)cn4c3)cc(C)c2n1] | rel-(R)-6-(8-fluoro-2- (pyrrolidin-3- yl)imidazo[1,2- a]pyridin-6-yl)-2,8- dimethylimidazo[1,2- b]pyridazine |
| 190 | 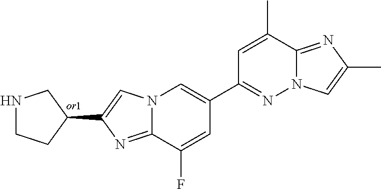 [SMILES: Cc1cn2nc(-c3cc(F)c4nc([C@H]5CCNC5)cn4c3)cc(C)c2n1] | rel-(R)-6-(8-fluoro-2- (pyrrolidin-3- yl)imidazo[1,2- a]pyridin-6-yl)-2,8- dimethylimidazo[1,2- b]pyridazine |
| 191 | 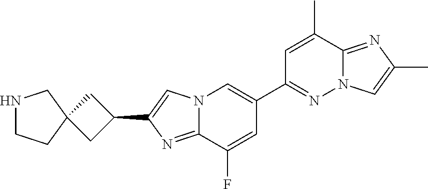 [SMILES: Cc1cn2nc(-c3cc(F)c4nc([C@H]5C[C@]6(CCNC6)C5)cn4c3)cc(C)c2n1] | 6-(8-fluoro-2-((2s,4s)-6- azaspiro[3.4]octan-2- yl)imidazo[1,2- a]pyridin-6-yl)-2,8- dimethylimidazo[1,2- b]pyridazine |
| 192 | 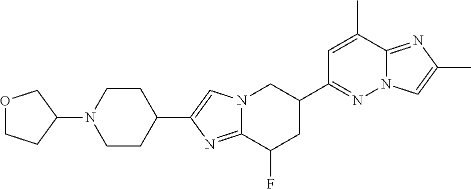 [SMILES: Cc1cn2nc(C3CC(F)c4nc(C5CCN(C6CCOC6)CC5)cn4C3)cc(C)c2n1] | 6-(8-fluoro-2-(1- (tetrahydrofuran-3- yl)piperidin-4- yl)imidazo[1,2- a]pyridin-6-yl)-2,8- dimethylimidazo[1,2- b]pyridazine |
| 193 | 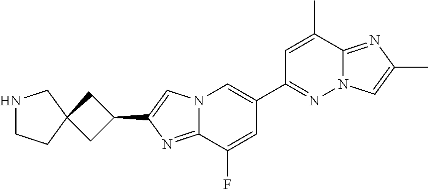 [SMILES: Cc1cn2nc(-c3cc(F)c4nc([C@H]5C[C@@]6(CCNC6)C5)cn4c3)cc(C)c2n1] | 6-(8-fluoro-2-((2r,4r)-6- azaspiro[3.4]octan-2- yl)imidazo[1,2- a]pyridin-6-yl)-2,8- dimethylimidazo[1,2- b]pyridazine |
| 194 | 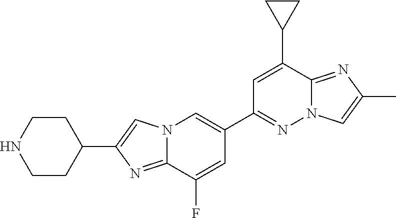 [SMILES: Cc1cn2nc(-c3cc(F)c4nc(C5CCNCC5)cn4c3)cc(C3CC3)c2n1] | 8-cyclopropyl-6-(8- fluoro-2-(piperidin-4- yl)imidazo[1,2- a]pyridin-6-yl)-2- methylimidazo[1,2- b]pyridazine |
| 195 | 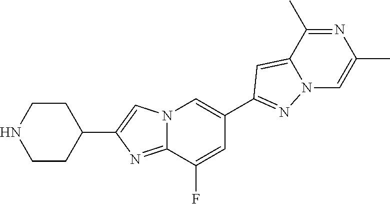 [SMILES: Cc1cn2nc(-c3cc(F)c4nc(C5CCNCC5)cn4c3)cc2c(C)n1] | 2-(8-fluoro-2-(piperidin- 4-yl)imidazo[1,2- a]pyridin-6-yl)-4,6- dimethylpyrazolo[1,5- a]pyrazine |
| 196 | 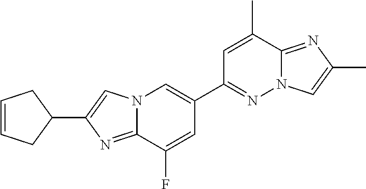 [SMILES: Cc1cn2nc(-c3cc(F)c4nc(C5CC=CC5)cn4c3)cc(C)c2n1] | 6-(2-(cyclopent-3-en-1- yl)-8-fluoroimidazo[1,2- a]pyridin-6-yl)-2,8- dimethylimidazo[1,2- b]pyridazine |
| 197 | 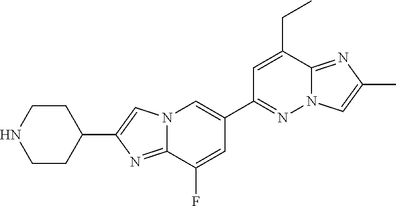 [SMILES: CCc1cc(-c2cc(F)c3nc(C4CCNCC4)cn3c2)nn2cc(C)nc12] | 8-ethyl-6-(8-fluoro-2- (piperidin-4- yl)imidazo[1,2- a]pyridin-6-yl)-2- methylimidazo[1,2- b]pyridazine |
| 198 | 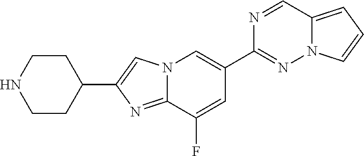 [SMILES: Fc1cc(-c2ncc3cccn3n2)cn2cc(C3CCNCC3)nc12] | 2-(8-fluoro-2-(piperidin- 4-yl)imidazo[1,2- a]pyridin-6- yl)pyrrolo[2,1- f][1,2,4]triazine |
| 199 | 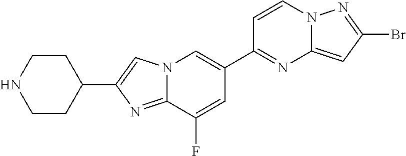 [SMILES: Fc1cc(-c2ccn3nc(Br)cc3n2)cn2cc(C3CCNCC3)nc12] | 2-bromo-5-(8-fluoro-2- (piperidin-4- yl)imidazo[1,2- a]pyridin-6- yl)pyrazolo[1,5- a]pyrimidine |
| 200 | 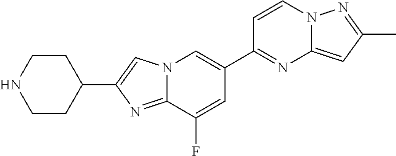 [SMILES: Cc1cc2nc(-c3cc(F)c4nc(C5CCNCC5)cn4c3)ccn2n1] | 5-(8-fluoro-2-(piperidin- 4-yl)imidazo[1,2- a]pyridin-6-yl)-2- methylpyrazolo[1,5- a]pyrimidine |
| 201 | 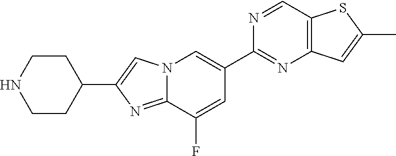 [SMILES: Cc1cc2nc(-c3cc(F)c4nc(C5CCNCC5)cn4c3)ncc2s1] | 2-(8-fluoro-2-(piperidin- 4-yl)imidazo[1,2- a]pyridin-6-yl)-6- methylthieno[3,2- d]pyrimidine |
| 202 | 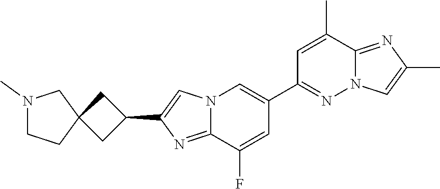 | 6-(8-fluoro-2-((2r,4r)-6- methyl-6- azaspiro[3.4]octan-2- yl)imidazo[1,2- a]pyridin-6-yl)-2,8- dimethylimidazo[1,2- b]pyridazine |
| 203 | 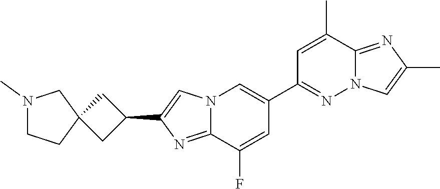 | 6-(8-fluoro-2-((2s,4s)-6- methyl-6- azaspiro[3.4]octan-2- yl)imidazo[1,2- a]pyridin-6-yl)-2,8- dimethylimidazo[1,2- b]pyridazine |
| 204 | 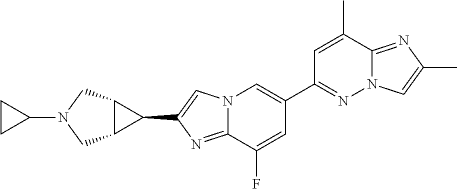 [SMILES: Cc1cn2nc(-c3cc(F)c4nc([C@H]5[C@@H]6CN(C7CC7)C[C@@H]65)cn4c3)cc(C)c2n1] | 6-(2-((1R,5S,6r)-3- cyclopropyl-3- azabicyclo[3.1.0]hexan- 6-yl)-8- fluoroimidazo[1,2- a]pyridin-6-yl)-2,8- dimethylimidazo[1,2- b]pyridazine |
| 205 | 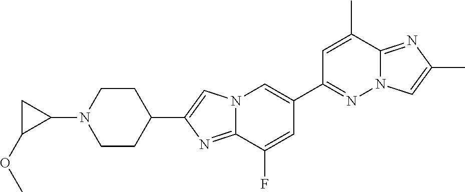 [SMILES: COC1CC1N1CCC(c2cn3cc(-c4cc(C)c5nc(C)cn5n4)cc(F)c3n2)CC1] | 6-(8-fluoro-2-(1-(2- methoxycyclopropyl) piperidin-4-yl)imidazo[1,2- a]pyridin-6-yl)-2,8- dimethylimidazo[1,2- b]pyridazine |
| 206 | 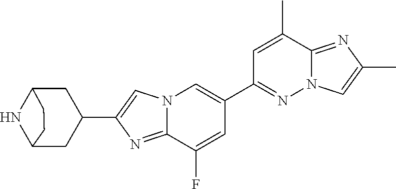 [SMILES: Cc1cn2nc(-c3cc(F)c4nc(C5CC6CCC(C5)N6)cn4c3)cc(C)c2n1] | 6-(2-(8- azabicyclo[3.2.1]]octan- 3-yl)-8- fluoroimidazo[1,2- a]pyridin-6-yl)-2,8- dimethylimidazo[1,2- b]pyridazine |
| 207 | 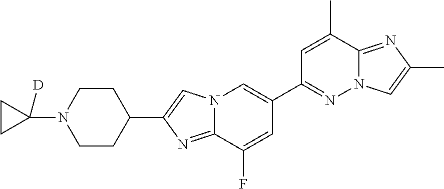 [SMILES: [2H]C1(N2CCC(c3cn4cc(-c5cc(C)c6nc(C)cn6n5)cc(F)c4n3)CC2)CC1] | 6-(2-(1-(cyclopropyl-1- d)piperidin-4-yl)-8- fluoroimidazo[1,2- a]pyridin-6-yl)-2,8- dimethylimidazo[1,2- b]pyridazine |
| 208 | 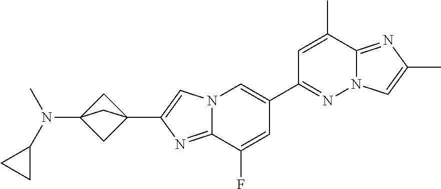 [SMILES: Cc1cn2nc(-c3cc(F)c4nc(C56CC(N(C)C7CC7)(C5)C6)cn4c3)cc(C)c2n1] | N-cyclopropyl-3-(6- (2,8- dimethylimidazo[1,2- b]pyridazin-6-yl)-8- fluoroimidazo[1,2- a]pyridin-2-yl)-N- methylbicyclo[1.1.1] pentan-1-amine |
| 209 | 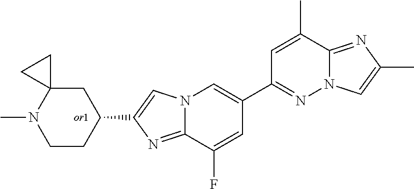 | rel-(R)-6-(8-fluoro-2-(4- methyl-4- azaspiro[2.5]octan-7- yl)imidazo[1,2- a]pyridin-6-yl)-2,8- dimethylimidazo[1,2- b]pyridazine |
| 210 | 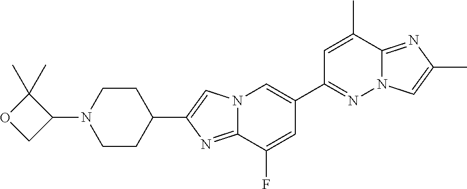 [SMILES: Cc1cn2nc(-c3cc(F)c4nc(C5CCN(C6COC6(C)C)CC5)cn4c3)cc(C)c2n1] | 6-(2-(1-(2,2- dimethyloxetan-3- yl)piperidin-4-yl)-8- fluoroimidazo[1,2- a]pyridin-6-yl)-2,8- dimethylimidazo[1,2- b]pyridazine |
| 211 | 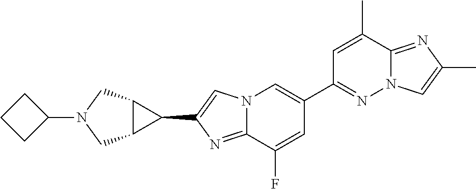 [SMILES: Cc1cn2nc(-c3cc(F)c4nc([C@H]5[C@@H]6CN(C7CCC7)C[C@@H]65)cn4c3)cc(C)c2n1] | 6-(2-((1R,5S,6r)-3- cyclobutyl-3- azabicyclo[3.1.0]hexan- 6-yl)-8- fluoroimidazo[1,2- a]pyridin-6-yl)-2,8- dimethylimidazo[1,2- b]pyridazine |
| 212 | 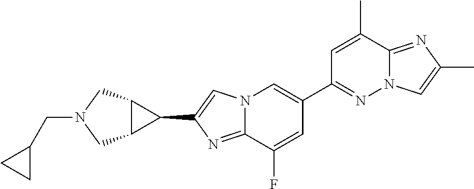 [SMILES: Cc1cn2nc(-c3cc(F)c4nc([C@H]5[C@@H]6CN(CC7CC7)C[C@@H]65)cn4c3)cc(C)c2n1] | 6-(2-((1R,5S,6r)-3- (cyclopropylmethyl)-3- azabicyclo[3.1.0]hexan- 6-yl)-8- fluoroimidazo[1,2- a]pyridin-6-yl)-2,8- dimethylimidazo[1,2- b]pyridazine |
| 213 | 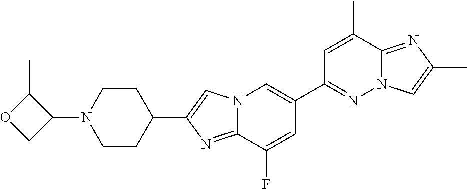 [SMILES: Cc1cn2nc(-c3cc(F)c4nc(C5CCN(C6COC6C)CC5)cn4c3)cc(C)c2n1] | 6-(8-fluoro-2-(1-(2- methyloxetan-3- yl)piperidin-4- yl)imidazo[1,2- a]pyridin-6-yl)-2,8- dimethylimidazo[1,2- b]pyridazine |
| 214 | 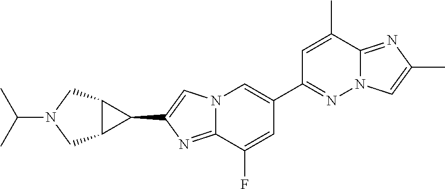 [SMILES: Cc1cn2nc(-c3cc(F)c4nc([C@H]5[C@@H]6CN(C(C)C)C[C@@H]65)cn4c3)cc(C)c2n1] | 6-(8-fluoro-2- ((1R,5S,6r)-3-isopropyl- 3- azabicyclo[3.1.0]hexan- 6-yl)imidazo[1,2- a]pyridin-6-yl)-2,8- dimethylimidazo[1,2- b]pyridazine |
| 215 | 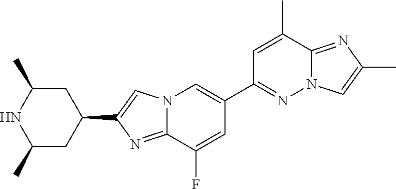 [SMILES: Cc1cn2nc(-c3cc(F)c4nc([C@H]5C[C@@H](C)N[C@@H](C)C5)cn4c3)cc(C)c2n1] | 6-(2-((2S,4r,6R)-2,6- dimethylpiperidin-4-yl)- 8-fluoroimidazo[1,2- a]pyridin-6-yl)-2,8- dimethylimidazo[1,2- b]pyridazine |
| 216 | 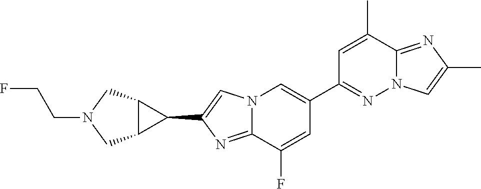 [SMILES: Cc1cn2nc(-c3cc(F)c4nc([C@H]5[C@@H]6CN(CCF)C[C@@H]65)cn4c3)cc(C)c2n1] | 6-(8-fluoro-2- ((1R,5S,6r)-3-(2- fluoroethyl)-3- azabicyclo[3.1.0]hexan- 6-yl)imidazo[1,2- a]pyridin-6-yl)-2,8- dimethylimidazo[1,2- b]pyridazine |
| 217 | 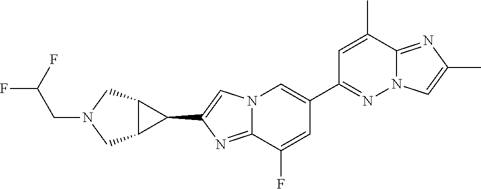 [SMILES: Cc1cn2nc(-c3cc(F)c4nc([C@H]5[C@@H]6CN(CC(F)F)C[C@@H]65)cn4c3)cc(C)c2n1] | 6-(2-((1R,5S,6r)-3-(2,2- difluoroethyl)-3- azabicyclo[3.1.0]hexan- 6-yl)-8- fluoroimidazo[1,2- a]pyridin-6-yl)-2,8- dimethylimidazo[1,2- b]pyridazine |
| 218 | 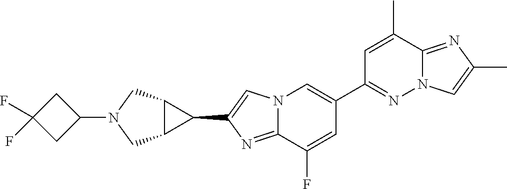 [SMILES: Cc1cn2nc(-c3cc(F)c4nc([C@H]5[C@@H]6CN(C7CC(F)(F)C7)C[C@@H]65)cn4c3)cc(C)c2n1] | 6-(2-((1R,5S,6r)-3-(3,3- difluorocyclobutyl)-3- azabicyclo[3.1.0]hexan- 6-yl)-8- fluoroimidazo[1,2- a]pyridin-6-yl)-2,8- dimethylimidazo[1,2- b]pyridazine |
| 219 | 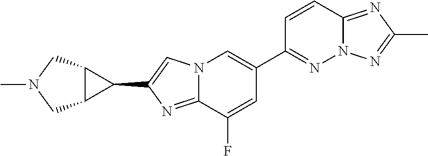 | 6-(8-fluoro-2- ((1R,5S,6r)-3-methyl-3- azabicyclo[3.1.0]hexan- 6-yl)imidazo[1,2- a]pyridin-6-yl)-2- methyl- [1,2,4]triazolo[1,5- b]pyridazine |
| 220 | 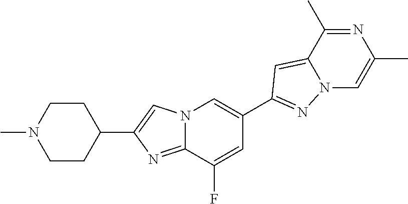 | 2-(8-fluoro-2-(1- methylpiperidin-4- yl)imidazo[1,2- a]pyridin-6-yl)-4,6- dimethylpyrazolo[1,5- a]pyrazine |
| 221 | 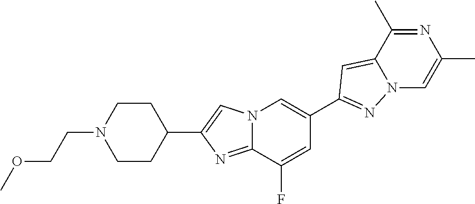 | 2-(8-fluoro-2-(1-(2- methoxyethyl)piperidin- 4-yl)imidazo[1,2- a]pyridin-6-yl)-4,6- dimethylpyrazolo[1,5- a]pyrazine |
| 222 | 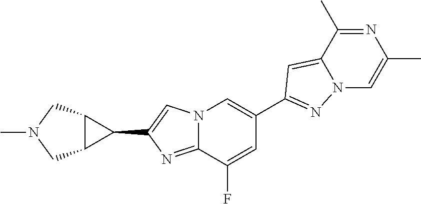 | 2-(8-fluoro-2- ((1R,5S,6r)-3-methyl-3- azabicyclo[3.1.0]hexan- 6-yl)imidazo[1,2- a]pyridin-6-yl)-4,6- dimethylpyrazolo[1,5- a]pyrazine |
| 223 | 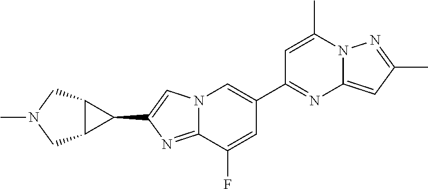 | 5-(8-fluoro-2- ((1R,5S,6r)-3-methyl-3- azabicyclo[3.1.0]hexan- 6-yl)imidazo[1,2- a]pyridin-6-yl)-2,7- dimethylpyrazolo[1,5- a]pyrimidine |
| 224 | 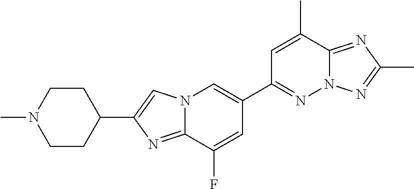 | 6-(8-fluoro-2-(1- methylpiperidin-4- yl)imidazo[1,2- a]pyridin-6-yl)-2,8- dimethyl- [1,2,4]triazolo[1,5- blpyridazine |
| 225 | 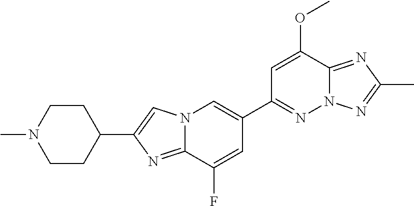 | 6-(8-fluoro-2-(1- methylpiperidin-4- yl)imidazo[1,2- a]pyridin-6-yl)-8- methoxy-2-methyl- [1,2,4]triazolo[1,5- b]pyridazine |
| 226 | 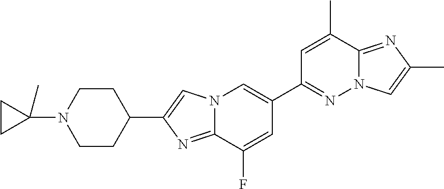 [SMILES: Cc1cn2nc(-c3cc(F)c4nc(C5CCN(C6(C)CC6)CC5)cn4c3)cc(C)c2n1] | 6-(8-fluoro-2-(1-(1- methylcyclopropyl) piperidin-4-yl)imidazo [1,2-a]pyridin-6-yl)-2,8- dimethylimidazo[1,2- b]pyridazine |
| 227 | 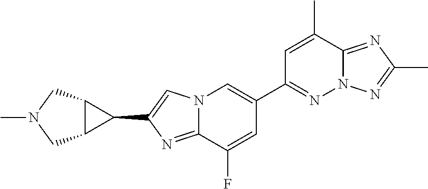 | 6-(8-fluoro-2- ((1R,5S,6r)-3-methyl-3- azabicyclo[3.1.0]hexan- 6-yl)imidazo[1,2- a]pyridin-6-yl)-2,8- dimethyl- [1,2,4]triazolo[1,5- b]pyridazine |
| 228 | 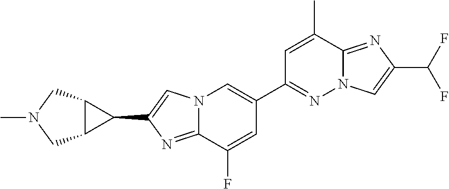 [SMILES: Cc1cc(-c2cc(F)c3nc([C@H]4[C@@H]5CN(C)C[C@@H]54)cn3c2)nn2cc(C(F)F)nc12] | 2-(difluoromethyl)-6-(8- fluoro-2-((1R,5S,6r)-3- methyl-3- azabicyclo[3.1.0]hexan- 6-yl)imidazo[1,2- a]pyridin-6-yl)-8- methylimidazo[1,2- b]pyridazine |
| 229 | 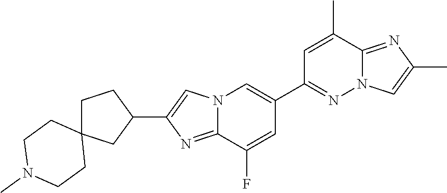 | 2-(6-(2,8- dimethylimidazo[1,2- b]pyridazin-6-yl)-8- fluoroimidazo[1,2- a]pyridin-2-yl)-8- methyl-8- azaspiro[4.5]decane |
| 230 | 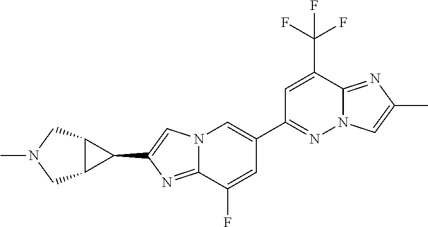 | 6-(8-fluoro-2- ((1R,5S,6r)-3-methyl-3- azabicyclo[3.1.0]hexan- 6-yl)imidazo[1,2- a]pyridin-6-yl)-2- methyl-8- (trifluoromethyl)imidazo [1,2-b]pyridazine |
| 231 | 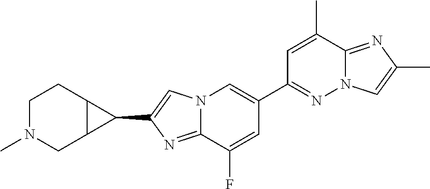 | 6-(8-fluoro-2-((7R)-3- methyl-3- azabicyclo[4.1.0]heptan- 7-yl)imidazo[1,2- a]pyridin-6-yl)-2,8- dimethylimidazo[1,2- b]pyridazine |
| 232 | 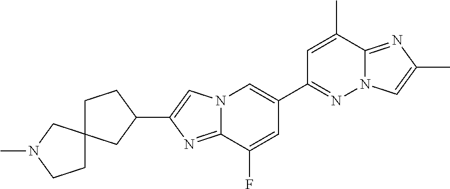 | 6-(8-fluoro-2-(2-methyl- 2-azaspiro[4.4]nonan-7- yl)imidazo[1,2- a]pyridin-6-yl)-2,8- dimethylimidazo[1,2- b]pyridazine |
| 233 | 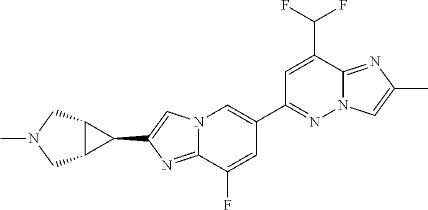 | 8-(difluoromethyl)-6-(8- fluoro-2-((1R,5S,6r)-3- methyl-3- azabicyclo[3.1.0]hexan- 6-yl)imidazo[1,2- a]pyridin-6-yl)-2- methylimidazo[1,2- b]pyridazine |
| 234 | 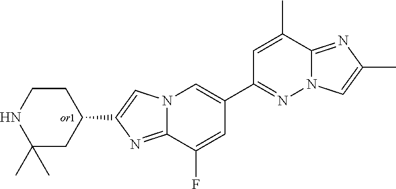 [SMILES: Cc1cn2nc(-c3cc(F)c4nc([C@H]5CCNC(C)(C)C5)cn4c3)cc(C)c2n1] | rel-(R)-6-(2-(2,2- dimethylpiperidin-4-yl)- 8-fluoroimidazo[1,2- a]pyridin-6-yl)-2,8- dimethylimidazo[1,2- b]pyridazine |
| 235 | 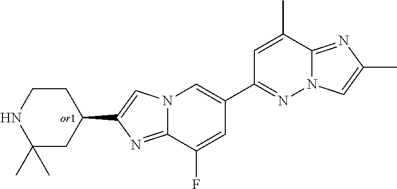 [SMILES: Cc1cn2nc(-c3cc(F)c4nc([C@@H]5CCNC(C)(C)C5)cn4c3)cc(C)c2n1] | rel-(R)-6-(2-(2,2- dimethylpiperidin-4-yl)- 8-fluoroimidazo[1,2- a]pyridin-6-yl)-2,8- dimethylimidazo[1,2- b]pyridazine |
| 236 | 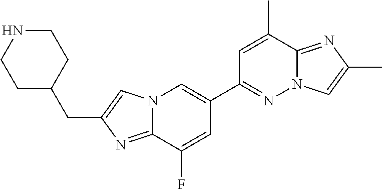 [SMILES: Cc1cn2nc(-c3cc(F)c4nc(CC5CCNCC5)cn4c3)cc(C)c2n1] | 8,8′-difluoro-2-methyl- 2′-(piperidin-4- ylmethyl)-6,6′- biimidazo[1,2- a]pyridine |
| 237 | 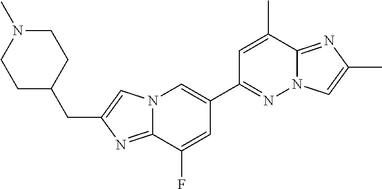 [SMILES: Cc1cn2nc(-c3cc(F)c4nc(CC5CCN(C)CC5)cn4c3)cc(C)c2n1] | 6-(8-fluoro-2-((1- methylpiperidin-4- yl)methyl)imidazo[1,2- a]pyridin-6-yl)-2,8- dimethylimidazo[1,2- b]pyridazine |
| 238 | 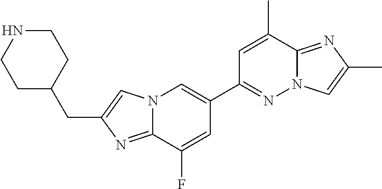 [SMILES: Cc1cn2nc(-c3cc(F)c4nc(CC5CCNCC5)cn4c3)cc(C)c2n1] | 6-(8-fluoro-2-(piperidin- 4-ylmethyl)imidazo[1,2- a]pyridin-6-yl)-2,8- dimethylimidazo[1,2- b]pyridazine |
| 239 | 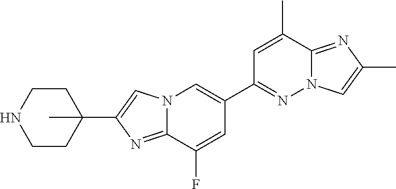 | 6-(8-fluoro-2-(4- methylpiperidin-4- yl)imidazo[1,2- a]pyridin-6-yl)-2,8- dimethylimidazo[1,2- b]pyridazine |
| 240 | 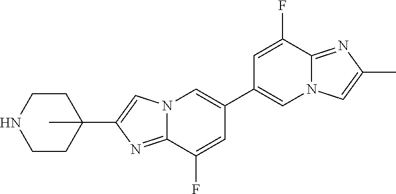 | 8,8′-difluoro-2-methyl- 2′-(4-methylpiperidin-4- yl)-6,6′-biimidazo[1,2- a]pyridine |
| 241 | 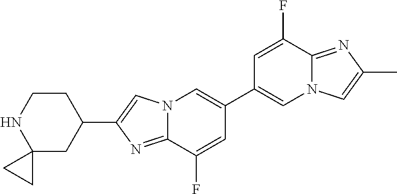 [SMILES: Cc1cn2cc(-c3cc(F)c4nc(C5CCNC6(CC6)C5)cn4c3)cc(F)c2n1] | 8,8′-difluoro-2-methyl- 2′-(4-azaspiro[2.5]octan- 7-yl)-6,6′-biimidazo[1,2- a]pyridine |
| 242 | 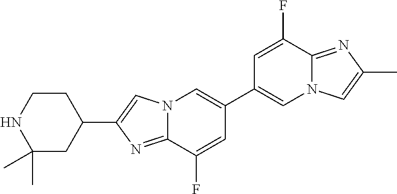 | 2-(2,2- dimethylpiperidin-4-yl)- 8,8′-difluoro-2′-methyl- 6,6′-biimidazo[1,2- a]pyridine |
| 243 | 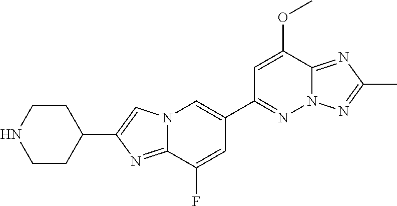 [SMILES: COc1cc(-c2cc(F)c3nc(C4CCNCC4)cn3c2)nn2nc(C)nc12] | 6-(8-fluoro-2-(piperidin- 4-yl)imidazo[1,2- a]pyridin-6-yl)-8- methoxy-2-methyl- [1,2,4]triazolo[1,5- b]pyridazine |
| 244 | 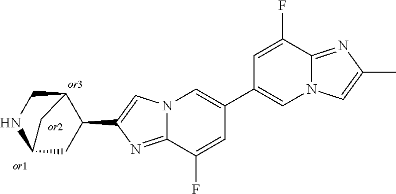 | rel-2-((1R,4R,5S)-2- azabicyclo[2.2.1]]heptan- 5-yl)-8,8′-difluoro-2′- methyl-6,6′- biimidazo[1,2- a]pyridine |
| 126 | 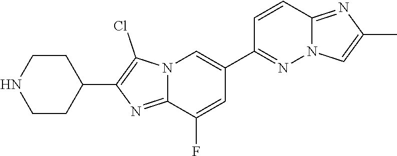 [SMILES: Cc1cn2nc(-c3cc(F)c4nc(C5CCNCC5)c(Cl)n4c3)ccc2n1] | 6-(3-chloro-8-fluoro-2- (piperidin-4- yl)imidazo[1,2- a]pyridin-6-yl)-2- methylimidazo[1,2- b]pyridazine |
| 127 | 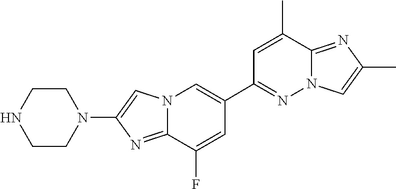 [SMILES: Cc1cn2nc(-c3cc(F)c4nc(N5CCNCC5)cn4c3)cc(C)c2n1] | 6-(8-fluoro-2- (piperazin-1- yl)imidazo[1,2- a]pyridin-6-yl)-2,8- dimethylimidazo[1,2- b]pyridazine |
| 245 | 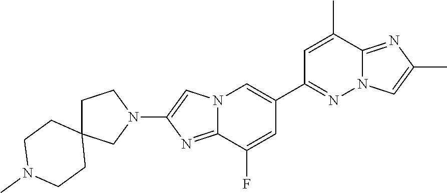 | 2-(6-(2,8- dimethylimidazo[1,2- b]pyridazin-6-yl)-8- fluoroimidazo[1,2- a]pyridin-2-yl)-8- methyl-2,8- diazaspiro[4.5]decane |
| 128 | 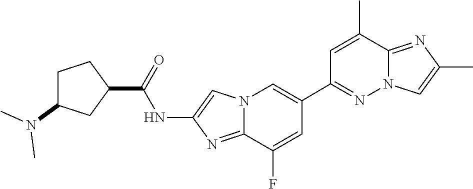 | rac-3-(dimethylamino)- N-(6-(2,8- dimethylimidazo[1,2- b]pyridazin-6-yl)-8- fluoroimidazo[1,2- a]pyridin-2- yl)cyclopentane-1- carboxamide |
| 246 | 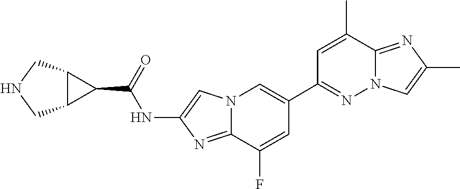 [SMILES: Cc1cn2nc(-c3cc(F)c4nc(NC(=O)[C@H]5[C@@H]6CNC[C@@H]65)cn4c3)cc(C)c2n1] | (1R,5S,6r)-N-(6-(2,8- dimethylimidazo[1,2- b]pyridazin-6-yl)-8- fluoroimidazo[1,2- a]pyridin-2-yl)-3- azabicyclo[3.1.0]hexane- 6-carboxamide |
| 247 | 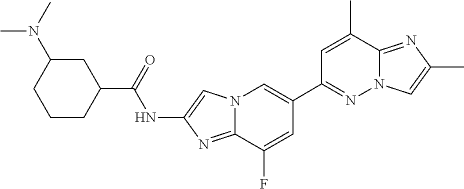 | 3-(dimethylamino)-N- (6-(2,8- dimethylimidazo[1,2- b]pyridazin-6-yl)-8- fluoroimidazo[1,2- a]pyridin-2- yl)cyclohexane-1- carboxamide |
| 248 | 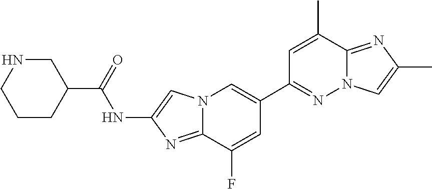 [SMILES: Cc1cn2nc(-c3cc(F)c4nc(NC(=O)C5CCCNC5)cn4c3)cc(C)c2n1] | N-(6-(2,8- dimethylimidazo[1,2- b]pyridazin-6-yl)-8- fluoroimidazo[1,2- a]pyridin-2- yl)piperidine-3- carboxamide |
| 249 | 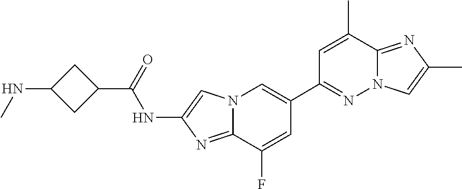 | N-(6-(2,8- dimethylimidazo[1,2- b]pyridazin-6-yl)-8- fluoroimidazo[1,2- a]pyridin-2-yl)-3- (methylamino)cyclobuta ne-1-carboxamide |
| 250 | 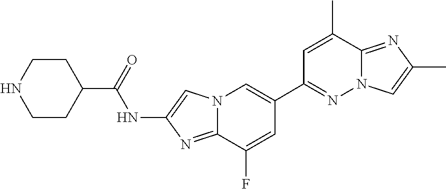 [SMILES: Cc1cn2nc(-c3cc(F)c4nc(NC(=O)C5CCNCC5)cn4c3)cc(C)c2n1] | N-(6-(2,8- dimethylimidazo[1,2- b]pyridazin-6-yl)-8- fluoroimidazo[1,2- a]pyridin-2- yl)piperidine-4- carboxamide |
| 251 | 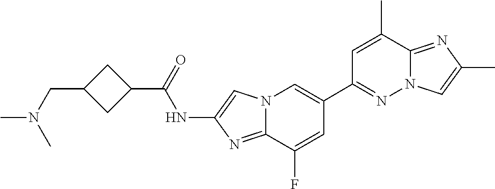 | 3- ((dimethylamino)methyl)- N-(6-(2,8- dimethylimidazo[1,2- b]pyridazin-6-yl)-8- fluoroimidazo[1,2- a]pyridin-2- yl)cyclobutane-1- carboxamide |
| 252 | 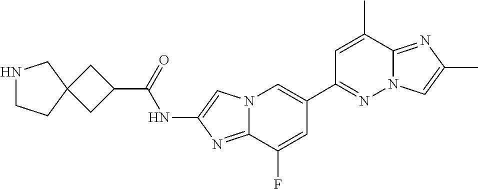 [SMILES: Cc1cn2nc(-c3cc(F)c4nc(NC(=O)C5CC6(CCNC6)C5)cn4c3)cc(C)c2n1] | N-(6-(2,8- dimethylimidazo[1,2- b]pyridazin-6-yl)-8- fluoroimidazo[1,2- a]pyridin-2-yl)-6- azaspiro[3.4]octane-2- carboxamide |
| 253 | 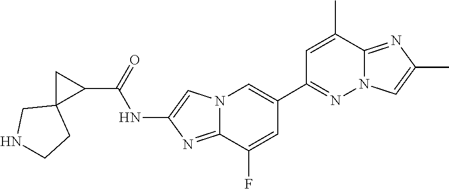 [SMILES: Cc1cn2nc(-c3cc(F)c4nc(NC(=O)C5CC56CCNC6)cn4c3)cc(C)c2n1] | N-(6-(2,8- dimethylimidazo[1,2- b]pyridazin-6-yl)-8- fluoroimidazo[1,2- a]pyridin-2-yl)-5- azaspiro[2.4]heptane-1- carboxamide |
| 254 | 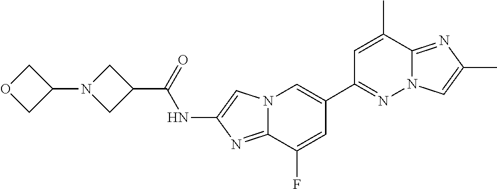 [SMILES: Cc1cn2nc(-c3cc(F)c4nc(NC(=O)C5CN(C6COC6)C5)cn4c3)cc(C)c2n1] | N-(6-(2,8- dimethylimidazo[1,2- b]pyridazin-6-yl)-8- fluoroimidazo[1,2- a]pyridin-2-yl)-1- (oxetan-3-yl)azetidine- 3-carboxamide |
| 255 | 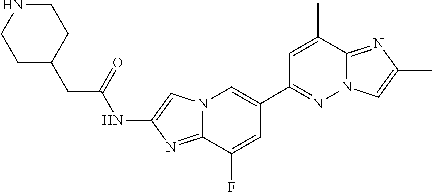 [SMILES: Cc1cn2nc(-c3cc(F)c4nc(NC(=O)CC5CCNCC5)cn4c3)cc(C)c2n1] | N-(6-(2,8- dimethylimidazo[1,2- b]pyridazin-6-yl)-8- fluoroimidazo[1,2- a]pyridin-2-yl)-2- (piperidin-3- yl)acetamide |
| 256 | 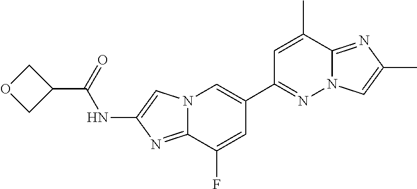 [SMILES: Cc1cn2nc(-c3cc(F)c4nc(NC(=O)C5COC5)cn4c3)cc(C)c2n1] | N-(6-(2,8- dimethylimidazo[1,2- b]pyridazin-6-yl)-8- fluoroimidazo[1,2- a]pyridin-2-yl)oxetane- 3-carboxamide |
| 257 | 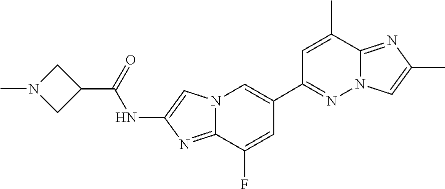 | N-(6-(2,8- dimethylimidazo[1,2- b]pyridazin-6-yl)-8- fluoroimidazo[1,2- a]pyridin-2-yl)-1- methylazetidine-3- carboxamide |
| 258 | 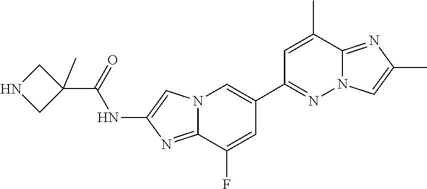 [SMILES: Cc1cn2nc(-c3cc(F)c4nc(NC(=O)C5(C)CNC5)cn4c3)cc(C)c2n1] | N-(6-(2,8- dimethylimidazo[1,2- b]pyridazin-6-yl)-8- fluoroimidazo[1,2- a]pyridin-2-yl)-3- methylazetidine-3- carboxamide |
| 259 | 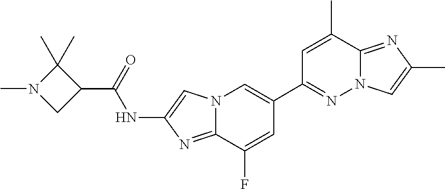 | N-(6-(2,8- dimethylimidazo[1,2- b]pyridazin-6-yl)-8- fluoroimidazo[1,2- a]pyridin-2-yl)-1,2,2- trimethylazetidine-3- carboxamide |
| 260 | 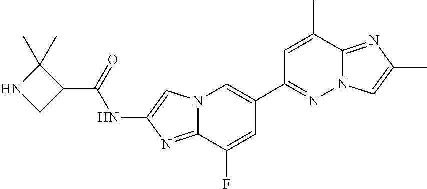 [SMILES: Cc1cn2nc(-c3cc(F)c4nc(NC(=O)C5CNC5(C)C)cn4c3)cc(C)c2n1] | N-(6-(2,8- dimethylimidazo[1,2- b]pyridazin-6-yl)-8- fluoroimidazo[1,2- a]pyridin-2-yl)-2,2- dimethylazetidine-3- carboxamide |
| 261 | 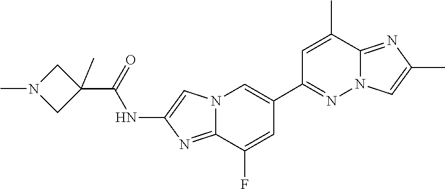 | N-(6-(2,8- dimethylimidazo[1,2- b]pyridazin-6-yl)-8- fluoroimidazo[1,2- a]pyridin-2-yl)-1,3- dimethylazetidine-3- carboxamide |
| 262 | 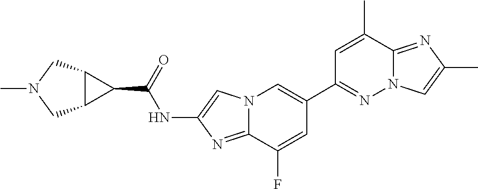 | (1R,5S,6r)-N-(6-(2,8- dimethylimidazo[1,2- b]pyridazin-6-yl)-8- fluoroimidazo[1,2- a]pyridin-2-yl)-3- methyl-3- azabicyclo[3.1.0]hexane- 6-carboxamide |
| 263 | 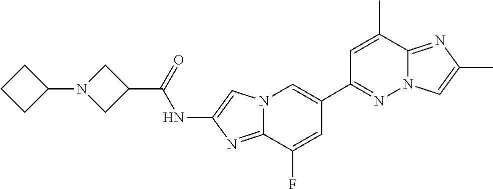 [SMILES: Cc1cn2nc(-c3cc(F)c4nc(NC(=O)C5CN(C6CCC6)C5)cn4c3)cc(C)c2n1] | 1-cyclobutyl-N-(6-(2,8- dimethylimidazo[1,2- b]pyridazin-6-yl)-8- fluoroimidazo[1,2- a]pyridin-2-yl)azetidine- 3-carboxamide |
| 264 | 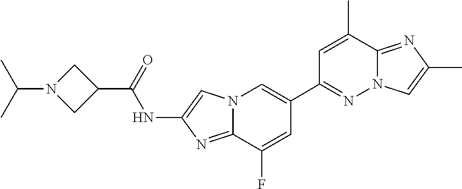 [SMILES: Cc1cn2nc(-c3cc(F)c4nc(NC(=O)C5CN(C(C)C)C5)cn4c3)cc(C)c2n1] | N-(6-(2,8- dimethylimidazo[1,2- b]pyridazin-6-yl)-8- fluoroimidazo[1,2- a]pyridin-2-yl)-1- isopropylazetidine-3- carboxamide |
| 265 | 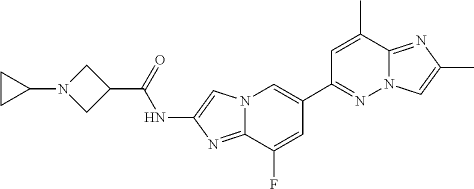 [SMILES: Cc1cn2nc(-c3cc(F)c4nc(NC(=O)C5CN(C6CC6)C5)cn4c3)cc(C)c2n1] | 1-cyclopropyl-N-(6- (2,8- dimethylimidazo[1,2- b]pyridazin-6-yl)-8- fluoroimidazo[1,2- a]pyridin-2-yl)azetidine- 3-carboxamide |
| 266 | 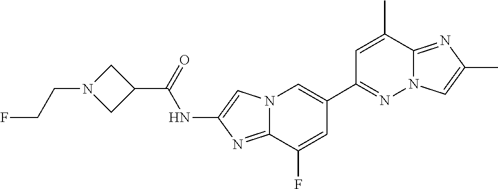 [SMILES: Cc1cn2nc(-c3cc(F)c4nc(NC(=O)C5CN(CCF)C5)cn4c3)cc(C)c2n1] | N-(6-(2,8- dimethylimidazo[1,2- b]pyridazin-6-yl)-8- fluoroimidazo[1,2- a]pyridin-2-yl)-1-(2- fluoroethyl)azetidine-3- carboxamide |
| 267 | 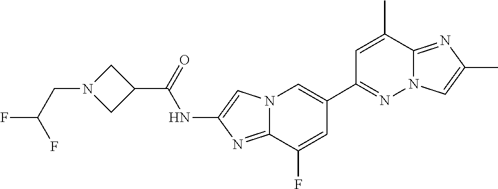 [SMILES: Cc1cn2nc(-c3cc(F)c4nc(NC(=O)C5CN(CC(F)F)C5)cn4c3)cc(C)c2n1] | 1-(2,2-difluoroethyl)-N- (6-(2,8- dimethylimidazo[1,2- b]pyridazin-6-yl)-8- fluoroimidazo[1,2- a]pyridin-2-yl)azetidine- 3-carboxamide |
| 268 | 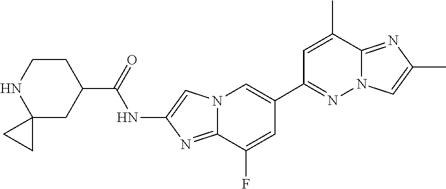 [SMILES: Cc1cn2nc(-c3cc(F)c4nc(NC(=O)C5CCNC6(CC6)C5)cn4c3)cc(C)c2n1] | N-(6-(2,8- dimethylimidazo[1,2- b]pyridazin-6-yl)-8- fluoroimidazo[1,2- a]pyridin-2-yl)-4- azaspiro[2.5]octane-7- carboxamide |
| 269 | 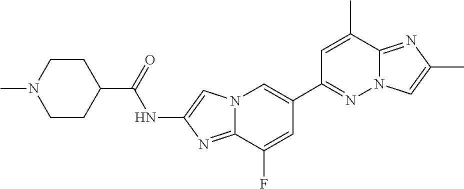 | N-(6-(2,8- dimethylimidazo[1,2- b]pyridazin-6-yl)-8- fluoroimidazo[1,2- a]pyridin-2-yl)-1- methylpiperidine-4- carboxamide |
| 270 | 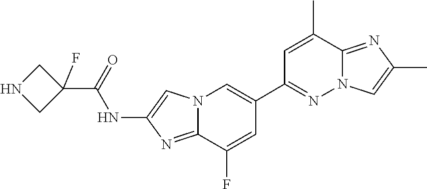 [SMILES: Cc1cn2nc(-c3cc(F)c4nc(NC(=O)C5(F)CNC5)cn4c3)cc(C)c2n1] | N-(6-(2,8- dimethylimidazo[1,2- b]pyridazin-6-yl)-8- fluoroimidazo[1,2- a]pyridin-2-yl)-3- fluoroazetidine-3- carboxamide |
| 271 | 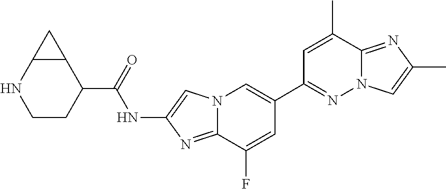 [SMILES: Cc1cn2nc(-c3cc(F)c4nc(NC(=O)C5CCNC6CC65)cn4c3)cc(C)c2n1] | N-(6-(2,8- dimethylimidazo[1,2- b]pyridazin-6-yl)-8- fluoroimidazo[1,2- a]pyridin-2-yl)-2- azabicyclo[4.1.0] heptane-5-carboxamide |
| 272 | 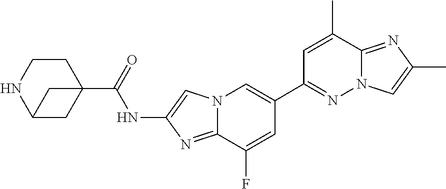 [SMILES: Cc1cn2nc(-c3cc(F)c4nc(NC(=O)C56CCNC(C5)C6)cn4c3)cc(C)c2n1] | N-(6-(2,8- dimethylimidazo[1,2- b]pyridazin-6-yl)-8- fluoroimidazo[1,2- a]pyridin-2-yl)-3- azabicyclo[3.1.1] heptane-1-carboxamide |
| 273 | 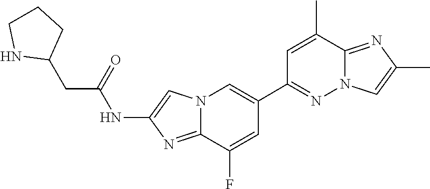 [SMILES: Cc1cn2nc(-c3cc(F)c4nc(NC(=O)CC5CCCN5)cn4c3)cc(C)c2n1] | N-(6-(2,8- dimethylimidazo[1,2- b]pyridazin-6-yl)-8- fluoroimidazo[1,2- a]pyridin-2-yl)-2- (pyrrolidin-2- yl)acetamide |
| 274 | 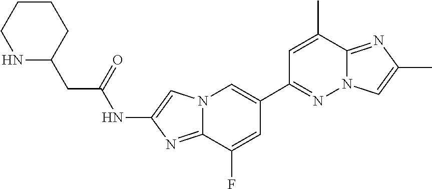 [SMILES: Cc1cn2nc(-c3cc(F)c4nc(NC(=O)CC5CCCCN5)cn4c3)cc(C)c2n1] | N-(6-(2,8- dimethylimidazo[1,2- b]pyridazin-6-yl)-8- fluoroimidazo[1,2- a]pyridin-2-yl)-2- (piperidin-2- yl)acetamide |
| 275 | 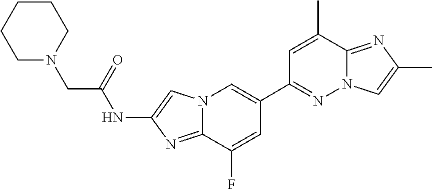 [SMILES: Cc1cn2nc(-c3cc(F)c4nc(NC(=O)CN5CCCCC5)cn4c3)cc(C)c2n1] | N-(6-(2,8- dimethylimidazo[1,2- b]pyridazin-6-yl)-8- fluoroimidazo[1,2- a]pyridin-2-yl)-2- (piperidin-1- yl)acetamide |
| 276 | 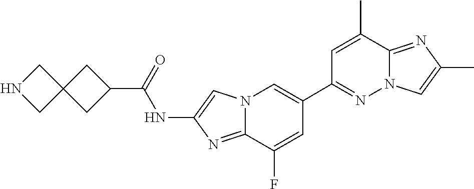 [SMILES: Cc1cn2nc(-c3cc(F)c4nc(NC(=O)C5CC6(CNC6)C5)cn4c3)cc(C)c2n1] | N-(6-(2,8- dimethylimidazo[1,2- b]pyridazin-6-yl)-8- fluoroimidazo[1,2- a]pyridin-2-yl)-2- azaspiro[3.3]]heptane-6- carboxamide |
| 277 | 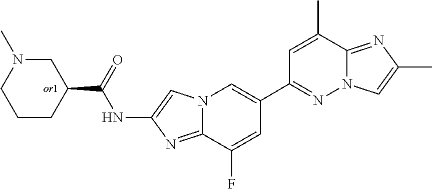 [SMILES: Cc1cn2nc(-c3cc(F)c4nc(NC(=O)[C@H]5CCCN(C)C5)cn4c3)cc(C)c2n1] | rel-(R)-N-(6-(2,8- dimethylimidazo[1,2- b]pyridazin-6-yl)-8- fluoroimidazo[1,2- a]pyridin-2-yl)-1- methylpiperidine-3- carboxamide |
| 278 | 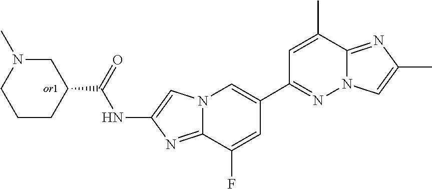 [SMILES: Cc1cn2nc(-c3cc(F)c4nc(NC(=O)[C@@H]5CCCN(C)C5)cn4c3)cc(C)c2n1] | rel-(R)-N-(6-(2,8- dimethylimidazo[1,2- b]pyridazin-6-yl)-8- fluoroimidazo[1,2- a]pyridin-2-yl)-1- methylpiperidine-3- carboxamide |
| 279 | 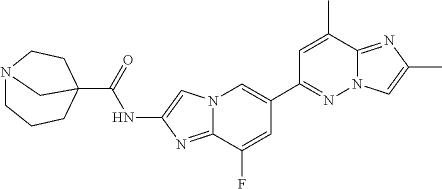 [SMILES: Cc1cn2nc(-c3cc(F)c4nc(NC(=O)C56CCCN(CC5)C6)cn4c3)cc(C)c2n1] | N-(6-(2,8- dimethylimidazo[1,2- b]pyridazin-6-yl)-8- fluoroimidazo[1,2- a]pyridin-2-yl)-1- azabicyclo[3.2.1]]octane- 5-carboxamide |
| 280 | 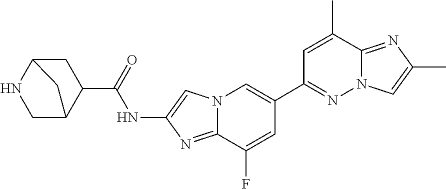 [SMILES: Cc1cn2nc(-c3cc(F)c4nc(NC(=O)C5CC6CC5CN6)cn4c3)cc(C)c2n1] | N-(6-(2,8- dimethylimidazo[1,2- b]pyridazin-6-yl)-8- fluoroimidazo[1,2- a]pyridin-2-yl)-2- azabicyclo[2.2.1]]heptane- 5-carboxamide |
| 281 | 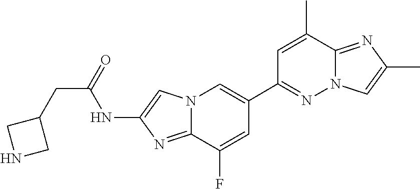 [SMILES: Cc1cn2nc(-c3cc(F)c4nc(NC(=O)CC5CNC5)cn4c3)cc(C)c2n1] | 2-(azetidin-3-yl)-N-(6- (2,8- dimethylimidazo[1,2- b]pyridazin-6-yl)-8- fluoroimidazo[1,2- a]pyridin-2- yl)acetamide |
| 282 | 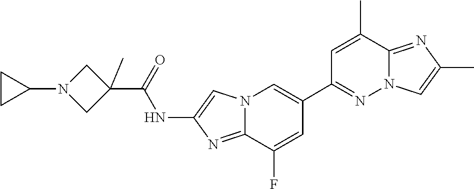 [SMILES: Cc1cn2nc(-c3cc(F)c4nc(NC(=O)C5(C)CN(C6CC6)C5)cn4c3)cc(C)c2n1] | 1-cyclopropyl-N-(6- (2,8- dimethylimidazo[1,2- b]pyridazin-6-yl)-8- fluoroimidazo[1,2- a]pyridin-2-yl)-3- methylazetidine-3- carboxamide |
| 283 | 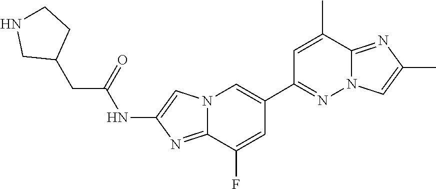 [SMILES: Cc1cn2nc(-c3cc(F)c4nc(NC(=O)CC5CCNC5)cn4c3)cc(C)c2n1] | N-(6-(2,8- dimethylimidazo[1,2- b]pyridazin-6-yl)-8- fluoroimidazo[1,2- a]pyridin-2-yl)-2- (pyrrolidin-3- yl)acetamide |
| 284 | 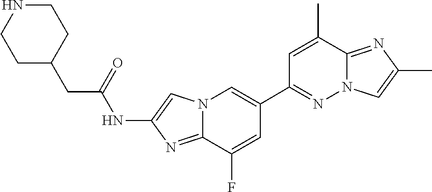 [SMILES: Cc1cn2nc(-c3cc(F)c4nc(NC(=O)CC5CCNCC5)cn4c3)cc(C)c2n1] | N-(6-(2,8- dimethylimidazo[1,2- b]pyridazin-6-yl)-8- fluoroimidazo[1,2- a]pyridin-2-yl)-2- (piperidin-4- yl)acetamide |
| 285 | 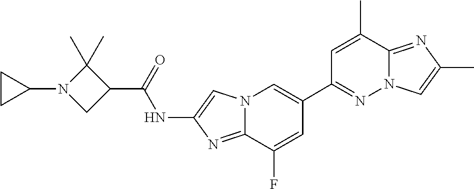 [SMILES: Cc1cn2nc(-c3cc(F)c4nc(NC(=O)C5CN(C6CC6)C5(C)C)cn4c3)cc(C)c2n1] | 1-cyclopropyl-N-(6- (2,8- dimethylimidazo[1,2- b]pyridazin-6-yl)-8- fluoroimidazo[1,2- a]pyridin-2-yl)-2,2- dimethylazetidine-3- carboxamide |
| 286 | 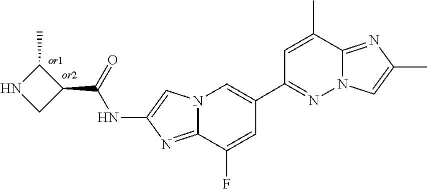 [SMILES: Cc1cn2nc(-c3cc(F)c4nc(NC(=O)[C@H]5CN[C@@H]5C)cn4c3)cc(C)c2n1] | rel-(2R,3S)-N-(6-(2,8- dimethylimidazo[1,2- b]pyridazin-6-yl)-8- fluoroimidazo[1,2- a]pyridin-2-yl)-2- methylazetidine-3- carboxamide |
| 287 | 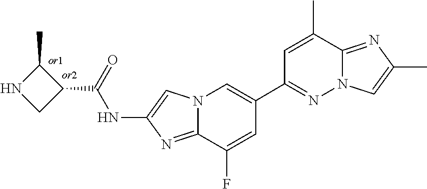 [SMILES: Cc1cn2nc(-c3cc(F)c4nc(NC(=O)[C@@H]5CN[C@H]5C)cn4c3)cc(C)c2n1] | rel-(2R,3S)-N-(6-(2,8- dimethylimidazo[1,2- b]pyridazin-6-yl)-8- fluoroimidazo[1,2- a]pyridin-2-yl)-2- methylazetidine-3- carboxamide |
| 288 | 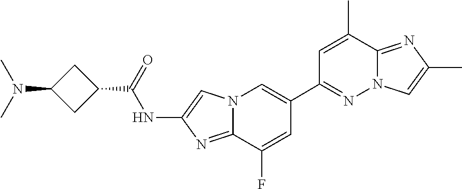 | (1r,3r)-3- (dimethylamino)-N-(6- (2,8- dimethylimidazo[1,2- b]pyridazin-6-yl)-8- fluoroimidazo[1,2- a]pyridin-2- yl)cyclobutane-1- carboxamide |
| 289 | 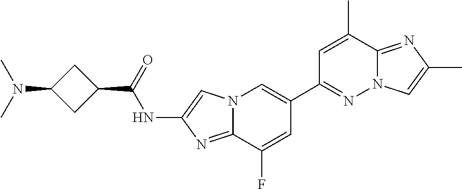 | (1s,3s)-3- (dimethylamino)-N-(6- (2,8- dimethylimidazo[1,2- b]pyridazin-6-yl)-8- fluoroimidazo[1,2- a]pyridin-2- yl)cyclobutane-1- carboxamide |
| 290 | 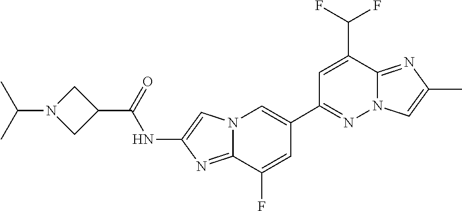 [SMILES: Cc1cn2nc(-c3cc(F)c4nc(NC(=O)C5CN(C(C)C)C5)cn4c3)cc(C(F)F)c2n1] | N-(6-(8- (difluoromethyl)-2- methylimidazo[1,2- b]pyridazin-6-yl)-8- fluoroimidazo[1,2- a]pyridin-2-yl)-1- isopropylazetidine-3- carboxamide |
| 291 | 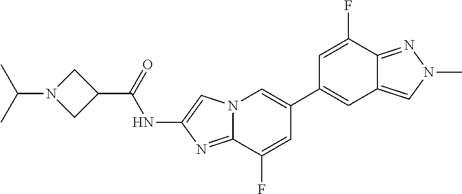 [SMILES: CC(C)N1CC(C(=O)Nc2cn3cc(-c4cc(F)c5nn(C)cc5c4)cc(F)c3n2)C1] | N-(8-fluoro-6-(7-fluoro- 2-methyl-2H-indazol-5- yl)imidazo[1,2- a]pyridin-2-yl)-1- isopropylazetidine-3- carboxamide |
| 292 | 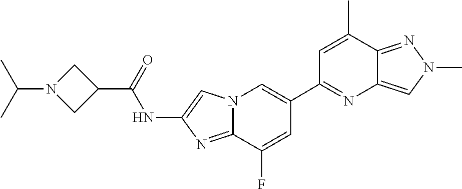 [SMILES: Cc1cc(-c2cc(F)c3nc(NC(=O)C4CN(C(C)C)C4)cn3c2)nc2cn(C)nc12] | N-(6-(2,7-dimethyl-2H- pyrazolo[4,3-b]pyridin- 5-yl)-8- fluoroimidazo[1,2- a]pyridin-2-yl)-1- isopropylazetidine-3- carboxamide |
| 293 | 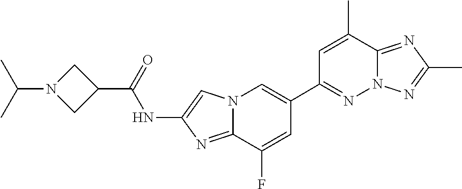 [SMILES: Cc1nc2c(C)cc(-c3cc(F)c4nc(NC(=O)C5CN(C(C)C)C5)cn4c3)nn2n1] | N-(6-(2,8-dimethyl- [1,2,4]triazolo[1,5- b]pyridazin-6-yl)-8- fluoroimidazo[1,2- a]pyridin-2-yl)-1- isopropylazetidine-3- carboxamide |
| 294 | 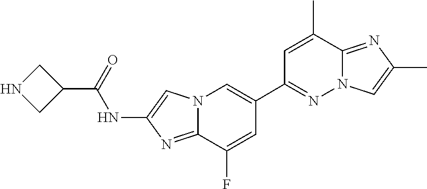 [SMILES: Cc1cn2nc(-c3cc(F)c4nc(NC(=O)C5CNC5)cn4c3)cc(C)c2n1] | N-(6-(2,8- dimethylimidazo[1,2- b]pyridazin-6-yl)-8- fluoroimidazo[1,2- a]pyridin-2-yl)azetidine- 3-carboxamide |
| 295 | 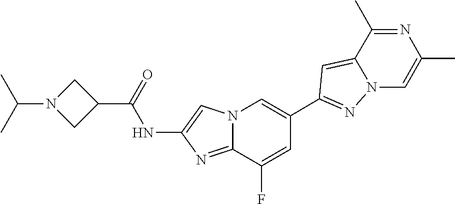 [SMILES: Cc1cn2nc(-c3cc(F)c4nc(NC(=O)C5CN(C(C)C)C5)cn4c3)cc2c(C)n1] | N-(6-(4,6- dimethylpyrazolo[1,5- a]pyrazin-2-yl)-8- fluoroimidazo[1,2- a]pyridin-2-yl)-1- isopropylazetidine-3- carboxamide |
| 296 | 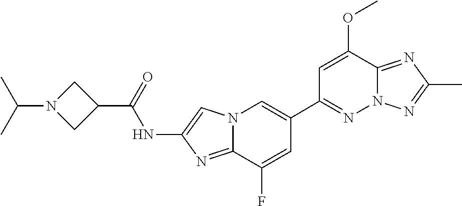 [SMILES: COc1cc(-c2cc(F)c3nc(NC(=O)C4CN(C(C)C)C4)cn3c2)nn2nc(C)nc12] | N-(8-fluoro-6-(8- methoxy-2-methyl- [1,2,4]triazolo[1,5- b]pyridazin-6- yl)imidazo[1,2- a]pyridin-2-yl)-1- isopropylazetidine-3- carboxamide |
| 297 | 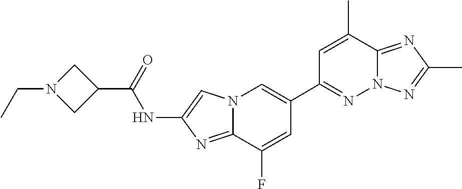 [SMILES: CCN1CC(C(=O)Nc2cn3cc(-c4cc(C)c5nc(C)nn5n4)cc(F)c3n2)C1] | N-(6-(2,8-dimethyl- [1,2,4]triazolo[1,5- b]pyridazin-6-yl)-8- fluoroimidazo[1,2- a]pyridin-2-yl)-1- ethylazetidine-3- carboxamide |
| 298 | 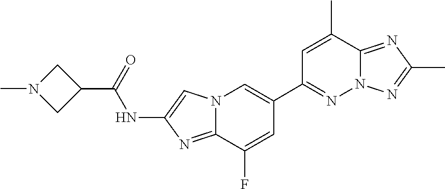 | N-(6-(2,8-dimethyl- [1,2,4]triazolo[1,5- b]pyridazin-6-yl)-8- fluoroimidazo[1,2- a]pyridin-2-yl)-1- methylazetidine-3- carboxamide |
| 299 | 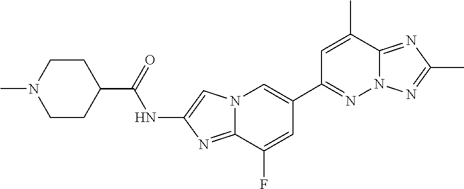 | N-(6-(2,8-dimethyl- [1,2,4]triazolo[1,5- b]pyridazin-6-yl)-8- fluoroimidazo[1,2- a]pyridin-2-yl)-1- methylpiperidine-4- carboxamide |
| 300 | 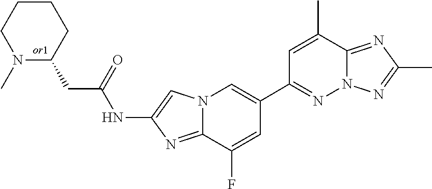 [SMILES: Cc1nc2c(C)cc(-c3cc(F)c4nc(NC(=O)C[C@H]5CCCCN5C)cn4c3)nn2n1] | rel-(R)-N-(6-(2,8- dimethylimidazo[1,2- b]pyridazin-6-yl)-8- fluoroimidazo[1,2- a]pyridin-2-yl)-2-(1- methylpiperidin-2- yl)acetamide |
| 301 | 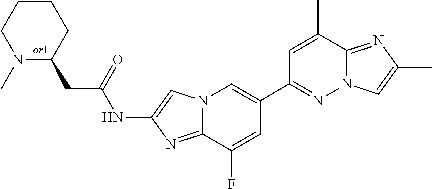 [SMILES: Cc1cn2nc(-c3cc(F)c4nc(NC(=O)C[C@@H]5CCCCN5C)cn4c3)cc(C)c2n1] | rel-(R)-N-(6-(2,8- dimethylimidazo[1,2- b]pyridazin-6-yl)-8- fluoroimidazo[1,2- a]pyridin-2-yl)-2-(1- methylpiperidin-2- yl)acetamide |
| 302 | 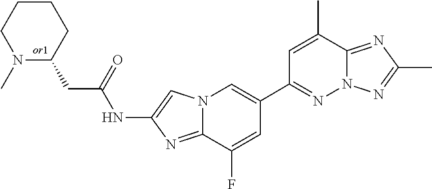 [SMILES: Cc1nc2c(C)cc(-c3cc(F)c4nc(NC(=O)C[C@H]5CCCCN5C)cn4c3)nn2n1] | rel-(R)-N-(6-(2,8- dimethyl- [1,2,4]triazolo[1,5- b]pyridazin-6-yl)-8- fluoroimidazo[1,2- a]pyridin-2-yl)-2-(1- methylpiperidin-2- yl)acetamide |
| 303 | 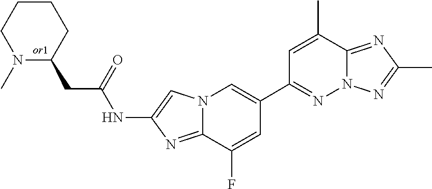 [SMILES: Cc1nc2c(C)cc(-c3cc(F)c4nc(NC(=O)C[C@@H]5CCCCN5C)cn4c3)nn2n1] | rel-(R)-N-(6-(2,8- dimethyl- [1,2,4]triazolo[1,5- b]pyridazin-6-yl)-8- fluoroimidazo[1,2- a]pyridin-2-yl)-2-(1- methylpiperidin-2- yl)acetamide |
| 304 | 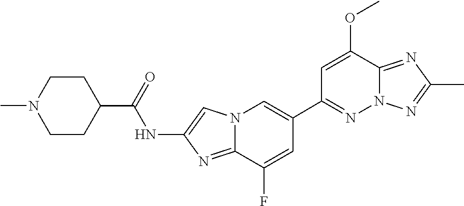 | N-(8-fluoro-6-(8- methoxy-2-methyl- [1,2,4]triazolo[1,5- b]pyridazin-6- yl)imidazo[1,2- a]pyridin-2-yl)-1- methylpiperidine-4- carboxamide |
| 305 | 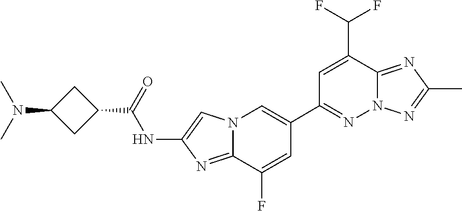 | (1r,3r)-N-(6-(8- (difluoromethyl)-2- methyl- [1,2,4]triazolo[1,5- b]pyridazin-6-yl)-8- fluoroimidazo[1,2- a]pyridin-2-yl)-3- (dimethylamino) cyclobutane-1- carboxamide |
| 306 | 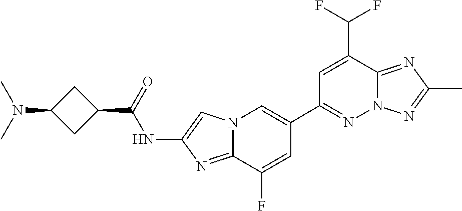 | (1s,3s)-N-(6-(8- (difluoromethyl)-2- methyl- [1,2,4]triazolo[1,5- b]pyridazin-6-yl)-8- fluoroimidazo[1,2- a]pyridin-2-yl)-3- (dimethylamino) cyclobutane-1- carboxamide |
| 307 | 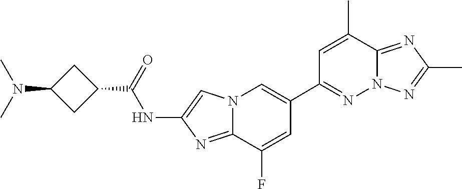 | (1r,3r)-N-(6-(2,8- dimethyl- [1,2,4]triazolo[1,5- b]pyridazin-6-yl)-8- fluoroimidazo[1,2- a]pyridin-2-yl)-3- (dimethylamino) cyclobutane-1- carboxamide |
| 308 | 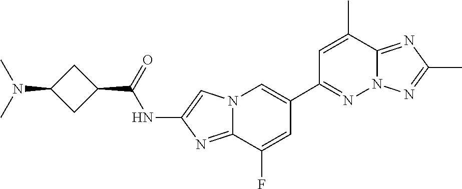 | (1s,3s)-N-(6-(2,8- dimethyl- [1,2,4]triazolo[1,5- b]pyridazin-6-yl)-8- fluoroimidazo[1,2- a]pyridin-2-yl)-3- (dimethylamino) cyclobutane-1- carboxamide |
| 309 | 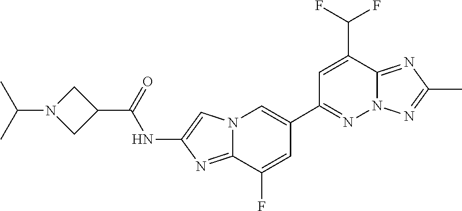 [SMILES: Cc1nc2c(C(F)F)cc(-c3cc(F)c4nc(NC(=O)C5CN(C(C)C)C5)cn4c3)nn2n1] | N-(6-(8- (difluoromethyl)-2- methyl- [1,2,4]triazolo[1,5- b]pyridazin-6-yl)-8- fluoroimidazo[1,2- a]pyridin-2-yl)-1- isopropylazetidine-3- carboxamide |
| 310 | 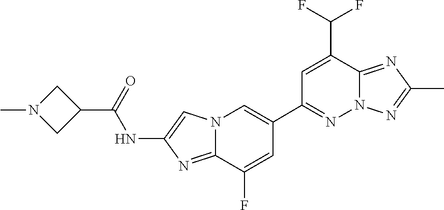 | N-(6-(8- (difluoromethyl)-2- methyl- [1,2,4]triazolo[1,5- b]pyridazin-6-yl)-8- fluoroimidazo[1,2- a]pyridin-2-yl)-1- methylazetidine-3- carboxamide |
| 311 | 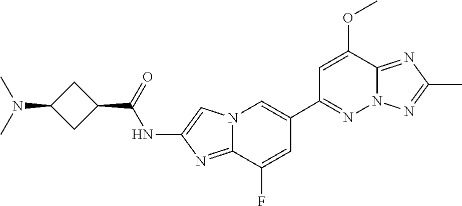 | (1s,3s)-3- (dimethylamino)-N-(8- fluoro-6-(8-methoxy-2- methyl- [1,2,4]triazolo[1,5- b]pyridazin-6- yl)imidazo[1,2- a]pyridin-2- yl)cyclobutane-1- carboxamide |
| 312 | 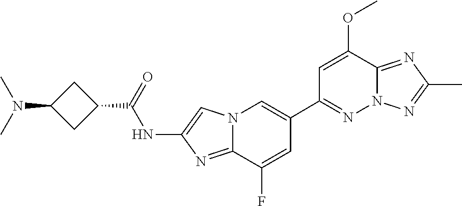 | (1r,3r)-3- (dimethylamino)-N-(8- fluoro-6-(8-methoxy-2- methyl- [1,2,4]triazolo[1,5- b]pyridazin-6- yl)imidazo[1,2- a]pyridin-2- yl)cyclobutane-1- carboxamide |
| 313 | 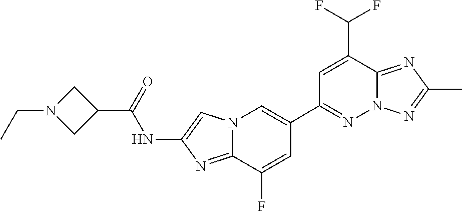 [SMILES: CCN1CC(C(=O)Nc2cn3cc(-c4cc(C(F)F)c5nc(C)nn5n4)cc(F)c3n2)C1] | N-(6-(8- (difluoromethyl)-2- methyl- [1,2,4]triazolo[1,5- b]pyridazin-6-yl)-8- fluoroimidazo[1,2- a]pyridin-2-yl)-1- ethylazetidine-3- carboxamide |
| 314 | 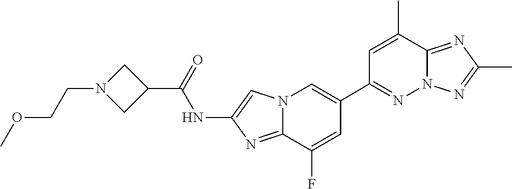 | N-(6-(2,8-dimethyl- [1,2,4]triazolo[1,5- b]pyridazin-6-yl)-8- fluoroimidazo[1,2- a]pyridin-2-yl)-1-(2- methoxyethyl)azetidine- 3-carboxamide |
| 315 | 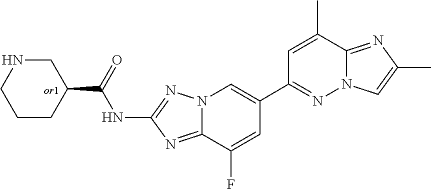 [SMILES: Cc1cn2nc(-c3cc(F)c4nc(NC(=O)[C@H]5CCCNC5)nn4c3)cc(C)c2n1] | rel-(R)-N-(6-(2,8- dimethylimidazo[1,2- b]pyridazin-6-yl)-8- fluoro- [1,2,4]triazolo[1,5- a]pyridin-2- yl)piperidine-3- carboxamide |
| 316 | 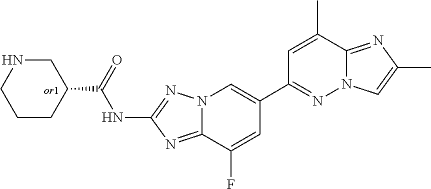 [SMILES: Cc1cn2nc(-c3cc(F)c4nc(NC(=O)[C@@H]5CCCNC5)nn4c3)cc(C)c2n1] | rel-(R)-N-(6-(2,8- dimethylimidazo[1,2- b]pyridazin-6-yl)-8- fluoro- [1,2,4]triazolo[1,5- a]pyridin-2- yl)piperidine-3- carboxamide |
| 317 | 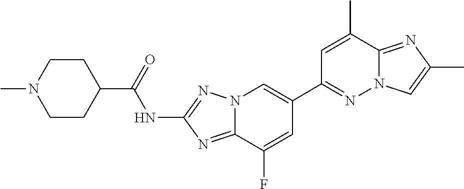 | N-(6-(2,8- dimethylimidazo[1,2- b]pyridazin-6-yl)-8- fluoro- [1,2,4]triazolo[1,5- a]pyridin-2-yl)-1- methylpiperidine-4- carboxamide |
| 318 | 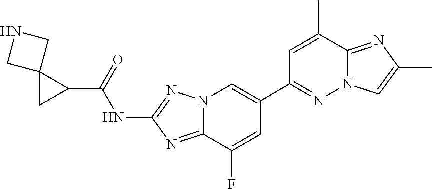 [SMILES: Cc1cn2nc(-c3cc(F)c4nc(NC(=O)C5CC56CNC6)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-5- azaspiro[2.3]]hexane-1- carboxamide |
| 319 | 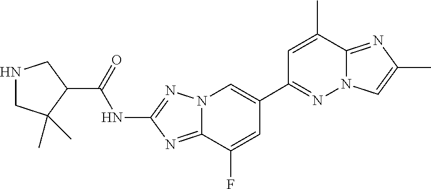 [SMILES: Cc1cn2nc(-c3cc(F)c4nc(NC(=O)C5CNCC5(C)C)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-4,4- dimethylpyrrolidine-3- carboxamide |
| 320 | 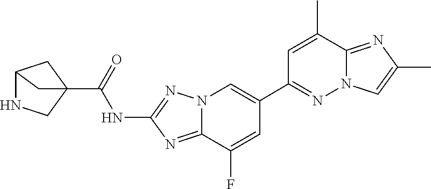 [SMILES: Cc1cn2nc(-c3cc(F)c4nc(NC(=O)C56CNC(C5)C6)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-2- azabicyclo[2.1.1]hexane- 4-carboxamide |
| 321 | 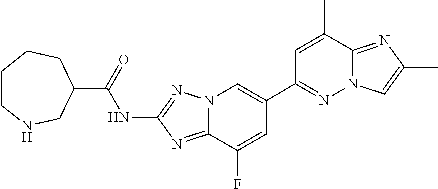 [SMILES: Cc1cn2nc(-c3cc(F)c4nc(NC(=O)C5CCCCNC5)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)azepane- 3-carboxamide |
| 322 | 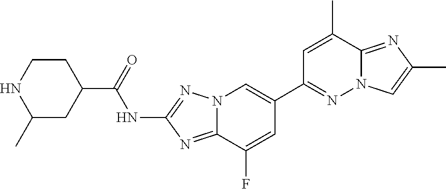 [SMILES: Cc1cn2nc(-c3cc(F)c4nc(NC(=O)C5CCNC(C)C5)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-2- methylpiperidine-4- carboxamide |
| 323 | 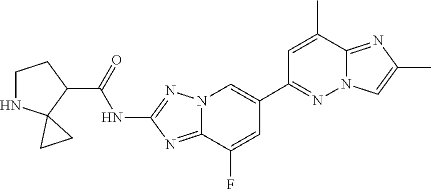 [SMILES: Cc1cn2nc(-c3cc(F)c4nc(NC(=O)C5CCNC56CC6)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-4- azaspiro[2.4]heptane-7- carboxamide |
| 324 | 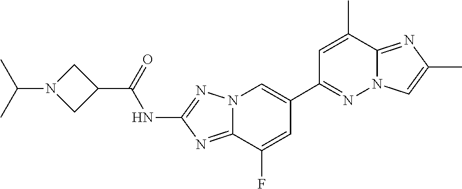 [SMILES: Cc1cn2nc(-c3cc(F)c4nc(NC(=O)C5CN(C(C)C)C5)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-1- isopropylazetidine-3- carboxamide |
| 325 | 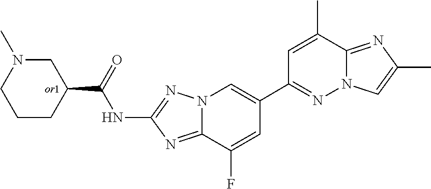 [SMILES: Cc1cn2nc(-c3cc(F)c4nc(NC(=O)[C@H]5CCCN(C)C5)nn4c3)cc(C)c2n1] | rel-(R)-N-(6-(2,8- dimethylimidazo[1,2- b]pyridazin-6-yl)-8- fluoro- [1,2,4]triazolo[1,5- a]pyridin-2-yl)-1- methylpiperidine-3- carboxamide |
| 326 | 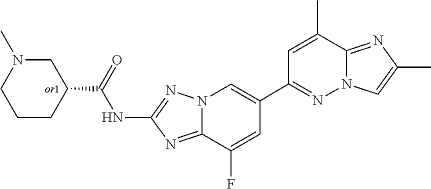 [SMILES: Cc1cn2nc(-c3cc(F)c4nc(NC(=O)[C@@H]5CCCN(C)C5)nn4c3)cc(C)c2n1] | rel-(R)-N-(6-(2,8- dimethylimidazo[1,2- b]pyridazin-6-yl)-8- fluoro- [1,2,4]triazolo[1,5- a]pyridin-2-yl)-1- methylpiperidine-3- carboxamide |
| 327 | 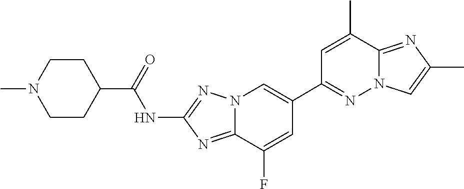 | N-(6-(2,8- dimethylimidazo[1,2- b]pyridazin-6-yl)-8- fluoro- [1,2,4]triazolo[1,5- a]pyridin-2-yl)-1- methylpiperidine-4- carboxamide |
| 328 | 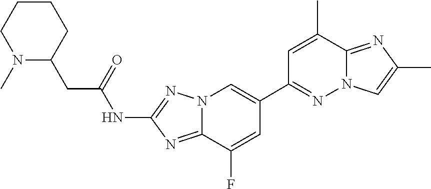 [SMILES: Cc1cn2nc(-c3cc(F)c4nc(NC(=O)CC5CCCCN5C)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-2-(1- methylpiperidin-2- yl)acetamide |
| 329 | 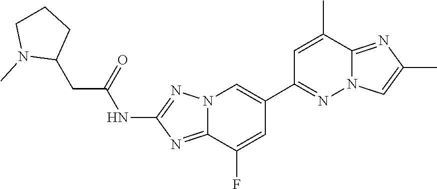 | N-(6-(2,8- dimethylimidazo[1,2- b]pyridazin-6-yl)-8- fluoro- [1,2,4]triazolo[1,5- a]pyridin-2-yl)-2-(1- methylpyrrolidin-2- yl)acetamide |
| 330 | 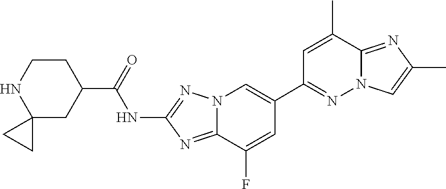 [SMILES: Cc1cn2nc(-c3cc(F)c4nc(NC(=O)C5CCNC6(CC6)C5)nn4c3)cc(C)c2n1] | N-(6-(2,8- dimethylimidazo[1,2- b]pyridazin-6-yl)-8- fluoro- [1,2,4]triazolo[1,5- a]pyridin-2-yl)-4- azaspiro[2.5]octane-7- carboxamide |
| 129 | 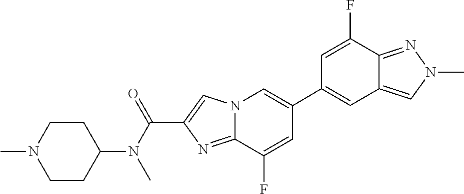 | 8-fluoro-6-(7-fluoro-2- methyl-2H-indazol-5- yl)-N-methyl-N-(1- methylpiperidin-4- yl)imidazo[1,2- a]pyridine-2- carboxamide |
| 331 | 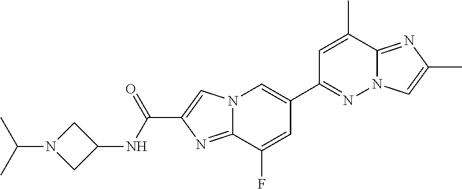 [SMILES: Cc1cn2nc(-c3cc(F)c4nc(C(=O)NC5CN(C(C)C)C5)cn4c3)cc(C)c2n1] | 6-(2,8- dimethylimidazo[1,2- b]pyridazin-6-yl)-8- fluoro-N-(1- isopropylazetidin-3- yl)imidazo[1,2- a]pyridine-2- carboxamide |
| 332 | 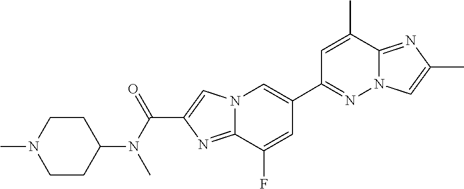 | 6-(2,8- dimethylimidazo[1,2- b]pyridazin-6-yl)-8- fluoro-N-methyl-N-(1- methylpiperidin-4- yl)imidazo[1,2- a]pyridine-2- carboxamide |
| 333 | 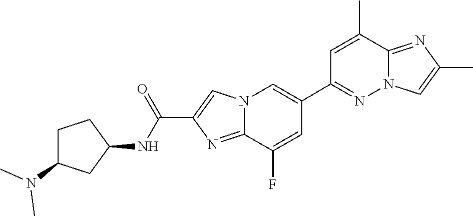 | N-((1R,3S)-3- (dimethylamino)cyclope ntyl)-6-(2,8- dimethylimidazo[1,2- b]pyridazin-6-yl)-8- fluoroimidazo[1,2- a]pyridine-2- carboxamide |
| 334 | 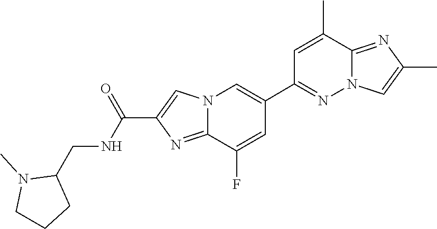 | 6-(2,8- dimethylimidazo[1,2- b]pyridazin-6-yl)-8- fluoro-N-((1- methylpyrrolidin-2- yl)methyl)imidazo[1,2- a]pyridine-2- carboxamide |
| 335 | 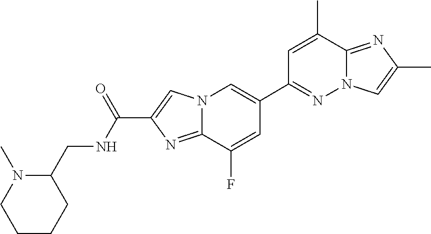 [SMILES: Cc1cn2nc(-c3cc(F)c4nc(C(=O)NCC5CCCCN5C)cn4c3)cc(C)c2n1] | 6-(2,8- dimethylimidazo[1,2- b]pyridazin-6-yl)-8- fluoro-N-((1- methylpiperidin-2- yl)methyl)imidazo[1,2- a]pyridine-2- carboxamide |
| 336 | 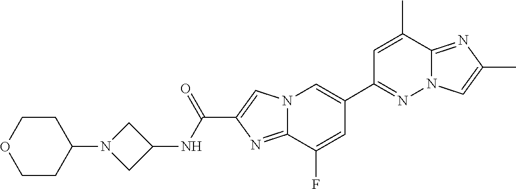 [SMILES: Cc1cn2nc(-c3cc(F)c4nc(C(=O)NC5CN(C6CCOCC6)C5)cn4c3)cc(C)c2n1] | 6-(2,8- dimethylimidazo[1,2- b]pyridazin-6-yl)-8- fluoro-N-(1-(tetrahydro- 2H-pyran-4-yl)azetidin- 3-yl)imidazo[1,2- a]pyridine-2- carboxamide |
| 337 | 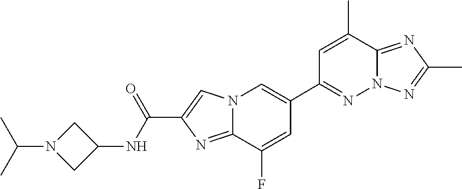 [SMILES: Cc1nc2c(C)cc(-c3cc(F)c4nc(C(=O)NC5CN(C(C)C)C5)cn4c3)nn2n1] | 6-(2,8-dimethyl- [1,2,4]triazolo[1,5- b]pyridazin-6-yl)-8- fluoro-N-(1- isopropylazetidin-3- yl)imidazo[1,2- a]pyridine-2- carboxamide |
| 338 | 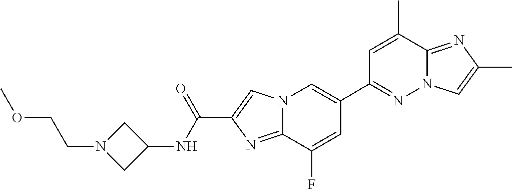 | 6-(2,8- dimethylimidazo[1,2- b]pyridazin-6-yl)-8- fluoro-N-(1-(2- methoxyethyl)azetidin- 3-yl)imidazo[1,2- a]pyridine-2- carboxamide |
| 339 | 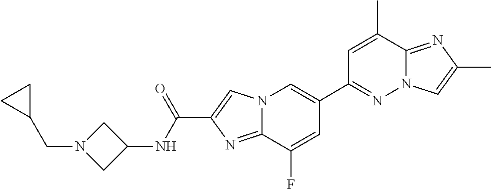 [SMILES: Cc1cn2nc(-c3cc(F)c4nc(C(=O)NC5CN(CC6CC6)C5)cn4c3)cc(C)c2n1] | N-(1- (cyclopropylmethyl)azet idin-3-yl)-6-(2,8- dimethylimidazo[1,2- b]pyridazin-6-yl)-8- fluoroimidazo[1,2- a]pyridine-2- carboxamide |
| 340 | 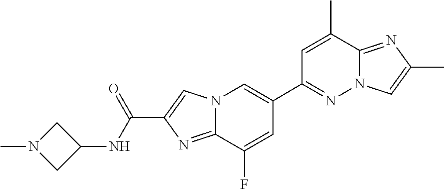 | 6-(2,8- dimethylimidazo[1,2- b]pyridazin-6-yl)-8- fluoro-N-(1- methylazetidin-3- yl)imidazo[1,2- a]pyridine-2- carboxamide |
| 341 | 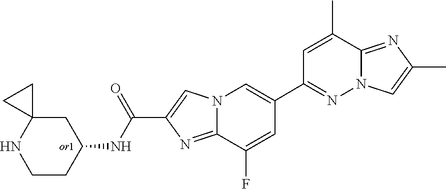 [SMILES: Cc1cn2nc(-c3cc(F)c4nc(C(=O)N[C@@H]5CCNC6(CC6)C5)cn4c3)cc(C)c2n1] | rel-(R)-6-(2,8- dimethylimidazo[1,2- b]pyridazin-6-yl)-8- fluoro-N-(4- azaspiro[2.5]octan-7- yl)imidazo[1,2- a]pyridine-2- carboxamide |
| 342 | 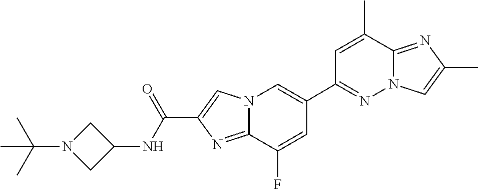 [SMILES: Cc1cn2nc(-c3cc(F)c4nc(C(=O)NC5CN(C(C)(C)C)C5)cn4c3)cc(C)c2n1] | N-(1-(tert- butyl)azetidin-3-yl)-6- (2,8- dimethylimidazo[1,2- b]pyridazin-6-yl)-8- fluoroimidazo[1,2- a]pyridine-2- carboxamide |
| 343 | 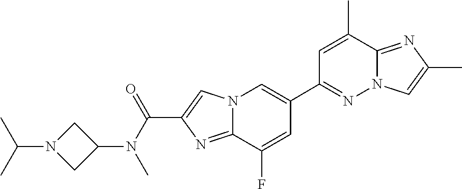 [SMILES: Cc1cn2nc(-c3cc(F)c4nc(C(=O)N(C)C5CN(C(C)C)C5)cn4c3)cc(C)c2n1] | 6-(2,8- dimethylimidazo[1,2- b]pyridazin-6-yl)-8- fluoro-N-(1- isopropylazetidin-3-yl)- N-methylimidazo[1,2- a]pyridine-2- carboxamide |
| 344 | 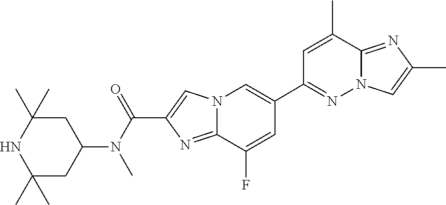 [SMILES: Cc1cn2nc(-c3cc(F)c4nc(C(=O)N(C)C5CC(C)(C)NC(C)(C)C5)cn4c3)cc(C)c2n1] | 6-(2,8- dimethylimidazo[1,2- b]pyridazin-6-yl)-8- fluoro-N-methyl-N- (2,2,6,6- tetramethylpiperidin-4- yl)imidazo[1,2- a]pyridine-2- carboxamide |
| 345 | 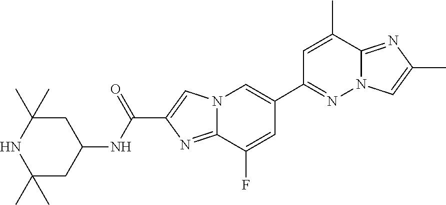 [SMILES: Cc1cn2nc(-c3cc(F)c4nc(C(=O)NC5CC(C)(C)NC(C)(C)C5)cn4c3)cc(C)c2n1] | 6-(2,8- dimethylimidazo[1,2- b]pyridazin-6-yl)-8- fluoro-N-(2,2,6,6- tetramethylpiperidin-4- yl)imidazo[1,2- a]pyridine-2- carboxamide |
| 346 | 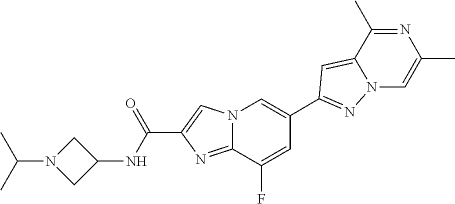 [SMILES: Cc1cn2nc(-c3cc(F)c4nc(C(=O)NC5CN(C(C)C)C5)cn4c3)cc2c(C)n1] | 6-(4,6- dimethylpyrazolo[1,5- a]pyrazin-2-yl)-8- fluoro-N-(1- isopropylazetidin-3- yl)imidazo[1,2- a]pyridine-2- carboxamide |
| 347 | 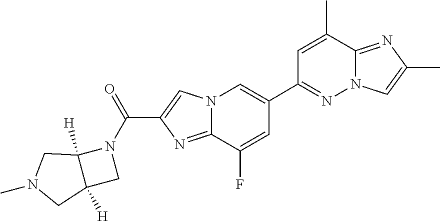 | (6-(2,8- dimethylimidazo[1,2- b]pyridazin-6-yl)-8- fluoroimidazo[1,2- a]pyridin-2-yl)((1R,5S)- 3-methyl-3,6- diazabicyclo[3.2.0] heptan-6-yl)methanone |
| 348 | 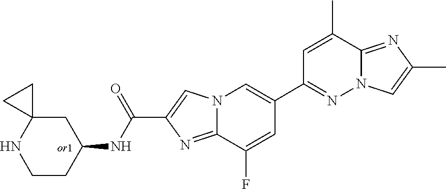 [SMILES: Cc1cn2nc(-c3cc(F)c4nc(C(=O)N[C@H]5CCNC6(CC6)C5)cn4c3)cc(C)c2n1] | rel-(R)-6-(2,8- dimethylimidazo[1,2- b]pyridazin-6-yl)-8- fluoro-N-(4- azaspiro[2.5]octan-7- yl)imidazo[1,2- a]pyridine-2- carboxamide |
| 349 | 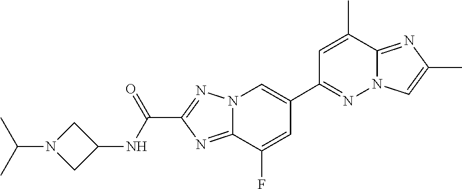 [SMILES: Cc1cn2nc(-c3cc(F)c4nc(C(=O)NC5CN(C(C)C)C5)nn4c3)cc(C)c2n1] | 6-(2,8- dimethylimidazo[1,2- b]pyridazin-6-yl)-8- fluoro-N-(1- isopropylazetidin-3-yl)- [1,2,4]triazolo[1,5- a]pyridine-2- carboxamide |
| 130 | 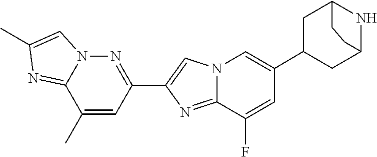 [SMILES: Cc1cn2nc(-c3cn4cc(C5CC6CCC(C5)N6)cc(F)c4n3)cc(C)c2n1] | 6-(6-(8- azabicyclo[3.2.1]]octan- 3-yl)-8- fluoroimidazo[1,2- a]pyridin-2-yl)-2,8- dimethylimidazo[1,2- b]pyridazine |
| 131 | 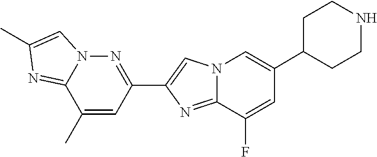 [SMILES: Cc1cn2nc(-c3cn4cc(C5CCNCC5)cc(F)c4n3)cc(C)c2n1] | 6-(8-fluoro-6-(piperidin- 4-yl)imidazo[1,2- a]pyridin-2-yl)-2,8- dimethylimidazo[1,2- b]pyridazine |
| 132 | 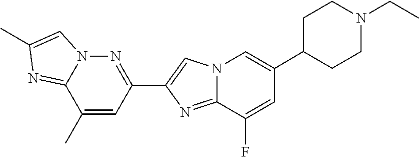 [SMILES: CCN1CCC(c2cc(F)c3nc(-c4cc(C)c5nc(C)cn5n4)cn3c2)CC1] | 6-(6-(1-ethylpiperidin-4- yl)-8-fluoroimidazo[1,2- a]pyridin-2-yl)-2,8- dimethylimidazo[1,2- b]]pyridazine |
| 133 | 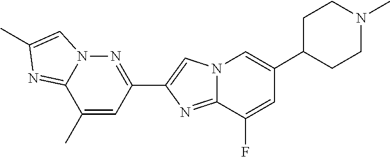 | 6-(8-fluoro-6-(1- methylpiperidin-4- yl)imidazo[1,2- a]pyridin-2-yl)-2,8- dimethylimidazo[1,2- b]pyridazine |
| 134 | 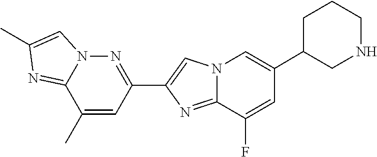 [SMILES: Cc1cn2nc(-c3cn4cc(C5CCCNC5)cc(F)c4n3)cc(C)c2n1] | 6-(8-fluoro-6-(piperidin- 3-yl)imidazo[1,2- a]pyridin-2-yl)-2,8- dimethylimidazo[1,2- b]pyridazine |
| 135 | 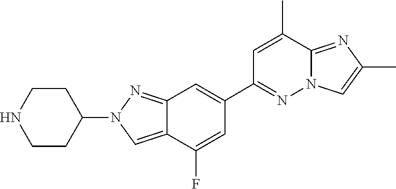 [SMILES: Cc1cn2nc(-c3cc(F)c4cn(C5CCNCC5)nc4c3)cc(C)c2n1] | 6-(4-fluoro-2-(piperidin- 4-yl)-2H-indazol-6-yl)- 2,8- dimethylimidazo[1,2- b]pyridazine |
| 136 | 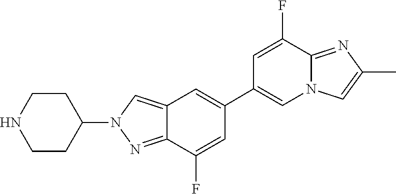 [SMILES: Cc1cn2cc(-c3cc(F)c4nn(C5CCNCC5)cc4c3)cc(F)c2n1] | 7-fluoro-5-(8-fluoro-2- methylimidazo[1,2- a]pyridin-6-yl)-2- (piperidin-4-yl)-2H- indazole |
| 350 | 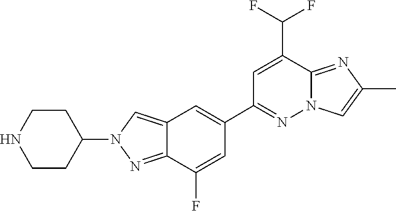 [SMILES: Cc1cn2nc(-c3cc(F)c4nn(C5CCNCC5)cc4c3)cc(C(F)F)c2n1] | 8-(difluoromethyl)-6-(7- fluoro-2-(piperidin-4- yl)-2H-indazol-5-yl)-2- methylimidazo[1,2- b]pyridazine |
| 351 | 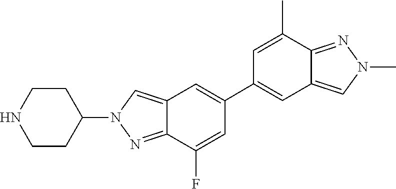 [SMILES: Cc1cc(-c2cc(F)c3nn(C4CCNCC4)cc3c2)cc2cn(C)nc12] | 7-fluoro-2′,7′-dimethyl- 2-(piperidin-4-yl)- 2H,2′H-5,5′-biindazole |
| 352 | 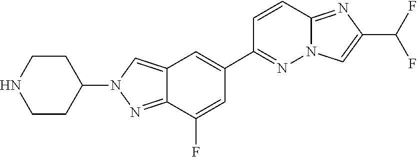 [SMILES: Fc1cc(-c2ccc3nc(C(F)F)cn3n2)cc2cn(C3CCNCC3)nc12] | 2-(difluoromethyl)-6-(7- fluoro-2-(piperidin-4- yl)-2H-indazol-5- yl)imidazo[1,2- b]pyridazine |
| 353 | 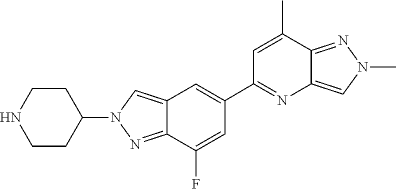 [SMILES: Cc1cc(-c2cc(F)c3nn(C4CCNCC4)cc3c2)nc2cn(C)nc12] | 5-(7-fluoro-2-(piperidin- 4-yl)-2H-indazol-5-yl)- 2,7-dimethyl-2H- pyrazolo[4,3-b]pyridine |
| 354 | 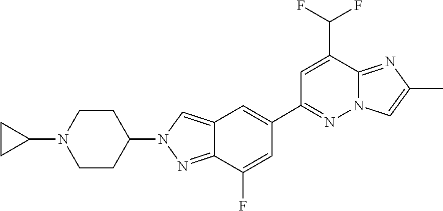 [SMILES: Cc1cn2nc(-c3cc(F)c4nn(C5CCN(C6CC6)CC5)cc4c3)cc(C(F)F)c2n1] | 6-(2-(1- cyclopropylpiperidin-4- yl)-7-fluoro-2H-indazol- 5-yl)-8- (difluoromethyl)-2- methylimidazo[1,2- b]pyridazine |
| 355 | 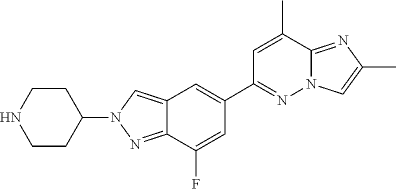 [SMILES: Cc1cn2nc(-c3cc(F)c4nn(C5CCNCC5)cc4c3)cc(C)c2n1] | 6-(7-fluoro-2-(piperidin- 4-yl)-2H-indazol-5-yl)- 2,8- dimethylimidazo[1,2- b]pyridazine |
| 356 | 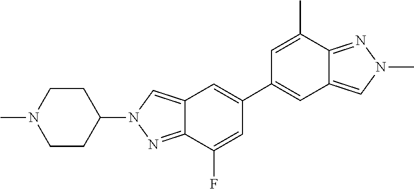 | 7-fluoro-2′,7′-dimethyl- 2-(1-methylpiperidin-4- yl)-2H,2′H-5,5′- biindazole |
| 357 | 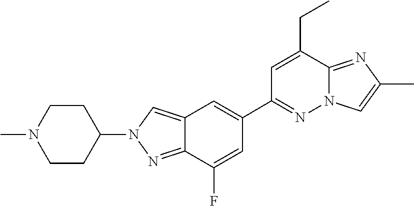 | 8-ethyl-6-(7-fluoro-2-(1- methylpiperidin-4-yl)- 2H-indazol-5-yl)-2- methylimidazo[1,2- b]pyridazine |
| 358 | 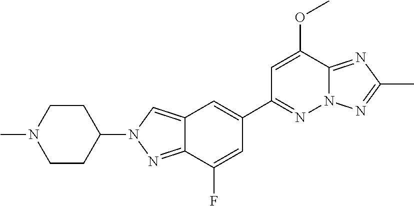 | 6-(7-fluoro-2-(1- methylpiperidin-4-yl)- 2H-indazol-5-yl)-8- methoxy-2-methyl- [1,2,4]triazolo[1,5- b]pyridazine |
| 359 | 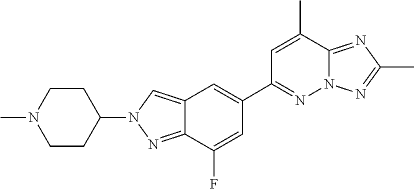 | 6-(7-fluoro-2-(1- methylpiperidin-4-yl)- 2H-indazol-5-yl)-2,8- dimethyl- [1,2,4]triazolo[1,5- b]pyridazine |
| 360 | 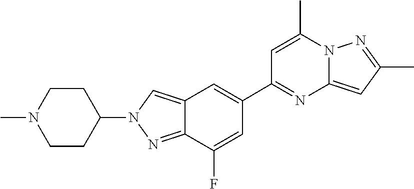 | 5-(7-fluoro-2-(1- methylpiperidin-4-yl)- 2H-indazol-5-yl)-2,7- dimethylpyrazolo[1,5- a]pyrimidine |
| 361 | 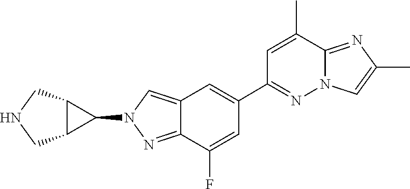 [SMILES: Cc1cn2nc(-c3cc(F)c4nn([C@H]5[C@@H]6CNC[C@@H]65)cc4c3)cc(C)c2n1] | 6-(2-((1R,5S,6s)-3- azabicyclo[3.1.0]hexan- 6-yl)-7-fluoro-2H- indazol-5-yl)-2,8- dimethylimidazo[1,2- b]pyridazine |
| 362 | 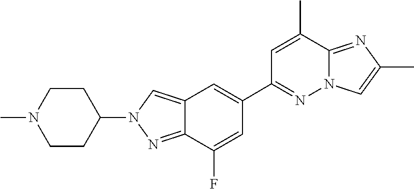 | 6-(7-fluoro-2-(1- methylpiperidin-4-yl)- 2H-indazol-5-yl)-2,8- dimethylimidazo[1,2- b]pyridazine |
| 363 | 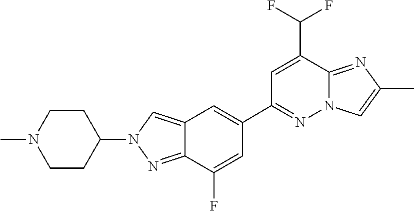 | 8-(difluoromethyl)-6-(7- fluoro-2-(1- methylpiperidin-4-yl)- 2H-indazol-5-yl)-2- methylimidazo[1,2- b]pyridazine |
| 364 | 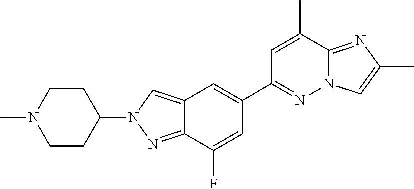 | 6-(7-fluoro-2-(1- methylpiperidin-4-yl)- 2H-indazol-5-yl)-2,8- dimethylimidazo[1,2- b]pyridazine |
| 365 | 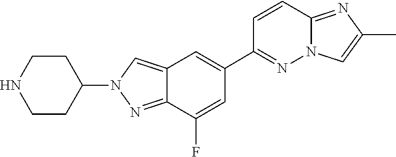 [SMILES: Cc1cn2nc(-c3cc(F)c4nn(C5CCNCC5)cc4c3)ccc2n1] | 6-(7-fluoro-2-(piperidin- 4-yl)-2H-indazol-5-yl)- 2-methylimidazo[1,2- b]pyridazine |
| 366 | 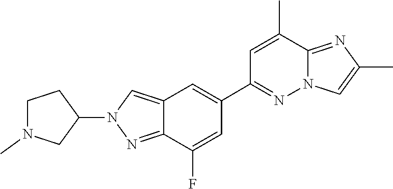 | 6-(7-fluoro-2-(1- methylpyrrolidin-3-yl)- 2H-indazol-5-yl)-2,8- dimethylimidazo[1,2- b]pyridazine |
| 367 | 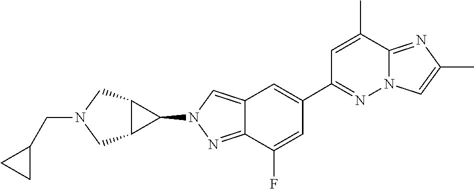 [SMILES: Cc1cn2nc(-c3cc(F)c4nn([C@H]5[C@@H]6CN(CC7CC7)C[C@@H]65)cc4c3)cc(C)c2n1] | 6-(2-((1R,5S,6s)-3- (cyclopropylmethyl)-3- azabicyclo[3.1.0]hexan- 6-yl)-7-fluoro-2H- indazol-5-yl)-2,8- dimethylimidazo[1,2- b]pyridazine |
| 368 | 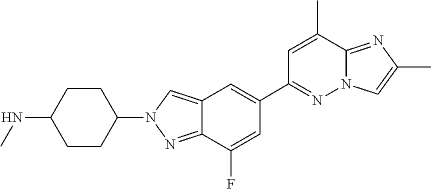 | 4-(5-(2,8- dimethylimidazo[1,2- b]pyridazin-6-yl)-7- fluoro-2H-indazol-2-yl)- N-methylcyclohexan-1- amine |
| 369 | 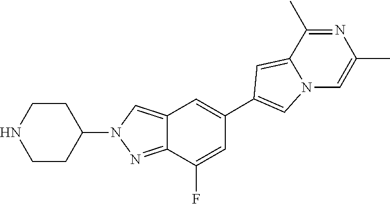 [SMILES: Cc1cn2cc(-c3cc(F)c4nn(C5CCNCC5)cc4c3)cc2c(C)n1] | 5-(1,3- dimethylpyrrolo[1,2- a]pyrazin-7-yl)-7- fluoro-2-(piperidin-4- yl)-2H-indazole |
| 370 | 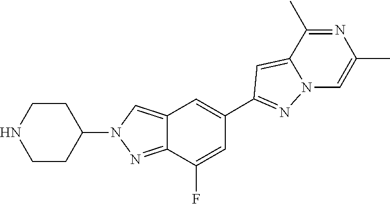 [SMILES: Cc1cn2nc(-c3cc(F)c4nn(C5CCNCC5)cc4c3)cc2c(C)n1] | 2-(7-fluoro-2-(piperidin- 4-yl)-2H-indazol-5-yl)- 4,6- dimethylpyrazolo[1,5- a]pyrazine |
| 371 | 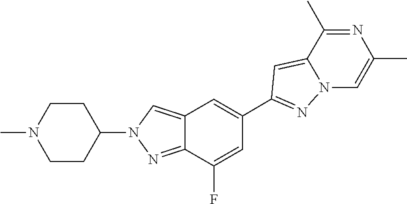 | 2-(7-fluoro-2-(1- methylpiperidin-4-yl)- 2H-indazol-5-yl)-4,6- dimethylpyrazolo[1,5- a]pyrazine |
| 372 | 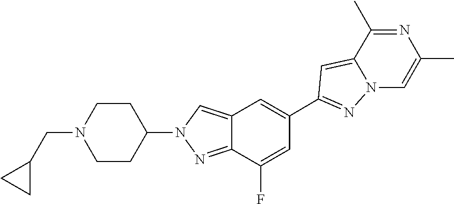 [SMILES: Cc1cn2nc(-c3cc(F)c4nn(C5CCN(CC6CC6)CC5)cc4c3)cc2c(C)n1] | 2-(2-(1- (cyclopropylmethyl) piperidin-4-yl)-7- fluoro-2H- indazol-5-yl)-4,6- dimethylpyrazolo[1,5- a]pyrazine |
| 373 | 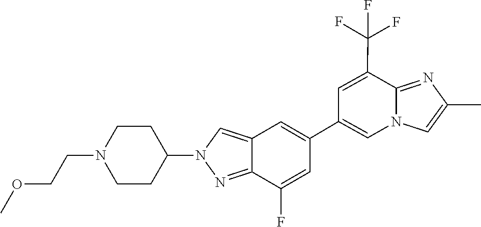 | 6-(7-fluoro-2-(1-(2- methoxyethyl)piperidin- 4-yl)-2H-indazol-5-yl)- 2-methyl-8- (trifluoromethyl)imidazo [1,2-b]pyridazine |
| 137 | 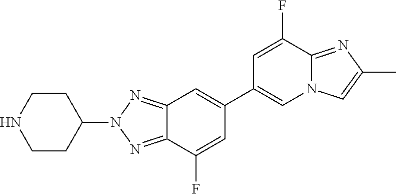 [SMILES: Cc1cn2cc(-c3cc(F)c4nn(C5CCNCC5)nc4c3)cc(F)c2n1] | 4-fluoro-6-(8-fluoro-2- methylimidazo[1,2- a]pyridin-6-yl)-2- (piperidin-4-yl)-2H- benzo[d][1,2,3]triazole |
| 138 | 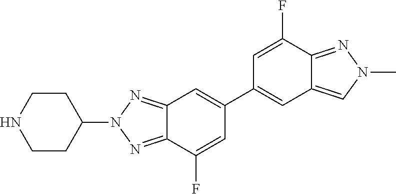 [SMILES: Cn1cc2cc(-c3cc(F)c4nn(C5CCNCC5)nc4c3)cc(F)c2n1] | 4-fluoro-6-(7-fluoro-2- methyl-2H-indazol-5- yl)-2-(piperidin-4-yl)- 2H- benzo[d][1,2,3]triazole |
| 139 | 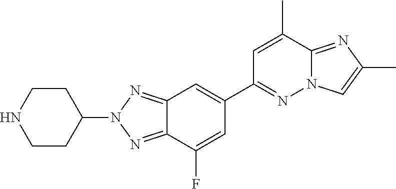 [SMILES: Cc1cn2nc(-c3cc(F)c4nn(C5CCNCC5)nc4c3)cc(C)c2n1] | 6-(2,8- dimethylimidazo[1,2- b]pyridazin-6-yl)-4- fluoro-2-(piperidin-4- yl)-2H- benzo[d][1,2,3]triazole |
| 374 | 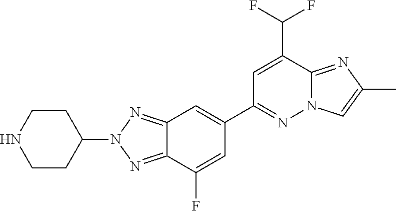 [SMILES: Cc1cn2nc(-c3cc(F)c4nn(C5CCNCC5)nc4c3)cc(C(F)F)c2n1] | 6-(8-(difluoromethyl)-2- methylimidazo[1,2- b]pyridazin-6-yl)-4- fluoro-2-(piperidin-4- yl)-2H- benzo[d][1,2,3]triazole |
| 375 | 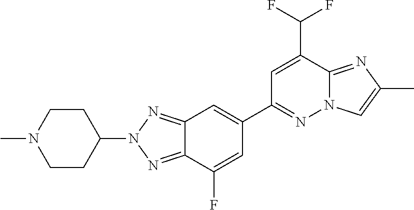 | 6-(8-(difluoromethyl)-2- methylimidazo[1,2- b]pyridazin-6-yl)-4- fluoro-2-(1- methylpiperidin-4-yl)- 2H- benzo[d][1,2,3]triazole |
| 376 | 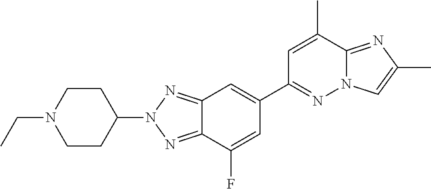 [SMILES: CCN1CCC(n2nc3cc(-c4cc(C)c5nc(C)cn5n4)cc(F)c3n2)CC1] | 6-(2,8- dimethylimidazo[1,2- b]pyridazin-6-yl)-2-(1- ethylpiperidin-4-yl)-4- fluoro-2H- benzo[d][1,2,3]triazole |
| 377 | 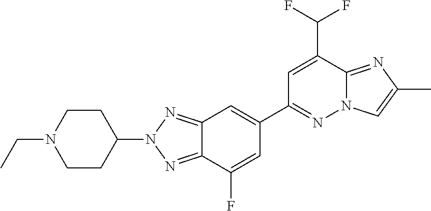 [SMILES: CCN1CCC(n2nc3cc(-c4cc(C(F)F)c5nc(C)cn5n4)cc(F)c3n2)CC1] | 6-(8-(difluoromethyl)-2- methylimidazo[1,2- b]pyridazin-6-yl)-2-(1- ethylpiperidin-4-yl)-4- fluoro-2H- benzo[d][1,2,3]triazole |
| 378 | 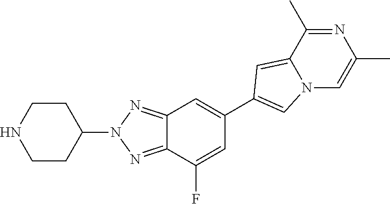 [SMILES: Cc1cn2cc(-c3cc(F)c4nn(C5CCNCC5)nc4c3)cc2c(C)n1] | 6-(1,3- dimethylpyrrolo[1,2- a]pyrazin-7-yl)-4- fluoro-2-(piperidin-4- yl)-2H- benzo[d][1,2,3]triazole |
| 379 | 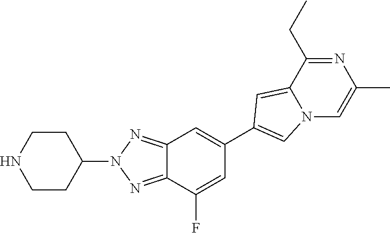 [SMILES: CCc1nc(C)cn2cc(-c3cc(F)c4nn(C5CCNCC5)nc4c3)cc12] | 6-(1-ethyl-3- methylpyrrolo[1,2- a]pyrazin-7-yl)-4- fluoro-2-(piperidin-4- yl)-2H- benzo[d][1,2,3]triazole |
| 380 | 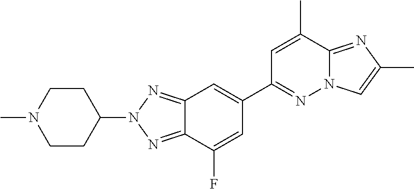 | 6-(2,8- dimethylimidazo[1,2- b]pyridazin-6-yl)-4- fluoro-2-(1- methylpiperidin-4-yl)- 2H- benzo[d][1,2,3]triazole |
| 381 | 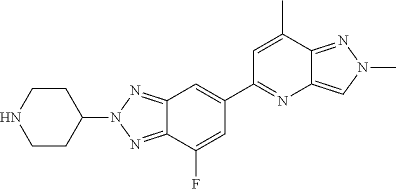 [SMILES: Cc1cc(-c2cc(F)c3nn(C4CCNCC4)nc3c2)nc2cn(C)nc12] | 6-(2,7-dimethyl-2H- pyrazolo[4,3-b]pyridin- 5-yl)-4-fluoro-2- (piperidin-4-yl)-2H- benzo[d][1,2,3]triazole |
| 382 | 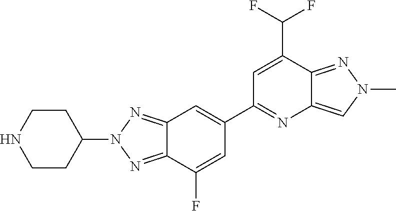 [SMILES: Cn1cc2nc(-c3cc(F)c4nn(C5CCNCC5)nc4c3)cc(C(F)F)c2n1] | 6-(7-(difluoromethyl)-2- methyl-2H-indazol-5- yl)-4-fluoro-2- (piperidin-4-yl)-2H- benzo[d][1,2,3]triazole |
| 383 | 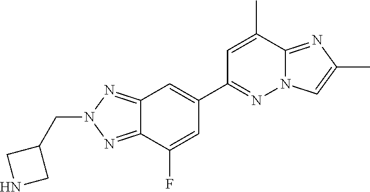 [SMILES: Cc1cn2nc(-c3cc(F)c4nn(CC5CNC5)nc4c3)cc(C)c2n1] | 2-(azetidin-3-ylmethyl)- 6-(2,8- dimethylimidazo[1,2- b]pyridazin-6-yl)-4- fluoro-2H- benzo[d][1,2,3]triazole |
| 384 | 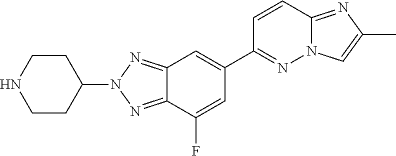 [SMILES: Cc1cn2nc(-c3cc(F)c4nn(C5CCNCC5)nc4c3)ccc2n1] | 4-fluoro-6-(2- methylimidazo[1,2- b]pyridazin-6-yl)-2- (piperidin-4-yl)-2H- benzo[d][1,2,3]triazole |
| 385 | 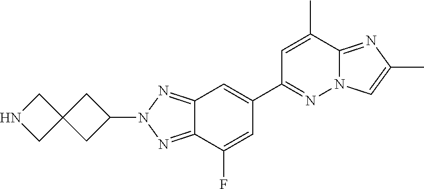 [SMILES: Cc1cn2nc(-c3cc(F)c4nn(C5CC6(CNC6)C5)nc4c3)cc(C)c2n1] | 6-(2,8- dimethylimidazo[1,2- b]pyridazin-6-yl)-4- fluoro-2-(2- azaspiro[3.3]heptan-6- yl)-2H- benzo[d][1,2,3]triazole |
| 386 | 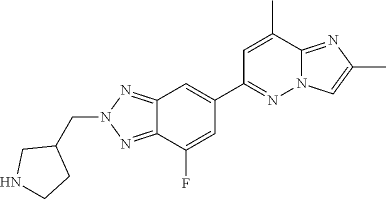 [SMILES: Cc1cn2nc(-c3cc(F)c4nn(CC5CCNC5)nc4c3)cc(C)c2n1] | 6-(2,8- dimethylimidazo[1,2- b]pyridazin-6-yl)-4- fluoro-2-(pyrrolidin-3- ylmethyl)-2H- benzo[d][1,2,3]triazole |
| 387 | 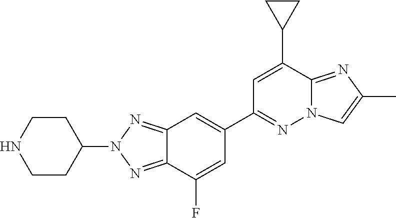 [SMILES: Cc1cn2nc(-c3cc(F)c4nn(C5CCNCC5)nc4c3)cc(C3CC3)c2n1] | 6-(8-cyclopropyl-2- methylimidazo[1,2- b]pyridazin-6-yl)-4- fluoro-2-(piperidin-4- yl)-2H- benzo[d][1,2,3]triazole |
| 388 | 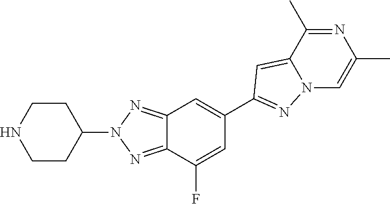 [SMILES: Cc1cn2nc(-c3cc(F)c4nn(C5CCNCC5)nc4c3)cc2c(C)n1] | 6-(4,6- dimethylpyrazolo[1,5- a]pyrazin-2-yl)-4- fluoro-2-(piperidin-4- yl)-2H- benzo[d][1,2,3]triazole |
| 389 | 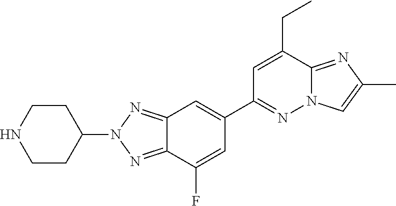 [SMILES: CCc1cc(-c2cc(F)c3nn(C4CCNCC4)nc3c2)nn2cc(C)nc12] | 6-(8-ethyl-2- methylimidazo[1,2- b]pyridazin-6-yl)-4- fluoro-2-(piperidin-4- yl)-2H- benzo[d][1,2,3]triazole |
| 390 | 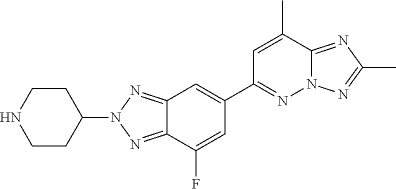 [SMILES: Cc1nc2c(C)cc(-c3cc(F)c4nn(C5CCNCC5)nc4c3)nn2n1] | 6-(7-fluoro-2-(piperidin- 4-yl)-2H- benzo[d][1,2,3]triazol-5- yl)-2,8-dimethyl- [1,2,4]triazolo[1,5- b]pyridazine |
| 391 | 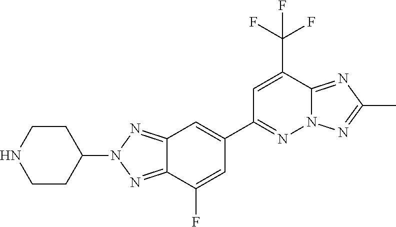 [SMILES: Cc1nc2c(C(F)(F)F)cc(-c3cc(F)c4nn(C5CCNCC5)nc4c3)nn2n1] | 6-(7-fluoro-2-(piperidin- 4-yl)-2H- benzo[d][1,2,3]triazol-5- yl)-2-methyl-8- (trifluoromethyl)- [1,2,4]triazolo[1,5- b]pyridazine |
| 392 | 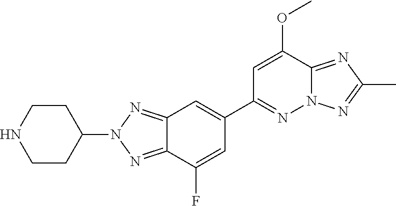 [SMILES: COc1cc(-c2cc(F)c3nn(C4CCNCC4)nc3c2)nn2nc(C)nc12] | 6-(7-fluoro-2-(piperidin- 4-yl)-2H- benzo[d][1,2,3]triazol-5- yl)-8-methoxy-2- methyl- [1,2,4]triazolo[1,5- b]pyridazine |
| 393 | 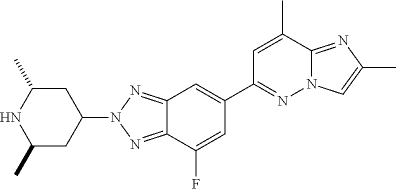 [SMILES: Cc1cn2nc(-c3cc(F)c4nn(C5C[C@@H](C)N[C@H](C)C5)nc4c3)cc(C)c2n1] | 6-(2,8- dimethylimidazo[1,2- b]pyridazin-6-yl)-2- ((2R,6R)-2,6- dimethylpiperidin-4-yl)- 4-fluoro-2H- benzo[d][1,2,3]triazole |
| 394 | 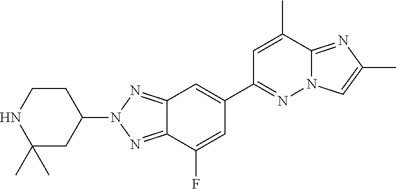 [SMILES: Cc1cn2nc(-c3cc(F)c4nn(C5CCNC(C)(C)C5)nc4c3)cc(C)c2n1] | 6-(2,8- dimethylimidazo[1,2- b]pyridazin-6-yl)-2-(2,2- dimethylpiperidin-4-yl)- 4-fluoro-2H- benzo[d][1,2,3]triazole |
| 395 | 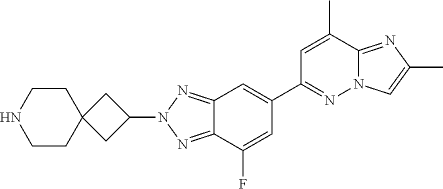 [SMILES: Cc1cn2nc(-c3cc(F)c4nn(C5CC6(CCNCC6)C5)nc4c3)cc(C)c2n1] | 6-(2,8- dimethylimidazo[1,2- b]pyridazin-6-yl)-4- fluoro-2-(7- azaspiro[3.5]nonan-2- yl)-2H- benzo[d][1,2,3]triazole |
| 396 | 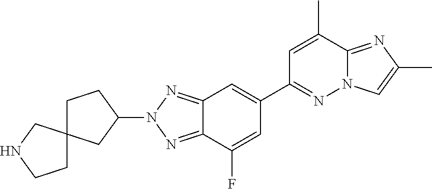 [SMILES: Cc1cn2nc(-c3cc(F)c4nn(C5CCC6(CCNC6)C5)nc4c3)cc(C)c2n1] | 6-(2,8- dimethylimidazo[1,2- b]pyridazin-6-yl)-4- fluoro-2-(2- azaspiro[4.4]nonan-7- yl)-2H- benzo[d][1,2,3]triazole |
| 397 | 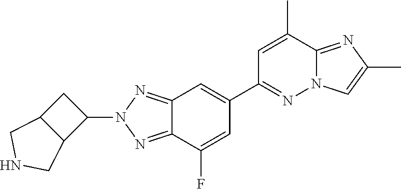 [SMILES: Cc1cn2nc(-c3cc(F)c4nn(C5CC6CNCC65)nc4c3)cc(C)c2n1] | 2-(3- azabicyclo[3.2.0]heptan- 6-yl)-6-(2,8- dimethylimidazo[1,2- b]pyridazin-6-yl)-4- fluoro-2H- benzo[d][1,2,3]triazole |
| 398 | 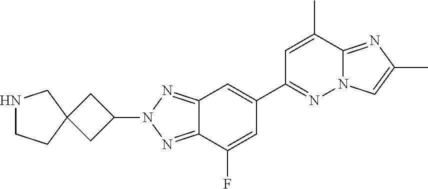 [SMILES: Cc1cn2nc(-c3cc(F)c4nn(C5CC6(CCNC6)C5)nc4c3)cc(C)c2n1] | 6-(2,8- dimethylimidazo[1,2- b]pyridazin-6-yl)-4- fluoro-2-(6- azaspiro[3.4]octan-2- yl)-2H- benzo[d][1,2,3]triazole |
| 399 | 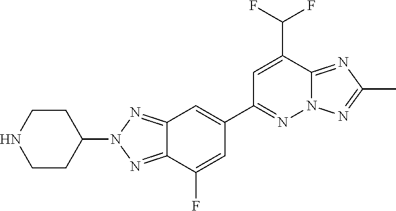 [SMILES: Cc1nc2c(C(F)F)cc(-c3cc(F)c4nn(C5CCNCC5)nc4c3)nn2n1] | 8-(difluoromethyl)-6-(7- fluoro-2-(piperidin-4- yl)-2H- benzo[d][1,2,3]triazol-5- yl)-2-methyl- [1,2,4]triazolo[1,5- b]pyridazine |
| 400 | 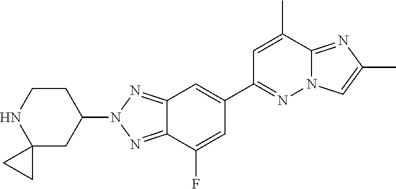 [SMILES: Cc1cn2nc(-c3cc(F)c4nn(C5CCNC6(CC6)C5)nc4c3)cc(C)c2n1] | 6-(2,8- dimethylimidazo[1,2- b]pyridazin-6-yl)-4- fluoro-2-(4- azaspiro[2.5]octan-7- yl)-2H- benzo[d][1,2,3]triazole |
| 401 | 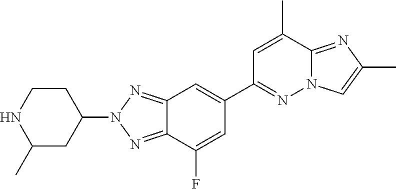 [SMILES: Cc1cn2nc(-c3cc(F)c4nn(C5CCNC(C)C5)nc4c3)cc(C)c2n1] | 6-(2,8- dimethylimidazo[1,2- b]pyridazin-6-yl)-4- fluoro-2-(2- methylpiperidin-4-yl)- 2H- benzo[d][1,2,3]triazole |
| 402 | 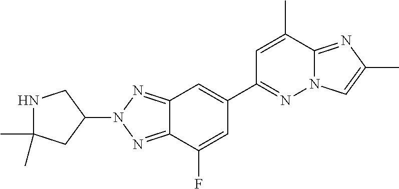 | 6-(2,8- dimethylimidazo[1,2- b]pyridazin-6-yl)-2-(5,5- dimethylpyrrolidin-3- yl)-4-fluoro-2H- benzo[d][1,2,3]triazole |
| 403 | 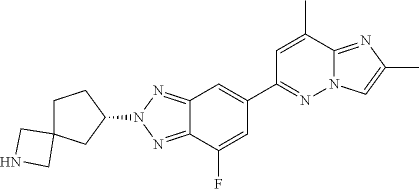 [SMILES: Cc1cn2nc(-c3cc(F)c4nn([C@H]5CCC6(CNC6)C5)nc4c3)cc(C)c2n1] | 2-[(6S)-2- azaspiro[3.4]octan-6- yl]-6-(2,8- dimethylimidazo[1,2- b]pyridazin-6-yl)-4- fluoro-benzotriazole |
| 404 | 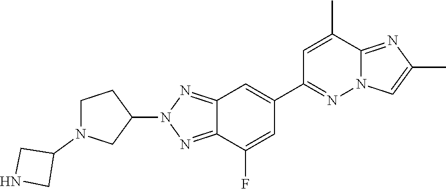 [SMILES: Cc1cn2nc(-c3cc(F)c4nn(C5CCN(C6CNC6)C5)nc4c3)cc(C)c2n1] | 2-(1-(azetidin-3- yl)pyrrolidin-3-yl)-6- (2,8- dimethylimidazo[1,2- b]pyridazin-6-yl)-4- fluoro-2H- benzo[d][1,2,3]triazole |
| 405 | 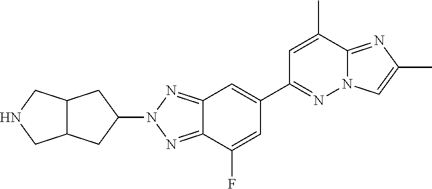 [SMILES: Cc1cn2nc(-c3cc(F)c4nn(C5CC6CNCC6C5)nc4c3)cc(C)c2n1] | 6-(2,8- dimethylimidazo[1,2- b]pyridazin-6-yl)-4- fluoro-2- (octahydrocyclopenta[c] pyrrol-5-yl)-2H- benzo[d][1,2,3]triazole |
| 406 | 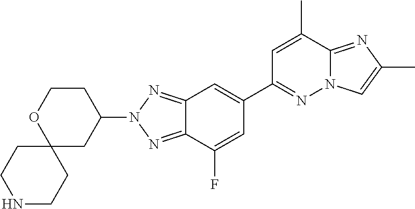 [SMILES: Cc1cn2nc(-c3cc(F)c4nn(C5CCOC6(CCNCC6)C5)nc4c3)cc(C)c2n1] | 4-(6-(2,8- dimethylimidazo[1,2- b]pyridazin-6-yl)-4- fluoro-2H- benzo[d][1,2,3]triazol-2- yl)-1-oxa-9- azaspiro[5.5]undecane |
| 407 | 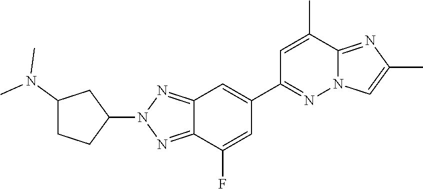 | 3-(6-(2,8- dimethylimidazo[1,2- b]pyridazin-6-yl)-4- fluoro-2H- benzo[d][1,2,3]triazol-2- yl)-N,N- dimethylcyclopentan-1- amine |
| 408 | 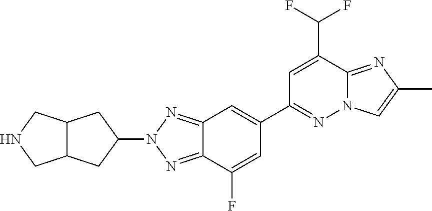 [SMILES: Cc1cn2nc(-c3cc(F)c4nn(C5CC6CNCC6C5)nc4c3)cc(C(F)F)c2n1] | 6-(8-(difluoromethyl)-2- methylimidazo[1,2- b]pyridazin-6-yl)-4- fluoro-2- (octahydrocyclopenta[c] pyrrol-5-yl)-2H- benzo[d][1,2,3]triazole |
| 409 | 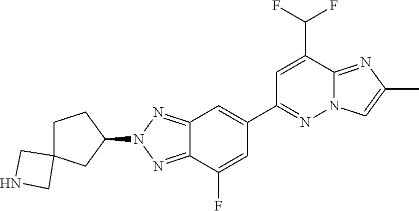 [SMILES: Cc1cn2nc(-c3cc(F)c4nn([C@@H]5CCC6(CNC6)C5)nc4c3)cc(C(F)F)c2n1] | (R)-6-(8- (difluoromethyl)-2- methylimidazo[1,2- b]pyridazin-6-yl)-4- fluoro-2-(2- azaspiro[3.4]octan-6- yl)-2H- benzo[d][1,2,3]triazole |
| 410 | 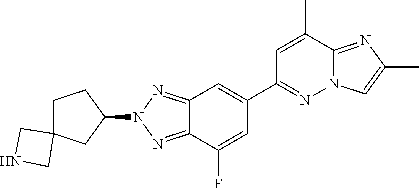 [SMILES: Cc1cn2nc(-c3cc(F)c4nn([C@@H]5CCC6(CNC6)C5)nc4c3)cc(C)c2n1] | (R)-6-(2,8- dimethylimidazo[1,2- b]pyridazin-6-yl)-4- fluoro-2-(2- azaspiro[3.4]octan-6- yl)-2H- benzo[d][1,2,3]triazole |
| 411 | 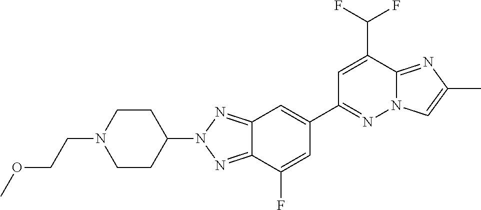 | 6-(8-(difluoromethyl)-2- methylimidazo[1,2- b]pyridazin-6-yl)-4- fluoro-2-(1-(2- methoxyethyl)piperidin- 4-yl)-2H- benzo[d][1,2,3]triazole |
| 412 | 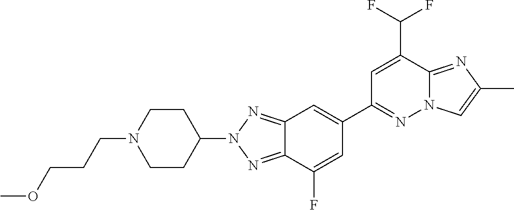 | 6-(8-(difluoromethyl)-2- methylimidazo[1,2- b]pyridazin-6-yl)-4- fluoro-2-(1-(3- methoxypropyl) piperidin-4-yl)-2H- benzo[d][1,2,3]triazole |
| 413 | 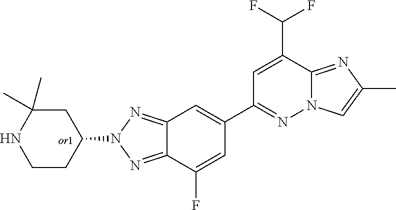 | rel-(R)-6-(8- (difluoromethyl)-2- methylimidazo[1,2- b]pyridazin-6-yl)-2-(2,2- dimethylpiperidin-4-yl)- 4-fluoro-2H- benzo[d][1,2,3]triazole |
| 414 | 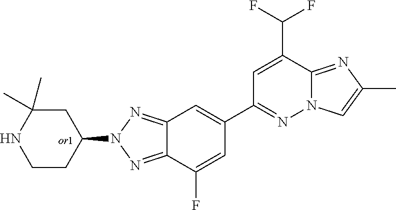 | rel-(R)-6-(8- (difluoromethyl)-2- methylimidazo[1,2- b]pyridazin-6-yl)-2-(2,2- dimethylpiperidin-4-yl)- 4-fluoro-2H- benzo[d][1,2,3]triazole |
| 415 | 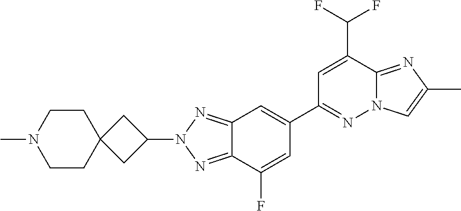 | 6-(8-(difluoromethyl)-2- methylimidazo[1,2- b]pyridazin-6-yl)-4- fluoro-2-(7-methyl-7- azaspiro[3.5]nonan-2- yl)-2H- benzo[d][1,2,3]triazole |
| 416 | 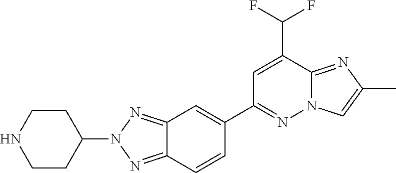 [SMILES: Cc1cn2nc(-c3ccc4nn(C5CCNCC5)nc4c3)cc(C(F)F)c2n1] | 5-(8-(difluoromethyl)-2- methylimidazo[1,2- b]pyridazin-6-yl)-2- (piperidin-4-yl)-2H- benzo[d][1,2,3]triazole |
| 140 | 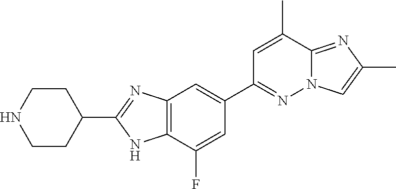 [SMILES: Cc1cn2nc(-c3cc(F)c4[nH]c(C5CCNCC5)nc4c3)cc(C)c2n1] | 6-(7-fluoro-2-(piperidin- 4-yl)-1H- benzo[d]imidazol-5-yl)- 2,8- dimethylimidazo[1,2- b]pyridazine |
| 141 | 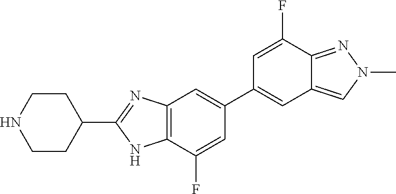 [SMILES: Cn1cc2cc(-c3cc(F)c4[nH]c(C5CCNCC5)nc4c3)cc(F)c2n1] | 7-fluoro-5-(7-fluoro-2- (piperidin-4-yl)-1H- benzo[d]imidazol-5-yl)- 2-methyl-2H-indazole |
| 142 | 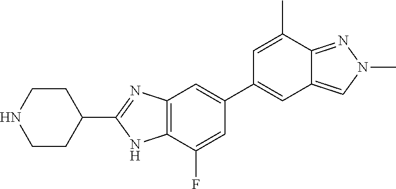 [SMILES: Cc1cc(-c2cc(F)c3[nH]c(C4CCNCC4)nc3c2)cc2cn(C)nc12] | 5-(7-fluoro-2-(piperidin- 4-yl)-1H- benzo[d]imidazol-5-yl)- 2,7-dimethyl-2H- indazole |
| 143 | 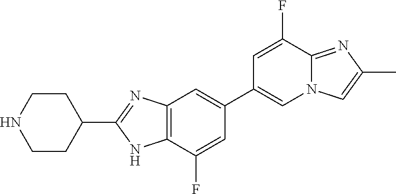 [SMILES: Cc1cn2cc(-c3cc(F)c4[nH]c(C5CCNCC5)nc4c3)cc(F)c2n1] | 7-fluoro-5-(8-fluoro-2- methylimidazo[1,2- a]pyridin-6-yl)-2- (piperidin-4-yl)-1H- benzo[d]imidazole |
| 144 | 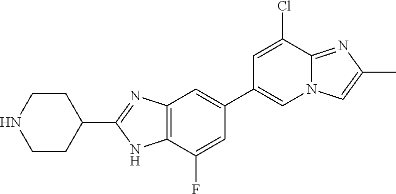 [SMILES: Cc1cn2cc(-c3cc(F)c4[nH]c(C5CCNCC5)nc4c3)cc(Cl)c2n1] | 5-(8-chloro-2- methylimidazo[1,2- a]pyridin-6-yl)-7-fluoro- 2-(piperidin-4-yl)-1H- benzo[d]imidazole |
| 145 | 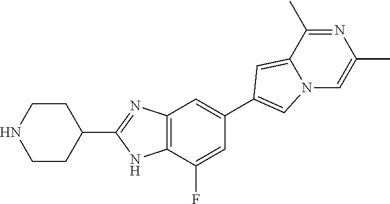 [SMILES: Cc1cn2cc(-c3cc(F)c4[nH]c(C5CCNCC5)nc4c3)cc2c(C)n1] | 5-(1,3- dimethylpyrrolo[1,2- a]pyrazin-7-yl)-7- fluoro-2-(piperidin-4- yl)-1H- benzo[d]imidazole |
| 146 | 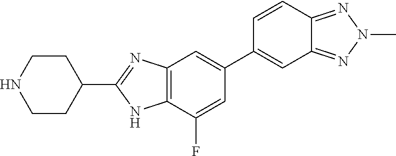 [SMILES: Cn1nc2ccc(-c3cc(F)c4[nH]c(C5CCNCC5)nc4c3)cc2n1] | 5-(7-fluoro-2-(piperidin- 4-yl)-1H- benzo[d]imidazol-5-yl)- 2-methyl-2H- benzo[d][1,2,3]triazole |
| 147 | 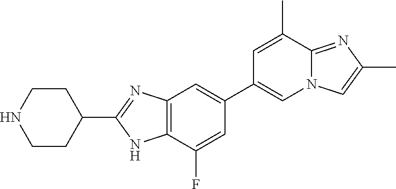 [SMILES: Cc1cn2cc(-c3cc(F)c4[nH]c(C5CCNCC5)nc4c3)cc(C)c2n1] | 5-(2,8- dimethylimidazo[1,2- a]pyridin-6-yl)-7-fluoro- 2-(piperidin-4-yl)-1H- benzo[d]imidazole |
| 148 | 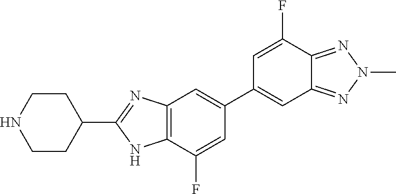 [SMILES: Cn1nc2cc(-c3cc(F)c4[nH]c(C5CCNCC5)nc4c3)cc(F)c2n1] | 4-fluoro-6-(7-fluoro-2- (piperidin-4-yl)-1H- benzo[d]imidazol-5-yl)- 2-methyl-2H- benzo[d][1,2,3]triazole |
| 149 | 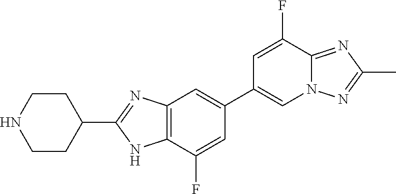 [SMILES: Cc1nc2c(F)cc(-c3cc(F)c4[nH]c(C5CCNCC5)nc4c3)cn2n1] | 8-fluoro-6-(7-fluoro-2- (piperidin-4-yl)-1H- benzo[d]imidazol-5-yl)- 2-methyl- [1,2,4]triazolo[1,5- a]pyridine |
| 417 | 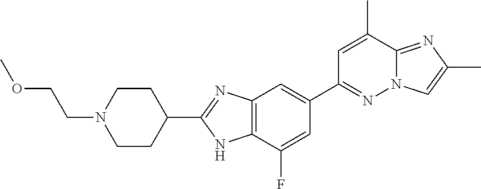 | 6-(7-fluoro-2-(1-(2- methoxyethyl)piperidin- 4-yl)-1H- benzo[d]imidazol-5-yl)- 2,8- dimethylimidazo[1,2- b]pyridazine |
| 418 | 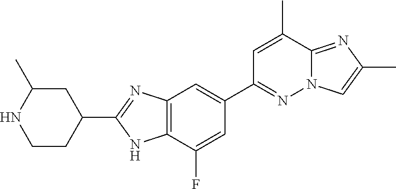 [SMILES: Cc1cn2nc(-c3cc(F)c4[nH]c(C5CCNC(C)C5)nc4c3)cc(C)c2n1] | 6-(7-fluoro-2-(2- methylpiperidin-4-yl)- 1H-benzo[d]imidazol-5- yl)-2,8- dimethylimidazo[1,2- b]pyridazine |
| 419 | 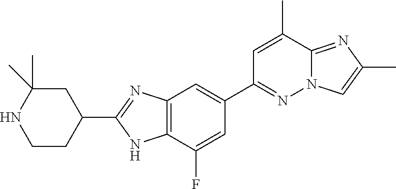 | 6-(2-(2,2- dimethylpiperidin-4-yl)- 7-fluoro-1H- benzo[d]imidazol-5-yl)- 2,8- dimethylimidazo[1,2- blpyridazine |
| 420 | 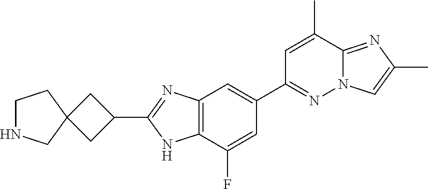 [SMILES: Cc1cn2nc(-c3cc(F)c4[nH]c(C5CC6(CCNC6)C5)nc4c3)cc(C)c2n1] | 6-(7-fluoro-2-(6- azaspiro[3.4]octan-2- yl)-1H- benzo[d]imidazol-5-yl)- 2,8- dimethylimidazo[1,2- b]pyridazine |
| 421 | 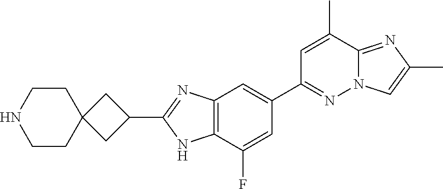 [SMILES: Cc1cn2nc(-c3cc(F)c4[nH]c(C5CC6(CCNCC6)C5)nc4c3)cc(C)c2n1] | 6-(7-fluoro-2-(7- azaspiro[3.5]nonan-2- yl)-1H- benzo[d]imidazol-5-yl)- 2,8- dimethylimidazo[1,2- b]pyridazine |
| 422 | 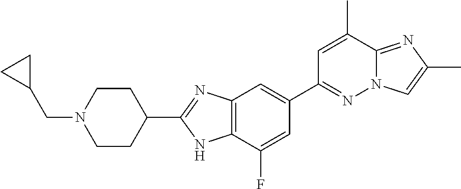 [SMILES: Cc1cn2nc(-c3cc(F)c4[nH]c(C5CCN(CC6CC6)CC5)nc4c3)cc(C)c2n1] | 6-(2-(1- (cyclopropylmethyl) piperidin-4-yl)-7- fluoro-1H- benzo[d]imidazol-5-yl)- 2,8- dimethylimidazo[1,2- b]pyridazine |
| 423 | 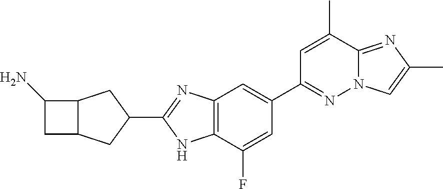 [SMILES: Cc1cn2nc(-c3cc(F)c4[nH]c(C5CC6CC(N)C6C5)nc4c3)cc(C)c2n1] | 3-(5-(2,8- dimethylimidazo[1,2- b]pyridazin-6-yl)-7- fluoro-1H- benzo[d]imidazol-2- yl)bicyclo[3.2.0]heptan- 6-amine |
| 424 | 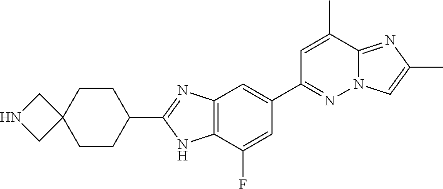 [SMILES: Cc1cn2nc(-c3cc(F)c4[nH]c(C5CCC6(CC5)CNC6)nc4c3)cc(C)c2n1] | 6-(7-fluoro-2-(2- azaspiro[3.5]nonan-7- yl)-1H- benzo[d]imidazol-5-yl)- 2,8- dimethylimidazo[1,2- b]pyridazine |
| 425 | 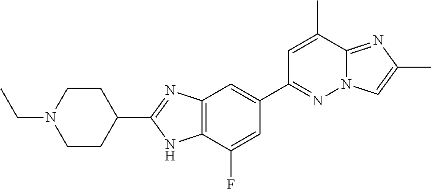 [SMILES: CCN1CCC(c2nc3cc(-c4cc(C)c5nc(C)cn5n4)cc(F)c3[nH]2)CC1] | 6-(2-(1-ethylpiperidin-4- yl)-7-fluoro-1H- benzo[d]imidazol-5-yl)- 2,8- dimethylimidazo[1,2- b]pyridazine |
| 426 | 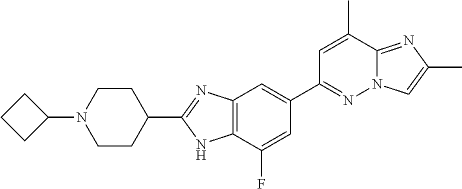 [SMILES: Cc1cn2nc(-c3cc(F)c4[nH]c(C5CCN(C6CCC6)CC5)nc4c3)cc(C)c2n1] | 6-(2-(1- cyclobutylpiperidin-4- yl)-7-fluoro-1H- benzo[d]imidazol-5-yl)- 2,8- dimethylimidazo[1,2- b]pyridazine |
| 427 | 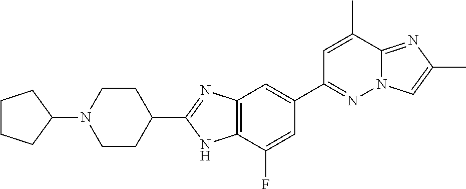 [SMILES: Cc1cn2nc(-c3cc(F)c4[nH]c(C5CCN(C6CCCC6)CC5)nc4c3)cc(C)c2n1] | 6-(2-(1- cyclopentylpiperidin-4- yl)-7-fluoro-1H- benzo[d]imidazol-5-yl)- 2,8- dimethylimidazo[1,2- b]pyridazine |
| 428 | 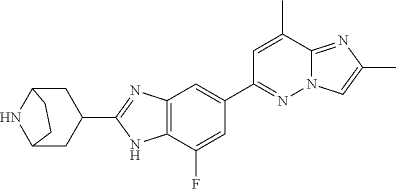 [SMILES: Cc1cn2nc(-c3cc(F)c4[nH]c(C5CC6CCC(C5)N6)nc4c3)cc(C)c2n1] | 6-(2-(8- azabicyclo[3.2.1]octan- 3-yl)-7-fluoro-1H- benzo[d]imidazol-5-yl)- 2,8- dimethylimidazo[1,2- b]pyridazine |
| 429 | 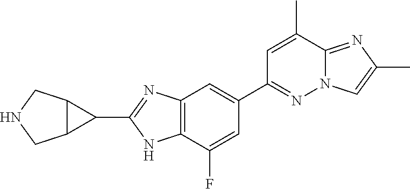 [SMILES: Cc1cn2nc(-c3cc(F)c4[nH]c(C5C6CNCC65)nc4c3)cc(C)c2n1] | 6-(2-(3- azabicyclo[3.1.0]hexan- 6-yl)-7-fluoro-1H- benzo[d]imidazol-5-yl)- 2,8- dimethylimidazo[1,2- b]pyridazine |
| 430 | 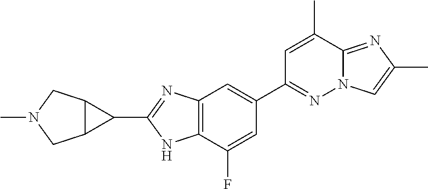 | 6-(7-fluoro-2-(3-methyl- 3- azabicyclo[3.1.0]hexan- 6-yl)-1H- benzo[d]imidazol-5-yl)- 2,8- dimethylimidazo[1,2- b]pyridazine |
| 150 | 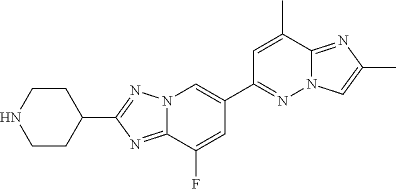 [SMILES: Cc1cn2nc(-c3cc(F)c4nc(C5CCNCC5)nn4c3)cc(C)c2n1] | 6-(2,8- dimethylimidazo[1,2- b]pyridazin-6-yl)-8- fluoro-2-(piperidin-4- yl)-[1,2,4]triazolo[1,5- a]pyridine |
| 151 | 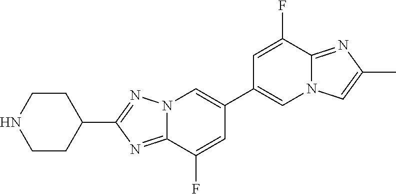 [SMILES: Cc1cn2cc(-c3cc(F)c4nc(C5CCNCC5)nn4c3)cc(F)c2n1] | 8-fluoro-6-(8-fluoro-2- methylimidazo[1,2- a]pyridin-6-yl)-2- (piperidin-4-yl)- [1,2,4]triazolo[1,5- a]pyridine |
| 152 | 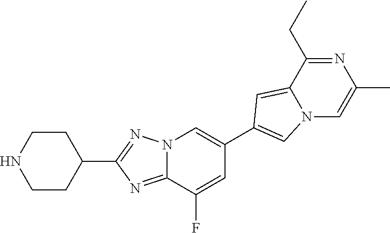 [SMILES: CCc1nc(C)cn2cc(-c3cc(F)c4nc(C5CCNCC5)nn4c3)cc12] | 6-(1,3- dimethylpyrrolo[1,2- a]pyrazin-7-yl)-8- fluoro-2-(piperidin-4- yl)-[1,2,4]triazolo[1,5- a]pyridine |
| 431 | 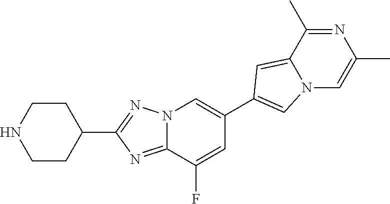 [SMILES: Cc1cn2cc(-c3cc(F)c4nc(C5CCNCC5)nn4c3)cc2c(C)n1] | 6-(1,3- dimethylpyrrolo[1,2- a]pyrazin-7-yl)-8- fluoro-2-(piperidin-4- yl)-[1,2,4]triazolo[1,5- a]pyridine |
| 432 | 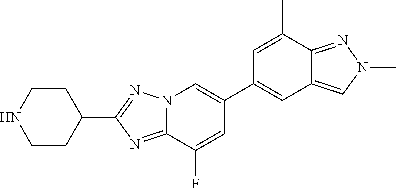 [SMILES: Cc1cc(-c2cc(F)c3nc(C4CCNCC4)nn3c2)cc2cn(C)nc12] | 6-(2,7-dimethyl-2H- indazol-5-yl)-8-fluoro- 2-(piperidin-4-yl)- [1,2,4]triazolo[1,5- a]pyridine |
| 433 | 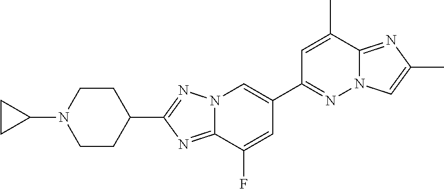 [SMILES: Cc1cn2nc(-c3cc(F)c4nc(C5CCN(C6CC6)CC5)nn4c3)cc(C)c2n1] | 2-(1- cyclopropylpiperidin-4- yl)-6-(2,8- dimethylimidazo[1,2- b]pyridazin-6-yl)-8- fluoro- [1,2,4]triazolo[1,5- a]pyridine |
| 434 | 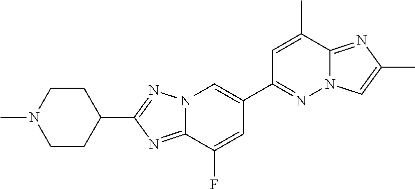 | 6-(2,8- dimethylimidazo[1,2- b]pyridazin-6-yl)-8- fluoro-2-(1- methylpiperidin-4-yl)- [1,2,4]triazolo[1,5- a]pyridine |
| 435 | 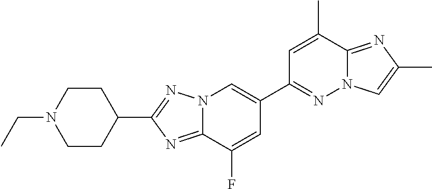 [SMILES: CCN1CCC(c2nc3c(F)cc(-c4cc(C)c5nc(C)cn5n4)cn3n2)CC1] | 6-(2,8- dimethylimidazo[1,2- b]pyridazin-6-yl)-2-(1- ethylpiperidin-4-yl)-8- fluoro- [1,2,4]triazolo[1,5- a]pyridine |
| 436 | 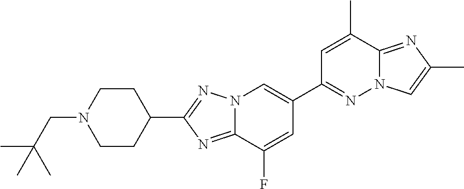 [SMILES: Cc1cn2nc(-c3cc(F)c4nc(C5CCN(CC(C)(C)C)CC5)nn4c3)cc(C)c2n1] | 6-(2,8- dimethylimidazo[1,2- b]pyridazin-6-yl)-8- fluoro-2-(1- neopentylpiperidin-4- yl)-[1,2,4]triazolo[1,5- a]pyridine |
| 437 | 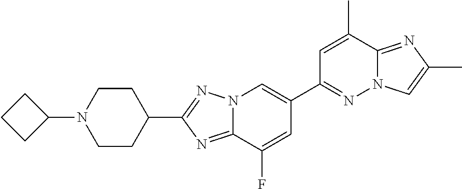 [SMILES: Cc1cn2nc(-c3cc(F)c4nc(C5CCN(C6CCC6)CC5)nn4c3)cc(C)c2n1] | 2-(1- cyclobutylpiperidin-4- yl)-6-(2,8- dimethylimidazo[1,2- b]pyridazin-6-yl)-8- fluoro- [1,2,4]triazolo[1,5- a]pyridine |
| 438 | 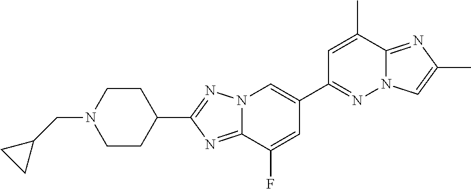 [SMILES: Cc1cn2nc(-c3cc(F)c4nc(C5CCN(CC6CC6)CC5)nn4c3)cc(C)c2n1] | 2-(1- (cyclopropylmethyl) piperidin-4-yl)-6-(2,8- dimethylimidazo[1,2- b]pyridazin-6-yl)-8- fluoro- [1,2,4]triazolo[1,5- a]pyridine |
| 439 | 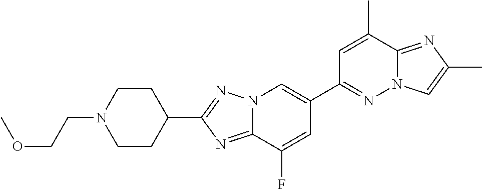 | 6-(2,8- dimethylimidazo[1,2- b]pyridazin-6-yl)-8- fluoro-2-(1-(2- methoxyethyl)piperidin- 4-yl)-[1,2,4]triazolo[1,5- a]pyridine |
| 440 | 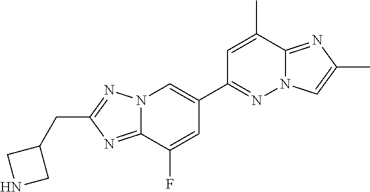 [SMILES: Cc1cn2nc(-c3cc(F)c4nc(CC5CNC5)nn4c3)cc(C)c2n1] | 2-(azetidin-3-ylmethyl)- 6-(2,8- dimethylimidazo[1,2- b]pyridazin-6-yl)-8- fluoro- [1,2,4]triazolo[1,5- a]pyridine |
| 441 | 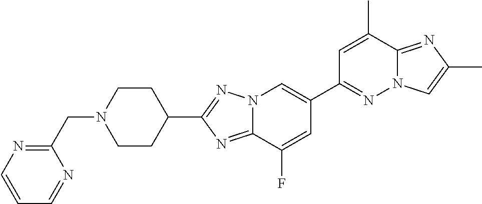 [SMILES: Cc1cn2nc(-c3cc(F)c4nc(C5CCN(Cc6ncccn6)CC5)nn4c3)cc(C)c2n1] | 6-(2,8- dimethylimidazo[1,2- b]pyridazin-6-yl)-8- fluoro-2-(1-(pyrimidin- 2-ylmethyl)piperidin-4- yl)-[1,2,4]triazolo[1,5- a]pyridine |
| 442 | 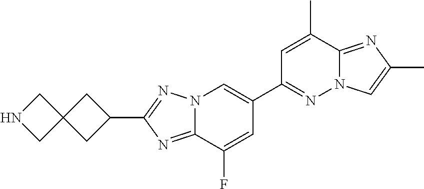 [SMILES: Cc1cn2nc(-c3cc(F)c4nc(C5CC6(CNC6)C5)nn4c3)cc(C)c2n1] | 6-(2,8- dimethylimidazo[1,2- b]pyridazin-6-yl)-8- fluoro-2-(2- azaspiro[3.3]heptan-6- yl)-[1,2,4]triazolo[1,5- a]pyridine |
| 443 | 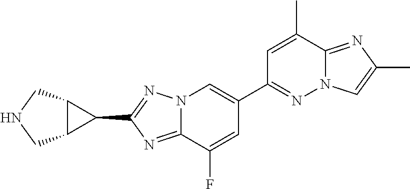 [SMILES: Cc1cn2nc(-c3cc(F)c4nc([C@H]5[C@@H]6CNC[C@@H]65)nn4c3)cc(C)c2n1] | 2-((1R,5S,6r)-3- azabicyclo[3.1.0]hexan- 6-yl)-6-(2,8- dimethylimidazo[1,2- b]pyridazin-6-yl)-8- fluoro- [1,2,4]triazolo[1,5- a]pyridine |
| 444 | 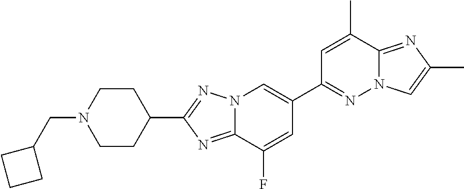 [SMILES: Cc1cn2nc(-c3cc(F)c4nc(C5CCN(CC6CCC6)CC5)nn4c3)cc(C)c2n1] | 2-(1- (cyclobutylmethyl) piperidin-4-yl)-6-(2,8- dimethylimidazo[1,2- b]pyridazin-6-yl)-8- fluoro- [1,2,4]triazolo[1,5- a]pyridine |
| 445 | 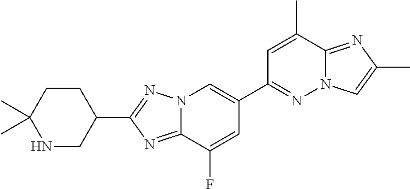 | 6-(2,8- dimethylimidazo[1,2- b]pyridazin-6-yl)-2-(6,6- dimethylpiperidin-3-yl)- 8-fluoro- [1,2,4]triazolo[1,5- a]pyridine |
| 446 | 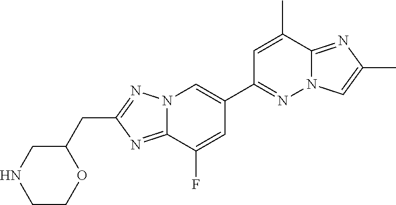 [SMILES: Cc1cn2nc(-c3cc(F)c4nc(CC5CNCCO5)nn4c3)cc(C)c2n1] | 2-((6-(2,8- dimethylimidazo[1,2- b]pyridazin-6-yl)-8- fluoro- [1,2,4]triazolo[1,5- a]pyridin-2- yl)methyl)morpholine |
| 447 | 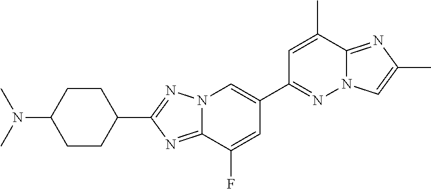 | 4-(6-(2,8- dimethylimidazo[1,2- b]pyridazin-6-yl)-8- fluoro- [1,2,4]triazolo[1,5- a]pyridin-2-yl)-N,N- dimethylcyclohexan-1- amine |
| 448 | 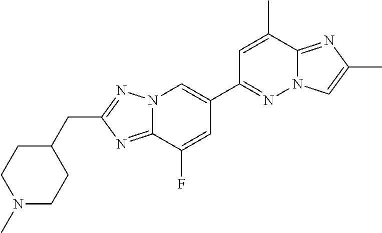 [SMILES: Cc1cn2nc(-c3cc(F)c4nc(CC5CCN(C)CC5)nn4c3)cc(C)c2n1] | 6-(2,8- dimethylimidazo[1,2- b]pyridazin-6-yl)-8- fluoro-2-((1- methylpiperidin-4- yl)methyl)- [1,2,4]triazolo[1,5- a]pyridine |
| 449 | 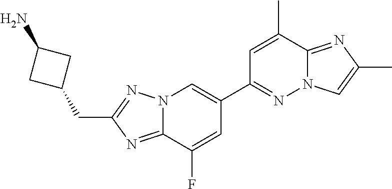 [SMILES: Cc1cn2nc(-c3cc(F)c4nc(C[C@H]5C[C@H](N)C5)nn4c3)cc(C)c2n1] | (1r,3r)-3-((6-(2,8- dimethylimidazo[1,2- b]pyridazin-6-yl)-8- fluoro- [1,2,4]triazolo[1,5- a]pyridin-2- yl)methyl)cyclobutan-1- amine |
| 450 | 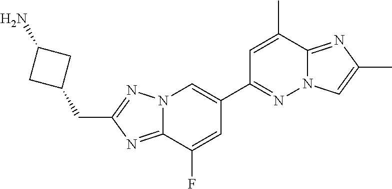 [SMILES: Cc1cn2nc(-c3cc(F)c4nc(C[C@H]5C[C@@H](N)C5)nn4c3)cc(C)c2n1] | (1s,3s)-3-((6-(2,8- dimethylimidazo[1,2- b]pyridazin-6-yl)-8- fluoro- [1,2,4]triazolo[1,5- a]pyridin-2- yl)methyl)cyclobutan-1- amine |
| 451 | 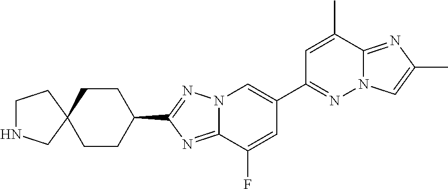 [SMILES: Cc1cn2nc(-c3cc(F)c4nc([C@H]5CC[C@]6(CCNC6)CC5)nn4c3)cc(C)c2n1] | (5s,8s)-8-(6-(2,8- dimethylimidazo[1,2- b]pyridazin-6-yl)-8- fluoro- [1,2,4]triazolo[1,5- a]pyridin-2-yl)-2- azaspiro[4.5]decane |
| 452 | 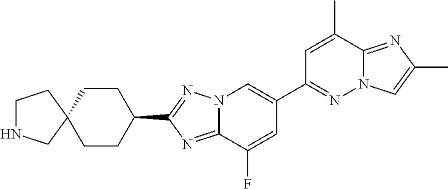 [SMILES: Cc1cn2nc(-c3cc(F)c4nc([C@H]5CC[C@@]6(CCNC6)CC5)nn4c3)cc(C)c2n1] | (5r,8r)-8-(6-(2,8- dimethylimidazo[1,2- b]pyridazin-6-yl)-8- fluoro- [1,2,4]triazolo[1,5- a]pyridin-2-yl)-2- azaspiro[4.5]decane |
| 453 | 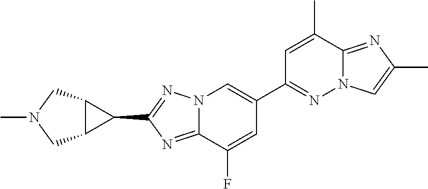 | 6-(2,8- dimethylimidazo[1,2- b]pyridazin-6-yl)-8- fluoro-2-((1R,5S,6r)-3- methyl-3- azabicyclo[3.1.0]hexan- 6-yl)-[1,2,4]triazolo[1,5- a]pyridine |
| 454 | 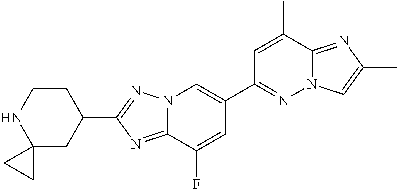 [SMILES: Cc1cn2nc(-c3cc(F)c4nc(C5CCNC6(CC6)C5)nn4c3)cc(C)c2n1] | 6-(2,8- dimethylimidazo[1,2- b]pyridazin-6-yl)-8- fluoro-2-(4- azaspiro[2.5]octan-7- yl)-[1,2,4]triazolo[1,5- a]pyridine |
| 153 | 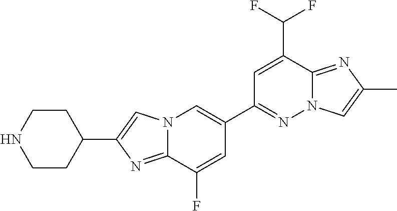 [SMILES: Cc1cn2nc(-c3cc(F)c4nc(C5CCNCC5)cn4c3)cc(C(F)F)c2n1] | 8-(difluoromethyl)-6-(8- fluoro-2-(piperidin-4- yl)imidazo[1,2- a]pyridin-6-yl)-2- methylimidazo[1,2- b]pyridazine |
| 154 | 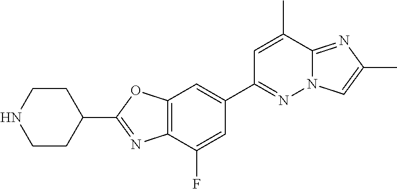 [SMILES: Cc1cn2nc(-c3cc(F)c4nc(C5CCNCC5)oc4c3)cc(C)c2n1] | 6-(2,8- dimethylimidazo[1,2- b]pyridazin-6-yl)-4- fluoro-2-(piperidin-4- yl)benzo[d]oxazole |
| 155 | 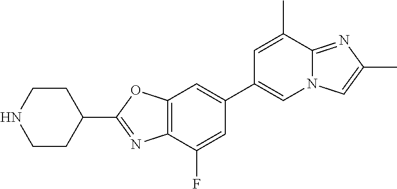 [SMILES: Cc1cn2cc(-c3cc(F)c4nc(C5CCNCC5)oc4c3)cc(C)c2n1] | 6-(2,8- dimethylimidazo[1,2- a]pyridin-6-yl)-4-fluoro- 2-(piperidin-4- yl)benzo[d]oxazole |
| 156 | 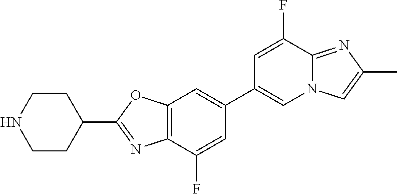 [SMILES: Cc1cn2cc(-c3cc(F)c4nc(C5CCNCC5)oc4c3)cc(F)c2n1] | 4-fluoro-6-(8-fluoro-2- methylimidazo[1,2- a]pyridin-6-yl)-2- (piperidin-4- yl)benzo[d]oxazole |
| 157 | 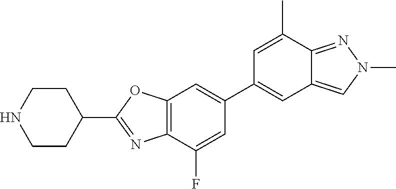 [SMILES: Cc1cc(-c2cc(F)c3nc(C4CCNCC4)oc3c2)cc2cn(C)nc12] | 6-(2,7-dimethyl-2H- indazol-5-yl)-4-fluoro- 2-(piperidin-4- yl)benzo[d]oxazole |
| 455 | 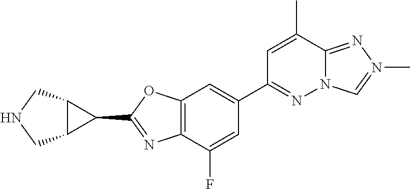 | 2-((1R,5S,6r)-3- azabicyclo[3.1.0]hexan- 6-yl)-6-(2,8- dimethylimidazo[1,2- b]pyridazin-6-yl)-4- fluorobenzo[d]oxazole |
| 456 | 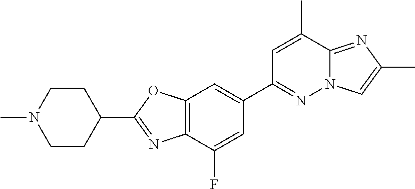 | 6-(2,8- dimethylimidazo[1,2- b]pyridazin-6-yl)-4- fluoro-2-(1- methylpiperidin-4- yl)benzo[d]oxazole |
| 457 | 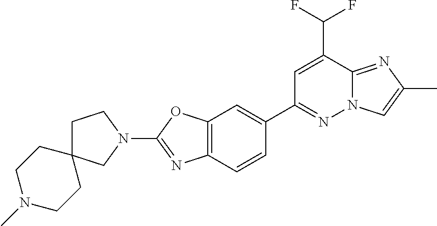 | 6-(8-(difluoromethyl)-2- methylimidazo[1,2- b]pyridazin-6-yl)-2-(8- methyl-2,8- diazaspiro[4.5]decan-2- yl)benzo[d]oxazole |
| 458 | 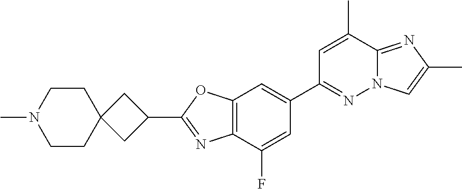 | 6-(2,8- dimethylimidazo[1,2- b]pyridazin-6-yl)-4- fluoro-2-(7-methyl-7- azaspiro[3.5]nonan-2- yl)benzo[d]oxazole |
| 459 | 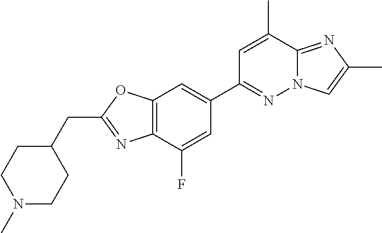 [SMILES: Cc1cn2nc(-c3cc(F)c4nc(CC5CCN(C)CC5)oc4c3)cc(C)c2n1] | 6-(2,8- dimethylimidazo[1,2- b]pyridazin-6-yl)-4- fluoro-2-((1- methylpiperidin-4- yl)methyl)benzo[d] oxazole |
| 460 | 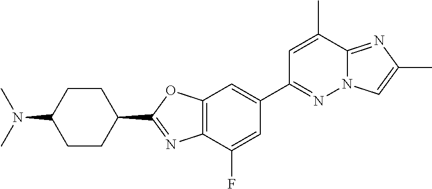 | (1s,4s)-4-(6-(2,8- dimethylimidazo[1,2- b]pyridazin-6-yl)-4- fluorobenzo[d]oxazol-2- yl)-N,N- dimethylcyclohexan-1- amine |
| 461 | 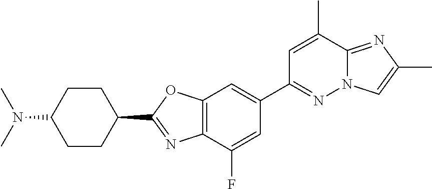 | (1r,4r)-4-(6-(2,8- dimethylimidazo[1,2- b]pyridazin-6-yl)-4- fluorobenzo[d]oxazol-2- yl)-N,N- dimethylcyclohexan-1- amine |
| 462 | 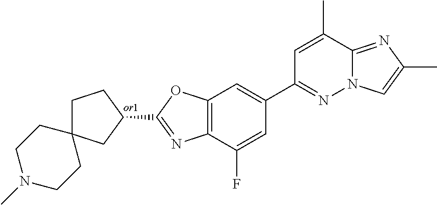 | rel-(R)-6-(2,8- dimethylimidazo[1,2- b]pyridazin-6-yl)-4- fluoro-2-(8-methyl-8- azaspiro[4.5]decan-2- yl)benzo[d]oxazole |
| 463 | 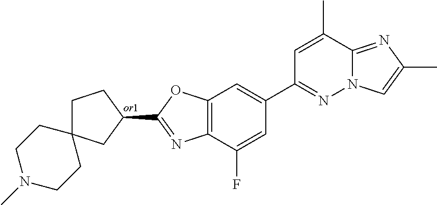 | rel-(R)-6-(2,8- dimethylimidazo[1,2- b]pyridazin-6-yl)-4- fluoro-2-(8-methyl-8- azaspiro[4.5]decan-2- yl)benzo[d]oxazole |
| 464 | 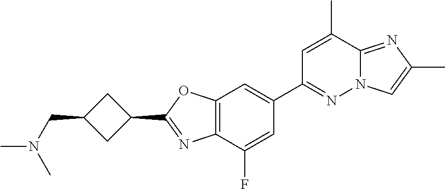 | 1-((1s,3s)-3-(6-(2,8- dimethylimidazo[1,2- b]pyridazin-6-yl)-4- fluorobenzo[d]oxazol-2- yl)cyclobutyl)-N,N- dimethylmethanamine |
| 465 | 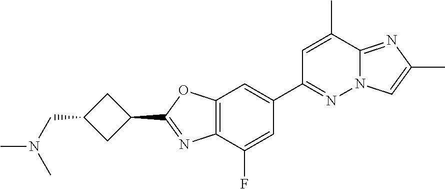 | 1-((1r,3r)-3-(6-(2,8- dimethylimidazo[1,2- b]pyridazin-6-yl)-4- fluorobenzo[d]oxazol-2- yl)cyclobutyl)-N,N- dimethylmethanamine |
| 466 | 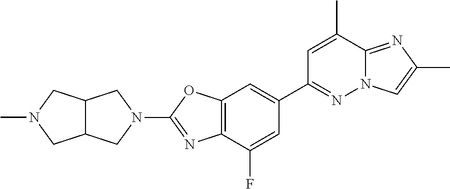 | 6-(2,8- dimethylimidazo[1,2- b]pyridazin-6-yl)-4- fluoro-2-(5- methylhexahydropyrrolo [3,4-c]pyrrol-2(1H)- yl)benzo[d]oxazole |
| 467 | 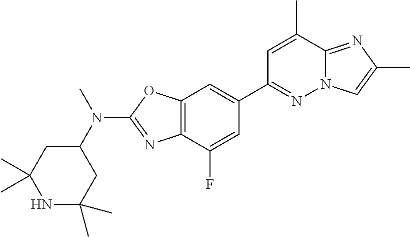 | 6-(2,8- dimethylimidazo[1,2- b]pyridazin-6-yl)-4- fluoro-N-methyl-N- (2,2,6,6- tetramethylpiperidin-4- yl)benzo[d]oxazol-2- amine |
| 468 | 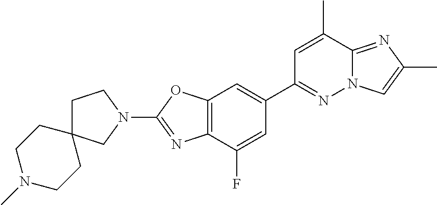 | 6-(2,8- dimethylimidazo[1,2- b]pyridazin-6-yl)-4- fluoro-2-(8-methyl-2,8- diazaspiro[4.5]decan-2- yl)benzo[d]oxazole |
| 469 | 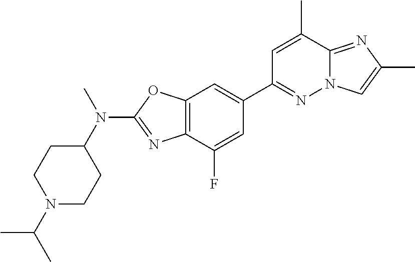 [SMILES: Cc1cn2nc(-c3cc(F)c4nc(N(C)C5CCN(C(C)C)CC5)oc4c3)cc(C)c2n1] | 6-(2,8- dimethylimidazo[1,2- b]pyridazin-6-yl)-4- fluoro-N-(1- isopropylpiperidin-4- yl)-N- methylbenzo[d]oxazol- 2-amine |
| 470 | 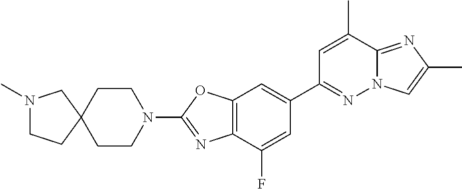 | 6-(2,8- dimethylimidazo[1,2- b]pyridazin-6-yl)-4- fluoro-2-(2-methyl-2,8- diazaspiro[4.5]decan-8- yl)benzo[d]oxazole |
| 471 | 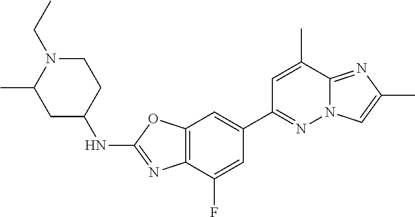 | 6-(2,8- dimethylimidazo[1,2- b]pyridazin-6-yl)-N-(1- ethyl-2-methylpiperidin- 4-yl)-4- fluorobenzo[d]oxazol-2- amine |
| 472 | 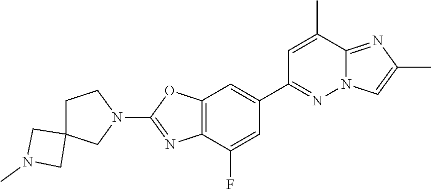 | 6-(2,8- dimethylimidazo[1,2- b]pyridazin-6-yl)-4- fluoro-2-(2-methyl-2,6- diazaspiro[3.4]octan-6- yl)benzo[d]oxazole |
| 473 | 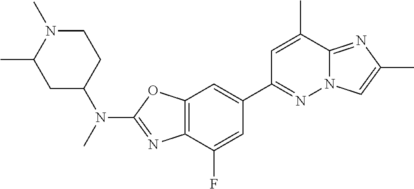 | 6-(2,8- dimethylimidazo[1,2- b]pyridazin-6-yl)-N- (1,2-dimethylpiperidin- 4-yl)-4-fluoro-N- methylbenzo[d]oxazol- 2-amine |
| 474 | 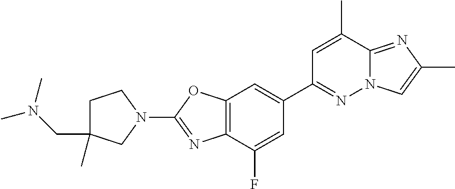 | 1-(1-(6-(2,8- dimethylimidazo[1,2- b]pyridazin-6-yl)-4- fluorobenzo[d]oxazol-2- yl)-3-methylpyrrolidin- 3-yl)-N,N- dimethylmethanamine |
| 475 | 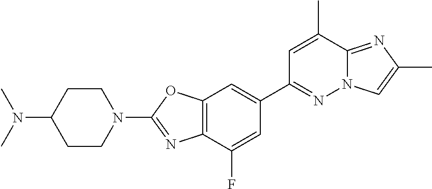 | 1-(6-(2,8- dimethylimidazo[1,2- b]pyridazin-6-yl)-4- fluorobenzo[d]oxazol-2- yl)-N,N- dimethylpiperidin-4- amine |
| 476 | 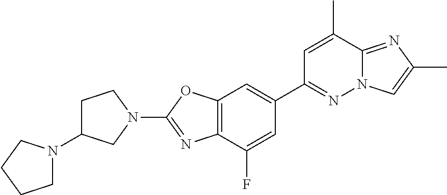 [SMILES: Cc1cn2nc(-c3cc(F)c4nc(N5CCC(N6CCCC6)C5)oc4c3)cc(C)c2n1] | 2-([1,3′-bipyrrolidin]-1′- yl)-6-(2,8- dimethylimidazo[1,2- b]pyridazin-6-yl)-4- fluorobenzo[d]oxazole |
| 477 | 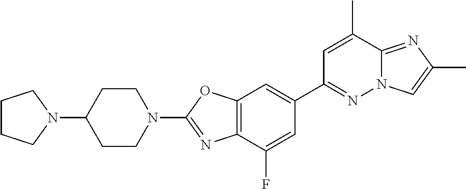 [SMILES: Cc1cn2nc(-c3cc(F)c4nc(N5CCC(N6CCCC6)CC5)oc4c3)cc(C)c2n1] | 6-(2,8- dimethylimidazo[1,2- b]pyridazin-6-yl)-4- fluoro-2-(4-(pyrrolidin- 1-yl)piperidin-1- yl)benzo[d]oxazole |
| 478 | 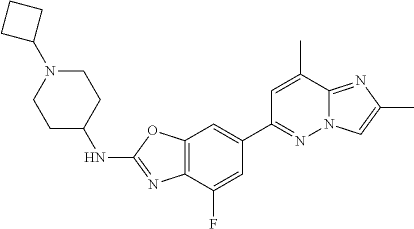 [SMILES: Cc1cn2nc(-c3cc(F)c4nc(NC5CCN(C6CCC6)CC5)oc4c3)cc(C)c2n1] | N-(1- cyclobutylpiperidin-4- yl)-6-(2,8- dimethylimidazo[1,2- b]pyridazin-6-yl)-4- fluorobenzo[d]oxazol-2- amine |
| 479 | 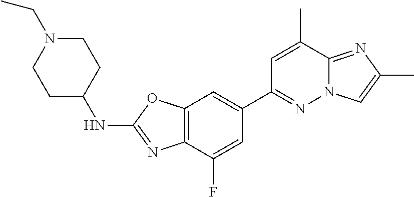 [SMILES: CCN1CCC(Nc2nc3c(F)cc(-c4cc(C)c5nc(C)cn5n4)cc3o2)CC1] | 6-(2,8- dimethylimidazo[1,2- b]pyridazin-6-yl)-N-(1- ethylpiperidin-4-yl)-4- fluorobenzo[d]oxazol-2- amine |
| 480 | 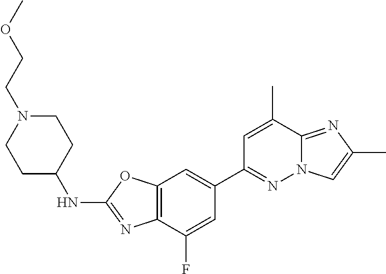 [SMILES: COCCN1CCC(Nc2nc3c(F)cc(-c4cc(C)c5nc(C)cn5n4)cc3o2)CC1] | 6-(2,8- dimethylimidazo[1,2- b]pyridazin-6-yl)-4- fluoro-N-(1-(2- methoxyethyl)piperidin- 4-yl)benzo[d]oxazol-2- amine |
| 481 | 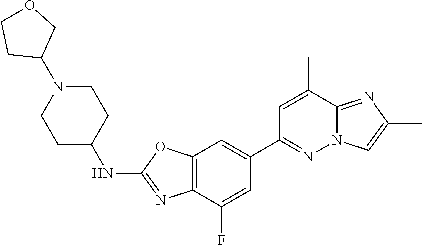 [SMILES: Cc1cn2nc(-c3cc(F)c4nc(NC5CCN(C6CCOC6)CC5)oc4c3)cc(C)c2n1] | 6-(2,8- dimethylimidazo[1,2- b]pyridazin-6-yl)-4- fluoro-N-(1- (tetrahydrofuran-3- yl)piperidin-4- yl)benzo[d]oxazol-2- amine |
| 482 | 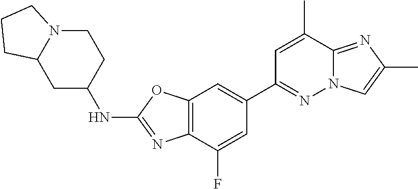 [SMILES: Cc1cn2nc(-c3cc(F)c4nc(NC5CCN6CCCC6C5)oc4c3)cc(C)c2n1] | 6-(2,8- dimethylimidazo[1,2- b]pyridazin-6-yl)-4- fluoro-N- (octahydroindolizin-7- yl)benzo[d]oxazol-2- amine |
| 483 | 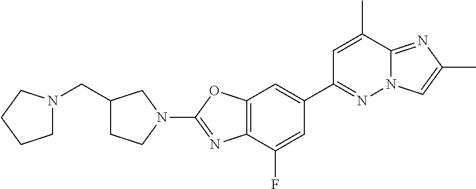 [SMILES: Cc1cn2nc(-c3cc(F)c4nc(N5CCC(CN6CCCC6)C5)oc4c3)cc(C)c2n1] | 6-(2,8- dimethylimidazo[1,2- b]pyridazin-6-yl)-4- fluoro-2-(3-(pyrrolidin- 1-ylmethyl)pyrrolidin-1- yl)benzo[d]oxazole |
| 484 | 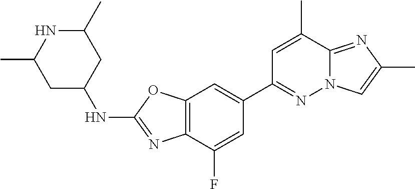 | 6-(2,8- dimethylimidazo[1,2- b]pyridazin-6-yl)-N- (2,6-dimethylpiperidin- 4-yl)-4- fluorobenzo[d]oxazol-2- amine |
| 485 | 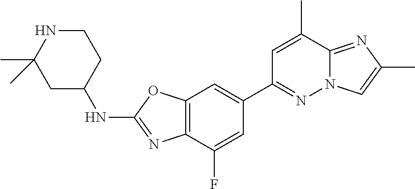 | 6-(2,8- dimethylimidazo[1,2- b]pyridazin-6-yl)-N- (2,2-dimethylpiperidin- 4-yl)-4- fluorobenzo[d]oxazol-2- amine |
| 486 | 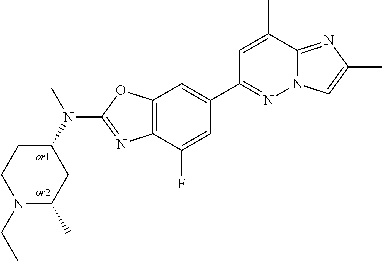 [SMILES: CCN1CC[C@H](N(C)c2nc3c(F)cc(-c4cc(C)c5nc(C)cn5n4)cc3o2)C[C@@H]1C] | rel-6-(2,8- dimethylimidazo[1,2- b]pyridazin-6-yl)-N- ((2R,4R)-1-ethyl-2- methylpiperidin-4-yl)-4- fluoro-N- methylbenzo[d]oxazol- 2-amine |
| 487 | 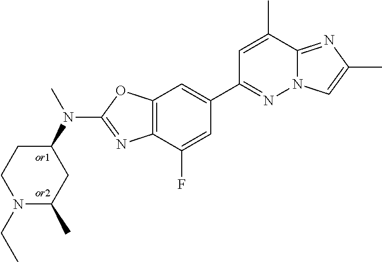 [SMILES: CCN1CC[C@@H](N(C)c2nc3c(F)cc(-c4cc(C)c5nc(C)cn5n4)cc3o2)C[C@H]1C] | rel-6-(2,8- dimethylimidazo[1,2- b]pyridazin-6-yl)-N- ((2R,4R)-1-ethyl-2- methylpiperidin-4-yl)-4- fluoro-N- methylbenzo[d]oxazol- 2-amine |
| 488 | 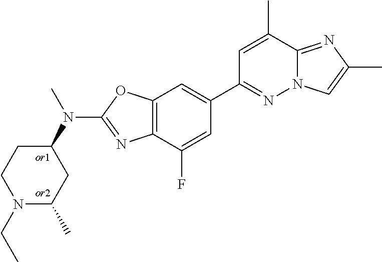 [SMILES: CCN1CC[C@@H](N(C)c2nc3c(F)cc(-c4cc(C)c5nc(C)cn5n4)cc3o2)C[C@@H]1C] | rel-6-(2,8- dimethylimidazo[1,2- b]pyridazin-6-yl)-N- ((2R,4S)-1-ethyl-2- methylpiperidin-4-yl)-4- fluoro-N- methylbenzo[d]oxazol- 2-amine |
| 489 | 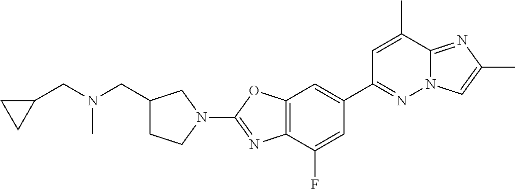 [SMILES: Cc1cn2nc(-c3cc(F)c4nc(N5CCC(CN(C)CC6CC6)C5)oc4c3)cc(C)c2n1] | 1-cyclopropyl-N-((1-(6- (2,8- dimethylimidazo[1,2- b]pyridazin-6-yl)-4- fluorobenzo[d]oxazol-2- yl)pyrrolidin-3- yl)methyl)-N- methylmethanamine |
| 490 | 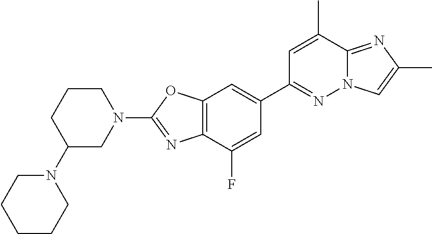 [SMILES: Cc1cn2nc(-c3cc(F)c4nc(N5CCCC(N6CCCCC6)C5)oc4c3)cc(C)c2n1] | 2-([1,3′-bipiperidin]-1′- yl)-6-(2,8- dimethylimidazo[1,2- b]pyridazin-6-yl)-4- fluorobenzo[d]oxazole |
| 491 | 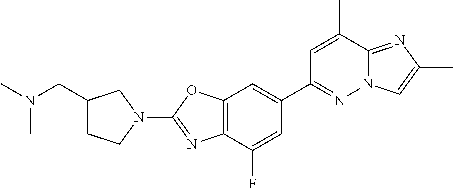 | 1-(1-(6-(2,8- dimethylimidazo[1,2- b]pyridazin-6-yl)-4- fluorobenzo[d]oxazol-2- yl)pyrrolidin-3-yl)-N,N- dimethylmethanamine |
| 492 | 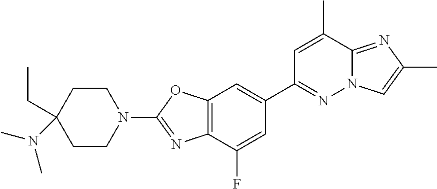 | 1-(6-(2,8- dimethylimidazo[1,2- b]pyridazin-6-yl)-4- fluorobenzo[d]oxazol-2- yl)-4-ethyl-N,N- dimethylpiperidin-4- amine |
| 493 | 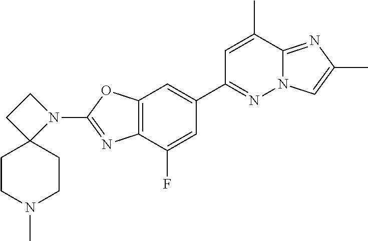 [SMILES: Cc1cn2nc(-c3cc(F)c4nc(N5CCC56CCN(C)CC6)oc4c3)cc(C)c2n1] | 6-(2,8- dimethylimidazo[1,2- b]pyridazin-6-yl)-4- fluoro-2-(7-methyl-1,7- diazaspiro[3.5]nonan-1- yl)benzo[d]oxazole |
| 494 | 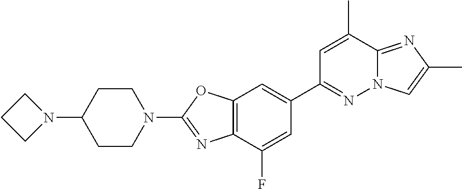 [SMILES: Cc1cn2nc(-c3cc(F)c4nc(N5CCC(N6CCC6)CC5)oc4c3)cc(C)c2n1] | 2-(4-(azetidin-1- yl)piperidin-1-yl)-6- (2,8- dimethylimidazo[1,2- b]pyridazin-6-yl)-4- fluorobenzo[d]oxazole |
| 495 | 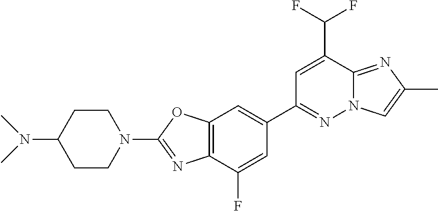 | 1-(6-(8- (difluoromethyl)-2- methylimidazo[1,2- b]pyridazin-6-yl)-4- fluorobenzo[d]oxazol-2- yl)-N,N- dimethylpiperidin-4- amine |
| 496 | 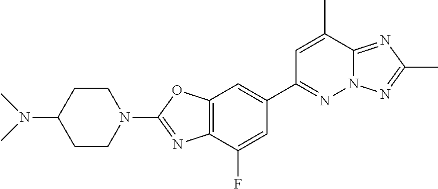 | 1-(6-(2,8-dimethyl- [1,2,4]triazolo[1,5- b]pyridazin-6-yl)-4- fluorobenzo[d]oxazol-2- yl)-N,N- dimethylpiperidin-4- amine |
| 497 | 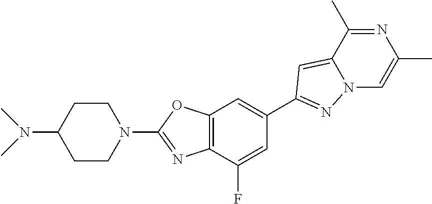 | 1-(6-(4,6- dimethylpyrazolo[1,5- a]pyrazin-2-yl)-4- fluorobenzo[d]oxazol-2- yl)-N,N- dimethylpiperidin-4- amine |
| 498 | 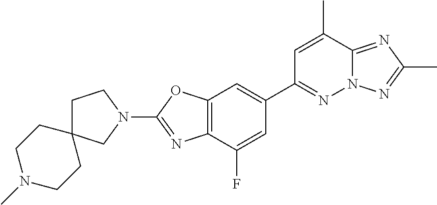 | 6-(2,8-dimethyl- [1,2,4]triazolo[1,5- b]pyridazin-6-yl)-4- fluoro-2-(8-methyl-2,8- diazaspiro[4.5]decan-2- yl)benzo[d]oxazole |
| 499 | 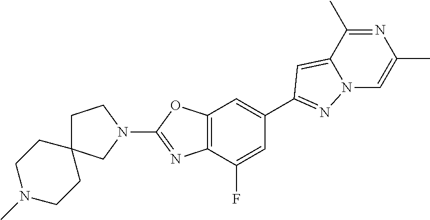 | 6-(4,6- dimethylpyrazolo[1,5- a]pyrazin-2-yl)-4- fluoro-2-(8-methyl-2,8- diazaspiro[4.5]decan-2- yl)benzo[d]oxazole |
| 500 | 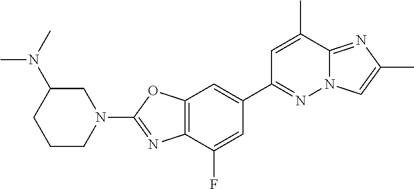 | 1-(6-(2,8- dimethylimidazo[1,2- b]pyridazin-6-yl)-4- fluorobenzo[d]oxazol-2- yl)-N,N- dimethylpiperidin-3- amine |
| 501 | 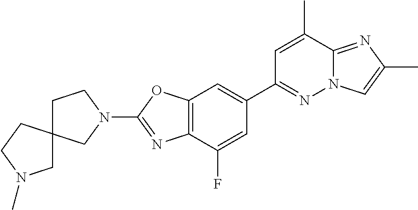 [SMILES: Cc1cn2nc(-c3cc(F)c4nc(N5CCC6(CCN(C)C6)C5)oc4c3)cc(C)c2n1] | 6-(2,8- dimethylimidazo[1,2- b]pyridazin-6-yl)-4- fluoro-2-(7-methyl-2,7- diazaspiro[4.4]nonan-2- yl)benzo[d]oxazole |
| 502 | 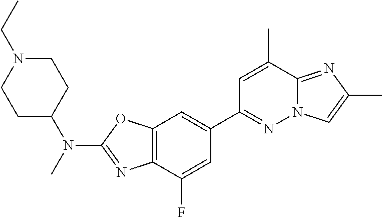 [SMILES: CCN1CCC(N(C)c2nc3c(F)cc(-c4cc(C)c5nc(C)cn5n4)cc3o2)CC1] | 6-(2,8- dimethylimidazo[1,2- b]pyridazin-6-yl)-N-(1- ethylpiperidin-4-yl)-4- fluoro-N- methylbenzo[d]oxazol- 2-amine |
| 503 | 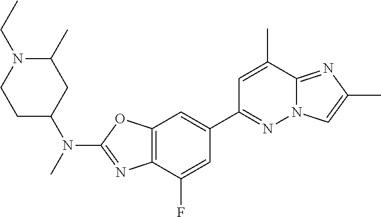 [SMILES: CCN1CCC(N(C)c2nc3c(F)cc(-c4cc(C)c5nc(C)cn5n4)cc3o2)CC1C] | 6-(2,8- dimethylimidazo[1,2- b]pyridazin-6-yl)-N-(1- ethyl-2-methylpiperidin- 4-yl)-4-fluoro-N- methylbenzo[d]oxazol- 2-amine |
| 504 | 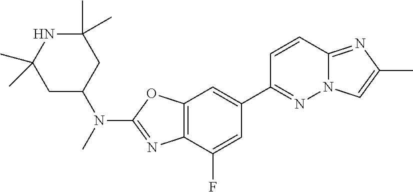 | 4-fluoro-N-methyl-6-(2- methylimidazo[1,2- b]pyridazin-6-yl)-N- (2,2,6,6- tetramethylpiperidin-4- yl)benzo[d]oxazol-2- amine |
| 505 | 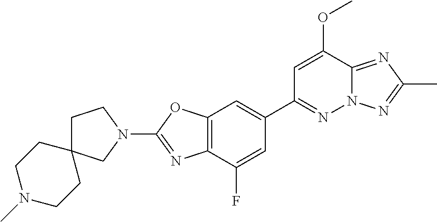 | 4-fluoro-6-(8-methoxy- 2-methyl- [1,2,4]triazolo[1,5- b]pyridazin-6-yl)-2-(8- methyl-2,8- diazaspiro[4.5]decan-2- yl)benzo[d]oxazole |
| 506 | 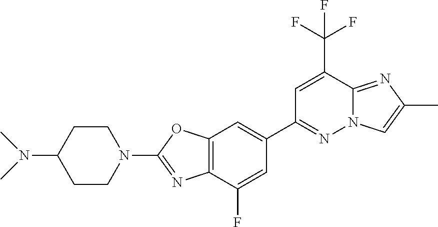 | 1-(4-fluoro-6-(2-methyl- 8- (trifluoromethyl)imidazo [1,2-b]pyridazin-6- yl)benzo[d]oxazol-2-yl)- N,N-dimethylpiperidin- 4-amine |
| 507 | 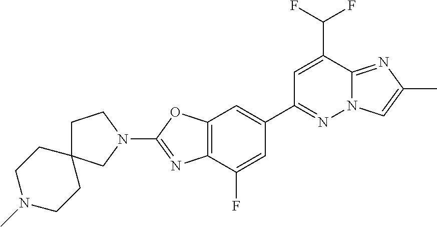 | 6-(8-(difluoromethyl)-2- methylimidazo[1,2- b]pyridazin-6-yl)-4- fluoro-2-(8-methyl-2,8- diazaspiro[4.5]decan-2- yl)benzo[d]oxazole |
| 508 | 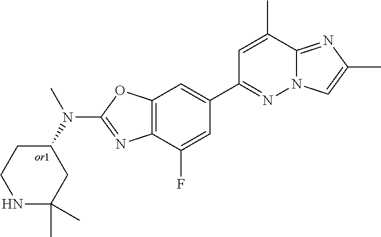 [SMILES: Cc1cn2nc(-c3cc(F)c4nc(N(C)[C@H]5CCNC(C)(C)C5)oc4c3)cc(C)c2n1] | rel-(R)-6-(2,8- dimethylimidazo[1,2- b]pyridazin-6-yl)-N- (2,2-dimethylpiperidin- 4-yl)-4-fluoro-N- methylbenzo[d]oxazol- 2-amine |
| 509 | 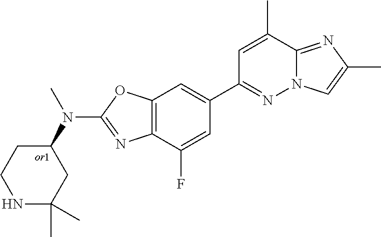 [SMILES: Cc1cn2nc(-c3cc(F)c4nc(N(C)[C@@H]5CCNC(C)(C)C5)oc4c3)cc(C)c2n1] | rel-(R)-6-(2,8- dimethylimidazo[1,2- b]pyridazin-6-yl)-N- (2,2-dimethylpiperidin- 4-yl)-4-fluoro-N- methylbenzo[d]oxazol- 2-amine |
| 510 | 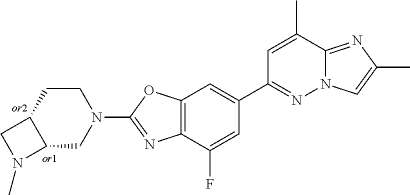 [SMILES: Cc1cn2nc(-c3cc(F)c4nc(N5CC[C@H]6CN(C)[C@H]6C5)oc4c3)cc(C)c2n1] | rel-6-(2,8- dimethylimidazo[1,2- b]pyridazin-6-yl)-4- fluoro-2-((1R,6S)-8- methyl-3,8- diazabicyclo[4.2.0]octan- 3-yl)benzo[d]oxazole |
| 511 | 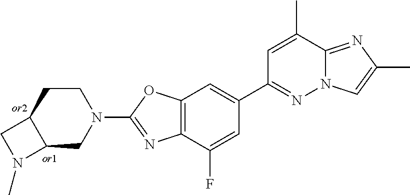 [SMILES: Cc1cn2nc(-c3cc(F)c4nc(N5CC[C@@H]6CN(C)[C@@H]6C5)oc4c3)cc(C)c2n1] | rel-6-(2,8- dimethylimidazo[1,2- b]pyridazin-6-yl)-4- fluoro-2-((1R,6S)-8- methyl-3,8- diazabicyclo[4.2.0]octan- 3-yl)benzo[d]oxazole |
| 512 | 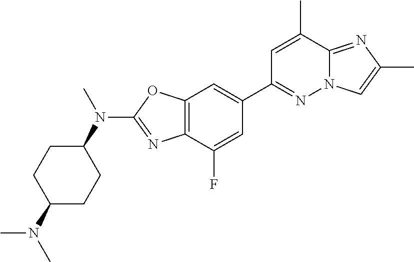 | (1s,4s)-N1-(6-(2,8- dimethylimidazo[1,2- b]pyridazin-6-yl)-4- fluorobenzo[d]oxazol-2- yl)-N1,N4,N4- trimethylcyclohexane- 1,4-diamine |
| 513 | 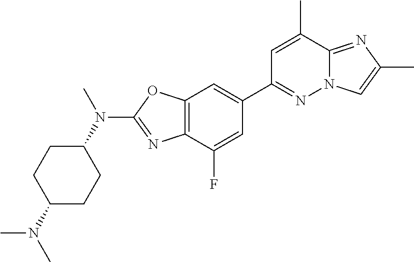 | (1s,4s)-N1-(6-(2,8- dimethylimidazo[1,2- b]pyridazin-6-yl)-4- fluorobenzo[d]oxazol-2- yl)-N1,N4,N4- trimethylcyclohexane- 1,4-diamine |
| 514 | 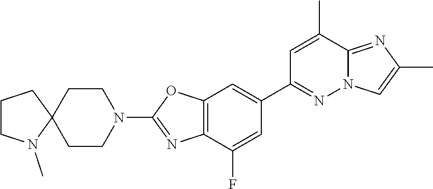 [SMILES: Cc1cn2nc(-c3cc(F)c4nc(N5CCC6(CCCN6C)CC5)oc4c3)cc(C)c2n1] | 6-(2,8- dimethylimidazo[1,2- b]pyridazin-6-yl)-4- fluoro-2-(1-methyl-1,8- diazaspiro[4.5]decan-8- yl)benzo[d]oxazole |
| 515 | 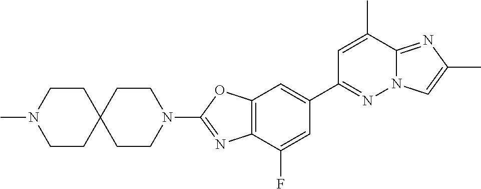 | 6-(2,8- dimethylimidazo[1,2- b]pyridazin-6-yl)-4- fluoro-2-(9-methyl-3,9- diazaspiro[5.5]undecan- 3-yl)benzo[d]oxazole |
| 516 | 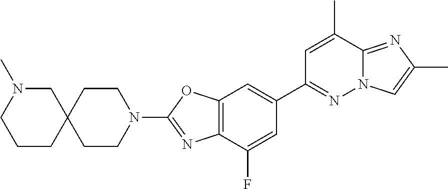 [SMILES: Cc1cn2nc(-c3cc(F)c4nc(N5CCC6(CCCN(C)C6)CC5)oc4c3)cc(C)c2n1] | 6-(2,8- dimethylimidazo[1,2- b]pyridazin-6-yl)-4- fluoro-2-(2-methyl-2,9- diazaspiro[5.5]undecan- 9-yl)benzo[d]oxazole |
| 517 | 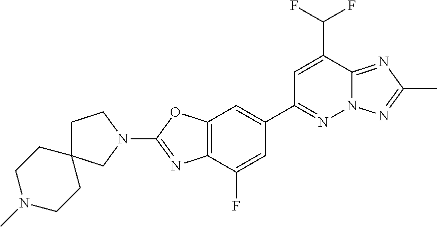 | 6-(8-(difluoromethyl)-2- methyl- [1,2,4]triazolo[1,5- b]pyridazin-6-yl)-4- fluoro-2-(8-methyl-2,8- diazaspiro[4.5]decan-2- yl)benzo[d]oxazole |
| 518 | 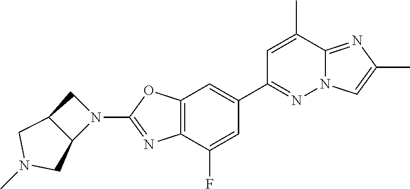 | 6-(2,8- dimethylimidazo[1,2- b]pyridazin-6-yl)-4- fluoro-2-((1S,5R)-3- methyl-3,6- diazabicyclo[3.2.0] heptan-6-yl)benzo[d] oxazole |
| 519 | 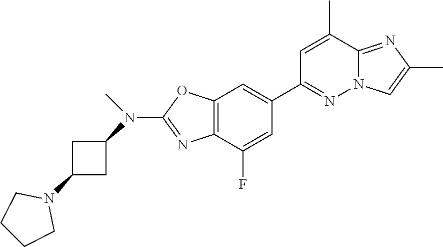 [SMILES: Cc1cn2nc(-c3cc(F)c4nc(N(C)[C@H]5C[C@@H](N6CCCC6)C5)oc4c3)cc(C)c2n1] | 6-(2,8- dimethylimidazo[1,2- b]pyridazin-6-yl)-4- fluoro-N-methyl-N- ((1s,3s)-3-(pyrrolidin-1- yl)cyclobutyl)benzo[d]o xazol-2-amine |
| 520 | 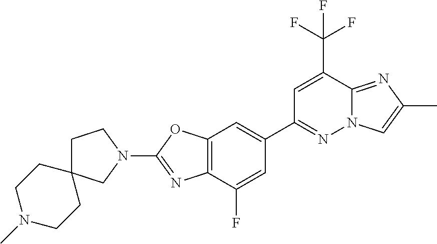 | 4-fluoro-6-(2-methyl-8- (trifluoromethyl)imidazo [1,2-b]pyridazin-6-yl)- 2-(8-methyl-2,8- diazaspiro[4.5]decan-2- yl)benzo[d]oxazole |
| 521 | 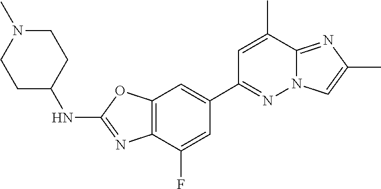 [SMILES: Cc1cn2nc(-c3cc(F)c4nc(NC5CCN(C)CC5)oc4c3)cc(C)c2n1] | 6-(2,8- dimethylimidazo[1,2- b]pyridazin-6-yl)-4- fluoro-N-(1- methylpiperidin-4- yl)benzo[d]oxazol-2- amine |
| 522 | 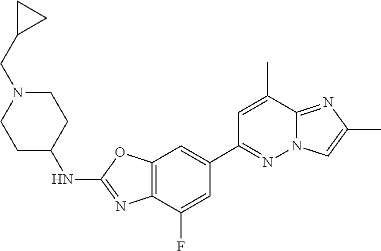 [SMILES: Cc1cn2nc(-c3cc(F)c4nc(NC5CCN(CC6CC6)CC5)oc4c3)cc(C)c2n1] | N-(1- (cyclopropylmethyl)pipe ridin-4-yl)-6-(2,8- dimethylimidazo[1,2- b]pyridazin-6-yl)-4- fluorobenzo[d]oxazol-2- amine |
| 523 | 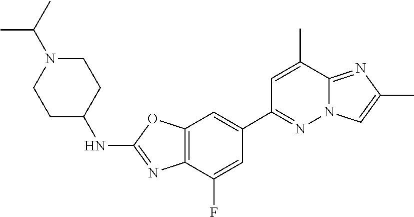 [SMILES: Cc1cn2nc(-c3cc(F)c4nc(NC5CCN(C(C)C)CC5)oc4c3)cc(C)c2n1] | 6-(2,8- dimethylimidazo[1,2- b]pyridazin-6-yl)-4- fluoro-N-(1- isopropylpiperidin-4- yl)benzo[d]oxazol-2- amine |
| 524 | 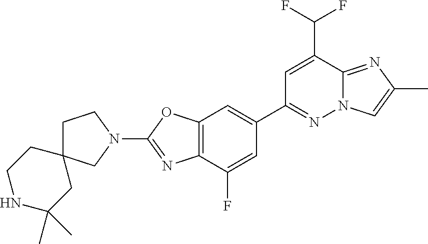 [SMILES: Cc1cn2nc(-c3cc(F)c4nc(N5CCC6(CCNC(C)(C)C6)C5)oc4c3)cc(C(F)F)c2n1] | 6-(8-(difluoromethyl)-2- methylimidazo[1,2- b]pyridazin-6-yl)-2-(7,7- dimethyl-2,8- diazaspiro[4.5]decan-2- yl)-4- fluorobenzo[d]oxazole |
| 525 | 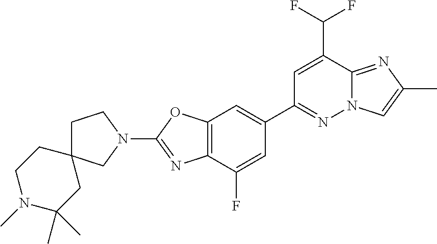 | 6-(8-(difluoromethyl)-2- methylimidazo[1,2- b]pyridazin-6-yl)-4- fluoro-2-(7,7,8- trimethyl-2,8- diazaspiro[4.5]decan-2- yl)benzo[d]oxazole |
| 526 | 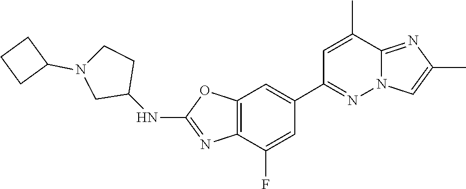 [SMILES: Cc1cn2nc(-c3cc(F)c4nc(NC5CCN(C6CCC6)C5)oc4c3)cc(C)c2n1] | N-(1- cyclobutylpyrrolidin-3- yl)-6-(2,8- dimethylimidazo[1,2- b]pyridazin-6-yl)-4- fluorobenzo[d]oxazol-2- amine |
| 527 | 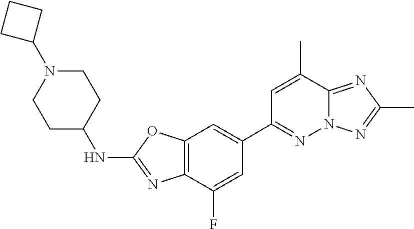 [SMILES: Cc1nc2c(C)cc(-c3cc(F)c4nc(NC5CCN(C6CCC6)CC5)oc4c3)nn2n1] | N-(1- cyclobutylpiperidin-4- yl)-6-(2,8-dimethyl- [1,2,4]triazolo[1,5- b]pyridazin-6-yl)-4- fluorobenzo[d]oxazol-2- amine |
| 528 | 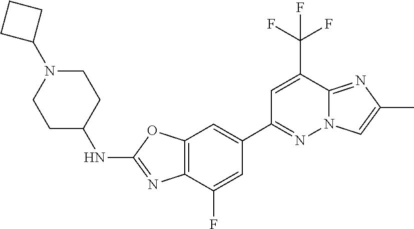 [SMILES: Cc1cn2nc(-c3cc(F)c4nc(NC5CCN(C6CCC6)CC5)oc4c3)cc(C(F)(F)F)c2n1] | N-(1- cyclobutylpiperidin-4- yl)-4-fluoro-6-(2- methyl-8- (trifluoromethyl)imidazo [1,2-b]pyridazin-6- yl)benzo[d]oxazol-2- amine |
| 529 | 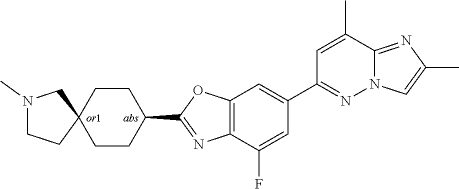 | 6-(2,8- dimethylimidazo[1,2- b]pyridazin-6-yl)-4- fluoro-2-((5S*,8r)-2- methyl-2- azaspiro[4.5]decan-8- yl)benzo[d]oxazole |
| 530 | 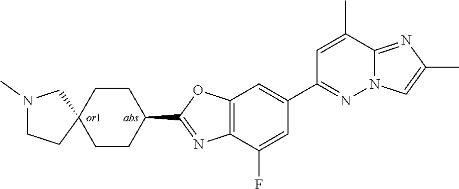 | 6-(2,8- dimethylimidazo[1,2- b]pyridazin-6-yl)-4- fluoro-2-((5S*,8r)-2- methyl-2- azaspiro[4.5]decan-8- yl)benzo[d]oxazole |
| 531 | 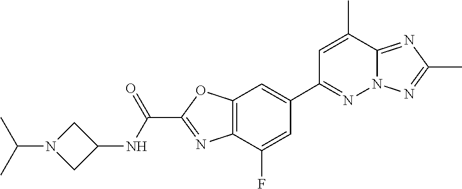 [SMILES: Cc1nc2c(C)cc(-c3cc(F)c4nc(C(=O)NC5CN(C(C)C)C5)oc4c3)nn2n1] | 6-(2,8- dimethylimidazo[1,2- b]pyridazin-6-yl)-4- fluoro-N-(1- isopropylazetidin-3- yl)benzo[d]oxazole-2- carboxamide |
| 532 | 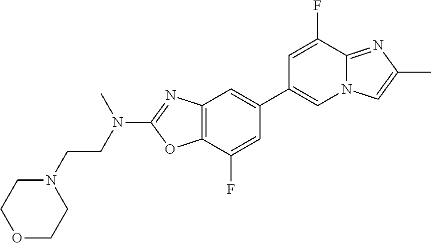 [SMILES: Cc1cn2cc(-c3cc(F)c4oc(N(C)CCN5CCOCC5)nc4c3)cc(F)c2n1] | 7-fluoro-5-(8-fluoro-2- methylimidazo[1,2- a]pyridin-6-yl)-N- methyl-N-(2- morpholinoethyl)benzo [d]oxazol-2-amine |
| 533 | 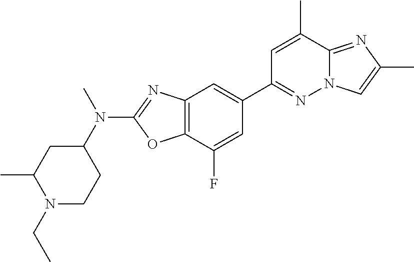 | 5-(2,8- dimethylimidazo[1,2- b]pyridazin-6-yl)-N-(1- ethyl-2-methylpiperidin- 4-yl)-7-fluoro-N- methylbenzo[d]oxazol- 2-amine |
| 534 | 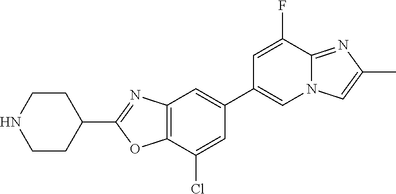 [SMILES: Cc1cn2cc(-c3cc(Cl)c4oc(C5CCNCC5)nc4c3)cc(F)c2n1] | 7-chloro-5-(8-fluoro-2- methylimidazo[1,2- a]pyridin-6-yl)-2- (piperidin-4- yl)benzo[d]oxazole |
| 535 | 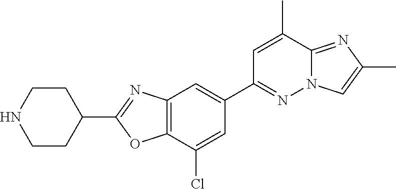 [SMILES: Cc1cn2nc(-c3cc(Cl)c4oc(C5CCNCC5)nc4c3)cc(C)c2n1] | 7-chloro-5-(2,8- dimethylimidazo[1,2- b]pyridazin-6-yl)-2- (piperidin-4- yl)benzo[d]oxazole |
| 536 | 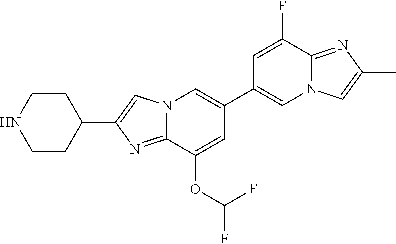 [SMILES: Cc1cn2cc(-c3cc(OC(F)F)c4nc(C5CCNCC5)cn4c3)cc(F)c2n1] | 8-(difluoromethoxy)-8′- fluoro-2′-methyl-2- (piperidin-4-yl)-6,6′- biimidazo[1,2- a]pyridine |
| 537 | 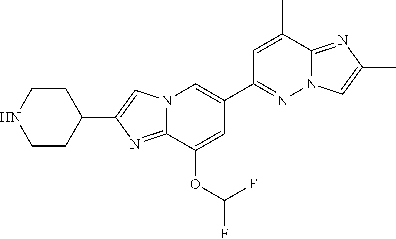 [SMILES: Cc1cn2nc(-c3cc(OC(F)F)c4nc(C5CCNCC5)cn4c3)cc(C)c2n1] | 6-(8-(difluoromethoxy)- 2-(piperidin-4- yl)imidazo[1,2- a]pyridin-6-yl)-2,8- dimethylimidazo[1,2- b]pyridazine |
| 538 | 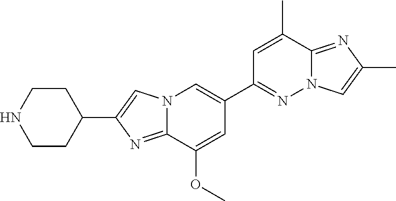 [SMILES: COc1cc(-c2cc(C)c3nc(C)cn3n2)cn2cc(C3CCNCC3)nc12] | 6-(8-methoxy-2- (piperidin-4- yl)imidazo[1,2- a]pyridin-6-yl)-2,8- dimethylimidazo[1,2- b]pyridazine |
| 539 | 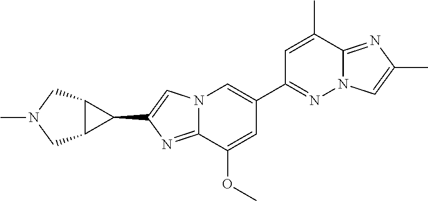 | 6-(8-methoxy-2- ((1R,5S,6r)-3-methyl-3- azabicyclo[3.1.0]hexan- 6-yl)imidazo[1,2- a]pyridin-6-yl)-2,8- dimethylimidazo[1,2- b]pyridazine |
| 540 | 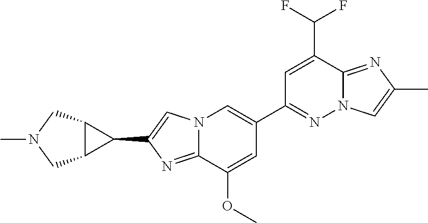 | 8-(difluoromethyl)-6-(8- methoxy-2-((1R,5S,6r)- 3-methyl-3- azabicyclo[3.1.0]hexan- 6-yl)imidazo[1,2- a]pyridin-6-yl)-2- methylimidazo[1,2- blpyridazine |
| 541 | 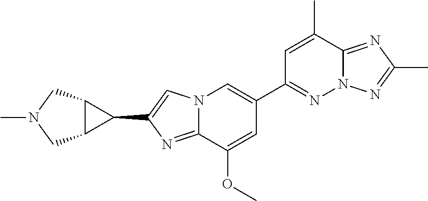 | 6-(8-methoxy-2- ((1R,5S,6r)-3-methyl-3- azabicyclo[3.1.0]hexan- 6-yl)imidazo[1,2- a]pyridin-6-yl)-2,8- dimethyl- [1,2,4]triazolo[1,5- b]pyridazine |
| 542 | 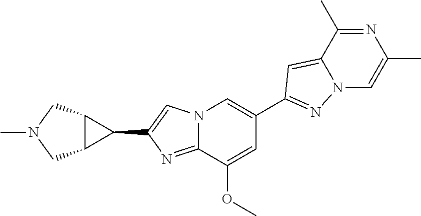 | 2-(8-methoxy-2- ((1R,5S,6r)-3-methyl-3- azabicyclo[3.1.0]hexan- 6-yl)imidazo[1,2- a]pyridin-6-yl)-4,6- dimethylpyrazolo[1,5- a]pyrazine |
| 543 | 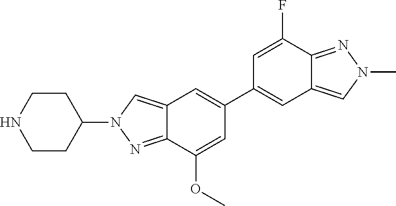 [SMILES: COc1cc(-c2cc(F)c3nn(C)cc3c2)cc2cn(C3CCNCC3)nc12] | 7-fluoro-7′-methoxy-2- methyl-2′-(piperidin-4- yl)-2H,2′H-5,5′- biindazole |
| 544 | 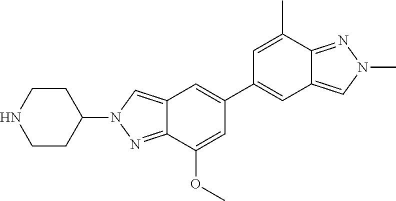 [SMILES: COc1cc(-c2cc(C)c3nn(C)cc3c2)cc2cn(C3CCNCC3)nc12] | 7-methoxy-2′,7′- dimethyl-2-(piperidin-4- yl)-2H,2′H-5,5′- biindazole |
| 545 | 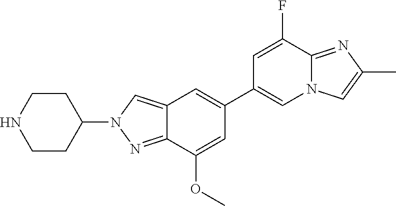 [SMILES: COc1cc(-c2cc(F)c3nc(C)cn3c2)cc2cn(C3CCNCC3)nc12] | 5-(8-fluoro-2- methylimidazo[1,2- a]pyridin-6-yl)-7- methoxy-2-(piperidin-4- yl)-2H-indazole |
| 546 | 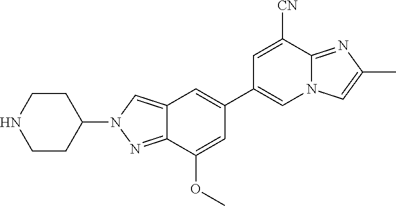 [SMILES: COc1cc(-c2cc(C#N)c3nc(C)cn3c2)cc2cn(C3CCNCC3)nc12] | 6-(7-methoxy-2- (piperidin-4-yl)-2H- indazol-5-yl)-2- methylimidazo[1,2- a]pyridine-8-carbonitrile |
| 547 | 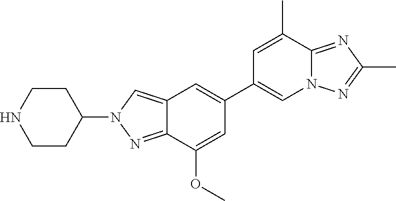 [SMILES: COc1cc(-c2cc(C)c3nc(C)nn3c2)cc2cn(C3CCNCC3)nc12] | 6-(7-methoxy-2- (piperidin-4-yl)-2H- indazol-5-yl)-2,8- dimethyl- [1,2,4]triazolo[1,5- a]pyridine |
| 548 | 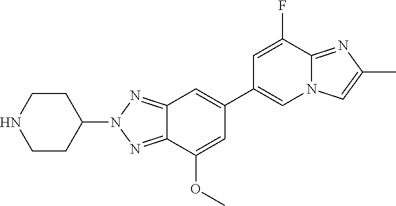 [SMILES: COc1cc(-c2cc(F)c3nc(C)cn3c2)cc2nn(C3CCNCC3)nc12] | 6-(8-fluoro-2- methylimidazo[1,2- a]pyridin-6-yl)-4- methoxy-2-(piperidin-4- yl)-2H- benzo[d][1,2,3]triazole |
| 549 | 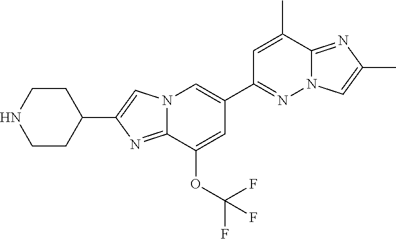 [SMILES: Cc1cn2nc(-c3cc(OC(F)(F)F)c4nc(C5CCNCC5)cn4c3)cc(C)c2n1] | 2,8-dimethyl-6-(2- (piperidin-4-yl)-8- (trifluoromethoxy) imidazo[1,2-a]pyridin- 6-yl)imidazo[1,2- b]pyridazine |
| 550 | 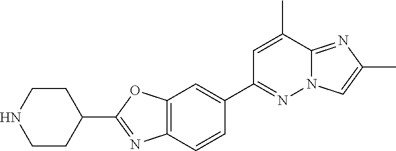 [SMILES: Cc1cn2nc(-c3ccc4nc(C5CCNCC5)oc4c3)cc(C)c2n1] | 6-(2,8- dimethylimidazo[1,2- b]pyridazin-6-yl)-2- (piperidin-4- yl)benzo[d]oxazole |
| 551 | 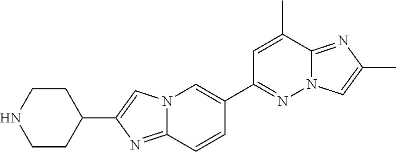 [SMILES: Cc1cn2nc(-c3ccc4nc(C5CCNCC5)cn4c3)cc(C)c2n1] | 2,8-dimethyl-6-(2- (piperidin-4- yl)imidazo[1,2- a]pyridin-6- yl)imidazo[1,2- b]pyridazine |
| 552 | 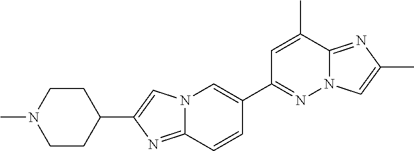 | 2,8-dimethyl-6-(2-(1- methylpiperidin-4- yl)imidazo[1,2- a]pyridin-6- yl)imidazo[1,2- blpyridazine |
| 553 | 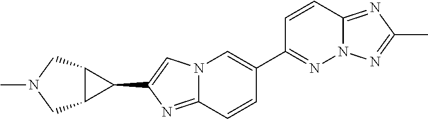 | 2-methyl-6-(2- ((1R,5S,6r)-3-methyl-3- azabicyclo[3.1.0]hexan- 6-yl)imidazo[1,2- a]pyridin-6-yl)- [1,2,4]triazolo[1,5- b]pyridazine |
| 554 | 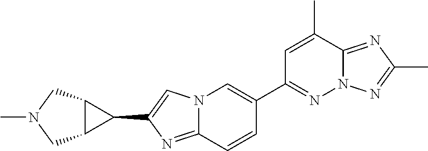 | 2,8-dimethyl-6-(2- ((1R,5S,6r)-3-methyl-3- azabicyclo[3.1.0]hexan- 6-yl)imidazo[1,2- a]pyridin-6- yl)imidazo[1,2- b]pyridazine |
| 555 | 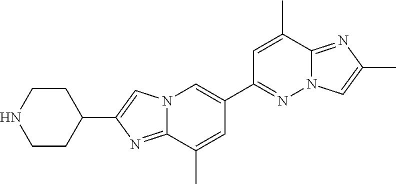 [SMILES: Cc1cn2nc(-c3cc(C)c4nc(C5CCNCC5)cn4c3)cc(C)c2n1] | 2,8-dimethyl-6-(8- methyl-2-(piperidin-4- yl)imidazo[1,2- a]pyridin-6- yl)imidazo[1,2- b]pyridazine |
| 556 | 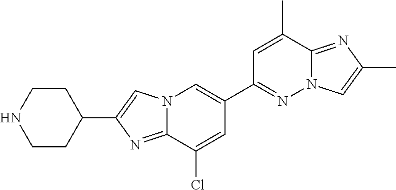 [SMILES: Cc1cn2nc(-c3cc(Cl)c4nc(C5CCNCC5)cn4c3)cc(C)c2n1] | 6-(8-chloro-2- (piperidin-4- yl)imidazo[1,2- a]pyridin-6-yl)-2,8- dimethylimidazo[1,2- b]pyridazine |
| 557 | 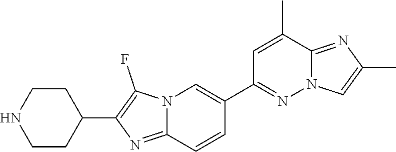 [SMILES: Cc1cn2nc(-c3ccc4nc(C5CCNCC5)c(F)n4c3)cc(C)c2n1] | 6-(3-fluoro-2-(piperidin- 4-yl)imidazo[1,2- a]pyridin-6-yl)-2,8- dimethylimidazo[1,2- blpyridazine |
| 558 | 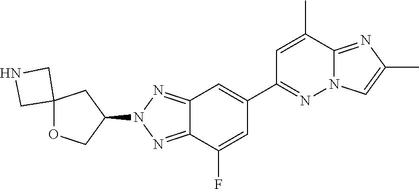 [SMILES: Cc1cn2nc(-c3cc(F)c4nn([C@H]5COC6(CNC6)C5)nc4c3)cc(C)c2n1] | (7R)-7-[6-(2,8- dimethylimidazo[1,2- b]pyridazin-6-yl)-4- fluoro-benzotriazol-2- yl]-5-oxa-2- azaspiro[3.4]octane |
| 559 | 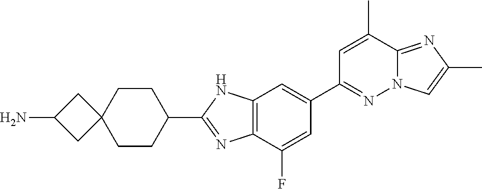 [SMILES: Cc1cn2nc(-c3cc(F)c4nc(C5CCC6(CC5)CC(N)C6)[nH]c4c3)cc(C)c2n1] | 7-(6-(2,8- dimethylimidazo[1,2- b]pyridazin-6-yl)-4- fluoro-1H- benzo[d]imidazol-2- yl)spiro[3.5]nonan-2- amine |
| 560 | 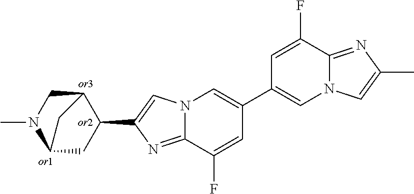 | rel-8,8′-difluoro-2- methyl-2′-((1R,4R,5S)- 2-methyl-2- azabicyclo[2.2.1]heptan- 5-yl)-6,6′-biimidazo[1,2- a]pyridine |
| 561 | 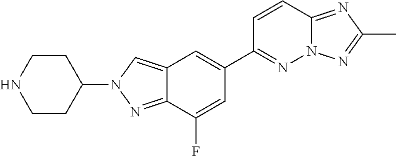 [SMILES: Cc1nc2ccc(-c3cc(F)c4nn(C5CCNCC5)cc4c3)nn2n1] | 6-(7-fluoro-2-(piperidin- 4-yl)-2H-indazol-5-yl)- 2-methyl- [1,2,4]triazolo[1,5- b]pyridazine |
| 562 | 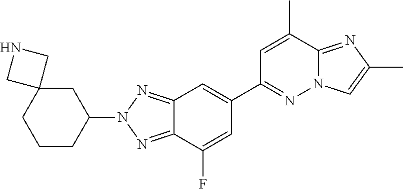 [SMILES: Cc1cn2nc(-c3cc(F)c4nn(C5CCCC6(CNC6)C5)nc4c3)cc(C)c2n1] | 6-(2,8- dimethylimidazo[1,2- b]pyridazin-6-yl)-4- fluoro-2-(2- azaspiro[3.5]nonan-6- yl)-2H- benzo[d][1,2,3]triazole |
| 563 | 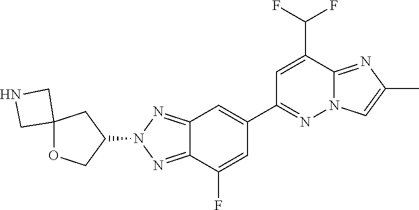 | (S)-7-(6-(8- (difluoromethyl)-2- methylimidazo[1,2- b]pyridazin-6-yl)-4- fluoro-2H- benzo[d][1,2,3]triazol-2- yl)-5-oxa-2- azaspiro[3.4]octane |
| 564 | 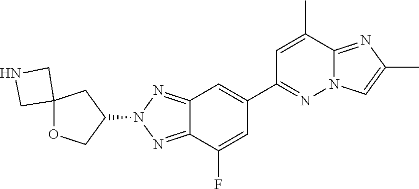 | (S)-7-(6-(2,8- dimethylimidazo[1,2- b]pyridazin-6-yl)-4- fluoro-2H- benzo[d][1,2,3]triazol-2- yl)-5-oxa-2- azaspiro[3.4]octane |
| 565 | 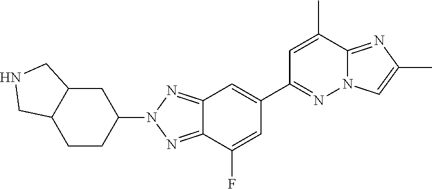 [SMILES: Cc1cn2nc(-c3cc(F)c4nn(C5CCC6CNCC6C5)nc4c3)cc(C)c2n1] | 6-(2,8- dimethylimidazo[1,2- b]pyridazin-6-yl)-4- fluoro-2-(octahydro-1H- isoindol-5-yl)-2H- benzo[d][1,2,3]triazole |
| 566 | 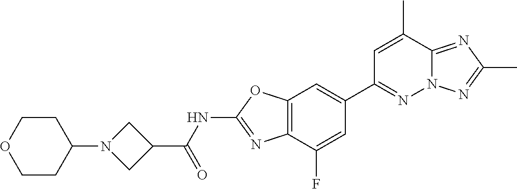 [SMILES: Cc1nc2c(C)cc(-c3cc(F)c4nc(NC(=O)C5CN(C6CCOCC6)C5)oc4c3)nn2n1] | N-(6-(2,8-dimethyl- [1,2,4]triazolo[1,5- b]pyridazin-6-yl)-4- fluorobenzo[d]oxazol-2- yl)-1-(tetrahydro-2H- pyran-4-yl)azetidine-3- carboxamide |
| 567 | 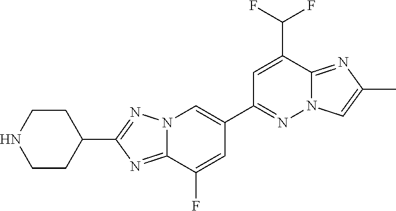 [SMILES: Cc1cn2nc(-c3cc(F)c4nc(C5CCNCC5)nn4c3)cc(C(F)F)c2n1] | 6-(8-(difluoromethyl)-2- methylimidazo[1,2- b]pyridazin-6-yl)-8- fluoro-2-(piperidin-4- yl)-[1,2,4]triazolo[1,5- a]pyridine |
| 568 | 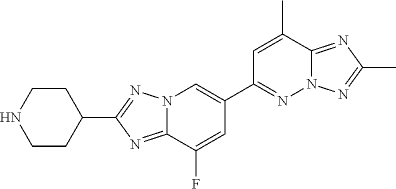 [SMILES: Cc1nc2c(C)cc(-c3cc(F)c4nc(C5CCNCC5)nn4c3)nn2n1] | 6-(8-fluoro-2-(piperidin- 4-yl)-[1,2,4]triazolo[1,5- a]pyridin-6-yl)-2,8- dimethyl- [1,2,4]triazolo[1,5- b]pyridazine |
| 569 | 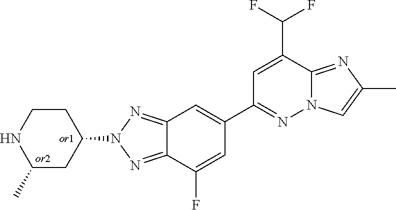 [SMILES: Cc1cn2nc(-c3cc(F)c4nn([C@H]5CCN[C@@H](C)C5)nc4c3)cc(C(F)F)c2n1] | rel-6-(8- (difluoromethyl)-2- methylimidazo[1,2- b]pyridazin-6-yl)-4- fluoro-2-((2R,4R)-2- methylpiperidin-4-yl)- 2H- benzo[d][1,2,3]triazole |
| 570 | 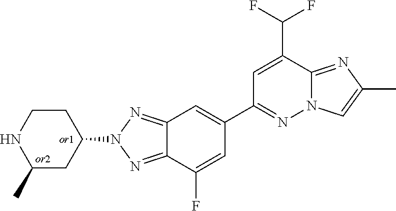 [SMILES: Cc1cn2nc(-c3cc(F)c4nn([C@H]5CCN[C@H](C)C5)nc4c3)cc(C(F)F)c2n1] | rel-6-(8- (difluoromethyl)-2- methylimidazo[1,2- b]pyridazin-6-yl)-4- fluoro-2-((2R,4S)-2- methylpiperidin-4-yl)- 2H- benzo[d][1,2,3]triazole |
| 571 | 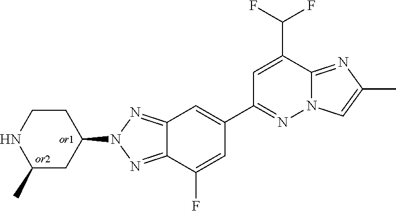 [SMILES: Cc1cn2nc(-c3cc(F)c4nn([C@@H]5CCN[C@H](C)C5)nc4c3)cc(C(F)F)c2n1] | rel-6-(8- (difluoromethyl)-2- methylimidazo[1,2- b]pyridazin-6-yl)-4- fluoro-2-((2R,4R)-2- methylpiperidin-4-yl)- 2H- benzo[d][1,2,3]triazole |
| 572 | 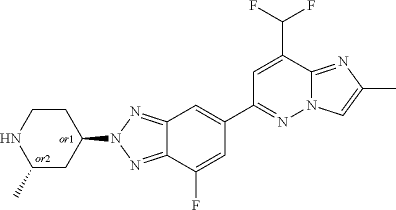 [SMILES: Cc1cn2nc(-c3cc(F)c4nn([C@@H]5CCN[C@@H](C)C5)nc4c3)cc(C(F)F)c2n1] | rel-6-(8- (difluoromethyl)-2- methylimidazo[1,2- b]pyridazin-6-yl)-4- fluoro-2-((2R,4S)-2- methylpiperidin-4-yl)- 2H- benzo[d][1,2,3]triazole |
| 573 | 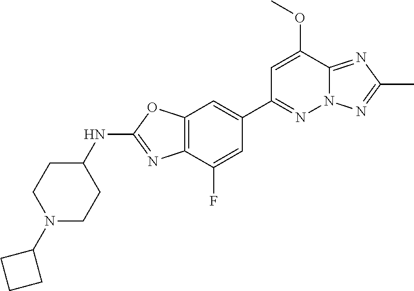 [SMILES: COc1cc(-c2cc(F)c3nc(NC4CCN(C5CCC5)CC4)oc3c2)nn2nc(C)nc12] | N-(1- cyclobutylpiperidin-4- yl)-4-fluoro-6-(8- methoxy-2-methyl- [1,2,4]triazolo[1,5- b]pyridazin-6- yl)benzo[d]oxazol-2- amine |
| 574 | 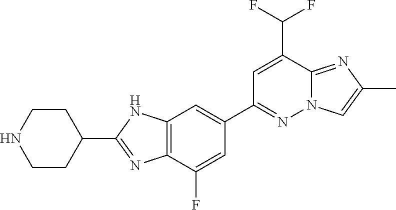 [SMILES: Cc1cn2nc(-c3cc(F)c4nc(C5CCNCC5)[nH]c4c3)cc(C(F)F)c2n1] | 8-(difluoromethyl)-6-(4- fluoro-2-(piperidin-4- yl)-1H- benzo[d]imidazol-6-yl)- 2-methylimidazo[1,2- b]pyridazine |
| 575 | 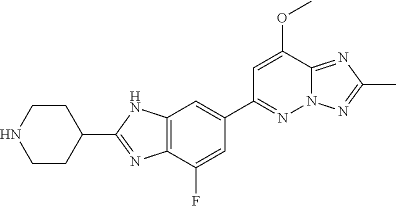 [SMILES: COc1cc(-c2cc(F)c3nc(C4CCNCC4)[nH]c3c2)nn2nc(C)nc12] | 6-(4-fluoro-2-(piperidin- 4-yl)-1H- benzo[d]imidazol-6-yl)- 8-methoxy-2-methyl- [1,2,4]triazolo[1,5- b]pyridazine |
| 576 | 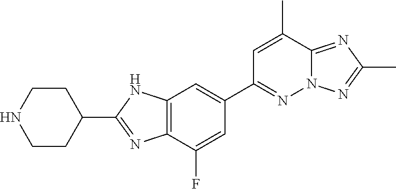 [SMILES: Cc1nc2c(C)cc(-c3cc(F)c4nc(C5CCNCC5)[nH]c4c3)nn2n1] | 6-(4-fluoro-2-(piperidin- 4-yl)-1H- benzo[d]imidazol-6-yl)- 2,8-dimethyl- [1,2,4]triazolo[1,5- b]pyridazine |
| 577 | 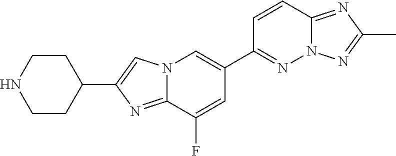 [SMILES: Cc1nc2ccc(-c3cc(F)c4nc(C5CCNCC5)cn4c3)nn2n1] | 6-(8-fluoro-2-(piperidin- 4-yl)imidazo[1,2- a]pyridin-6-yl)-2- methyl- [1,2,4]triazolo[1,5- b]pyridazine |

*stereochemistry arbitrarily assigned

### 2. Definitions

The term “halo” or “halogen,” as used herein, refers to fluoride, chloride, bromide, or iodide.

The term “alkyl” used alone or as part of a larger moiety, such as “alkoxy” or “haloalkyl” and the like, means saturated aliphatic straight-chain or branched monovalent hydrocarbon radical of formula —CₙH₍₂ₙ₊₁₎. Unless otherwise specified, an alkyl group typically has 1-20, 1-10 or 1-6 carbon atoms. In some embodiments, an alkyl group has 1-6 carbon atoms, i.e. C₁₋₆alkyl. As used herein, a “C₁₋₆alkyl” group means a radical having from 1 to 6 carbon atoms in a linear or branched arrangement. Examples include methyl, ethyl, n-propyl, iso-propyl, n-butyl, iso-butyl, tert-butyl, n-pentyl, isopentyl, hexyl, and the like. In some embodiments, an alkyl group has 1-4 carbon atoms, i.e., C₁₋₄alkyl. In some embodiments, an alkyl group has 1-3 carbon atoms, i.e., C₁₋₃alkyl.

The term “alkoxy” or “alkoxyl,” as used herein, refers to O-alkyl groups wherein alkyl is as defined above.

The term “haloalkyl” means alkyl, as the case may be, substituted with one or more halogen atoms. In one embodiment, the alkyl can be substituted by one to three halogens.

Examples of haloalkyl, include, but are not limited to, trifluoromethyl, trichloromethyl, pentafluoroethyl and the like.

The term “alkylene” as used herein, means a straight or branched chain divalent hydrocarbon group of formula —CₙH₂ₙ—. Non-limiting examples include ethylene, and propylene.

The term “carbocyclyl” refers to any stable non-aromatic hydrocarbon ring having 3-12 membered carbocyclyl. In one embodiment, carbocyclyl is 3-, 4-, 5-, 6-, 7-, or 8-membered monocyclic or bicyclic or 7-, 8-, 9-, 10-, 11-, or 12-membered bicyclic or tricyclic hydrocarbon ring, any of which may be saturated, partially unsaturated, or unsaturated. Any substitutable ring atom can be substituted (e.g., by one or more substituents). Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, and cyclooctadienyl. In one embodiment, carbocyclyl is intended to include, bridged, fused, and spirocyclic rings. In a spirocyclic carbocyclyl, one atom is common to two different rings. An example of a spirocyclic carbocyclyl is spiro[3.3]heptanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “bridged carbocyclyl” refers to a 5 to 12 membered polycyclic carbocyclyl group, wherein any two rings in the group share two disconnected atoms, the rings can have one or more double bonds but have no completely conjugated π-electron system. Representative examples of bridged carbocyclyl include, but are not limited to the following groups;

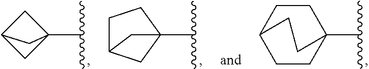

The term “cycloalkyl” refers to a monocyclic, bicyclic, tricyclic, or polycyclic saturated hydrocarbon groups having 3 to 12 ring carbons. In one embodiment, cycloalkyl may have 3 to 7 or 3 to 6 ring carbons. Any substitutable ring atom can be substituted (e.g., by one or more substituents). Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl include: bicyclo[1.1.0]butane, bicyclo[2.1.0]pentane, bicyclo[1.1.0]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heterocyclyl” or “heterocyclic” refers to a radical of a 3- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“3-12 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or tricyclic system (“tricyclic heterocyclyl”); polycyclic ring systems include fused, bridged, or spiro ring systems). Exemplary monocyclic heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system. Substituents may be present on one or more rings in the polycyclic ring system.

Spiro heterocyclyl refers to 5 to 12 membered polycyclic heterocyclyl with rings connected through one common carbon atom (called as spiro atom), wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C, wherein one or more rings may contain one or more double bonds, but none of the rings has a completely conjugated pi-electron system. Representative examples of spiro heterocyclyl include, but are not limited to the following groups:

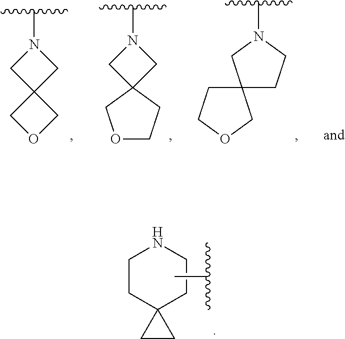

Fused heterocyclyl refers to a 5 to 12 membered polycyclic heterocyclyl group, wherein each ring in the group shares an adjacent pair of carbon atoms with another ring in the group, wherein one or more rings can contain one or more double bonds, but none of the rings has a completely conjugated π-electron system, and wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C. Representative examples of fused heterocyclyl include, but are not limited to the following groups:

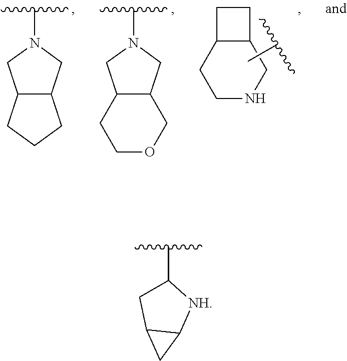

Bridged heterocyclyl refers to a 5 to 12 membered polycyclic heterocyclyl group, wherein any two rings in the group share two disconnected atoms, the rings can have one or more double bonds but have no completely conjugated π-electron system, and the rings have one or more heteroatoms selected from the group consisting of nitrogen, quaternary nitrogen, oxidized nitrogen (e.g., NO), oxygen, and sulfur, including sulfoxide and sulfone as ring atoms, the remaining ring atoms being C. Representative examples of bridged heterocyclyl include, but are not limited to the following groups:

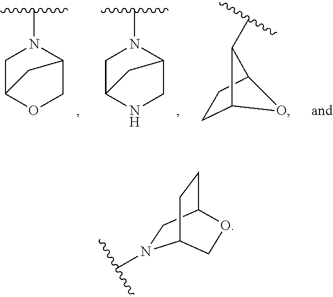

Generally, the carbocyclyl, the cycloalkyl, or the heterocyclyl may be unsubstituted, or be substituted with one or more substituents as valency allows, wherein the substituents can be independently selected from a number of groups. Exemplary substituents include but are not limited to, oxo, —CN, halogen, alkyl and alkoxyl, optionally, the alkyl substitution may be further substituted.

The term “aryl” refers to a 6 to 10 membered all-carbon monocyclic ring or a polycyclic fused ring (a “fused” ring system means that each ring in the system shares an adjacent pair of carbon atoms with other ring in the system) group, and has a completely conjugated π-electron system. The term “aryl” may be used interchangeably with the terms “aryl ring” “carbocyclic aromatic ring”, “aryl group” and “carbocyclic aromatic group”. Representative examples of aryl are phenyl and naphthyl.

The term “heteroaryl,” as used herein, refers to a monocyclic or multicyclic (e.g., bicyclic) aromatic hydrocarbon in which at least one of the ring carbon atoms has been replaced with a heteroatom independently selected from oxygen, nitrogen and sulfur. Preferably, the heteroaryl is based on a C₅₋₁₀ aryl with one or more of its ring carbon atoms replaced by the heteroatom. A heteroaryl group may be attached through a ring carbon atom or, where valency permits, through a ring nitrogen atom. Generally, the heteroaryl may be unsubstituted, or be substituted with one or more substituents as valency allows. Exemplary substituents include, but are not limited to, halogen, OH, alkyl, alkoxyl, and amino (e.g., NH₂, NHalkyl, N(alkyl)₂), optionally, the alkyl may be further substituted.

Examples of monocyclic 5-6 membered heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl), thienyl (e.g., 2-thienyl, 3-thienyl), pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl. A “substituted heteroaryl group” is substituted at any one or more substitutable ring atom, which is a ring carbon or ring nitrogen atom bonded to a hydrogen.

As used herein, many moieties (e.g., alkyl, alkylene, cycloalkyl, aryl, heteroaryl, or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Where suitable substituents are not specifically enumerated, exemplary substituents include, but are not limited to: C₁₋₅alkyl, C₁₋₅hydroxyalkyl, C₁₋₅haloalkyl, C₁₋₅alkoxy, C₁₋₅ haloalkoxy, halogen, hydroxyl, cyano, amino, —CN, —NO₂, —OR^{c1}, —NR^{a1}R^{b1}, —S(O)ᵢR^{a1}, —NR^{a1}S(O)ᵢR^{b1}, —S(O)NR^{a1}R^{b1}, —C(═O)OR^{a1}, —OC(═O)OR^{a1}, —C(═S)OR^{a1}, —O(C═S)R^{a1}, —C(═O)NR^{a1}R^{b1}, —NR^{a1}C(═O)R^{b1}, —C(═S)NR^{a1}R^{b1}, —C(═O)R^{a1}, —C(═S)R^{a1}, NR^{a1}C(═S)R^{b1}, —O(C═O)NR^{a1}R^{b1}, —NR^{a1}(C═S)OR^{b1}, —O(C═S)NR^{a1}R^{b1}, —NR^{a1}(C═O)NR^{a1}R^{b1}, —NR^{a1}(C═S)NR^{a1}R^{b1}, phenyl, or 5-6 membered heteroaryl. Each R^{a1} and each R^{b1} are independently selected from —H and C₁₋₅alkyl, optionally substituted with hydroxyl or C₁₋₃alkoxy; R^{c1} is —H, C₁₋₅haloalkyl or C₁₋₅alkyl, wherein the C₁₋₅alkyl is optionally substituted with hydroxyl or C₁-C₃alkoxy.

The symbol

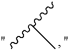

as used herein, refers to the point where the moiety attaches.

### Pharmaceutically Acceptable Salts

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Pharmaceutically acceptable salts of the compounds of any one of the formulae described above include acid addition and base salts.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Pharmaceutically acceptable salts of compounds of any one of the formulae described above may be prepared by one or more of three methods:
(i) by reacting the compound of any one of the formulae described above with the desired acid or base;
(ii) by removing an acid- or base-labile protecting group from a suitable precursor of the compound of any one of the formulae described above or by ring-opening a suitable cyclic precursor, for example, a lactone or lactam, using the desired acid or base; or
(iii) by converting one salt of the compound of any one of the formulae described above to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of any one of the formulae described above, and pharmaceutically acceptable salts thereof, may exist in unsolvated and solvated forms.

### Stereoisomers and Other Variations

The compounds of any one of the formulae described above may exhibit one or more kinds of isomerism (e.g. optical, geometric or tautomeric isomerism). Such variation is implicit to the compounds of any one of the formulae described above defined as they are by reference to their structural features and therefore within the scope of the present disclosure.

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When a compound is designated by its chemical name (e.g., where the configuration is indicated in the chemical name by “R” or “S”) or its structure (e.g., the configuration is indicated by “wedge” bonds) that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

When two stereoisomers are depicted by their chemical names or structures, and the chemical names or structures are connected by an “and”, a mixture of the two stereoisomers is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

Racemic mixture means 50% of one enantiomer and 50% of the corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

The term “geometric isomer” means isomers that differ in the orientation of substituent atoms in relationship to a carbon-carbon double bond, to a carbocyclic ring, or to a bridged bicyclic system. Substituent atoms (other than hydrogen) on each side of a carbon-carbon double bond may be in an E or Z configuration according to the Cahn-Ingold-Prelog priority rules. In the “E” configuration, the substituents having the highest priorities are on opposite sides in relationship to the carbon-carbon double bond. In the “Z” configuration, the substituents having the highest priorities are oriented on the same side in relationship to the carbon-carbon double bond.

Substituents around a carbon-carbon double bond can also be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

Where structural isomers are interconvertible via a low energy barrier, tautomeric isomerism (“tautomerism”) can occur. This can take the form of proton tautomerism in compounds of any one of the formulae described above containing, for example, an imino, keto, or oxime group, or so-called valence tautomerism in compounds which contain an aromatic moiety. It follows that a single compound may exhibit more than one type of isomerism.

In certain instances tautomeric forms of the disclosed compounds exist, such as the tautomeric structures shown below:

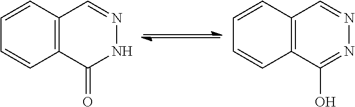 [SMILES: O=c1[nH]ncc2ccccc12 ; Oc1nncc2ccccc12]

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

Cis/trans isomers may be separated by conventional techniques well known to those skilled in the art, for example, chromatography and fractional crystallisation.

Conventional techniques for the preparation/solation of individual enantiomers/diastereomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of any one of the formulae described above contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of any one of the formulae described above (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). Columns can be obtained from Chiral Technologies, Inc, West Chester, Pa., USA, a subsidiary of Daicel® Chemical Industries, Ltd., Tokyo, Japan.

It must be emphasized that the compounds of any one of the formulae described above have been drawn herein in a single tautomeric form, all possible tautomeric forms are included within the scope of the present disclosure.

### 3. Administration and Dosing

Typically, a compound of the present disclosure is administered in an amount effective to treat a condition as described herein. The compounds of the present disclosure can be administered as compound per se, or alternatively, as a pharmaceutically acceptable salt. For administration and dosing purposes, the compound per se or pharmaceutically acceptable salt thereof will simply be referred to as the compounds of the present disclosure.

The compounds of the present disclosure are administered by any suitable route in the form of a pharmaceutical composition adapted to such a route, and in a dose effective for the treatment intended. The compounds of the present disclosure may be administered orally, rectally, vaginally, parenterally, or topically.

The compounds of the present disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the present disclosure may also be administered directly into the bloodstream, into muscle, or into an internal organ. Suitable means for parenteral administration include intravenous, intra-arterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the present disclosure may also be administered topically to the skin or mucosa, that is, dermally or transdermally. In another embodiment, the compounds of the present disclosure can also be administered intranasally or by inhalation. In another embodiment, the compounds of the present disclosure may be administered rectally or vaginally. In another embodiment, the compounds of the present disclosure may also be administered directly to the eye or ear.

The dosage regimen for the compounds of the present disclosure and/or compositions containing said compounds is based on a variety of factors, including the type, age, weight, sex and medical condition of the patient; the severity of the condition; the route of administration; and the activity of the particular compound employed. Thus the dosage regimen may vary widely. In one embodiment, the total daily dose of a compound of the present disclosure is typically from about 0.001 to about 100 mg/kg (i.e., mg compound of the present disclosure per kg body weight) for the treatment of the indicated conditions discussed herein.

For oral administration, the compositions may be provided in the form of tablets containing 0.1-500 milligrams of the active ingredient for the symptomatic adjustment of the dosage to the patient. A medicament typically contains from about 0.01 mg to about 500 mg of the active ingredient. Intravenously, doses may range from about 0.01 to about 10 mg/kg/minute during a constant rate infusion.

Suitable subjects according to the present disclosure include mammalian subjects, including non-human mammal such as primates, rodents (mice, rats, hamsters, rabbits etc).

In one embodiment, humans are suitable subjects. Human subjects may be of either gender and at any stage of development.

### 4. Pharmaceutical Compositions

In another embodiment, the present disclosure comprises pharmaceutical compositions. Such pharmaceutical compositions comprise a compound of the present disclosure presented, a pharmaceutically acceptable salt, or a stereoisomer thereof with a pharmaceutically acceptable carrier or excipient. Other pharmacologically active substances can also be present.

As used herein, “pharmaceutically acceptable carrier or excipient” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Pharmaceutically acceptable substances such as wetting agents or minor amounts of auxiliary substances such as wetting or emulsifying agents, preservatives or buffers, which enhance the shelf life or effectiveness of the antibody or antibody portion.

The compositions of present disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the antibody is administered by intravenous infusion or injection. In yet another embodiment, the antibody is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the present disclosure. In another embodiment, the oral administration may be in a powder or granule form. In another embodiment, the oral dose form is sub-lingual, such as, for example, a lozenge. In such solid dosage forms, the compounds of any one of the formulae described above are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present disclosure comprises a parenteral dose form.

“Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present disclosure comprises a topical dose form.

“Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of present disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88:955-958, 1999.

Formulations suitable for topical administration to the eye include, for example, eye drops wherein the compound of present disclosure is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the present disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present disclosure comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the present disclosure may be prepared by any of the well-known techniques of pharmacy, such as effective formulation and administration procedures.

The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### 5. Method of Treatment

The terms “subject,” “individual,” or “patient,” used interchangeably, refer to any animal, including mammals, preferably mice, rats, other rodents, rabbits, dogs, cats, swine, cattle, sheep, horses, or primates, and most preferably humans.

The terms “treatment,” “treat,” and “treating” refer to reversing, alleviating, or inhibiting the progress of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment) (e.g., in light of a history of symptoms and/or in light of exposure to a pathogen). Treatment may also be continued after symptoms have resolved, for example, to delay or prevent recurrence.

The term “prevention” (or “prevent” or “preventing”), as used herein, refers to precluding, averting, obviating, forestalling, reducing the incidence of, stopping, or hindering the symptoms of a disease, disorder and/or condition. Prevention includes administration to a subject who does not exhibit symptoms of a disease, disorder, and/or condition at the time of administration.

The terms “condition,” “disease,” and “disorder” are used interchangeably.

The term “administer,” “administering,” or “administration” refers to methods introducing a compound disclosed herein, or a composition thereof, in or on a patient. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

Generally, an effective amount of a compound taught herein varies depending upon various factors, such as the given drug or compound, the pharmaceutical formulation, the route of administration, the type of disease or disorder, the identity of the subject or host being treated, and the like, but can nevertheless be routinely determined by one skilled in the art. An effective amount of a compound of the present teachings may be readily determined by one of ordinary skill by routine methods known in the art.

The term “therapeutically effective amount” means an amount when administered to the subject which results in beneficial or desired results, including clinical results, e.g., inhibits, suppresses or reduces the symptoms of the condition being treated in the subject as compared to a control. The exact amount required will vary from subject to subject, depending on the species, age, and general condition of the subject, the severity of the disease, the particular anticancer agent, its mode of administration, combination treatment with other therapies, and the like.

The present disclosure is directed to compounds of formula (I) (including all its embodiments), which are useful in the treatment and/or prevention of a disease and/or condition associated with or modulated by HTT, especially wherein lowering mHTT in a subject is of therapeutic benefit, including but not limited to the treatment and/or prevention of HD.

In one embodiment, the present disclosure relates to a compound of formula (I) or a pharmaceutically acceptable salt thereof for use as a medicament.

In one embodiment, the present disclosure relates to a compound of (I) or a pharmaceutically acceptable salt thereof for use in a method of treatment of the human or animal body.

The present disclosure further provides a method of treating HD in a subject in need thereof, comprising administering to the subject an effective amount of (1) a compound of Formula (I) or a pharmaceutically acceptable salt thereof; or (2) a pharmaceutically acceptable composition comprising a compound of Formula (I) or a pharmaceutically acceptable salt thereof, and a pharmaceutically acceptable carrier.

In one embodiment, the present disclosure provides a use for a compound of Formula (I) or a pharmaceutically acceptable salt thereof for treating HD in a subject in need thereof comprising, administering to the subject an effective amount of the compound of Formula (I) or a pharmaceutically acceptable salt thereof.

In one embodiment, the present disclosure provides a use for a compound of Formula (I) or a pharmaceutically acceptable salt thereof in the manufacture of a medicament for treating HD in a subject in need thereof comprising, administering to the subject an effective amount of the medicament.

### 6. Treatment Kits

One aspect of the present invention relates to a kit for conveniently and effectively carrying out the methods or uses in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope. Indeed, various modifications of the invention, and many further embodiments thereof, in addition to those shown and described herein, will become apparent to those skilled in the art upon review of this document, including the examples which follow and the references to the scientific and patent literature cited herein.

The contents of the cited references are incorporated herein by reference to help illustrate the state of the art.

In addition, for purposes of this invention, the chemical elements are identified in accordance with the Periodic Table of the Elements, CAS version, Handbook of Chemistry and Physics, 75^{th}Ed., inside cover. Additionally, general principles of organic chemistry, as well as specific functional moieties and reactivity, are described in “Organic Chemistry,” Thomas Sorrell, University Science Books, Sausalito: 1999, and “Organic Chemistry,” Morrison & Boyd (3d Ed), the entire contents of both of which are incorporated herein by reference.

### 7. Preparation

The compounds of any one of the formulae described above, may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds of any one of the formulae described above, it is noted that some of the preparation methods described herein may require protection of remote functionality (e.g., primary amine, secondary amine, carboxyl in any one of the formulae described above precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines, and lower alkyl or benzyl esters for carboxylic acids) which are generally not chemically reactive under the reaction conditions described and can typically be removed without chemically altering other functionality in the any one of the formulae described above compounds.

The Schemes described below are intended to provide a general description of the methodology employed in the preparation of the compounds of the present disclosure. Some of the compounds of the present disclosure may contain single or multiple chiral centers with the stereochemical designation (R) or (S). It will be apparent to one skilled in the art that all of the synthetic transformations can be conducted in a similar manner whether the materials are enantio-enriched or racemic. Moreover, the resolution to the desired optically active material may take place at any desired point in the sequence using well known methods such as described herein and in the chemistry literature.

### EXAMPLES

| Abbreviation | Meaning |
|---|---|
| ACN or CH₃CN or MeCN | acetonitrile |
| AcOH | Acetic acid |
| BuOH | n-butanol |
| ° C | Degrees centigrade |
| celite | Diatomaceous earth |
| Cl | Chloride |
| Cs₂CO₃ | Cesium carbonate |
| d/h/hr/hrs/min/s | Day/hour/minute/second |
| d | doublet |
| D | Deuterium |
| dd | Doublet of doublets |
| DCE | Dichloroethane |
| DCM or CH₂Cl₂ | dichloromethane |
| DIPEA | N,N-diisopropylamine |
| DMF | dimethylformamide |
| DMSO | dimethylsulfoxide |
| EtOAc | Ethyl acetate |
| EtOH | ethanol |
| Eq. | equivalent |
| FA | Formic acid |
| H₂ | Hydrogen |
| HBr | Hydrobromic acid |
| HCl | Hydrochloric acid |
| HPLC | High performance liquid chromatography |
| J | Coupling constant |
| K₂CO₃ | Potassium carbonate |
| KOH | Potassium hydroxide |
| LC/MS or LCMS | Liquid chromatographic mass spectroscopy |
| m | multiplet |
| MTBE | Methyl tertiary-butyl ether |
| MeOH | methanol |
| MgSO₄ | Magnesium sulfate |
| m/z | Mass to charge ratio |
| NaH | Sodium hydride |
| NEt₃ or Et₃N or TEA | Triethylamine |
| Na₂SO₄ | Sodium sulfate |
| N₂ | nitrogen |
| NMR | Nuclear magnetic resonance |
| Pd | palladium |
| Pd/C | Palladium on carbon |
| PE | Petroleum ether |
| ppm | Parts per million |
| rt | Room temperature |
| RT | Retention time |
| s | singlet |
| t | triplet |
| TFA | Trifluoroacetic acid |
| TFAA | Trifluoroacetic anhydride |
| THF | tetrahydrofuran |
| TLC | Thin layer chromatography |
| t-Bu | Tert-butyl |
| Ts | toluenesulfonyl |

### Section 1. General Methods and Analytical Methods

### a. General Methods

The compounds of the Examples were analyzed or purified according to one of the Purification Methods referred to below unless otherwise described. Where preparative TLC/HPLC or silica gel chromatography have been used, one skilled in the art may choose any combination of solvents to purify the desired compound. Silica gel column chromatography was performed using 20-40 mM (particle size), 250-400 mesh, or 400-632 mesh silica gel using either a Teledyne ISCO Combiflash RF or a Grace Reveleris X2 with ELSD purification systems or using pressurized nitrogen (˜10-15 psi) to drive solvent through the column (“flash chromatography”). Wherein an SCX column has been used, the eluant conditions are MeOH followed by methanolic ammonia. Where indicated, solutions and reaction mixtures were concentrated by rotary evaporation under vacuum.

### b. Analytical Methods

### Analytical LCMS Instrumentation Specifications:

Waters Acquity iClass UPLC with QDa mass spectrometer and PDA (photodiode array detector)

### RxnQC/FrxQC/PurityQC Analysis LC/MS Method Conditions:

### Ammonium Hydroxide (Basic pH) Conditions

### Method 1

MS mode: MS:ESI+ scan range 165-650 daltons
PDA: 200-400 nm scan range
Column: Waters ACQUITY UPLC BEH C18 2.1×50 mm, 1.7 um; Part No. 186002350
Modifier: Ammonium hydroxide 0.2% (v/v) conc.
Method: 95% H₂O/5% MeCN (initial conditions) hold 0.1 min, linear gradient to 5% H₂O/95% MeCN at
3.25 min, hold 5% H₂O/95% MeCN to 3.5 min. Flow rate, 0.8 mL/min.

### Method 2

MS mode: MS:ESI+ scan range 165-650 daltons
PDA: 200-400 nm scan range
Column: Waters ACQUITY UPLC BEH C18 2.1×30 mm, 1.7 um; Part No. 186002349
Modifier: Ammonium hydroxide 0.2% (v/v) conc.
Method: 95% H₂O/5% MeCN (initial conditions), linear gradient to 5% H₂O/95% MeCN at 1.0 min, hold 5% H₂O/95% MeCN to 1.3 min. Flow rate, 0.7 mL/min.

### Trifluoroacetic Acid (Acidic pH) Conditions

### Method 3

MS mode: MS:ESI+ scan range 165-650 daltons
PDA: 200-400 nm scan range
Column: Waters ACQUITY UPLC BEH C18 2.1×50 mm, 1.7 um; Part No. 186002350
Modifier: Trifluoroacetic acid 0.1% (v/v) conc.
Method: 95% H₂O/5% MeCN (initial conditions) hold 0.1 min, linear gradient to 5% H₂O/95% MeCN at
3.25 min, hold 5% H₂O/95% MeCN to 3.5 min. Flow rate, 0.8 mL/min.

### Method 4

MS mode: MS:ESI+ scan range 165-650 daltons
PDA: 200-400 nm scan range
Column: Waters ACQUITY UPLC BEH C18 2.1×50 mm, 1.7 um; Part No. 186002349
Modifier: Trifluoroacetic acid 0.1% (v/v) conc.
Method: 95% H₂O/5% MeCN (initial conditions), linear gradient to 5% H₂O/95% MeCN at 1.0 min, hold 5% H₂O/95% MeCN to 1.3 min. Flow rate, 0.7 mL/min.

### Analytical LCMS Instrumentation Specifications:

Agilent 1200 Series LC/MSD system with DAD\ELSD Alltech 3300 and Agilent LC\MSD G6130A, G6120B mass-spectrometer; Agilent Technologies 1260 Infinity LC/MSD system with DAD\ELSD Alltech 3300 and Agilent LC\MSD G6120B mass-spectrometer; Agilent Technologies 1260 Infinity II LC/MSD system with DAD\ELSD G7102A 1290 Infinity II and Agilent LC\MSD G6120B mass-spectrometer; Agilent 1260 Series LC/MSD system with DAD\ELSD and Agilent LC\MSD (G6120B) mass-spectrometer; UHPLC Agilent 1290 Series LC/MSD system with DAD\ELSD and Agilent LC\MSD (G6125B) mass-spectrometer.

### RxnQC/FrxQC/PurityQC Analysis LC/MS Method Conditions:

### Formic Acid (Acidic pH) Conditions

### Method 5

Inject volume: 0.5 μl, Column Temperature: 60° C., UV scan: 207-223 nM, 246-262 nM, 272-288 nM, Agilent Poroshell 120 SB-C18 4.6×30 mm 2.7 μm with UHPLC Guard Infinity Lab Poroshell 120 SB-C18 4.6×5 mm 2.7 μm, Mobile phase A: 0.1% FA in Water, Mobile phase
B: 0.1% FA in Acetonitrile

### Details of Elution

| Time (min) | Flow (mL/min) | % A | % B |
|---|---|---|---|
| 0.00 | 3.00 | 99 | 1 |
| 0.01 | 3.00 | 99 | 1 |
| 1.5 | 3.00 | 0 | 100 |
| 1.73 | 3.00 | 0 | 100 |
| 1.74 | 3.00 | 99 | 1 |

### Method 6

Inject volume: 0.5 μl; Column Temperature: 60° C.; UV scan: 207-223 nM, 246-262 nM, 272-288 nM
Agilent Poroshell 120 SB-C18 4.6×30 mm 2.7 pm with UHPLC Guard Infinity Lab Poroshell 120 SB-C18 4.6×5 mm 2.7 pm, Mobile phase A: 0.1% FA in Water, Mobile phase B: 0.1% FA in Acetonitrile

### Details of Elution

| Time (min) | Flow (mL/min) | % A | % B |
|---|---|---|---|
| 0.00 | 1.5 | 99 | 1 |
| 0.01 | 1.5 | 99 | 1 |
| 5.00 | 1.5 | 0 | 100 |
| 5.99 | 1.5 | 0 | 100 |
| 6.00 | 1.5 | 99 | 1 |

### Method 7

MS mode: MS ESI+ scan range 100-1000 daltons
PDA: 190-370 nm scan range
Column: Xtimate C18 2.1V*30 mm, 3 um
Modifier: A Phase: water (4 L)+TFA (1.5 mL), B Phase: acetonitrile (4 L)+TFA (0.75 mL)
Method: using the elution gradient 10%-80% (solvent B) over 1.35 minutes and holding at 80% for 0.9 minutes at a flow rate of 0.8 ml/min.

### Method 8

5-95AB_4 min · M

| Instrument | | Agilent 1260\G6125B | | |
|---|---|---|---|---|
| Software | | Agilent ChemStation Rev. C. 01.10[201] | | |
| HPLC | Column | Shim-pack Velox SP-C18 2.7 μm 3.0*30 | | |
| | | mm | | |
| | Mobile Phase | A: 0.0375% TFA in water (v/v) | | |
| | | B: 0.01875% TFA in ACN (v/v) | | |
| | Gradient | Time(min) | B(%) | Flow(mL/min) |
| | | 0.0 | 5 | 1.0 |
| | | 3.00 | 95 | 1.0 |
| | | 3.60 | 95 | 1.5 |
| | | 3.61 | 5 | 1.5 |
| | | 4.00 | 5 | 1.5 |

| | | |
|---|---|---|
| | Column Temp | 50° C. |
| | Detector | DAD&ELSD |
| MS | Ionization source | ESI |
| | Drying Gas | N2 |
| | Drying Gas Flow | 10(L/min) |
| | Nebulizer | 40(psig) |
| | Pressure | |
| | Drying Gas Temp | 350(° C.) |
| | Capillary Voltage | 4000(V) |
| | MS Polarity | Positive |
| | MS Mode | Scan |

### Description

Mobile phase: Ramp from 5% ACN (0.018% TFA) in water (0.037% TFA) to 95% ACN in 3.0 min, Flow rate is set at 1.0 mL/min; then hold at 95% ACN for 0.60 minutes Flow rate is set from 1.0 mL/min to 1.5 mL/min; return back to 5% ACN in water and hold for 0.40 m.

Flow rate is set at 1.5 mL/min.

Column temperature at 50′° C.

The column is of Shim-pack Velox SP-C18 2.7 μm 3.0*30 mm.

### Method 9

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.0 | 5 | 2.0 |
| | | 0.60 | 95 | 2.0 |
| | | 0.78 | 95 | 2.0 |
| | | 0.79 | 5 | 2.0 |
| | | 0.80 | 5 | 2.0 |

| | | |
|---|---|---|
| | Column Temp | 50° C. |
| | Detector | PDA |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |

### Description

Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

Column temperature at 50° C.

The column is of Kinetex® EVO C18 2.1×30 mm 5 um.

### Method 10

### Description

Mobile phase: Ramp from 5% ACN (0.01875% TFA) in water (0.0375% TFA) to 95% ACN in 3.20 min, Flow rate is set at 1.5 mL/min; then hold at 95% ACN for 0.30 minutes Flow rate is set at 1.5 mL/min; return back to 5% ACN in water and hold for 0.30 min. Flow rate is set at 2.0 mL/min. Column temperature at 50° C. The column is of Kinetex® EVO C18 4.6×50 mm 5 um.

### Preparative HPLC-MS Conditions:

### HPLC-MS Instrumentation Specifications

Waters Autopurification with QDa mass spectrometer and PDA (photodiode array detector)

### Ammonium Hydroxide (Basic pH) Conditions

Flow rate: 30 mL/min
MS mode: MS:ESI+ scan range 165-650 daltons
PDA: 200-400 nm scan range
Column: Waters XSELECT CSH C18 PREP 19×100 mm, 5 um; Part No. 186005421
Modifier: 0.2% Ammonium hydroxide (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.
Flow rate: 50 mL/min
MS mode: MS:ESI+ scan range 165-650 daltons
PDA: 200-400 nm scan range
Column: Waters XSELECT CSH C18 PREP 30×100 mm, 5 um; Part No. 186005425
Modifier: 0.2% Ammonium hydroxide (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.
Flow rate, 60 mL/min
MS mode: MS:ESI+ scan range 165-650 daltons
PDA: 200-400 nm scan range
Column: Waters XSELECT CSH C18 PREP 30×50 mm, 5 um; Part No. 186005423
Modifier: 0.2% Ammonium hydroxide (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.
Column: Boston Prime C18 150×30 mm×5 um; Condition: water (NH₃H₂O+NH₄HCO₃)-ACN; Gradient (% organic): 0-100% optimized for each example; Flow Rate (mL/min) 25.
Column: YMC Actus Trial C18 20*100 5 mkm column; gradient mixture H₂O-MeOH-Ammonia 0.1% as a mobile phase optimized for each example.

### Trifluoroacetic Acid (Acidic pH) Conditions

Flow rate, 30 mL/min
MS mode: MS:ESI+ scan range 165-650 daltons
PDA: 200-400 nm scan range
Column: Waters Sunfire OBD C18 PREP 19×100 mm, 5 um; Part No. 186002567
Modifier: 0.1% Trifluoroacetic acid (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.
Flow rate, 50 mL/min
MS mode: MS:ESI+ scan range 165-650 daltons
PDA: 200-400 nm scan range
Column: Waters Sunfire OBD C18 PREP 30×100 mm, 5 um; Part No. 186002572
Modifier: 0.1% Trifluoroacetic acid (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.
Flow rate, 60 mL/min
MS mode: MS:ESI+ scan range 165-650 daltons
PDA: 200-400 nm scan range
Column: Waters Sunfire OBD C18 PREP 30×50 mm, 5 um; Part No. 186002570
Modifier: 0.1% Trifluoroacetic acid (v/v) conc.
Method: A % H₂O/B % MeCN (initial conditions) hold 0.5 min, linear gradient to A % H₂O/B % MeCN at 8 min, ramp to 5% H₂O/95% MeCN at 8.5 min, HOLD 5% H₂O/95% MeCN to 10 min.

### Formic Acid (FA, Acidic pH) Conditions

Column: Welch Xtimate C18 150×30 mm×5 um; Condition: water (FA)-ACN; Gradient (% organic): optimized for each example; Flow Rate (mL/min) 25.

### Hydrochloric Acid (HCl, Acidic pH) Conditions

Column: Boston Green ODS 150×30 mm×5 um; Condition: water (HCl)-ACN; Gradient (% organic): 0-100% optimized for each example; Flow Rate (m/min) 25.

### Analytical SFC Instrumentation Specifications

Waters Acquity UPC² SFC with QDa mass spectrometer and PDA (photodiode array detector)

### Analytical Screening Conditions

MS mode: MS:ESI+ scan range 100-650 daltons
PDA: 200-400 nm scan range
Columns: See below
Solvent: Airgas Bone Dry Co₂
Cosolvents: Methanol, Ethanol, or Isopropanol with either 0.1% Diethylamine, 0.1% Dimethylethanolamine, or neutral
Method: Isocratic conditions; typically 60% CO₂: 40% cosolvent or 70% CO₂: 30% cosolvent, Flow rate, 3.0 mL/min.

### Preparative SFC Instrumentation Specifications

Waters Prep100 SFC with QDa mass spectrometer, PDA (photodiode array detector,) and 2767 Collection bed

### Preparative Conditions

Method: X % Cosolvent w/Y % modifier in CO₂, isocratic conditions.
Flow rate: 100 mL/min
Automated back pressure regulator: 120 bar
Manual back pressure regulator: 40 psi for MeOH or EtOH, 60 psi for iPrOH
Column oven temperate: 40° C.
MS mode: MS:ESI+ scan range 150-650 daltons
PDA: 200-400 nm scan range

### SFC Columns, Analytical:

AD-H: Daicel Chiralpak AD-H, 4.6 mm×250 mm, 5 um, Part No 19325
AS-H: Daicel Chiralpak AS-H, 4.6 mm×250 mm, 5 um, Part No 20325
OD-H: Daicel Chiralpak OD-H, 4.6 mm×250 mm, 5 um, Part No 14325
OX-H: Daicel Chiralpak OX-H, 4.6 mm×250 mm, 5 um, Part No 63325
IA: Daicel Chiralpak IA, 4.6 mm×250 mm, 5 um, Part No 80325
IB: Daicel Chiralpak IB, 4.6 mm×250 mm, 5 um, Part No 81325
IC: Daicel Chiralpak IC, 4.6 mm×250 mm, 5 um, Part No 83325
IG: Daicel Chiralpak IG, 4.6 mm×250 mm, 5 um, Part No 87325
Cell-2: Phenomenex Lux Cellulose-2, 4.6 mm×150 mm, 3 um, Part No. 00F-4456-E0
Cell-4: Phenomenex Lux Cellulose-4, 4.6 mm×150 mm, 3 um, Part No. 00F-4490-E0

### SFC Columns, Preparative:

AD-H: Daicel Chiralpak AD-H, 30 mm×250 mm, 5 um, Part No 19475
AS-H: Daicel Chiralpak AS-H, 30 mm×250 mm, 5 um, Part No 20475
OD-H: Daicel Chiralpak OD-H, 30 mm×250 mm, 5 um, Part No 14475
OX-H: Daicel Chiralpak OX-H, 30 mm×250 mm, 5 um, Part No 63475
IA: Daicel Chiralpak IA, 30 mm×250 mm, 5 um, Part No 80475
IB: Daicel Chiralpak IB, 30 mm×250 mm, 5 um, Part No 81475
IC: Daicel Chiralpak IC, 30 mm×250 mm, 5 um, Part No 83475
IG: Daicel Chiralpak IG, 30 mm×250 mm, 5 um, Part No 87475
Cell-2: Phenomenex Lux Cellulose-2, 30 mm×250 mm, 5 um, Part No. 00G-4457-U0-AX
Cell-4: Phenomenex Lux Cellulose-4, 30 mm×250 mm, 5 um, Part No. 00G-4491-U0-AX

### ¹H-NMR

¹H nuclear magnetic resonance (NMR) spectra were in all cases consistent with the proposed structures. The ¹H NMR spectra were recorded on a Bruker Avance III HD 500 MHz, Bruker Avance III 500 MHz, Bruker Avance DRX 500, Bruker Avance III 400 MHz, Varian-400 VNMRS, Varian Unityplus 400, or Varian-400 MR. Characteristic chemical shifts (d) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteromethanol. Where appropriate, tautomers may be recorded within the NMR data; and some exchangeable protons may not be visible.

### Section 2. Preparation of Intermediates

### Intermediate 1

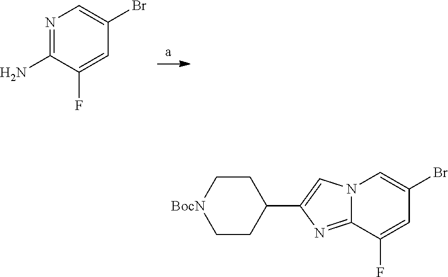 [SMILES: Nc1ncc(Br)cc1F ; CC(C)(C)OC(=O)N1CCC(c2cn3cc(Br)cc(F)c3n2)CC1]

A solution of 5-bromo-3-fluoro-pyridin-2-amine (623.79 mg, 3.27 mmol) and tert-butyl 4-(2-bromo-acetyl)piperidine-1-carboxylate (1 g, 3.27 mmol) in water (30 mL) was stirred at 60° C. for 12 hours. The mixture was concentrated to give the residue, which was purified by flash silica gel chromatography (EtOAc in petroleum ether from 0-25%) to give tert-butyl 4-(6-bromo-8-fluoro-imidazo[1,2-a]pyridin-2-yl)piperidine-1-carboxylate (900 mg, 2.26 mmol, 69% yield) as a light-yellow solid. MS: m/z 297.9 [M+H]⁺; ¹H NMR (400 MHz, CHLOROFORM-d) δ ppm: 8.03 (s, 1H), 7.34 (d, J=2.7 Hz, 1H), 6.97 (dd, J=9.5, 1.2 Hz, 1H), 4.12-4.27 (m, 2H), 2.79-2.97 (m, 3H), 2.06 (br d, J=13.0 Hz, 2H), 1.64 (br dd, J=12.3, 2.8 Hz, 2H), 1.43-1.46 (m, 9H).

### Intermediate 2

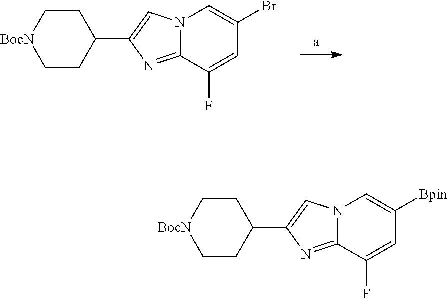

To a solution of tert-butyl 4-(6-bromo-8-fluoroimidazo[1,2-a]pyridin-2-yl)piperidine-1-carboxylate (4 g, 10.04 mmol) and 4,4,5,5-tetramethyl-2-(4,4,5,5-tetramethyl-1,3,2-dioxaborolan-2-yl)-1,3,2-dioxaborolane (5.10 g, 20.09 mmol) in Dioxane (150 mL) was added cyclopentyl(diphenyl)phosphane dichloromethane dichloropalladium; iron (820.19 mg, 1.00 mmol) and KOAc (2.96 g, 30.13 mmol) and was stirred at 100° C. for 48 hours under N₂ atmosphere. The mixture was filtered and concentrated to give the residue. The residue was purified by flash silica gel chromatography (Petroleum ether/EtOAc=3/1) to give tert-butyl 4-[8-fluoro-6-(4,4,5,5-tetramethyl-1,3,2-dioxaborolan-2-yl)imidazo[1,2-a]pyridin-2-yl]piperidine-1-carboxylate (3.12 g, 7.01 mmol, 69% yield) as a yellow solid. MS: m/z 446.3 [M+H]⁺

### Intermediate 3

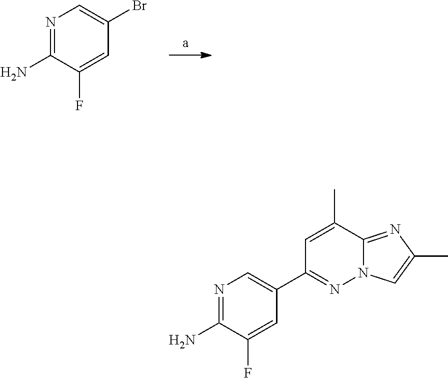 [SMILES: Nc1ncc(Br)cc1F ; Cc1cn2nc(-c3cnc(N)c(F)c3)cc(C)c2n1]

To a solution of 5-bromo-3-fluoro-pyridin-2-amine (480 mg, 2.51 mmol) and (2,8-dimethylimidazo[1,2-b]pyridazin-6-yl)boronic acid (480 mg, 2.51 mmol) in dioxane (5 mL) and water (1 mL) was added K₂CO₃ (694 mg, 5.03 mmol) and Pd(dppf)Cl₂ (367 mg, 0.50 mmol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified by flash silica gel chromatography (40% to 100% EtOAc:PE) to give 5-(2,8-dimethylimidazo[1,2-b]pyridazin-6-yl)-3-fluoro-pyridin-2-amine (446 mg, 1.73 mmol, 68% yield) as a yellow solid. MS: m/z 258.1 [M+H]⁺, ¹H NMR (400 MHz, DMSO-d₆) δ 8.48 (s, 1H), 7.97-7.92 (m, 2H), 7.59 (s, 1H), 6.72 (s, 2H), 2.56 (s, 3H), 2.38 (s, 3H).

### Intermediate 4

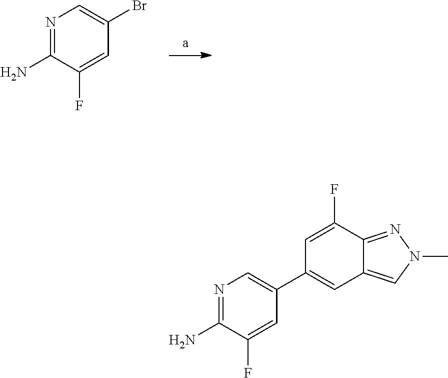 [SMILES: Nc1ncc(Br)cc1F ; Cn1cc2cc(-c3cnc(N)c(F)c3)cc(F)c2n1]

To a solution of 7-fluoro-2-methyl-5-(4,4,5,5-tetramethyl-1,3,2-dioxaborolan-2-yl)-2H-indazole (3.18 g, 11.52 mmol) in Dioxane (60 mL), water (20 mL) was added 5-bromo-3-fluoropyridin-2-amine (2 g, 10.47 mmol), tripotassium phosphate (4.45 g, 20.94 mmol) and cyclopentyl(diphenyl)phosphane dichloropalladium; iron (766.18 mg, 1.05 mmol). The mixture was stirred at 100° C. for 12 h under N₂ atmosphere. The mixture was concentrated and then water (50 mL) was added. The mixture was extracted with EtOAc (50 mL×3). The combined organic layers were washed with brine (100 mL), dried over Na₂SO₄, filtered and concentrated. The crude material was purified by flash silica gel chromatography (from CH₂Cl₂: MeOH=100/1 to 20/1) to yield 3-fluoro-5-(7-fluoro-2-methyl-2H-indazol-5-yl)pyridin-2-amine (1.77 g, 6.78 mmol, 64.72% yield, 99% purity) as yellow solid. MS: m/z 261.0 [M+H]⁺, ¹H NMR (400 Hz, DMSO-d6) δ 8.46 (d, J=2.8 Hz, 1H), 8.17 (d, J=1.6 Hz, 1H), 7.73-7.80 (m, 2H), 7.35-7.39 (m, 1H), 6.33 (s, 2H), 4.20 (s, 3H).

### Intermediate 5

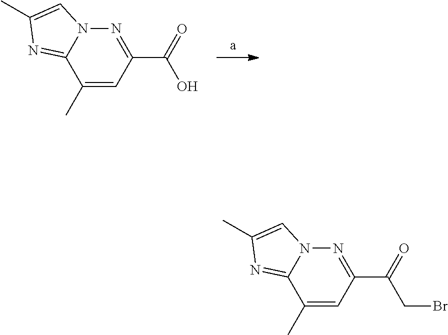

To a solution of 2,8-dimethylimidazo[1,2-b]pyridazine-6-carboxylic acid (50 mg, 26 umol) in thionyl chloride (3 mL) was added a drop of DMF at 25° C. and stirred at 78° C. for 1 hour. Then the mixture was concentrated in vacuo, the residue was then dissolved in THF (5 mL) and acetonitrile (5 mL) and cooled to 0° C. Diazomethyl(trimethyl)silane (2 M, 327 uL) was then added to the solution. The mixture was stirred at 0° C. for 0.5 hour and then HBr (64 mg, 785 umol) was added at the same temperature and stirred further for 1 h. The mixture was then concentrated in vacuo to give 2-bromo-1-(2,8-dimethylimidazo[1,2-b]pyridazin-6-yl)ethan-1-one (60 mg, crude) as colorless oil and used for the next step directly. MS: m/z 269.9 [M+2H]⁺

### Intermediate 6

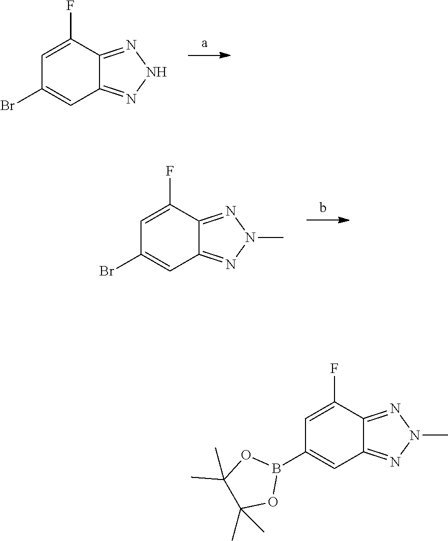

Step a: To a solution of 6-bromo-4-fluoro-2H-benzo[d][1,2,3]triazole (14.08 g, 65 mmol) in DMF (12 mL) was added NaH (1.88 g, 78 mmol, 60% in oil) and the mixture was stirred at 25° C. for 0.5 h, then Mel (10.2 g, 71.7 mmol) was added, and the reaction mixture was stirred for 12 h at the same temperature. The resulting mixture was concentrated under vacuum. The residue was purified using silica gel column chromatography (Hex:EtOAc=3:1) to afford 6-bromo-4-fluoro-2-methyl-2H-benzo[d][1,2,3]triazole as a yellow solid (3 g, 20% yield).

Step b: To a solution of 6-bromo-4-fluoro-2-methyl-2H-benzo[d][1,2,3]triazole (3 g, 13 mmol) in dioxane (50 mL) were added bis(pinacolato)diboron (6.6 g, 26 mmol) and potassium acetate (2.55 g, 26 mmol). The reaction mixture was degassed and backfilled with argon gas, and 1,1′-bis(diphenylphosphino)ferrocene palladium(II)dichloride dichloromethane (0.265 g, 0.32 mmol) was added. The reaction mixture was stirred at 100° C. for 10 hours. The resulting mixture was partitioned between EtOAc and water, the organic layer was concentrated under reduced pressure and purified on silica column with eluent (Hex:EtOAc=7:3) to get 1.6 g of 4-fluoro-2-methyl-6-(4,4,5,5-tetramethyl-1,3,2-dioxaborolan-2-yl)-2H-benzo[d][1,2,3]triazole. ¹H NMR (400 MHz, DMSO-d₆) δ 8.05 (s, 1H), 7.29 (d, J=11.1 Hz, 1H), 4.56 (s, 3H), 1.33 (s, 12H). M+=277

### Intermediate 7

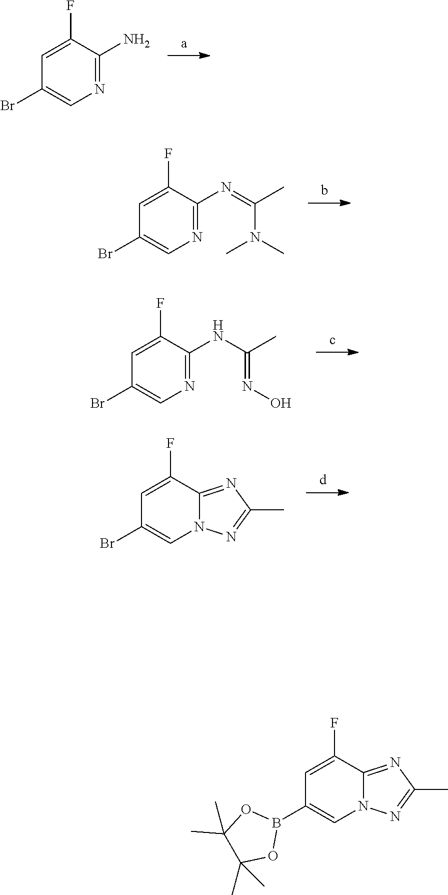

Step a: 5-Bromo-3-fluoropyridin-2-amine (28 g, 146.6 mmol) was dissolved in toluene (300 mL) and 1,1-dimethoxy-N,N-dimethylethanamine (35 mL, 234.5 mmol) was added. The reaction mixture was stirred overnight at reflux. After that the solvent was evaporated to afford 35 g of N′-(5-bromo-3-fluoropyridin-2-yl)-N,N-dimethylacetimidamide, which was used in the next step without further purifications.

Step b: Crude N′-(5-bromo-3-fluoropyridin-2-yl)-N,N-dimethylacetimidamide (35 g, 134.5 mmol) was dissolved in MeOH (300 mL) and hydroxylamine hydrochloride (1.4 g, 161.5 mmol) was added. The reaction mixture was stirred at RT overnight. After that, the solid was filtered off to afford N-(5-bromo-3-fluoropyridin-2-yl)-N′-hydroxyacetimidamide (15 g, 41% yield).

Step c: N-(5-bromo-3-fluoropyridin-2-yl)-N′-hydroxyacetimidamide (15 g, 60.5 mmol) was dissolved in THF (200 mL). The solution was cooled and 2,2,2-trifluoroacetic anhydride (17.1 mL, 120.9 mmol) was added dropwise. The reaction mixture was stirred at RT overnight. Then the solvent was evaporated in vacuo. The residue was dissolved in DCM/water mixture and neutralized with NaHCO₃ to pH=8-9. The organic layer was washed with brine, dried under Na₂SO₄ and evaporated under reduced pressure to afford 6-bromo-8-fluoro-2-methyl-[1,2,4]triazolo[1,5-a]pyridine (7 g, 50% of yield).

Step d: 6-Bromo-8-fluoro-2-methyl-[1,2,4]triazolo[1,5-a]pyridine (17) (7 g, 30.4 mmol), bis(pinacolato)diborane (8.1 g, 32 mmol) and potassium acetate (6 g, 60.8 mmol) were mixed in dioxane (50 mL). The resulting mixture was evacuated and backfilled with argon (three cycles), then Pd(dppf)2Cl₂ DCM (0.53 g, 1.52 mmol) was added under an argon atmosphere. The reaction mixture was stirred at 90° C. for 18 h under an argon atmosphere, then cooled, filtered through SiO₂, and concentrated under reduce pressure. The solid was dissolved in MTBE, stirred for 30 min and filtered off to give 8-fluoro-2-methyl-6-(4,4,5,5-tetramethyl-1,3,2-dioxaborolan-2-yl)-[1,2,4]triazolo[1,5-a]pyridine (5.5 g, 65% yield). ¹H NMR (400 MHz, DMSO-d₆) δ 8.74 (s, 1H), 7.48 (d, J=10.3 Hz, 1H), 2.54 (s, 3H), 1.33 (s, 12H). M+=277

### Intermediate 8

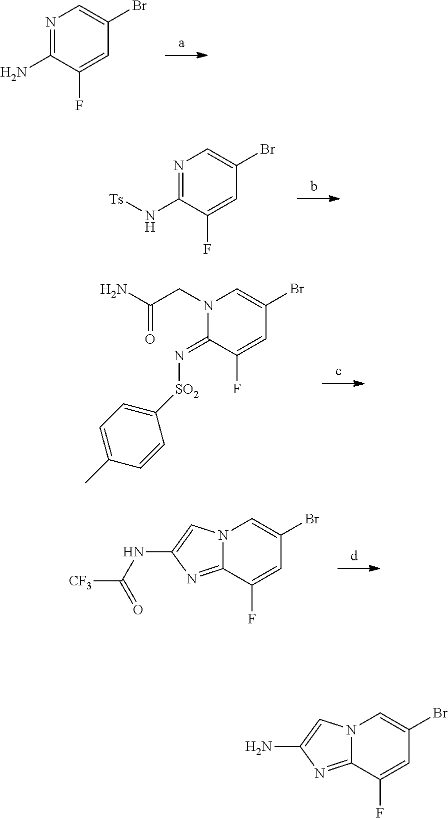 [SMILES: Nc1ncc(Br)cc1F ; Cc1ccc(S(=O)(=O)Nc2ncc(Br)cc2F)cc1 ; Cc1ccc(S(=O)(=O)/N=c2\c(F)cc(Br)cn2CC(N)=O)cc1 ; O=C(Nc1cn2cc(Br)cc(F)c2n1)C(F)(F)F ; Nc1cn2cc(Br)cc(F)c2n1]

Step a: To a mixture of 5-bromo-3-fluoropyridin-2-amine (5 g, 26.2 mmol) and pyridine (2.1 g, 26.2 mmol, 2.1 mL) was added 4-methylbenzene-1-sulfonyl chloride (5 g, 26.2 mmol) at 0° C., the reaction mixture was stirred at 100° C. for 12 h under a nitrogen atmosphere. The mixture was concentrated and then water (300 mL) was added and extracted with EtOAc (200 mL×2) and concentrated to give N-(5-bromo-3-fluoropyridin-2-yl)-4-methylbenzenesulfonamide (6.9 g, 19 mmol) as an off-white solid. MS: m/z 347.0 [M+2H]⁺

Step b: To a solution of N-(5-bromo-3-fluoro-2-pyridyl)-4-methyl-benzenesulfonamide (20 g, 57.94 mmol) and 2-iodoacetamide (10.72 g, 57.94 mmol) in DMF (100 mL) was added DIPEA (14.98 g, 115.88 mmol, 20.2 mL). The reaction mixture was stirred at 80° C. for 16 h. The mixture was concentrated and the crude material was purified by silica column chromatography (DCM/MeOH=10/1 to 6/1) to give 2-[(2E)-5-bromo-3-fluoro-2-(p-tolylsulfonylimino)-1-pyridyl]acetamide (6.5 g, 28% yield) as a white solid.

Step c: To a solution of 2-[(2E)-5-bromo-3-fluoro-2-(p-tolylsulfonylimino)-1-pyridyl]acetamide (6 g, 14.92 mmol) in DCM (80 mL) was added (2,2,2-trifluoroacetyl) 2,2,2-trifluoroacetate (18.80 g, 89.50 mmol, 12.4 mL) at 0° C., the reaction mixture was stirred at 25° C. for 12 h under N₂ atmosphere. The mixture was concentrated, and the crude material was purified by silica column chromatography (DCM/MeOH=20/1 to 10/1, to give N-(6-bromo-8-fluoro-imidazo[1,2-a]pyridin-2-yl)-2,2,2-trifluoro-acetamide (3 g, 61% yield) as an off-white solid.

Step d: To a solution of N-(6-bromo-8-fluoro-imidazo[1,2-a]pyridin-2-yl)-2,2,2-trifluoro-acetamide (3.2 g, 9.81 mmol) in THF (30 mL) and water (6 mL) at 20° C. was added NaOH (1.18 g, 29.44 mmol). The reaction mixture was stirred at 60° C. for 12 h under N₂ atmosphere. The mixture was concentrated. The residue was diluted with water (30 mL) and extracted with DCM (60 mL×3). The organic layer was washed with brine (100 mL), dried over Na₂SO₄, filtered and concentrated to give 6-bromo-8-fluoro-imidazo[1,2-a]pyridin-2-amine (2.0 g, 88% yield) as a pale yellow solid. MS: m/z 229.8 [M+H]⁺

### Intermediate 9

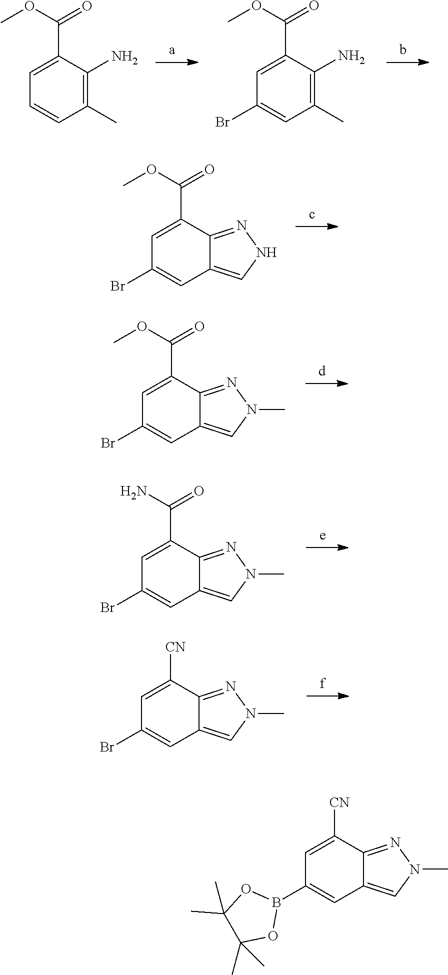

Step a: To a stirred solution of methyl 2-amino-3-methylbenzoate (48 g, 290.6 mmol) in acetonitrile (500 mL), N-bromosuccinimide (52 g, 290.6 mmol) was added in portions at rt. The resulting mixture was stirred at rt overnight. Acetonitrile was evaporated under reduce pressure. The residue was diluted with water (500 mL) and the product was extracted with DCM (3×200 mL). The combined organic layer was washed with water (3×150 mL), dried over Na₂SO₄, filtered, and evaporated under reduce pressure to give 67 g (95% yield) of methyl 5-bromo-2-methyl-2H-indazole-7-carboxylate.

Step b: To a stirred solution of methyl 2-amino-5-bromo-3-methylbenzoate (65 g, 266.3 mmol) in AcOH (3000 mL), sodium nitrite (21 g, 306.2 mmol) was added as a water solution. The resulting mixture was stirred overnight. The precipitate formed was filtered off. The mother liquid was concentrated under reduce pressure. The residue was diluted with water (400 mL) and extracted with EtOAc (3×150 mL). The combined organic layer was washed with water (3×150 mL), dried over Na₂SO₄, filtered, and evaporated under reduce pressure to give 34 g (50% yield) of methyl 5-bromo-2H-indazole-7-carboxylate.

Step c: To a stirred solution of methyl 5-bromo-2-methyl-2H-indazole-7-carboxylate (25 g, 98 mmol) in DCM (300 mL), triethyloxonium tetrafluoroborate (18.1 g, 122.5 mmol) was added in portions at 0° C. The reaction mixture was stirred at rt for 3 days. The resulting mixture was washed with water (3×100 mL). The organic layer was dried over Na₂SO₄, filtered, and evaporated in vacuo to give 19 g (73% yield) of methyl 5-bromo-2-methyl-2H-indazole-7-carboxylate.

Step d: To the stirred solution of methyl 5-bromo-2H-indazole-7-carboxylate (10 g, 37.2 mmol) in THF, KOH (2.7 g, 48.3 mmol) was added as a water solution. The reaction mixture was stirred for 2 h. The resulting mixture was concentrated under reduce pressure. The residue was diluted with water and acidified with NaHSO₄ to slightly acidic pH. The precipitate was filtered, dried to give 9 g of corresponding acid, which was dissolved in THF, and 1,1′-carbonyldiimidazole (8.6 g, 55.7 mmol) was added in portions. The resulting mixture was stirred for 4 h. Then gaseous ammonia was bubbled through the reaction mixture for 15 min. The obtained mixture was concentrated in vacuo. The solid residue was washed with water a few times and dried to give 8.4 g of 5-bromo-2-methyl-2H-indazole-7-carboxamide.

Step e: To a stirred mixture of 5-bromo-2-methyl-2H-indazole-7-carboxamide (8.4 g, 33 mmol) and pyridine (13 g, 165.3 mmol) in DCM (100 mL), TFAA (10.4 g, 49.5 mmol) was added dropwise. The resulting mixture was stirred for 4 h. The reaction mixture was concentrated under reduce pressure. The residue was diluted with DCM (100 mL) and washed with water (3×50 mL). The organic layer was dried over Na₂SO₄, filtered, and evaporated in vacuo to give 7.7 g (98% yield) of 5-bromo-2-methyl-2H-indazole-7-carbonitrile.

Step f: 5-Bromo-2-methyl-2H-indazole-7-carbonitrile (8) (7.8 g, 33 mmol), 4,4,5,5-tetramethyl-2-(4,4,5,5-tetramethyl-1,3,2-dioxaborolan-2-yl)-1,3,2-dioxaborolane (9.2 g, 36.3 mmol) and potassium acetate (6.5 g, 66 mmol) were mixed in dioxane (80 mL). The resulting mixture was evacuated and backfilled with argon (three cycles), then Pd(dppf)Cl₂·DCM (0.78 g, 3.3 mmol) was added under an argon atmosphere. The reaction mixture was stirred under an argon atmosphere at 90° C. for 15 h, then cooled and filtered. The filter cake was washed with 1,4-dioxane (2×20 mL) and discarded. The obtained mixture was concentrated under reduce pressure. The residue was dissolved in MTBE and filtered through short pad of SiO₂. MTBE was evaporated under reduced pressure. The residue was crystalized from MTBE/hexane to give 3.2 g of 2-methyl-5-(4,4,5,5-tetramethyl-1,3,2-dioxaborolan-2-yl)-2H-indazole-7-carbonitrile. ¹H NMR (400 MHz, DMSO-d₆) δ 8.68 (s, 1H), 8.48 (s, 1H), 7.91 (s, 1H), 4.26 (s, 3H), 1.32 (s, 12H). M+1=283

### Intermediate 10

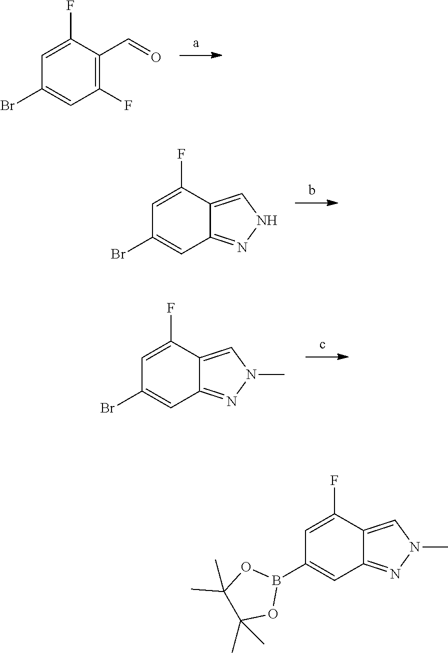

Step a: To a stirred solution of 4-bromo-2,6-difluorobenzaldehyde (50.0 g, 226.25 mmol, 226.25 mL) in THF (700 mL) was added hydrazine (48.33 g, 1.51 mol, 48.33 mL) at 0° C. The resulting mixture was allowed to warm to 25° C. and stirred at the same temperature for 16 h. The solvent was removed under reduced pressure. The residual solid was dissolved in DMSO (600 mL), then triethylamine (45.79 g, 452.49 mmol, 63.07 mL) was added, and the reaction mixture was stirred at 80° C. for 18 h. The obtained mixture was poured into water (1000 mL) and extracted with EtOAc (700 mL), the organic phase was separated, dried over Na₂SO₄, and evaporated to dryness to give 6-bromo-4-fluoro-1H-indazole.

Step b: To a mixture of 6-bromo-4-fluoro-1H-indazole (40.0 g, 186.03 mmol) in THF (700 mL) at 20° C. was added potassium tert-butoxide (31.31 g, 279.04 mmol). The mixture was stirred for 30 min at 20° C. before iodomethane (39.61 g, 279.04 mmol, 17.37 mL, 1.5 equiv) was added over 30 min. The mixture was stirred overnight at 60° C. As a result of taking an aliquot, complete conversion was obtained according to ¹H NMR data. Then the reaction mixture was poured onto water (1000 mL), extracted with EtOAc (1000 mL), the organic phase was dried over Na₂SO₄ and evaporated to dryness to give 56 g of yellow solid. The product was purified by FC (ISCO®: Interchim; 330 g SiO₂, Hex/EtOAc with EtOAc from 0-95%, flow rate=120 mL/min, R_{f}=5 CV) to give 6-bromo-4-fluoro-1-methyl-1H-indazole (20.0 g, 44% yield) as a yellow solid.

Step c: Potassium acetate (4.28 g, 43.66 mmol) and 4,4,5,5-tetramethyl-2-(4,4,5,5-tetramethyl-1,3,2-dioxaborolan-2-yl)-1,3,2-dioxaborolane (6.1 g, 24.01 mmol) were added to a solution of 6-bromo-4-fluoro-1-methyl-1H-indazole (5.0 g, 21.83 mmol) in dry dioxane (700 mL). The solution was degassed with argon gas for 30 minutes. Then d(dppf)Cl₂·CH₂Cl₂ (1.78 g, 2.18 mmol) was added, and the resulting solution was stirred under reflux for 12 hours. As a result of taking an aliquot, complete conversion was obtained according to ¹H NMR data. Then the reaction mixture was evaporated to dryness, poured onto water (350 mL), and extracted with EtOAc (450 mL), the organic phase was dried over Na₂SO₄ and evaporated to dryness. The product was purified by FC (ISCO®: Interchim; 120 g SiO₂, Hex/EtOAc with EtOAc from 0-95%, flow rate=120 mL/min) to give 4-fluoro-1-methyl-6-(4,4,5,5-tetramethyl-1,3,2-dioxaborolan-2-yl)-1H-indazole (1.02 g, 10% yield) as a yellow solid. ¹H NMR (400 MHz, DMSO-d₆) δ 8.54 (s, 1H), 7.78 (s, 1H), 6.86 (d, J=10.9 Hz, 1H), 4.20 (s, 3H), 1.30 (s, 12H). M+=277

### Intermediate 11

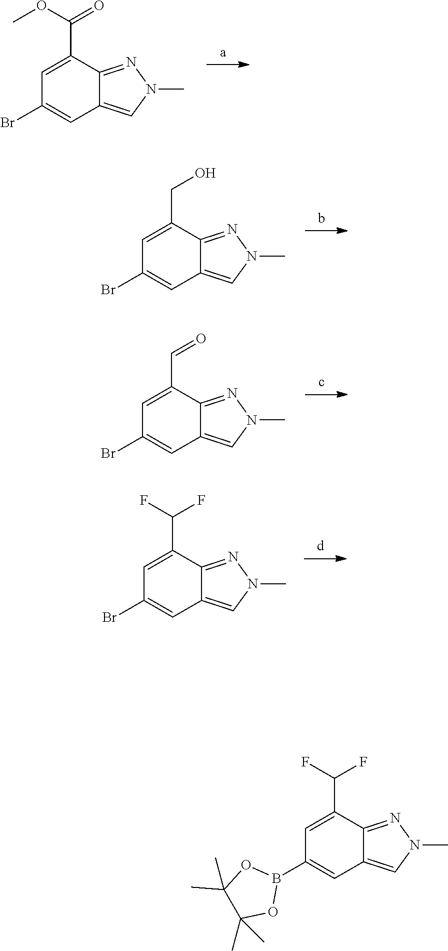

Step a: To the previously prepared suspension of LiAlH₄ (0.68 g, 17.8 mmol) in THF (50 mL) under an argon atmosphere, a solution of methyl 5-bromo-2-methyl-2H-indazole-7-carboxylate (4 g, 14.9 mmol) was added dropwise at 0° C. The resulting mixture was stirred at rt for 1.5 h, then water (5 mL) was added dropwise as 20% solution in THF. The reaction mixture was filtered; the mother liquid was concentrated under reduce pressure to give 2.6 g of 5-bromo-2-methyl-2H-indazol-7-yl)methanol.

Step b: To a stirred solution of 5-bromo-2-methyl-2H-indazol-7-yl)methanol (2.6 g, 10.8 mmol) in THF (50 mL), MnO₂ (4.7 g, 53.9 mmol) was added. The resulting mixture was stirred at 70° C. for 2 days. The reaction mixture was filtered; the mother liquid was concentrated in vacuo to give 2.2 g of 5-bromo-2-methyl-2H-indazole-7-carbaldehyde.

Step c: To a stirred solution of 5-bromo-2-methyl-2H-indazole-7-carbaldehyde (2.2 g, 9.2 mmol) in DCM (40 mL), diethylaminosulfur trifluoride (DAST, 5 g, 27.6 mmol) was added dropwise. The resulting mixture was stirred overnight. The reaction mixture was quenched with a Na₂CO₃ solution. The organic layer was washed with water (2×30 mL), dried over Na₂SO₄, filtered, and evaporated in vacuo to give 2.5 g of 5-bromo-7-(difluoromethyl)-2-methyl-2H-indazole.

Step d: 5-Bromo-7-(difluoromethyl)-2-methyl-2H-indazole (2.5 g, 9.6 mmol), 4,4,5,5-tetramethyl-2-(4,4,5,5-tetramethyl-1,3,2-dioxaborolan-2-yl)-1,3,2-dioxaborolane (2.67 g, 10.5 mmol) and potassium acetate (1.88 g, 19.2 mmol) were mixed in dioxane (40 mL). The resulting mixture was evacuated and backfilled with argon (three cycles), then Pd(dppf)Cl₂·DCM (0.25 g, 9.6 mmol) was added under an argon atmosphere. The reaction mixture was stirred under an argon atmosphere at 90° C. for 15 h, then cooled and filtered. The filter cake was washed with 1,4-dioxane (2×20 mL) and discarded. The obtained mixture was concentrated under reduce pressure. The residue was dissolved in MTBE and filtered through short pad of SiO₂. MTBE was evaporated under reduced pressure. The residue was crystalized from MTBE/hexane to give 1.17 g of 7-(difluoromethyl)-2-methyl-5-(4,4,5,5-tetramethyl-1,3,2-dioxaborolan-2-yl)-2H-indazole. ¹H NMR (400 MHz, Chloroform-d) δ 8.34 (s, 1H), 8.02 (s, 1H), 7.89 (s, 1H), 7.32-6.98 (m, 2H), 4.29 (s, 3H), 1.38 (s, 12H). M+=227

### Intermediate 12

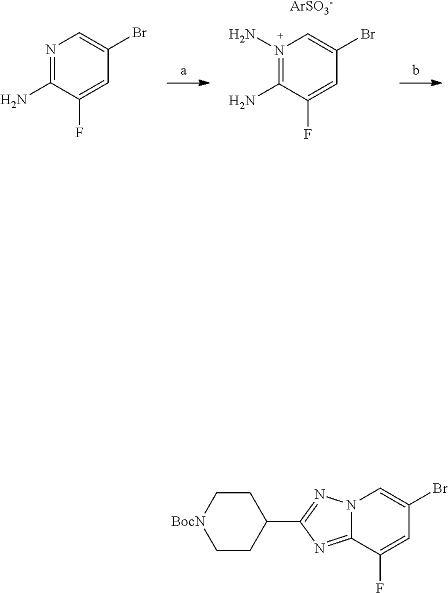

Step a: To a solution of 5-bromo-3-fluoropyridin-2-amine (5 g, 26 mmol) in DCM (50 mL), cooled with an ice bath, was added dropwise a solution of (O-(mesitylsulfonyl)hydroxylamine) (6.2 g, 29 mmol) in DCM (100 mL). The reaction mixture was stirred overnight at rt. The precipitated solid was collected and dried to afford 6.2 g of crude desired compound (58% yield) which was carried forward as is.

Step b: To a solution of the salt obtained from the previous step (6.2 g, 15 mmol) and KOH (1.28 g, 23 mmol) in methanol (100 mL) was added tert-butyl 4-formylpiperidine-1-carboxylate (3.2 g, 15 mmol) and the reaction mixture was stirred overnight at rt. The resulting mixture was evaporated under reduced pressure, the residue was diluted with EtOAc and washed with water and brine. The organic layer was dried over Na₂SO₄ and evaporated under reduced pressure to obtain 3.8 g of tert-butyl 4-(6-bromo-8-fluoro-[1,2,4]triazolo[1,5-a]pyridin-2-yl)piperidine-1-carboxylate (62% yield) which was used crude. 1H NMR (400 MHz, DMSO-d6) δ 9.25 (d, J=1.5 Hz, 1H), 7.95 (dd, J=9.9, 1.6 Hz, 1H), 3.96 (d, J=13.1 Hz, 2H), 3.11 (tt, J=11.6, 4.0 Hz, 1H), 2.96 (bs, 2H), 2.01 (dd, J=13.7, 3.7 Hz, 2H), 1.73-1.58 (m, 2H), 1.41 (s, 9H). LCMS: 1.47 min, 298.2 [M-tBu]⁺

### Intermediate 13

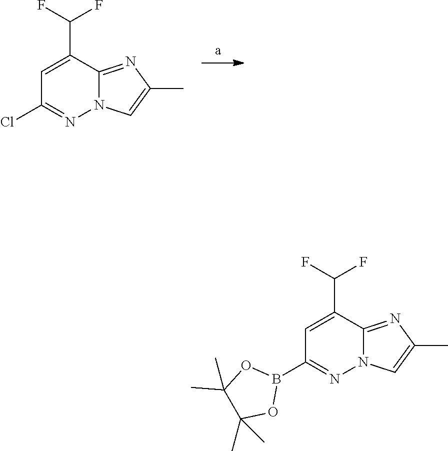

Step a: To a solution of 6-chloro-8-(difluoromethyl)-2-methyl-imidazo[1,2-b]pyridazine (50 mg, 229.78 μmol, 1.0 eq.) and 4,4,5,5-tetramethyl-2-(4,4,5,5-tetramethyl-1,3,2-dioxaborolan-2-yl)-1,3,2-dioxaborolane (70.02 mg, 275.73 μmol, 1.2 eq.) in Dioxane (3 mL, 0.077M) was added cyclopentyl(diphenyl)phosphane; dichloropalladium; iron (16.81 mg, 22.98 μmol, 0.1 eq.) and KOAc (45.10 mg, 459.55 μmol, 2.0 eq.). The mixture was stirred under nitrogen at 90° C. for 2 hours. The material was then filtered, the filtrate concentrated, and the concentrated was used for the next step directly without further purification. MS: m/z 365.1 [M+3MeO-2F]⁺; RT: 1.78 min (Method 10)

### Intermediate 14

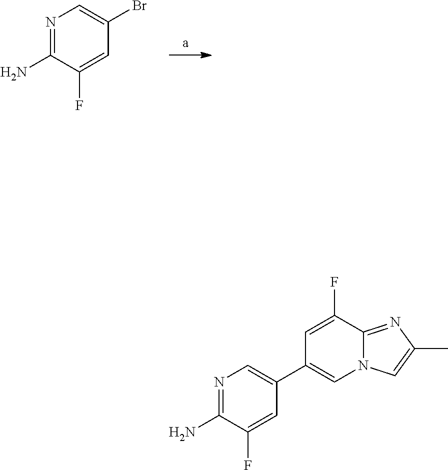 [SMILES: Nc1ncc(Br)cc1F ; Cc1cn2cc(-c3cnc(N)c(F)c3)cc(F)c2n1]

To a solution of 5-bromo-3-fluoro-pyridin-2-amine (964 mg, 3.49 mmol) and (2,8-dimethylimidazo[1,2-b]pyridazin-6-yl)boronic acid (1 g, 5.24 mmol) in dioxane (12 mL) and water (4 mL) was added Cs₂CO₃(3.41 g, 10.47 mmol) and Pd(dppf)Cl₂ (255 mg, 0.35 mmol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified by flash silica gel chromatography (0% to 100% EtOAc:PE) to give 3-fluoro-5-(8-fluoro-2-methyl-imidazo[1,2-a]pyridin-6-yl)pyridin-2-amine (312 mg, 1.2 mmol, 34% yield) as a white fluffy solid. MS: m/z 261.1 [M+H]⁺, ¹H NMR (400 MHz, DMSO-d6) δ ppm: 8.68 (s, 1H), 8.16 (s, 1H), 7.85-7.67 (m, 2H), 7.48 (d, J=13.0 Hz, 1H), 6.44 (s, 2H), 2.36 (s, 3H).

### Section 3. Synthetic Processes to Prepare Compounds of the Disclosure

### Example 1—Compound 64

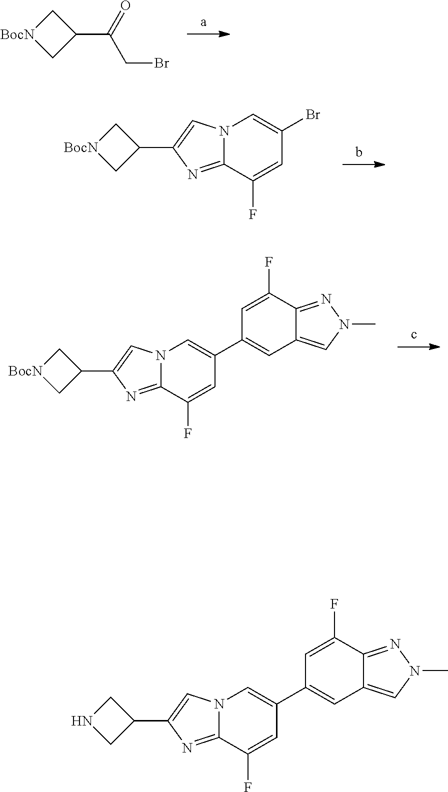 [SMILES: CC(C)(C)OC(=O)N1CC(C(=O)CBr)C1 ; CC(C)(C)OC(=O)N1CC(c2cn3cc(Br)cc(F)c3n2)C1 ; Cn1cc2cc(-c3cc(F)c4nc(C5CN(C(=O)OC(C)(C)C)C5)cn4c3)cc(F)c2n1 ; Cn1cc2cc(-c3cc(F)c4nc(C5CNC5)cn4c3)cc(F)c2n1]

Step a: Tert-butyl 3-(2-bromoacetyl)azetidine-1-carboxylate (455 mg, 1.64 mmol 1.25 eq.) and 5-bromo-3-fluoro-pyridin-2-amine (250 mg, 1.31 mmol, 1.0 eq.) were dissolved in water (6.5 mL, 0.2 M) then heated to 80° C. for 16 hours. The solution was then concentrated before being dry loaded onto normal phase silica column and purified via 0-100% EtOAc:heptane over 7 minutes followed by 0-25% MeOH:DCM over 5 minutes. Product elutes at 1% MeOH. Isolated tert-butyl 3-(6-bromo-8-fluoro-imidazo[1,2-a]pyridin-2-yl)azetidine-1-carboxylate (227.5 mg, 24% yield). MS: m/z 313.9 [M+H]⁺.

Step b: Tert-butyl 3-(6-bromo-8-fluoro-imidazo[1,2-a]pyridin-2-yl)azetidine-1-carboxylate (56.9 mg, 78.38 μmol, 1.0 eq.) and 7-fluoro-2-methyl-5-(4,4,5,5-tetramethyl-1,3,2-dioxaborolan-2-yl)-2H-indazole (29.40 mg, 94.06 mmol, 1.2 eq.) were dissolved in water (1 mL, 0.078 M) and dioxane (1 mL, 0.078 M) before cesium carbonate (51.08 mg, 156.7 μmol, 2 eq.) and Pd(dppf)2Cl DCM adduct (6.40 mg, 7.84 μmol, 0.1 eq.) were added. The solution was then degassed with nitrogen before being heated to 100° C. for 16 hours. The solution was then concentrated before tert-butyl 3-(8-fluoro-6-(7-fluoro-2-methyl-2H-indazol-5-yl)imidazo[1,2-a]pyridin-2-yl)azetidine-1-carboxylate was telescoped to deprotection crude. MS: m/z 440.2 [M+H]⁺

Step c: Tert-butyl 3-(8-fluoro-6-(7-fluoro-2-methyl-2H-indazol-5-yl)imidazo[1,2-a]pyridin-2-yl)azetidine-1-carboxylate (34.44 mg, 78.38 μmol, 1.0 eq) was dissolved in DCM (1 mL, 0.078 M) before 4 M HCl in dioxane (196 μL, 10 eq.) was added. The solution was then stirred at 40° C. for 16 hours before being concentrated and submitted to HPLC purification (Column: Sunfire C18 100×19 mm, 5 mm; Mobile phase A: MeCN; Mobile phase B: H₂O; Modifier: 0.1% TFA) to obtain to obtain 5-(2-(azetidin-3-yl)-8-fluoroimidazo[1,2-a]pyridin-6-y)-7-fluoro-2-methyl-2H-indazole (2.5 mg, 7.05%). MS: m/z 340.2 [M+H]+; RT: 0.99 m (Method 4); NMR: ¹H NMR (600 MHz, DMSO-d6) δ ppm 4.21-4.34 (m, 9H) 7.45-7.51 (m, 1H) 7.70-7.75 (m, 1H) 7.91-7.95 (m, 1H) 7.98-8.02 (m, 1H) 8.54-8.58 (m, 1H) 8.88-8.91 (m, 1H).

Using the procedure described for Example 1 above, additional compounds described herein were prepared by substituting the appropriate bromoketone starting material in step a, the appropriate boronic ester or acid equivalent in step b, suitable reagents and reaction conditions, obtaining compounds such as those selected from:

| Cpd ID | Data |
|---|---|
| 13 | Starting materials: tert-butyl 3-(3-bromo-2-oxopropyl)azetidine-1- |
| | carboxylate and 2,8-dimethyl-6-(4,4,5,5-tetramethyl-1,3,2-dioxaborolan-2- |
| | yl)imidazo[1,2-b]pyridazine |
| | MS: m/z 351.1 [M + H]⁺; RT: 0.37 min (Method 4) |
| | ¹H NMR (600 MHz, DMSO-d6) δ ppm 2.41-2.42 (m, 3 H) 2.60-2.62 (m, |
| | 3 H) 3.05-3.08 (m, 2 H) 3.17-3.26 (m, 1 H) 3.83-3.88 (m, 2 H) 4.03-4.06 |
| | (m, 2 H) 7.64-7.68 (m, 1 H) 7.74-7.79 (m, 1 H) 7.98-8.00 (m, 1 H) 8.05- |
| | 8.09 (m, 1 H) 8.50-8.58 (m, 1 H) 8.63-8.73 (m, 1 H) 9.19-9.22 (m, 1 H) |
| 12 | Starting materials: tert-butyl (4-bromo-3-oxobutyl)(methyl)carbamate and |
| | 2,8-dimethyl-6-(4,4,5,5-tetramethyl-1,3,2-dioxaborolan-2-yl)imidazo[1,2- |
| | b]pyridazine |
| | MS: m/z 339.3 [M + H]⁺; RT: 0.76 min (Method 4) |
| | ¹H NMR (600 MHz, DMSO-d6) δ ppm 2.39-2.43 (m, 4 H) 2.52-2.55 (m, |
| | 6 H) 2.59-2.62 (m, 3 H) 3.00-3.06 (m, 1 H) 7.62-7.67 (m, 1 H) 7.73-7.79 |
| | (m, 1 H) 7.96-8.02 (m, 1 H) 8.04-8.08 (m, 1 H) 9.18-9.24 (m, 1 H) |
| 15 | Starting materials: tert-butyl 3-(3-bromo-2-oxopropyl)piperidine-1- |
| | carboxylate and 8-fluoro-2-methyl-6-(4,4,5,5-tetramethyl-1,3,2- |
| | dioxaborolan-2-yl)imidazo[1,2-a]pyridine |
| | MS: m/z 382.2 [M + H]/2⁺; RT: 0.33 min (Method 4) |
| | ¹H NMR (600 MHz, DMSO-d6) δ ppm 1.32-1.41 (m, 2 H) 1.76-1.83 (m, |
| | 2 H) 1.99-2.07 (m, 1 H) 2.37-2.40 (m, 3 H) 2.67-2.72 (m, 2 H) 2.81-2.88 |
| | (m, 2 H) 2.88-2.90 (m, 1 H) 3.10-3.26 (m, 1 H) 7.53-7.61 (m, 2 H) 7.81 |
| | 7.83 (m, 1 H) 7.85-7.88 (m, 1 H) 8.79-8.86 (m, 2 H) |
| 21 | Starting materials: tert-butyl 3-(2-bromoacetyl)azetidine-1-carboxylate and |
| | 8-fluoro-2-methyl-6-(4,4,5,5-tetramethyl-1,3,2-dioxaborolan-2- |
| | yl)imidazo[1,2-a]pyridine |
| | MS: m/z 354.1 [M + H]/2+; RT: 0.33 min (Method 4) |
| | ¹H NMR (600 MHz, DMSO-d6) δ ppm 3.03-3.07 (m, 2 H) 3.17-3.25 (m, |
| | 1 H) 3.82-3.86 (m, 2 H) 4.03-4.08 (m, 2 H) 4.18-4.21 (m, 3 H) 7.58-7.60 |
| | (m, 1 H) 7.69-7.72 (m, 1 H) 7.88-7.91 (m, 1 H) 8.03-8.05 (m, 1 H) 8.42- |
| | 8.44 (m, 1 H) 8.48-8.54 (m, 1 H) 8.61-8.70 (m, 1 H) 8.76-8.79 (m, 1 H) |
| 24 | Starting materials: tert-butyl (4-bromo-3-oxobutyl)(methyl)carbamate and |
| | 8-fluoro-2-methyl-6-(4,4,5,5-tetramethyl-1,3,2-dioxaborolan-2- |
| | yl)imidazo[1,2-a]pyridine |
| | MS: m/z 342.3 [M + H]⁺; RT: 0.70 min (Method 4) |
| | ¹H NMR (600 MHz, DMSO-d6) δ ppm 2.37-2.39 (m, 3 H) 2.61-2.66 (m, |
| | 4 H) 3.06-3.13 (m, 3 H) 7.54-7.59 (m, 1 H) 7.62-7.67 (m, 1 H) 7.81-7.85 |
| | (m, 1 H) 7.93-7.97 (m, 1 H) 8.42-8.51 (m, 2 H) 8.81-8.85 (m, 1 H) 8.88- |
| | 8.90 (m, 1 H) |
| 14 | Starting materials: tert-butyl 3-(3-bromo-2-oxopropyl)piperidine-1- |
| | carboxylate and |
| | 8-fluoro-2-methyl-6-(4,4,5,5-tetramethyl-1,3,2-dioxaborolan-2- |
| | yl)imidazo[1,2-a]pyridine |
| | MS: m/z 382.3 [M + H]⁺; RT: 0.96 min (Method 4) |
| | ¹H NMR (600 MHz, DMSO-d6) δ ppm 1.35-1.44 (m, 2 H) 1.78-1.85 (m, |
| | 2 H) 2.00-2.09 (m, 1 H) 2.67-2.72 (m, 2 H) 2.81-2.91 (m, 2 H) 3.05-3.14 |
| | (m, 1 H) 3.22-3.29 (m, 1 H) 4.21-4.25 (m, 3 H) 7.44-7.48 (m, 1 H) 7.59- |
| | 7.63 (m, 1 H) 7.84-7.87 (m, 1 H) 7.90-7.93 (m, 1 H) 8.11-8.22 (m, 1 H) |
| | 8.47-8.53 (m, 1 H) 8.54-8.58 (m, 1 H) 8.80-8.85 (m, 1 H) |
| 23 | Starting materials: tert-butyl 3-(2-bromoacetyl)azetidine-1-carboxylate and |
| | 7-fluoro-2-methyl-5-(4,4,5,5-tetramethyl-1,3,2-dioxaborolan-2-yl)-2H- |
| | indazole |
| | MS: m/z 340.3 [M + H]⁺; RT: 0.98 min (Method 4) |
| | ¹H NMR (600 MHz, DMSO-d6) δ ppm 4.21-4.34 (m, 9 H) 7.45-7.51 (m, |
| | 1 H) 7.70-7.75 (m, 1 H) 7.91-7.95 (m, 1 H) 7.98-8.02 (m, 1 H) 8.54-8.58 |
| | (m, 1 H) 8.88-8.91 (m, 1 H) |
| 19 | Starting materials: tert-butyl 3-(2-bromoacetyl)azetidine-1-carboxylate and |
| | 7-fluoro-2-methyl-5-(4,4,5,5-tetramethyl-1,3,2-dioxaborolan-2-yl)-2H- |
| | indazole |
| | MS: m/z 354.3 [M + H]⁺; RT: 1.51 min (Method 2) |
| | ¹H NMR (600 MHz, DMSO-d6) δ ppm 3.03-3.07 (m, 2 H) 3.23 (br s, 1 H) |
| | 3.82-3.86 (m, 2 H) 4.01-4.07 (m, 2 H) 4.21-4.24 (m, 3 H) 7.45-7.49 (m, |
| | 1 H) 7.62-7.66 (m, 1 H) 7.86-7.90 (m, 1 H) 7.90-7.93 (m, 1 H) 8.48-8.54 |
| | (m, 1 H) 8.54-8.58 (m, 1 H) 8.62-8.69 (m, 1 H) 8.82-8.84 (m, 1 H) |
| 18 | Starting materials: tert-butyl (4-bromo-3-oxobutyl)(methyl)carbamate and |
| | 7-fluoro-2-methyl-5-(4,4,5,5-tetramethyl-1,3,2-dioxaborolan-2-yl)-2H- |
| | indazole |
| | MS: m/z 342.1 [M + H]⁺; RT: 0.44 min (Method 4) |
| | ¹H NMR (400 MHz, METHANOL-d4) δ ppm 2.77-2.81 (m, 3 H) 3.18- |
| | 3.25 (m, 2 H) 3.42-3.47 (m, 2 H) 4.22-4.27 (m, 3 H) 7.60-7.65 (m, 2 H) |
| | 7.70-7.75 (m, 1 H) 7.95-7.98 (m, 1 H) 8.00-8.02 (m, 1 H) 8.29-8.31 (m, |
| | 1 H) 8.62-8.64 (m, 1 H) |
| 2 | Starting materials: tert-butyl 4-(2-bromoacetyl)piperidine-1-carboxylate and |
| | (2-methylimidazo[1,2-b]pyridazin-6-yl)boronic acid |
| | MS: m/z 351.3 [M + H]⁺; RT: 0.71 min (Method 4) |
| 17 | Starting materials: tert-butyl 3-(3-bromo-2-oxopropyl)piperidine-1- |
| | carboxylate and |
| | (2-methylimidazo[1,2-b]pyridazin-6-yl)boronic acid |
| | MS: m/z 364.3 [M + H]⁺; RT: 0.90 min (Method 4) |
| | ¹H NMR (600 MHz, DMSO-d6) δ ppm 1.34-1.44 (m, 2 H) 1.79-1.86 (m, |
| | 2 H) 1.99-2.07 (m, 1 H) 2.66-2.73 (m, 2 H) 2.83-2.93 (m, 2 H) 3.07-3.13 |
| | (m, 1 H) 3.23-3.28 (m, 1 H) 4.19 (s, 3 H) 7.54-7.61 (m, 2 H) 7.69-7.72 |
| | (m, 1 H) 7.85-7.88 (m, 1 H) 8.02-8.06 (m, 1 H) 8.14-8.22 (m, 1 H) 8.41- |
| | 8.44 (m, 1 H) 8.46-8.52 (m, 1 H) 8.76-8.79 (m, 1 H) |
| 16 | Starting materials: tert-butyl 3-(2-bromoacetyl)piperidine-1-carboxylate and |
| | (2-methylimidazo[1,2-b]pyridazin-6-yl)boronic acid |
| | MS: m/z 350.3 [M + H]⁺; RT: 0.93 min (Method 4) |
| | ¹H NMR (600 MHz, DMSO-d6) δ ppm 1.57-1.69 (m, 2 H) 1.73-1.78 (m, |
| | 1 H) 2.02-2.09 (m, 1 H) 2.68-2.74 (m, 1 H) 2.80-2.88 (m, 1 H) 2.94-3.00 |
| | (m, 1 H) 3.09 (br d, J = 12.35 Hz, 1 H) 3.34-3.39 (m, 2 H) 4.14-4.18 (m, 3 |
| | H) 7.52-7.57 (m, 2 H) 7.65-7.68 (m, 1 H) 7.84-7.87 (m, 1 H) 7.98-8.01 |
| | (m, 1 H) 8.38-8.40 (m, 1 H) 8.71-8.74 (m, 1 H) |
| 20 | Starting materials: tert-butyl 3-(3-bromo-2-oxopropyl)azetidine-1- |
| | carboxylate and (2-methylimidazo[1,2-b]pyridazin-6-yl)boronic acid |
| | MS: m/z 336.3 [M + H]⁺; RT: 1.37 min (Method 2) |
| | ¹H NMR (600 MHz, DMSO-d6) δ ppm 3.04-3.06 (m, 2 H) 3.17-3.25 (m, |
| | 1 H) 3.82-3.86 (m, 2 H) 4.02-4.07 (m, 2 H) 4.19-4.21 (m, 3 H) 7.57-7.62 |
| | (m, 2 H) 7.69-7.73 (m, 1 H) 7.87-7.90 (m, 1 H) 8.02-8.05 (m, 1 H) 8.41- |
| | 8.44 (m, 1 H) 8.48-8.56 (m, 1 H) 8.62-8.71 (m, 1 H) 8.76-8.79 (m, 1 H) |
| 22 | Starting materials: tert-butyl (4-bromo-3-oxobutyl)(methyl)carbamate and |
| | (2-methylimidazo[1,2-b]pyridazin-6-yl)boronic acid |
| | MS: m/z 324.3 [M + H]+; RT: 0.86 min (Method 4) |
| | ¹H NMR (400 MHz, METHANOL-d4) δ ppm 2.77-2.81 (m, 3 H) 3.18- |
| | 3.25 (m, 2 H) 3.42-3.47 (m, 2 H) 4.22-4.27 (m, 3 H) 7.60-7.65 (m, 2 H) |
| | 7.70-7.75 (m, 1 H) 7.95-7.98 (m, 1 H) 8.00-8.02 (m, 1 H) 8.29-8.31 (m, |
| | 1 H) 8.62-8.64 (m, 1 H) |
| 65 | Starting materials: tert-butyl 3-(2-bromoacetyl)pyrrolidine-1-carboxylate |
| | and |
| | (2-methylimidazo[1,2-b]pyridazin-6-yl)boronic acid |
| | MS: m/z 336.2 [M + H]⁺; RT: 0.85 min (Method 4) |
| | ¹H NMR (500 MHz, DMSO-d6) δ ppm 1.85-1.96 (m, 1 H) 2.08-2.16 (m, |
| | 1 H) 2.83-3.04 (m, 3 H) 3.16-3.26 (m, 1 H) 3.31-3.39 (m, 1 H) 4.19 (s, 3 |
| | H) 5.17 (tt, J = 13.68, 6.88 Hz, 1 H) 7.56-7.60 (m, 2 H) 7.68-7.71 (m, 1 H) |
| | 7.85-7.89 (m, 1 H) 8.02-8.07 (m, 1 H) 8.41-8.45 (m, 1 H) 8.72-8.75 (m, |
| | 1 H) |
| 68 | Starting materials: tert-butyl (1-(2-bromoacetyl)-2-oxabicyclo[2.1.1]hexan- |
| | 4-yl)carbamate and 7-fluoro-2-methyl-5-(4,4,5,5-tetramethyl-1,3,2- |
| | dioxaborolan-2-yl)-2H-indazole |
| | MS: m/z 382.3 [M + H]⁺; RT: 1.45 min (Method 7) |
| 67 | Starting materials: tert-butyl 4-(2-bromoacetyl)-2-azabicyclo[2.1.1]hexane- |
| | 2-carboxylate and 7-fluoro-2-methyl-5-(4,4,5,5-tetramethyl-1,3,2- |
| | dioxaborolan-2-yl)-2H-indazole |
| | MS: m/z 366.2 [M + H]⁺; ¹H NMR: (500 MHz, METHANOL-d4) □ ppm = |
| | 8.59 (d, J = 1.37 Hz, 1H), 8.38 (d, J = 2.59 Hz, 1H), 7.92 (d, J = 2.90 Hz, |
| | 1H), 7.83 (d, J = 1.22 Hz, 1H), 7.52 (dd, J = 12.05, 1.37 Hz, IH), 7.42-7.33 |
| | (m, 1 H), 7.37 (dd, J = 12.66, 1.37 Hz, 1H), 4.27 (s, 3 H), 3.89 (t, J = 1.68 |
| | Hz, 1H), 3.30-3.28 (m, 2H), 2.33-2.24 (m, 2H), 1.83-1.73 (m, 2H). |
| 63 | Starting materials: tert-butyl 4-(2-bromoacetyl)-2,2-dimethylpiperidine-1- |
| | carboxylate and 7-fluoro-2-methyl-5-(4,4,5,5-tetramethyl-1,3,2- |
| | dioxaborolan-2-yl)-2H-indazole |
| | MS: m/z 396.3 [M + H]⁺; RT: 0.44 min (Method 4) |
| 60 | Starting materials: tert-butyl 4-(2-bromoacetyl)-2-methylpiperidine-1- |
| | carboxylate and 2,8-dimethyl-6-(4,4,5,5-tetramethyl-1,3,2-dioxaborolan-2- |
| | yl)imidazo[1,2-b]pyridazine |
| | MS: 379.3 [M + H]⁺; RT: 1.81 min (Method 1) |
| | ¹H NMR (500 MHz, DMSO-d6) δ ppm 1.15-1.20 (m, 3 H) 1.21-1.35 (m, |
| | 6 H) 1.60-1.70 (m, 1 H) 1.75-1.85 (m, 1 H) 2.14-2.26 (m, 2 H) 3.07-3.16 |
| | (m, 3 H) 3.58-3.69 (m, 1 H) 7.68 (s, 1 H) 7.75-7.81 (m, 1 H) 7.98 (d, J = 3.32 |
| | Hz, 1 H) 8.07-8.11 (m, 1 H) 8.26-8.34 (m, 1 H) 8.71-8.81 (m, 1 H) 9.17- |
| | 9.22 (m, 1 H) Note: 1 extra proton from quaternary amine salt |
| 62 | Starting materials:: 2-bromo-1-(2,2-dimethylpiperidin-4-yl)ethan-1-one and |
| | 2-methyl-6-(4,4,5,5-tetramethyl-1,3,2-dioxaborolan-2-yl)imidazo[1,2- |
| | b]pyridazine |
| | MS: 379.3 [M + H]+; RT: 0.81 min (Method 4) |
| | ¹H NMR (500 MHz, DMSO-d6) δ ppm 1.37 (s, 3 H) 1.42 (s, 3 H) 2.04-2.18 |
| | (m, 2 H) 2.24-2.50 (m, 3 H) 3.07-3.15 (m, 2 H) 3.22-3.34 (m, 2 H) 3.56- |
| | 3.69 (m, 1 H) 7.75-7.83 (m, 2 H) 7.97-8.01 (m, 1 H) 8.11-8.14 (m, 1 H) |
| | 8.15-8.19 (m, 1 H) 8.39-8.48 (m, 1 H) 8.67-8.75 (m, 1 H) 9.21-9.25 (m, |
| | 1 H) |
| 61 | Starting materials: tert-butyl 4-(2-bromoacetyl)-2-methylpiperidine-1- |
| | carboxylate and 2-methyl-6-(4,4,5,5-tetramethyl-1,3,2-dioxaborolan-2- |
| | yl)imidazo[1,2-b]pyridazine |
| | MS: 365.3 [M + H]⁺; RT: 0.77 min (Method 3) |
| | ¹H NMR (500 MHz, DMSO-d6) δ ppm 1.13-1.18 (m, 3 H) 1.27-1.30 (m, |
| | 3 H) 1.59-1.69 (m, 1 H) 1.77-1.86 (m, 1 H) 2.15-2.27 (m, 2 H) 3.07-3.14 |
| | (m, 3 H) 3.58-3.66 (m, 1 H) 7.75-7.82 (m, 2 H) 7.98-8.00 (m, 1 H) 8.12- |
| | 8.15 (m, 1 H) 8.16-8.20 (m, 1 H) 8.26-8.32 (m, 1 H) 8.71-8.78 (m, 1 H) |
| | 9.21-9.25 (m, 1 H) Note: 1 extra proton from quaternary amine salt |
| 169 | Starting materials: 2-bromo-1-(tetrahydro-2H-pyran-4-yl)ethan-1-one |
| | MS: m/z 366.1 [M + H]⁺; RT: 0.93 min (Method 10) |
| 215 | Starting materials: tert-butyl (2S,4r,6R)-4-(2-bromoacetyl)-2,6- |
| | dimethylpiperidine-1-carboxylate |
| | MS: m/z 393.2 [M + H]⁺; RT: 1.597 min (Method 10) |

### Example 2—Compound 66

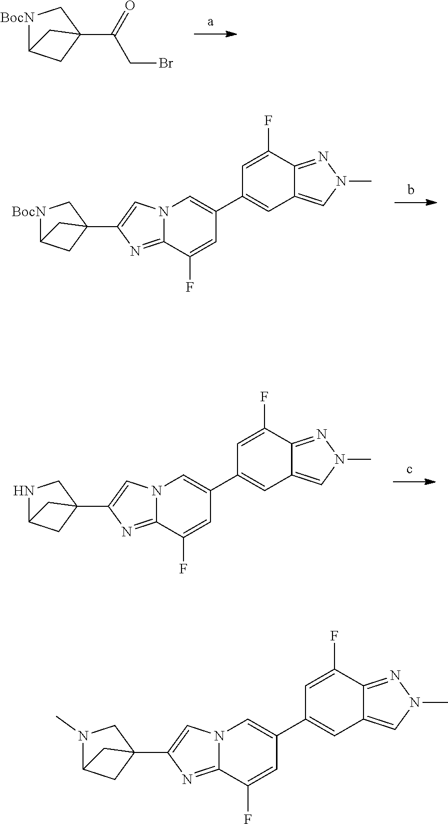

Step a: To a solution of tert-butyl 4-(2-bromoacetyl)-2-azabicyclo[2.1.1]hexane-2-carboxylate (70 mg, 230 μmol) in t-BuOH (5 mL) was added 3-fluoro-5-(7-fluoro-2-methyl-indazol-5-yl)pyridin-2-amine (50 mg, 192 μmol) and NaHCO₃ (32 mg, 384 μmol) and the mixture was stirred at 70° C. for 16 h. The mixture was concentrated in vacuo to give the residue, which was purified by flash silica gel chromatography (PE/EA=1/1) to give tert-butyl 4-[8-fluoro-6-(7-fluoro-2-methyl-indazol-5-yl)imidazo[1,2-a]pyridin-2-yl]-2-azabicyclo[2.1.1]hexane-2-carboxylate (50 mg, 56% yield) as a yellow solid. MS: m/z 466.6 [M+H]⁺

Step b: tert-butyl 4-[8-fluoro-6-(7-fluoro-2-methyl-indazol-5-yl)imidazo[1,2-a]pyridin-2-yl]-2-azabicyclo[2.1.1]hexane-2-carboxylate (50 mg, 107 μmol) was dissolved in 4 M HCl in EtOAc (20 mL) and the mixture was stirred at 25° C. for 1 h. The mixture was concentrated in vacuo and purified by preparative HPLC (Boston Prime C18 150*30 mm*5 um; water NH₃H₂O+NH₄HCO₃)-ACN) to give 2-(2-azabicyclo[2.1.1]hexan-4-yl)-8-fluoro-6-(7-fluoro-2-methyl-indazol-5-yl)imidazo[1,2-a]pyridine (4.1 mg, 10% yield). MS: m/z 366.2 [M+H]⁺

Step c: To a solution of 2-(2-azabicyclo[2.1.1]hexan-4-yl)-8-fluoro-6-(7-fluoro-2-methyl-indazol-5-yl)imidazo[1,2-a]pyridine (30 mg, 82.1 μmol) in 1,2-dichloroethane (0.4 mL) and EtOH (2 mL) was added paraformaldehyde (98.5 mg, 82.1 μmol, 112 μL) and TEA (164 μmol, 23 μL) and the mixture was stirred at 25° C. for 10 min. Then sodium triacetoxyborohydride (35 mg, 164 μmol) was added and the mixture was stirred at 25° C. for 0.5 h. The mixture was filtered and the filtrate was concentrated in vacuo to give the residue, which was purified by preparative HPLC (Boston Prime C18 150*30 mm*5 um; water NH₃H₂O+NH₄HCO₃)-ACN) to give 7-fluoro-5-[8-fluoro-2-(2-methyl-2-azabicyclo[2.1.1]hexan-4-yl)imidazo[1,2-a]pyridin-6-yl]-2-methyl-indazole (6.2 mg, 20% yield) as a white solid. MS: m/z 380.3 [M+H]⁺; ¹H NMR (500 MHz, METHANOL-d4) δ ppm=8.59 (d, J=1.37 Hz, 1H), 8.38 (d, J=2.59 Hz, 1H), 7.89 (d, J=2.90 Hz, 1H), 7.82 (d, J=1.22 Hz, 1H), 7.52 (dd, J=12.05, 1.37 Hz, 1H), 7.37 (dd, J=12.66, 1.37 Hz, 1H), 4.26 (s, 3H), 3.66 (s, 1H), 3.27-3.14 (m, 2H), 2.69 (s, 3H), 2.28 (s, 2H), 2.08-1.99 (m, 2H).

Using the procedure described for Example 2 above, additional compounds described herein were prepared by substituting the appropriate bromo ketone and aminopyridine starting materials in step a, suitable reagents and reaction conditions, obtaining compounds such as those selected from:

| Cpd ID | Data |
|---|---|
| 80 | Starting materials: tert-butyl 4-(2-bromoacetyl)-2-azabicyclo[2.1.1]hexane-2- |
| | carboxylate and intermediate 3 |
| | MS: m/z 377.2 [M + H]⁺; RT: 1.60 min (Method 7) |
| 76 | Starting materials: 2-bromo-1-(2-methyl-2-azabicyclo[3.1.1]heptan-5- |
| | yl)ethan-1-one and intermediate 4 |
| | MS: m/z 394.3 [M + H]⁺; RT: 1.47 min (Method 7) |
| 109 | Starting materials: tert-butyl (3-(2-bromoacetyl)bicyclo[1.1.1 ]pentan-1- |
| | yl)carbamate and intermediate 3 |
| | MS: m/z 391.3 [M + H]⁺; RT: 1.63 min (Method 7) |
| 209 | Starting materials: tert-butyl 7-(2-bromoacetyl)-4-azaspiro[2.5]octane-4- |
| | carboxylate |
| | MS: m/z 405.4 [M + H]⁺; RT: 0.87 min (Method 3) |
| | ¹H NMR (600 MHz, DMSO-d₆) δ ppm 0.89 (dt, J = 9.73, 6.01 Hz, 1 H) 0.99 |
| | (dt, J = 9.82, 5.96 Hz, 1 H) 1.04-1.10 (m, 1 H) 1.16-1.22 (m, 1 H) 1.57 (dd, |
| | J = 14.50, 3.81 Hz, 1 H) 2.17-2.22 (m, 2 H) 2.44 (s, 3 H) 2.63 (s, 3 H) 2.99 (d, |
| | J = 4.96 Hz, 3 H) 3.18-3.28 (m, 2 H) 3.42-3.47 (m, 1 H) 3.49-3.55 (m, 1 H) |
| | 7.74 (s, 1 H) 7.78 (dd, J = 12.21, 1.14 Hz, 1 H) 8.02 (d, J = 2.67 Hz, 1 H) 8.12 |
| | (s, 1 H) 9.25 (d, J = 1.14 Hz, 1 H) 9.53 (br s, 1 H) |

### Example 3—Compound 79

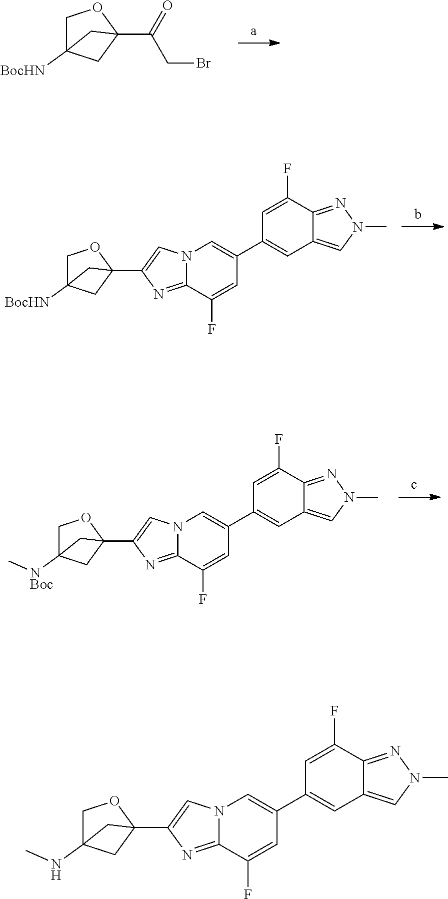

Step a: To a solution of tert-butyl N-[1-(2-bromoacetyl)-2-oxabicyclo[2.1.1]hexan-4-yl]carbamate (92.3 mg, 288.2 μmol) in t-BuOH (5 mL) was added 3-fluoro-5-(7-fluoro-2-methyl-indazol-5-yl)pyridin-2-amine (50 mg, 192.1 μmol) and NaHCO₃ (48.4 mg, 576.39 μmol) and the mixture was stirred at 70° C. for 16 h. The crude material was purified by flash silica gel chromatography (Combi-Flash (PE/EA=3/1 to 1/5). MS: m/z 482.1 [M+H]⁺

Step b: To a solution of tert-butyl N-[1-[8-fluoro-6-(7-fluoro-2-methyl-indazol-5-yl)imidazo[1,2-a]pyridin-2-yl]-2-oxabicyclo[2.1.1]hexan-4-yl]carbamate (100 mg, 207.6 μmol) in THF (5 mL) was added NaH (16.6 mg, 415.3 μmol, 60% purity) at 0° C. and stirred for 0.5 h. Then iodomethane (415.3 μmol, 26 μL) was added and the mixture was stirred at 20° C. for 2 h. The mixture was concentrated and then water (80 mL) was added. The mixture was extracted with EtOAc (50 mL×3). The combined organic layers were washed with brine (50 mL), dried over Na₂SO₄, filtered and concentrated in vacuo to give tert-butyl-N-[1-[8-fluoro-6-(7-fluoro-2-methyl-indazol-5-yl)imidazo[1,2-a]pyridin-2-yl]-2-oxabicyclo[2.1.1]hexan-4-yl]-N-methyl-carbamate (30 mg) which was used crude in the next reaction. MS: m/z 496.1 [M+H]⁺.

Step c: A solution of tert-butyl N-[1-[8-fluoro-6-(7-fluoro-2-methyl-indazol-5-yl)imidazo[1,2-a]pyridin-2-yl]-2-oxabicyclo[2.1.1]hexan-4-yl]-N-methyl-carbamate (30 mg, 60.54 μmol) in TFA (1 mL) and DCM (3 mL) was stirred at rt for 3 h. The mixture was concentrated in vacuo and purified by preparative HPLC (Boston Prime C18 150*30 mm*5 um; water NH₃H₂O+NH₄HCO₃)-ACN) to give 1-[8-fluoro-6-(7-fluoro-2-methyl-indazol-5-yl)imidazo[1,2-a]pyridin-2-yl]-N-methyl-2-oxabicyclo[2.1.1]hexan-4-amine (2.7 mg, 11%). MS: m/z 396.3 [M+H]⁺; ¹H NMR (400 MHz, METHANOL-d4) δ ppm=8.62 (d, J=1.31 Hz, 1H), 8.37 (d, J=2.62 Hz, 1H), 7.97 (d, J=2.98 Hz, 1H), 7.82 (d, J=1.07 Hz, 1H), 7.52 (dd, J=12.10, 1.25 Hz, 1H), 7.37 (dd, J=12.64, 1.19 Hz, 1H), 4.26 (s, 3H), 3.80 (s, 2H), 2.52 (s, 3H), 2.41-2.33 (m, 2H), 2.06-1.95 (m, 2H).

Using the procedure described for Example 3 above, additional compounds described herein were prepared by substituting the appropriate bromo ketone and aminopyridine starting materials in step a, suitable reagents and reaction conditions, obtaining compounds such as those selected from:

| Cpd ID | Data |
|---|---|
| 77 | Starting material: tert-butyl (4-(2-bromoacetyl)bicyclo[2.1.1 |
| | ]hexan-1-yl)carbamate and intermediate 4 |
| | MS: m/z 394.3 [M + H]⁺; RT 1.45 min (Method 7) |
| 100 | Starting material: tert-butyl (3-(2-bromoacetyl)bicyclo[1.1.1 |
| | ]pentan-1-yl)carbamate and intermediate 3 |
| | MS: m/z 377.1 [M + H]⁺; RT 1.214 min (Method 7) |

### Example 4—Compound 161 and/or 185

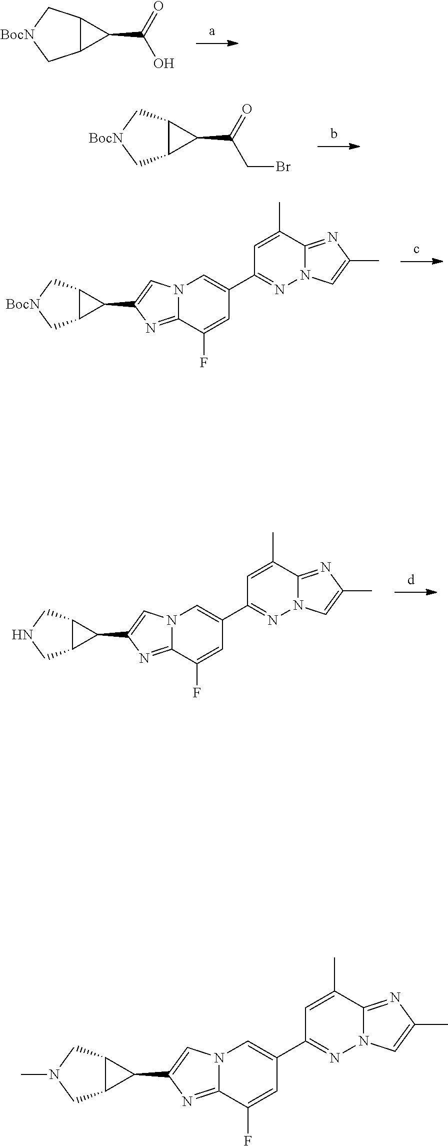

Step a: To a solution of (1S,5R)-3-tert-butoxycarbonyl-3-azabicyclo[3.1.0]hexane-6-carboxylic acid (600 mg, 2.64 mmol) in DCM (4 mL) was added (COCl)₂ (402.14 mg, 3.17 mmol, 268.10 μL) and two drop of DMF at 0° C. The reaction was stirred at the same temperature for 0.5 h and then concentrated in vacuo. The residue was again dissolved in THF (4 mL), DCM (4 mL) and diazomethyl(trimethyl)silane (2 M, 1.32 mL) and the mixture was stirred at 0° C. for 1 h, before HBr (1.60 g, 7.92 mmol, 40% purity) was added at 0° C. The reaction then was stirred at 0° C. for 1 h. The solution was concentrated in vacuo to give tert-butyl (1S,5R)-6-(2-bromoacetyl)-3-azabicyclo[3.1.0]hexane-3-carboxylate (600 mg, crude) as a yellow oil and used for nest step directly.

Step b: To a solution of tert-butyl (1S,5R)-6-(2-bromoacetyl)-3-azabicyclo[3.1.0]hexane-3-carboxylate (300 mg, 986.26 μmol) and 5-(2,8-dimethylimidazo[1,2-b]pyridazin-6-yl)-3-fluoro-pyridin-2-amine (100 mg, 388.70 μmol) in t-BuOH (4 mL) was added NaHCO₃ (165.71 mg, 1.97 mmol, 76.72 μL). The reaction was stirred at 80° C. for 16 h. The mixture was filtered and concentrated and the residue was purified by prep-HPLC. tert-Butyl (1S,5R)-6-[6-(2,8-dimethylimidazo[1,2-b]pyridazin-6-yl)-8-fluoro-imidazo[1,2-a]pyridin-2-yl]-3-azabicyclo[3.1.0]hexane-3-carboxylate (60 mg, 125.55 μmol, 12.7% yield, 96.8% purity) was obtained as a yellow oil. MS: m/z 463.3 [M+H]⁺.

Step c: To a solution of tert-butyl (1S,5R)-6-[6-(2,8-dimethylimidazo[1,2-b]pyridazin-6-yl)-8-fluoro-imidazo[1,2-a]pyridin-2-yl]-3-azabicyclo[3.1.0]hexane-3-carboxylate (20 mg, 43.24 μmol) in EtOAc was added HCl in EtOAc (0.5 mL). The mixture was stirred at 25° C. for 1 h. The mixture was filtered and concentrated. The residue was purified by prep-HPLC. 6-[2-[(1S,5R)-3-azabicyclo[3.1.0]hexan-6-yl]-8-fluoro-imidazo[1,2-a]pyridin-6-yl]-2,8-dimethyl-imidazo[1,2-b]pyridazine (10 mg, 24.83 μmol, 57.4% yield) was obtained as white solid. MS: m/z 363.2 [M+H]⁺; RT: 1.948 min (Method 8); ¹H NMR: (400 MHz, METHANOL-d₄) δ ppm=8.99 (s, 1H), 8.47 (s, 0.5H), 7.93 (s, 1H), 7.90 (s, 1H), 7.80-7.70 (m, 1H), 7.58 (s, 1H), 3.60-3.50 (m, 4H), 2.67 (s, 3H), 2.49 (s, 3H), 2.40-2.30 (m, 2H), 2.15-2.10 (m, 1H).

Step d: To a solution of 6-[2-[(1S,5R)-3-azabicyclo[3.1.0]hexan-6-yl]-8-fluoro-imidazo[1,2-a]pyridin-6-yl]-2,8-dimethyl-imidazo[1,2-b]pyridazine (30 mg, 82.78 μmol) in EtOH (3 mL) was added paraformaldehyde (99.30 mg, 82.78 umol, 112.84 μL), TEA (25.13 mg, 248.34 umol, 34.61 μL) and DCE (16.38 mg, 165.56 μmol, 13.11 μL). The mixture was stirred at 25° C. for 20 min. Then sodium triacetoxyborohydride (35.09 mg, 165.56 μmol) was added and the mixture was stirred at 25° C. for 1 h. The mixture was filtered and the filtrate was concentrated in vacuo. The residue was purified by prep-HPLC (Column: Boston Green ODS 150*30 mm*5 um; Condition: water (FA)-ACN, Begin B 3, End B 33; Gradient Time (min): 12; 100% B Hold Time (min): 2; Flow Rate (ml/min): 25) to give 6-[8-fluoro-2-[(1S,5R)-3-methyl-3-azabicyclo[3.1.0]hexan-6-yl]imidazo[1,2-a]pyridin-6-yl]-2,8-dimethyl-imidazo[1,2-b]pyridazine (4.7 mg, 12.5 μmol, 15.1% yield) as a yellow solid. MS: m/z 377.3 [M+H]⁺; RT: 0.628 min (Method 10); ¹H NMR: (400 MHz, METHANOL-d₄) δ ppm=8.95 (d, J=0.8, 1H), 8.51 (s, 1H), 7.91 (s, 1H), 7.86 (s, 1H), 7.80-7.70 (m, 1H), 7.56 (s, 1H), 3.55-3.50 (m, 3H), 3.20-3.10 (m, 2H), 2.71 (s, 3H), 2.66 (s, 3H), 2.48 (s, 3H), 2.40-2.30 (m, 1H), 2.20-2.10 (m, 2H).

Using the procedure described for Example 4 above, additional compounds described herein were prepared by substituting the appropriate carboxylic acid starting material in step a, and amino pyridine intermediate in step b, suitable reagents, and reaction conditions, obtaining compounds such as those selected from:

| Cpd ID | Data |
|---|---|
| 70 | Starting materials: 4-(tert-butoxycarbonyl)morpholine-2-carboxylic acid |
| | and intermediate 4 |
| | MS: m/z 370.2 [M + H]⁺; RT: 1.48 min (Method 7); stereochemistry |
| | arbitrarily assigned |
| 71 | Starting materials: 4-(tert-butoxycarbonyl)morpholine-2-carboxylic acid |
| | and intermediate 4 |
| | MS: m/z 370.2 [M + H]⁺; RT: 1.47 min (Method 7); stereochemistry |
| | arbitrarily assigned |
| 72 | Starting materials: 4-((tert-butoxycarbonyl)amino)bicyclo[2.1.1]hexane-1- |
| | carboxylic acid and intermediate 4 |
| | MS: m/z 380.2 [M + H]⁺; RT: 1.41 min (Method 7) |
| 75 | Starting materials: 2-(tert-butoxycarbonyl)-2-azabicyclo[3.1.1]heptane-5- |
| | carboxylic acid and intermediate 4 |
| | MS: m/z 380.2 [M + H]⁺; RT: 1.42 min (Method 7) |
| 78 | Starting materials: 2-(tert-butoxycarbonyl)-2-azabicyclo[3.1.1]heptane-5- |
| | carboxylic acid and intermediate 4 |
| | MS: m/z 405.2 [M + H]⁺; RT: 1.42 min (Method 7) |
| | 1H NMR: (400 MHz, METHANOL-d4) δ ppm = 9.60-9.49 (m, 1H), 8.49- |
| | 8.35 (m, 4H), 3.91 (d, J = 7.2 Hz, 1H), 3.25 (s, 2H), 3.15 (s, 2H), 2.87- |
| | 2.81 (m, 3H), 2.71-2.67 (m, 3H), 2.54 (t, J = 9.0 Hz, 2H), 2.30 (t, J = 9.0 |
| | Hz, 2H), 2.07 (s, 2H), 1.94 (s, 2H). |
| 107 | Starting materials: 2-(tert-butoxycarbonyl)-2-azaspiro[3.3]heptane-6- |
| | carboxylic acid and intermediate 3 |
| | MS: m/z 377.0 [M + H]⁺; RT: 1.07 min (method 7) |
| 108 | Starting materials: 4-((tert-butoxycarbonyl)amino)bicyclo[2.2.2]octane-1- |
| | carboxylic acid and intermediate 3 |
| | MS: m/z 405.3 [M + H]⁺; RT: 0.85 min (method 7) |
| 110 | Starting materials: 4-(tert-butoxycarbonyl)-4-azaspiro[2.5]octane-7- |
| | carboxylic acid and intermediate 3 |
| | MS: m/z 391.1 [M + H]⁺; RT: 1.30 min (method 7) |
| 114 | Starting materials: 8-(tert-butoxycarbonyl)-8-azaspiro[4.5]decane-2- |
| | carboxylic acid and intermediate 3 |
| | MS: m/z 419.2 [M + H]⁺; RT: 0.65 min (method 7) |
| | stereochemistry arbitrarily assigned |
| 115 | Starting materials: 2-(tert-butoxycarbonyl)-2-azabicyclo[2.2.1]heptane-4- |
| | carboxylic acid and intermediate 3 |
| | MS: m/z 377.2 [M + H]⁺; RT: 1.82 min (method 7) |
| | stereochemistry arbitrarily assigned |
| 116 | Starting materials: 2-(tert-butoxycarbonyl)-2-azabicyclo[2.2.1]heptane-4- |
| | carboxylic acid and intermediate 3 |
| | MS: m/z 377.2 [M + H]⁺; RT: 1.71 min (method 7) |
| | stereochemistry arbitrarily assigned |
| 117 | Starting materials: 1-(tert-butoxycarbonyl)piperidine-3-carboxylic acid and |
| | intermediate 3 |
| | MS: m/z 365.1 [M + H]⁺; RT: 1.27 min (method 7) |
| | stereochemistry arbitrarily assigned |
| 118 | Starting materials: 1-(tert-butoxycarbonyl)piperidine-3-carboxylic acid and |
| | intermediate 3 |
| | MS: m/z 365.1 [M + H]⁺; RT: 1.22 min (method 7) |
| | stereochemistry arbitrarily assigned |
| 119 | Starting materials: 8-(tert-butoxycarbonyl)-8-azaspiro[4.5]decane-2- |
| | carboxylic acid and intermediate 3 |
| | MS: m/z 419.2 [M + H]⁺; RT: 0.66 min (method 7) |
| | stereochemistry arbitrarily assigned |
| 91 | Starting materials: 3-((tert-butoxycarbonyl)amino)bicyclo[1.1.1]pentane- |
| | 1-carboxylic acid and intermediate 3 |
| | MS: m/z 363.1 [M + H]⁺; RT: 1.433 min (method 7) |
| 158 | Starting material: 1-(tert-butoxycarbonyl)-4-fluoropiperidine-4-carboxylic |
| | acid and intermediate 3 |
| | MS: m/z 383.1 [M + H]⁺; RT: 1.646 min (Method 8) |
| 159 | Starting material: 2-(tert-butoxycarbonyl)-2-azabicyclo[2.1.1]hexane-4- |
| | carboxylic acid and intermediate 3 |
| | MS: m/z 363.1 [M + H]⁺; RT: 0.66 min (Method 8) |
| 160 | Starting material: 1-(tert-butoxycarbonyl)pyrrolidine-3-carboxylic acid and |
| | intermediate 3 |
| | MS: m/z 351.2 [M + H]⁺; RT: 1.523 min (Method 10) |
| | ¹H NMR: (500 MHz, METHANOL-d₄) δ ppm = 2.40-2.43 (m, 1H), 2.70- |
| | 2.80 (m, 4H), 2.85 (s, 3H), 3.54-3.64 (m, 3H), 3.91-3.93 (m, 1H), 4.02- |
| | 4.04 (m, 1H), 8.40-8.41 (m, 2H), 8.54 (s, 1H), 8.57 (s, 1H), 9.61 (s, 1H). |
| 162 | Starting material: (1R,3S)-3-((tert- |
| | butoxycarbonyl)(methyl)amino)cyclohexane-1-carboxylic acid and |
| | intermediate 4 |
| | MS: m/z 396.1 [M + H]⁺; RT: 2.252 min (Method 8) |
| 163 | Starting material: (1S,3R)-3-((tert- |
| | butoxycarbonyl)(methyl)amino)cyclohexane-1-carboxylic acid and |
| | intermediate 4 |
| | MS: m/z 396.1 [M + H]⁺; RT: 2.252 min (Method 8) |
| 168 | Starting material: (1s,4s)-4-(dimethylamino)cyclohexane-1-carboxylic acid |
| | and intermediate 3 |
| | MS: m/z 407.1 [M + H]⁺; RT: 1.988 min (Method 8) |
| | ¹H NMR: (400 MHz, METHANOL-d₄) δ ppm = 1.76-1.88 (m, 4H), 2.25- |
| | 2.36 (m, 2H), 2.36-2.47 (m, 2H), 2.68 (s, 3H), 2.84 (s, 3H), 2.92 (s, 6H), |
| | 3.02-3.15 (m, 1H), 3.36-3.49 (m, 1H), 8.32 (d, J = 1.6 Hz, 1H), 8.39 (d, |
| | J = 11.6 Hz, 2H), 8.51 (d, J = 10.8 Hz, 1H), 9.55 (s, 1H). |
| 170 | Starting material: rel-(1R,4R)-4-((tert- |
| | butoxycarbonyl)(methyl)amino)cyclohexane-1-carboxylic acid and |
| | intermediate 4 |
| | MS: m/z 396.1 [M + H]⁺; RT: 1.722 min (Method 8) |
| | ¹H NMR: (400 MHz, METHANOL-d₄) δ ppm = 1.28-1.38 (m, 2H), 1.56- |
| | 1.67 (m, 2H), 2.13-2.22 (m, 4H), 2.46 (s, 3H), 2.53-2.61 (m, 1H), 2.72- |
| | 2.80 (m, 1H), 4.25 (s, 3H), 7.34 (dd, J = 12.8, 1.2 Hz, 1H), 7.44 (dd, J = |
| | 12.0, 1.2 Hz, 1H), 7.75 (d, J = 2.8 Hz, 1H), 7.78 (d, J = 1.2 Hz, 1H), 8.36 |
| | (d, J = 2.8 Hz, 1H), 8.53 (d, J = 1.2 Hz, 1H). |
| 176 | Starting material: (2S,4S,7R)-6-(tert-butoxycarbonyl)-7-methyl-6- |
| | azaspiro[3.4]octane-2-carboxylic acid and intermediate 3 |
| | MS: m/z 405.1 [M + H]⁺; RT: 1.267 min (Method 10) |
| | ¹H NMR: (400 MHz, METHANOL-d₄) δ ppm = 1.47 (d, J = 6.8 Hz, 3H), |
| | 1.85-1.91 (m, 1H), 2.49 (s, 6H), 2.56-2.65 (m, 2H), 2.68 (s, 3H), 3.34- |
| | 3.42 (m, 2H), 3.69-3.79 (m, 2H), 7.59 (s, 1H), 7.81-7.85 (m, 1H), 7.87 |
| | (d, J = 3.2 Hz, 1H), 7.94 (s, 1H), 8.44 (s, 1H), 9.01 (d, J = 1.6 Hz, 1H). |
| 177 | Starting material: (2S,4R,7S)-6-(tert-butoxycarbonyl)-7-methyl-6- |
| | azaspiro[3.4]octane-2-carboxylic acid and intermediate 3 |
| | MS: m/z 405.1 [M + H]⁺; RT: 1. 254 min (Method 10) |
| | ¹H NMR: (400 MHz, METHANOL-d₄) δ: ppm = 1.42 (d, J = 6.8 Hz, 3H), |
| | 1.77-1.83 (m, 1H), 2.34-2.39 (m, 1H), 2.46-2.50 (m, 4H), 2.53-2.61 |
| | (m, 3H), 2.67 (s, 3H), 3.46-3.57 (m, 2H), 3.67-3.76 (m, 2H), 7.57 (s, 1H), |
| | 7.79-7.82 (m, 1H), 7.89 (d, J = 2.8 Hz, 1H), 7.93 (s, 1H), 8.52 (s, 1H), 8.99 |
| | (d, J = 1.6 Hz, 1H). |
| 178 | Starting material: (2R,4R,7R)-6-(tert-butoxycarbonyl)-7-methyl-6- |
| | azaspiro[3.4]octane-2-carboxylic acid and intermediate 3 |
| | MS: m/z 405.1 [M + H]⁺; RT: 1.253 min (Method 10) |
| 179 | Starting material: (S)-4-(tert-butoxycarbonyl)-4-azaspiro[2.5]octane-7- |
| | carboxylic acid and intermediate 3 |
| | MS: m/z 391.1 [M + H]⁺; RT: 1.603 min (Method 10) |
| 180 | Starting material: (R)-4-(tert-butoxycarbonyl)-4-azaspiro[2.5]octane-7- |
| | carboxylic acid and intermediate 3 |
| | MS: m/z 391.1 [M + H]⁺; RT: 1.602 min (Method 10) |
| 181 | Starting material: (1R,3R)-3-((dimethylamino)methyl)cyclobutane-1- |
| | carboxylic acid and intermediate 3 |
| | MS: m/z 393.3 [M + H]⁺; RT: 0.887 min (Method 10) |
| | ¹H NMR: (500 MHz, METHANOL-d₄) δ ppm = 2.33-2.42 (m, 2H), 2.47 |
| | (s, 3H), 2.52-2.58 (m, 2H), 2.65 (s, 3H), 2.84 (s, 6H), 2.94-3.04 (m, 1H), |
| | 3.29-3.31 (m, 2H), 3.71-3.80 (m, 1H), 7.54 (s, 1H), 7.76 (dd, J = 12.0, |
| | 1.0 Hz, 1H), 7.89 (s, 1H), 7.92 (d, J = 2.5 Hz, 1H), 8.97 (s, 1 H). |
| 182 | Starting material: (1S,3S)-3-((dimethylamino)methyl)cyclobutane-1- |
| | carboxylic acid and intermediate 3 |
| | MS: m/z 393.3 [M + H]⁺; RT: 0.887 min (Method 10) |
| | ¹H NMR: (500 MHz, METHANOL-d₄) δ ppm = 2.05-2.14 (m, 2H), 2.48 |
| | (s, 3H), 2.50 (s, 6H), 2.57-2.63 (m, 2H), 2.64-2.70 (m, 4H), 2.77-2.84 |
| | (m, 2H), 3.56-3.67 (m, 1H), 7.56 (s, 1H), 7.77 (d, J = 11.0 Hz, 1H), 7.84 |
| | (d, J = 3.0 Hz, 1H), 7.91 (s, 1H), 8.97 (s, 1H). |
| 183 | Starting material: 3-(tert-butoxycarbonyl)-3-azabicyclo[3.2.1]octane-8- |
| | carboxylic acid and intermediate 3 |
| | MS: m/z 391.2 [M + H]⁺; RT: 0.690 min (Method 3) |
| 184 | Starting material: 3-(1-(tert-butoxycarbonyl)azetidin-3- |
| | yl)bicyclo[1.1.1]pentane-1-carboxylic acid and intermediate 3 |
| | MS: m/z 403.3 [M + H]⁺; RT: 0.69 min (Method 3) |
| 186 | Starting material: 1-methylpyrrolidine-3-carboxylic acid and intermediate |
| | 3 |
| | MS: m/z 365.1 [M + H]⁺; RT: 1.568 min (Method 10) |
| 189 | Starting material: (R)-1-methylpyrrolidine-3-carboxylic acid and |
| | intermediate 3 |
| | MS: m/z 351.1 [M + H]⁺; RT: 1.559 min (Method 10) |
| 190 | Starting material: (S)-1-methylpyrrolidine-3-carboxylic acid and |
| | intermediate 3 |
| | MS: m/z 351.1 [M + H]⁺; RT: 1.550 min (Method 10) |
| 191 | Starting material: (2R,4R)-6-(tert-butoxycarbonyl)-6-azaspiro[3.4]octane- |
| | 2-carboxylic acid and intermediate 3 |
| | MS: m/z 391.0 [M + H]⁺; RT: 1.960 min (Method 8) |
| | ¹H NMR: (500 MHz, METHANOL-d₄) δ ppm = 2.33 (t, J = 7.5 Hz, 2H), |
| | 2.65-2.60 (m, 4H), 2.69 (s, 3H), 2.86 (s, 3H), 3.35 (s, 2H), 3.41 (t, J = 7.0 |
| | Hz, 2H), 3.92-4.0 (m, 1H), 8.41-8.48 (m, 3H), 8.59 (d, J = 10.0 Hz, 1H), |
| | 9.63 (s, 1H). |
| 193 | Starting material: (2S,4S)-6-(tert-butoxycarbonyl)-6-azaspiro[3.4]octane- |
| | 2-carboxylic acid and intermediate 3 |
| | MS: m/z 391.1 [M + H]⁺; RT: 1.563 min (Method 10) |
| | ¹H NMR: (500 MHz, METHANOL-d₄) δ ppm = 2.17 (t, J = 7.5 Hz, 2H), |
| | 2.53-2.61 (m, 2H), 2.68 (s, 3H), 2.71-2.76 (m, 2 H), 2.84 (s, 3H), 3.34 (t, |
| | J = 7.0 Hz, 2H), 3.50 (s, 2H), 3.87-3.95 (m, 1 H), 8.38 (d, J = 9.0 Hz, 3H), |
| | 8.47 (d, J = 10.0 Hz, 1H), 9.55 (s, 1H). |
| 202 | Starting material: (2R,4R)-6-(tert-butoxycarbonyl)-6-azaspiro[3.4]octane- |
| | 2-carboxylic acid and intermediate 3 |
| | MS: m/z 405.2 [M + H]⁺; RT: 1.253 min (Method 10) |
| 203 | Starting material: (2S,4S)-6-(tert-butoxycarbonyl)-6-azaspiro[3.4]octane- |
| | 2-carboxylic acid and intermediate 3 |
| | MS: m/z 405.2 [M + H]⁺; RT: 1.253 min (Method 10) |
| 206 | Starting material: 8-(tert-butoxycarbonyl)-8-azabicyclo[3.2.1]octane-3- |
| | carboxylic acid and intermediate 3 |
| | MS: m/z 391.3 [M + H]⁺; RT: 0.84 min (Method 3) |
| 229 | Starting material: 8-(tert-butoxycarbonyl)-8-azaspiro[4.5]decane-2- |
| | carboxylic acid and intermediate 3 |
| | MS: m/z 433.2 [M + H]⁺; RT: 1.583 min (Method 10) |
| 231 | Starting material: (1R,6S,7R)-3-(tert-butoxycarbonyl)-3- |
| | azabicyclo[4.1.0]heptane-7-carboxylic acid and intermediate 3 |
| | MS: m/z 377.3 [M + H]⁺; RT: 1.214 min (Method 10) |
| | ¹H NMR: (400 MHz, METHANOL-d₄) δ ppm = 1.75-1.86 (m, 2H), 2.16- |
| | 2.23 (m, 2H), 2.35-2.44 (m, 1H), 2.49 (s, 3H), 2.65-2.68 (m, 6H), 2.75- |
| | 2.80 (m, 1H), 2.93-2.96 (m, 1H), 3.13-3.18 (m, 1H), 3.53-3.57 (m, 1H), |
| | 7.58 (s, 1H), 7.79 (d, J = 12.4 Hz, 1H), 7.86 (d, J = 2.8 Hz, 1H), 7.93 (s, |
| | 1H), 8.97 (s, 1H). |
| 234 | Starting material: tert-butyl 4-(2-bromoacetyl)-2,2-dimethyl-piperidine-1- |
| | carboxylate |
| | MS: m/z 379.2 [M + H]⁺; RT: 0.38 min (Method 4) |
| | 1H NMR (400 MHz, METHANOL-d4) δ ppm 1.27-1.31 (m, 3 H) 1.33- |
| | 1.39 (m, 3 H) 1.57-1.73 (m, 3 H) 2.01-2.08 (m, 1 H) 2.11-2.19 (m, 1 H) |
| | 2.46-2.52 (m, 3 H) 3.06-3.15 (m, 2 H) 7.72-7.78 (m, 1 H) 7.81-7.89 |
| | (m, 2 H) 7.97-8.03 (m, 2 H) 9.02-9.05 (m, 1 H) |
| 235 | Starting material: tert-butyl 4-(2-bromoacetyl)-2,2-dimethyl-piperidine-1- |
| | carboxylate |
| | MS: m/z 379.2 [M + H]⁺; RT: 0.38 min (Method 4) |
| | 1H NMR (400 MHz, METHANOL-d4) δ ppm 1.28-1.33 (m, 3 H) 1.35- |
| | 1.39 (m, 3 H) 1.60-1.74 (m, 3 H) 2.02-2.08 (m, 1 H) 2.13-2.18 (m, 1 H) |
| | 2.48-2.52 (m, 3 H) 3.08-3.15 (m, 2 H) 7.73-7.77 (m, 1 H) 7.82-7.89 |
| | (m, 2 H) 7.98-8.02 (m, 2 H) 9.03-9.05 (m, 1 H) |
| 232 | Starting material: 2-(tert-butoxycarbonyl)-2-azaspiro[4.4]nonane-7- |
| | carboxylic acid and intermediate 3 |
| | MS: m/z 419.3 [M + H]⁺; RT: 1.60 min (Method 10) |
| 236 | Starting material: tert-butyl 4-(3-bromo-2-oxo-propyl)piperidine-1- |
| | carboxylate and intermediate 14 |
| | MS: m/z 382.3 [M + H]⁺; RT: 0.34 min (Method 4) |
| 237 | Starting material: 8-fluoro-6-(8-fluoro-2-methyl-imidazo[1,2-a]pyridin-6- |
| | yl)-2-(4-piperidylmethyl)imidazo[1,2-a]pyridine and paraformaldehyde |
| | MS: m/z 393.4 [M + H]⁺; RT: 0.37 min (Method 4) |
| 238 | Starting material: tert-butyl 4-(3-bromo-2-oxo-propyl)piperidine-1- |
| | carboxylate and intermediate 3 |
| | MS: m/z 379.4 [M + H]⁺; RT: 0.37 min (Method 4) |
| 239 | Starting material: tert-butyl 4-(2-bromoacetyl)-4-methyl-piperidine-1- |
| | carboxylate and intermediate 3 |
| | MS: m/z 379.3 [M + H]⁺; RT: 0.39 min (Method 4) |
| 240 | Starting material: tert-butyl 4-(2-bromoacetyl)-4-methyl-piperidine-1- |
| | carboxylate and intermediate 14 |
| | MS: m/z 382.3 [M + H]⁺; RT: 0.38 min (Method 4) |
| 241 | Starting material: tert-butyl 7-(2-bromoacetyl)-4-azaspiro[2.5]octane-4- |
| | carboxylate and intermediate 14 |
| | MS: m/z 392.4 [M + H]⁺; RT: 0.44 min (Method 4) |
| 242 | Starting material: tert-butyl 4-(2-bromoacetyl)-2,2-dimethyl-piperidine-1- |
| | carboxylate and intermediate 14 |
| | MS: m/z 396.4 [M + H]⁺; RT: 0.38 min (Method 4) |

### Example 5—Compound 1

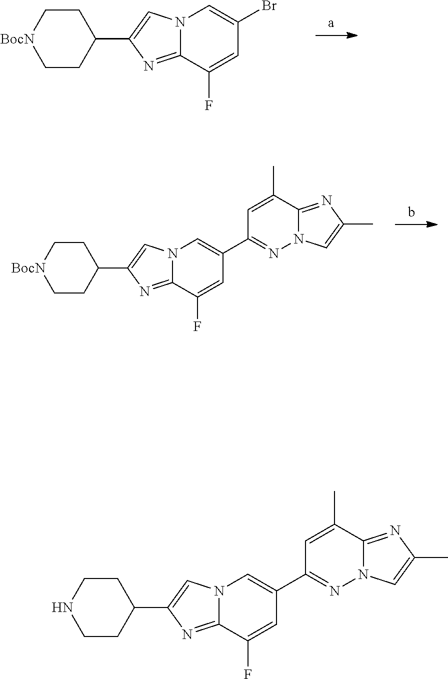 [SMILES: CC(C)(C)OC(=O)N1CCC(c2cn3cc(Br)cc(F)c3n2)CC1 ; Cc1cn2nc(-c3cc(F)c4nc(C5CCN(C(=O)OC(C)(C)C)CC5)cn4c3)cc(C)c2n1 ; Cc1cn2nc(-c3cc(F)c4nc(C5CCNCC5)cn4c3)cc(C)c2n1]

Step a: To a solution of tert-butyl 4-[8-fluoro-6-(4,4,5,5-tetramethyl-1,3,2-dioxaborolan-2-yl)imidazo[1,2-a]pyridin-2-yl]piperidine-1-carboxylate (550 mg, 1.24 mmol), 6-chloro-2,8-dimethyl-imidazo[1,2-b]pyridazine (224.31 mg, 1.24 mmol) and K₂CO₃ (512 mg, 3.71 mmol) in Dioxane (1 mL) and water (0.5 mL) was added Pd(dppf)Cl₂ (90.3 mg, 123.5 μmol) at 25° C. under N₂. The mixture was stirred at 90° C. for 2 hours. The mixture was filtered and concentrated to give crude product. The crude was purified by flash silica gel chromatography (from PE to EtOAc, 0-100%) to yield tert-butyl 4-[6-(2,8-dimethylimidazo[1,2-b]pyridazin-6-yl)-8-fluoro-imidazo[1,2-a]pyridin-2-yl]piperidine-1-carboxylate (270 mg, 47% yield) as a white solid. MS: m/z 465.2 [M+H]⁺

Step b: To a solution of tert-butyl 4-[6-(2,8-dimethylimidazo[1,2-a]pyridin-6-yl)-8-fluoro-imidazo[1,2-a]pyridin-2-yl]piperidine-1-carboxylate (60 mg, 0.129 mmol) in DCM (5 mL) was added TFA (6.53 mmol, 0.5 mL) at 20° C. The mixture was stirred at 20° C. for 20 min. The mixture was concentrated to give the residue, which was purified by prep-HPLC (Column: Waters Xbridge BEH C1 100*25 mm*5 um; Condition: water (0.225% FA)-ACN; Begin B: 0; End B: 20; Gradient Time (min): 12; 100d, B Hold Time (min): 2; FlowRate (ml/min): 25) to give 6-(2,8-dimethylimidazo[1,2-a]pyridin-6-yl)-8-fluoro-2-(4-piperidyl)imidazo[1,2-a]pyridine (43 mg, 91% yield) as an off-white solid. MS: m/z 365.1 [M+H]+; ¹H NMR: (400 MHz, METHANOL-d4) δ ppm=9.28 (s, 1H), 8.19-8.35, (m, 2H), 7.98-8.11 (m, 2H), 3.55 (br d, J=12.8 Hz, 2H), 3.15-3.30 (m, 3H), 2.71-2.87 (m, 3H), 2.65 (s, 3H), 2.29-2.41 (m, 2H), 1.97-2.13 (m, 2H).

Using the procedure described for Example 5 above, additional compounds described herein were prepared by substituting the appropriate boronic acid or ester equivalent starting material in step a, suitable reagents and reaction conditions, obtaining compounds such as those selected from:

| Cpd ID | Data |
|---|---|
| 29 | Starting material: 1-methyl-6-(4,4,5,5-tetramethyl-1,3,2-dioxaborolan-2- |
| | yl)-3a,7a-dihydro-1H-benzo[d][1,2,3]triazole |
| | MS: m/z 351.2 [M + H]⁺; RT: 0.83 min (Method 5) |
| | ¹H NMR (400 MHz, METHANOL-d4) δ ppm 2.02-2.12 (m, 2 H), 2.29- |
| | 2.38 (m, 2 H), 3.17-3.26 (m, 3 H), 3.51-3.58 (m, 2 H), 4.42 (s, 3 H), |
| | 7.63 (dd, J = 11.76, 1.25 Hz, 1 H), 7.77 (d, J = 10.01 Hz, 1 H), 7.93 (d, |
| | J = 3.00 Hz, 1 H), 8.07-8.11 (m, 3 H), 8.73 (d, J = 1.50 Hz, 1 H) |
| 41 | Starting material: 4-ethoxy-2-(4,4,5,5-tetramethyl-1,3,2-dioxaborolan-2- |
| | yl)furo[3,2-c]pyridine |
| | MS: m/z 381.2 [M + H]⁺; RT: 0.92 min (Method 5) |
| | ¹H NMR (500 MHz, DMSO-d₆) δ ppm 1.39 (t, J = 6.97 Hz, 2 H), 1.80- |
| | 1.92 (m, 2 H), 2.15 (br d, J = 12.24 Hz, 2 H), 2.34 (br s, 1 H), 2.62 (br s, 1 |
| | H), 2.75 (br d, J = 5.12 Hz, 1 H), 3.00-3.11 (m, 3 H), 4.49 (q, J = 7.12 Hz, |
| | 2 H), 7.31 (br d, J = 5.69 Hz, 1 H), 7.60 (s, 1 H), 7.81 (br d, J = 12.24 Hz, 1 |
| | H), 7.98 (br d, J = 2.56 Hz, 1 H), 8.03 (d, J = 5.98 Hz, 1 H), 8.25-8.35 (m, |
| | 1 H), 8.53-8.63 (m, 1 H), 8.96 (s, 1 H) |
| 30 | Starting material: 1-ethyl-3-methyl-7-(4,4,5,5-tetramethyl-1,3,2- |
| | dioxaborolan-2-yl)pyrrolo[1,2-a]pyrazine |
| | MS: m/z 378.1 [M + H]⁺; RT: 0.70 min (Method 5) |
| | ¹H NMR (500 MHz, DMSO-d₆) δ ppm 1.41 (t, J = 7.54 Hz, 3 H), 1.82- |
| | 1.94 (m, 2 H), 2.16 (br d, J = 11.96 Hz, 2 H), 2.41 (s, 3 H), 3.03-3.10 (m, |
| | 3 H), 3.12-3.19 (m, 2 H), 3.38 (br s, 2 H), 7.73 (br d, J = 12.24 Hz, 1 H), |
| | 7.90 (d, J = 2.56 Hz, 1 H), 8.36 (br s, 1 H), 8.56-8.68 (m, 2 H), 8.98 (s, 1 |
| | H) |
| 95 | Starting material: 2,8-dimethyl-6-(4,4,5,5-tetramethyl-1,3,2- |
| | dioxaborolan-2-yl)imidazo[1,2-a]pyridine |
| | MS: m/z 364.2 [M + H]⁺; RT: 0.70 min (Method 5) |
| | ¹H NMR (500 MHz, DMSO-d₆) δ ppm 1.82-1.96 (m, 2 H), 2.16 (br d, |
| | J = 12.81 Hz, 2 H), 2.51 (s, 3 H), 2.62 (s, 3 H), 3.02-3.15 (m, 3 H), 3.37 |
| | (br d, J = 11.96 Hz, 2 H), 7.63 (d, J = 12.53 Hz, 1 H), 7.94 (d, J = 2.56 Hz, 1 |
| | H), 8.05 (s, 1 H), 8.09 (br s, 1 H), 8.63 (br dd, J = 8.68, 1.57 Hz, 1 H), 8.90 |
| | (s, 1 H), 9.11 (s, 1 H) |
| 39 | Starting material: 2-(6-chlorobenzofuran-2-yl)-4,4,5,5-tetramethyl-1,3,2- |
| | dioxaborolane |
| | MS: m/z 372.0 [M + H]⁺; RT: 1.12 min (Method 5) |
| | ¹H NMR (500 MHz, DMSO-d₆) δ ppm 1.80-1.91 (m, 2 H), 2.10-2.19 |
| | (m, 2 H), 2.52 (s, 1 H), 2.75 (d, J = 4.93 Hz, 1 H), 2.99-3.12 (m, 3 H), 7.33 |
| | (dd, J = 8.22, 1.64 Hz, 1 H), 7.52 (s, 1 H), 7.67-7.81 (m, 3 H), 8.02 (d, |
| | J = 2.96 Hz, 1 H), 8.55-8.63 (m, 1 H), 8.95 (s, 1 H) |
| 38 | Starting material: 2-methyl-4-(4,4,5,5-tetramethyl-1,3,2-dioxaborolan-2- |
| | yl)benzonitrile |
| | MS: m/z 335.2 [M + H]⁺; RT: 0.82 min (Method 5) |
| | ¹H NMR (500 MHz, DMSO-d₆) δ ppm 1.81-1.94 (m, 2 H), 2.15 (br d, |
| | J = 12.24 Hz, 2 H), 2.54 (s, 3 H), 3.01-3.12 (m, 3 H), 3.36 (br d, J = 12.24 |
| | Hz, 2 H), 7.64 (d, J = 12.24 Hz, 1 H), 7.72 (br d, J = 8.26 Hz, 1 H), 7.83- |
| | 7.91 (m, 3 H), 8.58 (br dd, J = 8.26, 1.99 Hz, 1 H), 8.90 (s, 1 H) |
| 96 | Starting material: 2-methyl-6-(4,4,5,5-tetramethyl-1,3,2-dioxaborolan-2- |
| | yl)imidazo[1,2-a]pyridine |
| | MS: m/z 351.2 [M + H]⁺; RT: 0.46 min (Method 5) |
| | ¹H NMR (500 MHz, DMSO-d₆) δ ppm 1.82-1.94 (m, 2 H), 2.16 (br d, |
| | J = 11.96 Hz, 2 H), 2.52 (s, 3 H), 3.04-3.12 (m, 3 H), 3.25-3.28 (m, 2 H), |
| | 7.62 (d, J = 12.24 Hz, 1 H), 7.92-7.99 (m, 3 H), 8.07 (br d, J = 9.11 Hz, 1 |
| | H), 8.56-8.70 (m, 1 H), 8.88 (s, 1 H), 9.18 (s, 1 H) |
| 43 | Starting material: 2-(4-fluorobenzo[b]thiophen-2-yl)-4,4,5,5-tetramethyl- |
| | 1,3,2-dioxaborolane |
| | MS: m/z 370.0 [M + H]⁺; RT: 1.16 min (Method 5) |
| | ¹H NMR (500 MHz, DMSO-d₆) δ ppm 1.79-1.92 (m, 2 H), 2.12-2.20 |
| | (m, 2 H), 3.00-3.13 (m, 3 H), 3.35 (br d, J = 3.29 Hz, 2 H), 7.20-7.27 (m, |
| | 1 H), 7.41 (td, J = 7.97, 5.10 Hz, 1 H), 7.80-7.89 (m, 2 H), 7.95 (d, J = 2.63 |
| | Hz, 1 H), 8.02 (s, 1 H), 8.54-8.66 (m, 1 H), 8.89 (s, 1 H) |
| 36 | Starting material: 2-(difluoromethyl)-5-(4,4,5,5-tetramethyl-1,3,2- |
| | dioxaborolan-2-yl)-2H-indazole |
| | MS: m/z 386.2 [M + H]⁺; RT: 0.65 min (Method 5) |
| | ¹H NMR (500 MHz, DMSO-d₆) δ ppm 1.81-1.93 (m, 2 H), 2.16 (br d, |
| | J = 12.83 Hz, 2 H), 3.00-3.14 (m, 3 H), 3.36 (br d, J = 12.83 Hz, 2 H), 7.63 |
| | (d, J = 12.17 Hz, 1 H), 7.74 (d, J = 9.21 Hz, 1 H), 7.85 (d, J = 8.88 Hz, 1 H), |
| | 7.90 (d, J = 2.96 Hz, 1 H), 8.03-8.28 (m, 2 H), 8.59 (br dd, J = 9.04, 2.80 |
| | Hz, 1 H), 8.82 (s, 1 H), 8.96 (s, 1 H) |
| 31 | Starting material: 1,3-dimethyl-7-(4,4,5,5-tetramethyl-1,3,2- |
| | dioxaborolan-2-yl)pyrrolo[1,2-a]pyrazine |
| | MS: m/z 364.2 [M + H]⁺; RT: 0.69 min (Method 5) |
| | ¹H NMR (500 MHz, DMSO-d₆) δ ppm 1.81-1.93 (m, 2 H), 2.16 (br d, |
| | J = 13.10 Hz, 2 H), 2.40 (s, 3 H), 2.83 (s, 3 H), 3.03-3.11 (m, 3 H), 3.38 |
| | (br s, 2 H), 7.71 (br d, J = 11.96 Hz, 1 H), 7.90 (d, J = 2.56 Hz, 1 H), 8.36 |
| | (br s, 2 H), 8.57 (br s, 1 H), 8.60-8.68 (m, 1 H), 8.98 (s, 1 H) |
| 40 | Starting material: 3-ethyl-5-(4,4,5,5-tetramethyl-1,3,2-dioxaborolan-2- |
| | yl)-1H-pyrrolo[2,3-b]pyridine |
| | MS: m/z 364.0 [M + H]⁺; RT: 0.92 min (Method 5) |
| | ¹H NMR (500 MHz, DMSO-d₆) δ ppm 1.28 (t, J = 7.54 Hz, 3 H), 1.87 (q, |
| | J = 11.29 Hz, 2 H), 2.17 (br d, J = 12.81 Hz, 2 H), 2.75 (q, J = 7.69 Hz, 2 H), |
| | 3.02-3.14 (m, 3 H), 3.37 (br d, J = 12.24 Hz, 2 H), 7.30 (s, 1 H), 7.71 (br |
| | d, J = 12.24 Hz, 1 H), 7.91 (d, J = 2.28 Hz, 1 H), 8.25 (s, 1 H), 8.52 (d, J = 1.71 |
| | Hz, 1 H), 8.58 (br dd, J = 6.55, 2.85 Hz, 1 H), 8.80 (s, 1 H), 11.46 (br s, 1 |
| | H) |
| 42 | Starting material: 2-methyl-6-(4,4,5,5-tetramethyl-1,3,2-dioxaborolan-2- |
| | yl)imidazo[1,2-a]pyridine-8-carbonitrile |
| | MS: m/z 375.0 [M + H]⁺; RT: 0.79 min (Method 5) |
| | ¹H NMR (500 MHz, DMSO-d₆) δ ppm 1.83-1.94 (m, 2 H), 2.16 (br d, |
| | J = 13.38 Hz, 2 H), 2.41 (s, 3 H), 3.02-3.12 (m, 3 H), 3.36 (br d, J = 11.96 |
| | Hz, 2 H), 7.64 (d, J = 12.53 Hz, 1 H), 7.85-7.93 (m, 2 H), 8.33 (s, 2 H), |
| | 8.86 (s, 1 H), 9.22 (s, 1 H) |
| 35 | Starting material: 3-methyl-6-(4,4,5,5-tetramethyl-1,3,2-dioxaborolan-2- |
| | yl)thieno[3,2-b]pyridine |
| | MS: m/z 367.0 [M + H]⁺; RT: 0.96 min (Method 5) |
| | ¹H NMR (500 MHz, DMSO-d₆) δ ppm 1.83-1.93 (m, 2 H), 2.17 (br d, |
| | J = 13.66 Hz, 2 H), 2.45 (s, 3 H), 3.02-3.12 (m, 3 H), 3.37 (br d, J = 12.24 |
| | Hz, 2 H), 7.71 (d, J = 12.24 Hz, 1 H), 7.83 (s, 1 H), 7.93 (d, J = 2.56 Hz, 1 |
| | H), 8.53-8.66 (m, 1 H), 8.77 (d, J = 1.42 Hz, 1 H), 8.90 (s, 1 H), 9.01 (d, |
| | J = 1.42 Hz, 1 H) |
| 32 | Starting material: 8-chloro-2-methyl-6-(4,4,5,5-tetramethyl-1,3,2- |
| | dioxaborolan-2-yl)imidazo[1,2-a]pyridine |
| | MS: m/z 384.2 [M + H]⁺; RT: 0.62 min (Method 5) |
| | ¹H NMR (400 MHz, METHANOL-d₄) δ ppm 2.01-2.12 (m, 2 H), 2.34 |
| | (br dd, J = 14.26, 2.75 Hz, 2 H), 2.61 (s, 3 H), 3.17-3.27 (m, 3 H), 3.52- |
| | 3.60 (m, 2 H), 7.59 (dd, J = 11.51, 1.50 Hz, 1 H), 7.97 (d, J = 3.00 Hz, 1 |
| | H), 8.05 (d, J = 1.00 Hz, 1 H), 8.30 (d, J = 1.50 Hz, 1 H), 8.77 (d, J = 1.50 |
| | Hz, 1 H), 9.01 (d, J = 1.50 Hz, 1 H) |
| 33 | Starting material: 5-(4,4,5,5-tetramethyl-1,3,2-dioxaborolan-2- |
| | yl)benzo[c]isothiazole |
| | MS: m/z 353.0 [M + H]⁺; RT: 0.77 min (Method 5) |
| | ¹H NMR (400 MHz, METHANOL-d4) δ ppm 2.02-2.13 (m, 2 H), 2.36 |
| | (br dd, J = 14.51, 2.50 Hz, 2 H), 3.19-3.29 (m, 3 H), 3.53-3.59 (m, 2 H), |
| | 7.76 (dd, J = 12.01, 1.50 Hz, 1 H), 7.84-7.89 (m, 1 H), 7.92-7.97 (m, 1 |
| | H), 8.00 (d, J = 2.50 Hz, 1 H), 8.23 (d, J = 1.50 Hz, 1 H), 8.77 (d, J = 1.50 Hz, |
| | 1 H), 9.69 (s, 1 H) |
| 37 | Starting material: 2-methyl-6-(4,4,5,5-tetramethyl-1,3,2-dioxaborolan-2- |
| | yl)-[1,2,4]triazolo[1,5-a]pyridine |
| | MS: m/z 351.2 [M + H]⁺; RT: 0.68 min (Method 5) |
| | ¹H NMR (500 MHz, DMSO-d₆) δ ppm 1.83-1.93 (m, 2 H), 2.16 (br d, |
| | J = 12.81 Hz, 2 H), 2.52 (s, 3 H), 3.02-3.11 (m, 3 H), 3.36 (br d, J = 11.96 |
| | Hz, 2 H), 7.71 (d, J = 12.53 Hz, 1 H), 7.83 (d, J = 9.11 Hz, 1 H), 7.90 (d, |
| | J = 2.56 Hz, 1 H), 7.97 (d, J = 9.39 Hz, 1 H), 8.49-8.65 (m, 1 H), 8.88 (s, 1 |
| | H), 9.26 (s, 1 H) |
| 34 | Starting material: 5-(4,4,5,5-tetramethyl-1,3,2-dioxaborolan-2- |
| | yl)pyrazolo[1,5-a]pyridine |
| | MS: m/z 336.0 [M + H]⁺; RT: 0.74 min (Method 5) |
| | ¹H NMR (500 MHz, DMSO-d₆) δ ppm 1.82-1.92 (m, 2 H), 2.16 (br d, |
| | J = 13.10 Hz, 2 H), 3.02-3.12 (m, 3 H), 3.38 (br s, 2 H), 6.66 (s, 1 H), 7.24 |
| | (br d, J = 7.40 Hz, 1 H), 7.71 (d, J = 12.53 Hz, 1 H), 7.89 (d, J = 2.56 Hz, 1 |
| | H), 8.02-8.11 (m, 2 H), 8.58 (br d, J = 9.68 Hz, 1 H), 8.79 (d, J = 7.40 Hz, |
| | 1 H), 8.94 (s, 1 H) |
| 44 | Starting material: 6-(4,4,5,5-tetramethyl-1,3,2-dioxaborolan-2- |
| | yl)benzo[d]thiazole |
| | MS: m/z 353.2 [M + H]⁺; RT: 0.76 min (Method 5) |
| 93 | Starting material: 8-fluoro-2-methyl-6-(4,4,5,5-tetramethyl-1,3,2- |
| | dioxaborolan-2-yl)imidazo[1,2-a]pyridine |
| | MS: m/z 368.1 [M + H]⁺; RT: 0.44 min (Method 4) |
| | ¹H NMR (400 MHz, METHANOL-d₄) δ ppm 1.99-2.13 (m, 2 H), 2.35 |
| | (br dd, J = 14.01, 1.75 Hz, 2 H), 3.22 (td, J = 12.88, 3.00 Hz, 3 H), 3.51- |
| | 3.60 (m, 2 H), 4.29 (s, 3 H), 7.39 (dd, J = 12.63, 1.38 Hz, 1 H), 7.65 (dd, |
| | J = 12.01, 1.25 Hz, 1 H), 7.86 (d, J = 1.25 Hz, 1 H), 7.95 (d, J = 2.75 Hz, 1 |
| | H), 8.41 (d, J = 2.50 Hz, 1 H), 8.66 (d, J = 1.50 Hz, 1 H) |
| 73 | Starting material: 4-methyl-7-(4,4,5,5-tetramethyl-1,3,2-dioxaborolan-2- |
| | yl)-2H-pyrido[3,2-b][1,4]oxazin-3(4H)-one |
| | MS: m/z 382.2 [M + H]⁺; RT: 0.41 min (Method 4) |
| | ¹H NMR (400 MHz, METHANOL-d₄) δ ppm 2.07-2.20 (m, 2 H), 2.44 |
| | (br d, J = 13.26 Hz, 2 H), 3.23-3.32 (m, 2 H), 3.38-3.47 (m, 1 H), 3.52 |
| | (s, 3 H), 3.57-3.65 (m, 2 H), 4.83 (s, 2 H), 7.77 (d, J = 2.25 Hz, 1 H), |
| | 8.23 (dd, J = 11.01, 1.25 Hz, 1 H), 8.29 (d, J = 1.75 Hz, 1 H), 8.43 (d, |
| | J = 2.00 Hz, 1 H), 9.00 (d, J = 1.25 Hz, 1 H) |
| 94 | Starting material: 2,7-dimethyl-5-(4,4,5,5-tetramethyl-1,3,2- |
| | dioxaborolan-2-yl)-2H-indazole |
| | MS: m/z 364.2 [M + H]⁺; RT: 0.87 min (Method 5) |
| | ¹H NMR (400 MHz, METHANOL-d₄) δ ppm 1.99-2.15 (m, 2 H), 2.31- |
| | 2.46 (m, 2 H), 2.67 (s, 3 H), 3.17-3.28 (m, 3 H), 3.53-3.61 (m, 2 H), |
| | 4.27 (s, 3 H), 7.40-7.43 (m, 1 H), 7.69-7.75 (m, 1 H), 7.86 (d, J = 1.00 |
| | Hz, 1 H), 7.98 (d, J = 2.00 Hz, 1 H), 8.29 (s, 1 H), 8.67 (d, J = 1.50 Hz, 1 H) |
| 98 | Starting material: 5-(4,4,5,5-tetramethyl-1,3,2-dioxaborolan-2- |
| | yl)benzo[d]isothiazole |
| | MS: m/z 353.2 [M + H]⁺; RT: 0.90 min (Method 5) |
| | ¹H NMR (400 MHz, METHANOL-d₄) δ ppm 1.99-2.16 (m, 2 H), 2.35 |
| | (br dd, J = 13.63, 1.88 Hz, 2 H), 3.19-3.29 (m, 3 H), 3.52-3.59 (m, 2 H), |
| | 7.71 (dd, J = 11.88, 1.38 Hz, 1 H), 7.92-7.99 (m, 2 H), 8.25 (d, J = 8.50 |
| | Hz, 1 H), 8.50 (d, J = 1.25 Hz, 1 H), 8.75 (d, J = 1.25 Hz, 1 H), 9.08 (d, |
| | J = 0.75 Hz, 1 H) |
| 97 | Starting material: 8-fluoro-2-methyl-6-(4,4,5,5-tetramethyl-1,3,2- |
| | dioxaborolan-2-yl)imidazo[1,2-a]pyridine |
| | MS: m/z 368.2 [M + H]⁺; RT: 0.76 min (Method 5) |
| | ¹H NMR (500 MHz, DMSO-d6) δ ppm 1.85-1.94 (m, 2 H) 2.15-2.21 |
| | (m, 2 H) 2.41-2.46 (m, 3 H) 3.13 (br s, 3 H) 3.35-3.41 (m, 2 H) 7.61- |
| | 7.67 (m, 1 H) 7.71-7.78 (m, 1 H) 7.89-7.95 (m, 2 H) 8.31-8.40 (m, 1 |
| | H) 8.57-8.65 (m, 1 H) 8.84-8.88 (m, 1 H) 8.89-8.93 (m, 1 H) Note: 1 |
| | extra proton from quaternary amine salt |
| 50 | Starting material: 2-methyl-6-(4,4,5,5-tetramethyl-1,3,2-dioxaborolan-2- |
| | yl)-1H-benzo[d]imidazole |
| | MS: m/z 348.2 [M + H]⁺; RT: 0.71 min (Method 5) |
| | ¹H NMR (500 MHz, DMSO-d6) δ ppm 1.84-1.94 (m, 2 H) 2.15-2.22 |
| | (m, 2 H) 2.78 (br s, 3 H) 3.03-3.15 (m, 3 H) 3.39 (br d, J = 11.67 Hz, 2 H) |
| | 7.65-7.72 (m, 1 H) 7.80-7.85 (m, 1 H) 7.85-7.90 (m, 1 H) 7.93-7.98 |
| | (m, 1 H) 8.02-8.07 (m, 1 H) 8.33-8.46 (m, 1 H) 8.59-8.68 (m, 1 H) |
| | 8.83-8.89 (m, 1 H) |
| 55 | Starting material: 1-methyl-6-(4,4,5,5-tetramethyl-1,3,2-dioxaborolan-2- |
| | yl)indolin-2-one |
| | MS: m/z 365.2 [M + H]⁺; RT: 0.55 min (Method 5) |
| | ¹H NMR (500 MHz, DMSO-d6) δ ppm 1.83-1.94 (m, 2 H) 2.14-2.21 |
| | (m, 2 H) 3.04-3.13 (m, 3 H) 3.18-3.23 (m, 3 H) 3.38 (br d, J = 12.24 Hz, |
| | 2 H) 7.35 (br d, J = 10.82 Hz, 2 H) 7.62-7.68 (m, 1 H) 7.90-7.94 (m, 1 H) |
| | 8.29-8.41 (m, 1 H) 8.56-8.66 (m, 1 H) 8.79-8.84 (m, 1 H) |
| 11 | Starting material: 2-methyl-5-(4,4,5,5-tetramethyl-1,3,2-dioxaborolan-2- |
| | yl)-2H-indazole |
| | MS: m/z 350.2 [M + H]⁺; RT: 0.72 min (Method 5) |
| | ¹H NMR (500 MHz, DMSO-d6) δ ppm 1.83-1.94 (m, 2 H) 2.15-2.21 |
| | (m, 2 H) 3.03-3.14 (m, 3 H) 3.36-3.41 (m, 2 H) 4.20 (s, 3 H) 7.56-7.61 |
| | (m, 1 H) 7.61-7.67 (m, 1 H) 7.70-7.74 (m, 1 H) 7.89-7.93 (m, 1 H) |
| | 8.03-8.07 (m, 1 H) 8.29-8.39 (m, 1 H) 8.42-8.45 (m, 1 H) 8.57-8.64 |
| | (m, 1 H) 8.76-8.81 (m, 1 H) Note: 1 extra proton from quaternary amine |
| | salt |
| 49 | Starting material: 2-(4,4,5,5-tetramethyl-1,3,2-dioxaborolan-2- |
| | yl)thieno[2,3-c]pyridine |
| | MS: m/z 353.2 [M + H]⁺; RT: 0.48 min (Method 5) |
| | ¹H NMR (500 MHz, DMSO-d6) δ ppm 1.83-1.94 (m, 2 H) 2.14-2.22 |
| | (m, 2 H) 3.03-3.16 (m, 3 H) 3.35-3.44 (m, 2 H) 7.80-7.86 (m, 1 H) |
| | 8.00-8.03 (m, 1 H) 8.05-8.09 (m, 1 H) 8.13-8.17 (m, 1 H) 8.32-8.42 |
| | (m, 1 H) 8.58-8.68 (m, 2 H) 9.08-9.12 (m, 1 H) 9.46-9.52 (m, 1 H) |
| | Note: 1 extra proton from quaternary amine salt |
| 59 | Starting material: 5-(4,4,5,5-tetramethyl-1,3,2-dioxaborolan-2-yl)-1H- |
| | indazole |
| | MS: m/z 336.2 [M + H]⁺; RT: 0.56 min (Method 5) |
| | NMR: 1H NMR (500 MHz, DMSO-d6) δ ppm 1.83-1.94 (m, 2 H) 2.15- |
| | 2.22 (m, 2 H) 3.03-3.15 (m, 3 H) 3.35-3.44 (m, 2 H) 7.62-7.71 (m, 3 |
| | H) 7.91-7.95 (m, 1 H) 8.10 (s, 1 H) 8.14-8.18 (m, 1 H) 8.30-8.39 (m, |
| | 1 H) 8.57-8.66 (m, 1 H) 8.76-8.81 (m, 1 H) 13.13-13.32 (m, 1 H) Note: |
| | 1 extra proton from quaternary amine salt |
| 56 | Starting material: 2-(4,4,5,5-tetramethyl-1,3,2-dioxaborolan-2-yl)-1H- |
| | indole-4-carbonitrile |
| | MS: m/z 360.2 [M + H]⁺; RT: 0.96 min (Method 5) |
| | ¹H NMR (500 MHz, DMSO-d6) δ ppm 1.85-1.96 (m, 2 H) 2.15-2.22 |
| | (m, 2 H) 3.03-3.14 (m, 3 H) 3.35-3.42 (m, 2 H) 7.17-7.24 (m, 1 H) |
| | 7.26-7.33 (m, 1 H) 7.54-7.60 (m, 1 H) 7.74-7.80 (m, 1 H) 7.80-7.88 |
| | (m, 1 H) 7.93-7.97 (m, 1 H) 8.29-8.42 (m, 1 H) 8.57-8.66 (m, 1 H) |
| | 9.02-9.07 (m, 1 H) 12.29-12.34 (m, 1 H) Note: 1 extra proton from |
| | quaternary amine salt |
| 48 | Starting material: 4-(4,4,5,5-tetramethyl-1,3,2-dioxaborolan-2- |
| | yl)benzo[c][1,2,5]oxadiazole |
| | MS: m/z 338.2 [M + H]⁺; RT: 0.91 min (Method 5) |
| | ¹H NMR (500 MHz, DMSO-d6) δ ppm 1.84-1.94 (m, 2 H) 2.16-2.23 |
| | (m, 2 H) 3.03-3.16 (m, 3 H) 3.38 (br d, J = 12.81 Hz, 2 H) 7.72-7.79 (m, |
| | 1 H) 7.84-7.90 (m, 1 H) 8.02 (d, J = 6.83 Hz, 1 H) 8.08-8.13 (m, 1 H) |
| | 8.15-8.19 (m, 1 H) 8.29-8.41 (m, 1 H) 8.57-8.66 (m, 1 H) 9.26-9.30 |
| | (m, 1 H) Note: 1 extra proton from quaternary amine salt |
| 53 | Starting material: 3-methyl-5-(4,4,5,5-tetramethyl-1,3,2-dioxaborolan-2- |
| | yl)-2H-indazole |
| | MS: m/z 350.2 [M + H]⁺; RT: 0.81 min (Method 5) |
| | ¹H NMR (500 MHz, DMSO-d6) δ ppm 1.83-1.94 (m, 2 H) 2.15-2.22 |
| | (m, 2 H) 2.53-2.56 (m, 3 H) 3.03-3.14 (m, 3 H) 3.36-3.43 (m, 2 H) |
| | 7.53-7.61 (m, 1 H) 7.65-7.73 (m, 2 H) 7.87-7.96 (m, 1 H) 8.04-8.10 |
| | (m, 1 H) 8.28-8.42 (m, 1 H) 8.56-8.67 (m, 1 H) 8.75-8.84 (m, 1 H) |
| | 12.69-12.88 (m, 1 H) |
| 51 | Starting material: 5-(4,4,5,5-tetramethyl-1,3,2-dioxaborolan-2- |
| | yl)benzo[c][1,2,5]oxadiazole |
| | MS: m/z 338.2 [M + H]⁺; RT: 0.61 min (Method 5) |
| | NMR: ¹H NMR (500 MHz, DMSO-d6) δ ppm 1.85-1.95 (m, 2 H) 2.15- |
| | 2.22 (m, 2 H) 3.03-3.16 (m, 3 H) 3.37 (br s, 2 H) 7.81-7.87 (m, 1 H) |
| | 7.92-7.96 (m, 1 H) 8.02-8.07 (m, 1 H) 8.21-8.26 (m, 1 H) 8.30-8.40 |
| | (m, 1 H) 8.42-8.46 (m, 1 H) 8.58-8.66 (m, 1 H) 9.06-9.09 (m, 1 H) |
| | Note: 1 extra proton from quaternary amine salt |
| 52 | Starting material: 4-fluoro-6-(4,4,5,5-tetramethyl-1,3,2-dioxaborolan-2- |
| | yl)-1H-indazole |
| | MS: m/z 354.2 [M + H]⁺; RT: 0.87 min (Method 5) |
| | ¹H NMR (400 MHz, METHANOL-d₄) δ ppm 2.00-2.14 (m, 2 H), 2.32- |
| | 2.40 (m, 2 H), 3.18-3.28 (m, 3 H), 3.51-3.60 (m, 2 H), 7.22 (dd, J = 11.26, |
| | 1.25 Hz, 1 H), 7.67-7.73 (m, 2 H), 7.99 (d, J = 2.50 Hz, 1 H), 8.18 (d, |
| | J = 1.00 Hz, 1 H), 8.74 (d, J = 1.00 Hz, 1 H) |
| 54 | Starting material: 5-(4,4,5,5-tetramethyl-1,3,2-dioxaborolan-2- |
| | yl)benzo[c][1,2,5]thiadiazole |
| | MS: m/z 354.2 [M + H]⁺; RT: 0.90 min (Method 5) |
| | ¹H NMR (400 MHz, METHANOL-d₄) δ ppm 2.00-2.15 (m, 2 H), 2.31- |
| | 2.39 (m, 2 H), 3.18-3.28 (m, 3 H), 3.51-3.60 (m, 2 H), 7.74 (dd, J = 12.01, |
| | 1.50 Hz, 1 H), 7.99 (d, J = 3.00 Hz, 1 H), 8.03-8.08 (m, 1 H), 8.14-8.19 |
| | (m, 1 H), 8.33-8.37 (m, 1 H), 8.84 (d, J = 1.00 Hz, 1 H) |
| 57 | Starting material: 5-(4,4,5,5-tetramethyl-1,3,2-dioxaborolan-2-yl)indolin- |
| | 2-one |
| | MS: m/z 369.0 [M + H]⁺; RT: 0.50 min (Method 5) |
| | ¹H NMR (400 MHz, METHANOL-d₄) δ ppm 1.99-2.13 (m, 2 H), 2.29- |
| | 2.40 (m, 2 H), 3.15-3.28 (m, 3 H), 3.50-3.60 (m, 2 H), 3.70 (s, 2 H), |
| | 7.40-7.46 (m, 2 H), 7.57-7.64 (m, 1 H), 7.95 (d, J = 3.00 Hz, 1 H), 8.62 |
| | (d, J = 1.50 Hz, 1 H) |
| 58 | Starting material: 7-(4,4,5,5-tetramethyl-1,3,2-dioxaborolan-2- |
| | yl)phthalazin-1(2H)-one |
| | MS: m/z 364.2[M + H]⁺; RT: 0.77 min (Method 5) |
| 104 | Starting material: 7-fluoro-1-methyl-5-(4,4,5,5-tetramethyl-1,3,2- |
| | dioxaborolan-2-yl)-1H-indazole |
| | MS: m/z 368.2[M + H]⁺; RT: 0.47 min (Method 4) |
| 82 | Starting material: 4-fluoro-2-methyl-6-(4,4,5,5-tetramethyl-1,3,2- |
| | dioxaborolan-2-yl)benzo[d]oxazole |
| | MS: m/z 369.2[M + H]⁺; RT: 0.47 min (Method 4) |
| 83 | Starting material: 7-methoxy-2-methyl-5-(4,4,5,5-tetramethyl-1,3,2- |
| | dioxaborolan-2-yl)-2H-indazole |
| | MS: m/z 380.2[M + H]⁺; RT: 0.41 min (Method 4) |
| 84 | Starting material: 7-(difluoromethyl)-2-methyl-5-(4,4,5,5-tetramethyl- |
| | 1,3,2-dioxaborolan-2-yl)-2H-indazole |
| | MS: m/z 400.2[M + H]⁺; RT: 0.45 min (Method 4) |
| 85 | Starting material: 2-methyl-5-(4,4,5,5-tetramethyl-1,3,2-dioxaborolan-2- |
| | yl)-2H-indazole-7-carbonitrile |
| | MS: m/z 375.2[M + H]⁺; RT: 0.42 min (Method 4) |
| 86 | Starting material: 4-fluoro-2-methyl-6-(4,4,5,5-tetramethyl-1,3,2- |
| | dioxaborolan-2-yl)-2H-indazole |
| | MS: m/z 368.2[M + H]⁺; RT: 0.45 min (Method 4) |
| 87 | Starting material: 4-fluoro-2-methyl-6-(4,4,5,5-tetramethyl-1,3,2- |
| | dioxaborolan-2-yl)-2H-benzo[d][1,2,3]triazole |
| | MS: m/z 369.2[M + H]⁺; RT: 0.45 min (Method 4) |
| 88 | Starting material: 8-fluoro-2-methyl-6-(4,4,5,5-tetramethyl-1,3,2- |
| | dioxaborolan-2-yl)-[1,2,4]triazolo[1,5-a]pyridine |
| | MS: m/z 369.2[M + H]⁺; RT: 0.38 min (Method 4) |
| 89 | Starting material: 8-fluoro-2-methyl-6-(4,4,5,5-tetramethyl-1,3,2- |
| | dioxaborolan-2-yl)-[1,2,4]triazolo[1,5-a]pyridine |
| | MS: m/z 380.2[M + H]⁺; RT: 0.36 min (Method 4) |
| 90 | Starting material: 4-fluoro-2-methyl-6-(4,4,5,5-tetramethyl-1,3,2- |
| | dioxaborolan-2-yl)benzo[d]thiazole |
| | MS: m/z 380.2[M + H]⁺; RT: 0.36 min (Method 4) |
| 164 | Starting material: 3-methoxy-6-(4,4,5,5-tetramethyl-1,3,2-dioxaborolan- |
| | 2-yl)isoquinoline |
| | MS: m/z 367.1 [M + H]⁺; RT: 0.83 min (Method 3) |
| 199 | Starting material: 2-bromo-5-(4,4,5,5-tetramethyl-1,3,2-dioxaborolan-2- |
| | yl)pyrazolo[1,5-a]pyrimidine |
| | MS: m/z 415.0 [M + H]⁺; RT: 1.35 min (Method 3) |
| 200 | Starting material: 2-methyl-5-(4,4,5,5-tetramethyl-1,3,2-dioxaborolan-2- |
| | yl)pyrazolo[1,5-a]pyrimidine |
| | MS: m/z 351.1 [M + H]⁺; RT: 1.12 min (Method 3) |
| | ¹H NMR (600 MHz, DMSO-d₆) δ ppm 1.85-1.93 (m, 2 H) 2.18 (br dd, |
| | J = 13.73, 2.29 Hz, 2 H) 2.45 (s, 3 H) 3.04-3.14 (m, 4 H) 3.40 (m, 2 H) |
| | 7.55 (d, J = 7.25 Hz, 1 H) 7.92 (dd, J = 12.40, 1.33 Hz, 1 H) 8.35 (m, 1 H) |
| | 8.62 (m, 1 H) 9.11-9.16 (m, 1 H) 9.37 (d, J = 1.53 Hz, 1 H) |
| 201 | Starting material: 6-methyl-2-(4,4,5,5-tetramethyl-1,3,2-dioxaborolan-2- |
| | yl)thieno[3,2-d]pyrimidine |
| | MS: m/z 368.1 [M + H]⁺; RT: 1.32 min (Method 3) |

### Example 6—Compound 153 and/or 166

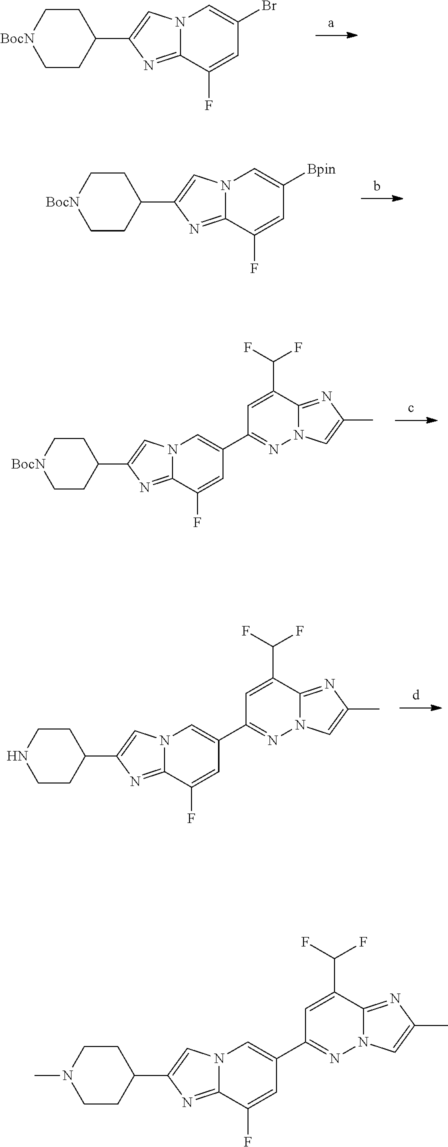

Step a: Tert-butyl 4-(6-bromo-8-fluoro-imidazo[1,2-a]pyridin-2-yl)piperidine-1-carboxylate (885.3 mg, 1.93 mmol, 1.0 eq.) was dissolved in dioxane (11.11 mL, 0.174 M) before 4,4,5,5-tetramethyl-2-(4,4,5,5-tetramethyl-1,3,2-dioxaborolan-2-yl)-1,3,2-dioxaborolane (736.64 mg, 2.90 mmol, 1.5 eq.) and potassium acetate (379.59 mg, 3.87 mmol, 2 eq.) were added. The solution was then sparged with nitrogen before Pd(dppf)Cl₂ CH₂Cl₂ (78.96 mg, 96.69 μmol 0.05 eq.) was added. The solution then stirred at 60° C. for 4 hours before being raised to 100° C. and for 16 hours. The solution was then concentrated in vacuo before being injected crude onto normal phase and purified via 0-25% MeOH:DCM. Product elutes at 15% MeOH over 12 minutes. Identified fractions were combined and concentrated to obtain [2-(1-tert-butoxycarbonyl-4-piperidyl)-8-fluoro-imidazo[1,2-a]pyridin-6-yl]boronic acid (796.8 mg, 1.82 mmol, 94% yield). MS: m/z 308.0 [M+H]⁺; RT: 0.54 min (Method 4).

Step b: 6-Chloro-8-(difluoromethyl)-2-methylimidazo[1,2-b]pyridazine (37.52 mg, 0.126 mmol, 1.1 eq.) was dissolved in Dioxane (1.5 mL, 0.08 M) and water (0.25 mL, 0.46 M) before [2-(1-tert-butoxycarbonyl-4-piperidyl)-8-fluoro-imidazo[1,2-a]pyridin-6-yl]boronic acid (50 mg, 0.114 mmol, 1.0 eq.) and potassium carbonate (111.6 mg, 0.343 mmol, 3 eq.) were added. The solution was then sparged with nitrogen before Pd(dppf)Cl₂ CH₂Cl₂ (11.1 mg, 17.14 μmol 0.15 eq.) was added. The solution then stirred at 120° C. for 16 hours before being concentrated and carried forward crude as is. Obtained tert-butyl 4-(6-(8-(difluoromethyl)-2-methylimidazo[1,2-b]pyridazin-6-yl)-8-fluoroimidazo[1,2-a]pyridin-2-yl)piperidine-1-carboxylate assumed quantitative yield (57.19 mg, 0.114 mmol, 72.3% yield) MS: m/z 501.2 [M+H]⁺ RT: 0.66 min (Method 4).

Step c: Tert-butyl 4-(6-(8-(difluoromethyl)-2-methylimidazo[1,2-b]pyridazin-6-yl)-8-fluoroimidazo[1,2-a]pyridin-2-yl)piperidine-1-carboxylate (57.19 mg, 0.114 mmol, 1.0 eq.) was dissolved in DCM (3 mL, 0.04 M) before 4 M HCl in dioxane (285 μL, 1.14 mmol, 10.0 eq.) was added. The solution then stirred at 50° C. for 2 hours before an extra aliquot of 4 M HCl in dioxane (285 μL, 0.114 mmol, 1.0 eq.) was added. The solution stirred at 50° C. for another 16 hours before being concentrated purified via preparative HPLC (Column: Sunfire C18 100×19 mm, 5 mm; Mobile phase A: MeCN; Mobile phase B: H2O; Modifier: 0.1% TFA). Obtained 8-(difluoromethyl)-6-(8-fluoro-2-(piperidin-4-yl)imidazo[1,2-a]pyridin-6-yl)-2-methylimidazo[1,2-b]pyridazine (5.4 mg, 9% yield). MS: m/z 401.3 [M+H]⁺; RT: 0.93 min (Method 3) 1H NMR (600 MHz, DMSO-d6) δ ppm 1.86-1.95 (m, 2H) 2.15-2.23 (m, 2H) 2.41-2.49 (m, 3H) 3.03-3.16 (m, 3H) 3.37-3.42 (m, 2H) 7.50-7.58 (m, 1H) 7.80-7.86 (m, 1H) 7.95-8.02 (m, 2H) 8.26-8.30 (m, 1H) 8.30-8.39 (m, 1H) 8.57-8.66 (m, 1H) 9.28-9.38 (m, 1H).

Step d: 8-(difluoromethyl)-6-[8-fluoro-2-(4-piperidyl)imidazo[1,2-a]pyridin-6-yl]-2-methyl-imidazo[1,2-b]pyridazine (17.32 mg, 38.93 umol, 1.0 eq.) was dissolved in Acetonitrile (203.31 uL, 0.2 M) before TEA (7.88 mg, 77.86 umol, 2.0 eq.) was added. The solution stirred for one minute before Paraformaldehyde (46.70 mg, 38.93 umol, 1.0 eq.) and Acetic acid (11.69 mg, 194.66 umol, 5.0 eq.) were added. The solution immediately smoked and stirred at RT for 10 minutes before Sodium cyanoborohydride (4.89 mg, 77.86 umol, 2.0 eq.) was added. The solution then stirred for 30 minutes before it was concentrated then taken back up in DMSO and filtered then injected crude onto reversed phase and purified via HPLC purification (Column: Sunfire C18 100×19 mm, 5 mm; Mobile phase A: MeCN; Mobile phase B: H2O; Modifier: 0.1% TFA). Identified fractions were collected, combined, and concentrated to yield a clear film. The material was then taken back up in a minimal amount of ACN and water then lyophilized to obtain 8-(difluoromethyl)-6-[8-fluoro-2-(1-methyl-4-piperidyl)imidazo[1,2-a]pyridin-6-yl]-2-methyl-imidazo[1,2-b]pyridazine (10 mg, 47.15% yield). as a fluffy yellow powder that was registered as is. MS: m/z 415.2 [M+H]⁺; RT: 0.46 min (Method 4). 1H NMR (400 MHz, METHANOL-d4) δ ppm 2.00-2.13 (m, 2H) 2.33-2.42 (m, 2H) 2.55-2.58 (m, 3H) 2.93-2.98 (m, 3H) 3.15-3.26 (m, 3H) 3.63-3.70 (m, 2H) 7.19-7.48 (m, 1H) 7.92-8.00 (m, 2H) 8.07-8.11 (m, 1H) 8.18-8.22 (m, 1H) 9.18-9.21 (m, 1H) Using the procedure described for Example 6 above, additional compounds described herein were prepared by substituting the appropriate heteroaryl halide starting material in step b, suitable reagents and reaction conditions, obtaining compounds such as those selected from:

| Cpd ID | Data |
|---|---|
| 81 | Starting material: 6-chloro-8-(difluoromethyl)-2-methylimidazo[1,2- |
| | b]pyridazine |
| | MS: m/z 401.3 [M + H]⁺; RT: 0.93 min (Method 3) |
| | ¹H NMR (600 MHz, DMSO-d6) δ ppm 1.86-1.95 (m, 2 H) 2.15-2.23 (m, 2 |
| | H) 2.41-2.49 (m, 3 H) 3.03-3.16 (m, 3 H) 3.37-3.42 (m, 2 H) 7.50-7.58 (m, |
| | 1 H) 7.80-7.86 (m, 1 H) 7.95-8.02 (m, 2 H) 8.26-8.30 (m, 1 H) 8.30-8.39 |
| | (m, 1 H) 8.57-8.66 (m, 1 H) 9.28-9.38 (m, 1 H) |
| 92 | Starting material: 5-chloro-2,7-dimethyl-2H-pyrazolo[3,4-c]pyridine |
| | MS: m/z 364.2 [M + H]⁺; RT: 0.63 min (Method 3) |
| | ¹H NMR (600 MHz, DMSO-d6) δ ppm 1.87-1.90 (m, 2 H) 2.16-2.20 (m, 2 |
| | H) 2.80-2.82 (m, 3 H) 3.03-3.12 (m, 5 H) 4.25-4.26 (m, 3 H) 7.85-7.89 (m, |
| | 1 H) 8.05 (d, J = 2.41 Hz, 1 H) 8.08-8.10 (m, 1 H) 8.33-8.35 (m, 1 H) 8.49- |
| | 8.52 (m, 1 H) 8.61-8.62 (m, 1 H) 9.11-9.14 (m, 1 H) |
| 101 | Starting material: 6-chloro-2,7-dimethylimidazo[1,2-b]pyridazine |
| | MS: m/z 365.2 [M + H]⁺ RT: 1.38 min (Method 3). 1H NMR (600 MHz, DMSO- |
| | d6) δ ppm 1.86-1.95 (m, 2 H) 2.15-2.22 (m, 2 H) 2.51-2.74 (m, 3 H) 2.99 |
| | (br s, 4 H) 3.33-3.43 (m, 1 H) 4.21-4.27 (m, 3 H) 7.73-7.75 (m, 1 H) 7.87- |
| | 7.92 (m, 1 H) 7.95-7.99 (m, 1 H) 8.27-8.36 (m, 1 H) 8.53-8.61 (m, 1 H) 8.62- |
| | 8.65 (m, 1 H) 9.20-9.25 (m, 1 H) |
| 102 | Starting material: 6-chloro-2,7-dimethylimidazo[1,2-b]pyridazine |
| | MS: m/z 365.1 [M + H]⁺; RT: 0.33 min (Method 4) |
| 103 | Starting material: 6-chloro-8-methylimidazo[1,2-b]pyridazine |
| | MS: m/z 351.0 [M + H]⁺; RT: 0.35 min (Method 4) |
| 105 | Starting material: 6-bromo-8-ethyl-2-methylimidazo[1,2-a]pyridine |
| | MS: m/z 378.2 [M + H]⁺; RT: 0.39 min (Method 4) |
| 124 | Starting material: 6-chloro-8-methyl-[1,2,4]triazolo[4,3-b]pyridazine |
| | MS: m/z 352.1 [M + H]⁺; RT: 0.36 min (Method 4) |
| 122 | Starting material: 2-bromo-4,6-difluorobenzo[d]oxazole |
| | MS: m/z 373.1 [M + H]⁺; RT: 0.59 min (Method 4) |
| 123 | Starting material: 2-bromo-6-fluoroimidazo[1,2-a]pyridine |
| | MS: m/z 354.1 [M + H]⁺; RT: 0.38 min (Method 4) |
| 125 | Starting material: 6-bromo-2,8-dimethyl-imidazo[1,2-a]pyrazine |
| | MS: m/z 365.3 [M + H]⁺; RT: 0.34 min (Method 4) |
| 194 | Starting material: 6-chloro-8-cyclopropyl-2-methylimidazo[1,2-b]pyridazine |
| | MS: m/z 391.1 [M + H]⁺; RT: 1.287 min (Method 10) |
| 195 | Starting material: 2-bromo-4,6-dimethylpyrazolo[1,5-a]pyrazine |
| | MS: m/z 365.1 [M + H]⁺; RT: 1.65 min (Method 10) |
| | ¹H NMR: (400 MHz, METHANOL-d₄) δ ppm = 8.85 (s, 1H), 8.56 (s, 1H), 8.25 |
| | (s, 1H), 7.86 (d, J = 3.0 Hz, 1H), 7.67-7.65 (m, 1H), 7.26 (s, 1H), 3.46-3.40 (m, |
| | 2H), 3.15-3.08 (m, 3H), 2.73 (s, 3H), 2.46 (s, 3H), 2.30-2.25 (m, 2H), 2.00-1.90 |
| | (m, 2H). |
| 197 | Starting material: 6-chloro-8-ethyl-2-methylimidazo[1,2-b]pyridazine |
| | MS: m/z 379.1 [M + H]⁺; RT: 1.58 min (Method 10) |
| | ¹H NMR: (400 MHz, METHANOL-d₄) δ ppm = 9.18 (d, J = 1.2, 1H), 8.17 (s, |
| | 1H), 7.98 (d, J = 3.2, 1H), 7.94-7.99 (m, 2H), 3.57-3.54 (m, 2H), 3.25-3.20 (m, |
| | 3H), 3.19-3.13 (m, 2H), 2.60 (s, 3H), 2.36-2.32 (m, 2H), 2.10-2.00 (m, 2H), 1.51 |
| | (t, J = 7.6, 3H). |
| 198 | Starting material: 2-chloropyrrolo[2,1-f][1,2,4]triazine |
| | MS: m/z 337.1 [M + H]⁺; RT: 1.24 min (Method 3) |
| 224 | Starting material: 6-chloro-2,8-dimethyl-[1,2,4]triazolo[1,5-b]pyridazine |
| | MS: m/z 380.2 [M + H]⁺; RT: 1.363 min (Method 8) |
| | ¹H NMR: (400 MHz, METHANOL-d₄) δ ppm = 2.02-2.05 (m, 2H), 2.29-2.32 |
| | (m, 2H), 2.64 (s, 3H), 2.74 (s, 3H), 2.78 (s, 3H), 2.90-2.96 (m, 2H), 3.00-3.10 |
| | (m, 1H), 3.44-3.46 (m, 2H), 7.88 (d, J = 9.6 Hz, 1H), 7.95 (d, J = 2.4 Hz, 1H), |
| | 7.99 (s, 1H), 8.51 (s, 1H), 9.11 (s, 1H). |
| 225 | Starting material: 6-chloro-8-methoxy-2-methyl-[1,2,4]triazolo[1,5- |
| | b]pyridazine |
| | MS: m/z 396.1 [M + H]⁺; RT: 1.353 min (Method 10) |
| | ¹H NMR: (400 MHz, METHANOL-d₄) δ ppm = 2.03-2.07 (m, 2H), 2.30-2.40 |
| | (m, 2H), 2.60 (s, 3H), 2.81 (s, 3H), 2.98-3.01(m, 2H), 3.07-3.13 (m, 1H), 3.50- |
| | 3.60 (m, 2H), 4.28 (s, 3H), 7.53 (s, 1H), 7.91 (d, J = 12.0 Hz, 1H), 7.94 (d, J = |
| | 2.8 Hz, 1H), 8.51 (s, 1H), 9.15 (s, 1H). |
| 233 | Starting material: 6-chloro-8-(difluoromethyl)-2-methylimidazo[1,2- |
| | b]pyridazine |
| | MS: m/z 413.2 [M + H]⁺; RT: 1.487 min (Method 8) |
| | ¹H NMR: (400 MHz, METHANOL-d₄) δ ppm = 2.02 (s, 2H), 2.38-2.40 (m, |
| | 1H), 2.41 (s, 3H), 2.52 (s, 3H), 2.60 (d, J = 8.8 Hz, 2H), 3.22 (d, J = 9.6 Hz, 2H), |
| | 7.16-7.44 (m, 1H), 7.79-7.84 (m, 2H), 7.94 (s, 1H), 8.11 (s, 1H), 9.07 (s, 1H). |
| 243 | Starting material: 8-methoxy-2-methyl-6-(4,4,5,5-tetramethyl-1,3,2- |
| | dioxaborolan-2-yl)-[1,2,4]triazolo[1,5-b]pyridazine |
| | MS: m/z 382.3 [M + H]⁺; RT: 0.43 min (Method 4) |
| 244 | Starting material: tert-butyl 5-(2-bromoacetyl)-2-azabicyclo[2.2.1]heptane-2- |
| | carboxylate and intermediate X |
| | MS: m/z 380.3 [M + H]⁺; RT: 0.34 min (Method 4) |
| 560 | Starting material: 2-(2-azabicyclo[2.2.1]heptan-5-yl)-8-fluoro-6-(8-fluoro-2- |
| | methyl-imidazo[1,2-a]pyridin-6-yl)imidazo[1,2-a]pyridine and |
| | paraformaldehyde |
| | MS: m/z 394.2[M + H]+; RT: 0.37 min (Method 4) |
| 577 | Starting material: 6-chloro-2-methyl-[1,2,4]triazolo[1,5-b]pyridazine |

### Example 7—Compound 4

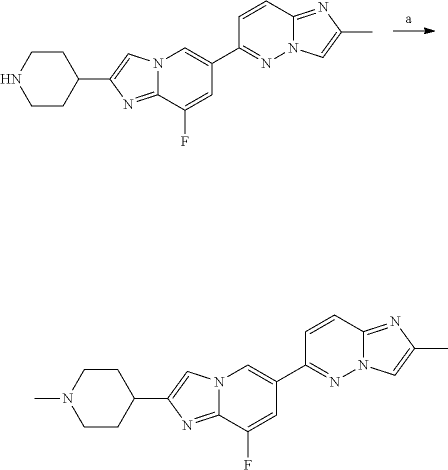

6-[8-fluoro-2-(4-piperidyl)imidazo[1,2-a]pyridin-6-yl]-2-methyl-imidazo[1,2-b]pyridazine (29.85 mg, 67.13 μmol, 1.0 eq.) was dissolved in DCM (370 μL, 0.2 M) and acetic acid (4 μL, 67.13 μmol, 1 eq.) was then added, followed by paraformaldehyde (80.5 mg, 67.13 μmol, 1.0 eq.). The solution then stirred for 5 minutes before sodium cyanoborohydride (4.2 mg, 67.1 μmol, 1.0 eq.) was added. The solution then stirred for 4 hours at rt before it was concentrated then taken back up in DMSO, filtered, and submitted to HPLC purification (Column: Sunfire C18 100×19 mm, 5 mm; Mobile phase A: MeCN; Mobile phase B: H₂O; Modifier: 0.1% TFA). Obtained 6-[8-fluoro-2-(1-methyl-4-piperidyl)imidazo[1,2-a]pyridin-6-yl]-2-methyl-imidazo[1,2-b]pyridazine (8.8 mg, 27% yield) as a yellow solid. MS: m/z 365.3 [M+H]⁺ RT: 0.70 m (Method 4). 1H NMR (600 MHz, DMSO-d6) δ ppm 1.88-1.97 (m, 2H) 2.23-2.29 (m, 2H) 2.49 (q, J=1.65 Hz, 3H) 2.82-2.88 (m, 3H) 2.99-3.06 (m, 1H) 3.08-3.18 (m, 2H) 3.52-3.60 (m, 2H) 7.73-7.76 (m, 1H) 7.77-7.81 (m, 1H) 7.98-8.01 (m, 1H) 8.10-8.13 (m, 1H) 8.14-8.18 (m, 1H) 0.9-9.23 (m, 1H)

Using the procedure described for Example 7 above, additional compounds described herein were prepared by substituting the appropriate starting material in step a, suitable reagents and reaction conditions, obtaining compounds such as those selected from:

| Cpd ID | Data |
|---|---|
| 26 | Starting material: 7-fluoro-5-(8-fluoro-2-(piperidin-4-yl)imidazo[1,2- |
| | a]pyridin-6-yl)-2-methyl-2H-indazole |
| | MS: m/z 382.3 [M + H]+; RT: 0.95 min (Method 4) |
| | ¹H NMR (600 MHz, DMSO-d6) δ ppm 1.85-1.95 (m, 2 H) 2.22-2.28 (m, |
| | 2 H) 2.80-2.89 (m, 3 H) 2.98-3.05 (m, 1 H) 3.08-3.16 (m, 2 H) 3.52-3.58 |
| | (m, 2 H) 4.21-4.27 (m, 3 H) 7.43-7.49 (m, 1 H) 7.63-7.68 (m, 1 H) 7.85- |
| | 7.89 (m, 1 H) 7.89-7.94 (m, 1 H) 8.55-8.59 (m, 1 H) 8.79-8.85 (m, 1 H) |
| | 9.30-9.43 (m, 1 H) |
| 9 | Starting material: 8-fluoro-2′,8′-dimethyl-2-(piperidin-4-yl)-6,6′- |
| | biimidazo[1,2-a]pyridine |
| | MS: m/z 378.3 [M + H]⁺; RT: 1.60 min (Method 2) |
| | ¹H NMR (600 MHz, DMSO-d6) δ ppm 1.85-1.97 (m, 2 H) 2.21-2.28 (m, |
| | 2 H) 2.54-2.56 (m, 3 H) 2.58-2.66 (m, 3 H) 2.80-2.89 (m, 3 H) 2.99-3.07 |
| | (m, 1 H) 3.11-3.15 (m, 2 H) 3.54-3.57 (m, 2 H) 7.62-7.66 (m, 1 H) 7.94- |
| | 7.98 (m, 1 H) 7.98-8.03 (m, 1 H) 8.88-8.92 (m, 1 H) 9.04-9.09 (m, 1 H) |
| | 9.43-9.48 (m, 1 H) |
| 28 | Starting material: 5-(8-fluoro-2-(piperidin-4-yl)imidazo[1,2-a]pyridin-6-yl)- |
| | 2-methyl-2H-indazole |
| | MS: m/z 364.1 [M + H]⁺; RT: 0.96 min (Method 4) |
| 6 | Starting material: 5-(8-fluoro-2-(piperidin-4-yl)imidazo[1,2-a]pyridin-6-yl)- |
| | 2,7-dimethyl-2H-indazole |
| | MS: m/z 378.4 [M + H]+; RT: 0.70 min (Method 4) |
| | ¹H NMR (600 MHz, DMSO-d6) δ ppm 1.84-1.94 (m, 2 H) 2.22-2.28 (m, |
| | 2 H) 2.80-2.87 (m, 3 H) 2.97-3.03 (m, 1 H) 3.08-3.17 (m, 2 H) 3.52-3.58 |
| | (m, 2 H) 4.18-4.24 (m, 3 H) 7.35-7.39 (m, 1 H) 7.57-7.62 (m, 1 H) 7.83- |
| | 7.86 (m, 1 H) 7.88-7.91 (m, 1 H) 8.38-8.41 (m, 1 H) 8.74-8.77 (m, 1 H) |
| 10 | Starting material: 8-fluoro-2′-methyl-2-(piperidin-4-yl)-6,6′-biimidazo[1,2- |
| | a]pyridine |
| | MS: m/z 364.1 [M + H]⁺; RT: 0.68 min (Method 4) |
| 219 | Starting material: 6-(8-fluoro-2-((1R,5S,6r)-3-methyl-3- |
| | azabicyclo[3.1.0]hexan-6-yl)imidazo[1,2-a]pyridin-6-yl)-2-methyl- |
| | [1,2,4]triazolo[1,5-b]pyridazine |
| | MS: m/z 364.0 [M + H]⁺; RT: 0.753 min (Method 10) |
| 220 | Starting material: 2-(8-fluoro-2-(piperidin-4-yl)imidazo[1,2-a]pyridin-6-yl)- |
| | 4,6-dimethylpyrazolo[1,5-a]pyrazine |
| | MS: m/z 379.2 [M + H]⁺; RT: 1.73 min (Method 10) |
| | ¹H NMR: (400 MHz, METHANOL-d₄) δ ppm = 1.83-1.96 (m, 2H), 2.20- |
| | 2.25 (m, 2H), 2.45-2.55 (m, 8H), 2.76 (s, 3H), 2.84-2.94 (m, 1H), 3.20- |
| | 3.25 (m, 2H), 7.33 (s, 1H), 7.79 (d, J = 11.6 Hz, 1H), 7.85 (d, J = 2.8 Hz, 1H), |
| | 8.31 (s, 1H), 8.91 (d, J = 1.1 Hz, 1H). |
| 222 | Starting material: 2-(2-((1R,5S,6r)-3-azabicyclo[3.1.0]hexan-6-yl)-8- |
| | fluoroimidazo[1,2-a]pyridin-6-yl)-4,6-dimethylpyrazolo[1,5-a]pyrazine |
| | MS: m/z 377.1 [M + H]⁺; RT: 1.938 min (Method 8) |
| | ¹H NMR: (400 MHz, METHANOL-d₄) δ ppm = 2.00 (s, 2H), 2.36-2.39 (m, |
| | 1H), 2.39 (s, 3H), 2.48 (s, 3H), 2.55-2.60 (m, 2H), 2.75 (s, 3H), 3.20 (d, J = |
| | 9.6 Hz, 2H), 7.30 (s, 1H), 7.64 (d, J = 12.0 Hz, 1H), 7.77 (d, J = 3.2 Hz, 1H), |
| | 8.29 (s, 1H), 8.84 (s, 1H). |
| 223 | Starting material: 5-(2-((1R,5S,6r)-3-azabicyclo[3.1.0]hexan-6-yl)-8- |
| | fluoroimidazo[1,2-a]pyridin-6-yl)-2,7-dimethylpyrazolo[1,5-a]pyrimidine |
| | MS: m/z 377.2 [M + H]⁺; RT: 1.737 min (Method 10) |
| 227 | Starting material: 6-(2-((1R,5S,6r)-3-azabicyclo[3.1.0]hexan-6-yl)-8- |
| | fluoroimidazo[1,2-a]pyridin-6-yl)-2,8-dimethyl-[1,2,4]triazolo[1,5- |
| | b]pyridazine |
| | MS: m/z 378.2 [M + H]⁺; RT: 0.966 min (Method 10) |
| | ¹H NMR: (400 MHz, METHANOL-d₄) δ ppm = 2.04 (s, 2H), 2.38 (s, 1H), |
| | 2.45 (s, 3H), 2.61 (s, 3H), 2.68 (d, J = 9.2 Hz, 2H), 2.72 (s, 3H), 3.26 (d, J = |
| | 9.6 Hz, 2H), 7.77 (d, J = 12.0 Hz, 1H), 7.83 (d, J = 2.4 Hz, 1H), 7.95 (s, 1H), |
| | 9.01 (s, 1H). |
| 228 | Starting material: 6-(2-((1R,5S,6r)-3-azabicyclo[3.1.0]hexan-6-yl)-8- |
| | fluoroimidazo[1,2-a]pyridin-6-yl)-2-(difluoromethyl)-8-methylimidazo[1,2- |
| | b]pyridazine |
| | MS: m/z 413.0 [M + H]⁺; RT: 0.921 min (Method 10) |
| 230 | Starting material: 6-(2-((1R,5S,6r)-3-azabicyclo[3.1.0]hexan-6-yl)-8- |
| | fluoroimidazo[1,2-a]pyridin-6-yl)-2-methyl-8- |
| | (trifluoromethyl)imidazo[1,2-b]pyridazine |
| | MS: m/z 431.2 [M + H]⁺; RT: 2.002 min (Method 8) |
| | ¹H NMR: (400 MHz, METHANOL-d₄) δ ppm = 2.14 (s, 2H), 2.39 (s, 1H), |
| | 2.53 (s, 3H), 2.58 (s, 3H), 2.90 (d, J = 9.8 Hz, 2H), 3.39 (d, J = 10.3 Hz, 2H), |
| | 7.82 (d, J = 12.0 Hz, 1H), 7.85 (d, J = 2.7 Hz, 1H), 8.07 (s, 1H), 8.16 (s, 1H), |
| | 9.10 (s, 1H). |

### Example 8—Compound 69

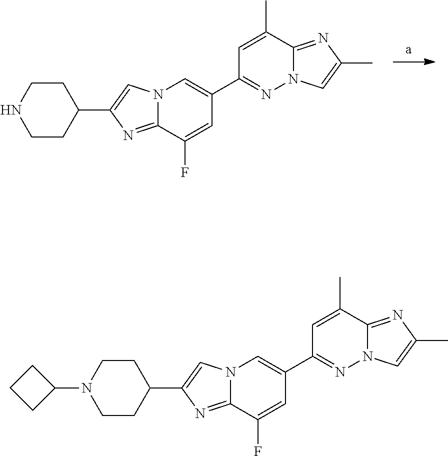 [SMILES: Cc1cn2nc(-c3cc(F)c4nc(C5CCNCC5)cn4c3)cc(C)c2n1 ; Cc1cn2nc(-c3cc(F)c4nc(C5CCN(C6CCC6)CC5)cn4c3)cc(C)c2n1]

To a solution of 6-[8-fluoro-2-(4-piperidyl)imidazo[1,2-a]pyridin-6-yl]-2,8-dimethyl-imidazo[1,2-b]pyridazine (20 mg, 54.88 μmol) in EtOH (1 mL) and DCE (0.2 mL) was added cyclobutanone (5.7 mg, 82.3 μmol, 6 μL) and TEA (109.7 μmol, 15 μL) and the mixture was stirred at 25° C. for 10 min. Then sodium triacetoxyborohydride (23 mg, 109.7 μmol) was added and the mixture was stirred at 25° C. for 0.5 h. The mixture was filtered, and the filtrate was concentrated in vacuo to give the residue, which was purified by preparative HPLC (Boston Prime C18 150*30 mm*5 um; water NH₃H₂O+NH₄HCO₃)-ACN) to give 6-[2-(1-cyclobutyl-4-piperidyl)-8-fluoro-imidazo[1,2-a]pyridin-6-yl]-2,8-dimethyl-imidazo[1,2-b]pyridazine (14.5 mg, 63% yield) as a white solid. MS: m/z 419.3 [M+H]⁺; 1H NMR (500 MHz, METHANOL-d4) δ ppm=8.99 (s, 1H), 7.92 (s, 1H), 7.83 (d, J=2.59 Hz, 3H), 7.78 (d, J=11.90 Hz, 1H), 7.57 (s, 1H), 3.02 (d, J=11.60 Hz, 2H), 2.88-2.76 (m, 2H), 2.67 (s, 3H), 2.48 (s, 3H), 2.21-2.08 (m, 4H), 2.04-1.90 (m, 4H), 1.82-1.72 (m, 4H).

Using the procedure described for Example 8 above, additional compounds described herein were prepared by substituting the appropriate amine and carbonyl starting materials in step a, suitable reagents and reaction conditions, obtaining compounds such as those selected from:

| Cpd ID | Data |
|---|---|
| 7 | Starting materials: 5-(8-fluoro-2-(piperidin-4-yl)imidazo[1,2-a]pyridin-6- |
| | yl)-2-methyl-2H-indazole and acetaldehyde |
| | MS: m/z 378.3 [M + H]⁺; RT: 0.87 min (Method 4) |
| | 1H NMR (400 MHz, METHANOL-d4) δ ppm 1.37-1.44 (m, 3 H) 2.02- |
| | 2.14 (m, 2 H) 2.38-2.46 (m, 2 H) 3.10-3.20 (m, 2 H) 3.22-3.29 (m, 3 H) |
| | 3.70-3.79 (m, 2 H) 4.24-4.27 (m, 3 H) 7.62-7.66 (m, 1 H) 7.71-7.78 (m, |
| | 2 H) 7.97-8.00 (m, 1 H) 8.02-8.05 (m, 1 H) 8.29-8.32 (m, 1 H) 8.66-8.69 |
| | (m, 1 H) |
| 25 | Starting materials: 5-(8-fluoro-2-(piperidin-4-yl)imidazo[1,2-a]pyridin-6- |
| | yl)-2,7-dimethyl-2H-indazole and acetaldehyde |
| | MS: m/z 392.4 [M + H]⁺; RT: 0.99 min (Method 4) |
| | 1H NMR (600 MHz, DMSO-d6) δ ppm 1.23-1.30 (m, 3 H) 1.86-1.96 (m, |
| | 2 H) 2.23-2.29 (m, 2 H) 2.56-2.60 (m, 3 H) 3.00-3.11 (m, 3 H) 3.14-3.20 |
| | (m, 2 H) 3.57-3.63 (m, 2 H) 4.17-4.23 (m, 3 H) 7.36-7.40 (m, 1 H) 7.56- |
| | 7.63 (m, 1 H) 7.82-7.86 (m, 1 H) 7.89-7.93 (m, 1 H) 8.38-8.42 (m, 1 H) |
| | 8.74-8.78 (m, 1 H) |
| 5 | Starting materials: 7-fluoro-5-(8-fluoro-2-(piperidin-4-yl)imidazo[1,2- |
| | a]pyridin-6-yl)-2-methyl-2H-indazole and acetaldehyde |
| | MS: m/z 396.4 [M + H]⁺; RT: 0.98 min (Method 4) |
| | 1H NMR (400 MHz, METHANOL-d4) δ ppm 1.99-2.12 (m, 3 H) 2.35- |
| | 2.42 (m, 2 H) 3.10-3.20 (m, 4 H) 3.24-3.28 (m, 2 H) 3.46-3.51 (m, 1 H) |
| | 3.69-3.76 (m, 2 H) 4.26-4.28 (m, 3 H) 7.34-7.39 (m, 1 H) 7.58-7.62 (m, |
| | 1 H) 7.82-7.84 (m, 1 H) 7.89-7.92 (m, 1 H) 8.38-8.40 (m, 1 H) 8.61-8.64 |
| | (m, 1 H) |
| 3 | Starting materials: 6-(8-fluoro-2-(piperidin-4-yl)imidazo[1,2-a]pyridin-6- |
| | yl)-2-methylimidazo[1,2-b]pyridazine and acetaldehyde |
| | MS: m/z 379.4 [M + H]⁺; RT: 0.77 min (Method 4) |
| | 1H NMR (400 MHz, METHANOL-d4) δ ppm 1.38-1.44 (m, 3 H) 2.02- |
| | 2.13 (m, 2 H) 2.35-2.43 (m, 2 H) 2.58-2.60 (m, 3 H) 3.13-3.20 (m, 2 H) |
| | 3.23-3.29 (m, 2 H) 3.45-3.52 (m, 1 H) 3.70-3.77 (m, 2 H) 7.89-7.93 (m, |
| | 1 H) 7.97-8.00 (m, 1 H) 8.10-8.14 (m, 1 H) 8.21-8.23 (m, 1 H) 8.25-8.30 |
| | (m, 1 H) 9.16-9.18 (m, 1 H) |
| 27 | Starting materials: 8-fluoro-2′,8′- |
| | dimethyl-2-(piperidin-4-yl)-6,6′-biimidazo[1,2-a]pyridine and |
| | acetaldehyde |
| | MS: m/z 392.1 [M + H]⁺; RT: 0.77 min (Method 4) |
| | 1H NMR (600 MHz, DMSO-d6) δ ppm 1.24-1.29 (m, 3 H) 1.88-1.96 (m, |
| | 2 H) 2.23-2.29 (m, 2 H) 2.52-2.56 (m, 3 H) 2.59-2.63 (m, 3 H) 3.05-3.11 |
| | (m, 3 H) 3.15-3.20 (m, 2 H) 3.58-3.63 (m, 2 H) 7.61-7.66 (m, 1 H) 7.93- |
| | 7.96 (m, 1 H) 7.97-8.03 (m, 1 H) 8.89-8.92 (m, 1 H) 9.00-9.11 (m, 1 H) |
| | 9.19-9.28 (m, 1 H) |
| 8 | Starting material: 8-fluoro-2′-methyl-2-(piperidin-4-yl)-6,6′-biimidazo[1,2- |
| | a]pyridine and acetaldehyde |
| | MS: m/z 378.4 [M + H]⁺; RT: 0.71 min (Method 4) |
| | 1H NMR (400 MHz, METHANOL-d4) δ ppm 1.38-1.43 (m, 3 H) 2.02- |
| | 2.13 (m, 2 H) 2.34-2.42 (m, 2 H) 2.58-2.60 (m, 3 H) 3.12-3.18 (m, 2 H) |
| | 3.23-3.28 (m, 2 H) 3.47-3.53 (m, 1 H) 3.70-3.76 (m, 2 H) 7.55-7.59 (m, |
| | 1 H) 7.92-7.96 (m, 2 H) 7.99-8.01 (m, 1 H) 8.20-8.23 (m, 1 H) 8.74-8.77 |
| | (m, 1 H) 9.06-9.08 (m, 1 H) |
| 111 | Starting materials: 6-(8-fluoro-2-(piperidin-4-yl)imidazo[1,2-a]pyridin-6- |
| | yl)-2,8-dimethylimidazo[1,2-b]pyridazine and paraformaldehyde |
| | MS: m/z 379.1 [M + H]⁺; RT: 1.55 min (Method 7) |
| 112 | Starting materials: 6-(8-fluoro-2-(piperidin-4-yl)imidazo[1,2-a]pyridin-6- |
| | yl)-2,8-dimethylimidazo[1,2-b]pyridazine and acetaldehyde |
| | MS: m/z 393.2 [M + H]⁺; RT: 1.91 min (Method 7) |
| 99 | Starting materials: 6-(8-fluoro-2-(piperidin-4-yl)imidazo[1,2-a]pyridin-6- |
| | yl)-2,8-dimethylimidazo[1,2-b]pyridazine and oxetan-3-one |
| | MS: m/z 421.2 [M + H]⁺; RT: 1.49 min (Method 7) |
| 210 | Starting material: 2,2-dimethyloxetan-3-one |
| | MS: m/z 449.3 [M + H]⁺; RT: 0.88 min (Method 3) |
| 211 | Starting material: 6-(2-((1R,5S,6s)-3-azabicyclo[3.1.0]hexan-6-yl)-8- |
| | fluoroimidazo[1,2-a]pyridin-6-yl)-2,8-dimethylimidazo[1,2-b]pyridazine |
| | MS: m/z 417.2 [M + H]⁺; RT: 1.340 min (Method 10) |
| | ¹H NMR (400 MHz, METHANOL-d₄) δ ppm = 1.69-1.79 (m, 2H), 1.94- |
| | 2.05 (m, 6H), 2.35-2.40 (m, 1H), 2.45 (s, 3H), 2.50-2.55 (m, 2H), 2.64 (s, |
| | 3H), 3.07-3.14 (m, 3H), 7.50 (s, 1H), 7.64-7.71 (m, 1H), 7.75 (d, J = 2.8 |
| | Hz, 1H), 7.87 (s, 1H), 8.87 (d, J = 1.2 Hz, 1H). |
| 213 | Starting material: 2-methyloxetan-3-one |
| | MS: m/z 435.3 [M + H]⁺; RT: 0.84 min (Method 3) |
| 214 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | MS: m/z 405.2 [M + H]⁺; RT: 1.343 min (Method 8) |
| 218 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and 3,3-difluorocyclobutan-1-one |
| | MS: m/z 453.3 [M + H]⁺; RT: 1.420 min (Method 10) |

### Example 9—Compound 46

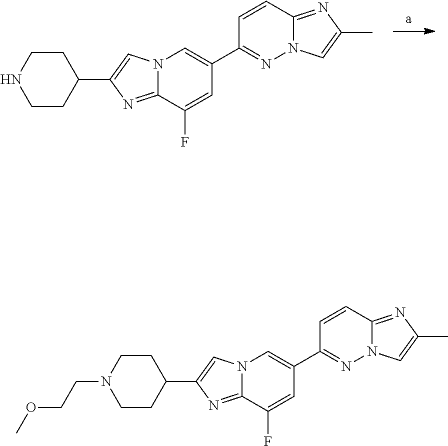

A solution of 6-[8-fluoro-2-(4-piperidyl)imidazo[1,2-a]pyridin-6-yl]-2,8-dimethyl-imidazo[1,2-b]pyridazine (30 mg, 82.32 μmol), 1-bromo-2-methoxy-ethane (11.4 mg, 82.32 μmol, 8 μL) and Cs₂CO₃ (53.6 mg, 164 μmol) in DMF (2 mL) was stirred at 80° C. for 2 hours.

The mixture was filtered and concentrated to give crude product. The crude material was purified by prep-HPLC (Boston Prime C18 150*30 mm*5 um, water (NH₃H₂O+NH₄HCO₃)-ACN as a mobile phase, from 32% to 62%, Gradient Time (min): 10, Flow Rate (ml/min): 25) to give 6-[8-fluoro-2-[1-(2-methoxyethyl)-4-piperidyl]imidazo[1,2-a]pyridin-6-yl]-2,8-dimethyl-imidazo[1,2-b]pyridazine (14.7 mg, 42% yield) as a white solid. MS: m/z 423.2 [M+H]⁺; 1H NMR: (400 MHz, METHANOL-d4) δ ppm=9.01-8.95 (m, 1H), 7.92 (s, 1H), 7.85-7.72 (m, 2H), 7.56 (s, 7H), 3.62-3.54 (s, 2H), 3.362 (s, 3H), 3.17-3.06 (m, 2H), 2.83-2.75 (m, 1H), 2.69-2.62 (m, 5H), 2.48 (s, 3H), 2.32-2.23 (m, 2H), 2.20-2.09 (m, 2H), 1.91-1.74 (m, 2H).

Using the procedure described for Example 9 above, additional compounds described herein were prepared by substituting the appropriate alkyl halide starting material in step a, suitable reagents and reaction conditions, obtaining compounds such as those selected from:

| Cpd ID | Data |
|---|---|
| 45 | Starting material: 1-bromo-3-methoxy-propane |
| | MS: m/z 437.2 [M + H]⁺ |
| | ¹H NMR: (400 MHz, METHANOL-d4) □ ppm = 9.02-9.94 (m, 1H), 7.92 (s, |
| | 1H), 7.85-7.74 (m, 2H), 7.569 (s, 1H), 3.48-3.43 (m, 2H), 3.35-3.33 (m, 3H), |
| | 3.13-3.05 (m, 2H), 2.84-2.77 (m, 1H), 2.67 (s, 3H), 2.54-2.47 (m, 5H), 2.25- |
| | 2.13 (m, 4H), 1.88-1.77 (m, 4H). |
| 47 | Starting material: 2-iodopropane |
| | MS: m/z 407.2 [M + H]⁺ |
| | ¹H NMR: (400 MHz, METHANOL-d4) □ ppm = 9.06-8.99 (m, 1H), 7.947 |
| | (s, 1H), 7.89-7.77 (m, 2H), 7.602 (s, 1H), 3.17-3.08 (m, 2H), 2.95-2.76 (m, |
| | 2H), 2.682 (s, 3H), 2.57-2.46 (m, 5H), 2.26-2.18 (m, 2H), 1.92-1.75 (m, 2H), |
| | 1.26-1.09 (m, 6H). |
| 165 | Starting material: cyclopropanecarbaldehyde |
| | MS: m/z 419.3 [M + H]⁺RT: 0.42 min (Method 3) |
| | 1H NMR (400 MHz, METHANOL-d4) δ ppm 0.17-0.22 (m, 2 H) 0.56- |
| | 0.62 (m, 2 H) 0.91-1.01 (m, 1 H) 1.80-1.91 (m, 2 H) 2.15-2.22 (m, 2 H) |
| | 2.24-2.33 (m, 2 H) 2.35-2.39 (m, 2 H) 2.47-2.50 (m, 3 H) 2.66-2.69 (m, |
| | 3 H) 2.79-2.87 (m, 1 H) 3.22-3.28 (m, 2 H) 7.57-7.60 (m, 1 H) 7.77-7.81 |
| | (m, 1 H) 7.84-7.87 (m, 1 H) 7.92-7.95 (m, 1 H) 8.99-9.02 (m, 1 H) |
| 173 | Starting material: acetaldehyde |
| | MS: m/z 393.3 [M + H]⁺RT: 1.73 min (Method 1) |
| | 1H NMR (600 MHz, DMSO-d6) δ ppm 1.22-1.29 (m, 3 H) 1.87-1.97 (m, |
| | 2 H) 2.22-2.29 (m, 2 H) 2.61-2.67 (m, 3 H) 3.02-3.11 (m, 3 H) 3.13-3.21 |
| | (m, 2 H) 3.58-3.65 (m, 2 H) 4.19-4.28 (m, 3 H) 7.72-7.76 (m, 1 H) 7.87- |
| | 7.94 (m, 1 H) 7.96-7.99 (m, 1 H) 8.60-8.65 (m, 1 H) 9.07-9.14 (m, 1 H) |
| | 9.20-9.26 (m, 1 H) |
| 174 | Starting material : 2-bromo-1,1-difluoroethane |
| | MS: m/z 429.3 [M + H]⁺ RT: 0.83 min (Method 3) |
| 175 | Starting material: acetaldehyde |
| | MS: m/z 406.3 [M + H]⁺RT: 0.81 min (Method 3) |
| | 1H NMR (600 MHz, DMSO-d6) δ ppm 1.22-1.30 (m, 3 H) 1.36 (br s, 3 H) |
| | 1.89-1.97 (m, 2 H) 2.23-2.29 (m, 2 H) 2.42 (br s, 3 H) 3.02-3.11 (m, 5 H) |
| | 3.14-3.19 (m, 2 H) 3.59-3.63 (m, 2 H) 7.67-7.76 (m, 1 H) 7.87-7.95 (m, |
| | 1 H) 8.16-8.31 (m, 1 H) 8.34-8.53 (m, 1 H) 8.90-8.99 (m, 1 H) 9.15-9.28 |
| | (m, 1 H) |
| 187 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and 1-bromo-2-methoxyethane |
| | MS: m/z 421.2 [M + H]⁺; RT: 1.645 min (Method 10) |
| | ¹H NMR (500 MHz, CHLOROFORM-d) δ ppm = 2.30 (s, 2H), 2.53 (s, 3H), |
| | 2.65 (s, 1H), 2.71 (s, 3H), 3.11 (d, J = 10.5 Hz, 2H), 3.16 (t, J = 4.5 Hz, 2H), |
| | 3.37 (s, 3H), 3.63-3.74 (m, 2H), 3.80-3.85 (m, 2H), 7.15 (s, 1H), 7.51 (d, |
| | J = 11.5 Hz, 1H), 7.62 (d, J = 2.5 Hz, 1H), 7.74 (s, 1H), 8.50 (s, 1H). |
| 188 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and acetaldehyde |
| | MS: m/z 391.1 [M + H]⁺; RT: 1.214 min (Method 8) |
| 192 | Starting material: 6-(8-fluoro-2-(piperidin-4-yl)imidazo[1,2-a]pyridin-6-yl)- |
| | 2,8-dimethylimidazo[1,2-b]pyridazine and dihydrofuran-3(2H)-one |
| | MS: m/z 435.2 [M + H]⁺; RT: 1.557 min (Method 10) |
| 212 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and cyclopropanecarbaldehyde |
| | MS: m/z 417.3 [M + H]⁺; RT: 1.377 min (Method 10) |
| | ¹H NMR: (400 MHz, METHANOL-d₄) δ ppm = 0.30-0.40 (m, 2H), 0.70- |
| | 0.80 (m, 2H), 1.09-1.10 (m, 1H), 2.05-2.20 (m, 1H), 2.25-2.35 (m, 2H), |
| | 2.47 (s, 3H), 2.65 (s, 3H), 2.94 (s, 2H), 3.37 (s, 2H), 3.66-3.68 (m, 2H), 7.53 |
| | (s, 1H), 7.76 (d, J = 12.0 Hz, 1H), 7.89 (s, 1H), 8.52 (s, 1H), 8.94 (s, 1H). |
| 216 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and 1-fluoro-2-iodoethane |
| | MS: m/z 409.1 [M + H]⁺; RT: 0.672 min (Method 10) |
| 217 | Starting material: 6-(2-((1R,5S,6r)-3-azabicyclo[3.1.0]hexan-6-yl)-8- |
| | fluoroimidazo[1,2-a]pyridin-6-yl)-2,8-dimethylimidazo[1,2-b]pyridazine |
| | and 1,1-difluoro-2-iodoethane |
| | MS: m/z 427.2 [M + H]⁺; RT: 1.303. min (Method 10) |
| 221 | Starting material: 6-(8-fluoro-2-(piperidin-4-yl)imidazo[1,2-a]pyridin-6-yl)- |
| | 2,4-dimethylpyrrolo[2,1-f][1,2,4]triazine and 1-bromo-2-methoxyethane |
| | MS: m/z 423.2 [M + H]⁺; RT: 1.77 min (Method 10) |
| | ¹H NMR: (400 MHz, METHANOL-d₄) δ ppm = 1.81-1.95 (m, 2H), 2.18 (d, |
| | J = 12.8 Hz, 2H), 2.39-2.51 (m, 5H), 2.76 (s, 3H), 2.77-2.95 (m, 3H), 3.21 |
| | (d, J = 10.8 Hz, 2H), 3.38 (s, 3H), 3.60-3.63 (m, 2H), 7.32 (s, 1H), 7.67- |
| | 7.70 (m, 1H), 7.83 (d, J = 2.8 Hz, 1H), 8.30 (s, 1H), 8.90 (s, 1H). |

### Example 10—Compound 74

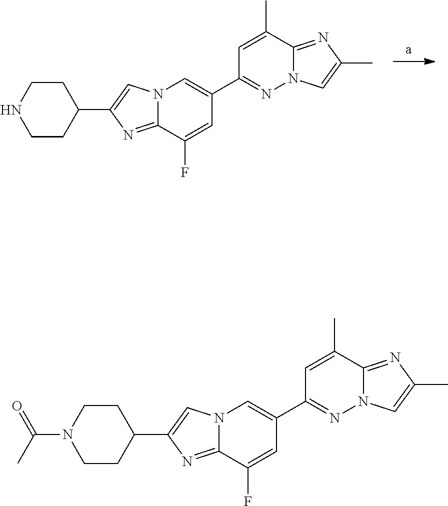 [SMILES: Cc1cn2nc(-c3cc(F)c4nc(C5CCNCC5)cn4c3)cc(C)c2n1 ; CC(=O)N1CCC(c2cn3cc(-c4cc(C)c5nc(C)cn5n4)cc(F)c3n2)CC1]

A solution of 6-[8-fluoro-2-(4-piperidyl)imidazo[1,2-a]pyridin-6-yl]-2,8-dimethyl-imidazo[1,2-b]pyridazine (60 mg, 164.6 μmol), acetyl acetate (164.65 μmol, 16 μL) and pyridine (164.65 μmol, 13 μL) in DCM (2 mL) was stirred at 25° C. for 16 hours. The mixture was concentrated in vacuo to give crude product which was purified by prep HPLC (Boston Prime C18 150*30 mm*5 um, water (NH₃H₂O+NH₄HCO₃)-ACN as a mobile phase, from 30% to 70%, Gradient Time (min): 10, Flow Rate (ml/min): 25) to give 1-[4-[6-(2,8-dimethylimidazo[1,2-b]pyridazin-6-yl)-8-fluoro-imidazo[1,2-a]pyridin-2-yl]-1-piperidyl]ethanone (25 mg, 37% yield) as a white solid. MS: m/z 407.2 [M+H]⁺; ¹H NMR: (400 MHz, METHANOL-d4) δ ppm=9.00-8.99 (d, J=1.4 Hz, 1H), 7.92 (s, 1H), 7.87-7.76 (m, 2H), 7.58-7.57 (d, J=10.8 Hz, 1H), 4.67-4.60 (m, 1H), 4.09-4.01 (m, 1H), 3.37-3.32 (m, 1H), 3.13-3.04 (m, 1H), 2.89-2.79 (m, 1H), 2.67-2.66 (d, J=0.8 Hz, 3H), 2.48 (s, 3H), 2.22-2.1 (m, 5H), 1.83-1.63 (m, 2H).

### Example 11—Compound 120

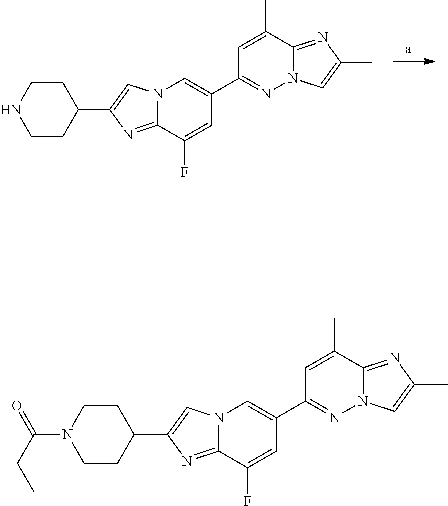 [SMILES: Cc1cn2nc(-c3cc(F)c4nc(C5CCNCC5)cn4c3)cc(C)c2n1 ; CCC(=O)N1CCC(c2cn3cc(-c4cc(C)c5nc(C)cn5n4)cc(F)c3n2)CC1]

6-[8-fluoro-2-(4-piperidyl)imidazo[1,2-a]pyridin-6-yl]-2,8-dimethyl-imidazo[1,2-b]pyridazine (20 mg, 52.14 μmol, 1.0 eq.) was dissolved in DCM (260 μL, 0.2 M) before TEA (15.8 mg, 156.4 μmol, 3.0 eq.) was added. Propanoyl propanoate (7.5 mg, 57.3 μmol, 1.1 eq.) was then slowly added and the solution stirred at rt for 16 hours before being concentrated, taken up in DMSO, filtered, then submitted to HPLC purification (Column: Sunfire C18 100×19 mm, 5 mm; Mobile phase A: MeCN; Mobile phase B: H₂O; Modifier: 0.1% TFA). Obtained 1-[4-[6-(2,8-dimethylimidazo[1,2-b]pyridazin-6-yl)-8-fluoro-imidazo[1,2-a]pyridin-2-yl]-1-piperidyl]propan-1-one (7.2 mg, 26% yield) as a white powder. MS: m/z 421.3 [M+H]⁺; RT: 0.96 min (Method 3) ¹H NMR (600 MHz, DMSO-d6) δ ppm 0.99-1.04 (m, 3H) 1.47-1.55 (m, 1H) 1.60-1.68 (m, 1H) 1.99-2.07 (m, 2H) 2.33-2.39 (m, 2H) 2.44-2.47 (m, 3H) 2.62-2.66 (m, 3H) 2.70-2.77 (m, 1H) 3.01-3.07 (m, 1H) 3.14-3.19 (m, 1H) 3.92-3.97 (m, 1H) 4.44-4.50 (m, 1H) 7.75-7.79 (m, 1H) 7.80-7.85 (m, 1H) 7.95-7.98 (m, 1H) 8.16-8.19 (m, 1H) 9.20-9.22 (m, 1H).

Using the procedure described for Example 11 above, additional compounds described herein were prepared by substituting the appropriate acyl chloride starting material in step a, suitable reagents and reaction conditions, obtaining compounds such as those selected from:

| Cpd ID | Data |
|---|---|
| 121 | Starting material: cyclopropanecarbonyl chloride and 6-(8-fluoro-2- |
| | (piperidin-4-yl)imidazo[1,2-a]pyridin-6-yl)-2,8-dimethylimidazo[1,2- |
| | b]pyridazine |
| | MS: m/z 433.2 [M + H]⁺; RT: 0.37 min (Method 4) |
| | ¹H NMR (600 MHz, DMSO-d6) δ ppm 0.67-0.72 (m, 2 H) 0.72-0.78 (m, |
| | 2 H) 1.50-1.55 (m, 1 H) 1.64-1.71 (m, 1 H) 1.98-2.04 (m, 2 H) 2.09-2.14 |
| | (m, 1 H) 2.43-2.48 (m, 3 H) 2.61-2.66 (m, 3 H) 2.74-2.82 (m, 1 H) 3.03- |
| | 3.09 (m, 1 H) 3.24-3.27 (m, 1 H) 4.32-4.36 (m, 1 H) 4.41-4.49 (m, 1 H) |
| | 7.74-7.78 (m, 1 H) 7.79-7.85 (m, 1 H) 7.96-7.99 (m, 1 H) 8.14-8.20 (m, |
| | 1 H) 9.19-9.23 (m, 1 H) |

### Example 12—Compound 113

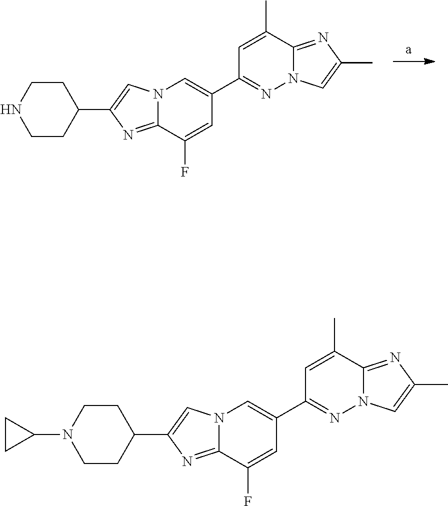 [SMILES: Cc1cn2nc(-c3cc(F)c4nc(C5CCNCC5)cn4c3)cc(C)c2n1 ; Cc1cn2nc(-c3cc(F)c4nc(C5CCN(C6CC6)CC5)cn4c3)cc(C)c2n1]

To a solution of 6-[8-fluoro-2-(4-piperidyl)imidazo[1,2-a]pyridin-6-yl]-2,8-dimethyl-imidazo[1,2-b]pyridazine (50 mg, 137 μmol) in MeOH (1 mL) was added (1-ethoxycyclopropoxy)-trimethyl-silane (36 mg, 206 μmol, 41 μL), TEA (137 μmol, 19 μL) and sodium cyanoborohydride (14 mg, 220 μmol) and the mixture was stirred at 25° C. for 20 min. Then acetic acid (412 μmol, 24 μL) was added and the mixture was stirred at 60° C. for 16 h. The mixture was filtered, and the filtrate was concentrated in vacuo to give the residue, which was purified by prep-HPCL (Column: Boston Green ODS 150×30 mm×5 um; Condition: water (HCl)-ACN; Gradient (% organic): 0-100% optimized for each example; Flow Rate (mL/min) 25) to give 6-[2-(1-cyclopropyl-4-piperidyl)-8-fluoro-imidazo[1,2-a]pyridin-6-yl]-2,8-dimethyl-imidazo[1,2-b]pyridazine (13 mg, 23%) as white solid. MS: m/z 405.1 [M+H]+; RT: 1.31 min.

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 106 | Starting material: 6-(8-fluoro-2-(piperidin-4-yl)imidazo[1,2-a]pyridin-6-yl)- |
| | 2-methylimidazo[1,2-b]pyridazine |
| | MS: m/z 391.3 [M + H]⁺ ]⁺; RT: 1.81 min (Method 1) |
| 167 | Starting material: 8-(difluoromethyl)-6-[8-fluoro-2-(4- |
| | piperidyl)imidazo[1,2-a]pyridin-6-yl]-2-methyl-imidazo[1,2-b]pyridazine |
| | MS: m/z 441.2 [M + H]⁺; RT: 0.49 min (Method 4) |
| | 1H NMR (400 MHz, METHANOL-d4) δ ppm 1.00-1.06 (m, 4 H) 1.97- |
| | 2.09 (m, 2 H) 2.34-2.41 (m, 2 H) 2.53-2.57 (m, 3 H) 2.85-2.92 (m, 1 H) |
| | 3.19-3.25 (m, 1 H) 3.38-3.44 (m, 2 H) 3.77-3.84 (m, 2 H) 7.18-7.47 (m, |
| | 1 H) 7.88-7.94 (m, 1 H) 7.94-7.97 (m, 1 H) 7.98-8.01 (m, 1 H) 8.14-8.16 |
| | (m, 1 H) 9.14-9.18 (m, 1 H) |
| 171 | Starting material: 5-[8-fluoro-2-(4-piperidyl)imidazo[1,2-a]pyridin-6-yl]- |
| | 2,7-dimethyl-pyrazolo[4,3-b]pyridine |
| | MS: m/z 405.3 [M + H]⁺; RT: 0.96 min (Method 3) |
| | 1H NMR (600 MHz, DMSO-d6) δ ppm 0.83-0.90 (m, 2 H) 0.92-1.00 (m, |
| | 2 H) 1.86-1.96 (m, 2 H) 2.23-2.30 (m, 2 H) 2.60-2.65 (m, 3 H) 2.85-2.92 |
| | (m, 1 H) 2.99-3.15 (m, 2 H) 3.61-3.68 (m, 2 H) 4.20-4.26 (m, 3 H) 7.72- |
| | 7.76 (m, 1 H) 7.88-7.92 (m, 1 H) 7.94-8.01 (m, 1 H) 8.59-8.66 (m, 1 H) |
| | 8.79-8.88 (m, 1 H) 9.20-9.26 (m, 1 H). Prod RT 1.80 min, M+405.3 (3.5 |
| | MIN B); Prod RT 0.96 min, M+405.3 (3.5 MIN A) |
| 172 | Starting material: 1-ethyl-7-[8-fluoro-2-(4-piperidyl)imidazo[1,2-a]pyridin- |
| | 6-yl]-3-methyl-pyrrolo[1,2-a]pyrazine |
| | MS: m/z 418.3 [M + H]⁺; RT: 2.13 min (Method 1) |
| | 1H NMR (600 MHz, DMSO-d6) δ ppm 0.84-0.89 (m, 2 H) 0.93-1.00 (m, |
| | 2 H) 1.35-1.43 (m, 3 H) 1.86-1.96 (m, 2 H) 2.24-2.30 (m, 2 H) 2.37-2.43 |
| | (m, 3 H) 3.02-3.12 (m, 4 H) 3.32-3.37 (m, 2 H) 3.65 (br s, 2 H) 7.67-7.76 |
| | (m, 1 H) 7.87-7.93 (m, 1 H) 8.13-8.30 (m, 1 H) 8.36-8.51 (m, 1 H) 8.93- |
| | 9.12 (m, 2 H) |
| 204 | Starting material: 6-[2-[(1S,5R)-3-azabicyclo[3.1.0]hexan-6-yl]-8-fluoro- |
| | imidazo[1,2-a]pyridin-6-yl]-2,8-dimethyl-imidazo[1,2-b]pyridazine |
| | MS: m/z 403.1 [M + H]⁺; RT: 1.44 min (Method 10) |

### Example 13—Compound 129

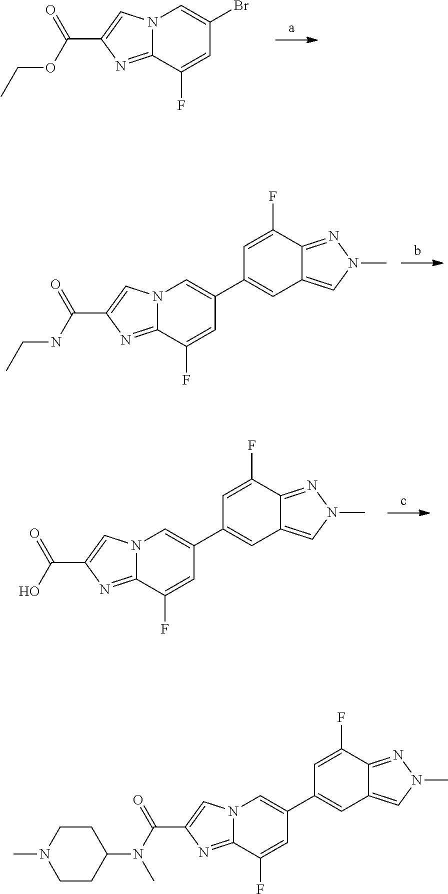

Step a: A mixture of ethyl 6-bromo-8-fluoro-imidazo[1,2-a]pyridine-2-carboxylate (1 g, 3.48 mmol), 7-fluoro-2-methyl-5-(4,4,5,5-tetramethyl-1,3,2-dioxaborolan-2-yl)indazole (1.20 g, 3.83 mmol, hydrochloride), Pd(dppf)Cl₂ DCM (284.46 mg, 348.3 μmol), dicesium carbonate (3.40 g, 10.45 mmol) in dioxane (9.3 mL) and water (2.3 mL) was stirred under N₂ at 90° C. for 4 h. After cooling to rt, the mixture was filtered through celite and MgSO₄ (DCM/EtOAc eluent), concentrated, and purified by silica gel chromatography (24 g, 0-100% 3:1 EtOAc:EtOH in heptane over 10 min) to obtain ethyl 8-fluoro-6-(7-fluoro-2-methyl-indazol-5-yl)imidazo[1,2-a]pyridine-2-carboxylate (250 mg, 20% yield). MS: m/z 357.1 [M+H]⁺

Step b: A mixture of ethyl 8-fluoro-6-(7-fluoro-2-methyl-indazol-5-yl)imidazo[1,2-a]pyridine-2-carboxylate (250 mg, 701.6 μmol) and lithium hydroxide hydrate (58.88 mg, 1.40 mmol) in methanol (467 μL), THF (3.4 mL), water (841 μL) was stirred at rt for 4 h, neutralized with 4 N HCl in dioxane under stirring at rt, concentrated under reduced pressure, and dried under high vacuum to obtain 8-fluoro-6-(7-fluoro-2-methyl-indazol-5-yl)imidazo[1,2-a]pyridine-2-carboxylic acid which was used crude in next reaction (assumed 100% yield). MS: m/z 329.0 [M+H]⁺

Step c: T3P (274.1 μmol, 163 μL, 50% purity in ethyl acetate), followed by NEt₃ (365.55 μmol, 51 μL) were added to a solution of 8-fluoro-6-(7-fluoro-2-methyl-indazol-5-yl)imidazo[1,2-a]pyridine-2-carboxylic acid (30 mg, 91 μmol) and N,1-dimethylpiperidin-4-amine (23.4 mg, 182.7 μmol) in THF (1.1 mL) at 0° C. The ice bath was then removed and the mixture was stirred overnight at rt. The mixture was quenched with water, extracted with EtOAc, dried over MgSO₄, filtered, concentrated, submitted to HPLC purification (Column: Sunfire C18 100×19 mm, 5 mm; Mobile phase A: MeCN; Mobile phase B: H₂O; Modifier: 0.1% TFA) to obtain 8-fluoro-6-(7-fluoro-2-methyl-indazol-5-yl)-N-methyl-N-(1-methyl-4-piperidyl)imidazo[1,2-a]pyridine-2-carboxamide (3.8 mg, 7% yield). MS: m/z 349.2 [M+H]⁺; RT: 0.50 min (Method 4)

Using the procedure described for Example 17 above, additional compounds described herein were prepared by substituting the appropriate boronic ester starting material in step b, suitable reagents and reaction conditions, obtaining compounds such as those selected from:

| Cpd ID | Data |
|---|---|
| 332 | Starting material: tert-butyl 4-(methylamino)piperidine-1-carboxylate, 2,8- |
| | dimethyl-6-(4,4,5,5-tetramethyl-1,3,2-dioxaborolan-2-yl)imidazo[1,2- |
| | b]pyridazine |
| | MS: m/z 436.2 [M + H]⁺; RT: 0.91 min (Method 10) |
| 334 | Starting material: (1-methylpyrrolidin-2-yl)methanamine, 2,8-dimethyl-6- |
| | (4,4,5,5-tetramethyl-1,3,2-dioxaborolan-2-yl)imidazo[1,2-b]pyridazine |
| | MS: m/z 422.3 [M + H]⁺; RT: 1.95 min (Method 8) |
| 331 | Starting material: N1,N1-dimethylcyclobutane-1,3-diamine, 2,8-dimethyl- |
| | 6-(4,4,5,5-tetramethyl-1,3,2-dioxaborolan-2-yl)imidazo[1,2-b]pyridazine |
| | MS: m/z 422.3 [M + H]⁺; RT: 1.75 min (Method 8) |

### Example 14—Compound 355 & 362

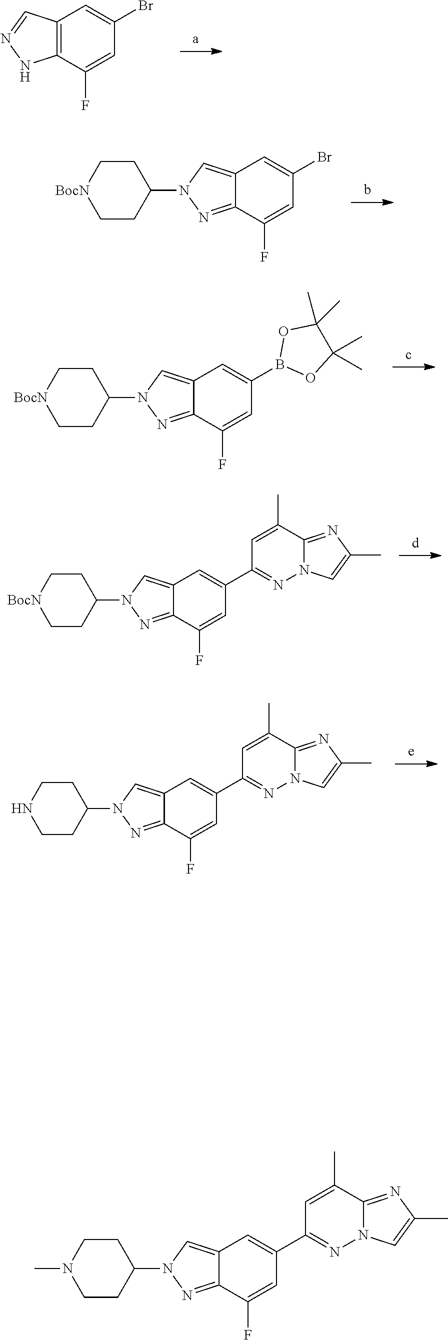

Step a: To a solution of tert-butyl 4-(p-tolylsulfonyloxy)piperidine-1-carboxylate (1.36 g, 3.83 mmol) in DMF (10 mL) was added K₂CO₃ (1.59 g, 11.49 mmol) and 5-bromo-7-fluoro-2H-indazole (823 mg, 3.83 mmol). The mixture was stirred at 100° C. for 12 h. The reaction mixture was then diluted with water (40 mL) and extracted with EtOAc (10 mL×3). The combined organic layers were washed with brine, dried over Na₂SO₄, filtered and concentrated under reduced pressure to give the crude residue. The crude was purified by flash silica gel chromatography (from PE/EtOAc=10/1 to 5/1) to yield the product. Compound tert-butyl 4-(5-bromo-7-fluoro-indazol-2-yl)piperidine-1-carboxylate (492 mg, 1.24 mmol, 32.26% yield) was obtained as yellow oil. MS: m/z 342.0 [M+H]⁺; RT: 0.52 min (Method 3)

Step b: To a mixture of tert-butyl 4-(5-bromo-7-fluoro-2H-indazol-2-yl)piperidine-1-carboxylate (5 g, 12.6 mmol) in Dioxane (120 mL) was added Bis(pinacolato)diboron (3.83 g, 15.1 mmol), KOAc (2.46 g, 25.1 mmol) and Pd(dppf)Cl2(459.3 mg, 627.7 umol) at 25° C. The mixture was stirred at 120° C. for 16 hours under a nitrogen atmosphere. The mixture was concentrated and then water (150 mL) was added. The mixture was extracted with EtOAc (50 mL×3). The combined organic layers were washed with brine (50 mL), dried over Na₂SO₄, filtered and concentrated. The crude material was then purified by flash chromatography (PE/EtOAc=5/1 to 2/1), to yield tert-butyl 4-(7-fluoro-5-(4,4,5,5-tetramethyl-1,3,2-dioxaborolan-2-yl)-2H-indazol-2-yl)piperidine-1-carboxylate (4.85 g, 10.9 mmol, 87% yield) as a white solid. MS: m/z 446.3 [M+H]⁺; RT: 0.748 min (Method 3)

Step c: To a solution of tert-butyl 4-[7-fluoro-5-(4,4,5,5-tetramethyl-1,3,2-dioxaborolan-2-yl)indazol-2-yl]piperidine-1-carboxylate (50 mg, 112 umol) in Dioxane/H₂O (5 mL) was added 5-chloro-2,7-dimethyl-pyrazolo[1,5-a]pyrimidine (20 mg, 112.28 umol), K₂CO₃ (46 mg, 336.8 umol) and PdCl₂(dppf) (8 mg, 11.23 umol). The reaction mixture was stirred at 90° C. for 2 h. The mixture was filtered and concentrated to give a residue. The residue was purified by flash silica gel chromatography (from PE/EtOAc=5/1 to 1/3), to yield the product. tert-butyl 4-[5-(2,7-dimethylpyrazolo[1,5-a]pyrimidin-5-yl)-7-fluoro-indazol-2-yl]piperidine-1-carboxylate (30 mg, 59.22 umol) was obtained as a yellow solid. MS: m/z 465.3 [M+H]⁺; RT: 0.468 min (Method 3)

Step d: tert-butyl 4-[5-(2,8-dimethylimidazo[1,2-b]pyridazin-6-yl)-7-fluoro-indazol-2-yl]piperidine-1-carboxylate (78 mg, 168 umol) was dissolved into MeOH (4 mL) and treated with HCl 4N in dioxane (292 uL) and stirred at room temp for 24 hours. The reaction was concentrated and used crude in the next step. MS: m/z 365.1 [M+H]⁺; RT: 0.45 min (Method 4)

Step e: 6-(7-fluoro-2-(piperidin-4-yl)-2H-indazol-5-yl)-2,8-dimethylimidazo[1,2-b]pyridazine (40 mg, 109.8 umol) was dissolved in THF (1 mL) with paraformaldehyde (131 mg, 109 umol, 149 uL), Acetic acid (66 mg, 1.1 mmol, 63 uL), Sodium cyanoborohydride (21 mg, 329 umol) and stirred at 70+C overnight. The resulting was diluted with water, extracted with EtOAc and dried over sodium sulfate to be purified by FCC using a gradient of 0-100% EtOAc-Heptane to afford the title compound (41.5 mg, 36 umol). MS: m/z 379.1 [M+H]⁺; RT: 1.63 m z (Method 3)

Using the procedure described for Example 17 above, additional compounds described herein were prepared by substituting the appropriate boronic ester starting material in step b, suitable reagents and reaction conditions, obtaining compounds such as those selected from:

| Cpd ID | Data |
|---|---|
| 136 | Starting material: 8-fluoro-2-methyl-6-(4,4,5,5-tetramethyl-1,3,2- |
| | dioxaborolan-2-yl)imidazo[1,2-a]pyridine |
| | MS: m/z 368.1 [M + H]⁺; RT: 0.87 min (Method 3) |
| 351 | Starting material: 5-bromo-2,7-dimethyl-indazole |
| | MS: m/z 364.3 [M + H]⁺; RT: 0.53 min (Method 4) |
| 352 | Starting material: 6-chloro-2-(difluoromethyl)imidazo[1,2-b]pyridazine |
| | MS: m/z 386.9 [M + H]⁺; RT: 0.63 min (Method 4) |
| 353 | Starting material: 5-chloro-2,7-dimethyl-pyrazolo[4,3-b]pyridine |
| | MS: m/z 364.9 [M + H]⁺; RT: 0.51 min (Method 4) |
| 356 | Starting material: 5-bromo-2,7-dimethyl-indazole |
| | MS: m/z 378.3 [M + H]⁺; RT: 1.28 min (Method 3) |
| 360 | Starting material: 5-chloro-2,7-dimethyl-pyrazolo[1,5-a]pyrimidine |
| | MS: m/z 379.2 [M + H]⁺; RT: 0.648 min (Method 4) |
| 366 | Starting material: tert-butyl 3-(tosyloxy)pyrrolidine-1-carboxylate |
| | MS: m/z 365 [M + H]⁺; RT: 0.79 min (Method 4) |
| 368 | Starting material: [4-[tert-butoxycarbonyl(methyl)amino]cyclohexyl] |
| | methanesulfonate |
| | MS: m/z 393.2 [M + H]⁺; RT: 0.9 min (Method 3) |
| 369 | Starting material: 1,3-dimethyl-7-(4,4,5,5-tetramethyl-1,3,2-dioxaborolan- |
| | 2-yl)pyrrolo[1,2-a]pyrazine |
| | MS: m/z 364.2 [M + H]⁺; RT: 0.82 min (Method 3) |
| 364 | Starting material: 6-chloro-2-methyl-imidazo[1,2-b]pyridazine |
| | MS: m/z 365.0 [M + H]⁺; RT: 0.37 min (Method 4) |

### Example 15—Compound 135

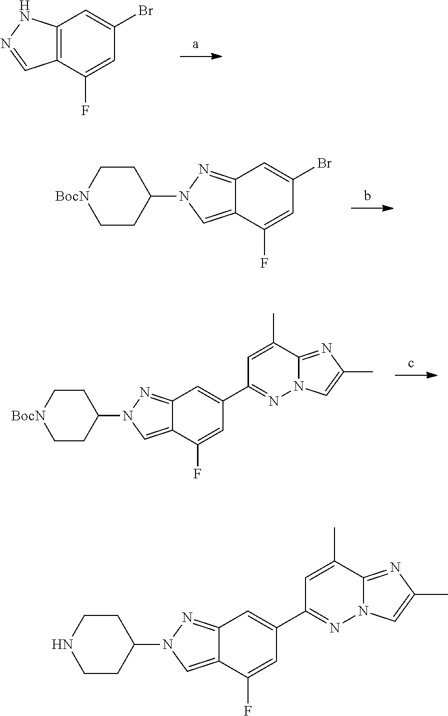 [SMILES: Fc1cc(Br)cc2[nH]ncc12 ; CC(C)(C)OC(=O)N1CCC(n2cc3c(F)cc(Br)cc3n2)CC1 ; Cc1cn2nc(-c3cc(F)c4cn(C5CCN(C(=O)OC(C)(C)C)CC5)nc4c3)cc(C)c2n1 ; Cc1cn2nc(-c3cc(F)c4cn(C5CCNCC5)nc4c3)cc(C)c2n1]

Step a: 6-bromo-4-fluoro-2H-indazole (1 g, 4.65 mmol) was dissolved in DMF (10 mL). Potassium carbonate (6.4 g, 46.5 mmol), tert-butyl 4-iodopiperidine-1-carboxylate (1.45 g, 4.65 mmol) were added and heated to 100° C. overnight. The resulting was diluted with water and EtOAc, extracted with EtOAc and the crude purified by silica column chromatography using a gradient of 0-100% EtOAc-heptane to afford tert-butyl 4-(6-bromo-4-fluoro-2H-indazol-2-yl)piperidine-1-carboxylate (60 mg, 3% yield) MS: m/z 341.8 [M-tBu+H]⁺

Step b: A mixture of tert-butyl 4-(6-bromo-4-fluoro-indazol-2-yl)piperidine-1-carboxylate (30 mg, 75 umol), 2,8-dimethyl-6-(4,4,5,5-tetramethyl-1,3,2-dioxaborolan-2-yl)imidazo[1,2-b]pyridazine (21 mg, 75 μmol), PdCl2(dippf) (45 mg, 7.5 μmol) and Cs₂CO₃ (49 mg, 151 μmol) in dioxane (3 mL) and water (847 μL) was degassed with N₂ and then heated in the microwave for 1 h at 100° C. The resulting was diluted with water and EtOAc and the phases separated and concentrated to afford tert-butyl 4-(6-(2,8-dimethylimidazo[1,2-b]pyridazin-6-yl)-4-fluoro-2H-indazol-2-yl)piperidine-1-carboxylate which was used crude (assumed 100% yield). MS: m/z 465.1 [M+H]⁺

Step c: Tert-butyl 4-(6-(2,8-dimethylimidazo[1,2-b]pyridazin-6-yl)-4-fluoro-2H-indazol-2-yl)piperidine-1-carboxylate (30 mg, 75 μmol) was dissolved in DCM (1 mL) and treated with TFA (0.5 mL) and stirred at room temperature for 2 hours. The reaction was concentrated and purified by reverse phase HPLC using Waters XSelect CSH Prep C18 5 um OBD 30×100 mm and a 5-65% gradient of ACN-water using a TFA modifier (2.8 mg, 10% yield). MS: m/z 365.1 [M+H]⁺; RT 1.01 min (Method 3)

### Example 16—Compound 139 and/or 376

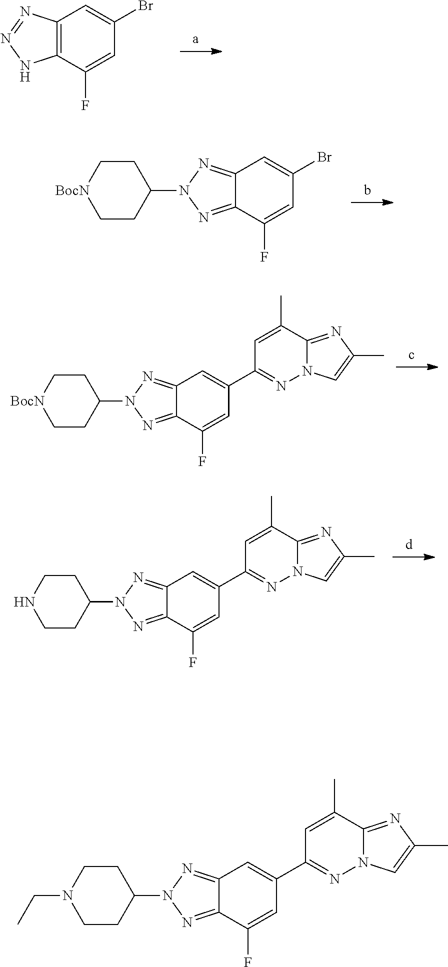

Step a: 6-Bromo-4-fluoro-2H-benzotriazole (500 mg, 2.31 mmol, 1.0 eq.) was dissolved in tetrahydrofuran (11.57 mL, 0.2 M) before tert-butyl 4-hydroxypiperidine-1-carboxylate (465.86, 2.31 mmol, 1.0 eq.) and triphenylphosphine (607.11 mg, 2.31 mmol, 1.0 eq.) were added. DIAD (468 mg, 456 μL, 2.31 mmol, 1.0 eq) was then slowly added and the solution was stirred at rt for 16 hours before being concentrated and injected directly onto normal phase silica column for purification using 0-100% EtOAc:heptane over 7 minutes. Product elutes at 70% EtOAc. Identified fractions were collected, concentrated, to yield tert-butyl 4-(6-bromo-4-fluoro-benzotriazol-2-yl)piperidine-1-carboxylate (488.5 mg, 1.14 mmol, 49% yield) which was carried forward as is. MS: m/z 344.9 [M+H]⁺; RT 1.05 min (Method 4).

Step b: Tert-butyl 4-(6-bromo-4-fluoro-benzotriazol-2-yl)piperidine-1-carboxylate (100 mg, 232.93 μmol, 1.0 eq.) was dissolved in water (376 μL, 0.186 M) and dioxane (877 μL, 0.186 M) before 2,8-dimethyl-6-(4,4,5,5-tetramethyl-1,3,2-dioxaborolan-2-yl)imidazo[1,2-b]pyridazine (76.35 mg, 279.52 μmol, 1.2 eq) and cesium carbonate (303.6 mg, 931.7 μmol, 4.0 eq.) were added. The solution was then degassed with nitrogen before Pd(dppf)₂Cl₂ DCM adduct (15 mg, 18.4 μmol, 0.1 eq) was added and the solution was heated to 90° C. for 6 hours before being concentrated and dry loaded onto normal phase and purified via 0-25% MeOH:DCM over 12 minutes. Product eluted at 10% MeOH. Identified fractions were collected, combined, and concentrated then carried forward as is (111.3 mg, 231.9 μmol, 99% yield). MS: m/z 466.2 [M+H]⁺; RT 0.74 min (Method 4).

Step c: Tert-butyl 4-[6-(2,8-dimethylimidazo[1,2-b]pyridazin-6-yl)-4-fluoro-benzotriazol-2-yl]piperidine-1-carboxylate (111.3 mg, 231.9 μmol, 1.0 eq.) was dissolved in DCM (1.16 mL, 0.2 M) before 4 M HCl in dioxane (579.7 μL, 2.32 mmol, 10 eq.) was added and the solution was heated to 40° C. for 1 hour. A precipitate had formed that was further precipitated with addition of a few mL of diethyl ether. The product was filtered off, washed with diethyl ether and dried to obtain 6-(2,8-dimethylimidazo[1,2-b]pyridazin-6-yl)-4-fluoro-2-(piperidin-4-yl)-2H-benzo[d][1,2,3]triazole (87.1 mg, 216.7 μmol, 93% yield) as a white powder. MS: m/z 366.2 [M+H]⁺; RT: 0.44 min (Method 4). ¹H NMR (400 MHz, METHANOL-d4) δ ppm 2.54-2.66 (m, 4H) 2.66-2.67 (m, 3H) 2.79-2.83 (m, 3H) 3.34-3.42 (m, 2H) 3.60-3.67 (m, 2H) 5.32-5.40 (m, 1H) 7.98-8.04 (m, 1H) 8.34-8.40 (m, 2H) 8.59-8.62 (m, 1H)

Step d: 6-(2,8-dimethylimidazo[1,2-b]pyridazin-6-yl)-4-fluoro-2-(4-piperidyl)benzotriazole (10 mg, 27.37 umol, 1.0 eq.) was dissolved in Acetonitrile (136.83 uL, 0.2 M) before acetaldehyde (3.62 mg, 82.10 umol, 3.0 eq.) and Acetic acid (8.22 mg, 136.83 umol, 5.0 eq.) were added. The solution then stirred for 5 minutes before Sodium cyanoborohydride (3.44 mg, 54.73 umol, 2.0 eq.) was added. The solution then stirred for 15 minutes before it was concentrated then taken back up in DMSO, water, and methanol then purified via HPLC purification (Column: Sunfire C18 100×19 mm, 5 mm; Mobile phase A: MeCN; Mobile phase B: H2O; Modifier: 0.1% TFA). Identified fractions were collected, combined, and concentrated to yield 6-(2,8-dimethylimidazo[1,2-b]pyridazin-6-yl)-2-(1-ethyl-4-piperidyl)-4-fluoro-benzotriazole (3.9 mg, 28.08% yield, 100% purity) as a white powder that was registered as is. MS: m/z 394.1 [M+H]⁺; RT: 2.33 min (Method 1). ¹H NMR (600 MHz, DMSO-d6) δ ppm 1.25-1.30 (m, 3H) 2.41-2.43 (m, 3H) 2.43-2.49 (m, 2H) 2.62-2.65 (m, 3H) 3.08-3.18 (m, 2H) 3.19-3.29 (m, 4H) 3.70-3.76 (m, 2H) 5.26-5.35 (m, 1H) 7.82-7.93 (m, 1H) 7.96-8.04 (m, 1H) 8.06-8.13 (m, 1H) 8.56-8.60 (m, 1H) 9.43-9.55 (m, 1H)

Using the procedure described for Example 17 above, additional compounds described herein were prepared by substituting the appropriate boronic ester starting material in step b, suitable reagents and reaction conditions, obtaining compounds such as those selected from:

| Cpd ID | Data |
|---|---|
| 137 | Starting material: 8-fluoro-2-methyl-6-(4,4,5,5-tetramethyl-1,3,2- |
| | dioxaborolan-2-yl)imidazo[1,2-a]pyridine |
| | MS: m/z 369.1 [M + H]⁺; RT: 0.84 min (Method 4) |
| 138 | Starting material: 7-fluoro-2-methyl-5-(4,4,5,5-tetramethyl-1,3,2- |
| | dioxaborolan-2-yl)-2H-indazole |
| | MS: m/z 369.0 [M + H]⁺; RT: 1.32 min (Method 3) |
| | ¹H NMR (400 MHz, CHLOROFORM-d) δ ppm 2.57-2.66 (m, 4 H) 3.36- |
| | 3.41 (m, 2 H) 3.60-3.65 (m, 2 H) 4.25-4.28 (m, 3 H) 5.27-5.32 (m, 1 H) |
| | 7.40-7.45 (m, 1 H) 7.53-7.57 (m, 1 H) 7.86-7.88 (m, 1 H) 7.95-7.97 (m, |
| | 1 H) 8.38-8.40 (m, 1 H) |
| 378 | Starting material: 1,3-dimethyl-7-(4,4,5,5-tetramethyl-1,3,2-dioxaborolan- |
| | 2-yl)pyrrolo[1,2-a]pyrazine |
| | MS: m/z 365.2 [M + H]⁺; RT: 0.96 min (Method 3) |
| | 1H NMR (400 MHz, MeOD-d) δ ppm 2.50-2.53 (m, 3 H) 2.58-2.65 (m, |
| | 4 H) 2.97-3.00 (m, 3 H) 3.36-3.42 (m, 2 H) 3.58-3.67 (m, 3 H) 5.28- |
| | 5.34 (m, 1 H) 7.69-7.74 (m, 1 H) 8.17-8.20 (m, 1 H) 8.23-8.25 (m, 1 H) |
| | 8.30-8.32 (m, 1 H) 8.64-8.67 (m, 1 H) |
| 379 | Starting material: 1-ethyl-3-methyl-7-(4,4,5,5-tetramethyl-1,3,2- |
| | dioxaborolan-2-yl)pyrrolo[1,2-a]pyrazine |
| | MS: m/z 379.2 [M + H]⁺; RT: 0.49 min (Method 4) |
| | 1H NMR (400 MHz, CHLOROFORM-d) δ ppm 1.50-1.59 (m, 3 H) 2.49 |
| | (br d, J = 1.00 Hz, 3 H) 2.55-2.70 (m, 5 H) 3.35-3.42 (m, 2 H) 3.58- |
| | 3.68 (m, 3 H) 5.28-5.36 (m, 1 H) 7.68-7.76 (m, 1 H) 8.14-8.21 (m, 1 H) |
| | 8.21-8.25 (m, 1 H) 8.25-8.31 (m, 1 H) 8.59-8.66 (m, 1 H) |
| 382 | Starting material: 7-(difluoromethyl)-2-methyl-5-(4,4,5,5-tetramethyl- |
| | 1,3,2-dioxaborolan-2-yl)indazole |
| | MS: m/z 401.3 [M + H]⁺; RT: 1.43 min (Method 3) |
| 383 | Starting material: tert-butyl 3-(hydroxymethyl)azetidine-1-carboxylate |
| | MS: m/z 352.1 [M + H]⁺; RT: 1.62 min (Method 10) |
| 384 | Starting material: 2-methyl-6-(4,4,5,5-tetramethyl-1,3,2-dioxaborolan-2- |
| | yl)imidazo[1,2-b]pyridazine |
| | MS: m/z 352.1 [M + H]⁺; RT: 1.00 min (Method 3) |
| | 1H NMR (600 MHz, DMSO-d6) δ ppm 2.35-2.41 (m, 2 H) 2.42-2.47 (m, |
| | 3 H) 2.49-2.55 (m, 4 H) 3.19-3.27 (m, 2 H) 5.30-5.37 (m, 1 H) 7.99- |
| | 8.03 (m, 1 H) 8.16-8.19 (m, 1 H) 8.51-8.60 (m, 1 H) 8.60-8.63 (m, 1 H) |
| | 8.75-8.84 (m, 1 H) |
| 385 | Starting material: tert-butyl 6-hydroxy-2-azaspiro[3.3]heptane-2- |
| | carboxylate |
| | MS: m/z 378.1 [M + H]⁺; RT: 1.33 min (Method 10) |
| 386 | Starting material: tert-butyl (3S)-3-(hydroxymethyl)pyrrolidine-1- |
| | carboxylate |
| | MS: m/z 366.1 [M + H]⁺; RT: 1.33 min (Method 10) |
| 393 | Starting material: tert-butyl rel-(2R,6R)-4-hydroxy-2,6-dimethyl- |
| | piperidine-1-carboxylate |
| | MS: m/z 394.3 [M + H]⁺; RT: 2.25 min (Method 1) |
| | 1H NMR (600 MHz, DMSO-d6) δ ppm 1.30-1.32 (m, 3 H) 1.48-1.50 (m, |
| | 3 H) 2.10-2.17 (m, 1 H) 2.38-2.41 (m, 1 H) 2.44-2.46 (m, 3 H) 2.46- |
| | 2.48 (m, 1 H) 2.51-2.53 (m, 1 H) 2.63-2.66 (m, 3 H) 3.73-3.75 (m, 1 H) |
| | 4.01-4.04 (m, 1 H) 5.50-5.55 (m, 1 H) 7.96-8.02 (m, 2 H) 8.15-8.19 (m, |
| | 1 H) 8.57-8.59 (m, 1 H) |
| 394 | Starting material: tert-butyl rac-4-hydroxy-2,2-dimethyl-piperidine-1- |
| | carboxylate |
| | MS: m/z 394.3 [M + H]⁺; RT: 1.34 min (Method 3) |
| | 1H NMR (600 MHz, DMSO-d6) δ ppm 1.13 (s, 3 H) 1.18-1.20 (m, 3 H) 1.83- |
| | 1.89 (m, 1 H) 1.93-1.99 (m, 1 H) 2.09-2.13 (m, 1 H) 2.17-2.21 (m, 1 H) 2.40- |
| | 2.42 (m, 3 H) 2.61-2.62 (m, 3 H) 2.92-2.96 (m, 2 H) 5.15-5.22 (m, 1 H) 7.83- |
| | 7.85 (m, 1 H) 7.91-7.95 (m, 1 H) 8.08-8.09 (m, 1 H) 8.50-8.52 (m, 1 H). |
| 395 | Starting material: tert-butyl 2-hydroxy-7-azaspiro[3.5]nonane-7- |
| | carboxylate |
| | MS: m/z 406.3 [M + H]⁺; RT: 1.36 min (Method 3) |
| | 1H NMR (600 MHz, DMSO-d6) δ ppm 1.85-1.88 (m, 2 H) 1.92-1.95 (m, 2 H) |
| | 2.40-2.47 (m, 3 H) 2.57-2.62 (m, 2 H) 2.63-2.66 (m, 3 H) 2.68-2.73 (m, 2 H) |
| | 3.01-3.07 (m, 2 H) 3.12 (br s, 2 H) 5.64 (quin, J = 8.20 Hz, 1 H) 7.94-8.02 (m, 2 |
| | H) 8.14-8.19 (m, 1 H) 8.55-8.58 (m, 1 H). |
| 396 | Starting material: tert-butyl rel-(5S,8S)-8-hydroxy-2-azaspiro[4.4]nonane- |
| | 2-carboxylate |
| | MS: m/z 406.3 [M + H]⁺; RT: 1.40 min (Method 3) |
| | 1H NMR (600 MHz, DMSO-d6) δ ppm 1.80-1.85 (m, 1 H) 2.07 (s, 4 H) 2.33- |
| | 2.42 (m, 2 H) 2.43-2.45 (m, 3 H) 2.62-2.66 (m, 3 H) 3.13-3.33 (m, 6 H) 5.56- |
| | 5.65 (m, 1 H) 7.94-8.00 (m, 2 H) 8.14-8.18 (m, 1 H) 8.54-8.58 (m, 1 H) |
| 397 | Starting material: tert-butyl rac-(1R,5S,6S)-6-hydroxy-3- |
| | azabicyclo[3.2.0]heptane-3-carboxylate |
| | MS: m/z 378.3 [M + H]⁺; RT: 1.29 min (Method 3) |
| | 1H NMR (600 MHz, DMSO-d6) δ ppm 2.44 (s, 3 H) 2.57-2.62 (m, 1 H) 2.63- |
| | 2.65 (m, 3 H) 2.93-2.98 (m, 1 H) 3.20-3.29 (m, 3 H) 3.35-3.41 (m, 1 H) 3.52 |
| | (br dd, J = 11.83, 4.20 Hz, 1 H) 3.74-3.77 (m, 1 H) 3.80-3.83 (m, 1 H) 5.45-5.50 |
| | (m, 1 H) 7.93-7.97 (m, 1 H) 7.97-8.01 (m, 1 H) 8.15-8.17 (m, 1 H) 8.57-8.59 |
| | (m, 1 H). Prod RT 1.29 min, M+378.3 (A). Prod RT 2.08 min M+378.3 (B). |
| 398 | Starting material: tert-butyl 2-hydroxy-6-azaspiro[3.4]octane-6-carboxylate |
| | MS: m/z 392.3 [M + H]⁺; RT: 1.18 min (Method 3) |
| | 1H NMR (600 MHz, DMSO-d6) δ ppm 2.10-2.14 (m, 2 H) 2.41-2.44 (m, 3 H) |
| | 2.60-2.64 (m, 3 H) 2.82-2.89 (m, 4 H) 3.19-3.24 (m, 2 H) 3.39-3.42 (m, 2 H) |
| | 5.63-5.70 (m, 1 H) 7.87-7.90 (m, 1 H) 7.94-7.98 (m, 1 H) 8.08-8.13 (m, 1 H) |
| | 8.52-8.55 (m, 1 H) |
| 402 | Starting material: tert-butyl 4-hydroxy-2,2-dimethyl-pyrrolidine-1- |
| | carboxylate |
| | MS: m/z 380.2 [M + H]⁺; RT: 1.10 min (Method 3) |
| 401 | Starting material: tert-butyl 4-hydroxy-2-methyl-piperidine-1-carboxylate |
| | MS: m/z 380.2 [M + H]⁺; RT: 1.09 min (Method 3) |
| 400 | Starting material: tert-butyl 7-hydroxy-4-azaspiro[2.5]octane-4-carboxylate |
| | MS: m/z 392.2 [M + H]⁺; RT: 1.11 min (Method 3) |
| 403 | Starting material: tert-butyl (6S)-6-hydroxy-2-azaspiro[3.4]octane-2- |
| | carboxylate |
| | MS: m/z 392.2 [M + H]⁺; RT: 1.05 min (Method 3) |
| 404 | Starting material: tert-butyl-3-hydroxypyrrolidin-1-ylazetidine-1- |
| | carboxylate |
| | MS: m/z 407.2 [M + H]⁺; RT: 0.93 min (Method 3) |
| 405 | Starting material: tert-butyl (3a,6a)-5-hydroxy-3,3a,4,5,6,6a-hexahydro- |
| | 1H-cyclopenta[c]pyrrole-2-carboxylate |
| | MS: m/z 392.2 [M + H]⁺; RT: 1.05 min (Method 3) |
| 406 | Starting material: tert-butyl-4-hydroxy-1-oxa-9-azaspiro[5.5]undecane-9- |
| | carboxylate |
| | MS: m/z 436.2 [M + H]⁺; RT: 1.12 min (Method 3) |
| 407 | Starting material: 3-(dimethylamino)cyclopentanol |
| | MS: m/z 394.2 [M + H]⁺; RT: 1.03 min (Method 3) |
| 410 | Starting material: tert-butyl (6R)-6-hydroxy-2-azaspiro[3.4]octane-2- |
| | carboxylate |
| | MS: m/z 392.2 [M + H]⁺; RT: 1.08 min (Method 3) |
| 558 | Starting material: tert-butyl (7S)-7-hydroxy-5-oxa-2-azaspiro[3.4]octane-2- |
| | carboxylate |
| | MS: m/z 394.2 [M + H]⁺; RT: 0.85 min (Method 3) |
| 562 | Starting material: tert-butyl-6-hydroxy-2-azaspiro[3.5]nonane-2- |
| | carboxylate |
| | MS: m/z 406.2 [M + H]+; RT: 1.14 min (Method 3) |
| 564 | Starting material: tert-butyl (7R)-7-hydroxy-5-oxa-2-azaspiro[3.4]octane-2- |
| | carboxylate |
| | MS: m/z 394.2 [M + H]⁺; RT: 0.99 min (Method 3) |
| 565 | SM: tert-butyl rac-5-hydroxy-1,3,3a,4,5,6,7,7a-octahydroisoindole-2- |
| | carboxylate |
| | MS: m/z 406.3 [M + H]⁺; RT: 0.50 min (Method 4). |

### Example 17—Compound 140

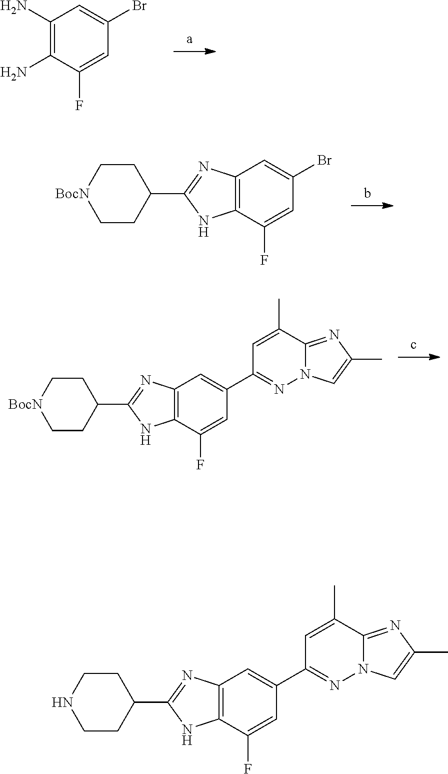

Step a: To a solution of 1-(tert-butoxycarbonyl) piperidine-4-carboxylic acid (10 g, 43.62 mmol) in DMF (75 mL) and pyridine (75 mL) was added CDI (7.78 g, 47.9 mmol). The mixture was stirred at 60° C. for 4 h. 5-bromo-3-fluorobenzene-1,2-diamine (8.94 g, 43.62 mmol) was added and the whole mixture was stirred at 30° C. for 12 h. The mixture was concentrated under reduced pressure. Then AcOH (350 mL) was added and the whole mixture was stirred at 100° C. for 1 h. The mixture was concentrated and then sat. aq. NaHCO₃ solution (200 mL) was added. The mixture was extracted with EtOAc (200 mL×3). The combined organic layers were washed with brine (300 mL), dried over Na₂SO₄, filtered and concentrated. The mixture was filtered, and the filtrate was purified by preparative HPLC (Column YMC-Triart Prep C18 150*40 mm*7 um; Condition water (NH₃H₂O+NH₄HCO₃)-CAN, Begin B 36, End B 76, Gradient Time (min) 9; FlowRate (ml/min): 60) followed by lyophilization to yield tert-butyl 4-(5-bromo-7-fluoro-1H-benzo[d]imidazol-2-yl)piperidine-1-carboxylate (3.12 g, 17% yield) as yellow solid. MS: m/z 400.1 [M+H]⁺.

Step b: A mixture of tert-butyl 4-(5-bromo-7-fluoro-1H-benzo[d]imidazol-2-yl)piperidine-1-carboxylate (39 mg, 0.10 mmol), 2,8-dimethyl-6-(4,4,5,5-tetramethyl-1,3,2-dioxaborolan-2-yl)imidazo[1,2-b]pyridazine (41 mg, 0.15 mmol), XPhos Pd G3 (8.5 mg, 0.01 mmol) and K₃PO₄ (64 mg, 0.3 mmol) in dioxane (0.8 mL) and water (0.2 mL) was stirred at 60° C. for 2 hours. The reaction solution was washed with brine and extracted with EtOAc (2 mL, 2 times.) The combined organics were dried over MgSO₄ and concentrated in vacuo to afford tert-butyl 4-(5-(2,8-dimethylimidazo[1,2-b]pyridazin-6-yl)-7-fluoro-1H-benzo[d]imidazol-2-yl)piperidine-1-carboxylate which was taken forward to the next step without further purification (assumed 100% yield). MS: m/z 465.2 [M+H]⁺.

Step c: To a mixture of tert-butyl 4-(5-(2,8-dimethylimidazo[1,2-b]pyridazin-6-yl)-7-fluoro-1H-benzo[d]imidazol-2-yl)piperidine-1-carboxylate (46.4 mg, 0.1 mmol) in hexafluoro isopropanol (1 mL) was added TFA (23 μL). After stirring for 16 hours at room temperature, the reaction mixture was diluted with 10 mL DCM and transferred to a separatory funnel with 25 mL sat. aq. NaHCO₃. The layers were separated, and the aq. extracted 2× further with DCM. The combined organics were dried over sodium sulfate and concentrated in vacuo to provide the crude product which was purified via preparative HPLC (Waters XSelect CSH Prep C18 5 um OBD 30×100 mm and a 5-65% gradient of ACN-water, 0.1% TFA modifier) to afford 6-(7-fluoro-2-(piperidin-4-yl)-1H-benzo[d]imidazol-5-yl)-2,8-dimethylimidazo[1,2-b]pyridazine (4 mg, 11%). MS: m/z 365.2 [M+H]⁺. ¹H NMR (400 MHz, METHANOL-d₄) δ ppm 1.83-1.98 (m, 2H), 2.10 (br d, J=12.01 Hz, 2H), 2.48 (s, 3H), 2.66 (s, 3H), 2.80 (br t, J=11.76 Hz, 2H), 3.06-3.25 (m, 3H), 7.58 (s, 1H), 7.67 (d, J=12.01 Hz, 1H), 7.89 (s, 1H), 7.96 (s, 1H)

Using the procedure described for Example 17 above, additional compounds described herein were prepared by substituting the appropriate boronic ester or acid equivalent in step b, suitable reagents and reaction conditions, obtaining compounds such as those selected from:

| Cpd ID | Data |
|---|---|
| 141 | Starting material: 7-fluoro-2-methyl-5-(4,4,5,5-tetramethyl-1,3,2- |
| | dioxaborolan-2-yl)-2H-indazole |
| | MS: m/z 368.1 [M + H]⁺; RT: 1.03 (Method 3) |
| 142 | Starting material: 2,7-dimethyl-5-(4,4,5,5-tetramethyl-1,3,2-dioxaborolan-2- |
| | yl)-2H-indazole |
| | MS: m/z 364.2 [M + H]⁺; RT: 1.03 (Method 3) |
| 143 | Starting material: 8-fluoro-2-methyl-6-(4,4,5,5-tetramethyl-1,3,2- |
| | dioxaborolan-2-yl)imidazo[1,2-a]pyridine |
| | MS: m/z 368.2 [M + H]⁺; RT: 0.73 (Method 3) |
| 144 | Starting material: 8-chloro-2-methyl-6-(4,4,5,5-tetramethyl-1,3,2- |
| | dioxaborolan-2-yl)imidazo[1,2-a]pyridine |
| | MS: m/z 384.1 [M + H]⁺; RT: 0.78 (Method 3) |
| 145 | Starting material: 1,3-dimethyl-7-(4,4,5,5-tetramethyl-1,3,2-dioxaborolan-2- |
| | yl)pyrrolo[1,2-a]pyrazine |
| | MS: m/z 364.1 [M + H]⁺; RT: 0.78 (Method 3) |
| | ¹H NMR (400 MHz, METHANOL-d₄) δ ppm 2.14-2.28 (m, 2 H), 2.35- |
| | 2.47 (m, 2 H), 2.52 (s, 3 H), 2.98 (s, 3 H), 3.25 (td, J = 12.76, 2.75 Hz, 2 H), |
| | 3.38-3.47 (m, 1 H), 3.54-3.64 (m, 2 H), 7.57 (dd, J = 11.76, 1.00 Hz, 1 H), |
| | 7.87 (d, J = 1.00 Hz, 1 H), 8.07 (s, 1 H), 8.28 (s, 1 H), 8.58 (d, J = 1.00 Hz, 1 |
| | H) |
| 146 | Starting material: 2-methyl-5-(4,4,5,5-tetramethyl-1,3,2-dioxaborolan-2-yl)- |
| | 2H-benzo[d][1,2,3]triazole |
| | MS: m/z 351.2 [M + H]⁺; RT: 0.47 min (Method 4) |
| 147 | Starting material: 2,8-dimethyl-6-(4,4,5,5-tetramethyl-1,3,2-dioxaborolan-2- |
| | yl)imidazo[1,2-a]pyridine |
| | MS: m/z 364.0 [M + H]⁺; RT: 0.37 min (Method 4) |
| 148 | Starting material: 4-fluoro-2-methyl-6-(4,4,5,5-tetramethyl-1,3,2- |
| | dioxaborolan-2-yl)-2H-benzo[d][1,2,3]triazole |
| | MS: m/z 369.2 [M + H]⁺; RT: 0.52 min (Method 4) |
| 149 | Starting material: 8-fluoro-2-methyl-6-(4,4,5,5-tetramethyl-1,3,2- |
| | dioxaborolan-2-yl)-[1,2,4]triazolo[1,5-a]pyridine |
| | MS: m/z 369.2 [M + H]⁺; RT: 0.40 min (Method 4) |
| 417 | Starting material: 1-(2-methoxyethyl)piperidine-4-carbaldehyde |
| | MS: m/z 423.2 [M + H]⁺; RT: 0.86 min (Method 3) |
| 418 | Starting material: 2-methylpiperidine-4-carbaldehyde |
| | MS: m/z 379.1 [M + H]⁺; RT: 0.86 min (Method 3) |
| 419 | Starting material: tert-butyl 4-formyl-2,2-dimethylpiperidine-1-carboxylate |
| | MS: m/z 393.2 [M + H]⁺; RT: 0.97 min (Method 3) |
| 420 | Starting material: tert-butyl 2-formyl-6-azaspiro[3.4]octane-6-carboxylate |
| | MS: m/z 391.1 [M + H]⁺; RT: 0.89 min (Method 3) |
| 421 | Starting material: tert-butyl 2-formyl-7-azaspiro[3.5]nonane-7-carboxylate |
| | MS: m/z 405.1 [M + H]+; RT: 0.88 min (Method 3) |
| | ¹H NMR (600 MHz, DMSO-d₆) δ ppm 1.66-1.71 (m, 2 H) 1.77-1.82 (m, 2 |
| | H) 2.25 (m, 2 H) 2.29-2.33 (m, 2 H) 2.41 (s, 3 H) 2.61 (s, 3 H) 2.83-2.88 |
| | (m, 2 H) 2.92-2.97 (m, 2 H) 3.70-3.79 (m, 1 H) 7.66 (m, 1 H) 7.70 (br s, 1 |
| | H) 7.93 (m, 1 H) 8.05 (s, 1 H) 12.36-12.88 (m, 1 H) |
| 422 | Starting material: 1-(cyclopropylmethyl)piperidine-4-carbaldehyde |
| | MS: m/z 419.2 [M + H]+; RT: 1.02 min (Method 3) |
| 423 | Starting material: tert-butyl (3-formylbicyclo[3.2.0]heptan-6-yl)carbamate |
| | MS: m/z 391.3 [M + H]⁺; RT: 0.83 min (Method 3) |
| 424 | Starting material: tert-butyl 7-formyl-2-azaspiro[3.5]nonane-2-carboxylate |
| | MS: m/z 405.1 [M + H]⁺; RT: 0.87 min (Method 3) |
| 559 | Starting material: tert-butyl (7-formylspiro[3.5]nonan-2-yl)carbamate |
| | MS: m/z 419.3 [M + H]⁺; RT: 0.87 min (Method 3) |
| | ¹H NMR (600 MHz, DMSO-d₆) δ ppm 1.45-1.55 (m, 2 H) 1.61-1.69 (m, 1 |
| | H) 1.69-1.80 (m, 2 H) 1.83 (br dd, J = 11.64, 8.20 Hz, 1 H) 1.86-1.93 (m, 3 |
| | H) 1.94-1.99 (m, 1 H) 2.08 (ddd, J = 11.64, 8.01, 4.01 Hz, 1 H) 2.27 (ddd, |
| | J = 11.73, 8.11, 3.81 Hz, 1 H) 2.46 (s, 3 H) 2.55 (s, 1 H) 2.64 (s, 3 H) 2.87 (tt, |
| | J = 11.25, 3.43 Hz, 1 H) 3.69-3.73 (m, 1 H) 7.70 (d, J = 12.21 Hz, 1 H) 7.95 (br |
| | s, 4 H) 8.01 (s, 1 H) 8.20 (br s, 1 H) |
| 425 | Starting material: 1-ethylpiperidine-4-carbaldehyde |
| | MS: m/z 393.2 [M + H]⁺; RT: 0.86 min (Method 3) |
| 426 | Starting material: 1-cyclobutylpiperidine-4-carbaldehyde |
| | MS: m/z 419.3 [M + H]⁺; RT: 0.94 min (Method 3) |
| 427 | Starting material: 1-cyclopentylpiperidine-4-carbaldehyde |
| | MS: m/z 433.3 [M + H]⁺; RT: 1.00 min (Method 3) |
| 428 | Starting material: tert-butyl 3-formyl-8-azabicyclo[3.2.1]octane-8- |
| | carboxylate |
| | MS: m/z 391.1 [M + H]⁺; RT: 0.75 min (Method 3) |
| 429 | Starting material: tert-butyl 6-formyl-3-azabicyclo[3.1.0]hexane-3- |
| | carboxylate |
| | MS: m/z 363.1 [M + H]⁺; RT: 0.75 min (Method 3) |
| 430 | Starting material: 3-methyl-3-azabicyclo[3.1.0]hexane-6-carbaldehyde |
| | MS: m/z 377.0 [M + H]⁺; RT: 1.75 min (Method 8) |
| 574 | Starting material: 8-(difluoromethyl)-2-methyl-6-(4,4,5,5-tetramethyl-1,3,2- |
| | dioxaborolan-2-yl)imidazo[1,2-b]pyridazine |
| | MS: m/z 401.0 [M + H]⁺; RT: 0.82 min (Method 8) |
| 575 | Starting material: 8-methoxy-2-methyl-6-(4,4,5,5-tetramethyl-1,3,2- |
| | dioxaborolan-2-yl)-[1,2,4]triazolo[1,5-b]pyridazine |
| | MS: m/z 382.1 [M + H]⁺; RT: 0.80 min (Method 8) |
| 576 | Starting material: 2,8-dimethyl-6-(4,4,5,5-tetramethyl-1,3,2-dioxaborolan-2- |
| | yl)-[1,2,4]triazolo[1,5-b]pyridazine |
| | MS: m/z 366.1 [M + H]⁺; RT: 0.83 min (Method 8) |

### Example 18—Compounds 131 and 133

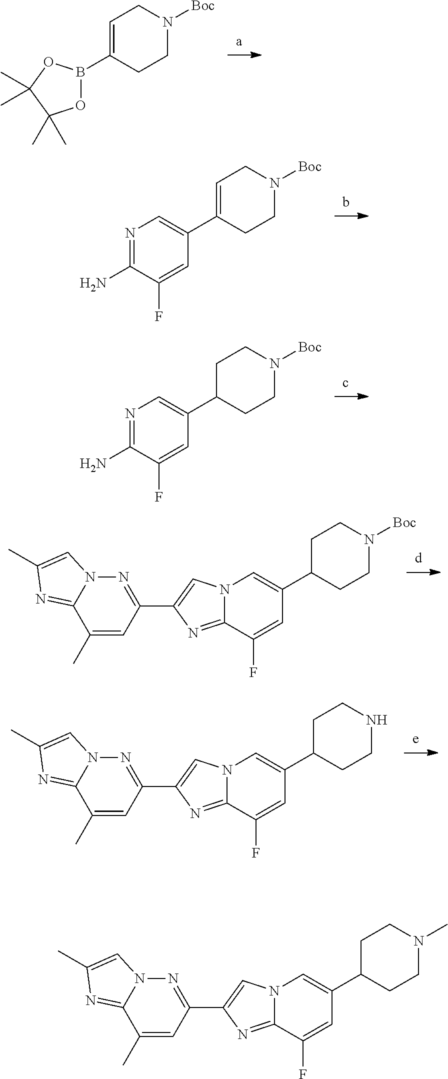

Step a: To a solution of 5-bromo-3-fluoro-pyridin-2-amine (0.5 g, 2.6 mmol) and tert-butyl 4-(4,4,5,5-tetramethyl-1,3,2-dioxaborolan-2-yl)-3,6-dihydro-2H-pyridine-1-carboxylate (809 mg, 2.62 mmol) in dioxane (10 mL) and H₂O (2 mL) was added K₂CO₃ (723 mg, 5.2 mmol) and Pd(dppf)Cl₂ (192 mg, 262 μmol) at 20° C. The mixture was stirred at 90° C. under N₂ for 2 h. The reaction mixture was filtered and concentrated under reduced pressure to give a residue, which was purified by silica column chromatography (PE/EtOAc=1/1 to 0/1) to give tert-butyl 4-(6-amino-5-fluoro-3-pyridyl)-3,6-dihydro-2H-pyridine-1-carboxylate (0.75 g, 2.51 mmol, 95% yield) as a yellow solid. MS: m/z 294.2 [M+H]+

Step b: To a solution of tert-butyl 4-(6-amino-5-fluoro-3-pyridyl)-3,6-dihydro-2H-pyridine-1-carboxylate (0.1 g, 341 umol) in MeOH (10 mL) was added Pd/C (36 mg, 34 μmol, 10% purity) at 20° C. under N₂. The mixture was stirred at 25° C. under 15 psi of H₂ for 2 h. The residue was purified by prep-HPLC (neutral condition) to obtain tert-butyl 4-(6-amino-5-fluoro-3-pyridyl)piperidine-1-carboxylate (0.05 g, 48% yield) as a yellow solid. MS: m/z 296.0 [M+H]⁺

Step c: To a solution of 2-bromo-1-(2,8-dimethylimidazo[1,2-b]pyridazin-6-yl)ethanone (150 mg, 559 umol), tert-butyl 4-(6-amino-5-fluoro-3-pyridyl)piperidine-1-carboxylate (100 mg, 338 μmol) and NaHCO₃ (57 mg, 677 μmol) in t-BuOH (5 mL) at 25° C. The mixture was stirred at 80° C. for 16 hours. The mixture was concentrated in vacuo to give crude product. The crude material was purified on silica gel column chromatography (0-100% PE to EtOAc) to yield tert-butyl 4-[2-(2,8-dimethylimidazo[1,2-b]pyridazin-6-yl)-8-fluoro-imidazo[1,2-a]pyridin-6-yl]piperidine-1-carboxylate (100 mg, 63% yield) as a yellow solid. MS: m/z 465.7 [M+H]⁺

Step d: To a solution of tert-butyl 4-[2-(2,8-dimethylimidazo[1,2-b]pyridazin-6-yl)-8-fluoro-imidazo[1,2-a]pyridin-6-yl]piperidine-1-carboxylate (100 mg, 215 μmol) in 4 M HCL in dioxane (20 mL) at 25° C. The mixture was stirred at 25° C. for 0.5 hour. The mixture was concentrated in vacuo to give crude product. The crude was purified by preparative HPLC (C18-1 150*30 mm*5 um, water (NH₄HCO₃)-ACN as a mobile phase, from 23% to 53%, Gradient Time (min): 15, Flow Rate (ml/min): 25) to give 6-(8-fluoro-6-(piperidin-4-yl)imidazo[1,2-a]pyridin-2-yl)-2,8-dimethylimidazo[1,2-b]pyridazine (30 mg, 37% yield) as a white solid. MS: m/z 365.1 [M+H]⁺; RT 0.64 min (Method 7)

Step e: A solution of 6-[8-fluoro-6-(4-piperidyl)imidazo[1,2-a]pyridin-2-yl]-2,8-dimethyl-imidazo[1,2-b]pyridazine (10 mg, 27.44 μmol), paraformaldehyde (66 mg, 54 μmol, 75 μL) and triethylamine (8 μL, 55 μmol) in 1,2-dichloroethane (5 μL, 55 μmol) and EtOH (5 mL) was stirred at 25° C. for 0.5 hour. Then sodium triacetoxyborohydride (12 mg, 55 μmol) was added and stirred for 1 hour. The mixture was concentrated in vacuo to give crude product which was purified by preparative HPLC (Boston Prime C18 150*30 mm*5 um, water (NH₃H₂O+NH₄HCO₃)-ACN as a mobile phase, from 31% to 61%, Gradient Time (min): 10, Flow Rate (ml/min): 25) to give 6-[8-fluoro-6-(1-methyl-4-piperidyl)imidazo[1,2-a]pyridin-2-yl]-2,8-dimethyl-imidazo[1,2-b]pyridazine (6 mg, 57% yield) as a white solid. MS: m/z 379.1 [M+H]⁺; RT 1.73 min (Method 7)

Using the procedure described for Example 19 above, additional compounds described herein were prepared by substituting the appropriate boronic ester starting material in step a, aldehyde in step e, suitable reagents and reaction conditions, obtaining compounds such as those selected from:

| Cpd | Data |
|---|---|
| 132 | Starting material: tert-butyl 4-(4,4,5,5-tetramethyl-1,3,2-dioxaborolan-2- |
| | yl)-3,6-dihydropyridine-1(2H)-carboxylate and acetaldehyde |
| | MS: m/z 393.2 [M + H]⁺; RT: 1.74 min (Method 7) |
| 134 | Starting material: tert-butyl 5-(4,4,5,5-tetramethyl-1,3,2-dioxaborolan-2- |
| | yl)-3,6-dihydropyridine-1(2H)-carboxylate |
| | MS: m/z 365.1 [M + H]⁺; RT: 0.82 min (Method 7) |
| 130 | Starting material: tert-butyl 3-(4,4,5,5-tetramethyl-1,3,2-dioxaborolan-2- |
| | yl)-6-azabicyclo[3.1.1]hept-2-ene-6-carboxylate |
| | MS: m/z 391.2 [M + H]⁺; RT: 1.789 min (Method 7) |

### Example 19—Compound 127

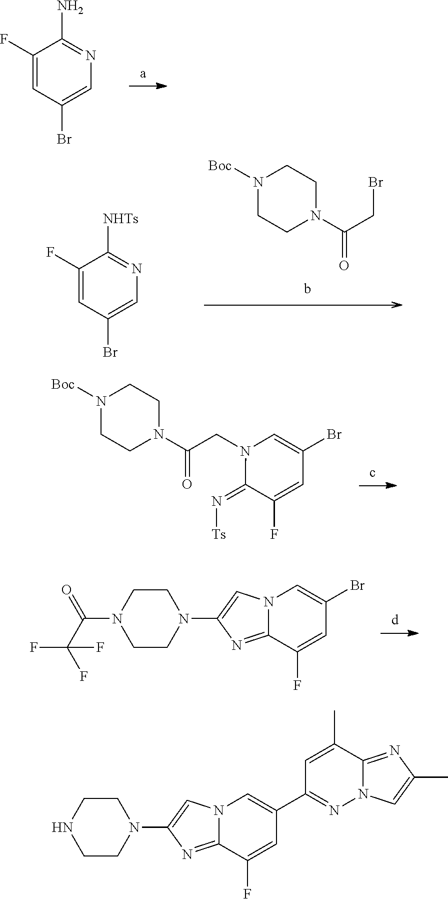

Step a: To a mixture of 5-bromo-3-fluoropyridin-2-amine (5 g, 26.2 mmol) and pyridine (2.1 g, 26.2 mmol, 2 mL) was added 4-methylbenzene-1-sulfonyl chloride (5 g, 26.2 mmol) at 0° C., the reaction mixture was stirred at 100° C. for 12 h under N₂ atmosphere. The mixture was concentrated and then diluted with water (300 mL), extracted with EtOAc and concentrated to give N-(5-bromo-3-fluoropyridin-2-yl)-4-methylbenzenesulfonamide (6.9 g, 20 mmol, 76% yield) as an off-white solid. MS: m/z 345 [M+H]⁺; RT 0.78 min (Method 7).

Step b: To a solution of tert-butyl 4-(2-bromoacetyl)piperazine-1-carboxylate (98 mg, 319 μmol) and N-(5-bromo-3-fluoro-2-pyridyl)-4-methyl-benzenesulfonamide (100 mg, 290 μmol) in DMF (2 mL) was added NaHCO₃ (50 mg, 594 μmol). The mixture was stirred at 100° C. for 16 h. The reaction mixture was concentrated in vacuum to give crude product. The crude product was purified by prep-HPLC (Column: Boston Green ODS 150*30 mm*5 um; Condition: water (FA)-ACN, Begin B 65, End B 95; Gradient Time (min): 12; 100% B Hold Time (min): 2; Flow Rate (ml/min): 25) to give tert-butyl 4-[2-[(2E)-5-bromo-3-fluoro-2-(p-tolylsulfonylimino)-1-pyridyl]acetyl]piperazine-1-carboxylate (50 mg, 30% yield) as a yellow solid. MS: m/z 571 [M+H]⁺; RT 0.692 min (Method 7).

Step c: To a solution of tert-butyl 4-[2-[(2E)-5-bromo-3-fluoro-2-(p-tolylsulfonylimino)-1-pyridyl]acetyl]piperazine-1-carboxylate (35 mg, 61 μmol) in DCM (2 mL) was added TFAA (3 g, 14 mmol, 2 mL) and the mixture was stirred at 40° C. for 16 h. The mixture was concentrated in vacuo to give the residue, which was purified by prep-HPLC (Boston Green ODS 150*30 mm*5 um; Condition: water (FA)-ACN, Begin B 49, End B 59; Gradient Time (min): 12; 100% B Hold Time (min): 2; Flow Rate (ml/min): 25) to give 1-[4-(6-bromo-8-fluoro-imidazo[1,2-a]pyridin-2-yl)piperazin-1-yl]-2,2,2-trifluoro-ethanone (20 mg, 83% yield) as a white solid.

Step d: To a solution of 1-[4-(6-bromo-8-fluoro-imidazo[1,2-a]pyridin-2-yl)piperazin-1-yl]-2,2,2-trifluoro-ethanone (20 mg, 50 μmol) in dioxane (3 mL) and water (0.5 mL) was added 2,8-dimethyl-6-(4,4,5,5-tetramethyl-1,3,2-dioxaborolan-2-yl)imidazo[1,2-b]pyridazine (14 mg, 51 μmol), K₂CO₃ (21 mg, 152 μmol) and Pd(dppf)Cl₂ (4 mg, 5 μmol) under N₂. The mixture was stirred at 80° C. for 2 h. The mixture was concentrated and then water (50 mL) was added. The mixture was extracted with EtOAc (20 mL×3). The combined organic layers were washed with brine (30 mL), dried over Na₂SO₄, filtered and concentrated in vacuo to give the residue, which was purified by prep-HPLC (Column: Boston Prime C18 150*30 mm*5 um; Condition: water (NH₃H₂O+NH₄HCO₃)-ACN, Begin B 44, End B 74; Gradient Time (min): 10; 100% B Hold Time (min): 2; Flow Rate (ml/min): 25) to obtain 6-(8-fluoro-2-piperazin-1-yl-imidazo[1,2-a]pyridin-6-yl)-2,8-dimethyl-imidazo[1,2-b]pyridazine (8 mg, 44% yield). MS: m/z 366.0 [M+H]⁺; RT 1.2 min (Method 7).

### Example 20—Compound 150

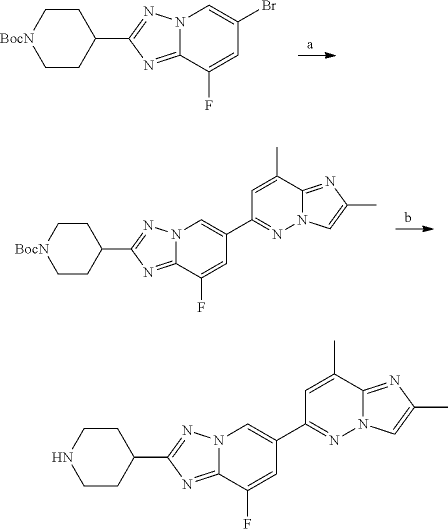 [SMILES: CC(C)(C)OC(=O)N1CCC(c2nc3c(F)cc(Br)cn3n2)CC1 ; Cc1cn2nc(-c3cc(F)c4nc(C5CCN(C(=O)OC(C)(C)C)CC5)nn4c3)cc(C)c2n1 ; Cc1cn2nc(-c3cc(F)c4nc(C5CCNCC5)nn4c3)cc(C)c2n1]

Step a: A mixture of tert-butyl 4-(6-bromo-8-fluoro-[1,2,4]triazolo[1,5-a]pyridin-2-yl)piperidine-1-carboxylate (60 mg, 150.3 μmol), 2,8-dimethyl-6-(4,4,5,5-tetramethyl-1,3,2-dioxaborolan-2-yl)imidazo[1,2-b]pyridazine (53.4 mg, 195.36 μmol), Pd(dppf)Cl₂ DCM (12.27 mg, 15.03 μmol), dicesium carbonate (146.8 mg, 450.8 μmol) in dioxane (1.2 mL) and water (300 μL) was stirred under N₂ at 90° C. for 4 h. The mixture was filtered through celite/MgSO₄ (DCM/EtOAc eluent), concentrated, and used crude in the next reaction (assumed 100% yield). MS: m/z 466.3 [M+H]⁺

Step b: A mixture of tert-butyl 4-[6-(2,8-dimethylimidazo[1,2-b]pyridazin-6-yl)-8-fluoro-[1,2,4]triazolo[1,5-a]pyridin-2-yl]piperidine-1-carboxylate (70 mg, 150.37 μmol) and HCl (4 M in dioxane, 300 μL), in DCM (3.0 mL) was stirred at rt for 3 h. The mixture was concentrated and then purified by preparative HPLC (Column: Sunfire C18 100×19 mm, 5 mm; Mobile phase A: MeCN; Mobile phase B: H2O; Modifier: 0.1% TFA) to obtain 6-(2,8-dimethylimidazo[1,2-b]pyridazin-6-yl)-8-fluoro-2-(4-piperidyl)-[1,2,4]triazolo[1,5-a]pyridine (16.4 mg, 23% yield). MS: m/z 366.2 [M+H]⁺; RT: 0.38 min (Method 4)

Using the procedure described for Example 20 above, additional compounds described herein were prepared by substituting the appropriate boronic ester in step a, suitable reagents, and reaction conditions, obtaining compounds such as those selected from:

| Cpd ID | Data |
|---|---|
| 151 | Starting material: 8-fluoro-2-methyl-6-(4,4,5,5-tetramethyl-1,3,2- |
| | dioxaborolan-2-yl)imidazo[1,2-a]pyridine |
| | MS: m/z 369.1 [M + H]⁺; RT: 0.33 min (Method 4) |
| 152 | Starting material: 1-ethyl-3-methyl-7-(4,4,5,5-tetramethyl-1,3,2- |
| | dioxaborolan-2-yl)pyrrolo[1,2-a]pyrazine |
| | MS: m/z 379.2 [M + H]⁺; RT: 0.42 min (Method 4) |
| 567 | Starting material: 8-(difluoromethyl)-2-methyl-6-(4,4,5,5-tetramethyl-1,3,2- |
| | dioxaborolan-2-yl)imidazo[1,2-b]pyridazine |
| | MS: m/z 402.1 [M + H]⁺; RT: 0.27 min (Method 9) |
| 568 | Starting material: 2,8-dimethyl-6-(4,4,5,5-tetramethyl-1,3,2-dioxaborolan-2- |
| | yl)-[1,2,4]triazolo[1,5-b]pyridazine |
| | MS: m/z 367.2 [M + H]⁺; RT: 0.27 min (Method 9) |

### Example 21—Compound 294 and/or 264

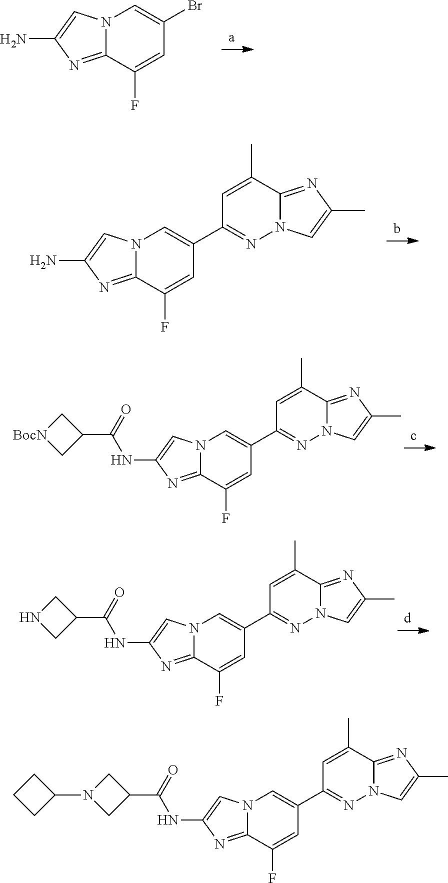 [SMILES: Nc1cn2cc(Br)cc(F)c2n1 ; Cc1cn2nc(-c3cc(F)c4nc(N)cn4c3)cc(C)c2n1 ; Cc1cn2nc(-c3cc(F)c4nc(NC(=O)C5CN(C(=O)OC(C)(C)C)C5)cn4c3)cc(C)c2n1 ; Cc1cn2nc(-c3cc(F)c4nc(NC(=O)C5CNC5)cn4c3)cc(C)c2n1 ; Cc1cn2nc(-c3cc(F)c4nc(NC(=O)C5CN(C6CCC6)C5)cn4c3)cc(C)c2n1]

Step a: To a solution of 6-bromo-8-fluoro-imidazo[1,2-a]pyridin-2-amine (200 mg, 869 μmol) in dioxane (20 mL) and water (1 mL) was added 2,8-dimethyl-6-(4,4,5,5-tetramethyl-1,3,2-dioxaborolan-2-yl)imidazo[1,2-b]pyridazine (237 mg, 869 μmol), Pd(dppf)Cl2 (64 mg, 87 μmol) and K₂CO₃ (360 mg, 2.61 mmol) under N₂. The mixture was stirred at 90° C. for 2h. The mixture was concentrated and then water (80 mL) was added. The mixture was extracted with EtOAc (50 mL×3). The combined organic layers were washed with brine (50 mL), dried over Na₂SO₄, filtered, and concentrated in vacuo to give the crude product. The crude product was purified by Combi-Flash (DCM/MeOH=10/1) to yield 6-(2,8-dimethylimidazo[1,2-b]pyridazin-6-yl)-8-fluoro-imidazo[1,2-a]pyridin-2-amine (200 mg, 674.99 μmol, 77.64% yield) as brown solid. MS: m/z 297.2 [M+H]⁺; RT: 0.1237 min (Method 10)

Step b: To a solution of 6-(2,8-dimethylimidazo[1,2-b]pyridazin-6-yl)-8-fluoro-imidazo[1,2-a]pyridin-2-amine (400 mg, 1.35 mmol) in Pyridine (10 mL) was added 1-tert-butoxycarbonylazetidine-3-carboxylic acid (326 mg, 1.62 mmol) and EDCI (388 mg, 2 mmol) and the mixture was stirred at 90° C. for 1 h. The mixture was then concentrated and water (80 mL) was added. The mixture was extracted with EtOAc (50 mL×3). The combined organic layers were washed with brine (50 mL), dried over Na₂SO₄, filtered and concentrated in vacuo to give the residue, which was purified by Combi-Flash (DCM/MeOH=20/1) to give tert-butyl 3-[[6-(2,8-dimethylimidazo[1,2-b]pyridazin-6-yl)-8-fluoro-imidazo[1,2-a]pyridin-2-yl]carbamoyl]azetidine-1-carboxylate (250 mg, 521 umol, 39% yield) as yellow solid. MS: m/z 480.3 [M+H]⁺; RT: 0.798 min (Method 4)

Step c: To a solution of tert-butyl 3-[[6-(2,8-dimethylimidazo[1,2-b]pyridazin-6-yl)-8-fluoro-imidazo[1,2-a]pyridin-2-yl]carbamoyl]azetidine-1-carboxylate (50 mg, 104 umol) in DCM (2 mL) was added TFA (2 mL) and the mixture was stirred at 20° C. for 1 h. The mixture was concentrated in vacuo, which was purified by prep-HPLC (FA) to give N-[6-(2,8-dimethylimidazo[1,2-b]pyridazin-6-yl)-8-fluoro-imidazo[1,2-a]pyridin-2-yl]azetidine-3-carboxamide (22 mg, 57 umol, 55% yield) as white solid. MS: m/z 380.0 [M+H]⁺; RT: 0.777 min (Method 7)

Step d: To a solution of N-[6-(2,8-dimethylimidazo[1,2-b]pyridazin-6-yl)-8-fluoro-imidazo[1,2-a]pyridin-2-yl]azetidine-3-carboxamide (30 mg, 79 umol) and cyclobutanone (17 mg, 237 umol, 18 uL) in DCE/EtOH (4 mL) and stirred at 20° C. for 20 min. The reaction mixture was added sodium triacetoxyboro hydride (50 mg, 237 umol) and stirred at 20° C. for 12 h. The reaction was filtered and concentrated under reduced pressure to give the residue. The residue was purified by preparative HPLC to give the 1-cyclobutyl-N-[6-(2,8-dimethylimidazo[1,2-b]pyridazin-6-yl)-8-fluoro-imidazo[1,2-a]pyridin-2-yl]azetidine-3-carboxamide (4.6 mg, 10 umol, 13% yield, 97% purity) as a yellow solid. MS: m/z 434.1 [M+H]⁺; RT: 1.23 min (Method 8); 1H NMR: (400 MHz, METHANOL-d4) δ ppm=9.05 (s, 1H), 8.34 (d, J=2.8 Hz, 1H), 7.94 (s, 1H), 7.81 (d, J=12.4 Hz, 1H), 7.60 (s, 1H), 3.73-3.68 (m, 2H), 3.56-3.48 (m, 3H), 2.68 (s, 3H), 2.64-2.58 (m, 1H), 2.49 (s, 3H), 1.01 (d, J=6.4 Hz, 6H).

Using the procedure described for Example 21 above, additional compounds described herein were prepared by substituting the appropriate boronic ester in step a, suitable reagents, and reaction conditions, obtaining compounds such as those selected from:

| Cpd ID | Data |
|---|---|
| 128 | Starting material: 3-(dimethylamino)cyclopentane-1-carboxylic acid |
| | MS: m/z 436.1 [M + H]⁺; RT: 0.91 min (Method 3) |
| | 1H NMR (500 MHz, METHANOL-d4) δ ppm 1.93-2.03 (m, 2 H) 2.07-2.23 |
| | (m, 3 H) 2.24-2.32 (m, 1 H) 2.43-2.51 (m, 1 H) 2.61 (s, 3 H) 2.76 (s, 3 H) |
| | 2.98 (br d, J = 7.93 Hz, 7 H) 3.67-3.78 (m, 1 H) 7.84-7.91 (m, 1 H) 7.96- |
| | 8.02 (m, 1 H) 8.19 (br s, 1 H) 8.33-8.39 (m, 1 H) 9.13-9.19 (m, 1 H) |
| 291 | Starting material: 1-tert-butoxycarbonylazetidine-3-carboxylic acid |
| | MS: m/z 425.2 [M + H]+; RT: 1.090 min (Method 10) |
| 289 | Starting material: 3-(tert-butoxycarbonylamino)cyclobutanecarboxylic acid |
| | MS: m/z 422.2 [M + H]⁺; RT: 0.741 min (Method 10) |
| | 1H NMR: (400 MHz, METHANOL-d4) δ ppm = 9.00 (s, 1H), 8.29 (d, J = 3.2 |
| | Hz, 1H), 7.92 (s, 1H), 7.80-7.70 (m, 1H), 7.58 (s, 1H), 3.00-2.90 (m, 1H), |
| | 2.85-2.75 (m, 1H), 2.67 (s, 3H), 2.50-2.40 (m, 5H), 2.25-2.20 (m, 2H), 2.20 |
| | (s, 6H). |
| 288 | Starting material: 3-(tert-butoxycarbonylamino)cyclobutanecarboxylic acid |
| | MS: m/z 422.1 [M + H]⁺; RT: 0.719 min (Method 10) |
| 287 | Starting material: 6-(2,8-dimethylimidazo[1,2-b]pyridazin-6-yl)-8-fluoro- |
| | imidazo[1,2-a]pyridin-2-amine |
| | MS: m/z 394.3 [M + H]⁺; RT: 0.645 min (Method 7) |
| | 1H NMR (400 MHz, METHANOL-d4) δ ppm = 9.05 (s, 1H), 8.40 (d, J = 2.8 |
| | Hz, 1H), 8.04 (s, 1H), 7.84 (d, J = 12.0 Hz, 1H), 7.77 (s, 1H), 4.77-4.69 (m, |
| | 1H), 4.26 (dd, J = 10.8, 8.0 Hz, 1H), 4.13 (t, J = 9.6 Hz, 1H), 3.68-2.61 (m, 1 |
| | H) 2.71 (s, 3H), 2.54 (s, 3H), 1.67 (d, J = 6.8 Hz, 3H). |
| 286 | Starting material: 6-(2,8-dimethylimidazo[1,2-b]pyridazin-6-yl)-8-fluoro- |
| | imidazo[1,2-a]pyridin-2-amine |
| | MS: m/z 394.1 [M + H]⁺; RT: 1.265 min (Method 10) |
| | 1H NMR: (400 MHz, METHANOL-d4) δ ppm = 9.09 (d, J = 1.2 Hz, 1H), 8.40 |
| | (d, J = 2.8 Hz, 1H), 8.04 (s, 1H), 7.84 (d, J = 12.0 Hz, 1H), 7.77 (s, 1H), 4.77- |
| | 4.69 (m, 1H), 4.26 (dd, J = 10.8, 8.0 Hz, 1H), 4.13 (t, J = 9.6 Hz, 1H), 3.68- |
| | 2.61 (m, 1 H) 2.71 (s, 3H), 2.54 (s, 3H), 1.67 (d, J = 6.8 Hz, 3H). |
| 285 | Starting material: (1-ethoxycyclopropoxy)-trimethyl-silane |
| | MS: m/z 448.2 [M + H]⁺; RT: 1.413 min (Method 10) |
| 284 | Starting material: 2-(1-(tert-butoxycarbonyl)piperidin-4-yl)acetic acid |
| | MS: m/z 422.2 [M + H]⁺; RT: 1.668 min (Method 8) |
| 283 | Starting material: rac-(R)-2-(1-(tert-butoxycarbonyl)pyrrolidin-3-yl)acetic |
| | acid |
| | MS: m/z 408.2 [M + H]⁺; RT: 0.820 min (Method 10) |
| 282 | Starting material: (1-ethoxycyclopropoxy)-trimethyl-silane |
| | MS: m/z 434.3 [M + H]⁺; RT: 1.735 min (Method 10) |
| 281 | Starting material: 2-(1-(tert-butoxycarbonyl)azetidin-3-yl)acetic acid |
| | MS: m/z 394.1 [M + H]⁺; RT: 1.297 min (Method 10) |
| 280 | Starting material: rac-(1R,4S,5S)-2-(tert-butoxycarbonyl)-2- |
| | azabicyclo[2.2.1]heptane-5-carboxylic acid |
| | MS: m/z 420.1 [M + H]⁺; RT: 1.340 min (Method 3min) |
| | 1H NMR: (400 MHz, METHANOL-d4) δ ppm = 9.04 (s, 1H), 8.52 (s, 1H), |
| | 8.30 (d, J = 3.2 Hz, 1H), 7.94 (s, 1H), 7.82 (d, J = 10.8 Hz, 1H), 7.60 (s, 1H), |
| | 4.20-4.17 (m, 1H), 3.26-3.21 (m, 1H), 3.16-3.13 (m, 1H), 2.99-2.96 (m, 1H), |
| | 2.93-2.89 (m, 1H), 2.68 (s, 3H), 2.49 (s, 3H), 2.38-2.31 (m, 1H), 2.14-2.04 (m, |
| | 2H), 1.79-1.72 (m, 1H). |
| 279 | Starting material: rac-(5R)-1-azabicyclo[3.2.1]octane-5-carboxylic acid |
| | MS: m/z 434.2 [M + H]⁺; RT: 1.362 min (Method 10) |
| 278 | Starting material: 1-(tert-butoxycarbonyl)piperidine-3-carboxylic acid |
| | MS: m/z 422.0 [M + H]⁺; RT: 2.611 min (Method 8) |
| 277 | Starting material: 1-(tert-butoxycarbonyl)piperidine-3-carboxylic acid |
| | MS: m/z 422.2 [M + H]⁺; RT: 0.617 min (Method 10) |
| 276 | Starting material: 2-(tert-butoxycarbonyl)-2-azaspiro[3.3 ]heptane-6- |
| | carboxylic acid |
| | MS: m/z 420.1 [M + H]⁺; RT: 1.340 min (Method 10) |
| 246 | Starting material: (1RS,5SR,6r)-3-(tert-butoxycarbonyl)-3- |
| | azabicyclo[3.1.0]hexane-6-carboxylic acid |
| | MS: m/z 406.1 [M + H]⁺; RT: 0.87 min (Method 3) |
| 247 | Starting material: rac-(1R,3R)-3-(dimethylamino)cyclohexane-1-carboxylic |
| | acid |
| | MS: m/z 368.1 [M + H]⁺; RT: 0.87 min (Method 3) |
| 248 | Starting material: 1-(tert-butoxycarbonyl)piperidine-3-carboxylic acid |
| | MS: m/z 450.1 [M + H]⁺; RT: 0.99 min (Method 3) |
| 249 | Starting material: 3-((tert-butoxycarbonyl)amino)cyclobutane-1-carboxylic |
| | acid |
| | MS: m/z 408.1 [M + H]⁺; RT: 0.89 min (Method 3) |
| 250 | Starting material: 1-(tert-butoxycarbonyl)piperidine-4-carboxylic acid |
| | MS: m/z 408.1 [M + H]⁺; RT: 0.87 min (Method 3) |
| 251 | Starting material: 3-((dimethylamino)methyl)cyclobutane-1-carboxylic acid |
| | MS: m/z 436.1 [M + H]⁺; RT: 0.67 min (Method 3) |
| 252 | Starting material: 6-(tert-butoxycarbonyl)-6-azaspiro[3.4]octane-2-carboxylic |
| | acid |
| | MS: m/z 434.1 [M + H]⁺; RT: 0.92 min (Method 3) |
| 253 | Starting material: rac-(1R,3S)-5-(tert-butoxycarbonyl)-5- |
| | azaspiro[2.4]heptane-1-carboxylic acid |
| | MS: m/z 420.1 [M + H]⁺; RT: 0.91 min (Method 3) |
| 254 | Starting material: 1-(tert-butoxycarbonyl)azetidine-3-carboxylic acid |
| | MS: m/z 436.1 [M + H]⁺; RT: 0.84 min (Method 3) |
| 255 | Starting material: 2-(1-(tert-butoxycarbonyl)piperidin-3-yl)acetic acid |
| | MS: m/z 368.1 [M + H]⁺; RT: 0.87 min (Method 3) |
| 256 | Starting material: oxetane-3-carboxylic acid |
| | MS: m/z 381.0 [M + H]⁺; RT: 1.690 min (Method 10) |
| 257 | Starting material: 1-(tert-butoxycarbonyl)azetidine-3-carboxylic acid |
| | MS: m/z 368.1 [M + H]⁺; RT: 0.87 min (Method 3) |
| 258 | Starting material: 1-(tert-butoxycarbonyl)-3-methylazetidine-3-carboxylic |
| | acid |
| | MS: m/z 394.2 [M + H]⁺; RT: 1.363 min (Method 8) |
| 259 | Starting material: rac-(R)-1-(tert-butoxycarbonyl)-2,2-dimethylazetidine-3- |
| | carboxylic acid |
| | MS: m/z 368.1 [M + H]⁺; RT: 0.87 min (Method 3) |
| 260 | Starting material: Starting material: rac-(R)-1-(tert-butoxycarbonyl)-2,2- |
| | dimethylazetidine-3-carboxylic acid |
| | MS: m/z 422.2 [M + H]⁺; RT: 1.263 min (Method 10) |
| 261 | Starting material: 1-(tert-butoxycarbonyl)-3-methylazetidine-3-carboxylic |
| | acid |
| | MS: m/z 408.2 [M + H]⁺; RT: 2.940 min (Method 8) |
| 262 | Starting material: (1R,5S,6r)-3-((tert-butoxycarbonyl)-12-azaneyl)-3- |
| | azabicyclo[3.1.0]hexane-6-carboxylic acid |
| | MS: m/z 420.2 [M + H]⁺; RT: 1.649 min (Method 10) |
| | 1H NMR: (500 MHz, METHANOL-d4) δ ppm = 8.96 (d, J = 1.0 Hz, 1 H), |
| | 8.47 (s, 0.5 H), 8.17 (d, J = 3.0 Hz, 1 H), 7.89 (s, 1 H), 7.75 (d, J = 12.0 Hz, 1 |
| | H), 7.54 (d, J = 1.0 Hz, 1 H), 3.30-3.20 (m, 2 H), 2.80-2.70 (m, 2 H), 2.65 (s, |
| | 3 H), 2.52 (s, 3 H), 2.47 (s, 3 H), 2.13 (s, 3 H). |
| 263 | Starting material: 1-(tert-butoxycarbonyl)azetidine-3-carboxylic acid |
| | MS: m/z 434.1 [M + H]⁺; RT: 1.226 min (Method 7) |
| 265 | Starting material: (1-ethoxycyclopropoxy)-trimethyl-silane |
| | MS: m/z 420.2 [M + H]⁺; RT: 1.628 min (Method 10) |
| 266 | Starting material: 1-(tert-butoxycarbonyl)azetidine-3-carboxylic acid |
| | MS: m/z 453.2 [M + H]⁺; RT: 1.355 min (Method 10) |
| 267 | Starting material: 1-(tert-butoxycarbonyl)azetidine-3-carboxylic acid |
| | MS: m/z 444.1 [M + H]⁺; RT: 1.307 min (Method 10) |
| 268 | Starting material: 4-(tert-butoxycarbonyl)-4-azaspiro[2.5]octane-7-carboxylic |
| | acid |
| | MS: m/z 434.2 [M + H]⁺; RT: 1.639 min (Method 10) |
| 269 | Starting material: 1-(tert-butoxycarbonyl)piperidine-4-carboxylic acid |
| | MS: m/z 422.2 [M + H]⁺; RT: 1.663 min (Method 10) |
| | 1H NMR: (400 MHz, METHANOL-d4) δ ppm = 8.98 (s, 1H), 8.51 (s, 1H), |
| | 8.26 (d, J = 2.8 Hz, 1H), 7.89 (s, 1H), 7.75 (d, J = 12.0 Hz, 1H), 7.54 (s, 1H), |
| | 3.43 (d, J = 10.4 Hz, 2H), 2.89-2.76 (m, 3H), 2.74 (s, 3H), 2.65 (s, 3H), 2.47 |
| | (s, 3H), 2.15-2.01 (m, 4H). |
| 270 | Starting material: 1-(tert-butoxycarbonyl)-3-fluoroazetidine-3-carboxylic |
| | acid |
| | MS: m/z 398.2 [M + H]⁺; RT: 1 min (Method 10) |
| 271 | Starting material: 2-(tert-butoxycarbonyl)-2-azabicyclo[4.1.0]heptane-5- |
| | carboxylic acid |
| | MS: m/z 420.2 [M + H]⁺; RT: 1.35 min (Method 3) |
| 272 | Starting material: 3-(tert-butoxycarbonyl)-3-azabicyclo[3.1.1]heptane-1- |
| | carboxylic acid |
| | MS: m/z 420.2 [M + H]⁺; RT: 0.617 min (Method 7) |
| 273 | Starting material: 2-(1-(tert-butoxycarbonyl)pyrrolidin-2-yl)acetic acid |
| | MS: m/z 408.1 [M + H]⁺; RT: 1.634 min (Method 8) |
| | 1H NMR: (500 MHz, METHANOL-d4) δ ppm = 9.01 (s, 1 H), 8.51 (s, 1 H), |
| | 8.31 (d, J = 3.0 Hz, 1 H), 7.92 (s, 1 H), 7.80 (d, J = 12.0 Hz, 1 H), 7.57 (d, |
| | J = 1.0 Hz, 1 H), 4.01-3.93 (m, 1 H), 3.36-3.32 (m, 2 H), 3.10-3.06 (m, 1 H), |
| | 2.93-2.88 (m, 1 H), 2.67 (s, 3 H), 2.49 (s, 3 H), 2.34-2.31 (m, 1 H), 2.17-2.09 |
| | (m, 1 H), 2.08-1.99 (m, 1 H), 1.83-1.73 (m, 1 H). |
| 274 | Starting material: 2-(1-(tert-butoxycarbonyl)piperidin-2-yl)acetic acid |
| | MS: m/z 422.2 [M + H]⁺; RT: 1.689 min (Method 8) |
| | 1H NMR: (500 MHz, METHANOL-d4) δ ppm = 8.99 (s, 1 H), 8.54 (s, 1 H), |
| | 8.29 (d, J = 3.0 Hz, 1 H), 7.90 (s, 1 H), 7.77 (d, J = 12.0 Hz, 1 H), 7.55 (d, |
| | J = 1.0 Hz, 1 H), 3.61-3.54 (m, 1 H), 3.43-3.41 (m , 1 H), 3.08-3.02 (m, 1 H), |
| | 2.89-2.77 (m, 2 H), 2.67-2.65 (m, 3 H), 2.48 (s , 3 H), 2.00-1.93 (m, 1 H), |
| | 1.96-1.89 (m, 2 H), 1.72-1.56 (m, 3 H). |
| 275 | Starting material: 2-(piperidin-1-yl)acetic acid |
| | MS: m/z 422.2 [M + H]⁺; RT: 1.358 min (Method 10) |
| 296 | Starting material: 6-chloro-8-methoxy-2-methyl-[1,2,4]triazolo[1,5- |
| | b]pyridazine |
| | MS: m/z 438.47 [M + H]⁺; RT: 1.449 min (Method 10) |
| | 1H NMR: (500 MHz, METHANOL-d4) δ ppm = 9.16 (s, 1 H), 8.46 (s, 1 H), |
| | 8.37 (d, J = 2.5 Hz, 1 H), 7.86 (d, J = 12.0 Hz, 1 H), 7.52 (s, 1 H), 4.27 (s, 3 |
| | H), 4.19-4.05 (m, 4 H), 3.73 (t, J = 7.5 Hz, 1 H), 3.22-3.19 (m, 1 H), 2.60 |
| | (s, 3 H), 1.19 (d, J = 6.4 Hz, 6 H). |
| 566 | Starting material: 6-chloro-2,8-dimethyl-[1,2,4]triazolo[1,5-b]pyridazine |
| | MS: m/z 465.2 [M + H]⁺; RT: 0.52 min (Method 10) |

### Example 22—Compound 126

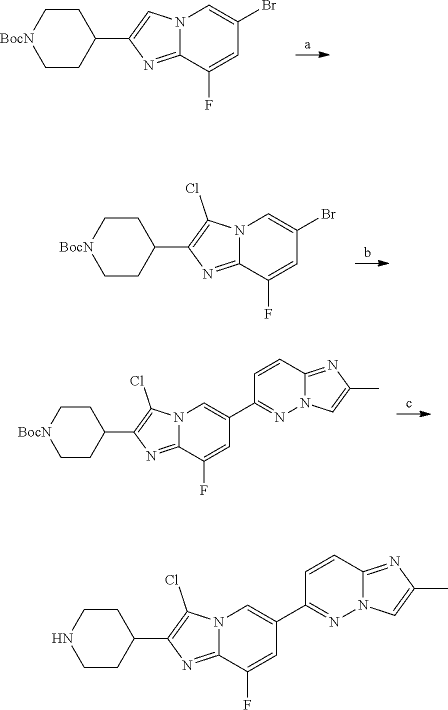 [SMILES: CC(C)(C)OC(=O)N1CCC(c2cn3cc(Br)cc(F)c3n2)CC1 ; CC(C)(C)OC(=O)N1CCC(c2nc3c(F)cc(Br)cn3c2Cl)CC1 ; Cc1cn2nc(-c3cc(F)c4nc(C5CCN(C(=O)OC(C)(C)C)CC5)c(Cl)n4c3)ccc2n1 ; Cc1cn2nc(-c3cc(F)c4nc(C5CCNCC5)c(Cl)n4c3)ccc2n1]

Step a: Tert-butyl 4-(6-bromo-8-fluoro-imidazo[1,2-a]pyridin-2-yl)piperidine-1-carboxylate (100 mg, 251 μmol) was dissolved in DCM (2.5 mL), NCS (34 mg, 251 μmol) was added and this was stirred at room temp for 30 minutes. The resulting was purified by silica gel flash chromatography using a gradient of 0-100% EtOAc/heptane to afford tert-butyl 4-(6-bromo-3-chloro-8-fluoro-imidazo[1,2-a]pyridin-2-yl)piperidine-1-carboxylate (67 mg, 61% yield) MS: m/z 432 [M+H]+.

Step b: Tert-butyl 4-(6-bromo-3-chloro-8-fluoro-imidazo[1,2-a]pyridin-2-yl)piperidine-1-carboxylate (39.5 mg, 91.24 μmol) was dissolved in dioxane (1.5 mL) and water (0.8 mL) with 2-methyl-6-(4,4,5,5-tetramethyl-1,3,2-dioxaborolan-2-yl)imidazo[1,2-b]pyridazine (26.0 mg, 100.4 μmol), cesium carbonate (59.5 mg, 182.4 μmol) and di-tert-butyl(cyclopentyl)phosphane; dichloropalladium; iron (5.9 mg, 9.12 μmol). The mixture was degassed with N₂ and heated in the microwave to 90° C. for 2 h. The resulting mixture was cooled to room temp and purified by silica gel flash chromatography using a gradient of 0-100% EtOAc/heptane. MS: m/z 485 [M+H]⁺.

Step c: Tert-butyl 4-[3-chloro-8-fluoro-6-(2-methylimidazo[1,2-b]pyridazin-6-yl)imidazo[1,2-a]pyridin-2-yl]piperidine-1-carboxylate (5.6 mg, 12 μmol) was dissolved in DCM (1 mL) and HCl (4 M in dioxane, 57 μL) was added and stirred at room temp for 1 h. The resulting mixture was concentrated to dryness to afford 6-[3-chloro-8-fluoro-2-(4-piperidyl)imidazo[1,2-a]pyridin-6-yl]-2-methyl-imidazo[1,2-b]pyridazine (4 mg, 90% yield). ¹H NMR (500 MHz, METHANOL-d4) δ ppm 2.00-2.18 (m, 4H) 2.57 (s, 3H) 3.06-3.16 (m, 3H) 3.25-3.32 (m, 1H) 3.43-3.52 (m, 4H) 7.92 (br d, J=11.60 Hz, 1H) 8.32 (s, 1H) 8.36-8.47 (m, 2H) 8.87-8.93 (m, 1H) MS: m/z 385 [M+H]⁺.

### Example 23—Compound 154 & 456

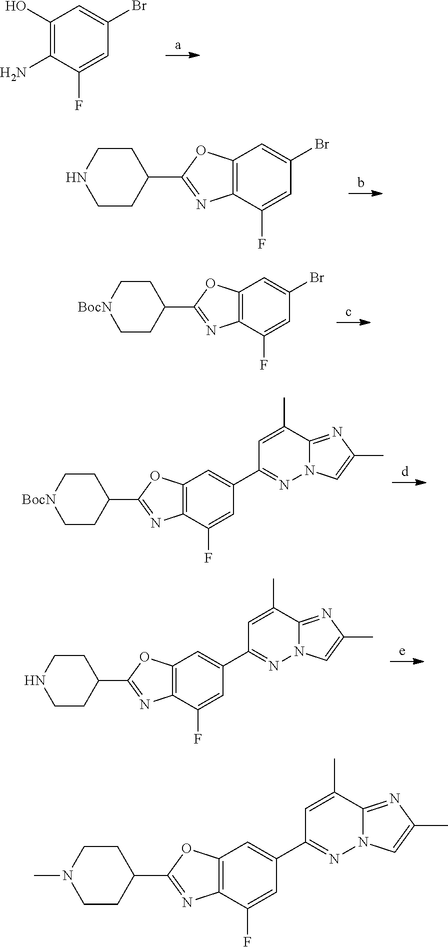

Step a: A mixture of 2-amino-5-bromo-3-fluoro-phenol (360 mg, 1.75 mmol) and 1-tert-butoxycarbonylpiperidine-4-carboxylic acid (400.6 mg, 1.75 mmol) in PPA (3 mL) was stirred at 160° C. for 0.5 h. No further purification as it is used for the next step directly. [M+2+H]=301.0

Step b: A solution of 6-bromo-4-fluoro-2-(4-piperidyl)-1,3-benzoxazole (400 mg, 1.34 mmol) and TEA (1.34 mmol, 186 μL), tert-butoxycarbonyl tert-butyl carbonate (291.8 mg, 1.34 mmol, 307.2 μL) in THF (15 mL) and water (40 mL) was stirred at 0° C. for 0.5 h. The mixture was concentrated and then water (50 mL) was added. The mixture was extracted with EtOAc (50 mL×3). The combined organic layers were dried over Na₂SO₄, filtered and concentrated. The crude material was purified on silica gel column chromatography (from PE/EtOAc=5/1 to 3/1, TLC: PE/EtOAc=3/1) to yield tert-butyl 4-(6-bromo-4-fluoro-1,3-benzoxazol-2-yl)piperidine-1-carboxylate (366 mg, 68% yield) as a yellow solid. [M-tBu+2+H]=344.8

Step c: To a solution of tert-butyl 4-(6-bromo-4-fluoro-1,3-benzoxazol-2-yl)piperidine-1-carboxylate (50 mg, 125.2 umol) and 2,8-dimethyl-6-(4,4,5,5-tetramethyl-1,3,2-dioxaborolan-2-yl)imidazo[1,2-b]pyridazine (34.2 mg, 125.2 μmol) in dioxane (3 mL) and water (1 mL) was added Pd(dppf)Cl2 (9.16 mg, 12.52 μmol) and K₂CO₃ (34.6 mg, 250.4 μmol). The mixture was degassed with N₂ for 3 times and it was stirred at 80° C. for 2 h. The reaction mixture was concentrated in vacuo to give the residue, which was purified by prep-HPLC (NH₃·H₂O modifier) to give tert-butyl 4-[6-(2,8-dimethylimidazo[1,2-b]pyridazin-6-yl)-4-fluoro-1,3-benzoxazol-2-yl]piperidine-1-carboxylate (30 mg, 51% yield) as a white solid. [M+H]=466.3 Step d: To a solution of tert-butyl 4-[6-(2,8-dimethylimidazo[1,2-b]pyridazin-6-yl)-4-fluoro-1,3-benzoxazol-2-yl]piperidine-1-carboxylate (20 mg, 42.9 μmol) in 1,1,1,3,3,3-hexafluoropropan-2-ol (1 mL) was added TFA (14.70 mg, 128.8 μmol, 9.87 μL). The mixture was stirred at 20° C. for 1 h. The reaction mixture was diluted with CH₃CN (2 mL) and it was purified by prep-HPLC (Column: Boston Green ODS 150*30 mm*5 um; Condition water (FA)-ACN, Begin B 42, End B 72; Gradient Time (min): 12; 100% B Hold Time (min): 2; Flow Rate (ml/min): 25) to give 6-(2,8-dimethylimidazo[1,2-b]pyridazin-6-yl)-4-fluoro-2-(4-piperidyl)-1,3-benzoxazole (11.2 mg, 71% yield) as a white solid. [M+H]=366.1; ¹H NMR (400 MHz, METHANOL-d4) δ: 8.16 (s, 1H), 7.96 (s, 1H), 7.88 (m, 1H), 7.66 (s, 1H), 3.80-3.75 (m, 1H), 3.20-3.10 (m, 2H), 2.80-2.70 (m, 2H), 2.69 (s, 3H), 2.51 (s, 3H), 2.25-2.15 (m, 2H), 2.20-2.10 (m, 2H).

Using the procedure described for Example 23 above, additional compounds described herein were prepared by substituting the appropriate boronic ester in step c, suitable reagents, and reaction conditions, obtaining compounds such as those selected from:

| Cpd ID | Data |
|---|---|
| 155 | Starting material: 2,8-dimethyl-6-(4,4,5,5-tetramethyl-1,3,2-dioxaborolan-2- |
| | yl)imidazo[1,2-a]pyridine |
| | MS: m/z 365.2 [M + H]⁺; ¹H NMR (400 MHz, METHANOL-d4) δ 8.42 (s, |
| | 1H), 7.59 (s, 1H), 7.50 (s, 1H), 7.35-7.24 (m, 2H), 3.16-3.05 (m, 3H), 2.77- |
| | 2.66 (m, 2H), 2.475 (s, 3H), 2.33 (s, 3H), 2.13-2.04 (m, 2H), 1.88-1.74 (m, |
| | 2H) |
| 156 | Starting material: 8-fluoro-2-methyl-6-(4,4,5,5-tetramethyl-1,3,2- |
| | dioxaborolan-2-yl)imidazo[1,2-a]pyridine |
| | MS: m/z = 365.2; 1H NMR (400 MHz, METHANOL-d4) δ 8.61 (s, 1H), |
| | 7.92-7.59 (m, 2H), 7.57-7.41 (m, 2H), 3.30-3.21 (m, 3H), 3.03-2.79 (m, 2H), |
| | 2.45 (s, 3H), 2.34-2.17 (m, 2H), 2.08-1.86 (m, 2H) |
| 157 | Starting material: 2,7-dimethyl-5-(4,4,5,5-tetramethyl-1,3,2-dioxaborolan-2- |
| | yl)-2H-indazole |
| | [M + H] = 365.2; ¹H NMR (400 MHz, METHANOL-d4) δ: 8.24 (s, 1H), 7.80 |
| | (s, 1H), 7.71 (s, 1H), 7.45-7.40 (m, 1H), 7.40 (s, 1H), 4.25 (s, 3H), 3.30-3.20 |
| | (m, 3H), 2.90-2.80 (m, 2H), 2.64 (s, 3H), 2.30-2.20 (m, 2H), 2.00-1.90 (m, |
| | 2H). |
| 455 | Starting material: (1R,5S,6r)-3-(tert-butoxycarbonyl)-3- |
| | azabicyclo[3.1.0]hexane-6-carboxylic acid |
| | MS: m/z 364.1 [M + H]⁺; RT: 1.630 min (Method 10) |
| 458 | Starting material: 7-(tert-butoxycarbonyl)-7-azaspiro[3.5]nonane-2- |
| | carboxylic acid |
| | MS: m/z 420.2 [M + H]⁺; RT: 1.71 min (Method 10) |
| 459 | Starting material: 2-(1-(tert-butoxycarbonyl)piperidin-4-yl)acetic acid |
| | MS: m/z 394.1 [M + H]⁺; RT: 1.735 min (Method 8) |
| 460 | Starting material: (1s,4s)-4-((tert-butoxycarbonyl)amino)cyclohexane-1- |
| | carboxylic acid |
| | MS: m/z 408.2 [M + H]⁺; RT: 1.681 min (Method 10) |
| 461 | Starting material: (1r,4r)-4-((tert-butoxycarbonyl)amino)cyclohexane-1- |
| | carboxylic acid |
| | MS: m/z 408.2 [M + H]⁺; RT: 1.656 min (Method 10) |
| 462 | Starting material: 8-(tert-butoxycarbonyl)-8-azaspiro[4.5]decane-2- |
| | carboxylic acid |
| | MS: m/z 434.2 [M + H]⁺; RT: 1.717 min (Method 10) |
| 463 | Starting material: 8-(tert-butoxycarbonyl)-8-azaspiro[4.5]decane-2- |
| | carboxylic acid |
| | MS: m/z 434.2 [M + H]⁺; RT: 1.737 min (Method 10) |
| 464 | Starting material: 3-((dimethylamino)methyl)cyclobutane-1-carboxylic acid |
| | MS: m/z 394.1 [M + H]⁺; RT: 1.785 min (Method 8) |
| 465 | Starting material: 3-((dimethylamino)methyl)cyclobutane-1-carboxylic acid |
| | MS: m/z 394.1 [M + H]⁺; RT: 1.795 min (Method 8) |

### Example 25—Compound 439

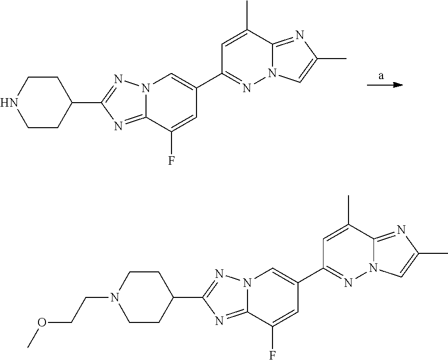

Step a: To a solution of 6-(2,8-dimethylimidazo[1,2-b]pyridazin-6-yl)-8-fluoro-2-(4-piperidyl)-[1,2,4]triazolo[1,5-a]pyridine (30 mg, 82 umol), 1-bromo-2-methoxy-ethane (11 mg, 82 umol, 8 uL) and Cs₂CO₃ (54 mg, 164 umol) in DMF (3 mL) at 25° C., the mixture was stirred at 90° C. for 1 hour. The mixture was concentrated in vacuo to give crude product. The crude was purified by prep. HPLC together (Boston Prime C18 150*30 mm*5 um, water (NH3H2O+NH4HCO3)-ACN as a mobile phase, from 29% to 59%, Gradient Time (min): 10, Flow Rate (ml/min): 25) to give 6-(2,8-dimethylimidazo[1,2-b]pyridazin-6-yl)-8-fluoro-2-[1-(2-methoxyethyl)-4-piperidyl]-[1,2,4]triazolo[1,5-a]pyridine (9.3 mg, 21.96 umol, 26.75% yield, 100% purity) as a white solid. MS: m/z 424.2 [M+H]⁺; RT: 1.612 min (Method 10)

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 436 | Starting material: 1-neopentylpiperidine-4-carbaldehyde |
| | MS: m/z 436.2 [M + H]⁺; RT: 1.416 min (Method 10) |
| 438 | Starting material: cyclopropanecarbaldehyde |
| | MS: m/z 420.1 [M + H]⁺; RT: 2.127 min (Method 10) |
| 441 | Starting material: pyrimidine-2-carbaldehyde |
| | MS: m/z 458.1 [M + H]⁺; RT: 1.293 min (Method 10) |
| 444 | Starting material: cyclobutanecarbaldehyde |
| | MS: m/z 434.2 [M + H]⁺; RT: 2.12 min (Method 10) |

### Example 26—Compound 443 & 453

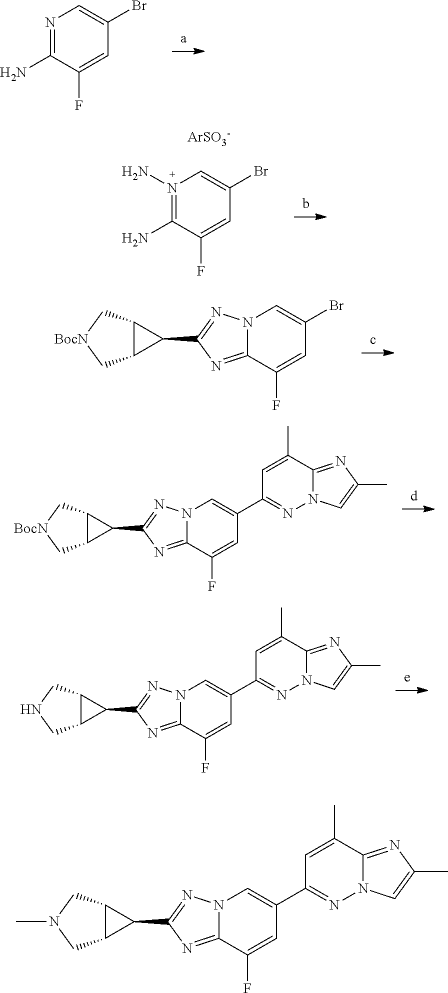

Step a: To a solution of 5-bromo-3-fluoro-pyridin-2-amine (2 g, 10 mmol) in MeCN (15 mL) was added O-(2,4-dinitrophenyl)hydroxylamine (2.1 g, 10 mmol) at 25° C. The mixture was stirred at 40° C. for 16 h. The mixture is filtered to remove the solvent to give 5-bromo-3-fluoro-pyridin-1-ium-1,2-diamine (3 g, crude) as a yellow solid. Then 5-bromo-3-fluoro-pyridin-1-ium-1,2-diamine (3 g, crude) was dissolved in DMSO (6 mL) to store. MS: m/z 206.0 [M+H]⁺; RT: 0.463 min (Method 10)

Step b: To a solution of 5-bromo-3-fluoro-pyridine-1,2-diamine (98 mg, 473 umol, 1.5 mL) and tert-butyl (1S,5R)-6-formyl-3-azabicyclo[3.1.0]hexane-3-carboxylate (100 mg, 473 umol) in MeOH (4 mL), was added KOH (2 M, 355 uL), the mixture was stirred at 25° C. for 1 h., then the reaction mixture was partitioned between EtOAc (30 mL) and water (5 mL). After quenching the reaction, the reaction mixture was poured into separatory funnel and separated. Then the mixture was dissolved in MeOH and filtered, which was purified with prep-HPLC (Column: Boston Green ODS 150*30 mm*5 um; Mobile Phase: from 40% to 80% of water (FA)-ACN to give tert-butyl (1S,5R)-6-(6-bromo-8-fluoro-[1,2,4]triazolo[1,5-a]pyridin-2-yl)-3-azabicyclo[3.1.0]hexane-3-carboxylate (97 mg, 243 umol, 51% yield) as white solid. MS: m/z 397.1 [M+H]⁺; RT: 1.147 min (Method 7)

Step c: To a solution of tert-butyl (1S,5R)-6-(6-bromo-8-fluoro-[1,2,4]triazolo[1,5-a]pyridin-2-yl)-3-azabicyclo[3.1.0]hexane-3-carboxylate (40 mg, 100 umol) and 2,8-dimethyl-6-(4,4,5,5-tetramethyl-1,3,2-dioxaborolan-2-yl)imidazo[1,2-b]pyridazine (33 mg, 120 umol) in dioxane (3 mL) and water (1 mL) was added Pd(dppf)Cl₂ (1.5 mg, 2 umol) and K₂CO₃ (28 mg, 201 umol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified with column on silica gel (40% to 100% EtOAc:PE) to give tert-butyl (1S,5R)-6-[6-(2,8-dimethylimidazo[1,2-b]pyridazin-6-yl)-8-fluoro-[1,2,4]triazolo[1,5-a]pyridin-2-yl]-3-azabicyclo[3.1.0]hexane-3-carboxylate (30 mg, 64 umol, 64% yield) as a yellow solid. MS: m/z 464.2 [M+H]⁺; RT: 1.999 min (Method 10)

Step d: To a solution of tert-butyl (1R,5S)-6-[6-(2,8-dimethylimidazo[1,2-b]pyridazin-6-yl)-8-fluoro-[1,2,4]triazolo[1,5-a]pyridin-2-yl]-3-azabicyclo[3.1.0]hexane-3-carboxylate (15 mg, 32 umol) in Hexafluoroisopropanol (4 mL) was added TFA (18 mg, 161 umol, 12 uL), the mixture was stirred at 25° C. for 1 h. Evaporate the solution on a water bath under reduced pressure, then the mixture was dissolved in MeOH and filtered, which was purified with prep-HPLC (Column: Boston Green ODS 150*30 mm*5 um; Mobile Phase: from 0% to 15% of water (FA)-ACN to give 2-[(1R,5S)-3-azabicyclo[3.1.0]hexan-6-yl]-6-(2,8-dimethylimidazo[1,2-b]pyridazin-6-yl)-8-fluoro-[1,2,4]triazolo[1,5-a]pyridine (10 mg, 28 umol, 86% yield) as white solid. MS: m/z 364.1 [M+H]⁺; RT: 1.213 min (Method 10) Step e: To a solution of 2-[(1S,5R)-3-azabicyclo[3.1.0]hexan-6-yl]-6-(2,8-dimethylimidazo[1,2-b]pyridazin-6-yl)-8-fluoro-[1,2,4]triazolo[1,5-a]pyridine (16 mg, 43 μmol) and paraformaldehyde (51 mg, 43 μmol, 58 μL) in MeOH (2 mL) was added Acetic acid (13 mg, 215 μmol, 12 μL) and the mixture was stirred at 25° C. for 20 min. Then sodium cyanoborohydride (8 mg, 129 μmol) was added and the mixture was stirred at 25° C. for 1 h. The mixture was filtered and the filtrate was concentrated in vacuo to give the residue, which was purified by prep-HPLC to give 6-(2,8-dimethylimidazo[1,2-b]pyridazin-6-yl)-8-fluoro-2-[(1S,5R)-3-methyl-3-azabicyclo[3.1.0]hexan-6-yl]-[1,2,4]triazolo[1,5-a]pyridine (9 mg, 25 μmol, 57% yield) as a yellow solid. MS: m/z 378.2 [M+H]⁺; RT: 1.233 min (Method 10)

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 440 | Starting material: tert-butyl 3-(2-oxoethyl)azetidine-1-carboxylate |
| | MS: m/z 352.1 [M + H]⁺; RT: 1.26. min (Method 10) |
| 442 | Starting material: tert-butyl 6-formyl-2-azaspiro[3.3]heptane-2-carboxylate |
| | MS: m/z 378.2 [M + H]⁺; RT: 1.69 min (Method 10) |
| 445 | Starting material: tert-butyl (5R)-5-formyl-2,2-dimethyl-piperidine-1-carboxylate |
| | MS: m/z 394.1 [M + H]⁺; RT: 1.35 min (Method 10) |
| 446 | Starting material: tert-butyl 2-(2-oxoethyl)morpholine-4-carboxylate |
| | MS: m/z 382.2 [M + H]⁺; RT: 1.12 min (Method 8) |
| 447 | Starting material: tert-butyl N-(4-formylcyclohexyl)carbamate |
| | MS: m/z 408.1 [M + H]⁺; RT: 2.53 min (Method 8) |
| 448 | Starting material: tert-butyl 4-(2-oxoethyl)piperidine-1-carboxylate |
| | MS: m/z 394.2 [M + H]⁺; RT: 1.41 min (Method 10) |
| 449 | Starting material: tert-butyl N-[3-(2-oxoethyl)cyclobutyl]carbamate |
| | MS: m/z 366.1 [M + H]⁺; RT: 1.58 min (Method 10) |
| 450 | Starting material: tert-butyl N-[3-(2-oxoethyl)cyclobutyl]carbamate |
| | MS: m/z 366.1 [M + H]⁺; RT: 1.57 min (Method 10) |
| 451 | Starting material: 5-bromo-3-fluoro-pyridin-1-ium-1,2-diamine |
| | MS: m/z 420.2 [M + H]⁺; RT: 1.35 min (Method 8) |
| 452 | Starting material: tert-butyl 8-formyl-2-azaspiro[4.5]decane-2-carboxylate |
| | MS: m/z 420.2 [M + H]⁺; RT: 1.32 min (Method 8) |
| 454 | Starting material: 5-bromo-3-fluoro-pyridin-1-ium-1,2-diamine |
| | MS: m/z 392.2 [M + H]⁺; RT: 1.67 min (Method 8) |
| 434 | Starting material: tert-butyl 4-formylpiperidine-1-carboxylate |
| | MS: m/z 380.1 [M + H]⁺; RT: 0.38 min (Method 4) |
| 433 | Starting material: tert-butyl 4-formylpiperidine-1-carboxylate |
| | MS: m/z 406.6 [M + H]⁺; RT: 0.41 min (Method 4) |
| 437 | Starting material: tert-butyl 4-formylpiperidine-1-carboxylate |
| | MS: m/z 420.2 [M + H]⁺; RT: 2.06 min (Method 8) |
| 435 | Starting material: tert-butyl 4-formylpiperidine-1-carboxylate |
| | MS: m/z 394.2 [M + H]⁺; RT: 1.73 min (Method 8) |
| 431 | Starting material: tert-butyl 4-formylpiperidine-1-carboxylate |
| | MS: m/z 365.1 [M + H]⁺; RT: 1.56 min (Method 8) |
| 432 | Starting material: tert-butyl 4-formylpiperidine-1-carboxylate |
| | MS: m/z 365.1 [M + H]⁺; RT: 1.45 min (Method 10) |

### Example 27—Compound 539

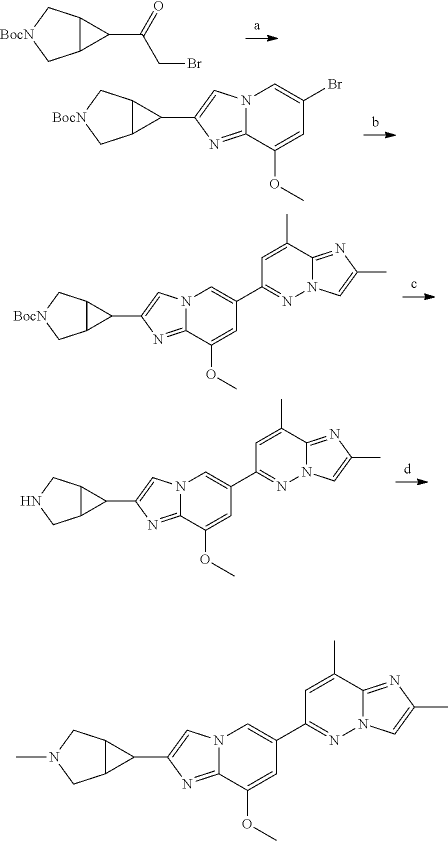

Step a: To a solution of tert-butyl (1S,5R)-6-(2-bromoacetyl)-3-azabicyclo[3.1.0]hexane-3-carboxylate (500 mg, 1.64 mmol) in t-BuOH (10 mL) was added NaHCO₃ (276 mg, 3.29 mmol, 127 uL) and 5-bromo-3-methoxy-pyridin-2-amine (333 mg, 1.64 mmol). The mixture was stirred at 80° C. for 12 h. The mixture was filtered and concentrated to give a residue. The residue was purified by flash silica gel chromatography (from PE/EtOAc=10/1 to 0/1, TLC: PE/EtOAc=1/1, Rf=0.45) to yield the product. Compound tert-butyl (1S,5R)-6-(6-bromo-8-methoxy-imidazo[1,2-a]pyridin-2-yl)-3-azabicyclo[3.1.0]hexane-3-carboxylate (323 mg, 791 umol, 48% yield) was obtained as yellow solid. MS: m/z 408.0 [M+H]⁺; RT: 0.375 min (Method 9)

Step b: To a stirred solution of tert-butyl (1S,5R)-6-(6-bromo-8-methoxy-imidazo[1,2-a]pyridin-2-yl)-3-azabicyclo[3.1.0]hexane-3-carboxylate (70 mg, 171 umol) and 2,8-dimethyl-6-(4,4,5,5-tetramethyl-1,3,2-dioxaborolan-2-yl)imidazo[1,2-b]pyridazine (47 mg, 171 umol) in Dioxane (5 mL) was added K₂CO₃ (71 mg, 514 umol) and Pd(dppf)Cl₂ (13 mg, 17 umol). The reaction mixture was stirred at 90° C. for 1 h. The mixture was filtered and concentrated to give a residue. The residue was purified by prep-HPLC purification (neutral condition). Compound tert-butyl (1S,5R)-6-[6-(2,8-dimethylimidazo[1,2-b]pyridazin-6-yl)-8-methoxy-imidazo[1,2-a]pyridin-2-yl]-3-azabicyclo[3.1.0]hexane-3-carboxylate (30 mg, 62 umol, 36% yield) was obtained as a white solid. MS: m/z 475.3 [M+H]⁺; RT: 0.283 min (Method 9)

Step c: To a solution of tert-butyl (1S,5R)-6-[6-[8-(difluoromethyl)-2-methyl-imidazo[1,2-b]pyridazin-6-yl]-8-methoxy-imidazo[1,2-a]pyridin-2-yl]-3-azabicyclo[3.1.0]hexane-3-carboxylate (12 mg, 23 umol) in HCl/EtOAc (2 mL). The mixture was stirred at 25° C. for 8h. The mixture was filtered and concentrated to give a residue. The crude compound was used into the next step without further purification. Compound 6-[2-[(1S,5R)-3-azabicyclo[3.1.0]hexan-6-yl]-8-methoxy-imidazo[1,2-a]pyridin-6-yl]-8-(difluoromethyl)-2-methyl-imidazo[1,2-b]pyridazine (8 mg, 19.5 umol, 82% yield) was obtained as white solid. MS: m/z 411.1 [M+H]⁺; RT: 0.241 min (Method 9)

Step d: To a stirred solution of 6-[2-[(1 S,5R)-3-azabicyclo[3.1.0]hexan-6-yl]-8-methoxy-imidazo[1,2-a]pyridin-6-yl]-2,8-dimethyl-imidazo[1,2-b]pyridazine (20 mg, 53 umol) and paraformaldehyde (64 mg, 53 umol, 73 uL) in DCE/EtOH (6 mL) was added TEA (16 mg, 160 umol, 23 uL) and stirred at 20° C. for 15 min. After addition, the mixture was added sodium triacetoxyborohydride (23 mg, 107 umol) and stirred at 20° C. for 2 h. The mixture was filtered and concentrated to give a residue. The residue was purified by prep-HPLC purification (neutral condition). Compound 6-[8-methoxy-2-[(1S,5R)-3-methyl-3-azabicyclo[3.1.0]hexan-6-yl]imidazo[1,2-a]pyridin-6-yl]-2,8-dimethyl-imidazo[1,2-b]pyridazine (8 mg, 20 umol, 37% yield) was obtained as a yellow solid. MS: m/z 389.1 [M+H]⁺; RT: 0.183 min (Method 9)

Using the procedure described for Example 1 above, additional compounds described herein were prepared by substituting the appropriate bromoketone starting material in step a, the appropriate boronic ester or acid equivalent in step b, suitable reagents and reaction conditions, obtaining compounds such as those selected from:

| Cpd ID | Data |
|---|---|
| 538 | Starting material: tert-butyl 4-(2-bromoacetyl)piperidine-1-carboxylate |
| | MS: m/z 377.3 [M + H]⁺; RT: 0.72 min (Method 3) |
| | ¹H NMR (600 MHz, DMSO-d₆) δ ppm 1.84-1.92 (m, 2 H) 2.18 (br d, |
| | J = 12.21 Hz, 2 H) 2.45 (s, 3 H) 2.65 (d, J = 0.76 Hz, 3 H) 3.04-3.13 (m, 3 H) |
| | 3.39 (br d, J = 12.59 Hz, 2 H) 4.09 (s, 3 H) 7.40 (br d, J = 8.39 Hz, 1 H) 7.76- |
| | 7.82 (m, 1 H) 7.94 (br s, 1 H) 8.38 (br d, J = 9.54 Hz, 1 H) 8.65 (br d, J = 9.16 |
| | Hz, 1 H) 8.99 (s, 1 H) |

### Example 28—Compound 537

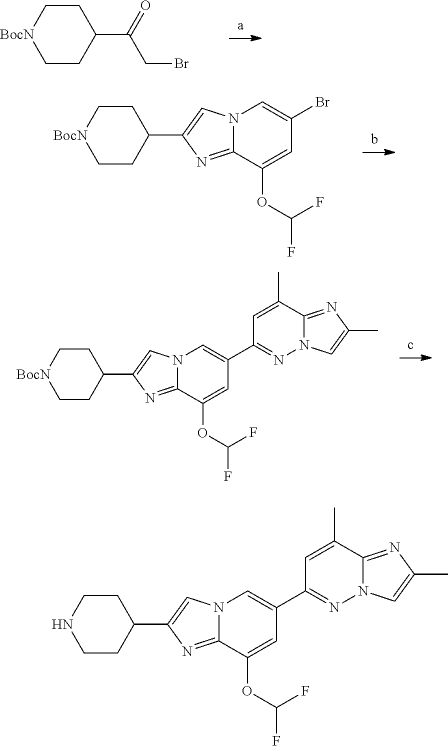

Step a: tert-butyl 4-(2-bromoacetyl)piperidine-1-carboxylate (400.33 mg, 1.31 mmol, 1.25 eq.) and 5-bromo-3-(difluoromethoxy)pyridin-2-amine (250 mg, 1.05 mmol, 1.0 eq.) were dissolved in Water (5.23 mL) which was heated to 80° C. for 16 hours before being concentrated and dry loaded onto normal phase silica and purified 0-25% MeOH:DCM over 12 minutes. Deboc material elutes at 20% MeOH, boc material elutes at 4% MeOH. Identified fractions were collected, concentrated, quantified, and carried forward as is. MS: m/z 392.1 [M+H-tBu]⁺; RT 0.67 min (Method 4).

Step b: tert-butyl 4-[6-bromo-8-(difluoromethoxy)imidazo[1,2-a]pyridin-2-yl]piperidine-1-carboxylate (98.7 mg, 221 umol, 1.0 eq.) was dissolved in Dioxane (1 mL, 0.2 M) and Water (1 mL, 0.2 mL) before 2,8-dimethyl-6-(4,4,5,5-tetramethyl-1,3,2-dioxaborolan-2-yl)imidazo[1,2-b]pyridazine (66.4 mg, 239 umol, 1.1 eq.) and Cesium carbonate (216.2 mg, 663 umol, 3 eq.) were added. The solution was then thoroughly degassed with nitrogen before Pd(dppf)Cl2 DCM (18 mg, 22.1 umol, 0.1 eq.) was added. The solution was then heated at 80° C. for 2 hours telescoped forward crude assuming 100% yield.

Step c: tert-butyl 4-[8-(difluoromethoxy)-6-(2,8-dimethylimidazo[1,2-b]pyridazin-6-yl)imidazo[1,2-a]pyridin-2-yl]piperidine-1-carboxylate (113.4 mg, 221.2 umol, 1.0 eq.) was dissolved in DCM (3 mL, 0.07 M) before 4 M HCl in dioxane (80.63 mg, 2.21 mmol, 10.0 eq.) was added. The solution then stirred at 50° C. for 2 hours before being concentrated then taken back up in DMSO, filtered and purified via HPLC purification (Column: Sunfire C18 100×19 mm, 5 mm; Mobile phase A: MeCN; Mobile phase B: H2O; Modifier: 0.1% TFA). Identified fractions were collected, combined, and concentrated to obtain 6-[8-(difluoromethoxy)-2-(4-piperidyl)imidazo[1,2-a]pyridin-6-yl]-2,8-dimethyl-imidazo[1,2-b]pyridazine as a yellow oil (18.3 mg, 15.7% yield). MS: m/z 413.3 [M+H]⁺; RT 1.88 min (Method 1). 1H NMR (600 MHz, DMSO-d6) δ ppm 1.87-1.97 (m, 2H) 2.15-2.19 (m, 2H) 2.19-2.49 (m, 3H) 2.52-2.73 (m, 3H) 3.05-3.20 (m, 4H) 3.43-3.57 (m, 1H) 7.66-7.68 (m, 1H) 7.69-7.71 (m, 1H) 7.72-7.75 (m, 1H) 7.95-7.98 (m, 1H) 8.12-8.16 (m, 1H) 8.27-8.36 (m, 1H) 8.54-8.65 (m, 1H) 9.19-9.23 (m, 1H)

Using the procedure described for Example 1 above, additional compounds described herein were prepared by substituting the appropriate bromoketone starting material in step a, the appropriate boronic ester or acid equivalent in step b, suitable reagents and reaction conditions, obtaining compounds such as those selected from:

| Cpd ID | Data |
|---|---|
| 536 | Starting material: 2,8-dimethyl-6-(4,4,5,5-tetramethyl-1,3,2-dioxaborolan-2- |
| | yl)imidazo[1,2-b]pyridazine |
| | MS: m/z 416.3 [M + H]⁺; RT: 1.76 min (Method 1) |
| | 1H NMR (600 MHz, DMSO-d6) δ ppm 1.57-1.68 (m, 2 H) 1.88-1.99 (m, |
| | 2 H) 2.26-2.54 (m, 3 H) 2.62 (m, 2 H) 2.80-2.89 (m, 1 H) 2.99-3.18 (m, |
| | 2 H) 7.39-7.45 (m, 1 H) 7.51-7.58 (m, 1 H) 7.61-7.76 (m, 1 H) 7.78-7.81 |
| | (m, 1 H) 7.83-7.86 (m, 1 H) 8.79-8.85 (m, 2 H) |

### Example 29—Compound 549

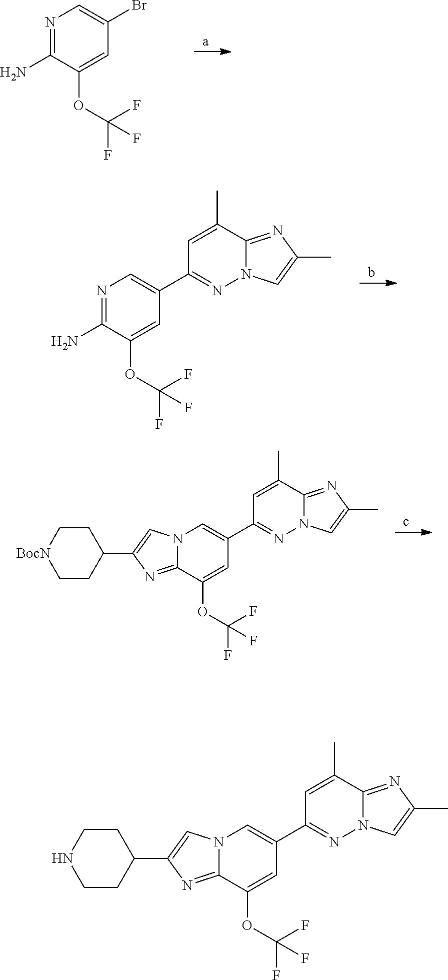 [SMILES: Nc1ncc(Br)cc1OC(F)(F)F ; Cc1cn2nc(-c3cnc(N)c(OC(F)(F)F)c3)cc(C)c2n1 ; Cc1cn2nc(-c3cc(OC(F)(F)F)c4nc(C5CCN(C(=O)OC(C)(C)C)CC5)cn4c3)cc(C)c2n1 ; Cc1cn2nc(-c3cc(OC(F)(F)F)c4nc(C5CCNCC5)cn4c3)cc(C)c2n1]

Step a: To a solution of 5-bromo-3-(trifluoromethoxy)pyridin-2-amine (250 mg, 0.97 mmol) and (2,8-dimethylimidazo[1,2-b]pyridazin-6-yl)boronic acid (265.7 mg, 0.97 mmol) in dioxane (4.6 mL) and water (1.6 mL) was added Cs₂CO₃ (950.8 mg, 2.92 mmol) and Pd(dppf)Cl₂ (79.4 mg, 97.2 μmol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified by flash silica gel chromatography (0% to 20% MeOH:DCM) to give 5-(2,8-dimethylimidazo[1,2-b]pyridazin-6-yl)-3-(trifluoromethoxy)pyridin-2-amine (247.2 mg, 0.76 mmol, 79% yield) as a yellow solid. MS: m/z 324.1 [M+H]⁺.

Step b: To a solution of tert-butyl 4-(2-bromoacetyl)piperidine-1-carboxylate (56.8 mg, 185.6 μmol) in t-BuOH (2 mL) was added 5-(2,8-dimethylimidazo[1,2-b]pyridazin-6-yl)-3-(trifluoromethoxy)pyridin-2-amine (60 mg, 185.6 μmol) and NaHCO₃ (46.8 mg, 556.8 μmol) and the mixture was stirred at 80° C. for 16 h. The mixture was concentrated in vacuo to give the residue, which was purified by flash silica gel chromatography (0% to 10% MeOH:DCM) to give tert-butyl 4-(6-(2,8-dimethylimidazo[1,2-b]pyridazin-6-yl)-8-(trifluoromethoxy)imidazo[1,2-a]pyridin-2-yl)piperidine-1-carboxylate (69.2 mg, 70% yield) as a yellow solid. MS: m/z 531.3 [M+H]⁺.

Step c: 4-(6-(2,8-dimethylimidazo[1,2-b]pyridazin-6-yl)-8-(trifluoromethoxy)imidazo[1,2-a]pyridin-2-yl)piperidine-1-carboxylate (69.2 mg, 130.4 μmol) was dissolved in 4 M HCl in dioxane (0.26 mL) and DCM (2 mL). The mixture was stirred at 25° C. for 2 h. The mixture was concentrated in vacuo and purified by preparative HPLC (Column: Sunfire C18 100×19 mm, 5 mm; Mobile phase A: MeCN; Mobile phase B: H₂O; Modifier: 0.1% TFA) to give 2,8-dimethyl-6-(2-(piperidin-4-yl)-8-(trifluoromethoxy)imidazo[1,2-a]pyridin-6-yl)imidazo[1,2-b]pyridazine trifluoroacetic acid slat (1.3 mg, 1.8% yield) as a white solid. MS: m/z 431.3 [M+H]⁺.

### Example 30—Compound 551 & 552

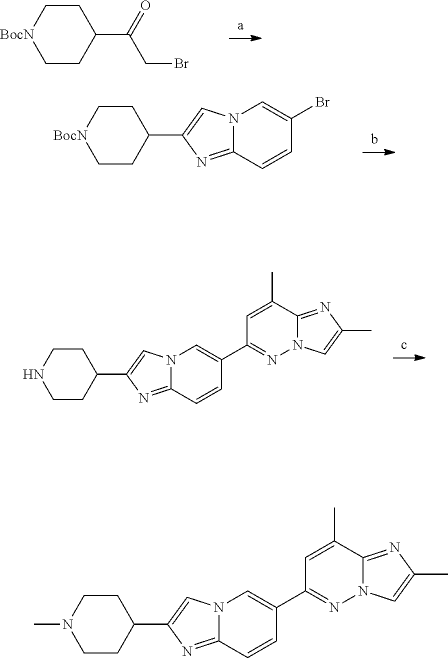

Step a: 5-bromopyridin-2-amine (500 mg, 2.89 mmol) was dissolved in ACN (3.47 mL) and toluene (2.31 mL). Sodium bicarbonate (242 mg, 2.89 mmol) was added and tert-butyl 4-(2-bromoacetyl)piperidine-1-carboxylate (884 mg, 2.89 mmol) followed. The mixture was stirred at 90° C. over night. The resulting was concentrated and purified by flash with a gradient of 0-100% EtOAc/heptane. MS: m/z 415.2 [M+H]⁺; RT: 0.86 min (Method 4)

Step b: tert-butyl 4-(6-bromoimidazo[1,2-a]pyridin-2-yl)piperidine-1-carboxylate (200 mg, 525.93 umol) and 2,8-dimethyl-6-(4,4,5,5-tetramethyl-1,3,2-dioxaborolan-2-yl)imidazo[1,2-b]pyridazine (158 mg, 578 umol) were mixed in Dioxane (1.58 mL) and water (1 mL) with PdCl2(dippf) (31 mg, 52 umol) and Cesium carbonate (514 mg, 1.6 mmol). The mixture was purged with N₂ and then warmed in the microwave for 1 h at 90° C. The solution was diluted with water, extracted with EtOAc and dried over sodium sulfate. The resulting was taken in DCM 2 mL and treated with 2 ml of HCl 4N/Dioxane. The solution was then stirred at room temp for an additional 3 h. The solution was concentrated and purified by acidic prep-HPLC. MS: m/z 347.1 [M+H]⁺; RT 0.54 min (Method 3).

Step c: 2,8-dimethyl-6-[2-(4-piperidyl)imidazo[1,2-a]pyridin-6-yl]imidazo[1,2-b]pyridazine (10 mg, 28.9 umol) was dissolved in ACN (1 mL) paraformaldehyde (35 mg, 28.9 umol, 40 uL), Acetic acid (17 mg, 289 umol, 17 uL), Sodium cyanoborohydride (6 mg, 87 umol) and warmed to 70° C. over night. the resulting was concentrated and purified by acidic prep-HPLC. MS: m/z 361.1 [M+H]⁺; RT 1.032 min (Method 10).

Using the procedure described for Example 1 above, additional compounds described herein were prepared by substituting the appropriate bromoketone starting material in step a, the appropriate boronic ester or acid equivalent in step b, suitable reagents and reaction conditions, obtaining compounds such as those selected from:

| Cpd ID | Data |
|---|---|
| 553 | Starting material: 2-bromo-1-((1R,5S,6r)-3-methyl-3- |
| | azabicyclo[3.1.0]hexan-6-yl)ethan-1-one |
| | MS: m/z 390.1 [M + H]⁺; RT: 0.252 min (Method 9) |
| 554 | Starting material: 2-bromo-1-((1R,5S,6r)-3-methyl-3- |
| | azabicyclo[3.1.0]hexan-6-yl)ethan-1-one |
| | MS: m/z 359.0 [M + H]⁺; RT: 0.651 min (Method 10) |

### Example 31—Compound 557

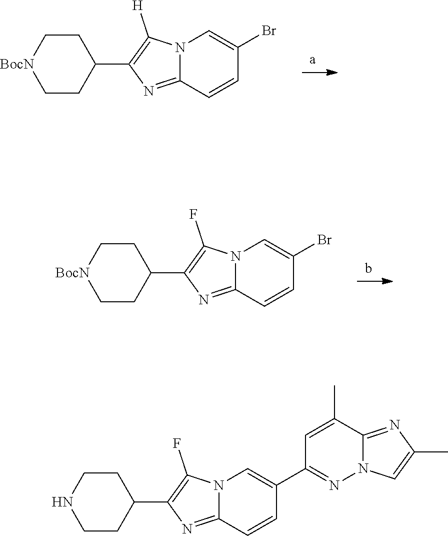 [SMILES: CC(C)(C)OC(=O)N1CCC(c2cn3cc(Br)ccc3n2)CC1 ; CC(C)(C)OC(=O)N1CCC(c2nc3ccc(Br)cn3c2F)CC1 ; Cc1cn2nc(-c3ccc4nc(C5CCNCC5)c(F)n4c3)cc(C)c2n1]

Step a: tert-butyl 4-(6-bromoimidazo[1,2-a]pyridin-2-yl)piperidine-1-carboxylate (270 mg, 710 umol) was dissolved in Chloroform (4 mL) at 0° C. DMAP (87 mg, 710 umol) was then added followed by 1-(chloromethyl)-4-fluoro-1,4-diazoniabicyclo[2.2.2]octane; ditetrafluoroborate (503 mg, 1.42 mmol). The reaction was stirred at 0 for 2 h, then the reaction was heated to 55° C. for 6h, quenched with a saturated aqueous solution of Sodium carbonate and extracted with EtOAc. The crude was purified by RPHPLC using an acidic gradient of 0-70% ACN-water. MS: m/z 397.0 [M+H]⁺; RT: 0.70 min (Method 4).

Step b: Tert-butyl 4-(6-bromo-3-fluoro-imidazo[1,2-a]pyridin-2-yl)piperidine-1l-carboxylate (45 mg, 113 umol) and 2,8-dimethyl-6-(4,4,5,5-tetramethyl-1,3,2-dioxaborolan-2-yl)imidazo[1,2-b]pyridazine (34 mg, 124 umol) were dissolved in Dioxane (1.6 mL) water (1.1 mL) with PdCl₂(dippf) (7 mg, 11.3 umol) and Cesium carbonate (110 mg, 339 umol). This was degassed with N₂ and microwaved at 90° C. for 1 h. The resulting was extracted with water/EtOAc and dried over sodium sulfate. The organics were concentrated and dissolved in DCM (2 mL) and treated with HCl 4N dioxane (2 mL). The solution was stirred at room temp for 3 h and then concentrated and purified by acidic prep-HPLC. MS: m/z 365.1 [M+H]⁺; RT: 0.68 min (Method 3).

### Example 32—Compound 350 & 363

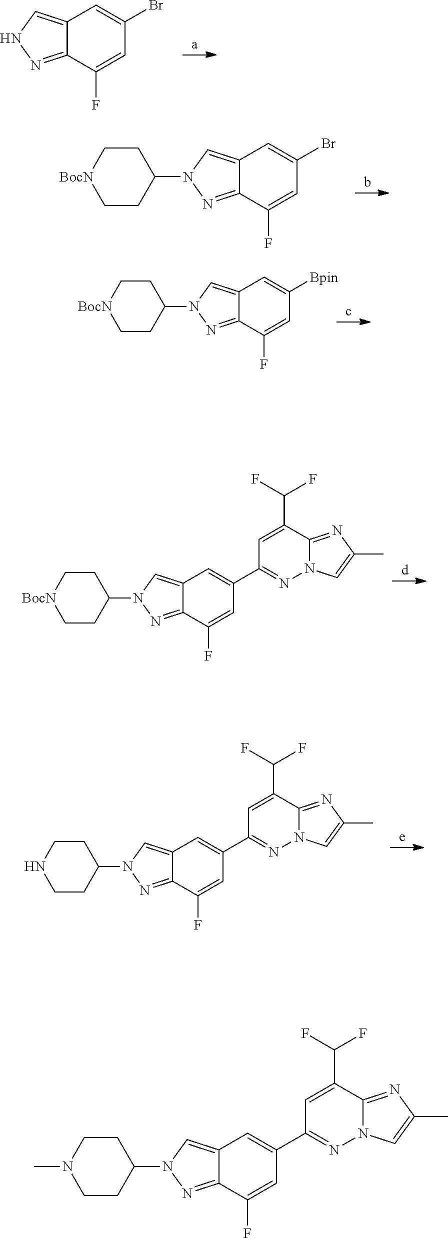

Step a: To a solution of tert-butyl 4-(p-tolylsulfonyloxy)piperidine-1-carboxylate (1.36 g, 3.83 mmol) in DMF (10 mL) was added K₂CO₃ (1.59 g, 11.49 mmol) and 5-bromo-7-fluoro-2H-indazole (823 mg, 3.83 mmol). The mixture was stirred at 100° C. for 12 h. The reaction mixture was diluted with H₂O (40 mL) and extracted with EtOAc (10 mL×3). The combined organic layers were washed with brine (30 mL×2), dried over Na₂SO₄, filtered and concentrated under reduced pressure to give a residue. The residue was purified by flash silica gel chromatography (from PE/EtOAc=10/1 to 5/1, TLC: PE/EtOAc=5/1, Rf=0.30) to yield the product. Compound tert-butyl 4-(5-bromo-7-fluoro-indazol-2-yl)piperidine-1-carboxylate (492 mg, 1.24 mmol, 32% yield) was obtained as yellow oil. MS: m/z 342.0 [M−56]⁺; RT: 0.518 min (Method 9).

Step b: To a solution of tert-butyl 4-(5-bromo-7-fluoro-indazol-2-yl)piperidine-1-carboxylate (60 mg, 150 umol) in Dioxane (2 mL) was added KOAc (29 mg, 301 umol), 4,4,5,5-tetramethyl-2-(4,4,5,5-tetramethyl-1,3,2-dioxaborolan-2-yl)-1,3,2-dioxaborolane (46 mg, 181 umol) and PdCl2(dppf) (22 mg, 30 umol). The mixture was stirred at 90° C. for 2 h under N₂. The crude compound was used into the next step without further purification. MS: m/z 446.3 [M+H]⁺; RT: 0.813 min (Method 7).

Step c: To a solution of tert-butyl 4-[7-fluoro-5-(4,4,5,5-tetramethyl-1,3,2-dioxaborolan-2-yl)indazol-2-yl]piperidine-1-carboxylate (100 mg, 224 μmol) in Dioxane (2 mL) and H2O (1 mL) was added Cs₂CO₃ (219.5 mg, 673 μmol), 6-chloro-8-(difluoromethyl)-2-methyl-imidazo[1,2-b]pyridazine (32 mg, 134 μmol) and PdCl2(dppf) (10 mg, 13 μmol). The mixture was stirred at 90° C. for 2 h under N₂. The mixture was filtered and concentrated to give a residue. The residue was purified by column chromatography (SiO₂, Petroleum ether/Ethyl acetate=20:1 to 1:1). The desired product was obtained as white solid. MS: m/z 501.0 [M+H]⁺; RT: 0.80 min (Method 4).

Step d: To a solution of tert-butyl 4-(5-(8-(difluoromethyl)-2-methylimidazo[1,2-b]pyridazin-6-yl)-7-fluoro-2H-indazol-2-yl)piperidine-1-carboxylate (70 mg, 139.86 μmol) in DCM (2 ml) was added 4N HCl in dioxane (34 uL). The mixture was stirred at 25° C. for 1 h. The mixture was concentrated to give a residue. The crude compound was used into the next step without further purification. MS: m/z 400.9 [M+H]⁺; RT: 0.0.55 min (Method 4).

Step e: 8-(difluoromethyl)-6-[7-fluoro-2-(4-piperidyl)indazol-5-yl]-2-methyl-imidazo[1,2-b]pyridazine (43 mg, 109 umol) was dissolved in THF (1 mL) with paraformaldehyde (132 mg, 109 umol, 149 uL), Acetic acid (66 mg, 1.10 mmol, 63 uL), Sodium cyanoborohydride (21 mg, 329 umol). The mixture was warmed to 70° C. and stirred overnight. The resulting was diluted with water, extracted with EtOAc and concentrated. The crude was purified by FCC using a gradient of 0-100% EtOAc Heptane to afford the compound. MS: m/z 415.2 [M+H]⁺; RT: 1.83 min (Method 4).

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 357 | Starting material: 6-chloro-8-ethyl-2-methylimidazo[1,2-b]pyridazine |
| | MS: m/z 393.3 [M + H]⁺; RT: 1.0 min (Method 3) |
| 358 | Starting material: 6-chloro-8-methoxy-2-methyl-[1,2,4]triazolo[1,5- |
| | b]pyridazine |
| | MS: m/z 396.1 [M + H]⁺; RT: 0.878 min (Method 10) |
| 359 | Starting material: 6-chloro-2,8-dimethyl-[1,2,4]triazolo[1,5-b]pyridazine |
| | MS: m/z 380.1 [M + H]⁺; RT: 1.691 min (Method 8) |
| | 1H NMR (400 MHz, METHANOL-d4) δ ppm = 8.57 (d, J = 2.4 Hz, 1H), |
| | 8.30 (s, 1H), 8.04 (d, J = 1.2 Hz, 1H), 7.83-7.74 (m, 1H), 4.61-4.55 (m, 1H), |
| | 3.13-3.05 (m, 2H), 2.72 (s, 3H), 2.62 (s, 3H), 2.38 (s, 3H), 2.36-2.26 (m, 6H). |
| 561 | Starting material: 6-chloro-2-methyl-[1,2,4]triazolo[1,5-b]pyridazine |
| | MS: m/z 352.3 [M + H]⁺; RT: 1.19 min (Method 3) |
| 365 | Starting material: 6-chloro-2-methylimidazo[1,2-b]pyridazine |
| | MS: m/z 351.0 [M + H]⁺; RT: 0.4 min (Method 4) |
| 354 | Starting material: 6-chloro-8-(difluoromethyl)-2-methylimidazo[1,2- |
| | b]pyridazine |
| | MS: m/z 441.1 [M + H]⁺; RT: 1.21 min (Method 3) |
| 370 | Starting material: 2-chloro-4,6-dimethylpyrazolo[1,5-a]pyrazine |
| | MS: m/z 365.3 [M + H]⁺; RT: 0.65 min (Method 4) |
| 371 | Starting material: 2-chloro-4,6-dimethylpyrazolo[1,5-a]pyrazine |
| | MS: m/z 379.4 [M + H]⁺; RT: 0.46 min (Method 4) |
| 372 | Starting material: 2-chloro-4,6-dimethylpyrazolo[1,5-a]pyrazine |
| | MS: m/z 419.5 [M + H]⁺; RT: 0.5 min (Method 4) |
| 373 | Starting material: 6-chloro-2-methyl-8-(trifluoromethyl)imidazo[1,2- |
| | b]pyridazine |
| | MS: m/z 477.2 [M + H]⁺; RT: 1.662 min (Method 10) |
| | NMR: 1H NMR: (400 MHz, METHANOL-d4)) δ ppm = 8.58 (d, J = 2.4 |
| | Hz, 1H), 8.33 (s, 1H), 8.13 (d, J = 11.6 Hz, 2H), 7.83 (d, J = 12.8 Hz, |
| | 1H), 4.60-4.55 (m, 1H), 3.62-3.58 (m, 2H), 3.38 (s, 3H), 3.20 (d, J = 12.0 Hz, |
| | 2H), 2.72-2.68 (m, 2H), 2.54 (s, 3H), 2.33-2.29 (m, 2H), 2.28-2.26 (m, 4H). |

### Example 33—Compound 196

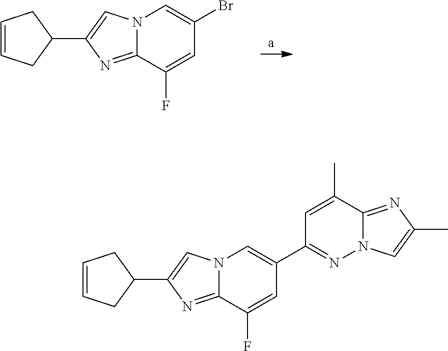 [SMILES: Fc1cc(Br)cn2cc(C3CC=CC3)nc12 ; Cc1cn2nc(-c3cc(F)c4nc(C5CC=CC5)cn4c3)cc(C)c2n1]

Step a: To a solution of 6-bromo-2-cyclopent-3-en-1-yl-8-fluoro-imidazo[1,2-a]pyridine (30 mg, 106 umol) in Dioxane (5 mL) was added 2,8-dimethyl-6-(4,4,5,5-tetramethyl-1,3,2-dioxaborolan-2-yl)imidazo[1,2-b]pyridazine (35 mg, 128 umol), Pd(dppf)Cl2 (8 mg, 11 umol) and K₂CO₃ (30 mg, 213 umol) under N₂. The mixture was stirred at 90° C. for 2 h. The reaction mixture was concentrated and then water (80 mL) was added. The mixture was extracted with EtOAc (50 mL×3). The combined organic layers were washed with brine (50 mL), dried over Na₂SO₄, filtered and concentrated in vacuo to give the residue, which was purified by prep-HPLC (FA) to give 6-(2-cyclopent-3-en-1-yl-8-fluoro-imidazo[1,2-a]pyridin-6-yl)-2,8-dimethyl-imidazo[1,2-b]pyridazine (6.6 mg, 19.00 umol, 17.80% yield) as yellow solid. MS: m/z 348.1 [M+H]⁺; RT: 1.88 min (Method 10)

### Example 34—Compound 226

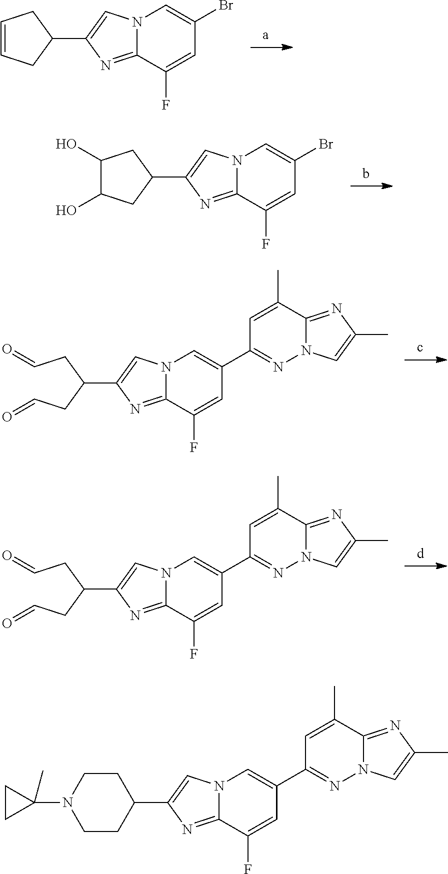

Step a: To a solution of 6-bromo-2-cyclopent-3-en-1-yl-8-fluoro-imidazo[1,2-a]pyridine (200 mg, 711 μmol) in Acetone (15 mL) and water (15 mL) was added NMO (166 mg, 1.42 mmol) dipotassium; dioxido(dioxo)osmium; dihydrate (52 mg, 142 μmol) at 25° C. The reaction was stirred at 25° C. for 16 hours. The reaction mixture was concentrated in vacuum to give crude product 4-(6-bromo-8-fluoro-imidazo[1,2-a]pyridin-2-yl)cyclopentane-1,2-diol (200 mg, 634 μmol, 89% yield) as a grey solid. MS: m/z 316.9 [M+2H]⁺; RT: 1.022 min (Method 10)

Step b: To a solution of 4-(6-bromo-8-fluoro-imidazo[1,2-a]pyridin-2-yl)cyclopentane-1,2-diol (200 mg, 634.64 μmol) in dioxane (10 mL) and water (10 mL) was added sodium; periodate (271 mg, 1.27 mmol) at 25° C. The reaction was stirred at 25° C. for 16 hours. The reaction mixture was concentrated in vacuum to give crude product. The crude product was purified by silica gel column chromatography (from PE/EtOAc=5/1 to 2/1) to give 3-(6-bromo-8-fluoro-imidazo[1,2-a]pyridin-2-yl)pentanedial (180 mg, 574.86 μmol, 90% yield) as a yellow solid. MS: m/z 314.9 [M+2H]⁺; RT: 1.035 min (Method 10)

Step c: To a mixture of 3-(6-bromo-8-fluoro-imidazo[1,2-a]pyridin-2-yl)pentanedial (80 mg, 255.49 μmol) and 1-methylcyclopropanamine (22 mg, 306.59 μmol) in EtOH (5 mL) DCE (5 mL) was added acetic acid (46 mg, 766 μmol, 44 μL) in one portion at 25° C. After 30 min, sodium; triacetoxyborohydride (162 mg, 766 μmol) was added. The mixture was concentrated and then water (60 mL) was added. The mixture was extracted with DCM (50 mL×3). The combined organic layers were dried over Na₂SO₄, filtered and concentrated. The crude material was purified by prep-HPLC (Column Phenomenex Gemini-NX 150*30 mm*5 um; Condition water (NH₄HCO₃)-ACN; Begin B 40; End B 70; Gradient Time (min) 11; 100% B Hold Time (min) 2; FlowRate (ml/min) 25) to yield 6-bromo-8-fluoro-2-[1-(1-methylcyclopropyl)-4-piperidyl]imidazo[1,2-a]pyridine (20 mg, 56.78 μmol, 22% yield) as a yellow solid. MS: m/z 352.1 [M+H]⁺; RT: 0.687 min (Method 10)

Step d: To a mixture of 6-bromo-8-fluoro-2-[1-(1-methylcyclopropyl)-4-piperidyl]imidazo[1,2-a]pyridine (20 mg, 56.78 μmol) and 2,8-dimethyl-6-(4,4,5,5-tetramethyl-1,3,2-dioxaborolan-2-yl)imidazo[1,2-b]pyridazine (17 mg, 62.46 μmol) in Dioxane (5 mL) was added Pd(dppf)Cl₂ (5 mg, 5.68 μmol) K₂CO₃ (24 mg, 170.34 μmol) in one portion at 25° C. under N₂. The mixture was stirred at 90° C. for 16 hours. The mixture was concentrated and then water was added. The mixture was extracted with EtOAc (30 mL×3). The combined organic layers were washed with brine, dried over Na₂SO₄, filtered, and concentrated. The crude material was purified by prep-HPLC(Column Phenomenex Gemini-NX 150*30 mm*5 um; Condition water (NH4HCO₃)-ACN; Begin B 40; End B 70; Gradient Time (min) 11; 100% B Hold Time (min) 2; FlowRate (ml/min) 25) to yield 6-[8-fluoro-2-[1-(1-methylcyclopropyl)-4-piperidyl]imidazo[1,2-a]pyridin-6-yl]-2,8-dimethyl-imidazo[1,2-b]pyridazine (3.7 mg, 8.84 μmol, 15% yield) as a yellow solid. MS: m/z 419.3 [M+H]⁺; RT: 1.935 min (Method 10)

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 205 | Starting material: |
| | MS: m/z 435.1 [M + H]⁺ ]⁺; RT: 1.698 min (Method 10) |

### Example 35—Compound 207

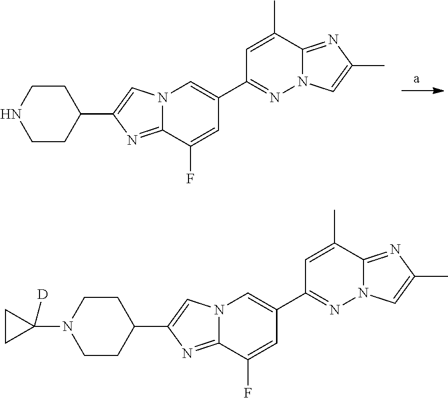 [SMILES: Cc1cn2nc(-c3cc(F)c4nc(C5CCNCC5)cn4c3)cc(C)c2n1 ; [2H]C1(N2CCC(c3cn4cc(-c5cc(C)c6nc(C)cn6n5)cc(F)c4n3)CC2)CC1]

Step a: 6-[8-fluoro-2-(4-piperidyl)imidazo[1,2-a]pyridin-6-yl]-2,8-dimethyl-imidazo[1,2-b]pyridazine (30 mg, 82 umol) and (1-ethoxycyclopropoxy)-trimethyl-silane (43 mg, 246 umol, 49.65 uL) were dissolved in THF (2 mL) and treated with deuterio 2,2,2-trideuterioacetate (52 mg, 823 umol, 50 uL) and Sodium cyanoborodeuteride (16 mg, 246.97 umol). the mixture was then stirred at 65° C. over night. The reaction was purified by prep-HPLC (TFA). MS: m/z 406.3 [M+H]⁺; RT: 0.86 min (Method 3)

### Example 36—Compound 555

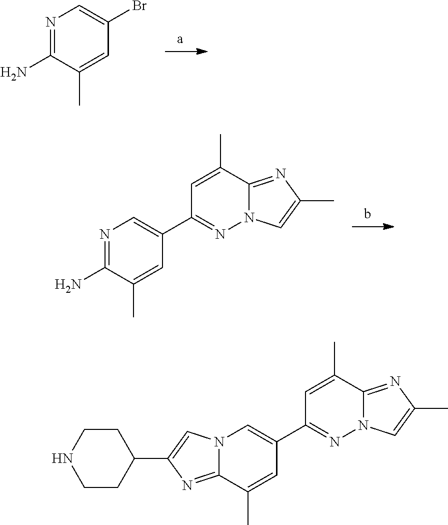

Step a: To a solution of 5-bromo-3-methylpyridin-2-amine (250 mg, 1.34 mmol) and (2,8-dimethylimidazo[1,2-b]pyridazin-6-yl)boronic acid (365.1 mg, 1.34 mmol) in dioxane (4.6 mL) and water (1.6 mL) was added Cs₂CO₃ (1.31 g, 4.01 mmol) and Pd(dppf)Cl₂ (109.2 mg, 133.7 μmol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified by flash silica gel chromatography (0% to 20% MeOH:DCM) to give 5-(2,8-dimethylimidazo[1,2-b]pyridazin-6-yl)-3-methylpyridin-2-amine (263.7 mg, 1.04 mmol, 78% yield) as a yellow solid. MS: m/z 254.1 [M+H]⁺.

Step b: To a solution of tert-butyl 4-(2-bromoacetyl)piperidine-1-carboxylate (48.4 mg, 157.9 μmol) in t-BuOH (2 mL) was added 5-(2,8-dimethylimidazo[1,2-b]pyridazin-6-yl)-3-methylpyridin-2-amine (40 mg, 157.9 μmol) and NaHCO₃ (39.8 mg, 473.7 μmol) and the mixture was stirred at 80° C. for 16 h. The mixture was concentrated in vacuo to give the residue. The residue was dissolved in 4 M HCl in dioxane (0.39 mL) and DCM (2 mL). The mixture was stirred at 25° C. for 2 h. The mixture was concentrated in vacuo and purified by preparative HPLC (Column: Sunfire C18 100×19 mm, 5 mm; Mobile phase A: MeCN; Mobile phase B: H2O; Modifier: 0.1% TFA) to give 2,8-dimethyl-6-(8-methyl-2-(piperidin-4-yl)imidazo[1,2-a]pyridin-6-yl)imidazo[1,2-b]pyridazine trifluoroacetic acid slat (29.7 mg, 39.6% yield) as a white solid. MS: m/z 361.3 [M+H]⁺. RT: 0.72 min (Method 3)

### Example 37—Compound 556

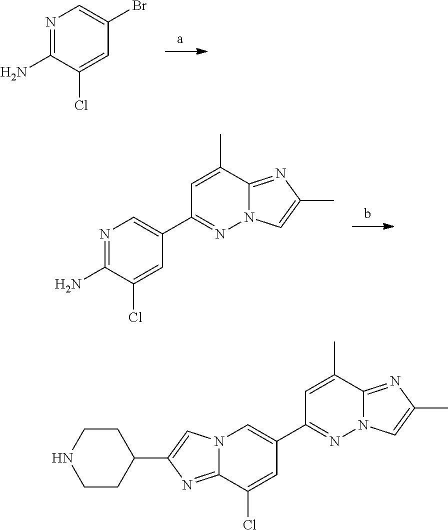 [SMILES: Nc1ncc(Br)cc1Cl ; Cc1cn2nc(-c3cnc(N)c(Cl)c3)cc(C)c2n1 ; Cc1cn2nc(-c3cc(Cl)c4nc(C5CCNCC5)cn4c3)cc(C)c2n1]

Step a: To a solution of 5-bromo-3-chloropyridin-2-amine (250 mg, 1.21 mmol) and (2,8-dimethylimidazo[1,2-b]pyridazin-6-yl)boronic acid (329.1 mg, 1.21 mmol) in dioxane (4.6 mL) and water (1.6 mL) was added Cs₂CO₃ (1.18 g, 3.62 mmol) and Pd(dppf)Cl₂ (98.4 mg, 120.5 μmol). The mixture was stirred under nitrogen at 90° C. for 16 hours. The mixture was extracted with EtOAc (15 mL×3) and dried with Na₂SO₄. The combined organic phase was filtered and concentrated under reduced pressure. The residue was purified by flash silica gel chromatography (0% to 20% MeOH:DCM) to give 3-chloro-5-(2,8-dimethylimidazo[1,2-b]pyridazin-6-yl)pyridin-2-amine (283.6 mg, 1.04 mmol, 86% yield) as a yellow solid. MS: m/z 274.1 [M+H]⁺.

Step b: To a solution of tert-butyl 4-(2-bromoacetyl)piperidine-1-carboxylate (44.8 mg, 146.1 μmol) in t-BuOH (2 mL) was added 3-chloro-5-(2,8-dimethylimidazo[1,2-b]pyridazin-6-yl)pyridin-2-amine (40 mg, 146.1 μmol) and NaHCO₃ (36.8 mg, 438.4 μmol) and the mixture was stirred at 80° C. for 16 h. The mixture was concentrated in vacuo to give the residue. The residue was dissolved in 4 M HCl in dioxane (0.37 mL) and DCM (2 mL). The mixture was stirred at 25° C. for 2 h. The mixture was concentrated in vacuo and purified by preparative HPLC (Column: Sunfire C18 100×19 mm, 5 mm; Mobile phase A: MeCN; Mobile phase B: H₂O; Modifier: 0.1% TFA) to give 6-(8-chloro-2-(piperidin-4-yl)imidazo[1,2-a]pyridin-6-yl)-2,8-dimethylimidazo[1,2-b]pyridazine trifluoroacetic acid slat (2.2 mg, 3.0% yield) as a white solid. MS: m/z 381.2 [M+H]⁺. RT: 0.81 min (Method 3)

### Example 38—Compound 535

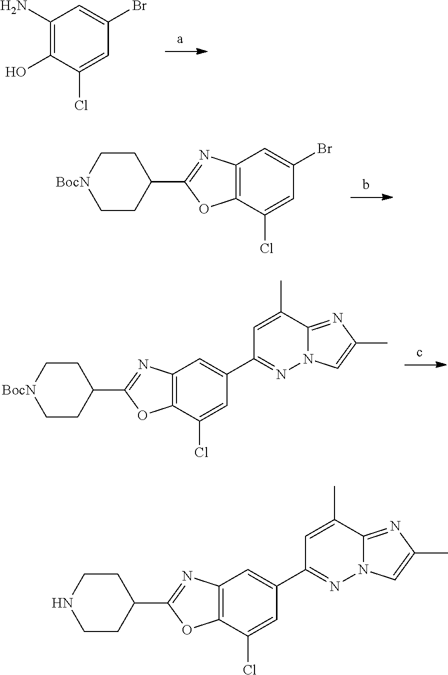

Step a: A mixture of 2-amino-4-bromo-6-chloro-phenol (500 mg, 2.25 mmol) and 1-tert-butoxycarbonylpiperidine-4-carboxylic acid (515.3 mg, 2.3 mmol) in PPA (5 mL) was stirred at 160° C. for 14 h. The mixture was poured into cold water, the mixture is neutralized with sodium bicarbonate and the pH was adjusted to around 8 by progressively adding TEA (22.7 mg, 224.8 umol, 31.33 μL), then the mixture was added tert-butoxycarbonyl tert-butyl carbonate (490.5 mg, 2.3 mmol, 516.3 μL), and then extracted by ethyl acetate (3×10 mL). The combined organic layers were dried over Na₂SO₄, filtered and concentrated. The crude material was purified on silica gel column chromatography (from PE/EtOAc=10/1 to 1/1) to yield tert-butyl 4-(5-bromo-7-chloro-1,3-benzoxazol-2-yl)piperidine-1-carboxylate (400 mg, 962.2 μmol, 42.81% yield) as brown solid. MS: m/z 415.1 [M+H]⁺.

Step b: To a solution of tert-butyl 4-(5-bromo-7-chloro-1,3-benzoxazol-2-yl)piperidine-1-carboxylate (90 mg, 216.5 μmol) and 2,8-dimethyl-6-(4,4,5,5-tetramethyl-1,3,2-dioxaborolan-2-yl)imidazo[1,2-b]pyridazine (60 mg, 219.7 μmol) in dioxane (4.5 mL) and water (0.5 mL) was added Pd(dppf)Cl₂ (31.7 mg, 43.3 μmol) and K₂CO₃ (59.8 mg, 433.0 μmol), the mixture was degassed with N₂ for 3 times and it was stirred at 80° C. for 2 h. Then the mixture was dissolved in MeOH and filtered, which was purified with prep-HPLC (Column: Welch Xtimate C18 150×25 mm×5 pm; Mobile Phase: from 60% to 90% of water (NH₄HCO₃)—CAN) to give tert-butyl 4-[7-chloro-5-(2,8-dimethylimidazo[1,2-b]pyridazin-6-yl)-1,3-benzoxazol-2-yl]piperidine-1-carboxylate (30 mg, 62.2 μmol, 28.8% yield) as white solid. MS: m/z 482.3 [M+H]⁺.

Step c: To a solution of tert-butyl 4-[7-chloro-5-(2,8-dimethylimidazo[1,2-b]pyridazin-6-yl)-1,3-benzoxazol-2-yl]piperidine-1-carboxylate (30 mg, 62.2 μmol) in Hexafluoroisopropanol (5 mL) was added TFA (35.5 mg, 311.2 μmol, 23.8 μL), the mixture was stirred at 25° C. for 1 h. Then the mixture was dissolved in MeCN and filtered, which was purified with prep-HPLC (Column: Welch Xtimate C18 150×25 mm×5 pm; Mobile Phase: from 25% to 55% of water (NH₄HCO₃)-CAN) to give 7-chloro-5-(2,8-dimethylimidazo[1,2-b]pyridazin-6-yl)-2-(4-piperidyl)-1,3-benzoxazole (8.5 mg, 22.3 μmol, 35.8% yield) as white solid. MS: m/z 382.1 [M+H]⁺. RT: 1.57 min (Method 10)

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 534 | Starting material: 8-fluoro-2-methyl-6-(4,4,5,5-tetramethyl-1,3,2- |
| | dioxaborolan-2-yl)imidazo[1,2-a]pyridine |
| | MS: m/z 385.1 [M + H]⁺ ; RT: 0.973 min (Method 10) |

### Example 39—Compound 533

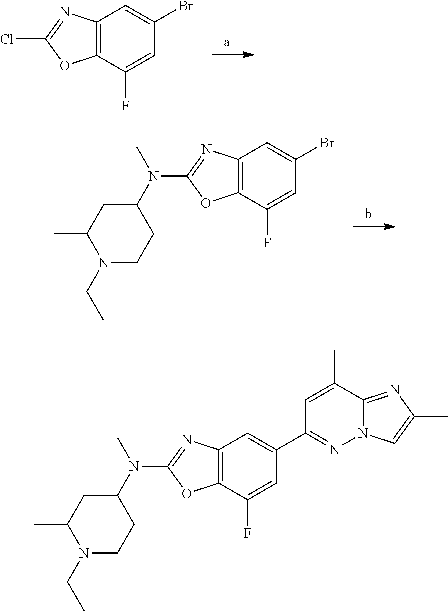

Step a: rel-(2R,4R)-1-Ethyl-N,2-dimethyl-piperidin-4-amine (41.2 mg, 263.5 μmol) and NEt₃ (48.5 mg, 479.1 μmol, 66.8 μL) were added to a mixture of 5-bromo-2-chloro-7-fluoro-1,3-benzoxazole (60 mg, 239.6 μmol) in DCM (1.5 mL) at 0° C., the reaction was stirred 2 h at r.t., quenched with water, extracted with DCM (2×10 mL), dried with MgSO₄, filtered and concentrated, rel-5-bromo-N-((2S,4S)-1-ethyl-2-methylpiperidin-4-yl)-7-fluoro-N-methylbenzo[d]oxazol-2-amine was obtained and used as crude in next step.

Step b: A mixture of rel-5-bromo-N-((2S,4S)-1-ethyl-2-methylpiperidin-4-yl)-7-fluoro-N-methylbenzo[d]oxazol-2-amine (237.7 μmol), 2,8-dimethyl-6-(4,4,5,5-tetramethyl-1,3,2-dioxaborolan-2-yl)imidazo[1,2-b]pyridazine (64.9 mg, 237.7 μmol), Pd(dppf)Cl₂ DCM (19.4 mg, 23.8 μmol), dicesium; carbonate (232.3 mg, 713.0 μmol) in dioxane (1.9 mL) and water (475.3 μL) was stirred under N₂ at 90° C. for 4 h. The residue was filtered through celite/MgSO₄ (DCM/EtOAc eluent), concentrated. The mixture was purified with column chromatography (Column: Sunfire C18 100×19 mm, 5 mm; Mobile phase A: MeCN; Mobile phase B: H2O; Modifier: 0.1% TFA) to obtain rel-5-(2,8-dimethylimidazo[1,2-b]pyridazin-6-yl)-N-((2S,4S)-1-ethyl-2-methylpiperidin-4-yl)-7-fluoro-N-methylbenzo[d]oxazol-2-amine (24.9 mg, 45.2 μmol, 19% yield, trifluoroacetic acid). MS: m/z 437.3 [M+H]⁺. RT: 1.15 min (Method 3)

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 532 | Starting material: N-methyl-2-morpholinoethan-1-amine and 8-fluoro-2- |
| | methyl-6-(4,4,5,5-tetramethyl-1,3,2-dioxaborolan-2-yl)imidazo[1,2- |
| | a]pyridine |
| | MS: m/z 428.1 [M + H]⁺ ]⁺; RT: 1.09 min (Method 3) |

### Example 40—Compound 468

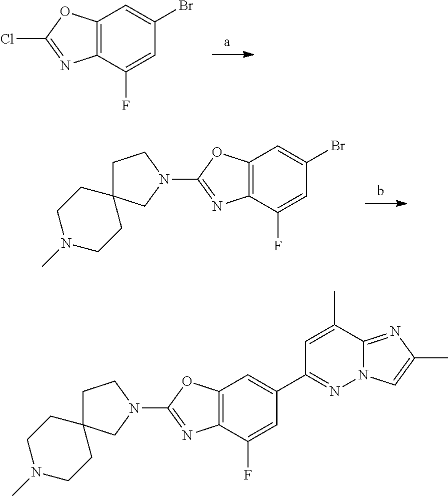

Step a: To a solution of 6-bromo-2-chloro-4-fluoro-1,3-benzoxazole (200 mg, 798.6 μmol) and 8-methyl-2,8-diazaspiro[4.5]decane (123.2 mg, 798.6 μmol) in dioxane (8 mL) was added TEA (242.4 mg, 2.40 mmol, 333.9 μL). The mixture was stirred at 100° C. for 2 hours. The reaction was filtered and concentrated. The residue was purified by flash silica gel chromatography (MeOH/EtOAc=0/100 to 20/80) to yield 6-bromo-4-fluoro-2-(8-methyl-2,8-diazaspiro[4.5]decan-2-yl)benzo[d]oxazole (250 mg, 678.9 μmol, 85% yield) as a yellow solid. MS: m/z 369.8 [M+H]⁺.

Step b: A mixture of 6-bromo-4-fluoro-2-(8-methyl-2,8-diazaspiro[4.5]decan-2-yl)benzo[d]oxazole (55.2 mg, 150 μmol), 2,8-dimethyl-6-(4,4,5,5-tetramethyl-1,3,2-dioxaborolan-2-yl)imidazo[1,2-b]pyridazine (40.9 mg, 150 μmol), Pd(dppf)Cl₂·CH₂Cl₂ (12.2 mg, 15.0 μmol), dicesium carbonate (450 mg, 146 μmol) in dioxane (1.9 mL) and water (475.3 μL) was stirred under N₂ at 90° C. for 4 h. The residue was filtered through celite/MgSO₄ (DCM/EtOAc eluent), concentrated. The mixture was purified by column chromatography (Column: Sunfire CS 100×19 mm, 5 mm; Mobile phase A: MeCN; Mobile phase B: H2O; Modifier: 0.1% TFA) to obtain 6-(2,8-dimethylimidazo[1,2-b]pyridazin-6-yl)-4-fluoro-2-(8-methyl-2,8-diazaspiro[4.5]decan-2-yl)benzo[d]oxazole, trifluoroacetic acid (18.0 mg, 32.8 Mmol, 27.6% yield, trifluoroacetic acid). MS: m/z 437.3 [M+H]⁺. ¹H NMR (600 MHz, DMSO-d₆) δ ppm 1.72-1.78 (m, 1H) 1.83-1.94 (m, 4H) 2.08 (t, J=7.25 Hz, 1H) 2.43-2.47 (m, 3H) 2.60-2.64 (m, 3H) 2.78-2.85 (m, 3H) 3.06-3.14 (m, 2H) 3.40 (br d, J=11.83 Hz, 2H) 3.50 (s, 1H) 3.68 (s, 1H) 3.72 (br t, J=7.06 Hz, 1H) 3.76 (br t, J=7.06 Hz, 1H) 7.77-7.83 (m, 1H) 7.88 (br d, J=1.14 Hz, 1H) 7.96-8.04 (m, 1H) 8.15 (s, 1H) 9.47 (br s, 1H).

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 486 | Starting material: Rac-(2R,4R)-1-ethyl-N,2-dimethyl-piperidin-4-amine |
| | MS: m/z 437.3 [M + H]⁺ ]⁺; RT: 0.49 min (Method 4) |
| 478 | Starting material: 1-cyclobutylpiperidin-4-amine |
| | MS: m/z 435.3 [M + H]⁺ ]⁺; RT: 0.44 min (Method 4) |
| 494 | Starting material: 4-(azetidin-1-yl)piperidine |
| | MS: m/z 421.2 [M + H]⁺ ]⁺; RT: 0.92 min (Method 10) |
| 491 | Starting material: N,1-dimethylpyrrolidin-3-amine |
| | MS: m/z 409.1 [M + H]⁺ ]⁺; RT: 0.92 min (Method 3) |
| 492 | Starting material: 4-ethyl-N,N-dimethyl-piperidin-4-amine |
| | MS: m/z 437.2 [M + H]⁺ ]⁺; RT: 0.97 min (Method 3) |
| 490 | Starting material: 1-(3-piperidyl)piperidine |
| | MS: m/z 449.2 [M + H]⁺ ]⁺; RT: 1.03 min (Method 3) |
| 493 | Starting material: 7-methyl-1,7-diazaspiro[3.5]nonane |
| | MS: m/z 421.1 [M + H]⁺ ]⁺; RT: 0.96 min (Method 3) |
| 500 | Starting material: N,N-dimethylpiperidin-3-amine |
| | MS: m/z 409.1 [M + H]⁺ ]⁺; RT: 0.93 min (Method 3) |
| 475 | Starting material: N,N-dimethylpiperidin-4-amine |
| | MS: m/z 409.3 [M + H]⁺ ]⁺; RT: 0.97 min (Method 3) |
| 479 | Starting material: 1-ethylpiperidin-4-amine |
| | MS: m/z 409.3 [M + H]⁺ ]⁺; RT: 0.99 min (Method 3) |
| 487 | Starting material: 1-ethyl-N,2-dimethyl-piperidin-4-amine |
| | MS: m/z 437.3 [M + H]⁺ ]⁺; RT: 0.49 min (Method 4) |
| 480 | Starting material: 1-(2-methoxyethyl)piperidin-4-amine |
| | MS: m/z 439.3 [M + H]⁺ ]⁺; RT: 1.00 min (Method 3) |
| 489 | Starting material: 1-cyclopropyl-N-methyl-N-[[(3S)-pyrrolidin-3- |
| | yl]methyl]methanamine |
| | MS: m/z 450.2 [M + H]⁺ ]⁺; RT: 0.90 min (Method 3) |
| 485 | Starting material: tert-butyl-4-amino-2,2-dimethyl-piperidine-1-carboxylate |
| | MS: m/z 409.3 [M + H]⁺ ]⁺; RT: 0.98 min (Method 3) |
| 484 | Starting material: tert-butyl-4-amino-2,6-dimethyl-piperidine-1-carboxylate |
| | MS: m/z 439.3 [M + H]⁺ ]⁺; RT: 1.03 min (Method 3) |
| 482 | Starting material: 1,2,3,5,6,7,8,8a-octahydroindolizin-7-amine |
| | MS: m/z 421.3 [M + H]⁺ ]⁺; RT: 1.00 min (Method 3) |
| 483 | Starting material: 1-[[pyrrolidin-3-yl]methyl]pyrrolidine |
| | MS: m/z 435.3 [M + H]⁺ ]⁺; RT: 1.00 min (Method 3) |
| 481 | Starting material: 1-[tetrahydrofuran-3-yl]piperidin-4-amine |
| | MS: m/z 451.3 [M + H]⁺ ]⁺; RT: 0.99 min (Method 3) |
| 488 | Starting material: 4-ethyl-N,N-dimethyl-piperidin-4-amine |
| | MS: m/z 437.3 [M + H]⁺ ]⁺; RT: 0.49 min (Method 4) |
| 477 | Starting material: 4-pyrrolidin-1-ylpiperidine |
| | MS: m/z 435.3 [M + H]⁺ ]⁺; RT: 1.02 min (Method 3) |
| 502 | Starting material: 4-ethyl-N,N-dimethyl-piperidin-4-amine |
| | MS: m/z 437.3 [M + H]⁺ ]⁺; RT: 0.49 min (Method 4) |
| 501 | Starting material: 2-methyl-2,7-diazaspiro[4.4]nonane |
| | MS: m/z 421.2 [M + H]⁺ ]⁺; RT: 0.94 min (Method 3) |
| 476 | Starting material: 1-[pyrrolidin-3-yl]pyrrolidine |
| | MS: m/z 421.3 [M + H]⁺ ]⁺; RT: 0.98 min (Method 3) |
| 469 | Starting material: 1-isopropyl-N-methyl-piperidin-4-amine |
| | MS: m/z 437.2 [M + H]⁺ ]⁺; RT: 1.0. min (Method 3) |
| 470 | Starting material: 2-methyl-2,8-diazaspiro[4.5]decane |
| | MS: m/z 435.2 [M + H]⁺ ]⁺; RT: 1.03 min (Method 3) |
| 471 | Starting material: 1-ethyl-2-methyl-piperidin-4-amine |
| | MS: m/z 423.2 [M + H]⁺ ]⁺; RT: 0.98 min (Method 3) |
| 503 | Starting material: 1-ethyl-N,2-dimethyl-piperidin-4-amine |
| | MS: m/z 437.2 [M + H]⁺ ]⁺; RT: 1.03 min (Method 3) |
| 473 | Starting material: N,1,2-trimethylpiperidin-4-amine |
| | MS: m/z 423.2 [M + H]⁺ ]⁺; RT: 0.99 min (Method 3) |
| 474 | Starting material: N,N-dimethyl-1-(3-methylpyrrolidin-3-yl)methanamine |
| | MS: m/z 423.2 [M + H]⁺ ]⁺; RT: 0.97 min (Method 3) |
| 472 | Starting material: 2-methyl-2,7-diazaspiro[3.4]octane |
| | MS: m/z 407.2 [M + H]⁺ ]⁺; RT: 0.91 min (Method 3) |
| 504 | Starting material: N,2,2,6,6-pentamethylpiperidin-4-amine, 2-methyl-6- |
| | (4,4,5,5-tetramethyl-1,3,2-dioxaborolan-2-yl)imidazo[1,2-b]pyridazine |
| | MS: m/z 437.1 [M + H]⁺ ]⁺; RT: 1.03 min (Method 3) |
| 466 | Starting material: 5-methyl-2,3,3a,4,6,6a-hexahydro-1H-pyrrolo[3,4- |
| | c]pyrrole |
| | MS: m/z 407.2 [M + H]⁺ ]⁺; RT: 0.95 min (Method 3) |
| 467 | Starting material: N,2,2,6,6-pentamethylpiperidin-4-amine |
| | MS: m/z 451.3 [M + H]⁺ ]⁺; RT: 1.15 min (Method 3) |
| 499 | Starting material: (4,6-dimethylpyrazolo[1,5-a]pyrazin-2-yl)boronic acid |
| | MS: m/z 435.2 [M + H]⁺; RT: 0.933 min (Method 10) |
| | ¹H NMR (400 MHz, METHANOL-d₄) δ ppm = 1.78-1.91 (m, 4H), 2.03 (t, J = |
| | 7.0 Hz, 2H), 2.45 (s, 3H), 2.61 (s, 3H), 2.71 (s, 3H), 2.76-2.90 (m, 2H), |
| | 3.00-2.95 (m, 2H), 3.57 (s, 2H), 3.70-3.75 (m, 2H), 7.20 (s, 1H), 7.59 (d, |
| | J = 11.6 Hz, 1H), 7.72 (s, 1H), 8.21 (s, 1H), 8.54 (s, 1H). |
| 521 | Starting material: tert-butyl 4-aminopiperidine-1-carboxylate |
| | MS: m/z 395.1 [M + H]⁺ ]⁺; RT: 0.685 min (Method 10) |
| 522 | Starting material: tert-butyl 4-aminopiperidine-1-carboxylate |
| | MS: m/z 435.2 [M + H]⁺ ]⁺; RT: 0.778 min (Method 10) |
| 519 | Starting material: N-methyl-3-pyrrolidin-1-yl-cyclobutanamine |
| | MS: m/z 435.1 [M + H]⁺ ]⁺; RT: 1.730 min (Method 10) |
| 516 | Starting material: tert-butyl 2,9-diazaspiro[5.5]undecane-2-carboxylate |
| | MS: m/z 449.2 [M + H]⁺ ]⁺; RT: 1.03 min (Method 10) |
| 518 | Starting material: tert-butyl (1S,5S)-3,6-diazabicyclo[3.2.0]heptane-3- |
| | carboxylate |
| | MS: m/z 393.2 [M + H]⁺ ]⁺; RT: 1.707 min (Method 10) |
| 523 | Starting material: tert-butyl 4-aminopiperidine-1-carboxylate |
| | MS: m/z 423.2 [M + H]⁺ ]⁺; RT: 0.745 min (Method 10) |
| 526 | Starting material: rac-(3R)-1-cyclobutylpyrrolidin-3-amine |
| | MS: m/z 421.2 [M + H]⁺ ]⁺; RT: 1.042 min (Method 8) |

### Example 41—Compound 507

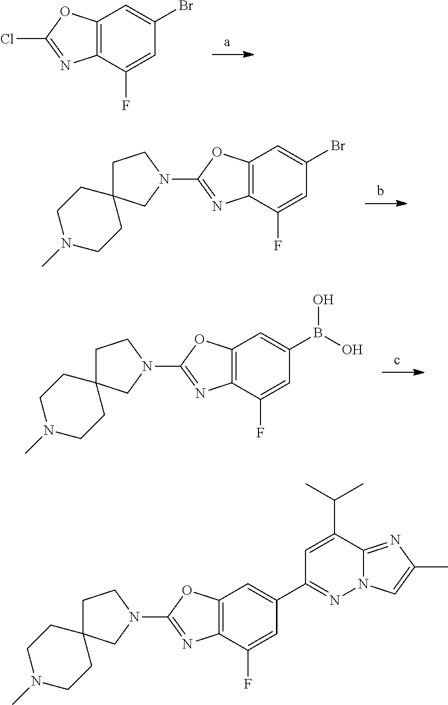

Step a: To a solution of 6-bromo-2-chloro-4-fluoro-1,3-benzoxazole (200 mg, 798.6 mol) and 8-methyl-2,8-diazaspiro[4.5]decane (123.2 mg, 798.6 μmol) in dioxane (8 mL) was added TEA (242.4 mg, 2.40 mmol, 333.9 μL). The mixture was stirred at 100° C. for 2 hours. The reaction was filtered and concentrated. The residue was purified by flash silica gel chromatography (MeOH/EtOAc=0/100 to 20/80) to yield 6-bromo-4-fluoro-2-(8-methyl-2,8-diazaspiro[4.5]decan-2-yl)benzo[d]oxazole (250 mg, 678.9 μmol, 85% yield) as a yellow solid. MS: m/z 369.8 [M+H]⁺.

Step b: To a solution of 6-bromo-4-fluoro-2-(8-methyl-2,8-diazaspiro[4.5]decan-2-yl)-1,3-benzoxazole (200 mg, 543.1 μmol) and 4,4,5,5-tetramethyl-2-(4,4,5,5-tetramethyl-1,3,2-dioxaborolan-2-yl)-1,3,2-dioxaborolane (179.3 mg, 706.1 μmol) in Dioxane (20 mL). Then KOAc (159.9 mg, 1.6 mmol) and Pd(dppf)Cl₂ (39.7 mg, 54.3 μmol) were added to the solution. The mixture was stirred under nitrogen at 90° C. for 2 hours. The crude compound was used into the next step without further purification.

Step c: To a solution of 4-fluoro-2-(8-methyl-2,8-diazaspiro[4.5]decan-2-yl)-6-(4,4,5,5-tetramethyl-1,3,2-dioxaborolan-2-yl)-1,3-benzoxazole (60 mg, 144.5 μmol) and 6-chloro-8-(difluoromethyl)-2-methyl-imidazo[1,2-b]pyridazine (31.44 mg, 144.5 μmol) in H2O (0.8 mL) and dioxane (4 mL) was added Pd(dppf)Cl₂ (10.6 mg, 14.5 μmol) and K₂CO₃ (59.9 mg, 433.4 μmol). The mixture was stirred under nitrogen at 90° C. for 2 hours. The mixture was extracted with EtOAc (25 mL×3) and dried with Na₂SO₄. The combined organic phase was filtered and concentrated under reduced pressure. The residue was purified by prep-HPLC purification (Column: Welch Xtimate C18 150×25 mm×5 pm; Mobile Phase: from 25% to 55% of water (NH₄HCO₃)-CAN) to yield 6-(8-(difluoromethyl)-2-methylimidazo[1,2-b]pyridazin-6-yl)-4-fluoro-2-(8-methyl-2,8-diazaspiro[4.5]decan-2-yl)benzo[d]oxazole (10.6 mg, 22.5 μmol, 15.6% yield) was obtained as a white solid. MS: m/z 471.2 [M+H]⁺. ¹H NMR (400 MHz, METHANOL-d₄) δ ppm=1.80-1.90 (s, 4H), 2.05 (t, J=6.7 Hz, 2H), 2.50 (s, 3H), 2.65 (s, 3H), 2.86-3.05 (m, 4H), 3.60 (s, 2H), 3.79 (t, J=6.9 Hz, 2H), 7.12-7.39 (m, 1H), 7.75 (d, J=11.3 Hz, 1H), 7.88 (d, J=10.6 Hz, 2H), 8.03 (s, 1H), 8.54 (s, 1H).

Using the procedure described for Example 12 above, additional compounds described herein were prepared by substituting the appropriate starting material in step a, suitable reagents and reaction conditions, obtaining compounds such as those selected from:

| Cpd ID | Data |
|---|---|
| 496 | Starting material: N,N-dimethylpiperidin-4-amine and 6-chloro-2,8- |
| | dimethyl-[1,2,4]triazolo[1,5-b]pyridazine |
| | MS: m/z 410.2 [M + H]⁺; RT: 1.073 min (Method 7) |
| 497 | Starting material: N,N-dimethylpiperidin-4-amine and 2-bromo-4,6- |
| | dimethylpyrazolo[1,5-a]pyrazine |
| | MS: m/z 409.2 [M + H]⁺; RT: 1.049 min (Method 10) |
| | ¹H NMR: (400 MHz, METHANOL-d₄) δ ppm = 1.62-1.72 (m, 2H), 2.00- |
| | 2.10 (m, 2H), 2.43 (s, 6H), 2.52 (s, 3H), 2.65-2.72 (m, 1H), 2.76 (s, 3H), |
| | 3.20-3.30 (m, 2H), 4.40-4.50 (m, 2H), 6.89 (s, 1H), 7.55 (dd, J = 1.2, 11.0 |
| | Hz, 1H), 7.70 (d, J = 11.2 Hz, 1H), 8.07 (s, 1H), 8.44 (br s, 1H). |
| 498 | Starting material: 6-chloro-2,8-dimethyl-[1,2,4]triazolo[1,5-b]pyridazine |
| | MS: m/z 436.2 [M + H]⁺; RT: 1.572 min (Method 8) |
| 506 | Starting material: N,N-dimethylpiperidin-4-amine and 6-chloro-2-methyl-8- |
| | (trifluoromethyl)imidazo[1,2-b]pyridazine |
| | MS: m/z 463.2 [M + H]⁺; RT: 1.186 min (Method 10) |
| | ¹H NMR (400 MHz, METHANOL-d₄) δ ppm = 1.62-1.71 (m, 2H), 2.05- |
| | 2.15 (m, 2H), 2.50 (s, 6H), 2.51 (s, 3H), 2.78-2.86 (m, 1H), 3.20-3.30 (m, |
| | 2H), 4.40-4.50 (m, 2H), 7.76 (d, J = 10.4 Hz, 1H), 7.90 (d, J = 1.6 Hz, 1H), |
| | 7.98 (s, 1H), 8.07 (s, 1H). |
| 505 | Starting material: 6-chloro-8-methoxy-2-methyl-[1,2,4]triazolo[1,5- |
| | b]pyridazine |
| | MS: m/z 452.2 [M + H]⁺; RT: 1.757 min (Method 10) |
| 495 | Starting material: N,N-dimethylpiperidin-4-amine |
| | MS: m/z 445.1 [M + H]⁺; RT: 1.552 min (Method 8) |
| 517 | Starting material: 8-methyl-2,8-diazaspiro[4.5]decane |
| | MS: m/z 472.2 [M + H]⁺ ]⁺; RT: 1.337 min (Method 10) |
| 520 | Starting material: 8-methyl-2,8-diazaspiro[4.5]decane |
| | MS: m/z 489.2 [M + H]⁺ ]⁺; RT: 1.627 min (Method 10) |
| 573 | Starting material: 1-cyclobutylpiperidin-4-amine and 8-methoxy-2-methyl- |
| | 6-(4,4,5,5-tetramethyl-1,3,2-dioxaborolan-2-yl)imidazo[1,2-b]pyridazine |
| | MS: m/z 451.2 [M + H]⁺ ]⁺; RT: 2.17 min (Method 10) |

### Example 42—Compound 545

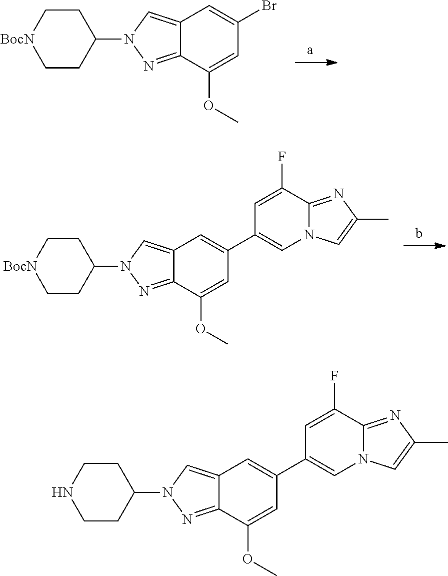 [SMILES: COc1cc(Br)cc2cn(C3CCN(C(=O)OC(C)(C)C)CC3)nc12 ; COc1cc(-c2cc(F)c3nc(C)cn3c2)cc2cn(C3CCN(C(=O)OC(C)(C)C)CC3)nc12 ; COc1cc(-c2cc(F)c3nc(C)cn3c2)cc2cn(C3CCNCC3)nc12]

Step a: tert-butyl 4-(5-bromo-7-methoxy-2H-indazol-2-yl)piperidine-1-carboxylate (75 mg, 182 mmol) was mixed with 8-fluoro-2-methyl-6-(4,4,5,5-tetramethyl-1,3,2-dioxaborolan-2-yl)imidazo[1,2-a]pyridine (55 mg, 182 mmole) and Cs₂CO₃ (119 mg, 365 mmole). The solids were dissolved with Dioxane (2.4 ml) and water (0.6 ml), degassed with N₂ and SPhos Pd G2 (13.1 mg, 18 mmole) was added. The reaction was heated in the microwave at 90 C for 1 h. the resulting was purified by flash chromatography with a gradient of 0-100% EtOAc-Heptane to afford the title compound. MS: m/z 480.2 [M+H]⁺]⁺; RT: 0.67 min (Method 4)

Step b: To a solution of tert-butyl 4-(5-(8-fluoro-2-methylimidazo[1,2-a]pyridin-6-yl)-7-methoxy-2H-indazol-2-yl)piperidine-1-carboxylate (161 mg, 337 mmole) in DCM, TFA (25 uL) was added. The reaction was heated to 45° C. for 2 hours. The reaction was quenched with sodium bicarb and product was extracted with chloroform:IPA (3:1). The organic layer was concentrated in vacuo to afford the title compound. MS: m/z 380.1 [M+H]⁺; RT: 0.83 min (Method 3)

Using the procedure described for Example 17 above, additional compounds described herein were prepared by substituting the appropriate boronic ester starting material in step b, suitable reagents and reaction conditions, obtaining compounds such as those selected from:

| Cpd ID | Data |
|---|---|
| 543 | Starting material: 7-fluoro-2-methyl-5-(4,4,5,5-tetramethyl-1,3,2- |
| | dioxaborolan-2-yl)-2H-indazole |
| | MS: m/z 368.1 [M + H]⁺; RT: 0.87 min (Method 3) |
| 544 | Starting material: 2,7-dimethyl-5-(4,4,5,5-tetramethyl-1,3,2-dioxaborolan- |
| | 2-yl)-2H-indazole |
| | MS: m/z 376.2 [M + H]⁺; RT: 1.18 min (Method 3) |
| 546 | Starting material: 2-methyl-6-(4,4,5,5-tetramethyl-1,3,2-dioxaborolan-2- |
| | yl)imidazo[1,2-a]pyridine-8-carbonitrile |
| | MS: m/z 387.3 [M + H]⁺; RT: 1.52 min (Method 1) |
| 547 | Starting material: 2,8-dimethyl-6-(4,4,5,5-tetramethyl-1,3,2-dioxaborolan- |
| | 2-yl)-[1,2,4]triazolo[1,5-a]pyridine |
| | MS: m/z 377.3 [M + H]⁺; RT: 1.45 min (Method 1) |

### Example 43—Compound 548

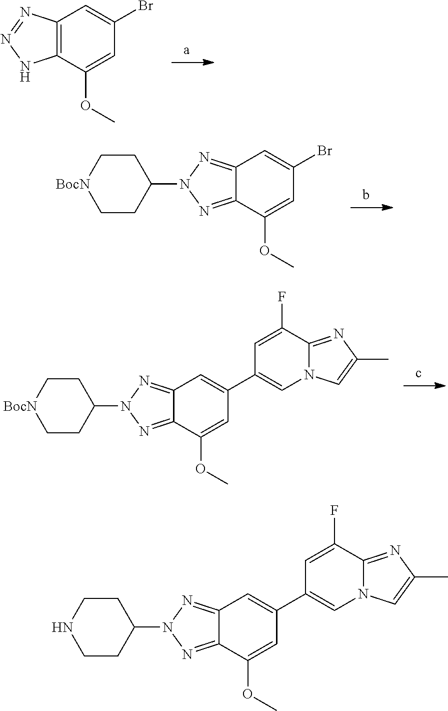

Step a: 6-bromo-4-methoxy-2H-benzotriazole (114.02 mg, 0.5 mmol, 1.0 eq.) was dissolved in Tetrahydrofuran (3.5 mL, 0.14 M) before tert-butyl 4-hydroxypiperidine-1-carboxylate (100 mg, 500 umol, 1.0 eq.), DIAD (101.11 mg, 500 umol, 1.0 eq.), and Triphenylphosphine (131315 mg, 500 umol, 1.0 eq.) were added. The solution then stirred at RT for 16 hours before it was concentrated then dry loaded onto normal phase silica for purification. Reaction purified via 0-100% EtOAc:heptane over 7.0 minutes. Product elute around 70% EtOAc. Identified fractions were combined and concentrated to obtain tert-butyl 4-(6-bromo-4-methoxy-benzotriazol-2-yl)piperidine-1-carboxylate (92.3 mg, 23.79% yield). MS: m/z 357.0 [M+H−tBu]⁺; RT: 1.03 min (Method 4)

Step b: tert-butyl 4-(6-bromo-4-methoxy-benzotriazol-2-yl)piperidine-1-carboxylate (46.15 mg, 112.21 umol 1.0 eq.), 8-fluoro-2-methyl-6-(4,4,5,5-tetramethyl-1,3,2-dioxaborolan-2-yl)imidazo[1,2-a]pyridine (30.98 mg, 112.21 umol, 1.0 eq.), Cesium carbonate (73.12 mg, 224.31 umol, 2.0 eq.), and Pd(dppf)Cl2 CH2Cl2 (9.16 mg, 11.22 umol, 0.1 eq.) were dissolved in water (1 mL, 0.56 M) and Dioxane (1 mL, 0.56 M) before being heated to 100° C. for 16 hours. The solution was then telescoped forward crude. Obtained tert-butyl 4-[6-(8-fluoro-2-methyl-imidazo[1,2-a]pyridin-6-yl)-4-methoxy-benzotriazol-2-yl]piperidine-1-carboxylate (assumed 100% yield) in solution. MS: m/z 481.3 [M+H]⁺; RT: 0.72 min (Method 4)

Step c: tert-butyl 4-[6-(8-fluoro-2-methyl-imidazo[1,2-a]pyridin-6-yl)-4-methoxy-benzotriazol-2-yl]piperidine-1-carboxylate (53.82 mg, 112 umol, 1.0 eq.) was dissolved in DCM (1 mL, 0.11 M) before 4M HCl in dioxanes (440.84 mg, 1.12 mmol, 10.0 eq.) was added. The solution then stirred at 60° C. for 16 hours before being concentrated and taken back up in a minimal amount of DMSO, filtered, and purified via HPLC purification (column: XSelect CSH Prep C18 5 um OBD 19×100 mm; Mobile phase A: MeCN; Mobile phase B: H2O, Modifier: 0.1% NH4OH). Obtained 6-(8-fluoro-2-methyl-imidazo[1,2-a]pyridin-6-yl)-4-methoxy-2-(4-piperidyl)benzotriazole (5.2 mg, 12% yield) as a brown solid. MS: m/z 481.1 [M+H]⁺; RT: 0.83 min (Method 3)

### Example 44—Compound 550

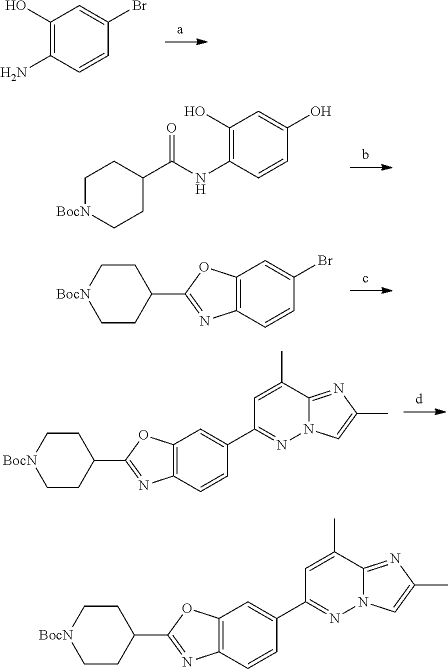

Step a: To a solution of 2-amino-5-bromo-phenol (500 mg, 2.66 mmol) and 1-tert-butoxycarbonylpiperidine-4-carboxylic acid (609.7 mg, 2.66 mmol) in DCM (10 mL) was added HATU (1.01 g, 2.66 mmol) and TEA (403.6 mg, 3.99 mmol, 556 μL). The mixture was stirred under nitrogen at 25° C. for 16 hours. The mixture was concentrated under reduced pressure. The residue was extracted with EtOAc (20 mL×3), washed with brine, dried over Na₂SO₄, filtered, and concentrated in vacuo. The residue was purified by silica gel chromatography (20-30% EtOAc/petroleum ether) to afford the desired product tert-butyl 4-[(4-bromo-2-hydroxy-phenyl)carbamoyl]piperidine-1-carboxylate (500 mg, 1.25 mmol, 47.1% yield) as a yellow oil.

Step b: To a solution of tert-butyl 4-[(4-bromo-2-hydroxy-phenyl)carbamoyl]piperidine-1-carboxylate (2 g, 5.01 mmol) in THF (30 mL) was added triphenylphosphane (1.45 g, 5.51 mmol) and isopropyl (NE)-N-isopropoxycarbonyliminocarbamate (1.11 g, 5.51 mmol, 1.1 mL). The mixture was stirred under nitrogen at 25° C. for 8 hours. The mixture was concentrated under reduced pressure. The residue was extracted with EtOAc (50 mL×3), washed with brine, dried over Na₂SO₄, filtered, and concentrated in vacuo. The residue was purified by silica gel chromatography (20-30% EtOAc/petroleum ether) to afford the desired product tert-butyl 4-(6-bromo-1,3-benzoxazol-2-yl)piperidine-1-carboxylate (1.6 g, 4.20 mmol, 83.8% yield) as a purple oil.

Step c: To a solution of tert-butyl 4-(6-bromo-1,3-benzoxazol-2-yl)piperidine-1-carboxylate (70 mg, 183.60 μmol) and 2,8-dimethyl-6-(4,4,5,5-tetramethyl-1,3,2-dioxaborolan-2-yl)imidazo[1,2-b]pyridazine (50.2 mg, 183.60 μmol) in Dioxane (2 mL) was added Pd(dppf)Cl2 (26.9 mg, 36.72 μmol) and K₂CO₃ (50.8 mg, 367.20 μmol). The mixture was stirred under nitrogen at 90° C. for 16 hours. The mixture was purified by prep-HPLC (column: Boston Green ODS 150×30 mm×5 um; condition: water (0.05% HCl)-ACN; begin B: 35; end B: 55; Gradient Time (min): 10; 100% B Hold Time (min): 2; FlowRate (ml/min): 25) to give tert-butyl 4-[6-(2,8-dimethylimidazo[1,2-b]pyridazin-6-yl)-1,3-benzoxazol-2-yl]piperidine-1-carboxylate (35 mg, 78.21 μmol, 42.6% yield) as a yellow solid. MS: m/z 448.2 [M+H]⁺.

Step d: A solution of tert-butyl 4-[6-(2,8-dimethylimidazo[1,2-b]pyridazin-6-yl)-1,3-benzoxazol-2-yl]piperidine-1-carboxylate (30 mg, 67.03 μmol) in HCl/Dioxane (2 mL) was stirred under nitrogen at 25° C. for 16 hours. The mixture was purified by prep-HPLC (column: Welch Xtimate C18 150×25 mm×5 um; condition: water (10 mM NH4HCO₃)-ACN; begin B: 12; end B: 32; Gradient Time (min): 10; 100% B Hold Time (min): 2; FlowRate (ml/min): 25) to give 6-(2,8-dimethylimidazo[1,2-b]pyridazin-6-yl)-2-(4-piperidyl)-1,3-benzoxazole (6 mg, 17.27 μmol, 25.8% yield) as a white solid. MS: m/z 348.2 [M+H]⁺. RT: 1.3 min (Method 10).

### Example 45—Compound 293

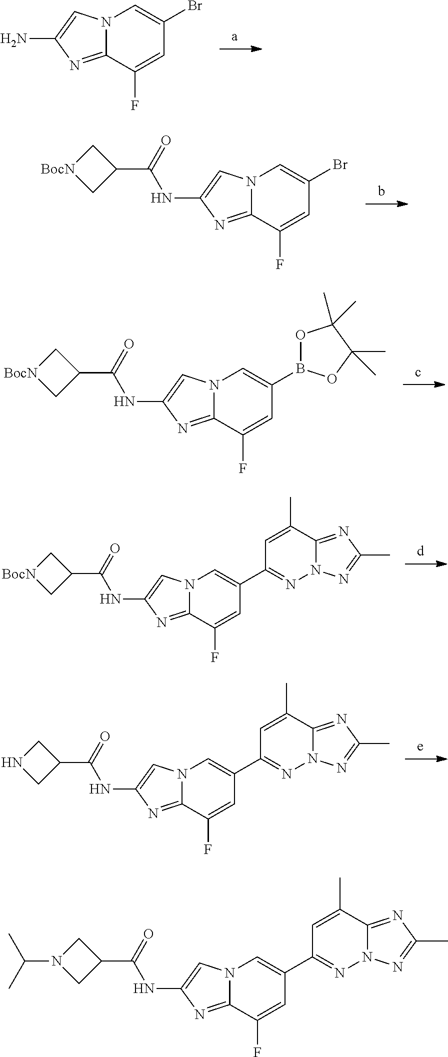

Step a: To a solution of 6-bromo-8-fluoro-imidazo[1,2-a]pyridin-2-amine (30 mg, 130.41 μmol) and 1-tert-butoxycarbonylazetidine-3-carboxylic acid (28.87 mg, 143.46 umol) in pyridine (2 mL) was added EDCI (37.50 mg, 195.62 μmol). The reaction was stirred at 90° C. for 2 h. Then the mixture was filtered and concentrated. The residue was triturated with MeOH (5 mL) and H₂O (15 mL) at 20° C. for 15 min. The mixture was filtered to give tert-butyl 3-[(6-bromo-8-fluoro-imidazo[1,2-a]pyridin-2-yl)carbamoyl]azetidine-1-carboxylate (30 mg, 68.21 μmol, 52.30% yield) as a brown solid. MS: m/z 413.1 [M+H]⁺.

Step b: To a solution of tert-butyl 3-[(6-bromo-8-fluoro-imidazo[1,2-a]pyridin-2-yl)carbamoyl]azetidine-1-carboxylate (76 mg, 183.91 μmol) in Dioxane (5 mL) was added KOAc (54.15 mg, 551.74 μmol), 4,4,5,5-tetramethyl-2-(4,4,5,5-tetramethyl-1,3,2-dioxaborolan-2-yl)-1,3,2-dioxaborolane (46.70 mg, 183.91 μmol) and Pd(dppf)Cl₂ (20.19 mg, 27.59 μmol). The mixture was stirred at 90° C. for 16 hr under N₂. The mixture was quenched with water (30.0 mL) and extracted with EA (20.0 mL×3). The combined organic layers were washed with brine (20.0 mL), dried over Na₂SO₄, filtered and concentrated under vacuum (low temperature) to give the crude. The crude was purified by chromatography column on silica gel (PE/EA=1/1=1/0 to 10/1) to give tert-butyl 3-[[8-fluoro-6-(4,4,5,5-tetramethyl-1,3,2-dioxaborolan-2-yl)imidazo[1,2-a]pyridin-2-yl]carbamoyl]azetidine-1-carboxylate (60 mg, 130.35 μmol, 70.9% yield) as yellow solid.

Step c: To a solution of tert-butyl 3-[[8-fluoro-6-(4,4,5,5-tetramethyl-1,3,2-dioxaborolan-2-yl)imidazo[1,2-a]pyridin-2-yl]carbamoyl]azetidine-1-carboxylate (100 mg, 217.25 μmol) in dioxane (5 mL) and water (0.5 mL) was added 6-chloro-2,8-dimethyl-[1,2,4]triazolo[1,5-b]pyridazine (47.61 mg, 260.70 μmol), Pd(dppf)Cl2(15.90 mg, 21.72 μmol) and K₂CO₃ (90.07 mg, 651.74 μmol) under N₂. The reaction was stirred at 90° C. for 2 h. The mixture was then concentrated before water (80 mL) was added. The mixture was extracted with EtOAc (50 mL×3). The combined organic layers were washed with brine (50 mL), dried over Na₂SO₄, filtered and concentrated in vacuo to give the crude product. The crude product was purified by Combi-Flash (DCM/MeOH=10/1) to yield tert-butyl 3-[[6-(2,8-dimethyl-[1,2,4]triazolo[1,5-b]pyridazin-6-yl)-8-fluoro-imidazo[1,2-a]pyridin-2-yl]carbamoyl]azetidine-1-carboxylate (60 mg, 124.87 μmol, 57.5% yield) as brown solid. MS: m/z 481.3 [M+H]⁺.

Step d: To a solution of tert-butyl 3-[[6-(2,8-dimethyl-[1,2,4]triazolo[1,5-b]pyridazin-6-yl)-8-fluoro-imidazo[1,2-a]pyridin-2-yl]carbamoyl]azetidine-1-carboxylate (10 mg, 20.81 μmol) in DCM (1 mL) was added TFA (0.5 mL). The reaction was stirred at 20° C. for 1 h. The mixture was concentrated in vacuo to give N-[6-(2,8-dimethyl-[1,2,4]triazolo[1,5-b]pyridazin-6-yl)-8-fluoro-imidazo[1,2-a]pyridin-2-yl]azetidine-3-carboxamide (10 mg, crude) as yellow solid. MS: m/z 381.1 [M+H]⁺. RT: 1.453 min (Method 10)

Using the procedure described for Example 21 above, additional compounds described herein were prepared by substituting the appropriate boronic ester in step a, suitable reagents, and reaction conditions, obtaining compounds such as those selected from:

| Cpd ID | Data |
|---|---|
| 292 | Starting material: 5-chloro-2,7-dimethyl-pyrazolo[4,3-b]pyridine |
| | MS: m/z 422.1 [M + H]⁺; RT: 1.88 min (Method 8) |
| 295 | Starting material: 2-bromo-4,6-dimethyl-pyrazolo[1,5-a]pyrazine |
| | MS: m/z 422.2 [M + H]⁺; RT: 1.73 min (Method 10) |
| 297 | Starting material: 2,8-dimethyl-6-(4,4,5,5-tetramethyl-1,3,2-dioxaborolan-2- |
| | yl)-[1,2,4]triazolo[1,5-b]pyridazine |
| | MS: m/z 409.1 [M + H]⁺ ]⁺; RT: 1.75 min (Method 10) |
| 298 | Starting material: 2,8-dimethyl-6-(4,4,5,5-tetramethyl-1,3,2-dioxaborolan-2- |
| | yl)-[1,2,4]triazolo[1,5-b]pyridazine |
| | MS: m/z 395.1 [M + H]⁺ ]⁺; RT:1.710. min (Method 10) |
| 299 | Starting material: 1-methylpiperidine-4-carboxylic acid |
| | MS: m/z 423.1 [M + H]⁺ ]⁺; RT: 1.674 min (Method 8) |
| 300 | Starting material: (R)-2-(1-(tert-butoxycarbonyl)piperidin-2-yl)acetic acid |
| | and 6-bromo-2,8-dimethyl-[1,2,4]triazolo[1,5-b]pyridazine |
| | MS: m/z 436.1 [M + H]⁺ ]⁺; RT: 0.969 min (Method 8) |
| 301 | Starting material: (S)-2-(1-(tert-butoxycarbonyl)piperidin-2-yl)acetic acid |
| | and 6-bromo-2,8-dimethyl-[1,2,4]triazolo[1,5-b]pyridazine |
| | MS: m/z 436.1 [M + H]⁺ ]⁺; RT: 1.442 min (Method 8) |
| 302 | Starting material: (R)-2-(1-(tert-butoxycarbonyl)piperidin-2-yl)acetic acid |
| | MS: m/z 437.2 [M + H]⁺ ]⁺; RT: 1.237 min (Method 10) |
| 303 | Starting material: (S)-2-(1-(tert-butoxycarbonyl)piperidin-2-yl)acetic acid |
| | MS: m/z 437.2 [M + H]⁺ ]⁺; RT: 1.247 min (Method 10) |
| 304 | Starting material: 1-methylpiperidine-4-carboxylic acid and 6-chloro-8- |
| | methoxy-2-methyl-[1,2,4]triazolo[1,5-b]pyridazine |
| | MS: m/z 1.398 [M + H]⁺ ]⁺; RT: 439.2 min (Method 10) |
| 305 | Starting material: 6-chloro-8-(difluoromethyl)-2-methyl-[1,2,4]triazolo[1,5- |
| | b]pyridazine, paraformaldehyde, trans-3-(tert- |
| | butoxycarbonylamino)cyclobutanecarboxylic acid |
| | MS: m/z 459.1 [M + H]⁺ ]⁺; RT: 1.48 min (Method 10) |
| 306 | Starting material: 6-chloro-8-(difluoromethyl)-2-methyl-[1,2,4]triazolo[1,5- |
| | b]pyridazine, paraformaldehyde, cis-3-(tert- |
| | butoxycarbonylamino)cyclobutanecarboxylic acid |
| | MS: m/z 459.1 [M + H]⁺ ]⁺; RT: 1.50 min (Method 10) |
| 307 | Starting material: (2,8-dimethyl-[1,2,4]triazolo[1,5-b]pyridazin-6-yl)boronic |
| | acid, paraformaldehyde, trans-3-(tert- |
| | butoxycarbonylamino)cyclobutanecarboxylic acid |
| | MS: m/z 423.2 [M + H]⁺ ]⁺; RT: 1.02 min (Method 10) |
| 308 | Starting material: (2,8-dimethyl-[1,2,4]triazolo[1,5-b]pyridazin-6-yl)boronic |
| | acid, paraformaldehyde, cis-3-(tert- |
| | butoxycarbonylamino)cyclobutanecarboxylic acid |
| | MS: m/z 423.2 [M + H]⁺ ]⁺; RT: 1.49 min (Method 10) |

### Example 46—Compound 324

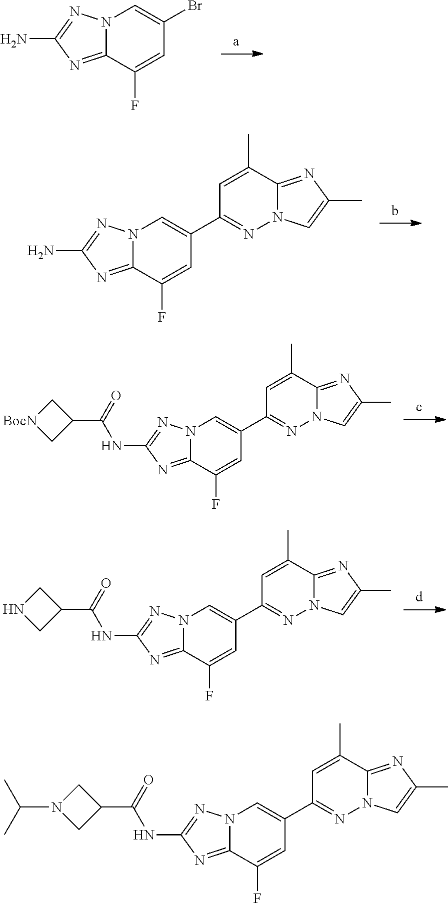

Step a: To a solution of 6-bromo-8-fluoro-[1,2,4]triazolo[1,5-a]pyridin-2-amine (300 mg, 1.30 mmol) in dioxane (10 mL) and water (1 mL) was added 2,8-dimethyl-6-(4,4,5,5-tetramethyl-1,3,2-dioxaborolan-2-yl)imidazo[1,2-b]pyridazine (354.69 mg, 1.30 mmol), Pd(dppf)Cl₂ (95.02 mg, 129.86 μmol) and K₂CO₃ (538.42 mg, 3.90 mmol) under N₂. The reaction was stirred at 90° C. for 2 h. The mixture was concentrated and then water (80 mL) was added. The mixture was extracted with EtOAc (50 mL×3). The combined organic layers were washed with brine (50 mL), dried over Na₂SO₄, filtered, and concentrated in vacuo. The residue was washed with water (30 mL), evacuated under vacuum to yield 6-(2,8-dimethylimidazo[1,2-b]pyridazin-6-yl)-8-fluoro-[1,2,4]triazolo[1,5-a]pyridin-2-amine (300 mg, 1.01 mmol, 77.71% yield) as brown solid.

Step b: A mixture of 6-(2,8-dimethylimidazo[1,2-b]pyridazin-6-yl)-8-fluoro-[1,2,4]triazolo[1,5-a]pyridin-2-amine (50 mg, 168.19 μmol) and 1-tert-butoxycarbonylazetidine-3-carboxylic acid (33.84 mg, 168.19 μmol) in pyridine (3 mL) was added T₃P (3 mL) stirred at 25° C. for 2 h. The reaction mixture was quenched by water (50 mL). The aqueous layer was separated and extracted with EtOAc (20 mL×3). The combined organic layers were dried over Na₂SO₄, filtered and concentrated to afford the crude product. The crude material was purified on silica gel column chromatography (from PE/EtOAc=5/1 to 2/1) to yield tert-butyl 3-[[6-(2,8-dimethylimidazo[1,2-b]pyridazin-6-yl)-8-fluoro-[1,2,4]triazolo[1,5-a]pyridin-2-yl]carbamoyl]azetidine-1-carboxylate (28.9 mg, 60.15 μmol, 35.76% yield) as a yellow solid. MS: m/z 481.2 [M+H]⁺.

Step c: tert-Butyl 3-[[6-(2,8-dimethylimidazo[1,2-b]pyridazin-6-yl)-8-fluoro-[1,2,4]triazolo[1,5-a]pyridin-2-yl]carbamoyl]azetidine-1-carboxylate (20 mg, 41.62 μmol) was added to HCl/EA (5 mL) in one portion at 25° C. The mixture was stirred for 16 hours. The reaction mixture was concentrated to afford the crude product N-[6-(2,8-dimethylimidazo[1,2-b]pyridazin-6-yl)-8-fluoro-[1,2,4]triazolo[1,5-a]pyridin-2-yl]azetidine-3-carboxamide (15 mg, 39.43 μmol, 94.74% yield) as a yellow solid. The crude was used for next step directly.

Step d: A mixture of N-[6-(2,8-dimethylimidazo[1,2-b]pyridazin-6-yl)-8-fluoro-[1,2,4]triazolo[1,5-a]pyridin-2-yl]azetidine-3-carboxamide (10 mg, 26.29 μmol), acetone (4.58 mg, 78.87 μmol, 5.79 μL) in EtOH (5 mL). After 30 mins, sodium triacetoxyborohydride (16.72 mg, 78.87 μmol) was added in one portion at 25° C. The reaction was stirred at 25° C. for 3 hours. The mixture was concentrated and purified by pre-HPLC (Column Welch Xtimate C18 150×25 mm×5 pm; Condition water (FA)-ACN; Begin B 0; End B 30; Gradient Time (min) 11; 100% B Hold Time (min) 2; FlowRate (ml/min) 25) to give N-[6-(2,8-dimethylimidazo[1,2-b]pyridazin-6-yl)-8-fluoro-[1,2,4]triazolo[1,5-a]pyridin-2-yl]-1-isopropyl-azetidine-3-carboxamide (2.3 mg, 5.44 μmol, 20.71% yield) as a white solid. MS: m/z 423.2 [M+H]⁺; RT: 1.583 (Method 10).

Using the procedure described for Example 21 above, additional compounds described herein were prepared by substituting the appropriate boronic ester in step a, suitable reagents, and reaction conditions, obtaining compounds such as those selected from:

| Cpd ID | Data |
|---|---|
| 315 | Starting material: 1-tert-butoxycarbonylpiperidine-3-carboxylic acid |
| | MS: m/z 409.3 [M + H]⁺ ]⁺; RT: 0.82 min (Method 3) |
| 316 | Starting material: 1-tert-butoxycarbonylpiperidine-3-carboxylic acid |
| | MS: m/z 409.3 [M + H]⁺ ]⁺; RT: 0.83 min (Method 3) |
| 317 | Starting material: 1-methylpiperidine-4-carboxylic acid, 2,8-dimethyl-6- |
| | (4,4,5,5-tetramethyl-1,3,2-dioxaborolan-2-yl)-[1,2,4]triazolo[1,5- |
| | b]pyridazine |
| | MS: m/z 424.2 [M + H]⁺ ]⁺; RT: 1.35 min (Method 10) |
| 318 | Starting material: 5-tert-butoxycarbonyl-5-azaspiro[2.3]hexane-2-carboxylic |
| | acid |
| | MS: m/z 407.1 [M + H]⁺ ]⁺; RT: 1.59 min (Method 8) |
| 319 | Starting material: (3S)-1-tert-butoxycarbonyl-4,4-dimethyl-pyrrolidine-3- |
| | carboxylic acid |
| | MS: m/z 423.1 [M + H]⁺ ]⁺; RT: 1.65 min (Method 8) |
| 320 | Starting material: 2-tert-butoxycarbonyl-2-azabicyclo[2.1.1]hexane-4- |
| | carboxylic acid |
| | MS: m/z 407.1 [M + H]⁺ ]⁺; RT: 1.56 min (Method 8) |
| 321 | Starting material: 1-tert-butoxycarbonylazepane-3-carboxylic acid |
| | MS: m/z 423.1 [M + H]⁺ ]⁺; RT: 1.64 min (Method 8) |
| 322 | Starting material: 1-tert-butoxycarbonyl-2-methyl-piperidine-4-carboxylic |
| | acid |
| | MS: m/z 423.1 [M + H]⁺ ]⁺; RT: 1.62 min (Method 8) |
| 323 | Starting material: 4-tert-butoxycarbonyl-4-azaspiro[2.4]heptane-7-carboxylic |
| | acid |
| | MS: m/z 421.1 [M + H]⁺ ]⁺; RT: 1.61 min (Method 8) |
| 325 | Starting material: 1-tert-butoxycarbonylpiperidine-3-carboxylic acid, |
| | paraformaldehyde |
| | MS: m/z 423.2 [M + H]⁺ ]⁺; RT: 1.08 min (Method 10) |
| 326 | Starting material: 1-tert-butoxycarbonylpiperidine-3-carboxylic acid, |
| | paraformaldehyde |
| | MS: m/z 423.0 [M + H]⁺ ]⁺; RT: 0.63 min (Method 7) |
| 327 | Starting material: 1-methylpiperidine-4-carboxylic acid |
| | MS: m/z 423.1 [M + H]⁺ ]⁺; RT: 1.58 min (Method 8) |
| 328 | Starting material: 2-[rac-(2S)-1-tert-butoxycarbonyl-2-piperidyl]acetic acid, |
| | paraformaldehyde |
| | MS: m/z 437.2 [M + H]⁺ ]⁺; RT: 1.60 min (Method 10) |
| 329 | Starting material: 2-[rac-(2S)-1-tert-butoxycarbonylpyrrolidin-2-yl]acetic |
| | acid, paraformaldehyde |
| | MS: m/z 423.1 [M + H]⁺ ]⁺; RT: 1.57 min (Method 10) |
| 330 | Starting material: rac-(7R)-4-tert-butoxycarbonyl-4-azaspiro[2.5]octane- |
| | 7-carboxylic acid |
| | MS: m/z 435.2 [M + H]⁺ ]⁺; RT: 0.75 min (Method 7) |

### Example 47—Compound 361 and 367

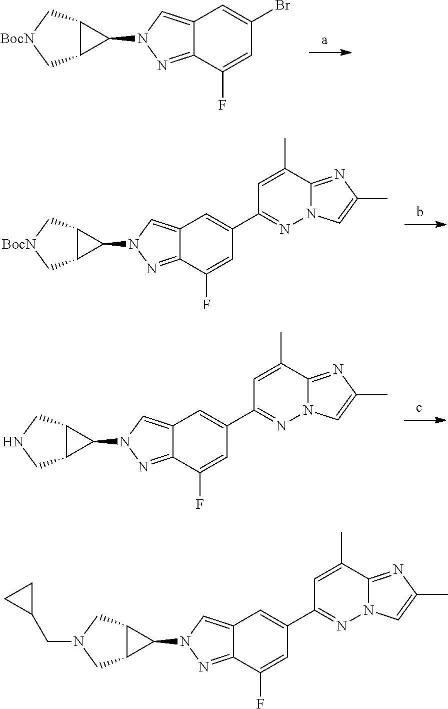 [SMILES: CC(C)(C)OC(=O)N1C[C@@H]2[C@H](C1)[C@H]2n1cc2cc(Br)cc(F)c2n1 ; Cc1cn2nc(-c3cc(F)c4nn([C@H]5[C@@H]6CN(C(=O)OC(C)(C)C)C[C@@H]65)cc4c3)cc(C)c2n1 ; Cc1cn2nc(-c3cc(F)c4nn([C@H]5[C@@H]6CNC[C@@H]65)cc4c3)cc(C)c2n1 ; Cc1cn2nc(-c3cc(F)c4nn([C@H]5[C@@H]6CN(CC7CC7)C[C@@H]65)cc4c3)cc(C)c2n1]

Step a: tert-butyl 6-(5-bromo-7-fluoro-indazol-2-yl)-3-azabicyclo[3.1.0]hexane-3-carboxylate (100 mg, 252.36 umol, 1.0 eq.) was dissolved in Dioxane (1.51 mL, 0.14M) with 2,8-dimethyl-6-(4,4,5,5-tetramethyl-1,3,2-dioxaborolan-2-yl)imidazo[1,2-b]pyridazine (75.82 mg, 277.60 umol, 1.1 eq.), and PdCl2(dppf)(15.03 mg, 25.24 umol, 0.1 eq.), Cs2CO3 (246.67 mg, 757.09 umol, 3.0 eq.). water (0.25 mL, 0.14M) was added and degassed with N2. The vial was sealed and microwaved to 90 C for 1 h. The organics were then injected to silica and purified by FCC using a gradient of 0-100% EtOAc-Heptane. Obtained tert-butyl 6-[5-(2,8-dimethylimidazo[1,2-b]pyridazin-6-yl)-7-fluoro-indazol-2-yl]-3-azabicyclo[3.1.0]hexane-3-carboxylate. MS: m/z 463.5 [M+H]⁺; RT 0.74 min (Method 4).

Step b: tert-butyl-6-[5-(2,8-dimethylimidazo[1,2-b]pyridazin-6-yl)-7-fluoro-indazol-2-yl]-3-azabicyclo[3.1.0]hexane-3-carboxylate (85.00 mg, 183.78 umol, 1.0 eq.) was dissolved in DCM (2 mL, 0.09M). Hydrochloric acid (67.01 mg, 1.84 mmol, 10.0 eq.) was added, and stirred at room temp 1 h. the reaction was concentrated, and part moved forward crude. Other part was purified via HPLC purification (Column: Sunfire C18 100×19 mm, 5 mm; Mobile phase A: MeCN; Mobile phase B: H2O; Modifier: 0.1% TFA). MS: m/z 363.3 [M+H]⁺; RT 1.72 min (Method 1).

Step c: 6-[2-[(1R,5S)-3-azabicyclo[3.1.0]hexan-6-yl]-7-fluoro-indazol-5-yl]-2,8-dimethyl-imidazo[1,2-b]pyridazine (35 mg, 96 umol) was dissolved in DCM (965 uL). Sodium cyanoborohydride (18 mg, 289.73 umol) and cyclopropanecarbaldehyde (7 mg, 96.58 umol, 7 uL) were mixed at room temperature over night. the resulting was diluted with water and extracted with EtOAc. Then purified by prep-HPLC to afford 6-[2-[(1R,5S)-3-(cyclopropylmethyl)-3-azabicyclo[3.1.0]hexan-6-yl]-7-fluoro-indazol-5-yl]-2,8-dimethyl-imidazo[1,2-b]pyridazine (11.6 mg, 27.85 μmol, 28.84% yield). MS: m/z 417.3 [M+H]⁺; RT 1.11 min (Method 4).

### Example 48—Compound 374 and/or 375

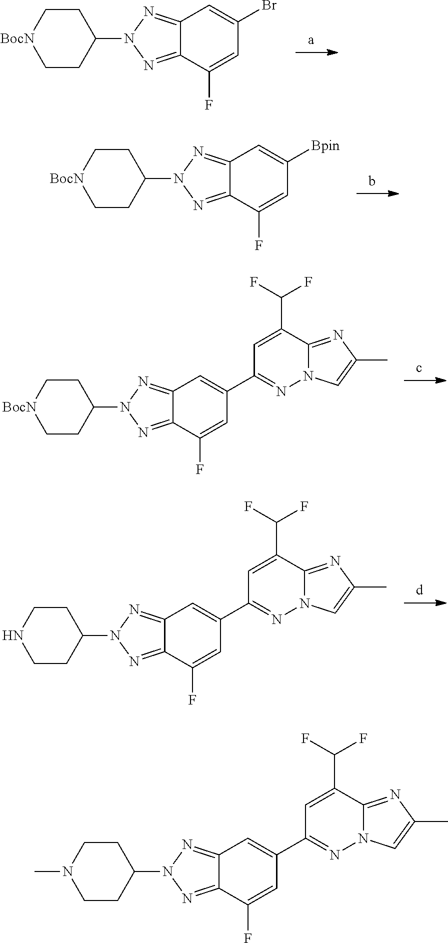

Step a: Tert-butyl 4-(6-bromo-4-fluoro-benzotriazol-2-yl)piperidine-1-carboxylate (362.06 mg, 734.53 umol) was dissolved in Dioxane (3.67 mL) before 4,4,5,5-tetramethyl-2-(4,4,5,5-tetramethyl-1,3,2-dioxaborolan-2-yl)-1,3,2-dioxaborolane (279.79 mg, 1.10 mmol) and Potassium acetate (144.17 mg, 1.47 mmol) were added. The solution was then sparged with nitrogen before Pd(dppf)Cl2 CH2Cl₂ (29.99 mg, 36.73 umol) was added. The solution then stirred at 100° C. for 1 hour before being concentrated in vacuo, injected crude onto normal phase, and purified via 0-25% MeOH:DCM. Product elutes at 15% MeOH over 12 minutes. Identified fractions were combined and concentrated to obtain tert-butyl 4-[4-fluoro-6-(4,4,5,5-tetramethyl-1,3,2-dioxaborolan-2-yl)benzotriazol-2-yl]piperidine-1-carboxylate (430.1 mg, 85.28% yield). MS: m/z 391.2 [M+H-tBu]⁺; RT 1.15 min (Method 4).

Step b: Tert-butyl 4-[4-fluoro-6-(4,4,5,5-tetramethyl-1,3,2-dioxaborolan-2-yl)benzotriazol-2-yl]piperidine-1-carboxylate (390.26 mg, 874.39 umol), 6-chloro-8-(difluoromethyl)-2-methyl-imidazo[1,2-b]pyridazine (313.22 mg, 1.05 mmol, Hydrobromide), and Cesium carbonate (1.14 g, 3.50 mmol) were dissolved in Dioxane (3.28 mL) and water (1.09 mL). The solution was then sparged with nitrogen before Pd(dppf)Cl2 CH2Cl₂ (71.41 mg, 87.44 umol) was added. The solution then stirred at 110° C. for 16 hours before it was concentrated, dry loaded onto normal phase silica, and purified via 0-25% MeOH:DCM over 12 minutes. Product elutes at 13% MeOH. Identified fractions were collected, combined, and concentrated to obtain tert-butyl 4-[6-[8-(difluoromethyl)-2-methyl-imidazo[1,2-b]pyridazin-6-yl]-4-fluoro-benzotriazol-2-yl]piperidine-1-carboxylate (214.9 mg, 48.03% yield). MS: m/z 501.2 [M+H]⁺; RT 0.69 min (Method 4).

Step c: Tert-butyl 4-[6-[8-(difluoromethyl)-2-methyl-imidazo[1,2-b]pyridazin-6-yl]-4-fluoro-benzotriazol-2-yl]piperidine-1-carboxylate (214.9 mg, 419.94 umol) was dissolved in DCM (908.87 uL) before HCl (4 M, 4.20 mmol, 1.05 mL) was added. The solution immediately smoked and became cloudy before being stirred at 40° C. for 16 hours after which a precipitate had formed. The solution was then diluted with diethyl ether and a further amount of precipitate formed that was filtered off. The precipitate was washed with diethyl ether a few times before being redissolved in DMSO, water, and methanol then filtered and purified via reversed phase basic conditions HPLC purification (column: XSelect CSH Prep C18 5 um OBD 19×100 mm; Mobile phase A: MeCN; Mobile phase B: H2O, Modifier: 0.1% NH4OH). Identified fractions were collected and concentrated then registered as is. Obtained 6-[8-(difluoromethyl)-2-methyl-imidazo[1,2-b]pyridazin-6-yl]-4-fluoro-2-(4-piperidyl)benzotriazole (99.1 mg, 57.62% yield) as a white powder. 1H NMR (400 MHz, METHANOL-d4) δ ppm 2.22-2.39 (m, 4H) 2.51-2.56 (m, 3H) 2.82-2.92 (m, 2H) 3.21-3.29 (m, 2H) 5.00-5.10 (m, 1H) 7.15-7.42 (m, 1H) 7.89-7.94 (m, 1H) 8.03-8.05 (m, 1H) 8.10-8.12 (m, 1H) 8.43-8.45 (m, 1H). MS: m/z 402.2 [M+H]⁺; RT 0.52 min (Method 4).

Step d: 6-[8-(difluoromethyl)-2-methyl-imidazo[1,2-b]pyridazin-6-yl]-4-fluoro-2-(4-piperidyl)benzotriazole (50 mg, 117.09 umol) was dissolved in Acetonitrile (537.93 uL) before TEA (23.70 mg, 234.19 umol) was added. The solution stirred for one minute before Paraformaldehyde (140.46 mg, 117.09 umol) and Acetic acid (35.16 mg, 585.47 umol) were added. The solution immediately smoked and stirred at RT for 10 minutes before Sodium cyanoborohydride (14.72 mg, 234.19 umol) was added. The solution then stirred for 30 minutes before it was concentrated before being taken back up in DMSO and filtered then injected crude onto reversed phase and purified via HPLC purification (Column: Sunfire C18 100×19 mm, 5 mm; Mobile phase A: MeCN; Mobile phase B: H2O; Modifier: 0.1% TFA) to obtain 6-[8-(difluoromethyl)-2-methyl-imidazo[1,2-b]pyridazin-6-yl]-4-fluoro-2-(1-methyl-4-piperidyl)benzotriazole (21.7 mg, 30.45% yield). 1H NMR (400 MHz, METHANOL-d4) δ ppm 2.56-2.57 (m, 3H) 2.59-2.65 (m, 2H) 2.70-2.77 (m, 2H) 2.99-3.01 (m, 3H) 3.37-3.43 (m, 2H) 3.58-3.64 (m, 1H) 3.76-3.83 (m, 2H) 7.31-7.34 (m, 1H) 7.98-8.02 (m, 1H) 8.14-8.17 (m, 1H) 8.19-8.21 (m, 1H) 8.50-8.53 (m, 1H). MS: m/z 416.2 [M+H]⁺; RT 0.55 min (Method 4).

Using the procedure described for Example 17 above, additional compounds described herein were prepared by substituting the appropriate boronic ester starting material in step b, suitable reagents and reaction conditions, obtaining compounds such as those selected from:

| Cpd ID | Data |
|---|---|
| 390 | Starting material: 6-chloro-2,8-dimethyl-[1,2,4]triazolo[1,5-b]pyridazine |
| | MS: m/z 367.2 [M + H]⁺ ]⁺; RT: 0.93 min (Method 10) |
| 391 | Starting material: 6-chloro-2-methyl-8-(trifluoromethyl)imidazo[1,2- |
| | b]pyridazine |
| | MS: m/z 420.1 [M + H]⁺ ]⁺; RT: 1.07 min (Method 10) |
| 392 | Starting material: 6-chloro-8-methoxy-2-methyl-[1,2,4]triazolo[1,5- |
| | b]pyridazine |
| | MS: m/z 383.1 [M + H]⁺ ]⁺; RT: 2.46 min (Method 8) |
| 389 | Starting material: 6-chloro-8-ethyl-2-methyl-imidazo[1,2-b]pyridazine |
| | MS: m/z 380.2 [M + H]⁺ ]⁺; RT: 1.36 min (Method 8) |
| 387 | Starting material: 6-chloro-8-cyclopropyl-2-methyl-imidazo[1,2- |
| | b]pyridazine |
| | MS: m/z 392.0 [M + H]⁺ ]⁺; RT: 0.89 min (Method 7) |
| 377 | Starting material: 6-chloro-8-(difluoromethyl)-2-methyl-imidazo[1,2- |
| | b]pyridazine, acetaldehyde |
| | MS: m/z 430.1 [M + H]⁺ ]⁺; RT: 2.30 min (Method 1) |
| 381 | Starting material: 5-chloro-2,7-dimethyl-pyrazolo[4,3-b]pyridine |
| | MS: m/z 366.2 [M + H]⁺; RT: 0.52 min (Method 4) |
| | 1H NMR (600 MHz, DMSO-d6) δ ppm 2.37-2.42 (m, 2 H) 2.47-2.49 (m, |
| | 2 H) 2.62-2.66 (m, 3 H) 3.19-3.25 (m, 2 H) 3.46-3.50 (m, 2 H) 4.22- |
| | 4.26 (m, 3 H) 5.27-5.33 (m, 1 H) 7.94-7.97 (m, 1 H) 8.11-8.17 (m, 1 H) |
| | 8.54-8.56 (m, 1 H) 8.66-8.69 (m, 1 H) |
| 388 | Starting material: 2-bromo-4,6-dimethyl-pyrazolo[1,5-a]pyrazine |
| | MS: m/z 366.3 [M + H]⁺; RT: 1.22 min (Method 3) |
| | 1H NMR (600 MHz, DMSO-d6) δ ppm 2.02-2.09 (m, 2 H) 2.18-2.23 (m, |
| | 2 H) 2.42-2.44 (m, 3 H) 2.68-2.70 (m, 1 H) 2.70-2.72 (m, 3 H) 2.72- |
| | 2.73 (m, 1 H) 3.08-3.12 (m, 2 H) 4.94-5.01 (m, 1 H) 7.64-7.66 (m, 1 H) |
| | 7.88-7.92 (m, 1 H) 8.40-8.43 (m, 1 H) 8.51-8.54 (m, 1 H) |
| 399 | Starting material: 6-chloro-8-(difluoromethyl)-2-methyl- |
| | [1,2,4]triazolo[1,5-b]pyridazine |
| | MS: m/z 403.2 [M + H]⁺ ]⁺; RT: 3.24 min (Method 8) |
| 408 | Starting material: tert-butyl-5-hydroxy-3,3a,4,5,6,6a-hexahydro-1H- |
| | cyclopenta[c]pyrrole-2-carboxylate |
| | MS: m/z 428.2 [M + H]⁺ ]⁺; RT: 1.30 min (Method 3) |
| 409 | Starting material: tert-butyl (6R)-6-hydroxy-2-azaspiro[3.4]octane-2- |
| | carboxylate |
| | MS: m/z 428.2 [M + H]⁺ ]⁺; RT: 1.32 min (Method 3) |
| 416 | Starting material: 5-bromo-2H-benzotriazole, 6-chloro-8-(difluoromethyl)- |
| | 2-methyl-imidazo[1,2-b]pyridazine |
| | MS: m/z 384.2 [M + H]⁺; RT: 2.13 min (Method 10) |
| 563 | Starting material: tert-butyl (7R)-7-hydroxy-5-oxa-2-azaspiro[3.4]octane- |
| | 2-carboxylate |
| | MS: m/z 430.2 [M + H]⁺; RT: 0.53 min (Method 4). |
| 569 | Starting material: 6-bromo-4-fluoro-2-(2-methylpiperidin-4-yl)-2H- |
| | benzo[d][1,2,3]triazole |
| | MS: m/z 416.2 [M + H]⁺; RT: 0.31 min (Method 9). |
| 570 | Starting material: 6-bromo-4-fluoro-2-(2-methylpiperidin-4-yl)-2H- |
| | benzo[d][1,2,3]triazole |
| | MS: m/z 416.2 [M + H]⁺; RT: 0.32 min (Method 9). |
| 571 | Starting material: 6-bromo-4-fluoro-2-(2-methylpiperidin-4-yl)-2H- |
| | benzo[d][1,2,3]triazole |
| | MS: m/z 416.2 [M + H]⁺; RT: 0.31 min (Method 9). |
| 572 | Starting material: 6-bromo-4-fluoro-2-(2-methylpiperidin-4-yl)-2H- |
| | benzo[d][1,2,3]triazole |
| | MS: m/z 416.0 [M + H]⁺; RT: 0.31 min (Method 9). |

### Example 48: Compound 380

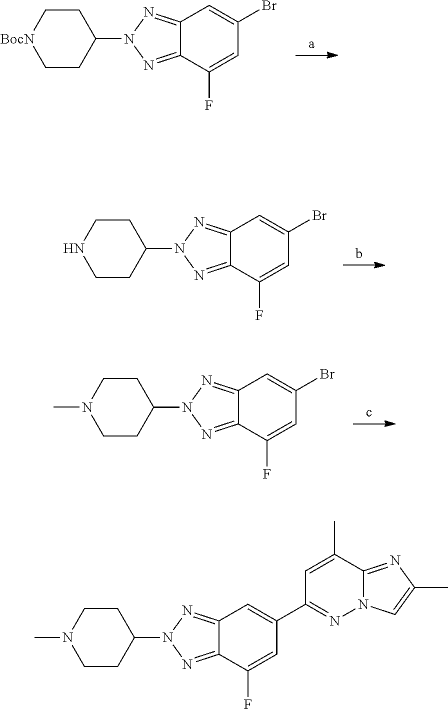

Step a: To a stirred solution of tert-butyl 4-(6-bromo-4-fluoro-benzotriazol-2-yl)piperidine-1-carboxylate (0.1 g, 250.47 umol, 1.0 eq.) in DCM (5 mL, 0.05M) was added HCl/EtOAc (35 mg, 1 mmol, 4 eq.). The reaction mixture was stirred at RT for 1 h. The mixture was filtered and concentrated to give a residue. The crude compound was used into the next step without further purification. Obtained 6-bromo-4-fluoro-2-(4-piperidyl)benzotriazole (0.05 g, 59.86% yield) was obtained as a white solid. MS: m/z 299.9 [M+H]⁺; RT 0.29 min (Method 9).

Step b: To a stirred solution of 6-bromo-4-fluoro-2-(4-piperidyl)benzotriazole (0.065 g, 217.29 umol, 1.0 eq.) in 1,2-Dichloroethane (1 mL, 0.2 M) and EtOH (5 mL, 0.04 M) was added Paraformaldehyde (260.65 mg, 217.29 umol, 1.0 eq.) and TEA (43.97 mg, 434.58 umol, 2.0 eq.). The mixture was stirred at RT for 10 min. Sodium triacetoxyborohydride (92.10 mg, 434.58 umol, 2.0 eq.) was then added. The mixture stirred at RT for 30 minutes before the mixture was filtered and concentrated to give a residue. Obtained 6-bromo-4-fluoro-2-(1-methyl-4-piperidyl)benzotriazole (35 mg, 110.98 umol, 51.07% yield, 99.3% purity) was obtained as a white solid. MS: m/z 314.8 [M+H]⁺; RT 0.32 min (Method 9).

Step c: To a stirred solution of 6-bromo-4-fluoro-2-(1-methyl-4-piperidyl)benzotriazole (20 mg, 63.86 umol, 1.0 eq.) in Dioxane (2.5 mL, 0.02 M) and water (0.5 mL, 0.02 M) was added cyclopentyl(diphenyl)phosphane; dichloropalladium; iron (46.73 mg, 63.86 umol, 0.1 eq.), K2CO3 (8.83 mg, 63.86 umol, 1.0 eq.) and 2,8-dimethyl-6-(4,4,5,5-tetramethyl-1,3,2-dioxaborolan-2-yl)imidazo[1,2-b]pyridazine (17.44 mg, 63.86 umol, 1.0 eq.). The reaction mixture was stirred at 95° C. for 12 h under N2. The mixture was filtered and concentrated to give a residue. The residue was purified by HPLC purification (column: XSelect CSH Prep C18 5 um OBD 19×100 mm; Mobile phase A: MeCN; Mobile phase B: H2O, Modifier: 0.1% NH4OH) to obtain 6-(2,8-dimethylimidazo[1,2-b]pyridazin-6-yl)-4-fluoro-2-(1-methyl-4-piperidyl)benzotriazole (2.8 mg, 7.34 umol, 11.50% yield, 99.5% purity) as a white solid. MS: m/z 380.0 [M+H]⁺; RT 0.26 min (Method 9).

### Example 49—Compound 339

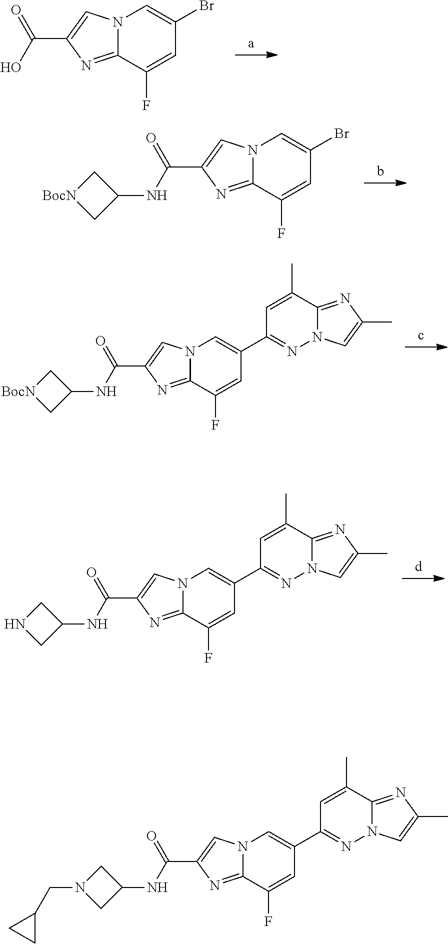

Step a: To a stirred solution of 6-bromo-8-fluoro-imidazo[1,2-a]pyridine-2-carboxylic acid (650 mg, 2.51 mmol) and tert-butyl 3-aminoazetidine-1-carboxylate (388.95 mg, 2.26 mmol) in DMF (10 mL) was added HATU (1.14 g, 3.01 mmol) and DIPEA (972.95 mg, 7.53 mmol, 1.31 mL). The reaction was stirred at 20° C. for 1 h. The residue was triturated with EtOAc (20 mL) at 20° C. for 30 min. tert-Butyl 3-[(6-bromo-8-fluoro-imidazo[1,2-a]pyridine-2-carbonyl)amino]azetidine-1-carboxylate (400 mg, 803.41 μmol, 32.02% yield) was obtained as a yellow solid.

Step b: To a stirred solution of tert-butyl 3-[(6-bromo-8-fluoro-imidazo[1,2-a]pyridine-2-carbonyl)amino]azetidine-1-carboxylate (150 mg, 362.98 μmol) and 2,8-dimethyl-6-(4,4,5,5-tetramethyl-1,3,2-dioxaborolan-2-yl)imidazo[1,2-b]pyridazine (99.14 mg, 362.98 μmol) in dioxane (10 mL) and H2O (2 mL) was added K₂CO₃ (150.50 mg, 1.09 mmol) and Pd(dppf)Cl₂ (26.56 mg, 36.30 μmol). The reaction mixture was stirred at 90° C. for 2 h. The mixture was filtered and concentrated. The residue was purified by flash silica gel chromatography (from DCM/MeOH=10/1 to 5/1, TLC: DCM/MeOH=10/1, Rf=0.66) to yield tert-butyl 3-[[6-(2,8-dimethylimidazo[1,2-b]pyridazin-6-yl)-8-fluoro-imidazo[1,2-a]pyridine-2-carbonyl]amino]azetidine-1-carboxylate (40 mg, 62.56 μmol, 17.24% yield, 75% purity) as a yellow solid. MS: m/z 480.2 [M+H]⁺; RT: 0.357 min (method 9).

Step c: To a solution of tert-butyl 3-[[6-(2,8-dimethylimidazo[1,2-b]pyridazin-6-yl)-8-fluoro-imidazo[1,2-a]pyridine-2-carbonyl]amino]azetidine-1-carboxylate (50 mg, 104.27 μmol) in EtOAc (2 mL) was added HCl/EtOAc (2 mL). The reaction was stirred at 20° C. for 2 h. The mixture was filtered and concentrated under reduced pressure to give N-(azetidin-3-yl)-6-(2,8-dimethylimidazo[1,2-b]pyridazin-6-yl)-8-fluoro-imidazo[1,2-a]pyridine-2-carboxamide (40 mg, crude) as a yellow solid. MS: m/z 380.1 [M+H]⁺; RT: 0.243 min (method 9).

Step d: To a solution of N-(azetidin-3-yl)-6-(2,8-dimethylimidazo[1,2-b]pyridazin-6-yl)-8-fluoro-imidazo[1,2-a]pyridine-2-carboxamide (20 mg, 52.72 μmol) in DCE/EtOH (5 mL) was added cyclopropanecarbaldehyde (5.54 mg, 79.07 μmol, 5.91 μL) and TEA (16.00 mg, 158.15 μmol, 22.04 μL). The reaction was stirred at 25° C. for 15 mins before sodium triacetoxyborohydride (4.97 mg, 79.07 μmol) was added. The reaction was stirred at 25° C. for 12 hours. The mixture was filtered and concentrated. The residue was purified by prep-HPLC (FA condition) to give N-[1-(cyclopropylmethyl)azetidin-3-yl]-6-(2,8-dimethylimidazo[1,2-b]pyridazin-6-yl)-8-fluoro-imidazo[1,2-a]pyridine-2-carboxamide (4.49 mg, 10.03 μmol, 19.04% yield) as a white solid. MS: m/z 434.1 [M+H]⁺; RT: 1.432 min (method 10).

Using the procedure described for Example 49 above, additional compounds described herein were prepared by substituting the appropriate amine in step a and/or boronic ester starting material in step b, suitable reagents and reaction conditions, obtaining compounds such as those selected from:

| Cpd ID | Data |
|---|---|
| 340 | Starting material: 2,8-dimethyl-6-(4,4,5,5-tetramethyl-1,3,2-dioxaborolan- |
| | 2-yl)imidazo[1,2-b]pyridazine |
| | MS: m/z 394.1 [M + H]⁺ ]⁺; RT: 0.9 min (Method 10) |
| 341 | Starting material: 2,8-dimethyl-6-(4,4,5,5-tetramethyl-1,3,2-dioxaborolan- |
| | 2-yl)imidazo[1,2-b]pyridazine |
| | MS: m/z 433.49 [M + H]⁺ ]⁺; RT: 1.923 min (Method 8) |
| 342 | Starting material: 1-tert-butylazetidin-3-amine |
| | MS: m/z 436.2 [M + H]⁺ ]⁺; RT: 1.382 min (Method 8) |
| 343 | Starting material:tert-butyl 3-(methylamino)azetidine-1-carboxylate |
| | MS: m/z 436.2 [M + H]⁺ ]⁺; RT: 0.563 min (Method 7) |
| 344 | Starting material: N,2,2,6,6-pentamethylpiperidin-4-amine |
| | MS: m/z 478.3 [M + H]⁺ ]⁺; RT: 1.00 min (Method 3) |
| 345 | Starting material: 2,2,6,6-tetramethylpiperidin-4-amine |
| | MS: m/z 464.3 [M + H]⁺ ]⁺; RT: 1.00 min (Method 3) |
| 346 | Starting material: 2-bromo-4,6-dimethyl-pyrazolo[1,5-a]pyrazine |
| | MS: m/z 422.2 [M + H]⁺ ]⁺; RT: 1.316 min (Method 10) |
| 347 | Starting material: (1S,5S)-3-methyl-3,6-diazabicyclo[3.2.0]heptane |
| | dihydrochloride |
| | MS: m/z 420.2 [M + H]⁺ ]⁺; RT: 0.86 min (Method 3) |

### Example 49—Compound 338

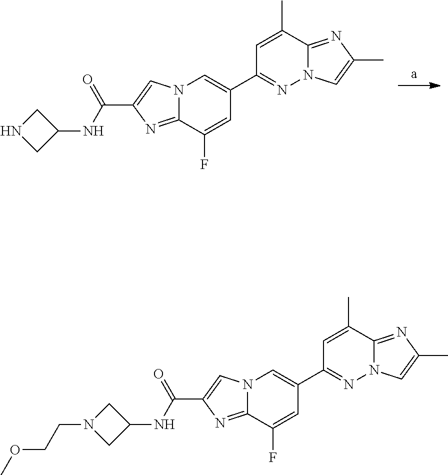

Step a: To a solution of N-(azetidin-3-yl)-6-(2,8-dimethylimidazo[1,2-b]pyridazin-6-yl)-8-fluoro-imidazo[1,2-a]pyridine-2-carboxamide (20 mg, 52.72 μmol), K₂CO₃ (14.57 mg, 105.43 mol) in DMF (5 mL) was added 1-iodo-2-methoxy-ethane (9.80 mg, 52.72 μmol). The reaction was stirred at 60° C. for 12 h. The mixture was filtered and concentrated under reduced pressure to give a residue, which was purified by Pre-HPLC to give 6-(2,8-dimethylimidazo[1,2-b]pyridazin-6-yl)-8-fluoro-N-[1-(2-methoxyethyl)azetidin-3-yl]imidazo[1,2-a]pyridine-2-carboxamide (3.38 mg, 7.04 μmol, 13.3% yield) as a yellow solid. MS: m/z 438.2 [M+H]⁺; RT: 1.43 min (method 8).

### Example 50—Compound 208

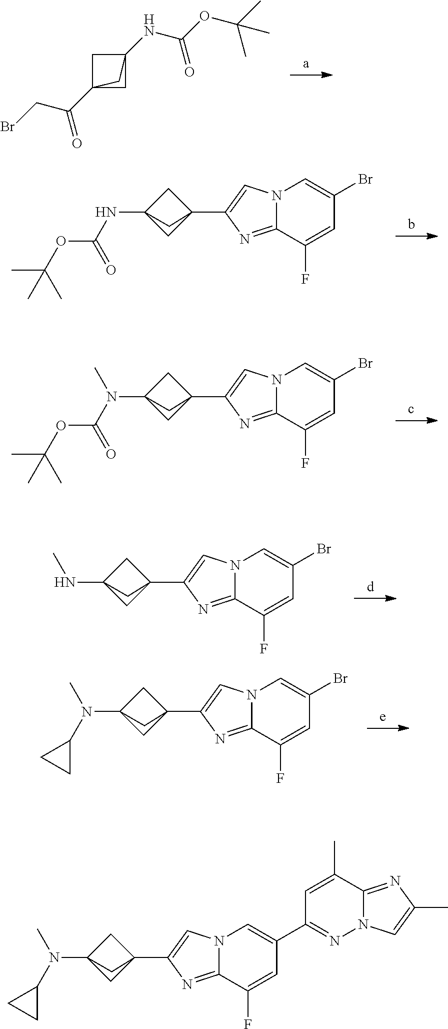

Step a: To a solution of tert-butyl N-[3-(2-bromoacetyl)-1-bicyclo[1.1.1]pentanyl]carbamate (220 mg, 723.26 umol, 1.0 eq.) and 5-bromo-3-fluoro-pyridin-2-amine (165.77 mg, 867.91 umol, 1.2 eq.) in 2-methylpropan-2-ol (5 mL, 0.14M) was added NaHCO3 (121.52 mg, 1.45 mmol, 2.0 eq.). The reaction mixture was stirred at 80° C. for 12 h. The reaction mixture was filtered and concentrated under reduced pressure to give a residue. The residue was purified by flash silica gel chromatography (from PE/EtOAc=6/1 to 3/1, TLC: PE/EtOAc=3/1, Rf=0.3) to yield tert-butyl N-[3-(6-bromo-8-fluoro-imidazo[1,2-a]pyridin-2-yl)-1-bicyclo[1.1.1]pentanyl]carbamate (40 mg, 98.70 umol, 27.29% yield, 97.78% purity) as a white solid. MS: m/z 398.0 [M+H]⁺; RT: 0.406 min (Method 9).

Step b: To a solution of tert-butyl N-[3-(6-bromo-8-fluoro-imidazo[1,2-a]pyridin-2-yl)-1-bicyclo[1.1.1]pentanyl]carbamate (100 mg, 252.36 umol, 1.0 eq.) in THF (3 mL) was added NaH (18.17 mg, 757.09 umol, 3.0 eq.) and MeI (70.75 mg, 504.73 umol, 2.0 eq.) at 0° C. The reaction was stirred at 20° C. for 3 h. The reaction mixture was diluted with H2O (20 mL) and extracted with EtOAc (20 mL×3), dried over Na₂SO₄, filtered and concentrated under reduced pressure to give the tert-butyl N-[3-(6-bromo-8-fluoro-imidazo[1,2-a]pyridin-2-yl)-1-bicyclo[1.1.1]pentanyl]-N-methyl-carbamate (100 mg, 209.04 umol, 82.83% yield) as a yellow solid. MS: m/z 410.0 [M+H]⁺; RT: 0.543 min (Method 9).

Step c: To a solution of tert-butyl N-[3-(6-bromo-8-fluoro-imidazo[1,2-a]pyridin-2-yl)-1-bicyclo[1.1.1]pentanyl]-N-methyl-carbamate (100 mg, 243.74 umol, 1.0 eq.) in EtOAc (3 mL, 0.04M) was added HCl/EtOAC (3 mL, 0.04M). The reaction mixture was stirred at 20° C. for 1 h. The reaction mixture was filtered and concentrated under reduced pressure to give 3-(6-bromo-8-fluoro-imidazo[1,2-a]pyridin-2-yl)-N-methyl-bicyclo[1.1.1]pentan-1-amine (60 mg, 193.45 umol, 79.37% yield) as a yellow solid. MS: m/z 312.0 [M+H]⁺; RT: 0.251 min (Method 9).

Step d: To a solution of 3-(6-bromo-8-fluoro-imidazo[1,2-a]pyridin-2-yl)-N-methyl-bicyclo[1.1.1]pentan-1-amine (40 mg, 128.96 umol, 1.0 eq.) in MeOH (2 mL, 0.064M) was added (1-ethoxycyclopropoxy)-trimethyl-silane (33.72 mg, 193.45 umol, 1.5 eq.), TEA (13.05 mg, 128.96 umol, 1.0 eq.) and sodium; cyanoboranuide (12.97 mg, 206.34 umol, 1.6 eq.) and stirred at 20° C. for 20 min. Then acetic acid (23.23 mg, 386.89 umol, 3.0 eq.) was added and the mixture stirred at 60° C. for 16 h. The reaction mixture was diluted with H2O (50 mL) and extracted with EtOAc (50 mL×3). The combined organic layers were dried over Na2SO4, filtered and concentrated under reduced pressure to give a residue, which was purified by prep-HPLC (FA condition) to give the 3-(6-bromo-8-fluoro-imidazo[1,2-a]pyridin-2-yl)-N-cyclopropyl-N-methyl-bicyclo[1.1.1]pentan-1-amine (35 mg, 99.51 umol, 77.16% yield) as yellow oil. MS: m/z 349.9 [M+H]⁺; RT: 0.241 min (Method 9).

Step e: To a solution of 2,8-dimethyl-6-(4,4,5,5-tetramethyl-1,3,2-dioxaborolan-2-yl)imidazo[1,2-b]pyridazine (27.30 mg, 99.93 umol, 1.0 eq.) and 3-(6-bromo-8-fluoro-imidazo[1,2-a]pyridin-2-yl)-N-cyclopropyl-N-methyl-bicyclo[1.1.1]pentan-1-amine (35 mg, 99.93 umol, 1.0 eq.) in Dioxane/H2O (5 mL, 0.02M) was added cyclopentyl(diphenyl)phosphane; dichloropalladium; iron (7.31 mg, 9.99 umol, 0.1 eq.) and K2CO3 (41.43 mg, 299.80 umol, 3.0 eq.) under N2. The reaction mixture was stirred at 90° C. for 2 hours. The reaction was filtered and concentrated under reduced pressure to give the residue. The residue was purified by preparative HPLC (neutral condition) to give the N-cyclopropyl-3-[6-(2,8-dimethylimidazo[1,2-b]pyridazin-6-yl)-8-fluoro-imidazo[1,2-a]pyridin-2-yl]-N-methyl-bicyclo[1.1.1]pentan-1-amine (13.1 mg, 30.80 umol, 30.82% yield) as a yellow solid. MS: m/z 417.2 [M+H]⁺; RT: 1.02 min (Method 8).

### Example 51—Compound 335

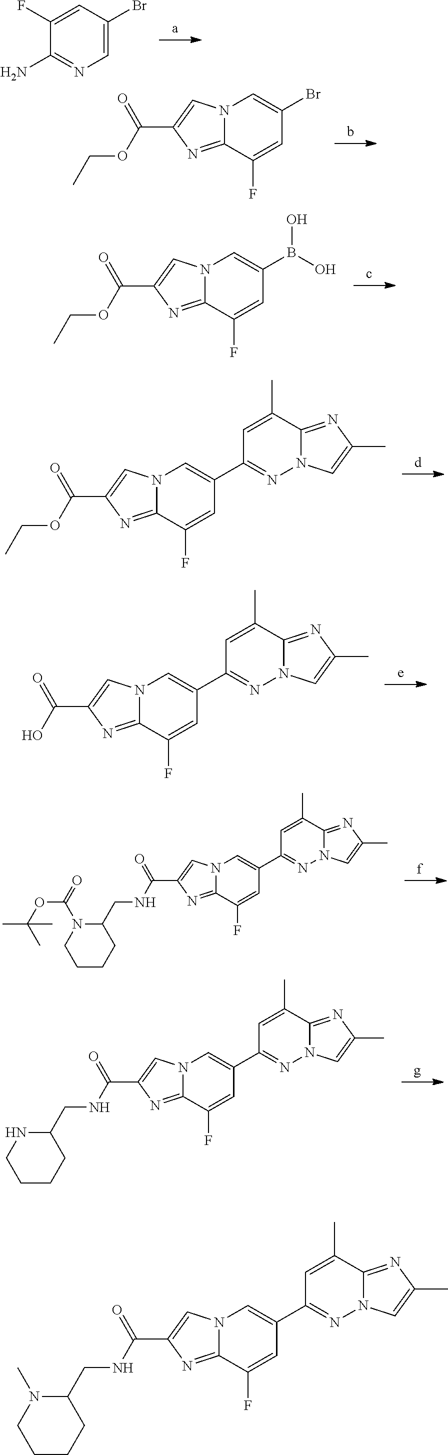

Step a: To a solution of 5-bromo-3-fluoropyridin-2-amine (25 g, 130.89 mmol, 1.0 eq.) in EtOH (300 mL, 0.44M) was added ethyl 3-bromo-2-oxopropanoate (25.52 g, 130.89 mmol, 1.0 eq.). The mixture was stirred at 78° C. for 16 hr. The reaction mixture was concentrated under reduced pressure. The residue was washed with saturated NaHCO3 (400 mL*1) and extracted with (3×500 mL). The organic layer was dried over Na2SO4, filtered and concentrated under reduced pressure to give a residue. The crude material was purified on silica gel column chromatography (from pure DCM to DCM/MeOH=10/1, TLC:PE/EA=5/1, Rf=0.2) to give ethyl 6-bromo-8-fluoro-imidazo[1,2-a]pyridine-2-carboxylate (15 g, 52.25 mmol, 39.92% yield) as a white solid. MS: m/z 288.9 [M+H]⁺; RT: 0.37 min (Method 9).

Step b: To a solution of ethyl 6-bromo-8-fluoro-imidazo[1,2-a]pyridine-2-carboxylate (800 mg, 2.79 mmol, 1.0 eq.) and 4,4,5,5-tetramethyl-2-(4,4,5,5-tetramethyl-1,3,2-dioxaborolan-2-yl)-1,3,2-dioxaborolane (849.16 mg, 3.34 mmol, 1.2 eq.) in dioxane (10 mL, 0.28M) was added KOAc (546.96 mg, 5.57 mmol, 2.0 eq.) and Pd(dppf)Cl2 (203.90 mg, 278.66 umol, 0.1 eq.) under N2. The reaction mixture was stirred at 90° C. for 2 h. The reaction mixture was filtered and concentrated under reduced pressure to give (2-ethoxycarbonyl-8-fluoro-imidazo[1,2-a]pyridin-6-yl)boronic acid (600 mg, crude, 85% yield) as a black solid. MS: m/z 253.0 [M+H]⁺; RT: 0.26 min (Method 9).

Step c: To a solution of (2-ethoxycarbonyl-8-fluoro-imidazo[1,2-a]pyridin-6-yl)boronic acid (600 mg, 2.38 mmol, 1.0 eq.) and 6-chloro-2,8-dimethyl-imidazo[1,2-b]pyridazine (432.42 mg, 2.38 mmol, 1.0 eq.) in Dioxane (5 mL, 0.4M) and H2O (1 mL, 0.4M) was added K2CO3 (987.19 mg, 7.14 mmol, 3.0 eq.) and Pd(dppf)Cl2 (174.21 mg, 238.09 umol, 0.1 eq.) under N2. The reaction mixture was stirred at 90° C. for 1 h. The reaction mixture was filtered and concenrated under reduce pressure to give a residue, which was purified by flash silica gel chromatography (from PE/EtOAc=10/1 to 5/1, TLC: PE/EtOAc=3/1, Rf=0.25) to give ethyl 6-(2,8-dimethylimidazo[1,2-b]pyridazin-6-yl)-8-fluoro-imidazo[1,2-a]pyridine-2-carboxylate (600 mg, 1.25 mmol, 52.59% yield) as yellow solid. MS: m/z 354.0 [M+H]⁺; RT: 0.29 min (Method 9).

Step d: To a solution of ethyl 6-(2,8-dimethylimidazo[1,2-b]pyridazin-6-yl)-8-fluoro-imidazo[1,2-a]pyridine-2-carboxylate (300 mg, 849.02 umol, 1.0 eq.) in MeOH (2 mL, 0.34M) was added NaOH (101.88 mg, 2.55 mmol, 0.3 eq.) in H2O (0.5 mL, 0.34). The reaction mixture was stirred at 20° C. for 12 h. The reaction mixture was quenched by addition 1 N HCl at 0° C. until pH=5 and extracted with EtOAc (30 mL×3). The combined organic layers were washed with brine (30 mL), dried over Na₂SO₄, filtered and concentrated under reduced pressure to give 6-(2,8-dimethylimidazo[1,2-b]pyridazin-6-yl)-8-fluoro-imidazo[1,2-a]pyridine-2-carboxylic acid (270 mg, crude, 98% yield) as a yellow solid. MS: m/z 326.0 [M+H]⁺; RT: 0.33 min (Method 9).

Step e: To a solution of 6-(2,8-dimethylimidazo[1,2-b]pyridazin-6-yl)-8-fluoro-imidazo[1,2-a]pyridine-2-carboxylic acid (40 mg, 122.96 umol, 1.0 eq.) in Pyridine (5 mL, 0.025M) was added tert-butyl-2-(aminomethyl)piperidine-1-carboxylate (52.70 mg, 245.93 umol, 2.0 eq.) and EDCl (28.29 mg, 147.56 umol, 1.2 eq.). The reaction mixture was stirred at 80° C. for 12 h. The mixture was filtered and concentrated to give a residue. The reaction mixture was diluted with H2O (10 mL) and extracted with EtOAc (10 mL×3). The combined organic layers were washed with brine (10 mL), dried over Na₂SO₄, filtered and concentrated under reduced pressure to give a residue. The residue was purified by preparative TLC (DCM/MeOH=10/1, TLC: DCM/MeOH=10/1, Rf=0.30). tert-butyl (2R)-2-[[[6-(2,8-dimethylimidazo[1,2-b]pyridazin-6-yl)-8-fluoro-imidazo[1,2-a]pyridine-2-carbonyl]amino]methyl]piperidine-1-carboxylate (25 mg, 42.66 umol, 34.69% yield) was obtained as a yellow solid. MS: m/z 522.4 [M+H]⁺; RT: 0.48 min (Method 9).

Step f: To a solution of tert-butyl (2R)-2-[[[6-(2,8-dimethylimidazo[1,2-b]pyridazin-6-yl)-8-fluoro-imidazo[1,2-a]pyridine-2-carbonyl]amino]methyl]piperidine-1-carboxylate (25 mg, 47.93 umol, 1.0 eq.) in HCl/EtOAc (3 mL, 0.016M) was stirred at 25° C. for 1 h. The mixture was filtered and concentrated to give a residue. The crude compound was used into the next step without further purification. 6-(2,8-dimethylimidazo[1,2-b]pyridazin-6-yl)-8-fluoro-N-[[(2R)-2-piperidyl]methyl]imidazo[1,2-a]pyridine-2-carboxamide (15 mg, 34.52 umol, 72.02% yield) was obtained as a yellow solid. MS: m/z 422.2 [M+H]⁺; RT: 0.28 min (Method 9).

Step g: To a solution 6-(2,8-dimethylimidazo[1,2-b]pyridazin-6-yl)-8-fluoro-N-[[(2R)-2-piperidyl]methyl]imidazo[1,2-a]pyridine-2-carboxamide (15 mg, 35.59 umol, 1.0 eq.) in DCE/EtOH (3 mL, 0.012M) was added Paraformaldehyde (64.04 mg, 53.38 umol, 1.5 eq.) and acetic acid (6.41 mg, 106.77 umol, 3.0 eq.). The reaction mixture was stirred 25° C. for 10 mins then was added sodium; triacetoxyboranuide (11.31 mg, 53.38 umol, 1.5 eq.), the reaction mixture was stirred at 25° C. for 12 h. The mixture was filtered and concentrated to give a residue. The residue was purified by prep-HPLC (NH4HCO3 condition). 6-(2,8-dimethylimidazo[1,2-b]pyridazin-6-yl)-8-fluoro-N-[[(2R)-1-methyl-2-piperidyl]methyl]imidazo[1,2-a]pyridine-2-carboxamide (3.5 mg, 7.70 umol, 21.64% yield) was obtained as a yellow solid. MS: m/z 436.2 [M+H]⁺; RT: 2.51 min (Method 8).

| | |
|---|---|
| 333 | Starting material: tert-butyl N-(3-aminocyclopentyl)carbamate |
| | MS: m/z 436.3 [M + H]⁺; RT: 1.15 min (Method 10) |

### Example 52—Compound 540

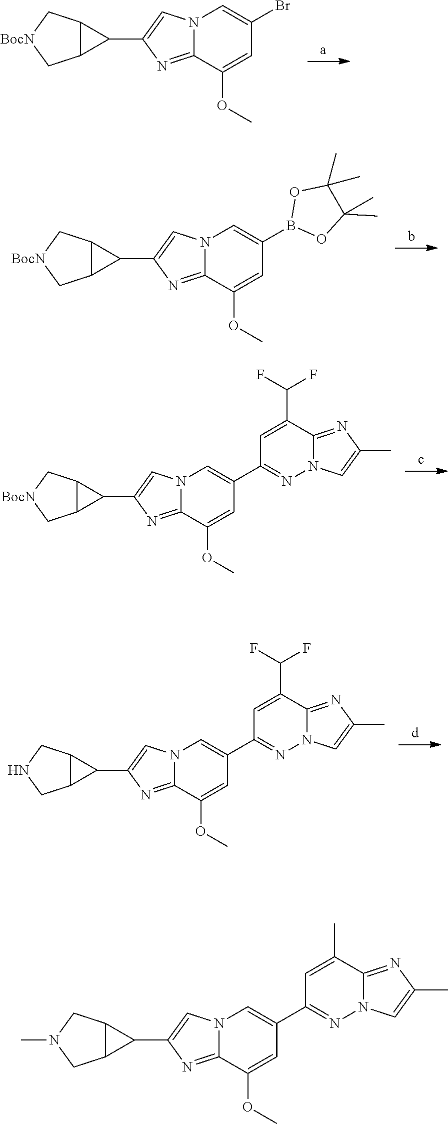

Step a: To a solution of tert-butyl-6-(6-bromo-8-methoxy-imidazo[1,2-a]pyridin-2-yl)-3-azabicyclo[3.1.0]hexane-3-carboxylate (190 mg, 465.36 umol, 1.0 eq.) in Dioxane (5 mL, 0.093M) was added KOAc (137.01 mg, 1.40 mmol, 3.0 eq.), 4,4,5,5-tetramethyl-2-(4,4,5,5-tetramethyl-1,3,2-dioxaborolan-2-yl)-1,3,2-dioxaborolane (129.99 mg, 511.89 umol, 1.1 eq.) and cyclopentyl(diphenyl)phosphane; dichloropalladium; iron (68.10 mg, 93.07 umol, 0.2 eq.). The mixture was stirred at 90° C. for 12 h under N2. The crude compound was used into the next step without further purification. MS: m/z 455.9 [M+H]⁺; RT: 0.56 min (Method 9).

Step b: To a solution of tert-butyl-6-[8-methoxy-6-(4,4,5,5-tetramethyl-1,3,2-dioxaborolan-2-yl)imidazo[1,2-a]pyridin-2-yl]-3-azabicyclo[3.1.0]hexane-3-carboxylate (100 mg, 219.61 umol, 1.0 eq.) in Dioxane (5 mL, 0.037M) and H2O (1 mL 0.037M) was added K2CO3 (91.05 mg, 658.83 umol, 3.0 eq.), 6-chloro-8-(difluoromethyl)-2-methyl-imidazo[1,2-b]pyridazine (47.79 mg, 219.61 umol, 1.0 eq.) and cyclopentyl(diphenyl)phosphane; dichloropalladium; iron (32.14 mg, 43.92 umol, 0.2 eq.). The mixture was stirred at 90° C. for 2 h under N2. The mixture was filtered and concentrated to give a residue. The residue was purified by prep-HPLC purification (basic condition). Compound tert-butyl-6-[6-(4,6-dimethylpyrazolo[1,5-a]pyrazin-2-yl)-8-methoxy-imidazo[1,2-a]pyridin-2-yl]-3-azabicyclo[3.1.0]hexane-3-carboxylate (19 mg, 40.04 umol, 18.23% yield). MS: m/z 511.3 [M+H]⁺; RT: 0.38 min (Method 9).

Step c: To a solution of tert-butyl-6-[6-[8-(difluoromethyl)-2-methyl-imidazo[1,2-b]pyridazin-6-yl]-8-methoxy-imidazo[1,2-a]pyridin-2-yl]-3-azabicyclo[3.1.0]hexane-3-carboxylate (12 mg, 23.50 umol, 1.0 eq.) in HCl/EtOAc (2 mL, 0.012M). The mixture was stirred at 25° C. for 8 h. The mixture was filtered and concentrated to give a residue. The crude compound was used into the next step without further purification. Compound 6-[2-[(1S,5R)-3-azabicyclo[3.1.0]hexan-6-yl]-8-methoxy-imidazo[1,2-a]pyridin-6-yl]-8-(difluoromethyl)-2-methyl-imidazo[1,2-b]pyridazine (8 mg, 19.49 umol, 82.93% yield). MS: m/z 411.1 [M+H]⁺; RT: 0.24 min (Method 9).

Step d: To a solution of 6-[2-[3-azabicyclo[3.1.0]hexan-6-yl]-8-methoxy-imidazo[1,2-a]pyridin-6-yl]-8-(difluoromethyl)-2-methyl-imidazo[1,2-b]pyridazine (8 mg, 19.49 umol, 1.0 eq.) in DCE/EtOH (4 mL, 0.004M) was added Paraformaldehyde (23.38 mg, 19.49 umol, 1.0 eq.). The mixture was stirred at 25° C. for 10 min. Then added sodium; triacetoxyboranuide (12.39 mg, 58.48 umol, 3.0 eq.). The mixture was stirred at 25° C. for 2h. LCMS. The mixture was filtered and concentrated to give a residue. The residue was purified by prep-HPLC purification (basic condition). Compound 8-(difluoromethyl)-6-[8-methoxy-2-[3-methyl-3-azabicyclo[3.1.0]hexan-6-yl]imidazo[1,2-a]pyridin-6-yl]-2-methyl-imidazo[1,2-b]pyridazine (6.71 mg, 15.81 umol, 81.10% yield) was obtained as white solid. MS: m/z 425.2 [M+H]⁺; RT: 0.75 min (Method 10).

Using the procedure described for Example 52 above, additional compounds described herein were prepared by substituting the appropriate boronic ester starting material in step b, suitable reagents and reaction conditions, obtaining compounds such as those selected from:

| | |
|---|---|
| 541 | Starting material: 6-chloro-2,8-dimethyl-[1,2,4]triazolo[1,5-b]pyridazine |
| | MS: m/z 390.1 [M + H]⁺ ]⁺; RT: 0.66 min (Method 10) |
| 542 | Starting material: 2-bromo-4,6-dimethyl-pyrazolo[1,5-a]pyrazine |
| | MS: m/z 389.2 [M + H]⁺ ]⁺; RT: 0.68 min (Method 10) |

### Example 53—Compound 290

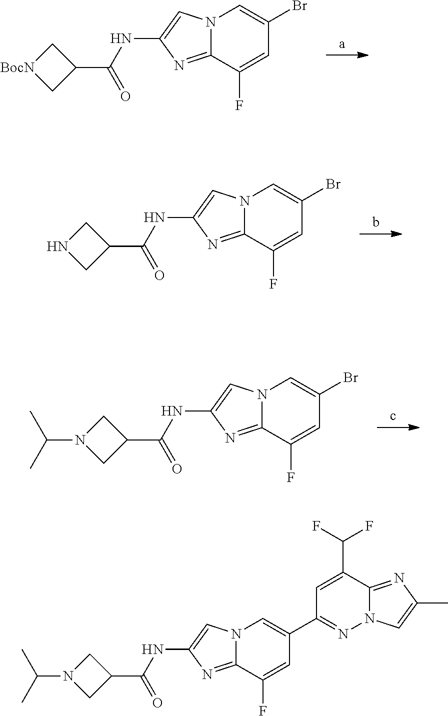 [SMILES: CC(C)(C)OC(=O)N1CC(C(=O)Nc2cn3cc(Br)cc(F)c3n2)C1 ; O=C(Nc1cn2cc(Br)cc(F)c2n1)C1CNC1 ; CC(C)N1CC(C(=O)Nc2cn3cc(Br)cc(F)c3n2)C1 ; Cc1cn2nc(-c3cc(F)c4nc(NC(=O)C5CN(C(C)C)C5)cn4c3)cc(C(F)F)c2n1]

Step a: The mixture of tert-butyl 3-[(6-bromo-8-fluoro-imidazo[1,2-a]pyridin-2-yl)carbamoyl]azetidine-1-carboxylate (60 mg, 145.19 umol, 1.0 eq.) and TFA (1.49 g, 13.06 mmol, 90 eq.) in DCM (3 mL 0.05M) was stirred at 25° C. for 1 hours. The reaction mixture was concentrated in vacuum to give crude product. The crude product was purified by prep-HPLC to give N-(6-bromo-8-fluoro-imidazo[1,2-a]pyridin-2-yl)azetidine-3-carboxamide (30 mg, 95.81 μmol, 65.99% yield) as a yellow solid. MS: m/z 313.0 [M+H]⁺; RT: 1.72 min (Method 10).

Step b: To a solution of N-(6-bromo-8-fluoro-imidazo[1,2-a]pyridin-2-yl)azetidine-3-carboxamide (30 mg, 95.81 umol, 1.0 eq.) in EtOH (3 mL, 0.032M) was added acetone (5.56 mg, 95.81 umol, 1.0 eq.), DCE (18.96 mg, 191.62 umol, 2.0 eq.) and acetic acid (17.26 mg, 287.42 umol, 3.0 eq.) and the mixture was stirred at 25° C. for 20 min. sodium; triacetoxyboranuide (32.49 mg, 153.29 umol, 1.6 eq.) was added and the mixture was stirred at 25° C. for 1 h. The mixture was filtered and the filtrate was concentrated in vacuo to give a residue. The crude product was purified by prep-HPLC to give N-(6-bromo-8-fluoro-imidazo[1,2-a]pyridin-2-yl)-1-isopropyl-azetidine-3-carboxamide (30 mg, 84.46 μmol, 88.15% yield) as a yellow solid. MS: m/z 357.0 [M+H]⁺; RT: 0.94 min (Method 10)

Step c: To a solution of N-(6-bromo-8-fluoro-imidazo[1,2-a]pyridin-2-yl)-1-isopropyl-azetidine-3-carboxamide (10 mg, 28.15 μmol) and [8-(difluoromethyl)-2-methyl-imidazo[1,2-b]pyridazin-6-yl]boronic acid (12.78 mg, 56.31 μmol, 1.0 eq.) in Dioxane (3 mL, 0.007M) and water (1 mL, 0.007M) was added Pd(dppf)Cl2 (2.06 mg, 2.82 μmol, 0.1 eq.) and K2CO3 (7.78 mg, 56.31 μmol, 2.0 eq.). The mixture was stirred under nitrogen at 90° C. for 2 hours. The reaction mixture was concentrated in vacuum to give crude product. The crude product was purified by prep-HPLC (Column Boston Green ODS 150*30 mm*5 um; Condition: water (FA)-ACN, Begin B 8, End B 38; Gradient Time (min): 14; 100% B Hold Time (min): 2; Flow Rate (ml/min): 25) to give N-[6-[8-(difluoromethyl)-2-methyl-imidazo[1,2-b]pyridazin-6-yl]-8-fluoro-imidazo[1,2-a]pyridin-2-yl]-1-isopropyl-azetidine-3-carboxamide (3.3 mg, 7.21 μmol, 25.62% yield) as a yellow solid. MS: m/z 458.2 [M+H]⁺; RT: 1.12 min (Method 10)

Using the procedure described for Example 53 above, additional compounds described herein were prepared by substituting the appropriate amine in step a and/or boronic ester starting material in step c, suitable reagents and reaction conditions, obtaining compounds such as those selected from:

| Cpd ID | Data |
|---|---|
| 309 | Starting material: 8-(difluoromethyl)-2-methyl-6-(4,4,5,5-tetramethyl- |
| | 1,3,2-dioxaborolan-2-yl)imidazo[1,2-b]pyridazine |
| | MS: m/z 459.1 [M + H]⁺ ]⁺; RT: 1.81 min (Method 10) |
| 310 | Starting material: 2,8-dimethyl-6-(4,4,5,5-tetramethyl-1,3,2-dioxaborolan- |
| | 2-yl)-[1,2,4]triazolo[1,5-b]pyridazine |
| | MS: m/z 431.1 [M + H]⁺ ]⁺; RT: 1.763 min (Method 10) |

### Example 54—Compound 411

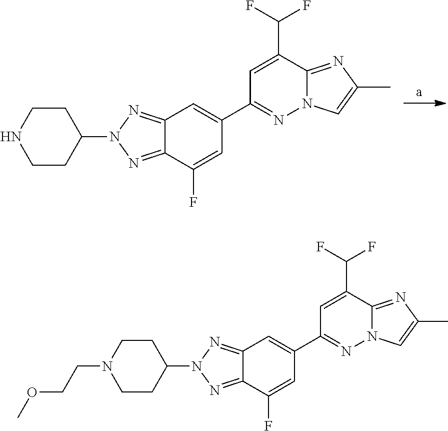

Step a: To a solution of 6-[8-(difluoromethyl)-2-methyl-imidazo[1,2-b]pyridazin-6-yl]-4-fluoro-2-(4-piperidyl)benzotriazole (20 mg, 49.83 μmol, 1.0 eq.) in MeCN (2 mL, 0.025M) was added K2CO3 (13.77 mg, 99.65 μmol, 2.0 eq.) and 1-bromo-2-methoxy-ethane (6.93 mg, 49.83 μmol, 1.0 eq.). The reaction mixture was stirred at 60° C. for 16 h. The reaction mixture was filtered and concentrated under reduced pressure to give a residue, which was purified by Pre-HPLC (neutral conditions) to give the 6-[8-(difluoromethyl)-2-methyl-imidazo[1,2-b]pyridazin-6-yl]-4-fluoro-2-[1-(2-methoxyethyl)-4-piperidyl]benzotriazole (14.9 mg, 31.61 μmol, 63.44% yield) as a yellow solid. MS: m/z 460.1 [M+H]⁺; RT: 1.35 min (Method 8)

| | |
|---|---|
| 412 | Starting material: 1-bromo-3-methoxy-propane |
| | MS: m/z 474.2 [M + H]⁺ ]⁺; RT: 1.37 min (Method 8) |

### Example 55—Compound 457

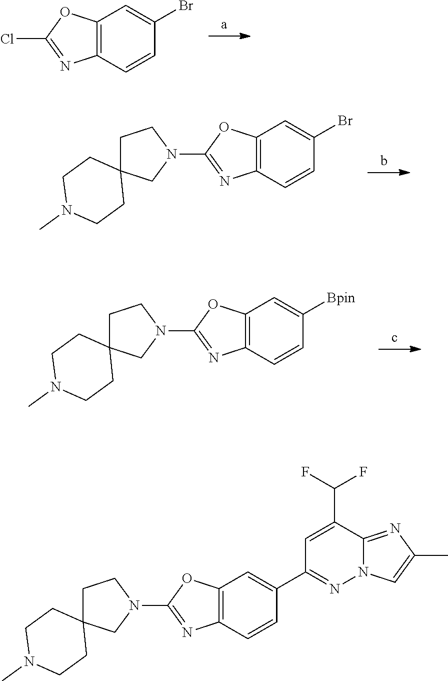

Step a: To a solution of 6-bromo-2-chloro-4-fluoro-1,3-benzoxazole (200 mg, 798.56 μmol) and 8-methyl-2,8-diazaspiro[4.5]decane (123.18 mg, 798.56 μmol) in dioxane (8 mL) was added TEA (242.42 mg, 2.40 mmol, 333.91 μL). The mixture was stirred at 100° C. for 2 hours. The reaction was filtered and concentrated. The residue was purified by flash silica gel chromatography (from MeOH/EtOAc=0/100 to 20/80) to yield the product as a yellow solid (250 mg, 678.9 μmol, 85% yield). MS: m/z 369.8 [M+H]⁺.

Step b: To a solution of 6-bromo-4-fluoro-2-(8-methyl-2,8-diazaspiro[4.5]decan-2-yl)-1,3-benzoxazole (200 mg, 543.12 μmol) and 4,4,5,5-tetramethyl-2-(4,4,5,5-tetramethyl-1,3,2-dioxaborolan-2-yl)-1,3,2-dioxaborolane (179.29 mg, 706.06 μmol) in dioxane (20 mL) was added KOAc (159.91 mg, 1.63 mmol) and Pd(dppf)Cl₂ (39.74 mg, 54.31 μmol). The mixture was stirred under nitrogen at 90° C. for 2 hours. The crude compound was used into the next step without further purification.

Step c: To a solution of 6-chloro-8-(difluoromethyl)-2-methyl-imidazo[1,2-b]pyridazine (30.67 mg, 140.95 μmol) and 2-(8-methyl-2,8-diazaspiro[4.5]decan-2-yl)-6-(4,4,5,5-tetramethyl-1,3,2-dioxaborolan-2-yl)-1,3-benzoxazole (56 mg, 140.95 μmol) in dioxane (3 mL) and water (1 mL) was added K₂CO₃ (38.96 mg, 281.89 μmol) and [1,1′-Bis(diphenylphosphino)ferrocene]dichloropalladium (20.63 mg, 28.19 μmol). The mixture was stirred under nitrogen at 90° C. for 2 hours. The reaction mixture was concentrated in vacuum. The residue was purified by prep-HPLC (Column: Boston Green ODS 150*30 mm*5 um; Condition: water (FA)-ACN, Begin B 16, End B 46; Gradient Time (min): 18; 100% B Hold Time (min): 2; Flow Rate (ml/min): 25) to give 6-[8-(difluoromethyl)-2-methyl-imidazo[1,2-b]pyridazin-6-yl]-2-(8-methyl-2,8-diazaspiro[4.5]decan-2-yl)-1,3-benzoxazole (7.6 mg, 16.80 μmol, 11.92% yield, 100% purity) as a yellow solid. MS: m/z 453.3 [M+H]⁺; RT: 1.403 min (Method 10)

### Example 56—Compound 531

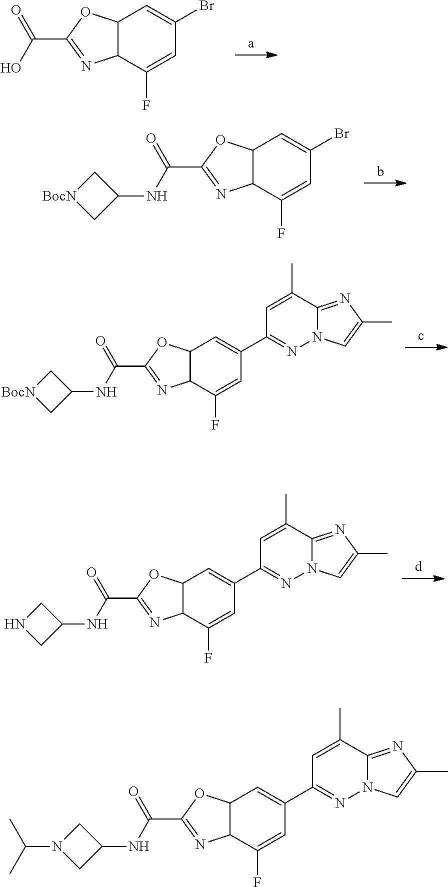

Step a: To a solution of 6-bromo-4-fluoro-1,3-benzoxazole-2-carboxylic acid (100 mg, 384.59 μmol) in pyridine (1 mL) was added tert-butyl 3-aminoazetidine-1-carboxylate (79.48 mg, 461.51 μmol) and EDCI (147.45 mg, 769.18 μmol). The reaction was stirred at 90° C. for 1 h. Then the mixture was concentrated, and water (80 mL) was added. The residue was extracted with EtOAc (50 mL×3). The combined organic layers were washed with brine (50 mL), dried over Na₂SO₄, filtered and concentrated in vacuo to give the residue, which was purified by Combi-Flash (DCM/MeOH=10/1) to give tert-butyl 3-[(6-bromo-4-fluoro-1,3-benzoxazole-2-carbonyl)amino]azetidine-1-carboxylate (100 mg, 241.41 μmol, 62.77% yield) as yellow solid. MS: m/z 417.0 [M+H]⁺.

Step b: To a solution of tert-butyl 3-[(6-bromo-4-fluoro-1,3-benzoxazole-2-carbonyl)amino]azetidine-1-carboxylate (30 mg, 72.42 μmol) in dioxane (5 mL) and water (0.5 mL) was added 2,8-dimethyl-6-(4,4,5,5-tetramethyl-1,3,2-dioxaborolan-2-yl)imidazo[1,2-b]pyridazine (19.78 mg, 72.42 μmol), Pd(dppf)Cl₂ (5.30 mg, 7.24 μmol) and K₂CO₃ (30.03 mg, 217.27 μmol) under N₂. The reaction was stirred at 90° C. for 2 h. The mixture was concentrated and then water (80 mL) was added. The residue was extracted with EtOAc (50 mL×3). The combined organic layers were washed with brine (50 mL), dried over Na₂SO₄, filtered, and concentrated in vacuo to give the crude product. The crude product was purified by prep-HPLC (FA) to yield tert-butyl 3-[[6-(2,8-dimethylimidazo[1,2-b]pyridazin-6-yl)-4-fluoro-1,3-benzoxazole-2-carbonyl]amino]azetidine-1-carboxylate (20 mg, 41.62 μmol, 57.47% yield) as brown solid. MS: m/z 481.2 [M+H]⁺.

Step c: To a solution of tert-butyl 3-[[6-(2,8-dimethylimidazo[1,2-b]pyridazin-6-yl)-4-fluoro-1,3-benzoxazole-2-carbonyl]amino]azetidine-1-carboxylate (10 mg, 20.81 μmol) in TFA (1 mL) was added DCM (2 mL). The mixture was stirred at 20° C. for 1 h and then concentrated in vacuo to give N-(azetidin-3-yl)-6-(2,8-dimethylimidazo[1,2-b]pyridazin-6-yl)-4-fluoro-1,3-benzoxazole-2-carboxamide (10 mg, crude) as yellow solid. MS: m/z 381.1 [M+H]⁺.

Step d: To a solution of N-(azetidin-3-yl)-6-(2,8-dimethylimidazo[1,2-b]pyridazin-6-yl)-4-fluoro-1,3-benzoxazole-2-carboxamide (10 mg, 26.29 μmol) in EtOH (2 mL) and DCE (0.5 mL) was added TEA (5.32 mg, 52.58 μmol, 7.33 μL) and acetone (1.53 mg, 26.29 μmol, 1.93 μL). The reaction was stirred at 20° C. for 10 minutes. Then sodium triacetoxyborohydride (11.14 mg, 52.58 μmol) was added and the reaction was further stirred at 20° C. for 0.5h. The mixture was filtered and the filtrate was concentrated in vacuo to give the residue, which was purified by prep-HPLC (FA) to give 6-(2,8-dimethylimidazo[1,2-b]pyridazin-6-yl)-4-fluoro-N-(1-isopropylazetidin-3-yl)-1,3-benzoxazole-2-carboxamide (1 mg, 2.37 μmol, 9.00% yield) as white solid. MS: m/z 423.2 [M+H]⁺; RT: 0.92 min (Method 10).

### Example 57—Compound 245

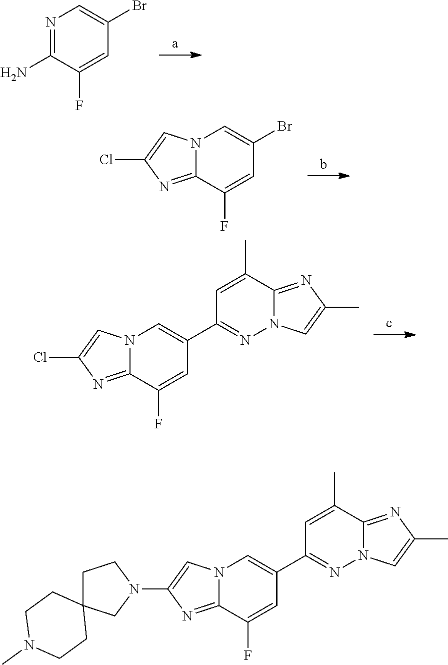

Step a: 5-bromo-3-fluoro-pyridin-2-amine (1 g, 5.24 mmol) and ethyl 2-bromoacetate (2.62 g, 15.71 mmol, 1.74 mL) were added to a 20 mL microwave vial. The reaction was stirred at 90° C. for 3 h. The resultant pinkish solid was filtered and added to another 20 mL microwave vial, followed by addition of phosphoryl trichloride (16.06 g, 104.71 mmol, 9.76 mL). The mixture was stirred at 90° C. overnight. The residue was transferred to a 500 mL round bottom flask. Methanol was added slowly to quench POCl₃ until no gas evolution, and the resultant suspension was concentrated down, redissolved in heptanes and filtered to give 6-bromo-2-chloro-8-fluoro-imidazo[1,2-a]pyridine (1.33 g, 5.34 mmol, 100% yield) as a yellow solid. MS: m/z 250.9 [M+H]⁺.

Step b: A 20 mL microwave vial was charged with Pd(dppf)Cl₂ (130.94 mg, 160.34 μmol) and 2,8-dimethyl-6-(4,4,5,5-tetramethyl-1,3,2-dioxaborolan-2-yl)imidazo[1,2-b]pyridazine (481.75 mg, 1.76 mmol), evacuated under vacuum and refilled with N₂ three times. A dioxane (4 mL) solution (purged with N₂) of 6-bromo-2-chloro-8-fluoro-imidazo[1,2-a]pyridine (400 mg, 1.60 mmol) and a water solution (purged) of dicesium carbonate (1.57 g, 4.81 mmol) were added to the vial under N₂. The mixture was stirred at 90° C. for 16 h. The residue was concentrated in V10 and purified with column chromatography (EA/hexane, 0 to 100% in 20 min) to give 6-(2-chloro-8-fluoro-imidazo[1,2-a]pyridin-6-yl)-2,8-dimethyl-imidazo[1,2-b]pyridazine (253 mg, 801.31 μmol, 49.98% yield) as a pale solid. MS: m/z 316.0 [M+H]⁺.

Step c: 6-(2-chloro-8-fluoro-imidazo[1,2-a]pyridin-6-yl)-2,8-dimethyl-imidazo[1,2-b]pyridazine (25 mg, 79.18 μmol), 8-methyl-2,8-diazaspiro[4.5]decane (30.20 mg, 158.36 μmol, Hydrochloride), (1E,4E)-1,5-diphenylpenta-1,4-dien-3-one; palladium (7.25 mg, 7.92 μmol) and sodium; 2-methylpropan-2-olate (45.66 mg, 475.09 μmol) were added in a microwave vial, evacuate under vacuum and refill with N2 three times, dioxane (1 mL) was then added. The reaction mixture was stirred at 90° C. for 12 h. The residue was concentrated in vacuo and purified with HPLC (Column: Sunfire C18 100×19 mm, 5 mm; Mobile phase A: MeCN; Mobile phase B: H2O; Modifier: 0.1% TFA) to give 2-[6-(2,8-dimethylimidazo[1,2-b]pyridazin-6-yl)-8-fluoro-imidazo[1,2-a]pyridin-2-yl]-8-methyl-2,8-diazaspiro[4.5]decane (1.5 mg, 2.74 μmol, 3.46% yield, Trifluoroacetic acid) as an orange solid. MS: m/z 434.3 [M+H]⁺; RT: 0.35 min (Method 4).

### Example 58—Compound 349

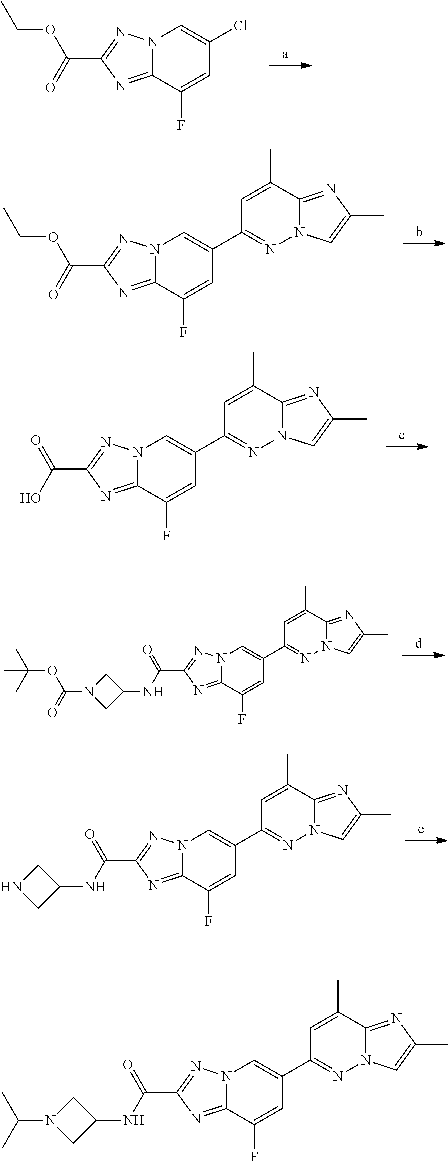

Step a: A mixture of ethyl 6-chloro-8-fluoro-[1,2,4]triazolo[1,5-a]pyridine-2-carboxylate (90 mg, 369.42 μmol, 1.0 eq.), 2,8-dimethyl-6-(4,4,5,5-tetramethyl-1,3,2-dioxaborolan-2-yl)imidazo[1,2-b]pyridazine (100.90 mg, 369.42 μmol, 1.0 eq.) In Dioxane (10 mL, 0.037M) was added Pd(dppf)Cl2 (27.03 mg, 36.94 μmol, 0.1 eq.), K2CO3 (153.17 mg, 1.11 mmol, 3.0 eq.) and stirred at 90° C. under N2 for 2 h. The reaction mixture was then quenched by water (50 mL). The aqueous layer was separated and extracted with EtOAc (30 mL×3). The combined organic layers were dried over Na₂SO₄, filtered and concentrated to afford crude product. The mixture was further purification by silica gel column chromatography (from PE/EtOAc=5/1 to 3/1,) to give ethyl 6-(2,8-dimethylimidazo[1,2-b]pyridazin-6-yl)-8-fluoro-[1,2,4]triazolo[1,5-a]pyridine-2-carboxylate (103 mg, 290.68 μmol, 78.69% yield) as a brown solid. MS: m/z 355.2 [M+H]⁺; RT: 1.58 min (Method 10).

Step b: A mixture of ethyl 6-(2,8-dimethylimidazo[1,2-b]pyridazin-6-yl)-8-fluoro-[1,2,4]triazolo[1,5-a]pyridine-2-carboxylate (100 mg, 282.22 μmol, 1.0 eq.) in NaOH (56.44 mg, 1.41 mmol, 5.0 eq.) in MeOH (8 mL, 0.018M) and H2O (8 mL, 0.018M) was stirred at 25° C. for 2 h. The reaction mixture was quenched by dilute hydrochloric acid (1 mol/L, 5 ml) and water (50 mL). The aqueous layer was separated and extracted with EtOAc (20 mL×3). The combined organic layers were dried over Na₂SO₄, filtered and concentrated to afford the crude product 6-(2,8-dimethylimidazo[1,2-b]pyridazin-6-yl)-8-fluoro-[1,2,4]triazolo[1,5-a]pyridine-2-carboxylic acid (90 mg, 275.83 μmol, 97.74% yield) as a white solid. MS: m/z 327.0 [M+H]⁺; RT: 1.05 min (Method 10).

Step c: To a mixture of 6-(2,8-dimethylimidazo[1,2-b]pyridazin-6-yl)-8-fluoro-[1,2,4]triazolo[1,5-a]pyridine-2-carboxylic acid (90 mg, 275.83 μmol, 1.0 eq.) and tert-butyl 3-aminoazetidine-1-carboxylate (47.51 mg, 275.83 μmol, 1.0 eq.) in Pyridine (5 mL, 0.055M) was added EDCI (158.63 mg, 827.50 μmol, 3.0 eq.) in one portion at 25° C. under N2. The mixture was stirred at 90° C. for 2 hours. The mixture was cooled to 25° C. and concentrated in reduced pressure. The residue was poured into water (50 mL). The aqueous phase was extracted with ethyl acetate (40 mL*3). The combined organic phase was washed with brine (50 mL), dried with anhydrous Na₂SO₄, filtered and concentrated in vacuum. The residue was purified by prep-HPLC (Column Boston Green ODS 150*30 mm*5 um; Condition water (FA)-ACN; Begin B 20; End B 50; Gradient Time (min) 12; 100% B Hold Time (min)2; FlowRate (ml/min) 25) to give tert-butyl 3-[[6-(2,8-dimethylimidazo[1,2-b]pyridazin-6-yl)-8-fluoro-[1,2,4]triazolo[1,5-a]pyridine-2-carbonyl]amino]azetidine-1-carboxylate (43 mg, 89.49 μmol, 32.44% yield) as a yellow solid. MS: m/z 481.2 [M+H]⁺; RT: 1.09 min (Method 10).

Step d: tert-butyl 3-[[6-(2,8-dimethylimidazo[1,2-b]pyridazin-6-yl)-8-fluoro-[1,2,4]triazolo[1,5-a]pyridine-2-carbonyl]amino]azetidine-1-carboxylate (40 mg, 83.25 μmol, 1.0 eq.) in DCM (3 mL, 0.028M) in one portion was added TFA (47.46 mg, 416.24 μmol, 5.0 eq.) at 25° C. The mixture was stirred for 16 hours. The reaction mixture was concentrated to afford the crude product N-(azetidin-3-yl)-6-(2,8-dimethylimidazo[1,2-b]pyridazin-6-yl)-8-fluoro-[1,2,4]triazolo[1,5-a]pyridine-2-carboxamide (30 mg, 78.87 μmol, 94.74% yield) as a yellow solid. The crude was used for next step directly. MS: m/z 381.2[M+H]⁺; RT: 0.89 min (Method 10).

Step e: A mixture of N-(azetidin-3-yl)-6-(2,8-dimethylimidazo[1,2-b]pyridazin-6-yl)-8-fluoro-[1,2,4]triazolo[1,5-a]pyridine-2-carboxamide (30 mg, 78.87 μmol, 1.0 eq.), acetone (22.90 mg, 394.34 μmol, 5.0 eq.) in EtOH (10 mL, 0.008M) was added TEA (39.90 mg, 394.34 μmol, 54.96 μL, 5.0 eq.). The solution then stirred for 5 minutes before acetic acid (23.68 mg, 394.34 μmol, 5.0 eq.) was added. After 30 mins, sodium; triacetoxyboranuide (50.15 mg, 236.61 μmol, 3.0 eq.) was added in one portion at 25° C. The mixture was stirred at 25° C. for 3 hours. The reaction mixture was purified by pre-HPLC (Column Welch Xtimate C18 150*25 mm*5 um; Condition water (FA)-ACN; Begin B 0; End B 30; Gradient Time (min) 11; 100% B Hold Time (min) 2; FlowRate (ml/min) 25) to give 6-(2,8-dimethylimidazo[1,2-b]pyridazin-6-yl)-8-fluoro-N-(1-isopropylazetidin-3-yl)-[1,2,4]triazolo[1,5-a]pyridine-2-carboxamide (17.2 mg, 40.71 μmol, 51.62% yield) as a white solid. MS: m/z 423.2 [M+H]⁺; RT: 1.64 min (Method 10).

### Section 3. Biological Assay and Data

HTT Mutant and Total HTRF iPSC Assay Protocol

The in-vitro cellular assay measures mutant and total Huntington (HTT) protein in human inducible pluripotent stem cells (iPSC) which were derived from a HTT patient with a poly-Q49 mutation. The assay measurement was performed by homogeneous time-resolved fluorescence (HTRF). The mutant HTT antibody is labeled with d-2 acceptor and recognizes an area in the poly Q region. The terbium (Tb) donor antibody recognizes a sequence at the N-terminus of the protein. The total HTT antibody was labeled with d2 acceptor and recognizes a sequence beyond the poly Q region. For each experiment run, a frozen aliquot of iPSC's was thawed from storage in liquid nitrogen and grown on Matrigel (Corning #354227) coated flasks using Complete Media ((mTeSR™ 1 Plus Basal Medium (STEMCELL Technologies cat #05825) supplemented with mTeSR™ 1 Plus (STEMCELL Technologies cat #05852) and penicillin/streptomycin (Gibco cat #10378016)) and in the presence of 10 uM Rock Inhibitor (Sigma #Y0503). The flask with cells was incubated overnight at 37° C. with 5% CO2 (Thermo) and next day the media was replaced with fresh Complete Media without Rock inhibitor and incubated for 48 hours for cell expansion at 37° C. with 5% CO2. Cells were harvested from flask using Accutase (Gibco #A1110501) and counted on Cellometer (Nexcelom Vision). A total of 10,000 cells/well were added in 30 ul volume of Complete Media with 10 uM rock inhibitor into a 384 well tissue culture plate (Perkin Elmer #NC1758152) pre-coated with Matrigel. The cell plate was centrifuged, and d cells were allowed to attach overnight at 37° C. and 5% CO2 in a high humidity incubator (Thermo Cytomat 10). The next day the cells were treated with compound. An intermediate plate was used to pre-dilute compounds in Complete Media with no rock inhibitor. Compounds were both diluted and dispensed using an ECHO (Labcyte #Echo555) into an empty 384 well PP plate (Griener #784201). A total of 60 ul of Complete Media was added per well using a multidrop Combi (Thermo #5840300). Compounds were tested in a 10 point, 3-fold titration, starting 10 uM. Media from cell plate was removed by flicking off media and plate was blotted on tissue paper. A volume of 50 uL was transferred from compound plate to the cell assay plate using an Integra (Viaflow384). Cell plate was incubated at 37° C., 5% CO2 with high humidity for 48 hours. Cell lysates were prepared by first removing media from plate and then adding 40 ul MPER lysis buffer (Thermo #78501) per well containing Protease and Phosphate inhibitor (Pierce #A32961). The plate was placed on an orbital shaker for 30 minutes at RT and an Apricot Dispenser (SPT Labtech) was used to transfer Sul of cell lysate into two 384 well black plates (Sigma Aldrich #CLS3821). Each plate contained either 5 ul/well of mutant or 5 ul/well of total HTT HTRF assay mix. The mutant HTT HTRF assay mix contained 2B7Ab-Tb “Donor” antibody (Thermo #CHDI-9000830) N-terminus labeled antibody at a final concentration 0.4 ng/well and MW1 (poly-Q specific)-d2 “acceptor” (Sigma #MABN2427) antibody final concentration 40 ng/well in HTRF Detection Buffer (CisBio #62SDBRDF). The total HTT HTRF assay mix contained 2B7Ab-Tb “Donor” N-terminus labeled antibody final concentration 0.4 ng/well and MAB2166-d2 (Anti-Huntingtin [1HU-4C8] mAb-d2 “acceptor” antibody with a final concentration 40 ng/well in HTRF Detection Buffer. All antibodies were labeled at Perkin Elmer. The assay plate was sealed and placed on an orbital shaker for a minute and then centrifuged for 1 minute before it was incubated at room temperature for 4 hours. The plate was read on a PHERAstar instrument (BMG LAB TECH) and HTRF ratio was calculated from (337 nm/665 nm) and (337 nm/620 nm) output. IC₅₀ values were generated from the full concentration-response curves. The curves were plotted as percent activity versus the compound concentration fitted to a variable 4-parameter logistic model.

A summary of IC₅₀ results is illustrated in Table 2, wherein “A” represents an IC₅₀ value of less than 100 nM, “B” represents an IC₅₀ value between 100 nM and 1 μM, and “C” represents an IC₅₀ value between 1 μM and 9 μM.

TABLE 2
mHTT protein lowering

| | mHTT | | mHTT |
|---|---|---|---|
| Cpd ID | IC₅₀ | Cpd ID | IC₅₀ |
| 1 | A | 80 | B |
| 2 | A | 81 | C |
| 3 | A | 82 | A |
| 4 | B | 83 | B |
| 5 | B | 84 | A |
| 6 | B | 85 | B |
| 7 | C | 86 | C |
| 8 | C | 87 | B |
| 9 | C | 88 | B |
| 10 | C | 89 | B |
| 11 | B | 90 | C |
| 12 | B | 91 | A |
| 13 | B | 92 | A |
| 14 | B | 93 | B |
| 15 | B | 94 | B |
| 16 | B | 95 | B |
| 17 | B | 96 | C |
| 18 | C | 97 | B |
| 19 | C | 98 | C |
| 20 | C | 99 | A |
| 21 | C | 100 | A |
| 22 | C | 101 | C |
| 23 | C | 102 | B |
| 24 | C | 103 | B |
| 25 | B | 104 | C |
| 26 | B | 105 | C |
| 27 | B | 106 | A |
| 28 | C | 107 | B |
| 29 | B | 108 | A |
| 30 | A | 109 | A |
| 31 | A | 110 | A |
| 32 | B | 111 | A |
| 33 | C | 112 | A |
| 34 | C | 113 | A |
| 35 | C | 114 | A |
| 36 | C | 115 | B |
| 37 | C | 116 | C |
| 38 | C | 117 | B |
| 39 | C | 118 | A |
| 40 | C | 119 | A |
| 41 | C | 120 | B |
| 42 | C | 121 | B |
| 43 | C | 122 | C |
| 44 | C | 123 | B |
| 45 | A | 124 | C |
| 46 | A | 125 | B |
| 47 | A | 126 | A |
| 48 | C | 127 | C |
| 49 | C | 128 | A |
| 50 | C | 129 | C |
| 51 | C | 130 | C |
| 52 | C | 131 | B |
| 53 | C | 132 | C |
| 54 | C | 133 | C |
| 55 | C | 134 | C |
| 56 | C | 135 | B |
| 57 | C | 136 | B |
| 58 | C | 137 | B |
| 59 | C | 138 | B |
| 60 | A | 139 | A |
| 61 | A | 140 | A |
| 62 | A | 141 | C |
| 63 | C | 142 | C |
| 64 | C | 143 | C |
| 65 | C | 144 | C |
| 66 | C | 145 | C |
| 67 | C | 146 | C |
| 68 | C | 147 | C |
| 69 | A | 148 | B |
| 70 | C | 149 | C |
| 71 | B | 150 | A |
| 72 | C | 151 | C |
| 73 | C | 152 | A |
| 74 | B | 153 | A |
| 75 | C | 154 | A |
| 76 | C | 155 | C |
| 77 | C | 156 | C |
| 78 | A | 157 | B |
| 79 | B | 246 | A |
| 158 | A | 247 | B |
| 159 | B | 248 | A |
| 160 | A | 249 | A |
| 161 | A | 250 | B |
| 162 | B | 251 | B |
| 163 | B | 252 | B |
| 164 | B | 253 | B |
| 165 | A | 254 | A |
| 166 | A | 255 | B |
| 167 | A | 256 | B |
| 168 | A | 257 | A |
| 169 | B | 258 | A |
| 170 | A | 259 | B |
| 171 | A | 260 | B |
| 172 | B | 261 | A |
| 173 | B | 262 | A |
| 174 | B | 263 | A |
| 175 | B | 264 | A |
| 176 | A | 265 | A |
| 177 | A | 266 | A |
| 178 | A | 267 | B |
| 179 | B | 268 | A |
| 180 | A | 269 | A |
| 181 | A | 270 | A |
| 182 | A | 271 | A |
| 183 | B | 272 | B |
| 184 | B | 273 | A |
| 185 | A | 274 | A |
| 186 | B | 275 | A |
| 187 | A | 276 | B |
| 188 | B | 277 | A |
| 189 | A | 278 | A |
| 190 | B | 279 | A |
| 191 | A | 280 | A |
| 192 | A | 281 | A |
| 193 | A | 282 | A |
| 194 | A | 283 | B |
| 195 | A | 284 | B |
| 196 | B | 285 | B |
| 197 | A | 286 | A |
| 198 | B | 287 | A |
| 199 | B | 288 | A |
| 200 | A | 289 | A |
| 201 | B | 290 | A |
| 202 | A | 291 | B |
| 203 | A | 292 | A |
| 204 | B | 293 | A |
| 205 | B | 294 | A |
| 206 | A | 295 | A |
| 207 | A | 296 | A |
| 208 | B | 315 | B |
| 209 | A | 316 | B |
| 210 | B | 317 | B |
| 211 | A | 318 | C |
| 212 | A | 319 | C |
| 213 | B | 320 | C |
| 215 | A | 229 | A |
| 218 | B | 321 | B |
| 219 | B | 322 | B |
| 220 | A | 231 | A |
| 221 | A | 235 | A |
| 222 | A | 323 | B |
| 223 | B | 324 | C |
| 224 | A | 325 | B |
| 225 | A | 326 | B |
| 226 | B | 327 | A |
| 227 | A | 328 | B |
| 228 | B | 329 | B |
| 230 | A | 330 | B |
| 232 | A | 331 | A |
| 233 | A | 332 | A |
| 234 | A | 334 | B |
| 236 | B | 335 | B |
| 237 | A | 338 | A |
| 238 | A | 339 | A |
| 216 | B | 374 | A |
| 350 | A | 375 | B |
| 351 | B | 376 | B |
| 352 | C | 377 | B |
| 348 | B | 378 | B |
| 353 | B | 379 | B |
| 354 | B | 380 | B |
| 355 | A | 381 | B |
| 356 | B | 382 | B |
| 357 | B | 383 | B |
| 358 | A | 384 | B |
| 359 | A | 385 | B |
| 360 | B | 386 | A |
| 361 | A | 387 | B |
| 362 | A | 388 | B |
| 363 | A | 389 | B |
| 364 | B | 390 | B |
| 365 | A | 391 | A |
| 366 | C | 392 | B |
| 367 | B | 393 | B |
| 368 | B | 394 | B |
| 369 | A | 395 | A |
| 371 | B | 396 | B |
| 372 | A | 397 | B |
| 373 | A | 398 | A |
| 417 | B | 454 | B |
| 418 | B | 455 | B |
| 419 | B | 456 | B |
| 420 | B | 458 | B |
| 421 | B | 459 | A |
| 422 | A | 466 | B |
| 423 | B | 467 | B |
| 424 | B | 445 | B |
| 425 | B | 468 | A |
| 426 | A | 469 | B |
| 427 | A | 470 | A |
| 428 | A | 471 | A |
| 429 | A | 472 | B |
| 430 | A | 473 | B |
| 431 | B | 474 | B |
| 432 | B | 475 | A |
| 433 | B | 217 | B |
| 434 | B | 497 | B |
| 435 | B | 498 | B |
| 436 | B | 499 | B |
| 437 | B | 500 | B |
| 438 | B | 501 | B |
| 439 | B | 502 | A |
| 440 | B | 503 | A |
| 441 | B | 505 | A |
| 442 | B | 506 | A |
| 443 | A | 507 | A |
| 444 | B | 508 | B |
| 446 | B | 509 | B |
| 447 | B | 542 | B |
| 448 | B | 543 | C |
| 449 | B | 539 | A |
| 450 | B | 544 | C |
| 451 | B | 545 | C |
| 452 | B | 546 | C |
| 453 | B | 547 | C |
| 504 | B | 548 | C |
| 476 | B | 549 | B |
| 477 | B | 550 | B |
| 478 | A | 552 | B |
| 479 | A | 554 | B |
| 480 | A | 555 | B |
| 481 | A | 556 | B |
| 482 | A | 557 | A |
| 483 | B | 551 | A |
| 484 | A | 532 | C |
| 485 | A | 533 | C |
| 486 | A | 534 | C |
| 487 | A | 535 | C |
| 488 | B | 536 | B |
| 489 | A | 537 | A |
| 553 | B | 559 | B |
| 490 | B | 561 | A |
| 491 | B | 514 | A |
| 492 | A | 515 | B |
| 493 | B | 520 | A |
| 494 | B | 577 | A |
| 495 | A | | |
| 496 | B | | |
| 538 | A | | |
| 540 | A | | |
| 541 | B | | |

What is claimed is:

1. A compound of Formula (I): 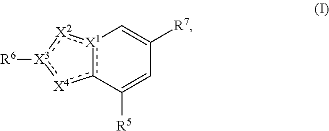
or a pharmaceutically acceptable salt thereof, wherein: [symbol]
is a single bond or double bond, provided the ring containing X₁, X₂, X₃, and X₄ is a bicyclic heteroaryl ring comprising at least one N atom;
X¹ is C or N;
X² is O, N or CR²;
X³ is N or C;
X⁴ is N, O, NR⁴ or CR⁴; provided when X¹ is C, at least two of X², X³ and X⁴ are 0, N or NR⁴;
R² and R⁴, when present, are each independently selected from a group consisting of H, halo, and C₁₋₆alkyl;
R⁵ is H, halo, hydroxyl, C₁₋₆alkyl, C₁₋₆haloalkyl, C₁₋₆alkoxyl, or C₁₋₆haloalkoxyl;
R⁶ is A, —N(R^{6a})-A, —C(═O)A, —N(R^{6a})C(═O)-A, or —C(═O)N(R^{6a})-A and R⁷ is B; additionally R⁶ is B and R⁷ is A when X¹ is N; wherein
R^{6a} is H or C₁₋₃alkyl;
A is —C₁₋₆alkylene-NR⁹R¹⁰, 4 to 10 membered carbocyclyl, —C₁₋₆alkylene-(4 to 10 membered carbocyclyl), Het or —C₁₋₆alkylene-Het; wherein
R⁹ is H or C₁₋₆alkyl;
R¹⁰ is H, C₁₋₆alkyl, C₃₋₆cycloalkyl, —C₁₋₆alkylene-C₃₋₆cycloalkyl, or —C₁₋₆alkylene-Het¹, wherein Het¹ is a 4-6 membered saturated heterocyclyl;
Het is a 4 to 12 membered saturated heterocyclyl optionally substituted with —NR⁹R¹⁰ or —C₁₋₆alkylene-NR⁹R¹⁰ and optionally further substituted with 1 to 4 R¹¹;
said 4 to 10 membered carbocyclyl represented by A is optionally substituted by —NR⁹R¹⁰ or —C₁₋₆alkylene-NR⁹R¹⁰ and is further optionally substituted with 1 to 2 R¹¹; wherein
R¹¹, for each occurrence, is independently selected from halo, —C(═O)R¹², C₁₋₆alkyl, C₁₋₆haloalkyl, C₁₋₆alkoxyC₁₋₆alkyl, C₃₋₆cycloalkyl, —C₁₋₆alkylene-C₃₋₆cycloalkyl, Het², and —C₁₋₆alkylene-Het², wherein Het² is a 4 to 6 membered saturated heterocyclyl or 5 to 10 member heteroaryl, wherein said Het² or C₃₋₆cycloalkyl is optionally substituted by one or more substituents independently selected from halo, C₁₋₆alkoxy and C₁₋₆alkyl; wherein R¹² is H, D, halo, C₁₋₃alkyl, C₁₋₆alkoxyl, or C₃₋₆cycloalkyl;
B is 6 to 10 membered aryl, 4 to 10 membered heterocyclyl, or 5 to 10 member heteroaryl, wherein said 6 to 10 membered aryl, 4 to 10 membered heterocyclyl, and 5 to 10 member heteroaryl represented by B are optionally substituted by one or more R⁸; wherein
R⁸ is halo, —CN, —OH, C₁₋₆alkyl, C₃₋₆cycloalkyl, 5 or 6 membered heteroaryl, C₁₋₆haloalkyl, or C₁₋₆alkoxy, or two R⁸ together with the intervening atoms together form a 4 to 7 membered heterocyclyl optionally substituted with one or more R^{8b}; wherein said 5 or 6 membered heteroaryl represented by R⁸ is optionally substituted by one or more R^{8a}; wherein R^{8a} is C₁₋₃alkyl; and R^{8b} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur.

2. The compound of claim 1, wherein the compound represented by Formula (I): 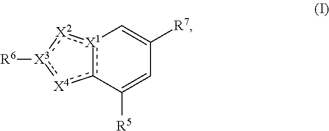
or a pharmaceutically acceptable salt thereof, wherein: [symbol]
is a single bond or double bond, provided the ring containing X₁, X₂, X₃, and X₄ is a bicyclic heteroaryl ring comprising at least one N atom;
X¹ is C or N;
X² is O, N or CR²;
X³ is N or C;
X⁴ is N, NR⁴ or CR⁴; provided when X¹ is C, at least two of X², X³ and X⁴ are O, N or NR⁴;
R² and R⁴, when present, are each independently selected from a group consisting of H, halo, and C₁₋₆alkyl;
R⁵ is halo;
R⁶ is A, —N(R^{6a})C(═O)-A, or —C(═O)N(R^{6a})-A and R⁷ is B; or
R⁶ is B and R⁷ is A when X¹ is N; wherein
R^{6a} is H or C₁₋₃alkyl;
A is —C₁₋₆alkylene-NR⁹R¹⁰, 4 to 10 membered saturated carbocyclyl, Het or —C₁₋₆alkylene-Het; wherein
R⁹ is H or C₁₋₆alkyl;
R¹⁰ is H, C₁₋₆alkyl or —C₁₋₆alkylene-Het¹, wherein Het¹ is a 4-6 membered saturated heterocyclyl;
Het is a 4 to 10 membered saturated heterocyclyl, provided when said 4 to 10-membered saturated heterocyclyl represented by Het does not comprise a ring N atom, it is then substituted with —NR⁹R¹⁰ and optionally further substituted with 1 to 2 R¹¹, and when the 4 to 10-membered saturated heterocyclyl represented by Het comprises one or more ring N atoms, it is optionally substituted with 1 to 3 R¹¹;
said 4 to 10 membered saturated carbocyclyl represented by A is substituted by —NR⁹R¹⁰ and is further optionally substituted with 1 to 2 R¹¹; wherein
R¹¹, for each occurrence, is independently selected from halo, —C(═O)R¹², C₁₋₄alkyl, C₁₋₆haloalkyl, C₁₋₆alkoxyC₁₋₆alkyl, and C₃₋₆cycloalkyl; wherein said C₃₋₆cycloalkyl represented by R¹¹ is optionally substituted by one or more substituents independently selected from halo and C₁₋₆alkyl; wherein R¹² is H, C₁₋₃alkyl, or C₃₋₆cycloalkyl;
B is 6 to 10 membered aryl, 4 to 10 membered heterocyclyl, or 5 to 10 member heteroaryl, wherein said 6 to 10 membered aryl, 4 to 10 membered heterocyclyl, and 5 to 10 member heteroaryl represented by B are optionally substituted by one or more R⁸; wherein
R⁸ is halo, —CN, —OH, C₁₋₆alkyl, C₁₋₆haloalkyl, or C₁₋₆alkoxy, or two R⁸ together with the intervening atoms together form a 5 to 7 membered heterocyclyl optionally substituted with one or more R^{8b}; wherein said 5 or 6 membered heteroaryl represented by R⁸ is optionally substituted by one or more R^{8a}; wherein R^{8a} is C₁₋₃alkyl; and R^{8b} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur.

3. The compound of claim 1 or 2, or a pharmaceutically acceptable salt thereof, wherein the compound is represented by Formula (II), (III), (IV), (V), (VI), (VII), (VIII), or (IX):

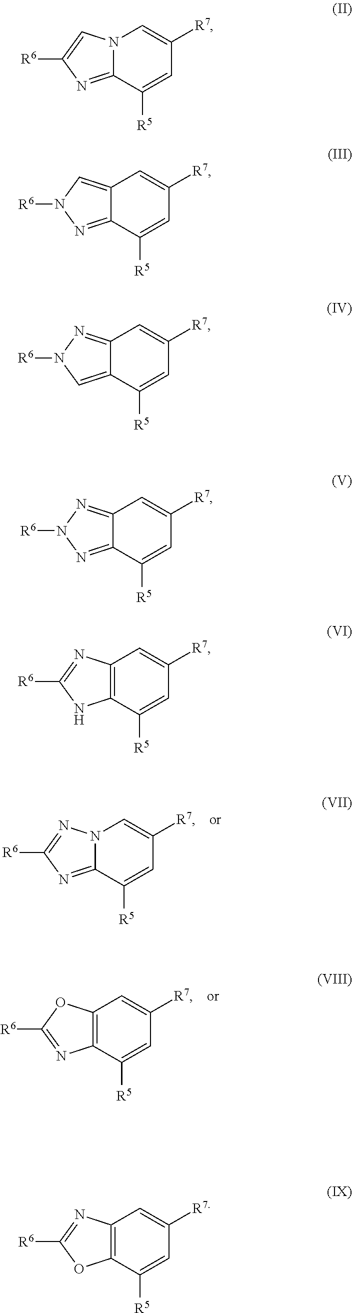

4. The compound of claim 1 or 2, or a pharmaceutically acceptable salt thereof, wherein the compound is represented by Formula (II), (III), (IV), (V), (VI), (VII), or (VIII):

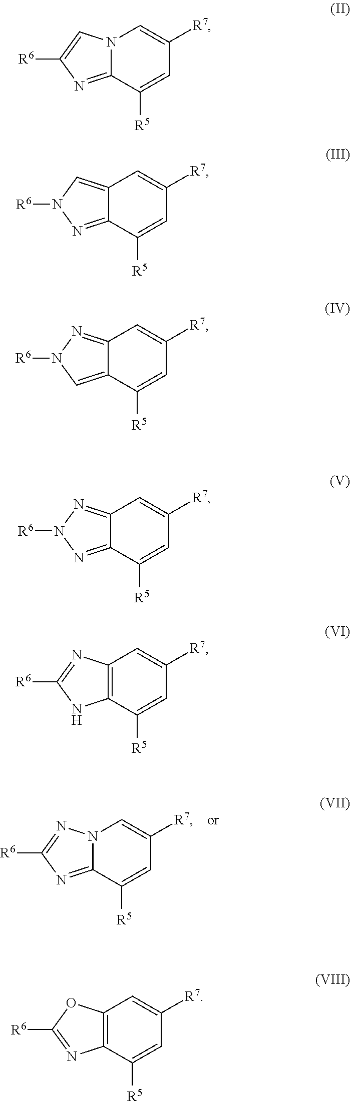

5. The compound of any one of claims 1-4, or a pharmaceutically acceptable salt thereof, wherein the compound is represented by Formula (II):

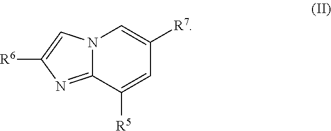

6. The compound of any one of claims 1-5 or a pharmaceutically acceptable salt thereof, wherein R⁵ is H, halo, C₁₋₃alkyl, C₁₋₃haloalkoxyl or C₁₋₃alkoxyl.

7. The compound of any one of claims 1-6 or a pharmaceutically acceptable salt thereof, wherein R⁵ is F or Cl.

8. The compound of any one of claims 1-6 or a pharmaceutically acceptable salt thereof, wherein R⁵ is H, F, Cl, —CH₃, —OCHF₂, —OCH₃ or —OCF₃.

9. The compound of any one of claims 1-8 or a pharmaceutically acceptable salt thereof, wherein R⁵ is F.

10. The compound of any one of claims 1-9 or a pharmaceutically acceptable salt thereof, wherein R⁶ is A, —N(R^{6a})-A, —N(R^{6a})C(═O)-A, or —C(═O)N(R^{6a})-A; R⁷ is B and R^{6a} is H or —CH₃.

11. The compound of any one of claims 1-10 or a pharmaceutically acceptable salt thereof, wherein R⁶ is A, —NHC(═O)-A, or —C(═O)NH-A and R⁷ is B.

12. The compound of any one of claims 1-9 or a pharmaceutically acceptable salt thereof, wherein R⁶ is B and R⁷ is A when X¹ is N.

13. The compound of claim 12 or a pharmaceutically acceptable salt thereof; wherein
X¹ is N;
X² is CR²;
X³ is C;
X is N;
R⁶ is B; and
R⁷ is A.

14. The compound of any one of claims 1-11 or a pharmaceutically acceptable salt thereof, wherein R⁶ is A when R⁷ is B.

15. The compound of any one of claims 1-11, or a pharmaceutically acceptable salt thereof, wherein R⁶ is —N(R^{6a})-A, —N(R^{6a})C(═O)-A or —C(═O)N(R^{6a})-A and R⁷ is B when X¹ is N; and wherein R^{6a} is H or —CH₃.

16. The compound of any one of claims 1-11, or a pharmaceutically acceptable salt thereof, wherein R⁶ is —NHC(═O)-A or —C(═O)NH-A and R⁷ is B when X¹ is N.

17. The compound of claim 15 or a pharmaceutically acceptable salt thereof, wherein
X¹ is N;
X² is CR²;
X³ is C;
X is N;
R⁶ is —N(R^{6a})-A, —N(R^{6a})C(═O)-A or —C(═O)N(R^{6a})-A;
R^{6a} is H or —CH₃; and
R⁷ is B.

18. The compound of claim 16 or a pharmaceutically acceptable salt thereof, wherein
X¹ is N;
X² is CR²;
X³ is C;
X is N;
R⁶ is —NHC(═O)-A or —C(═O)NH-A; and
R⁷ is B.

19. The compound of any one of claims 1-18 or a pharmaceutically acceptable salt thereof, wherein
A is —C₁₋₆alkylene-C₃₋₆cycloalkyl, —C₁₋₆alkylene-NR⁹R¹⁰ or —C₁₋₆alkylene-Het; wherein
said Het in —C₁₋₆alkylene-Het represented by A is a 4 to 6 membered monocyclic saturated heterocyclyl comprising a ring N atom; and
R⁹ and R¹⁰ are each independently H or C₁₋₄alkyl.

20. The compound of claim 19 or a pharmaceutically acceptable salt thereof, wherein Het in —C₁₋₆alkylene-Het represented by A is azetidinyl, pyrrolidinyl, piperidinyl, morpholinyl or piperazinyl.

21. The compound of claim 19 or a pharmaceutically acceptable salt thereof, wherein A is selected from a group consisting of

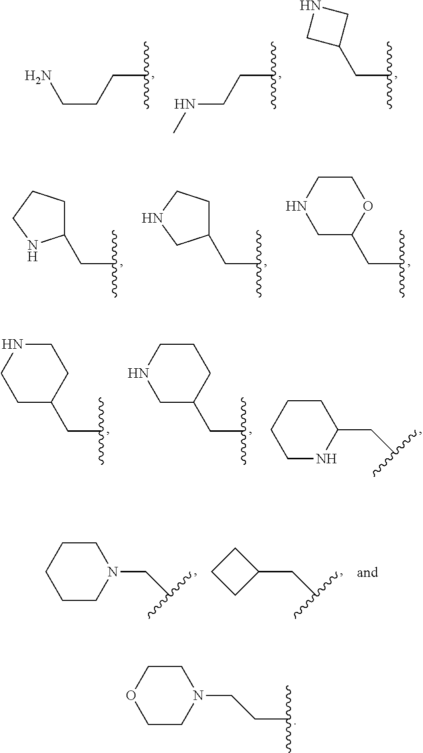

22. The compound of claim 19, or a pharmaceutically acceptable salt thereof, wherein A is selected from a group consisting of

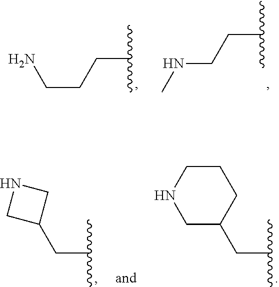

23. The compound of any one of claims 1-18 or a pharmaceutically acceptable salt thereof, wherein:
A is 5 to 6 membered monocyclic carbocyclyl, 5 to 8 membered bicyclic saturated bridged carbocyclyl, or Het;
Het represented by A is a 4 to 7 membered monocyclic saturated heterocyclyl, 6 to 8 membered bicyclic saturated bridged heterocyclyl, or 7 to 12 membered bicyclic saturated spiral or fused heterocyclyl; provided when the Het represented by A does not comprise a ring N atom, it is then substituted with —NR⁹R¹⁰ or —C₁₋₆alkylene-NR⁹R¹⁰ and optionally further substituted with 1 to 2 R¹¹, and when the Het represented by A comprises one or more ring N atoms, it is optionally substituted with 1 to 2 R¹¹; and
said 5 to 8 membered bicyclic saturated bridged carbocyclyl represented by A is substituted by —NR⁹R¹⁰, 4 to 6 membered monocyclic saturated heterocyclyl, or —C₁₋₆ alkylene-NR⁹R¹⁰ and is optionally further substituted with 1 to 2 R¹¹;
R⁹ and R¹⁰ are each independently H or C₁₋₄alkyl.

24. The compound of any one of claims 1-18 or a pharmaceutically acceptable salt thereof, wherein:
A is 5 to 6 membered monocyclic saturated carbocyclyl, 5 to 8 membered bicyclic saturated bridged carbocyclyl, or Het;
Het represented by A is a 4 to 6 membered monocyclic saturated heterocyclyl, 6 to 8 membered bicyclic saturated bridged heterocyclyl, or 7 to 10 membered bicyclic saturated spiral heterocyclyl; provided when the Het represented by A does not comprise a ring N atom, it is then substituted with —NR⁹R¹⁰ and optionally further substituted with 1 to 2 R¹¹, and when the Het represented by A comprises one or more ring N atoms, it is optionally substituted with 1 to 2 R¹¹; and
said 5 to 8 membered bicyclic saturated bridged carbocyclyl represented by A is substituted by —NR⁹R¹⁰ and is optionally further substituted with 1 to 2 R¹¹;
R⁹ and R¹⁰ are each independently H or C₁₋₄alkyl.

25. The compound of claim 24 or a pharmaceutically acceptable salt thereof, wherein A is azetidinyl, pyrrolidinyl, piperidinyl, morpholinyl, piperazinyl, 2-azabicyclo[2.1.1]hexyl, 3-azabicyclo[3.1.1]heptanyl, 2-azabicyclo[3.1.1]heptanyl, 2-azabicyclo[2.2.1]heptanyl, 1-azaspiro[3.3]heptanyl, 2-azaspiro[4.5]decanyl, 4-azaspiro[2.5]octanyl, 8-azaspiro[4.5]decanyl, 8-azabicyclo[3.2.1]octanyl, 3-azabicyclo[3.2.1]octanyl, 9-diazaspiro[5.5]undecanyl, 2-azabicyclo[4.1.0]heptanyl, 3-azabicyclo[4.1.0]heptanyl, 5-azaspiro[2.4]heptanyl, 5-azaspiro[2.3]hexanyl, 4-azaspiro[2.4]heptanyl, 6-azaspiro[3.4]octanyl, 2-azaspiro[4.4]nonanyl, 2-azaspiro[3.5]nonanyl, 2-azaspiro[3.4]octanyl, 1-oxa-9-azaspiro[5.5]undecanyl, 3-azabicyclo[3.1.0]hexanyl, diazaspiro[4.5]decane, 7-diazaspiro[3.5]nonanyl, diazaspiro[4.5]decanyl, 7-diazaspiro[4.4]nonanyl, 1-azabicyclo[3.2.1]octanyl, diazaspiro[5.5]undecanyl, azepanyl, 7-azaspiro[3.5]nonanyl, 5-oxa-2-azaspiro[3.4]octanyl, diazabicyclo[3.2.0]heptanyl, 3-azabicyclo[3.2.0]heptanyl, octahydro-cyclopenta[c]pyrrolyl, hexahydro-1H-pyrrolo[3,4-c]pyrrolyl, octahydro-indolizinyl, 8-diazabicyclo[4.2.0]octanyl, octahydro-isoindolyl, or 1,8-diazaspiro[4.5]decane, each of which is optionally substituted with one or two R¹¹.

26. The compound of claim 24 or a pharmaceutically acceptable salt thereof, wherein A is azetidinyl, pyrrolidinyl, piperidinyl, morpholinyl, piperazinyl, 2-azabicyclo[2.1.1]hexyl, 3-azabicyclo[3.1.1]heptanyl, 2-azabicyclo[3.1.1]heptanyl, 2-azabicyclo[2.2.1]heptanyl, 1-azaspiro[3.3]heptanyl, 2-azaspiro[4.5]decanyl, 4-azaspiro[2.5]octanyl, 8-azaspiro[4.5]decanyl, 8-azabicyclo[3.2.1]octanyl, or 7-azaspiro[3.5]nonanyl, each of which is optionally substituted with one or two R¹¹.

27. The compound of claim 23 or 25 or a pharmaceutically acceptable salt thereof, wherein A is selected from a group consisting of: 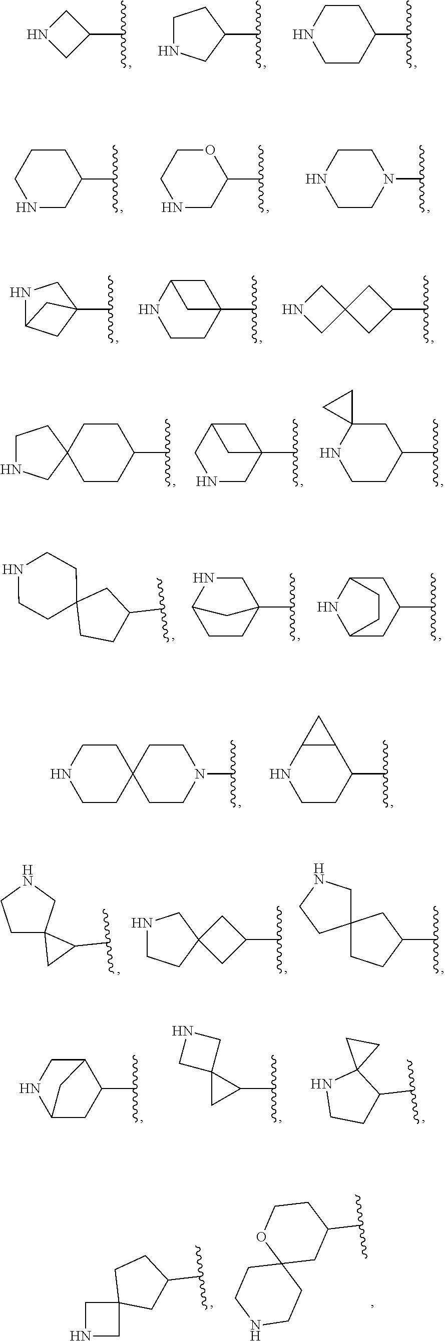 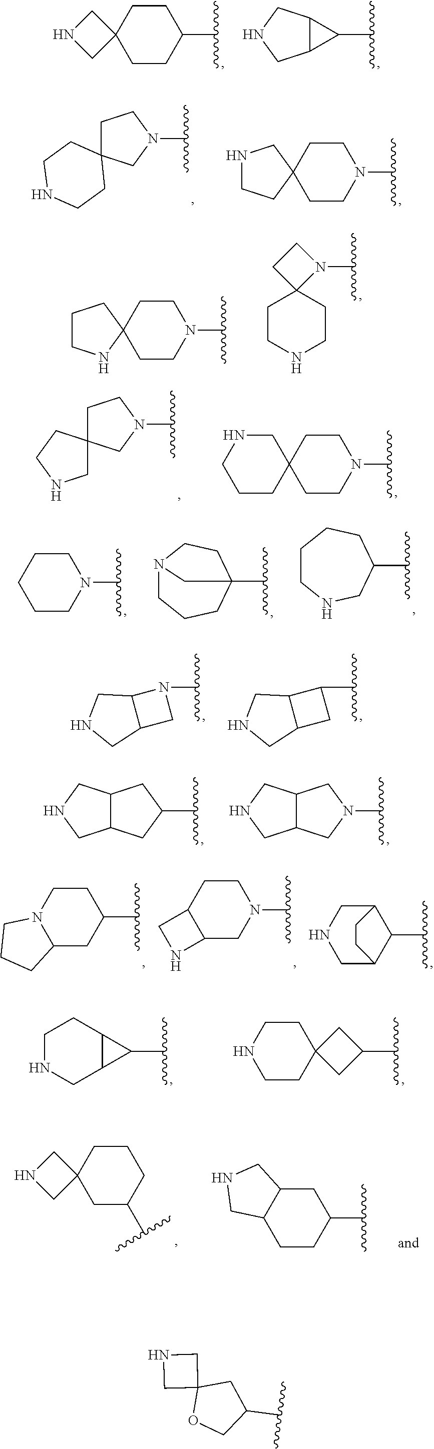
and p each of which is optionally substituted with one or two R¹¹.

28. The compound of claim 24 or 26 or a pharmaceutically acceptable salt thereof, wherein A is selected from a group consisting of: 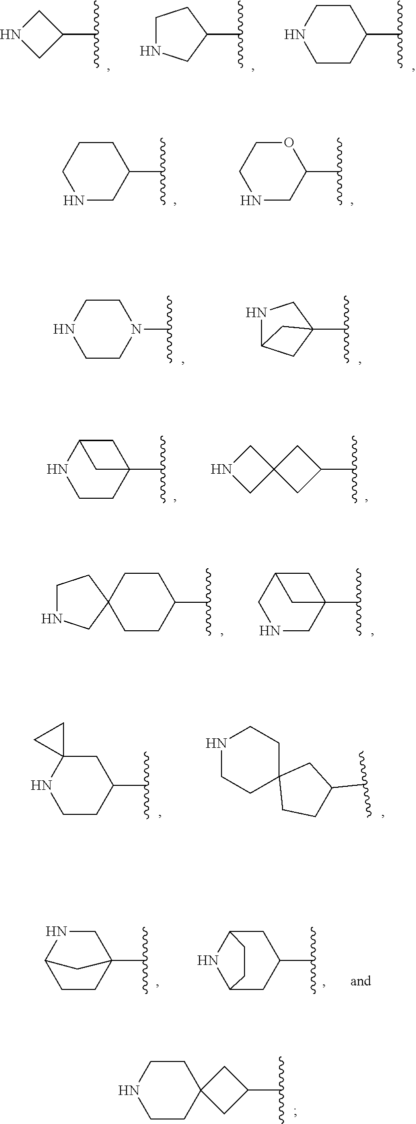
each of which is optionally substituted with one or two R¹¹.

29. The compound of claim 23 or a pharmaceutically acceptable salt thereof, wherein A is cyclobutyl, cyclopentyl, cyclopenetenyl, cyclohexyl, tetrahydro-2H-pyranyl, 3-oxetanyl, bicycle[1.1.1]pentyl, bicycle[2.1.1]hexyl, bicyclo[3.2.0]heptanyl, —CH₂-cyclobutyl, bicyclo[2.2.2]octanyl, spiro[5.3]nonanyl, or 2-oxobicyclo[2.1.1]hexyl, each of which is substituted with —NR⁹R¹⁰ and optionally further substituted with 1 to 2 R¹¹.

30. The compound of claim 24 or a pharmaceutically acceptable salt thereof, wherein A is cyclopentyl, bicycle[1.1.1]pentyl, bicycle[2.1.1]hexyl, bicyclo[2.2.2]octanyl, or 2-oxobicyclo[2.1.1]hexyl, each of which is substituted with —NR⁹R¹⁰ and optionally further substituted with 1 to 2 R¹¹.

31. The compound of claim 23 or 29 or a pharmaceutically acceptable salt thereof, wherein A is selected from a group consisting of 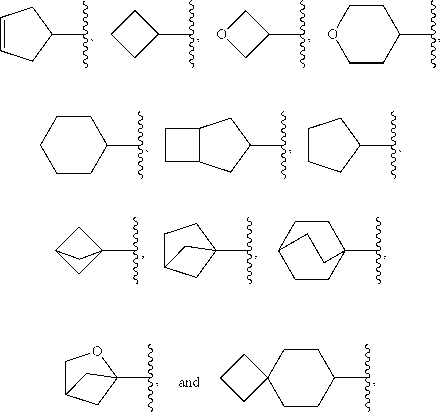
each of which is substituted with —NR⁹R¹⁰ and optionally further substituted with 1 to 2 R¹¹.

32. The compound of claim 24 or 30 or a pharmaceutically acceptable salt thereof, wherein A is selected from a group consisting of 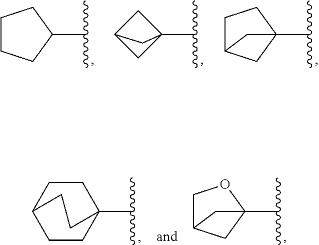
each of which is substituted with —NR⁹R¹⁰ and optionally further substituted with 1 to 2 R¹¹.

33. The compound of any one of claims 1-32 or a pharmaceutically acceptable salt thereof, wherein R¹¹, for each occurrence, is independently selected from halo, —C(═O)R¹², C₁₋₆alkyl, C₁₋₄alkoxyC₁₋₄alkyl, C₃₋₆cycloalkyl, —C₁₋₆alkylene-C₃₋₆cycloalkyl, Het², —C₁₋₆alkylene-Het², wherein Het² is a 4-6 membered saturated heterocyclyl or 5 to 6 membered heteroaryl; wherein said C₃₋₆cycloalkyl or Het² represented by R¹¹ is optionally substituted by one to four substituents independently selected from halo, C₁₋₄alkoxy and C₁₋₄alkyl; and R¹² is H, D, halo, C₁₋₄alkoxyl, C₁₋₂alkyl, C₃₋₄cycloalkyl.

34. The compound of any one of claims 1-32 or a pharmaceutically acceptable salt thereof, wherein R¹¹, for each occurrence, is independently selected from halo, —C(═O)R¹², C₁₋₄alkyl, C₁₋₄alkoxyC₁₋₄alkyl, C₃₋₆cycloalkyl; wherein said C₃₋₆cycloalkyl represented by R¹¹ is optionally substituted by one to three substituents independently selected from F, Cl, and C₁₋₄alkyl; and R¹² is H, C₁₋₂alkyl, C₃₋₄cycloalkyl.

35. The compound of any one of claims 1-32 or a pharmaceutically acceptable salt thereof, wherein R¹¹, for each occurrence, is independently selected from F, —C(═O)CH₃, —C(═O)CH₂CH₃, —C(═O)cyclopropyl, —CH₃, —CH₂CH₃, —CH(CH₃)₂, —C(CH₃)₃, —CH₂C(CH₃)₃, —CH₂CH₂OCH₃, —CH₂ CH₂CH₂OCH₃, —CH₂CH₂CH₂OCH₃, —CH₂CHF₂, —CH₂CH₂F, —CH₂-cyclopropyl, —CH₂-cyclobutyl, cyclopropyl, cyclobutyl and cyclopentyl, 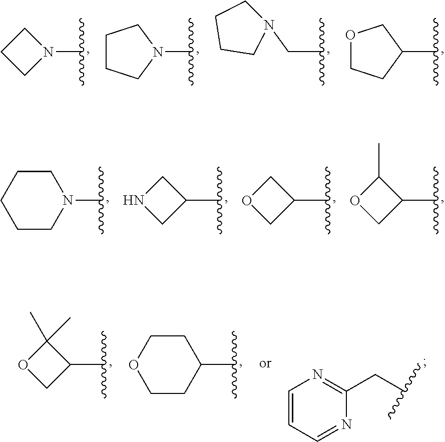
wherein said cyclopropyl, cyclobutyl, or cyclopentyl represented by R¹¹ is optionally substituted by one to two substituents independently selected from D, F, C₁₋₃alkoxy and C₁₋₃alkyl.

36. The compound of any one of claims 1-32 or a pharmaceutically acceptable salt thereof, wherein R¹¹, for each occurrence, is independently selected from F, —C(═O)CH₃, —C(═O)CH₂CH₃, —C(═O)cyclopropyl, —CH₃, —CH₂CH₃, —CH(CH₃)₂, —CH₂CH₂OCH₃, —CH₂CH₂CH₂OCH₃, cyclopropyl, and cyclobutyl; wherein said cyclopropyl represented by R¹¹ is optionally substituted by one to two substituents independently selected from F and C₁₋₃alkyl.

37. The compound of any one of claims 1-36 or a pharmaceutically acceptable salt thereof, wherein R⁹ is H or C₁₋₃alkyl and R¹⁰ is H, C₃₋₆cycloalkyl or C₁₋₃alkyl.

38. The compound of any one of claims 1-36 or a pharmaceutically acceptable salt thereof, wherein R⁹ and R¹⁰ are each independently H or C₁₋₃alkyl.

39. The compound of any one of claims 1-36 or a pharmaceutically acceptable salt thereof, wherein R⁹ is H or —CH₃ and R¹⁰ is H, cyclopropyl or —CH₃.

40. The compound of any one of claims 1-36 or a pharmaceutically acceptable salt thereof, wherein R⁹ and R¹⁰ are each independently H or —CH₃.

41. The compound of any one of claims 1-40 or a pharmaceutically acceptable salt thereof, wherein B is phenyl, naphthalenyl, or 8 to 10 membered bicyclic heteroaryl; wherein said phenyl, naphthalenyl, and 8 to 10 membered bicyclic heteroaryl represented by B are optionally substituted by one to three R⁸.

42. The compound of any one of claims 1-40 or a pharmaceutically acceptable salt thereof, wherein B is 9 or 10 membered bicyclic heteroaryl optionally substituted by one to three R⁸.

43. The compound of any one of claims 1-40 or a pharmaceutically acceptable salt thereof, wherein B is 9 membered bicyclic heteroaryl optionally substituted by one to three R⁸.

44. The compound of any one of claims 1-40 or a pharmaceutically acceptable salt thereof, wherein B is selected from a group consisting of phenyl, indazolyl, imidazopyridinyl, imidazopyridazinyl, benzotriazolyl, imidazopyrazinyl, benzooxazolyl, triazolopyridinyl, benzisothiazolyl, pyrazolopyridinyl, pyrazolopyrazinyl, pyrazolopyrimidinyl, thienopyridinyl, thienopyrimidinyl, benzothiazolyl, pyrrolopyridinyl, pyrrolopyrazinyl, benzofuranyl, benzothiophenyl, isoquinolinyl, pyrrolotriazinyl, thienopyridinyl, triazolopyridazinyl, benzooxadiazolyl, indolyl, indolin-2-onyl, furopyridine, benzoimidazolyl, benzothiadiazole, phthalazinyl and phthalazin-1-onyl, each of which is optionally substituted by one to three R⁸; or
B is 2H-pyrido[3,2-b][1,4]oxazin-3(4H)-onyl, each of which is optionally substituted with C₁₋₃alkyl.

45. The compound of any one of claims 1-40, or a pharmaceutically acceptable salt thereof, wherein B is selected from a group consisting of phenyl, indazolyl, imidazopyridinyl, imidazopyridazinyl, benzothiazolyl, imidazopyrazinyl, benzooxazolyl, triazolopyridinyl, benzisothiazolyl, pyrazolopyridinyl, thienopyridinyl, benzothiazolyl, pyrrolopyridinyl, pyrrolopyrazinyl, benzofuranyl, benzothiophenyl, thienopyridinyl, triazolopyridazinyl, benzooxadiazolyl, indolyl, indolin-2-onyl, furopyridine, benzoimidazolyl, benzothiadiazole, phthalazinyl and phthalazin-1-onyl, each of which is optionally substituted by one to three R⁸; or
B is 2H-pyrido[3,2-b][1,4]oxazin-3(4H)-onyl, each of which is optionally substituted with C₁₋₃alkyl.

46. The compound of claim 44 or a pharmaceutically acceptable salt thereof, wherein B is selected from: 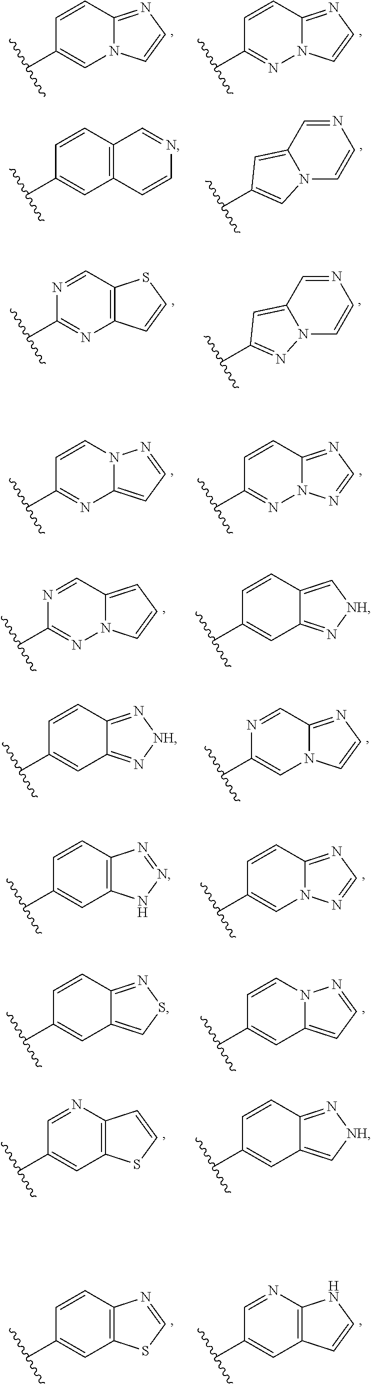 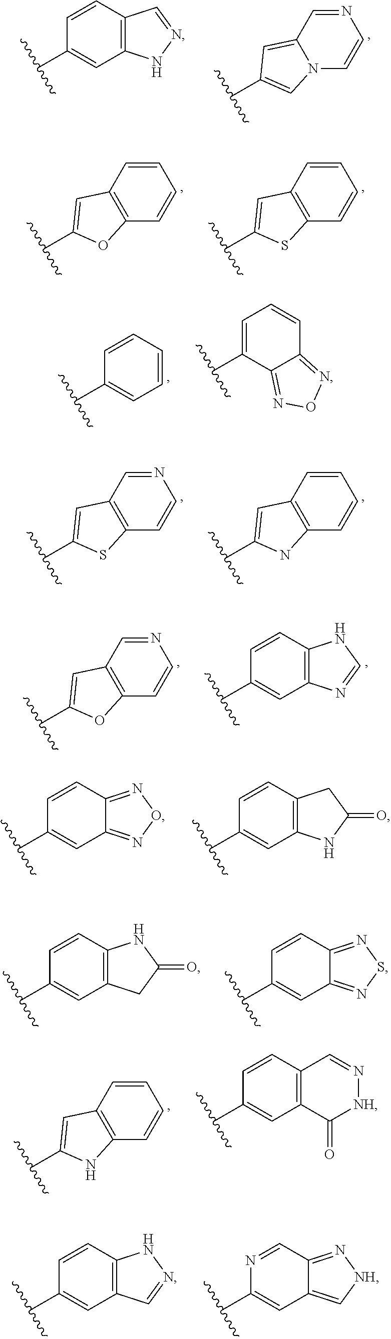 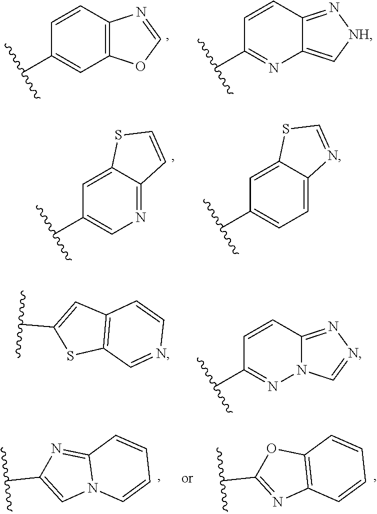
each of which is optionally substituted by one to three R⁸; or
B is

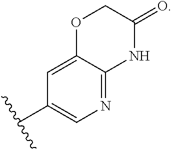 [SMILES: *c1cnc2c(c1)OCC(=O)N2]

47. The compound of claim 45 or a pharmaceutically acceptable salt thereof, wherein B is selected from: 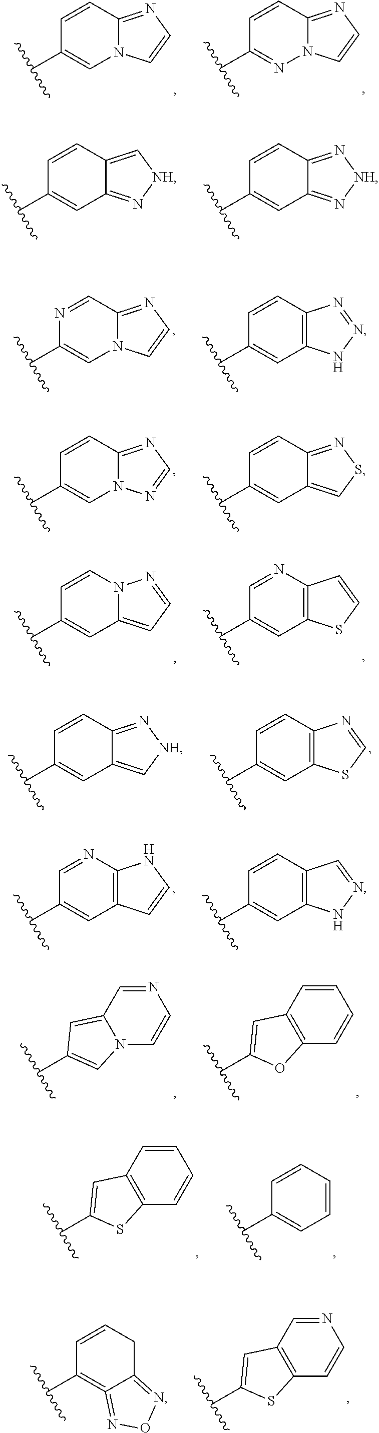 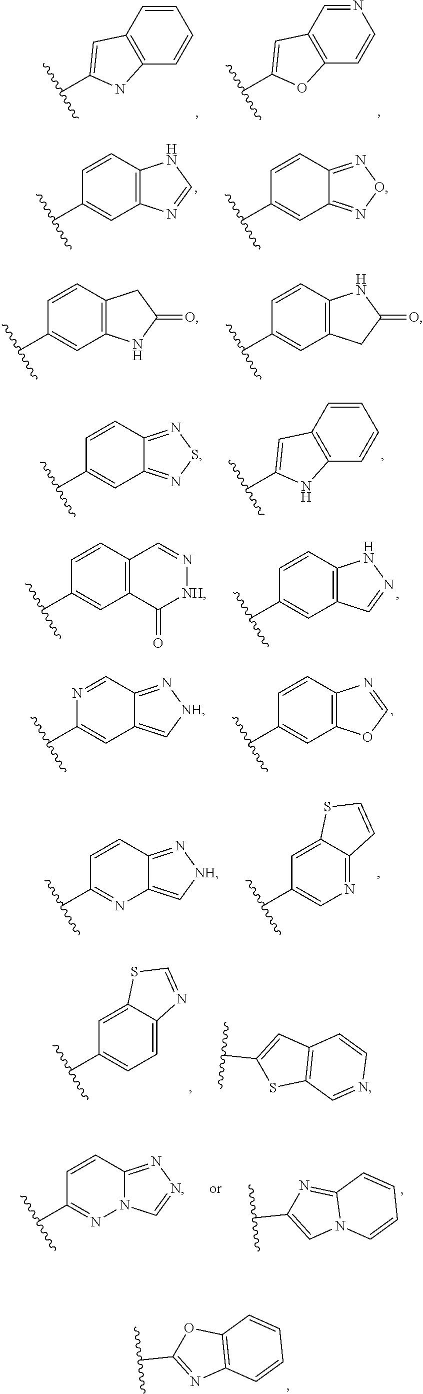
each of which is optionally substituted by one to three R⁸; or
B is

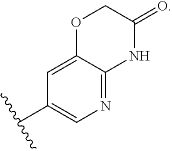 [SMILES: *c1cnc2c(c1)OCC(=O)N2]

48. The compound of any one of claims 1-47 or a pharmaceutically acceptable salt thereof, wherein R⁸ for each occurrence is halo, —CN, —OH, C₁₋₃alkyl, C₃₋₆cycloalkyl, C₁₋₂haloalkyl, or C₁₋₂alkoxy.

49. The compound of any one of claims 1-47 or a pharmaceutically acceptable salt thereof, wherein R⁸ for each occurrence is halo, —CN, —OH, C₁₋₃alkyl, C₁₋₂haloalkyl, or C₁₋₂alkoxy.

50. The compound of claim 48 or a pharmaceutically acceptable salt thereof, wherein R⁸ for each occurrence is independently selected from —F, —Cl, —Br, —CN, —CH₃, —CH₂CH₃, —CH(CH₃)₂, —CHF₂, —CF₃, —OH, —OCH₃, —OCH₂CH₃, and. cyclopropyl

51. The compound of claim 50 or a pharmaceutically acceptable salt thereof, wherein R⁸ for each occurrence is independently selected from —F, —Cl, —CN, —CH₃, —CH₂CH₃, —CH(CH₃)₂, —CHF₂, —OH, —OCH₃, and —OCH₂CH₃.

52. The compound of any one of claims 1-51 or a pharmaceutically acceptable salt thereof, wherein R^{8a} for each occurrence is —CH₃ or —CH₂CH₃; and R⁸ for each occurrence is —CH₃ or oxo.

53. The compound of claim 1 or a pharmaceutically acceptable salt thereof, wherein the compound is selected from Table 1, or a pharmaceutically acceptable salt thereof.

54. The compound of claim 1, wherein the compound is represented by the following formula: 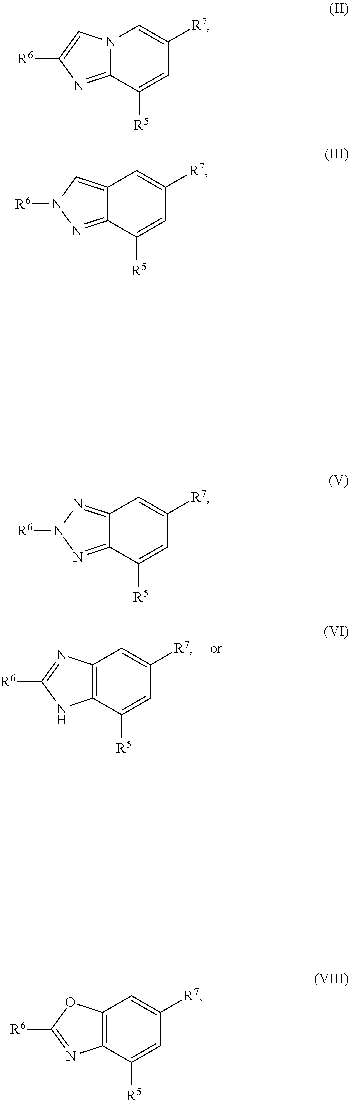
or a pharmaceutically acceptable salt thereof, wherein:
R⁵ is halo;
R⁶ is A, —NH—C(═O)-A, —C(═O)NH-A or —NH-A;
R⁷ is B;
A is 4 to 6 membered monocyclic saturated heterocyclyl, 6 to 10 membered bicyclic saturated fused or spiral heterocyclyl, or C₃₋₆cycloalkyl, wherein the 4 to 6 membered monocyclic saturated heterocyclyl and 6 to 10 membered bicyclic saturated fused or spiral heterocyclyl are each optionally substituted with 1 or 2 R¹¹, and the C₃₋₆cycloalkyl is substituted with —NR⁹R¹⁰ and is further optionally substituted with R¹¹;
R⁹ and R¹⁰ are each independently H or C₁₋₃alkyl;
Each R¹¹ is independently C₁₋₃alkyl or C₃₋₆cycloalkyl,
B is 9-membered bicyclic heteroaryl optionally substituted with 1 to 3 R⁸, wherein the 9-membered bicyclic heteroaryl has 2 to 4 N ring atoms; and
Each R⁸ is independently C₁₋₃alkyl, C₁₋₃haloalkyl or C₁₋₃alkoxy.

55. The compound of claim 54, or a pharmaceutically acceptable salt thereof, wherein:
R⁵ is F;
A is cyclobutyl or Het, wherein Het is azetidinyl, piperidinyl, 3-azabicyclo[3.1.0]hexanyl, 2,8-diazaspiro[4.5]decanyl, or 2,7-azaspiro[3.5]nonanyl, each of which is optionally substituted with C₁₋₃alkyl or C₃₋₆cycloalkyl, and wherein cyclobutyl represented by A is optionally substituted with —NR⁹R¹⁰;
R⁹ and R¹⁰ are each H or —CH₃; and
B is represented by the following formula: 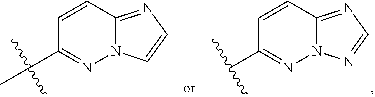 [SMILES: *c1ccc2nccn2n1 ; *c1ccc2ncnn2n1]
each of which is optionally substitute with 1 or 2 R⁸; and
Each R⁸ is independently C₁₋₃alkyl, C₁₋₃haloalkyl or C₁₋₃alkoxy.

56. The claim of claim 55, or a pharmaceutically acceptable salt thereof, wherein A is represented by the following formula: 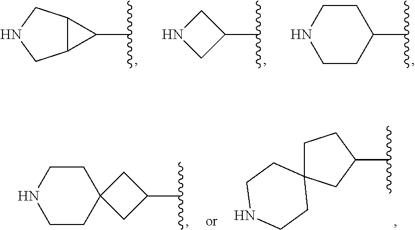 [SMILES: *C1C2CNCC12 ; *C1CNC1 ; *C1CCNCC1 ; *C1CC2(CCNCC2)C1 ; *C1CCC2(CCNCC2)C1]
each of which is optionally substituted with C₁₋₃alkyl.

57. The compound of claim 56, or a pharmaceutically acceptable salt thereof, wherein A is represented by the following formula:

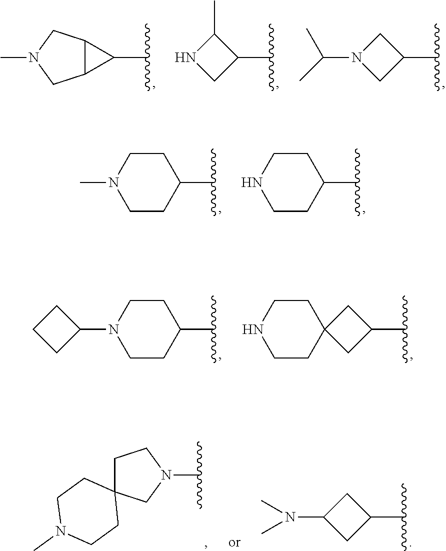

58. The compound of any one of claims 54-57, or a pharmaceutically acceptable salt thereof, wherein B is represented by the following formula:

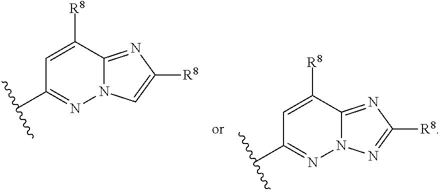

59. The compound of any one of claims 54-58, or a pharmaceutically acceptable salt thereof, each R⁸ is —CH₃, —CHF₂, CF₃, or OCH₃.

60. A pharmaceutical composition comprising a compound of any one of claims 1-59 or a pharmaceutically acceptable salt thereof and a pharmaceutically acceptable carrier.

61. A method of treating Huntington disease (HD) in a subject in need thereof comprising administering to the subject an effective amount of a compound of any one of claims 1-59 or a pharmaceutically acceptable salt thereof or a pharmaceutically composition of claim 60.
